(19) United States
(12) Patent Application Publication
(10) Pub. No.: US 2006/0229236 A1
(43) Pub. Date: Oct. 12, 2006

(54) Cyclic tetrapeptide compound and use thereof

(75) Inventors: Shigeki Satoh, Osaka (JP); Yasuharu Urano, Osaka (JP); Kazuhiko Osoda, Osaka (JP); Mitsuru Hosaka, Osaka (JP); Kozo Sawada, Osaka (JP); Takayuki Inoue, Osaka (JP); Hiroaki Mori, Osaka (JP); Shoji Takagaki, Osaka (JP); Takao Fujimura, Osaka (JP); Hideaki Matsuoka, Osaka (JP); Katsuhiko Yoshizawa, Osaka (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: FUJISAWA PHARMACEUTICAL Co. LTD., OSAKI-SHI (JP)

(21) Appl. No.: 10/500,113

(22) PCT Filed: Dec. 27, 2002
(86) PCT No.: PCT/JP02/13754

§ 371 Date: Feb. 8, 2005

(30) Foreign Application Priority Data
Dec. 28, 2001 (AU) PR 9779
Oct. 10, 2002 (AU) 2002952117

(51) Int. Cl. A61K 38/12; C07K 5/12

(52) U.S. Cl. 514/9; 530/317

(57) ABSTRACT

A cyclic tetrapeptide compound of the formula (I): wherein R₁ is hydrogen; R₂ is lower alkyl, aryl, optionally substituted ar(lower)alkyl, heterocyclic(lower)alkyl, cyclo(lower)alkyl(lower)alkyl, lower alkylcarbamoyl(lower)alkyl or arylcarbamoyl(lower)alkyl; R₃ and R₄ are each independently hydrogen, lower alkyl, optionally substituted ar(lower)alkyl, optionally substituted heterocyclic(lower)alkyl or cyclo(lower)alkyl(lower)alkyl, or R₃ and R₄ are linked together to form lower alkylene or condensed ring, or one of R₃ and R₄ is linked to the adjacent nitrogen atom to form a ring; R₅ is lower alkylene or lower alkenylene, Y is [wherein R_{Y1} is hydrogen, halogen or optionally protected hydroxy, R_{Y2} is hydrogen, halogen, lower alkyl or phenyl, and R_{Y3} is hydrogen or lower alkyl]; R₈ is hydrogen or lower alkyl; and n is an integer of 1 or 2, or a salt thereof.

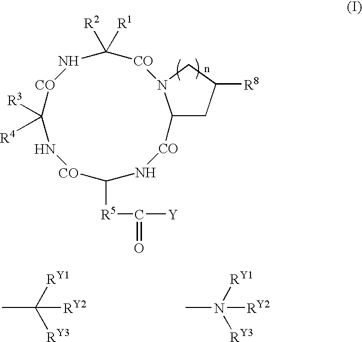

### TECHNICAL FIELD

The present invention relates to a cyclic tetrapeptide compound which is useful as a medicament, to a process for producing the same and to a pharmaceutical composition comprising the same.

### BACKGROUND ART

Histone deacetylases are known to play an essential role in the transcriptional machinery for regulating gene expression, and histone deacetylase inhibitors induce histone hyperacetylation and affect the gene expression. Therefore, a histone deacetylase inhibitor is useful as a therapeutic or prophylactic agent for diseases caused by abnormal gene expression, such as inflammatory disorders, diabetes, diabetic complications, homozygous thalassemia, fibrosis, cirrhosis, acute promyelocytic leukaemia (APL), protozoal infection, and the like.

In this connection, JP-A-7-196686 discloses a cyclic tetrapeptide compound that can be used as an antitumor agent, but this publication is silent on the action against histone deacetylases and the effect against the above-mentioned various diseases.

### SUMMARY OF THE INVENTION

The present invention relates to a novel cyclic tetrapeptide compound which is useful as a medicament, to a process for producing the same and to a pharmaceutical composition comprising the same.

More particularly, the present invention relates to a cyclic tetrapeptide compound which has a potent inhibitory effect on the activity of histone deacetylase.

The inventors of the present invention have also found that a histone deacetylase inhibitor, such as cyclic tetrapeptide compound of formula (I) (hereinafter cyclic tetrapeptide compound [I] or compound [I]), has a potent immunosuppressive effect and potent antitumor effect. Therefore, a histone deacetylase inhibitor, such as cyclic tetrapeptide compound [I], is useful as an active ingredient of an immunosuppressant and an antitumor agent and useful as a therapeutic or prophylactic agent for an organ transplant rejection, autoimmune diseases, tumor, and the like.

Accordingly, one object of the present invention is to provide a compound which has biological activities as stated above.

A further object of the present invention is to provide a pharmaceutical composition containing, as an active ingredient, the cyclic tetrapeptide compound [I].

A yet further object of the present invention is to provide a use of the histone deacetylase inhibitors, such as cyclic tetrapeptide compound [I], for treating and preventing diseases as stated above.

A yet further object of the present invention is to provide a commercial package comprising the pharmaceutical composition containing the cyclic tetrapeptide compound [I] and a written matter associated therewith, the written matter stating that the pharmaceutical composition may or should be used for treating or preventing diseases as stated above.

Thus, the present invention provides a cyclic tetrapeptide compound of the formula (I): 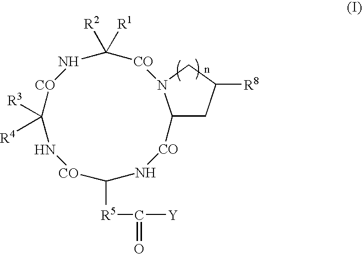
wherein
R¹ is hydrogen,
R² is lower alkyl, aryl, ar(lower)alkyl optionally substituted with one or more suitable substituent(s), heterocyclic(lower)alkyl, cyclo(lower)alkyl(lower)alkyl, lower alkylcarbamoyl(lower)alkyl or arylcarbamoyl(lower)alkyl,
R³ and R⁴ are each independently hydrogen, lower alkyl, ar(lower)alkyl optionally substituted with one or more suitable substituent(s), heterocyclic(lower)alkyl optionally substituted with one or more suitable substituent(s) or cyclo(lower)alkyl(lower)alkyl, or
R³ and R⁴ are linked together to form lower alkylene or condensed ring, or one of R³ and R⁴ is linked to the adjacent nitrogen atom to form a ring,
R⁵ is lower alkylene or lower alkenylene,
Y is 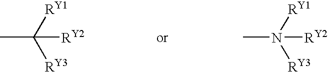
[wherein R^{Y1} is hydrogen, halogen or optionally protected hydroxy,
R^{Y2} is hydrogen, halogen, lower alkyl or phenyl, and
R^{Y3} is hydrogen or lower alkyl],
R⁸ is hydrogen or lower alkyl, and
n is an integer of 1 or 2,
providing that,
when R³ is methyl, R⁴ is methyl or ethyl, R⁵ is pentylene, R⁸ is hydrogen, n is 1, R^{Y1} is optionally substituted hydroxy, R^{Y2} is methyl and R^{Y3} is hydrogen, then R² is not unsubstituted benzyl,
or a salt thereof.

The present invention also provides a cyclic tetrapeptide compound of the formula (I′):
wherein 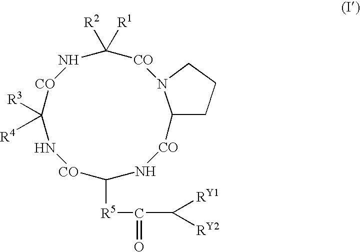
R¹ is hydrogen,
R² is ar(lower)alkyl optionally substituted with one or more suitable substituent(s),
R³ and R⁴ are each hydrogen or lower alkyl, or
R³ and R⁴ are linked together to form lower alkylene,
R⁵ is lower alkylene or lower alkenylene,
R^{Y1} is optionally protected hydroxy, and
R^{Y2} is lower alkyl,
providing that,
when R³ is methyl, R⁴ is methyl or ethyl, R⁵ is pentylene, R^{Y1} is optionally substituted hydroxy and R^{Y2} is methyl, then R² is not unsubstituted benzyl,
or a salt thereof.

### BRIEF DESCRIPTION OF THE DRAWINGS

FIG. 1 represents pNFkB-TA-Luc.

FIG. 2 represents a chart which shows the effect of the compound of the present invention on NF-kB activation in TNFα-stimulated HEL cells (NF-kB reporter gene assay) in comparison with the effect of FK506.

FIG. 3 represents a chart which shows the effect of the compound of the present invention on the MCP-1 production by activated THP-1 cells (MCP-1 ELISA) in comparison with the effect of FK506.

### PREFERRED EMBODIMENT FOR CARRYING OUT THE INVENTION

The compound [I] and a salt thereof can be prepared by the process as illustrated in the following reaction schemes.

The compound [I] of the present invention may be prepared by a liquid phase method (i.e. Preparation A→Preparation C→Examples) or a solid phase-liquid phase relay method (i.e. Preparation B→Preparation C→Examples).

Hereinafter, the processes for preparing the compound [I] of the present invention are explained in detail. 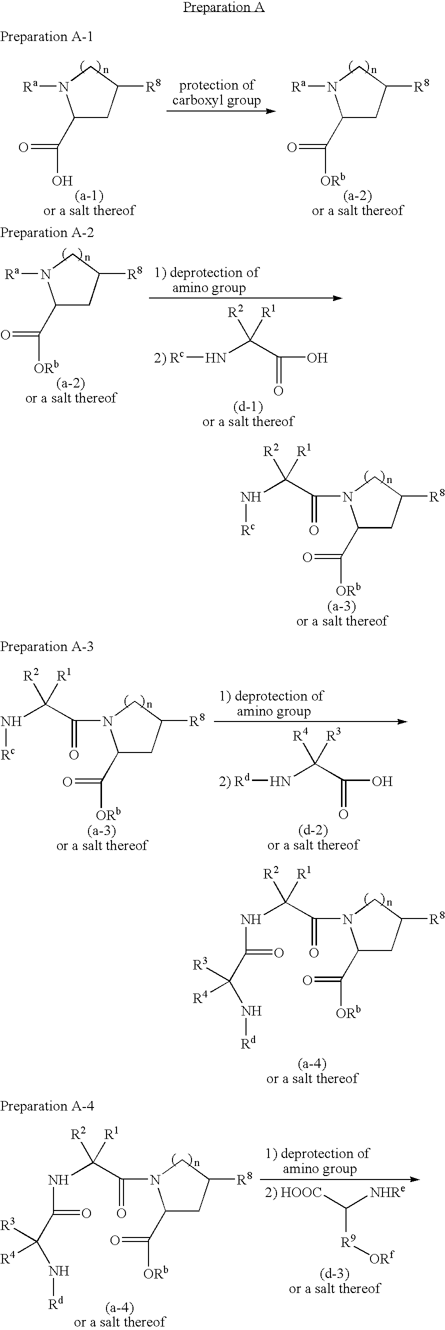 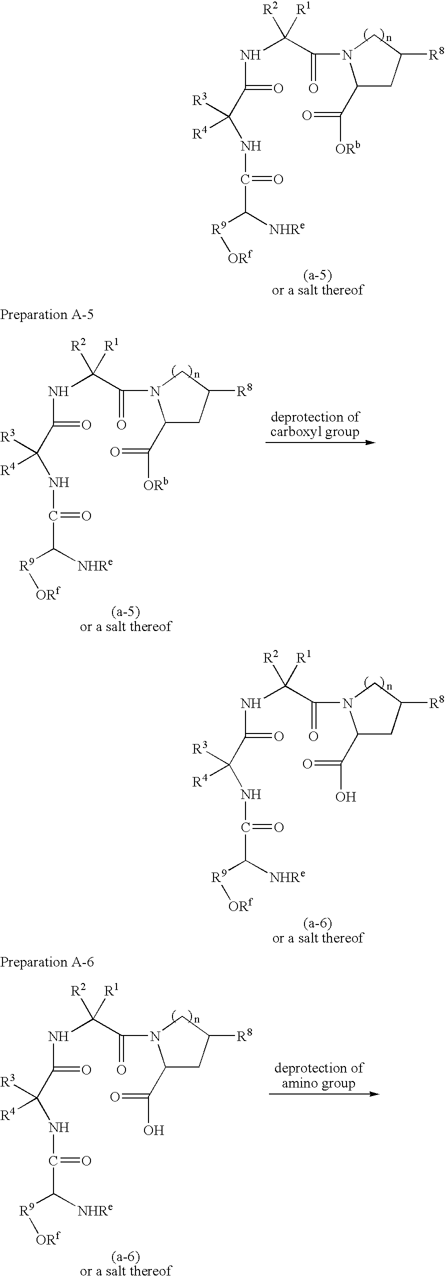 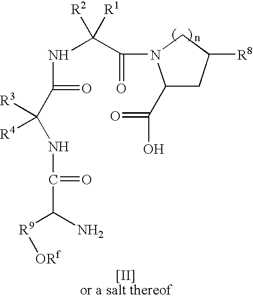
wherein
R¹, R², R³, R⁴, R⁸ and n are as defined above,
R⁹ is lower alkylene,
R^{a} is hydrogen or amino protective group,
R^{b} is carboxy protective group,
R^{c}, R^{d} and R^{e} are each independently amino protective group, and
R^{f} is hydroxy protective group.

In the above Preparation A, the deprotection of carboxyl group is exemplified by Preparation 17 and the like, and the deprotection of amino group is exemplified by Preparation 18 and the like.

Alternatively, the deprotection of carboxyl and amino groups may be conducted simultaneously (e.g. Preparation 53, Preparation 57 and the like). 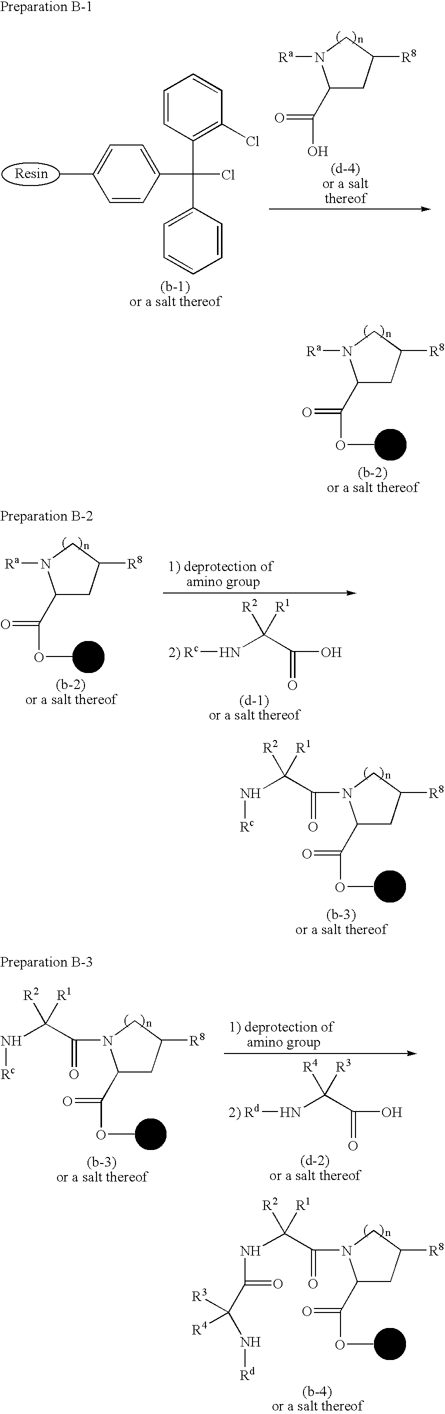 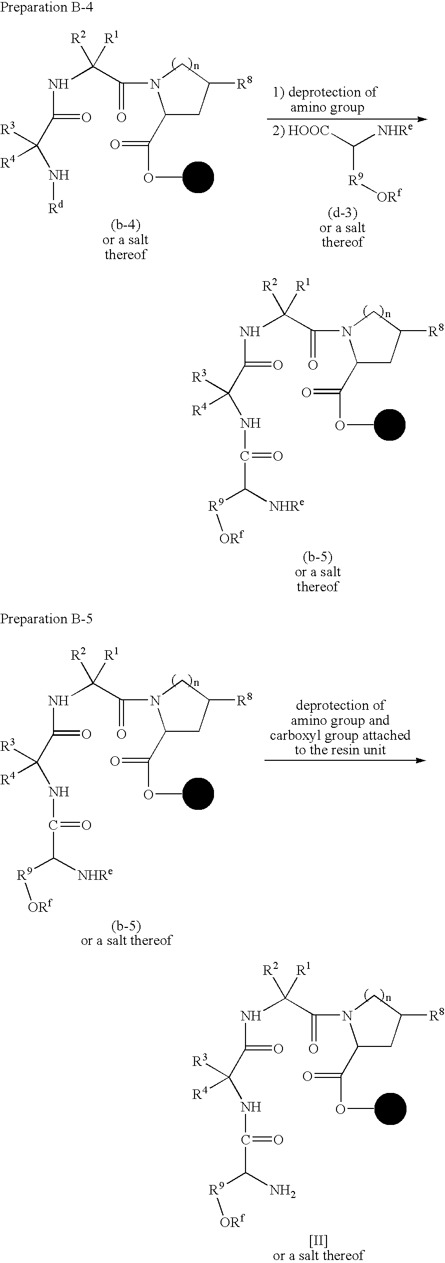
wherein
R¹, R², R³, R⁴, R⁸ and n are as defined above,
R⁹ is lower alkylene,
R^{a} is hydrogen or amino protective group,
R^{c}, R^{d} and R^{e} are each independently amino protective group,
R^{f} is hydroxy protective group, and 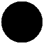
is the following resin unit:

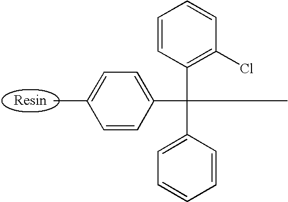

wherein 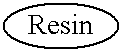
is a resin. 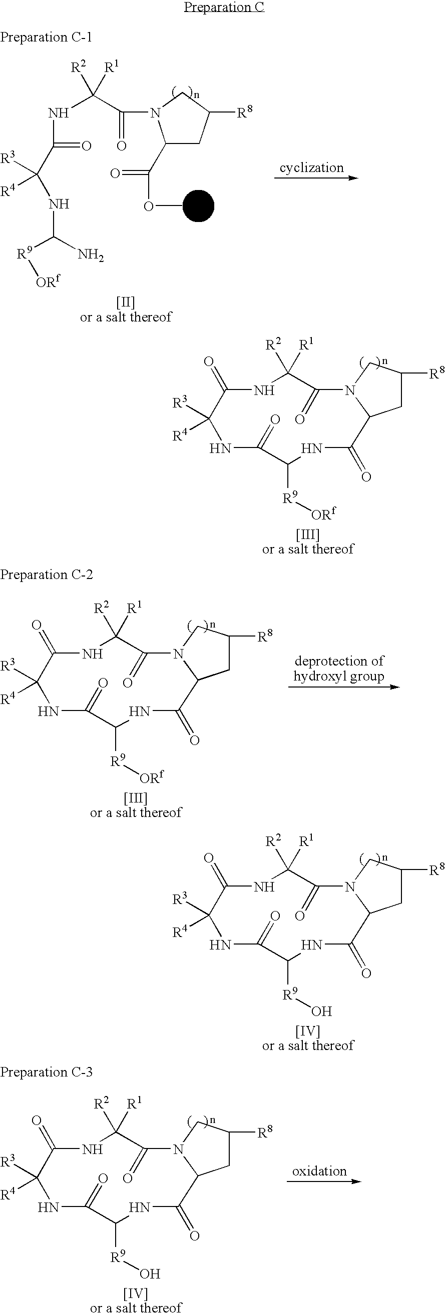 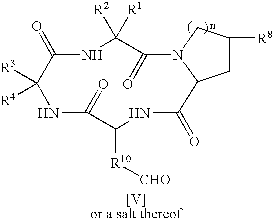
wherein
R¹, R², R³, R⁴, R⁸ and n are as defined above,
R⁹ and R¹⁰ are each independently lower alkylene, and
R^{f} is a hydroxy protective group.

The compound [V] obtained from the Preparation C is used in the preparation of the compound [I] of the present invention.
Preparation of the Compound [I] of the Present Invention 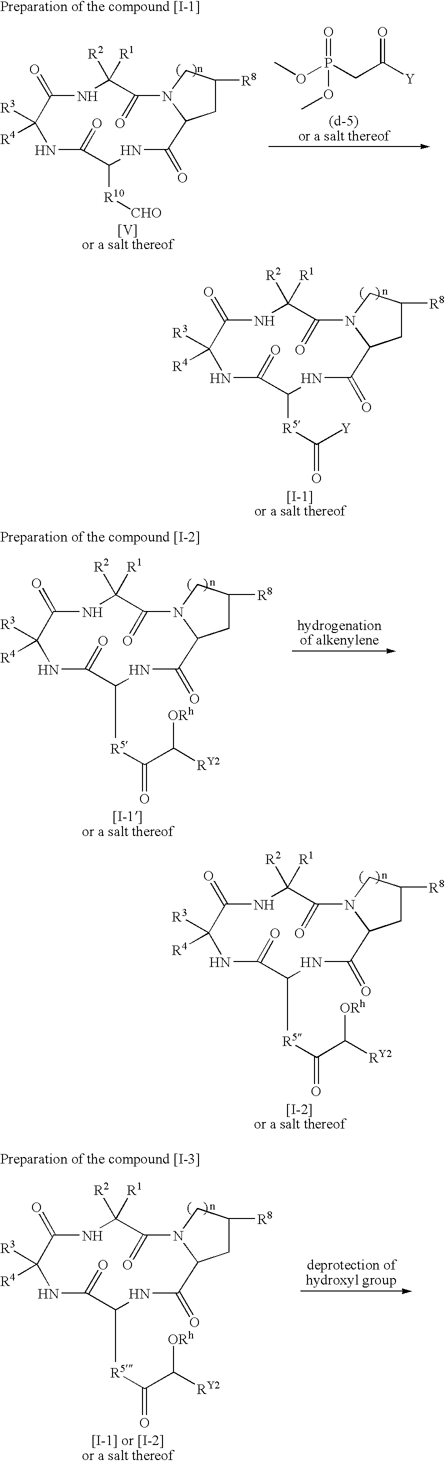 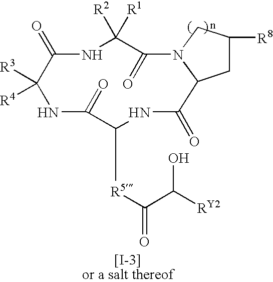
wherein
R¹, R², R³, R⁴, R^{Y2}, R⁸, R⁹ and n are as defined above,
R^{5′} is lower alkenylene,
R^{5″} is lower alkylene,
R^{5′″} is lower alkylene or lower alkenylene, and
R^{h} is hydroxy protective group.

To determine absolute configuration of the hydroxyl group of the compound [I-3] and to estimate optical purity of the isomer of the compound [I-3], the compound [I-3] is reacted with a reagent such as (R or S)-(+ or −)-α-methoxy-α-trifluoromethyl-α-phenylacetyl chloride, 1-naphthylmethoxyacetic acid, 2-naphthylmethoxyacetic acid, 9-anthrylmethoxyacetic acid, 2-anthrylmethoxyacetic acid, and the like. This reaction is exemplified by Example 53.

The hydroxy group of the compound [I-3] is, if desired, optionally protected with a suitable hydroxy protective group. The protection of the hydroxy group is exemplified by Examples 162, 205, 206, 207 and the like. 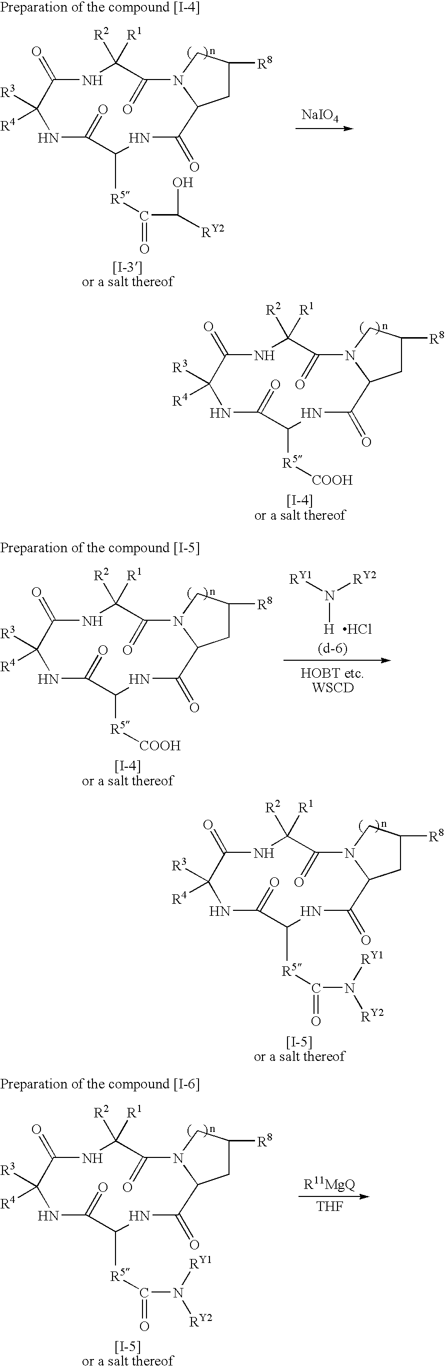 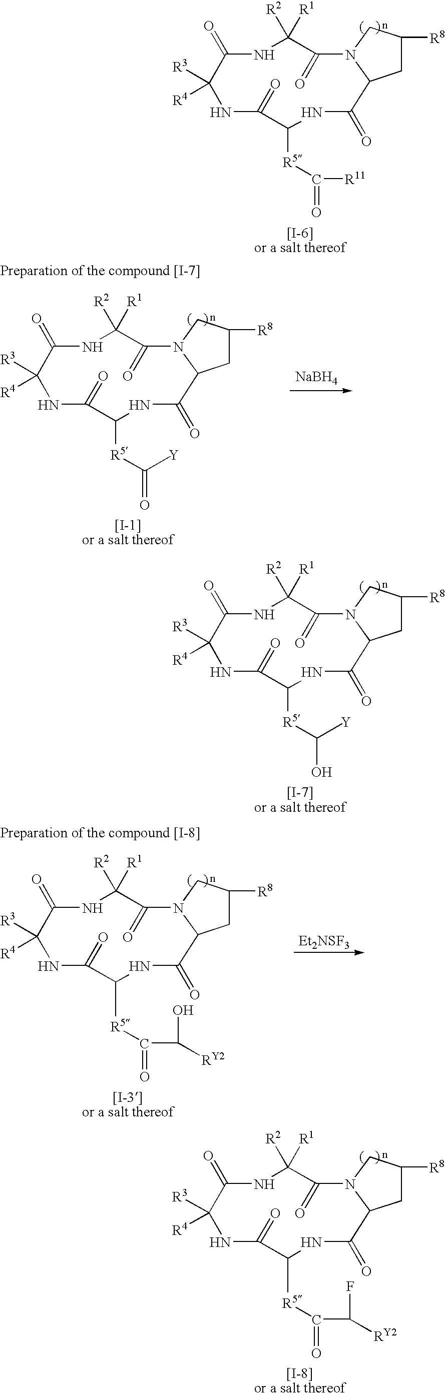 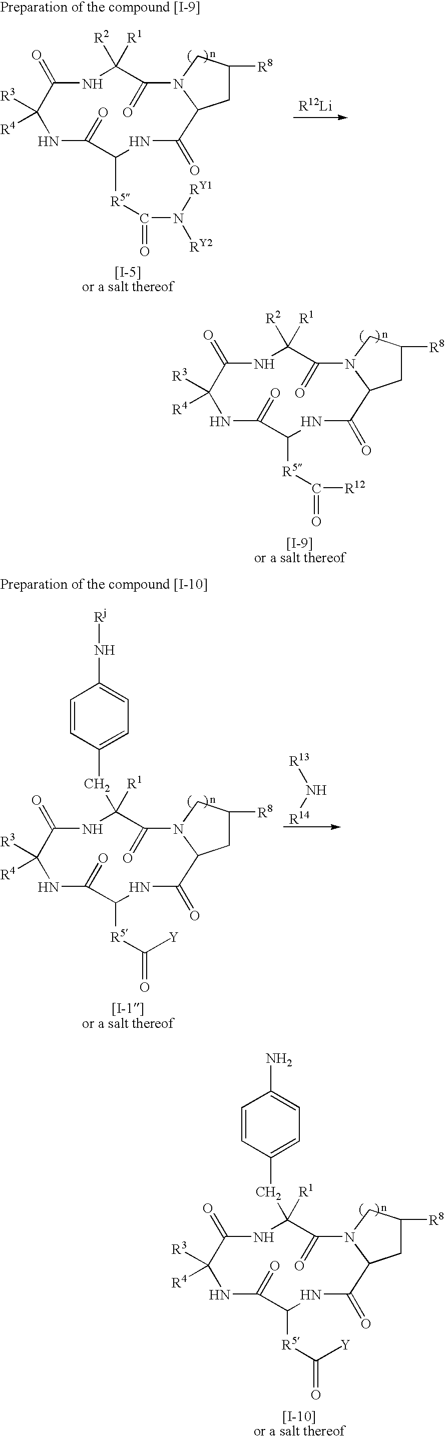 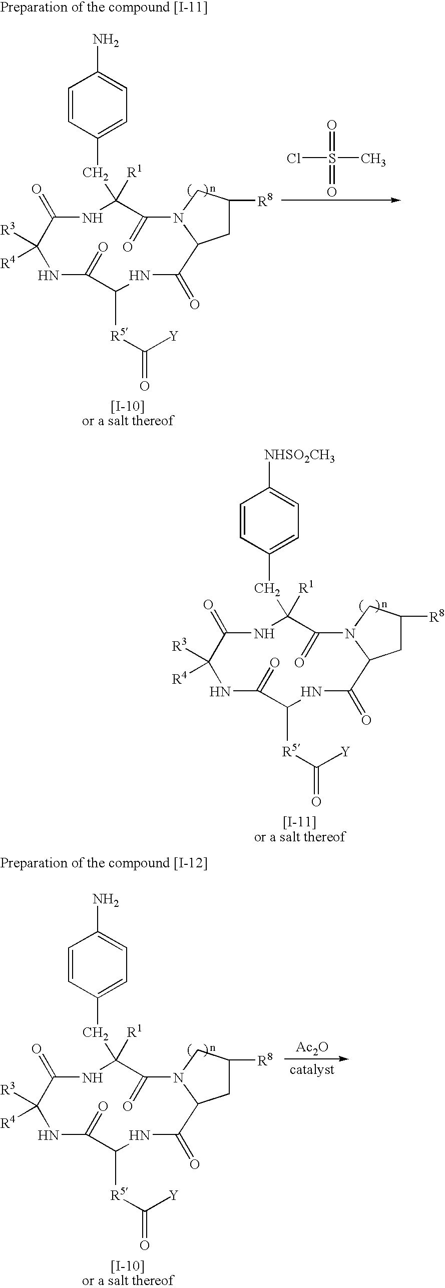 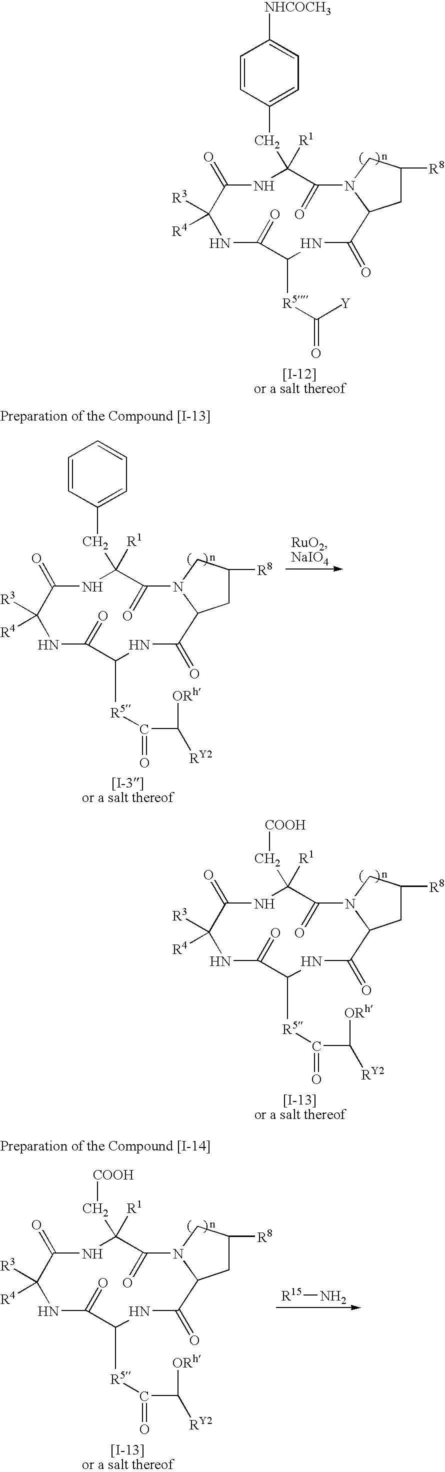 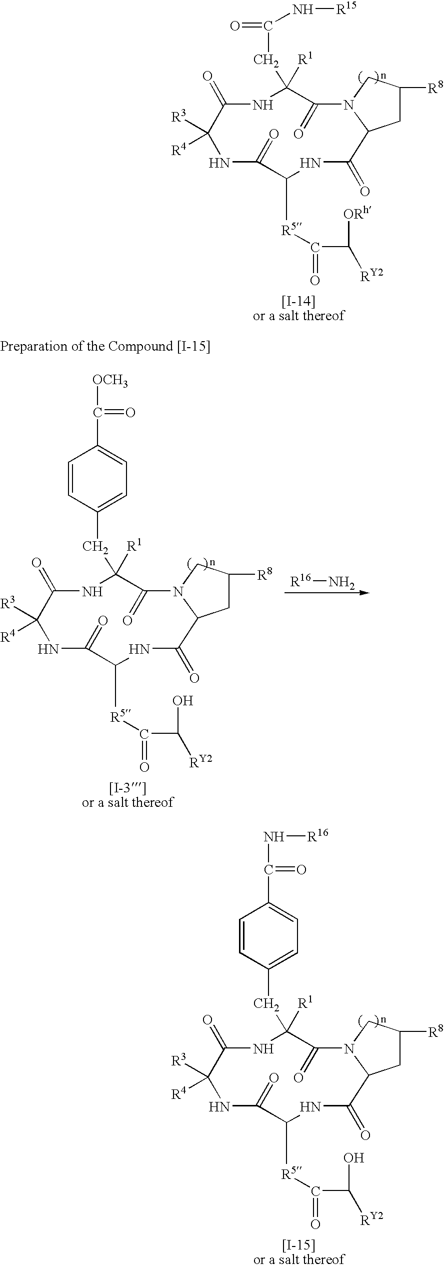
wherein
R¹, R², R³, R⁴, R^{5′}, R^{5″}, R^{5′″}, R^{Y1}, R^{Y2}, R⁸, R⁹, R¹⁰, Y and n are as defined above,
R¹¹ is lower alkyl, aryl or ar(lower)alkyl,
R¹² is lower alkyl, lower alkenyl or aryl and the like,
R¹³ and R¹⁴ are each independently lower alkyl or lower cycloalkyl, or
R¹³ and R¹⁴ are linked together with the adjacent nitrogen atom to form a ring wherein one or more methylene(s) of the ring is (are) optionally replaced by heteroatom(s) selected from an oxygen atom, a nitrogen atom and a sulfur atom,
R¹⁵ is lower alkyl,
R¹⁶ is lower alkyl,
Q is halogen,
R^{h′} is hydroxy protective group, and
R^{j} is amino protective group.

Suitable “salt” is a pharmaceutically acceptable and conventional non-toxic salt, and may include a salt with a base or an acid addition salt such as a salt with an inorganic base, for example, an alkaline metal salt (e.g., sodium salt, potassium salt, and the like), an alkaline earth metal salt (e.g., calcium salt, magnesium salt, and the like), an ammonium salt;

a salt with an organic base, for example, an organic amine salt (e.g., triethylamine salt, diisopropylethylamine salt, pyridine salt, picoline salt, ethanolamine salt, triethanolamine salt, dicyclohexylamine salt, N,N′-dibenzylethylenediamine salt, and the like);

an inorganic acid addition salt (e.g., hydrochloride, hydrobromide, sulfate, phosphate, and the like);

an organic carboxylic sulfonic acid addition salt (e.g., formate, acetate, trifluoroacetate, maleate, tartrate, fumarate, methanesulfonate, benzenesulfonate, toluenesulfonate, and the like); and

a salt with a basic or acidic amino acid (e.g., arginine, aspartic acid, glutamic acid, and the like).

Suitable examples and illustration of the various definitions in the above and subsequent descriptions, which the present invention intends to be included within the scope thereof, are explained in detail as follows:

The term “halogen” means fluorine, chlorine, bromine, and iodine.

The term “lower” used in the description is intended to mean 1 to 6 carbon atoms, unless otherwise indicated.

Suitable example of “one or more” may be the number of 1 to 6, preferably 1 to 3.

Suitable examples of “lower alkyl” may include straight or branched one having 1 to 6 carbon atom(s), such as methyl, ethyl, propyl, isopropyl, butyl, isobutyl, sec-butyl, tert-butyl, pentyl, tert-pentyl, neopentyl, hexyl, isohexyl and the like. The preferred lower alkyl for R² may be 2-methyl-1-propyl, the preferred lower alkyl for R³ and R⁴ may be methyl, ethyl and isopropyl, the preferred lower alkyl for R^{Y2} may be methyl and ethyl, the preferred lower alkyl for R^{Y3} may be methyl and the preferred lower alkyl for R⁸ may be methyl.

Suitable examples of “lower alkylene” may include straight or branched one having 1 to 6 carbon atom(s), such as methylene, ethylene, trimethylene, propylene, tetramethylene, pentamethylene, hexamethylene and the like. The preferred lower alkylene for R³ and R⁴ may be tetramethylene, and the preferred lower alkylene for R⁵ may be pentamethylene.

Suitable examples of “lower alkenylene” may include straight or branched one having 1 to 6 carbon atom(s), such as ethenylene, 1-propenylene, 2-propenylene, 2-methyl-1-propenylene, 2-methyl-2-propenylene, 1-butenylene, 2-butenylene, 3-butenylene, 1-pentenylene, 2-pentenylene, 3-pentenylene, 4-pentenylene, 1-hexenylene, 2-hexenylene, 3-hexenylene, 4-hexenylene, 5-hexenylene and the like, in which the preferred one for R⁵ may be 1-pentenylene.

Suitable examples of “aryl” may include C₆-C₁₆ aryl such as phenyl, naphthyl, anthryl, pyrenyl, phenanthryl, azulenyl and the like, preferably phenyl, naphthyl. The preferred one for R² may be phenyl, and the preferred one for Y may be phenyl.

Suitable examples of ar(lower)alkyl for R² may include phenyl(C₁-C₆)alkyl such as benzyl, phenethyl, phenylpropyl, phenylbutyl, phenylhexyl and the like, naphthyl(C₁-C₆)alkyl such as naphthylmethyl, naphthylethyl, naphthylpropyl, naphthylbutyl, naphthylpentyl, naphtylhexyl and the like. The preferred one for R² may be phenyl(C₁-C₆)alkyl, more preferably benzyl.

Suitable examples of “suitable substituent(s)” of “ar(lower)alkyl optionally substituted with one or more suitable substituent(s)” for R² may include lower alkyl (e.g. methyl and the like), halo(lower)alkyl (e.g. trifluoromethyl and the like) lower alkoxy (e.g. methoxy, ethoxy and the like), ar(lower)alkoxy (e.g. phenyl(lower)alkoxy and the like), cyano, hydroxy, halogen (e.g. chloro, fluoro and the like), amino, lower alkanoylamino (e.g. acetylamino and the like), lower alkylsulfonylamino (e.g. methanesulfonylamino and the like), aryl (e.g. phenyl and the like), cyclo(lower)alkyloxy (e.g. cyclopentyloxy and the like), carboxy(lower)alkoxy (e.g. carboxymethoxy and the like), heterocyclic(lower)alkoxy (e.g. pyridyl(lower)alkoxy such as pyridylmethoxy and the like), lower alkenyloxy (e.g. ethenyloxy and the like), hydroxy(lower)alkyl (e.g. hydroxymethyl and the like), arylcarbamoyl (e.g. phenylcarbamoyl and the like), heterocycliccarbonyl (e.g. piperidinocarbonyl and the like), lower(alkyl)carbamoyl(lower)alkoxy (e.g. n-pentylcarbamoylmethoxy and the like), arylcarbamoyl(lower)alkoxy (e.g., phenylcarbamoyl(lower)alkoxy such as phenylcarbamoylmethoxy and the like), lower(alkyl)carbamoyl(lower)alkyl (e.g. 2-(t-butylcarbamoyl)-1-ethyl and the like), heterocyclic group (e.g. pyridyl and the like), lower alkoxycarbonyl (e.g. methoxycarbonyl and the like), lower alkoxycarbonyl(lower)alkoxy (e.g. methoxycarbonylmethoxy and the like), lower alkylcarbamoyl (e.g. methylcarbamoyl and the like), heterocycliccarbonyl(lower)alkyl (e.g. morpholinocarbonyl(lower)alkyl such as 2-morpholinocarbonyl-1-ethyl and the like), heterocycliccarbonyl(lower)alkoxy (e.g. piperidinocarbonyl(lower)alkoxy such as piperidinocarbonylmethoxy and the like), aryl(lower)alkoxy (e.g. phenyl(lower)alkoxy such as phenylmethoxy and the like) and arylcarbamoyl(lower)alkyl (e.g. phenylcarbamoyl(lower)alkyl such as phenylcarbamoylmethyl and the like) and the like.

Suitable “heterocyclic” in the terms of “heterocyclic(lower)alkyl” for R² may include 5- or 6-membered heteromonocyclic group or condensed heterocyclic group, each of which contains at least one heteroatom(s) selected from a sulfur atom, an oxygen atom and a nitrogen atom.

Suitable 5- or 6-membered heteromonocyclic group containing at least one heteroatom(s) selected from a sulfur atom, an oxygen atom and a nitrogen atom include, for example, pyridyl, dihydropyridyl, azepinyl (e.g., 1H-azepinyl and the like), pyrrolyl, pyrrolinyl, imidazolyl, pyrazolyl, pyrimidinyl, pyrazinyl, pyridazinyl, triazolyl (e.g., 4H-1,2,4-triazolyl, 1H-1,2,3-triazolyl, 2H-1,2,3-triazolyl and the like), tetrazolyl (e.g., 1H-tetrazolyl, 2H-tetrazolyl and the like), perhydroazepinyl (e.g., perhydro-1H-azepinyl and the like), pyrrolidinyl, imidazolidinyl, piperidyl, piperadinyl, oxazolyl, isoxazolyl, oxadiazolyl (e.g., 1,2,4-oxadiazolyl, 1,3,4-oxadiazolyl, 1,2,5-oxadiazolyl and the like), morpholinyl, sydnonyl, thiazolyl, isothiazolyl, thiadiazolyl (e.g., 1,2,3-thiazidiazolyl, 1,2,4-thiadiazolyl, 1,3,4-thiadiazolyl, 1,2,5-thiadiazolyl and the like), dihydrothiazinyl, thiazolidinyl, furyl, di-hydrooxatiinyl and the like.

Suitable condensed heterocyclic group containing at least one heteroatom(s) selected from a sulfur atom, an oxygen atom and a nitrogen atom include, for example, indolyl, isoindolyl, indolidinyl, benzimidazolyl, quinolyl, isoquinolyl, indazolyl, benzotriazolyl, quinoxalinyl, imidazopyridyl (e.g., imidazo[4,5-c]pyridyl and the like), tetrahydroimidazopyridyl (e.g., 4,5,6,7-tetrahydro[4,5-c]pyridyl and the like), 7-azabicyclo[2.2.1]heptyl, 3-azabicyclo[3.2.2]nonanyl, benzoxazolyl, benzoxadiazolyl, benzothiazolyl, benzothiadiazolyl, benzothienyl, benzodithiinyl, benzoxathiinyl and the like.

Among these, the preferable “heterocyclic” in the terms of “heterocyclic(lower)alkyl” for R² include, for example, pyridyl, pyrazinyl, pyrimidinyl, pyridazinyl, thienyl, furyl, quinolyl, imidazolyl, indolyl and the like. The preferred “heterocyclic(lower)alkyl” for R² may be 2-pyridylmethyl, 4-pyridylmethyl, 3-indolylmethyl and the like.

Suitable “cyclo(lower)alkyl” moiety in the terms of “cyclo(lower)alkyl(lower)alkyl” for R² may be cyclopropyl, cyclobutyl, cyclopentyl, cyclohexyl, and the like. The preferred “cyclo(lower)alkyl(lower)alkyl” for R² may be cyclopropylmethyl, cyclobutylmethyl, cyclopentylmethyl, cyclohexylmethyl, cyclopropylethyl, cyclobutylethyl, cyclopentylethyl, cyclohexylethyl and the like.

Suitable example of “lower alkylcarbamoyl(lower)alkyl” for R² may include n-pentylcarbamoylmethyl and the like.

Suitable example of “arylcarbamoyl(lower)alkyl” for R² may include phenylcarbamoylmethyl and the like.

Suitable “ar(lower)alkyl” for R³ and R⁴ may include phenyl(lower)alkyl [e.g. phenyl(C₁-C₆)alkyl such as benzyl, phenethyl, phenylpropyl, phenylbutyl, phenylhexyl and the like], naphthyl(lower)alkyl [e.g. naphthyl(C₁-C₆)alkyl such as naphthylmethyl, naphthylethyl, naphthylpropyl, naphthylbutyl, naphthylhexyl and the like], and the like. The preferred one for R³ and R⁴ may be phenyl(C₁-C₆)alkyl, more preferably benzyl.

Suitable example of “suitable substituent(s)” of “ar(lower)alkyl optionally substituted with one or more suitable substituent(s)” for R³ and R⁴ may include lower alkoxy, lower alkyl, cyano, halogen, amino, nitro, carboxy and the like. The preferred “ar(lower)alkyl optionally substituted with one or more suitable substituent(s)” for R³ and R⁴ may include (4-methoxyphenyl)methyl, (4-ethoxyphenyl)methyl and the like.

Suitable “heterocyclic(lower)alkyl” for R³ and R⁴ may include, for example, indenylmethyl, pyridylmethyl, thienylmethyl, furylmethyl, imidazolylmethyl and the like.

Suitable example of “suitable substituent(s)” of “heterocyclic(lower)alkyl optionally substituted with one or more suitable substituent(s)” for R³ and R⁴ may be methyl, ethyl, alkoxy, cyano, halogen and the like, and the preferred “heterocyclic(lower)alkyl optionally substituted with one or more suitable substituent(s)” for R³ and R⁴ may include N-methyl-2-indenylmethyl and the like.

Suitable example of “cyclo(lower)alkyl(lower)alkyl” for R³ and R⁴ may be cyclohexylmethyl, cyclopentylmethyl and the like.

Suitable example of “condensed ring” for R³ and R⁴ may be, for example, 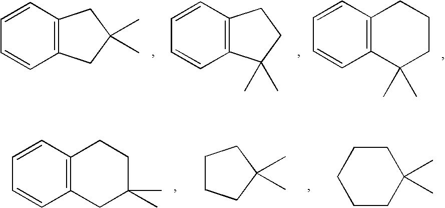
and the like.

Suitable example of the “ring” of the “one of R³ and R⁴ is linked to the adjacent nitrogen atom to form a ring” may be, for example, 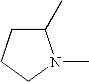
and the like.

Suitable “lower alkyl” for R¹¹ may be methyl, ethyl and the like, suitable “aryl” for R¹¹ may be C₆-C₁₂ aryl such as phenyl and the like, and suitable “ar(lower)alkyl” for R¹¹ may be (C₆-C₁₂)aryl(C₁-C₆)alkyl such as benzyl and the like.

Suitable “lower alkyl” for R¹² may be methyl, ethyl, propyl (e.g., isopropyl and the like), butyl (e.g., isobutyl, t-butyl and the like), hexyl (e.g., n-hexyl) and the like, suitable “lower alkenyl” for R¹² may be vinyl and the like, and suitable “aryl” for R¹² may be C₆-C₁₂ aryl such as phenyl and the like.

Suitable “lower alkyl” for R¹³ and R¹⁴ may be lower alkyl (e.g., methyl, ethyl and the like) and suitable “lower cycloalkyl” for R¹³ and R¹⁴ may be cyclohexyl and the like.

Suitable “ring” of the “ring wherein one or more methylene(s) of the ring is (are) optionally replaced by heteroatom(s) selected from an oxygen atom, a nitrogen atom and a sulfur atom” for R¹³ and R¹⁴ may be piperidino, morpholino and the like.

Suitable “lower alkyl” for R¹⁵ may be lower alkyl. The preferred one for R¹⁵ may be pentyl.

Suitable “lower alkyl” for R¹⁶ may be lower alkyl. The preferred one for R¹⁶ may be methyl.

Suitable carboxy protective group may include:

lower alkyl (e.g. methyl, ethyl, propyl, isopropyl, butyl, isobutyl, t-butyl, pentyl, hexyl and the like), preferably methyl, ethyl and t-butyl;

mono(or di or tri)halo(lower)alkyl (e.g. 2-iodoethyl, 2,2,2-trichloroethyl and the like), preferably 2,2,2-trichloroethyl;

lower alkanoyloxy(lower)alkyl (e.g. acetoxymethyl, propionyloxymethyl, butyryloxymethyl, valeryloxymethyl, pivaloyloxymethyl, hexanoyloxymethyl, 1(or 2)-acetoxyethyl, 1(or 2 or 3)-acetoxypropyl, 1(or 2 or 3 or 4)-acetoxybutyl, 1(or 2)-propionyloxyethyl, 1(or 2 or 3)-propionyloxypropyl, 1(or 2)-butyryloxyethyl, 1(or 2)-isobutyryloxyethyl, 1(or 2)-pivaloyloxyethyl, 1(or 2)-hexanoyloxyethyl, isobutyryloxymethyl, 2-ethylbutyryloxymethyl, 3,3-dimethylbutyryloxymethyl, 1(or 2)-pentanoyloxyethyl, and the like);

lower alkanesulfonyl(lower)alkyl (e.g. 2-mesylethyl and the like);

lower alkoxycarbonyloxy(lower)alkyl (e.g. methoxycarbonyloxymethyl, ethoxycarbonyloxymethyl, 2-methoxycarbonyloxyethyl, 1-ethoxycarbonyloxyethyl, 1-isopropoxycarbonyloxyethyl, and the like);

[5-(lower)alkyl-2-oxo-1,3-dioxol-4-yl](lower)alkyl (e.g. (5-methyl-2-oxo-1,3-dioxol-4-yl)methyl, (5-ethyl-2-oxo-1,3-dioxol-4-yl)methyl, (5-propyl-2-oxo-1,3-dioxol-4-yl)methyl, and the like);

aryl optionally substituted with one or more suitable substituent(s) (e.g. phenyl, o(or m or p)-chlorophenyl, tolyl, o(or m or p)-t-butylphenyl, xylyl, mesityl, cumenyl, and the like);

ar(lower)alkyl in which the aryl portion is optionally substituted with one or more suitable substituent(s) (e.g. benzyl, p-methoxybenzyl, o(or p)-nitrobenzyl, phenethyl, trityl, benzhydryl, bis(methoxyphenyl)methyl, m,p-dimethoxybenzyl, 4-hydroxy-3,5-di-t-butylbenzyl, and the like), preferably benzyl, p-methoxybenzyl and o(or p)-nitrobenzyl;

arylcarbonyl(lower)alkyl in which the aryl portion is optionally substituted with one or more suitable substituent(s) (e.g. phenacyl and the like);

cyclo(lower)alkyl (e.g. cyclopropyl, cyclobutyl, cyclopentyl, cyclohexyl, and the like);

lower alkenyl (e.g. vinyl, allyl and the like), preferably allyl;

lower alkynyl (e.g. ethynyl, propynyl, and the like);

trisubstituted silyl such as tri(lower)alkylsilyl (e.g. trimethylsilyl, triethylsilyl, tributylsilyl, tert-butyldimethylsilyl, tri-tert-butylsilyl, and the like), lower alkyldiarylsilyl (e.g. methyldiphenylsilyl, ethyldiphenylsilyl, propyldiphenylsilyl, tert-butyldiphenylsilyl, and the like), and the like, preferably trimethylsilyl, triethylsilyl, tert-butyldimethylsilyl and tert-butyldiphenylsilyl;

tri(lower)alkylsilyl(lower)alkyl (e.g. 2-(trimethylsilyl)ethyl and the like);

1-(lower)alkyl-2,6,7-trioxabicyclo[2.2.2]oct-4-yl (e.g. 1-methyl-2,6,7-trioxabicyclo[2.2.2]oct-4-yl, 1-ethyl-2,6,7-trioxabicyclo[2.2.2]oct-4-yl, and the like); and the like.

Suitable hydroxy protective group may include:

lower alkyl such as methyl, ethyl, propyl, isopropyl, butyl, isobutyl, t-butyl, pentyl, hexyl, and the like, preferably methyl;

lower alkoxy(lower)alkyl (e.g. methoxymethyl and the like);

lower alkoxy(lower)alkoxy(lower)alkyl (e.g. 2-methoxyethoxymethyl and the like);

ar(lower)alkyl in which the aryl portion is optionally substituted with one or more suitable substituent(s) (e.g. benzyl (Bn), p-methoxybenzyl, m,p-dimethoxybenzyl, and the like), preferably benzyl; ar(lower)alkoxy(lower)alkyl in which the aryl portion is optionally substituted with one or more suitable substituent(s) (e.g. benzyloxymethyl, p-methoxybenzyloxymethyl, and the like);

(lower)alkylthio(lower)alkyl (e.g. methylthiomethyl, ethylthiomethyl, propylthiomethyl, isopropylthiomethyl, butylthiomethyl, isobutylthiomethyl, hexylthiomethyl, and the like), and the like, preferably methylthiomethyl;

heterocyclic group (e.g. tetrahydropyranyl and the like);

trisubstituted silyl such as tri(lower)alkylsilyl (e.g. trimethylsilyl, triethylsilyl, tributylsilyl, tert-butyldimethylsilyl, tri-tert-butylsilyl, and the like), lower alkyldiarylsilyl (e.g. methyldiphenylsilyl, ethyldiphenylsilyl, propyldiphenylsilyl, tert-butyldiphenylsilyl (TBDPS), and the like), and the like, preferably tert-butyldimethylsilyl (TBDMS) and tert-butyldiphenylsilyl;

acyl as described below [e.g. aliphatic acyl such as lower alkanoyl (e.g. acetyl, propanoyl, pivaloyl, and the like); aromatic acyl (e.g. benzoyl (Bz), toluoyl, naphthoyl, fluorenylcarbonyl and the like);

lower alkoxycarbonyl (e.g. methoxycarbonyl, ethoxycarbonyl, propoxycarbonyl, isopropoxycarbonyl, butoxycarbonyl, isobutoxycarbonyl, t-butoxycarbonyl, pentyloxycarbonyl, hexyloxycarbonyl, and the like), and the like;

ar(lower)alkoxycarbonyl in which the aryl portion is optionally substituted with one or more suitable substituent(s) (e.g. benzyloxycarbonyl, bromobenzyloxycarbonyl and the like);

lower alkylsulfonyl (e.g. methylsulfonyl, ethylsulfonyl, and the like);

lower alkoxysulfonyl (e.g. methoxysulfonyl, ethoxysulfonyl, and the like);

ar(lower)alkanoyl (e.g. phenylacetyl, phenylpropanoyl, phenylbutanoyl, phenylisobutanoyl, phenylpentanoyl, phenylhexanoyl, naphthylacetyl, naphthylpropanoyl, naphthylbutanoyl, naphthylisobutanoyl, naphthylpentanoyl, naphthylhexanoyl, and the like);

ar(lower)alkenoyl such as ar(C₃-C₆)alkenoyl (e.g. phenylpropenoyl, phenylbutenoyl, phenylmethacryloyl, phenylpentenoyl, phenylhexenoyl, naphthylpropenoyl, naphthylbutenoyl, naphthylmethacryloyl, naphthylpentenoyl, naphthylhexenoyl, and the like); and the like];

lower alkenyl (e.g. vinyl, allyl, and the like), preferably allyl;

tetrahydropyranyl; and the like.

Suitable “amino protective group” may include:

acyl as exemplified for the hydroxy protective group;

ar(lower)alkyl in which the aryl portion is optionally substituted with one or more suitable substituent(s) (e.g. benzyl, p-methoxybenzyl, o(or p)-nitrobenzyl, phenethyl, trityl, benzhydryl, bis(methoxyphenyl)methyl, m,p-dimethoxybenzyl, 4-hydroxy-3,5-di-t-butylbenzyl, and the like);

[5-(lower)alkyl-2-oxo-1,3-dioxol-4-yl](lower)alkyl (e.g. (5-methyl-2-oxo-1,3-dioxol-4-yl)methyl, (5-ethyl-2-oxo-1,3-dioxol-4-yl)methyl, (5-propyl-2-oxo-1,3-dioxol-4-yl)methyl, and the like), and the like; and the like.

Suitable “acyl” for the present invention may be illustrated as follows:

aliphatic acyl such as alkanoyl (e.g., formyl, acetyl, propanoyl, butanoyl, 2-methylpropanoyl, pentanoyl, pivaloyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl, icosanoyl, and the like);

alkoxycarbonyl (e.g., methoxycarbonyl, ethoxycarbonyl, propoxycarbonyl, isopropoxycarbonyl, butoxycarbonyl, t-butoxycarbonyl, pentyloxycarbonyl, heptyloxycarbonyl, and the like);

alkylsulfonyl (e.g., methylsulfonyl, ethylsulfonyl, and the like);

alkoxysulfonyl (e.g., methoxysulfonyl, ethoxysulfonyl, and the like);

and the like;

aromatic acyl such as aroyl (e.g., benzoyl, toluoyl, naphthoyl, fluorenylcarbonyl, and the like);

ar(lower)alkanoyl such as phenyl(lower)alkanoyl (e.g., phenylacetyl, phenylpropanoyl, phenylbutanoyl, phenylisobutanoyl, phenylpentanoyl, phenylhexanoyl, and the like), naphthyl(lower)alkanoyl (e.g., naphthylacetyl, naphthylpropanoyl, naphthylbutanoyl, and the like), and the like;

ar(lower)alkenoyl such as ar(C₃-C₆)alkenoyl (e.g., phenylpropenoyl, phenylbutenoyl, phenylmethacryloyl, phenylpentenoyl, phenylhexenoyl, and the like),

naphthyl (C₃-C₆)alkenoyl (e.g., naphthylpropenoyl, naphthylbutenoyl, naphthylmethacryloyl, naphthylpentenoyl, naphthylhexenoyl, and the like), and the like;

ar(lower)alkoxycarbonyl such as phenyl(lower)alkoxycarbonyl (e.g., benzyloxycarbonyl, and the like), fluorenyl(lower)alkoxycarbonyl (e.g., fluorenylmethyloxycarbonyl, and the like), and the like;

aryloxycarbonyl (e.g., phenoxycarbonyl, naphthyloxycarbonyl, and the like);

aryloxy(lower)alkanoyl (e.g., phenoxyacetyl, phenoxypropionyl, and the like);

arylcarbamoyl (e.g., phenylcarbamoyl and the like);

arylthiocarbamoyl (e.g., phenylthiocarbamoyl and the like);

arylglyoxyloyl (e.g., phenylglyoxyloyl, naphthylglyoxyloyl, and the like);

arylsulfonyl in which the aryl portion is optionally substituted with one or more suitable substituent(s) (e.g., phenylsulfonyl, p-tolylsulfonyl, and the like);

heterocyclic acyl (e.g. heterocycliccarbonyl and the like);

heterocyclic(lower)alkanoyl (e.g., heterocyclicacetyl, heterocyclicpropanoyl, heterocyclicbutanoyl, heterocyclicpentanoyl, heterocyclichexanoyl, and the like); heterocyclic (lower)alkenoyl (e.g., heterocyclicpropenoyl, heterocyclicbutenoyl, heterocyclicpentenoyl, heterocyclichexenoyl, and the like); heterocyclicglyoxyloyl; and the like.

Suitable “heterocyclic” moiety in the terms “heterocycliccarbonyl”, “heterocyclic(lower)alkanoyl”, heterocyclic(lower)alkenoyl” and “heterocyclicglyoxyloyl” is the same as the above-mentioned “heterocyclic” for the “heterocyclic(lower)alkyl” for R².

Any “resin” known in the field of peptide synthesis may be used for the synthesis of the compound [I] of the present invention. Suitable example of the “resin” for the synthesis of the compound [I] includes 2-chlorotrityl resin and the like.

When the compound [I] has stereoisomers, such isomers are also encompassed in the present invention.

The compound [I] may form a salt, which is also encompassed in the present invention. For example, when a basic group such as an amino group is present in a molecule, the salt is exemplified by an acid addition salt (e.g. salt with an inorganic acid such as hydrochloric acid, hydrobromic acid, sulfuric acid, and the like, salt with an organic acid such as methanesulfonic acid, fumaric acid, maleic acid, mandelic acid, citric acid, salicylic acid, and the like) is exemplified, and when an acidic group such as carboxyl group is present, a basic salt (e.g. salt with a metal such as sodium, potassium, calcium, magnesium, aluminium, and the like, a salt with amino acid such as lysine, and the like), and the like.

In addition, solvates of the compound [I] such as hydrate, ethanolate, and the like, are also encompassed in the present invention.

Hereinafter the reactions in each Preparations and Examples for preparing the cyclic tetrapeptide compound [I] of the present invention are explained in more detail. The invention should not be restricted by the following Preparations and Examples in any way.

Preparation A

Preparation A-1

The compound (a-2) can be prepared by protecting the carboxyl group of the compound (a-1).

Suitable protective agent for the reaction may be, for example, benzylhalide (e.g. benzylbromide and the like), methyl iodide, ethyl iodide, substituted benzyl halide, and the like.

The reaction may be carried out in the presence of a base (e.g. cesium carbonate, potassium carbonate, sodium carbonate, sodium bicarbonate, triethylamine, 1,8-diazabicyclo[5.4.0]undec-7-ene (DBU), pyridine, and the like).

The reaction may be carried out in a conventional solvent which does not adversely influence the reaction (e.g. N,N-dimethylformamide, N-methylpyrrolidone, tetrahydrofuran, dimethylsulfoxide, and the like).

The reaction temperature is not critical and the reaction is usually carried out from under cooling to heating.

This Preparation is exemplified by Preparation 13 and the like.

Preparation A-2

The compound (a-3) can be prepared by 1) deprotecting the amino group of the compound (a-2) and 2) reacting the compound (a-2) with the compound (d-1).

1) Deprotection of the Amino Group of the Compound (a-2)

Suitable deprotective agent for the reaction may be, for example, hydrogen chloride in suitable solvents (such as ethyl acetate, 1,4-dioxane, methanol, ethanol, and the like), trifluoroacetic acid, N,N-diethylamine, and the like. The deprotection may also be conducted with a hydrogenolysis catalyst (e.g. palladium on carbon (Pd—C), palladium hydroxide on carbon, and the like) under hydrogen atmosphere. Specifically, when the carboxylprotective group of the compound (a-2) is t-butyl (e.g. Compound (47)) and the like, the reaction is carried out in the presence of the above-mentioned hydrogenolysis catalyst under hydrogen atmosphere.

The reaction may be carried out in a conventional solvent which does not adversely influence the reaction (e.g. ethyl acetate, dioxane, dichloromethane, acetonitrile, methanol, ethanol, tetrahydrofuran, acetic acid, and the like). Specifically, when trifluoroacetic acid is used as a deprotective agent, the reaction is generally carried out in dichloromethane or without solvent (neat).

The temperature of the reaction is not critical and the reaction is usually carried out from under cooling to heating under the pressure of 1-5 atm.

Alternatively, the compound (a-2) in which the amino group is not protected, may be obtained by directly protecting the carboxyl group of D-proline, in substantially the same manner as Preparation A-1.

2) Reaction of the Compound (a-2) with the Compound (d-1)

The reaction may be carried out in the presence of carbodiimide [e.g. 1-ethyl-3-(3′-N,N-dimethylaminopropyl)-carbodiimide (EDC) or hydrochrolide thereof, dicyclohexylcarbodiimide (DCC), and the like], benzotriazol-1-yloxy-tris-pyrrolidinophosphonium hexafluorophosphate (PyBOP®), benzotriazol-1-yloxy-tris-(dimethylamino)phosphonium hexafluoro phosphate (BOP), bromo-tris-pyrrolidinophosphonium hexafluorophosphate (PyBroP®), 1,1′-carbonyldiimidazol (CDI), diphenylphosphoryl azide (DPPA), 1-hydroxybenzotriazole (HOBT), benzotriazol-1-yloxy-tris-(dimethylamino)phosphoniumhexafluorophosphate (HATU), 2-(1H-benzotriazol-1-yl)-1,1,3,3-tetramethyluromium tetrafluoroborate (TBTU), 2-(1H-benzotriazole-1-yl)-1,1,3,3-tetramethyluronium hexafluorophosphate (HBTU), and the like, and a base [e.g. Hünig base (e.g. N,N-diisopropylethylamine, triethylamine, and the like), and the like], and the like.

The reaction may be carried out in a conventional solvent which does not adversely influence the reaction (e.g. dichloromethane, N,N-dimethylformamide, and the like).

The temperature of the reaction is not critical and the reaction is usually carried out from under cooling to heating.

This Preparation is exemplified by Preparation 14 and the like.

Preparation A-3

The compound (a-4) can be prepared by 1) deprotecting the amino group of the compound (a-3) and 2) reacting the compound (a-3) with the compound (d-2).

1) Deprotection of the Amino Group of the Compound (a-3)

The reaction may be carried out in substantially the same manner as described above for the deprotection of the amino group of the compound (a-2) in the Preparation A-2. Specifically, when the amino protective group is fluorenylmethyloxycarbonyl (Fmoc), a base such as N,N-diethylamine, piperidine, morpholine, dicyclohexylamine, 4-dimethylaminopyridine, N,N-diisopropylethyl amine and the like is used as a deprotective agent, and the reaction is generally carried out in a solvent such as N,N-dimethylformamide, acetonitrile, dichloromethane, and the like, or without solvent (neat).

2) Reaction of the Compound (a-3) with the Compound (d-2)

The reaction may be carried out in substantially the same manner as described above for the reaction of the compound (a-2) with the compound (d-1) in the Preparation A-2.

This Preparation is exemplified by Preparation 15 and the like.

Preparation A-4

The compound (a-5) can be prepared by 1) deprotecting the amino group of the compound (a-4) and 2) reacting the compound (a-4) with the compound (d-3).

1) Deprotection of the Amino Group of the Compound (a-4)

The reaction may be carried out in substantially the same manner as described above for the deprotection of the amino group of the compound (a-2) in the Preparation A-2.

2) Reaction of the Compound (a-4) with the Compound (d-3)

This reaction may be carried out in substantially the same manner as described above for the reaction of the compound (a-2) with the compound (d-1) in the Preparation A-2.

This Preparation is exemplified by Preparation 16 and the like.

Preparation A-5

The compound (a-6) can be prepared by deprotecting the carboxyl group of the compound (a-5).

The reaction may be carried out using a catalyst (e.g. Pearlman catalyst (Pd(OH)₂—C), palladium on carbon (Pd—C), and the like) under hydrogen atmosphere. The reaction may also be carried out using an alkali (e.g. sodium hydroxide, potassium hydroxide, lithium hydroxide, and the like).

The reaction may be carried out in a conventional solvent which does not adversely influence the reaction (e.g. methanol, ethanol, ethyl acetate, 1,4-dioxane, tetrahydrofuran, and the like).

The temperature of the reaction is not critical and the reaction is usually carried out from under cooling to heating.

This reaction is exemplified by Preparation 17 and the like.

Preparation A-6

The compound [II] may be prepared by deprotecting the amino group of the compound (a-6).

The reaction may be carried out in substantially the same manner as described for the deprotection of the amino group of the compound (a-2) in the Preparation A-2.

This Preparation is exemplified by Preparation 18 and the like.

Preparation A-5+6

Alternatively, when the carboxy protective group is t-butyl, the deprotection of carboxyl group and amino group of the compound (a-5) may be conducted simultaneously to give the Compound [II].

In this case, suitable deprotective agent for this reaction may be, for example, trifluoroacetic acid and the like.

The reaction may be carried out in a conventional solvent which does not adversely influence the reaction (e.g. dichloromethane, and the like).

The temperature of the reaction is not critical and the reaction is usually carried out from under cooling to heating.

This reaction is exemplified by Preparation 53, 57 and the like.

The compound [II] as obtained above is used in the Preparation C.

Preparation B

Preparation B-1

The compound (b-2) may be prepared by reacting the compound (b-1) with the compound (d-4).

The reaction may be carried out in the presence of a base (e.g. diisopropylethylamine) in suitable solvent (e.g. dichloromethane, ethyl acetate, 1,4-dioxane, methanol, ethanol, and the like).

The reaction may be carried out in a conventional solvent which does not adversely influence the reaction (e.g. dichloromethane and the like).

The temperature of the reaction is not critical and the reaction is usually carried out from under cooling to heating.

This Preparation is exemplified by Preparation 65 and the like.

Preparation B-2

The compound (b-3) may be prepared by 1) deprotecting the amino group of the compound (b-2), and 2) reacting the compound (b-2) with the compound (d-1).

1) Deprotection of the Amino Group of the Compound (b-2)

The reaction may be carried out in substantially the same manner as described above for the deprotection of the amino group of the compound (a-2) in the Preparation A-2.

2) Reaction of the Compound (b-2) with the Compound (d-1)

The reaction may be carried out in the presence of PyBOP®, HATU, and the like, and a base (e.g. Hünig base (e.g. N,N-diisopropylethylamine and the like) and the like).

The reaction may be carried out in a conventional solvent which, does not adversely influence the reaction (e.g. N,N-dimethylformamide and the like).

The temperature of the reaction is not critical and the reaction is usually carried out from under cooling to heating.

This Preparation is exemplified by Preparation 66 and the like.

Preparation B-3

The compound (b-4) may be prepared by 1) deprotecting the amino group of the compound (b-3), and 2) reacting the compound (b-3) with the compound (d-2).

1) Deprotection of the Amino Group of the Compound (b-3)

The reaction may be carried out in substantially the same manner as described above for the deprotection of the amino group of the compound (a-2) in the Preparation A-2.

2) Reaction of the Compound (b-3) with the Compound (d-2)

The reaction may be carried out in substantially the same manner as in Preparation B-2.

This Preparation is exemplified by Preparation 67 and the like.

Preparation B-4

The compound (b-5) may be prepared by 1) deprotecting the amino group of the compound (b-4), and 2) reacting the compound (b-4) with the compound (d-3).

1) Deprotection of the Amino Group of the Compound (b-4)

The reaction may be carried out in substantially the same manner as described above for the deprotection of the amino group of the compound (a-2) in the Preparation A-2.

2) Reaction of the Compound (b-4) with the Compound (d-3)

The reaction may be carried out in the presence of PyBOP®, HATU, and the like, and a base (e.g. Hünig base (e.g. N,N-diisopropylethylamine and the like) and the like).

The reaction may be carried out in a conventional solvent which does not adversely influence the reaction (e.g. dichloromethane, N,N-dimethylformamide, and the like).

The temperature of the reaction is not critical and the reaction is usually carried out from under cooling to heating.

This Preparation is exemplified by Preparation 68 and the like.

Preparation B-5

The compound [II] may be prepared by deprotecting the amino group and the carboxyl group attached to the resin unit of the compound (b-5).

The reaction may be carried out in the presence of an acid (e.g. trifluoroacetic acid and the like).

The reaction may be carried out in a conventional solvent which does not adversely influence the reaction (e.g. dichloromethane and the like).

The temperature of the reaction is not critical and the reaction is usually carried out from under cooling to heating.

This Preparation is exemplified by Preparation 69 and the like.

The compound [II] is used in the Preparation C.

Preparation C

Preparation C-1

The compound [III] may be prepared by cyclizing the compound [II].

The reaction may be carried out in the presence of a reagent (e.g. HATU, BOP, PyBOP®, TBTU, HOBT, and the like), and a base (e.g. dimethylaminopyridine, triethylamine, N,N-diisopropylethylamine, and the like).

The reaction may be carried out in a conventional solvent which does not adversely influence the reaction (e.g. N,N-dimethylformamide, methylene chloride, and the like).

The temperature of the reaction is not critical and the reaction is usually carried out from under cooling to heating.

This Preparation is exemplified by Preparation 76 and the like.

Preparation C-2

The compound [IV] may be prepared by deprotecting the hydroxyl group of the compound [III].

The reaction may be carried out in the presence of a base (e.g. sodium hydroxide, potassium hydroxide, lithium hydroxide, sodium methoxide, and the like).

The reaction may be carried out in a conventional solvent which does not adversely influence the reaction (e.g. methanol, ethanol, 1,4-dioxane, tetrahydrofuran, and the like).

The temperature of the reaction is not critical and the reaction is usually carried out from under cooling to heating.

This Preparation is exemplified by Preparation 77 and the like.

Preparation C-3

The compound [V] may be prepared by oxidation of the compound [IV].

Suitable oxidizing agent in the reaction may be, for example, Dess-Martin periodinane (i.e. 1,1,1-triacetoxy-1,1-dihydro-1,2-benziodoxol-3(1H)-one), and the like.

The reaction may be carried out in a conventional solvent which does not adversely influence the reaction (e.g. dichloromethane, dimethylsulfoxide, and the like).

The temperature of the reaction is not critical and the reaction is usually carried out from under cooling to heating.

This Preparation is exemplified by Preparation 78 and the like.

The compound [V] is used in the Preparation of the compound [I] of the present invention.

Preparation of the Compound [I] of the Present Invention

Preparation of the Compound [I-1]

The compound [I-1] may be prepared by reacting the compound [V] with the compound (d-5).

Suitable compound (d-5) for the reaction may be, for example, dimethyl (3R)-tert-butyldimethylsilyloxy-2-oxobutylphosphonate, dimethyl (3S)-tert-butyldimethylsilyloxy-2-oxobutylphosphonate, dimethyl (3R)-tert-butyldimethylsilyloxy-2-oxoheptylphosphonate, dimethyl 3-fluoro-2-oxopropylphosphonate, and the like.

The reaction may be carried out in the presence of a base (e.g. barium hydroxide octahydrate, barium hydroxide monohydrate, sodium hydroxide, potassium tert-butoxide, cesium carbonate, and the like).

The reaction may be carried out in a conventional solvent which does not adversely influence the reaction (e.g. tetrahydrofuran, tetrahydrofuran-water mixture, N,N-dimethylformamide, dimethylsulfoxide, acetonitrile, ethanol, 2-propanol, and the like).

The temperature of the reaction is not critical and the reactions are usually carried out from under cooling to heating.

The reaction may also be carried out in the presence of an organic base (e.g. Hünig base, DBU, and the like) and a lithium salt (e.g. lithium chloride, lithium bromide, lithium iodide, and the like), in a suitable solvent (e.g. acetonitrile, dimethylformamide, and the like) [Horner-Wadsworth-Emmons reaction].

The temperature of the reaction is not critical and the reactions are usually carried out from under cooling to heating.

The Preparation of the compound [I-1] is exemplified by Example 1 and the like.

Preparation of the Compound [I-2]

The compound [I-2] may be prepared by hydrogenation of alkenylene of the compound [I-1′].

Suitable catalyst for the hydrogenation may be, for example, palladium-BaSO₄ (Pd—BaSO₄), palladium on carbon (Pd—C), Pd(OH)₂ on carbon, and the like.

The reaction may be carried out in a conventional solvent which does not adversely influence the reaction (e.g. methanol, ethyl acetate, ethanol, 1,4-dioxane, and the like).

The temperature of the reaction is not critical and the reaction is usually carried out from under cooling to heating.

The Preparation of the compound [I-2] is exemplified by Example 3 and the like.

Preparation of the Compound [I-3]

The compound [I-3] may be prepared by deprotecting the hydroxyl group of the compound [I-1] or [I-2].

Suitable agent for the reaction may be, for example, tetrabutylammonium fluoride, pyridinium poly(hydrogen fluoride), hydrogen fluoride, cesium fluoride, potassium fluoride, and the like.

The reaction may be carried out in a conventional solvent which does not adversely influence the reaction (e.g. tetrahydrofuran, N,N-dimethylformamide, pyridine, and the like). Optionally, the reaction may be carried out in the presence of a catalyst (e.g. Pearlman catalyst (Pd(OH)₂—C), palladium on carbon (Pd—C), and the like) under hydrogen atmosphere.

The temperature of the reaction is not critical and the reaction is usually carried out from under cooling to heating.

The Preparation of the compound [I-3] is exemplified by Example 6 and the like.

To determine absolute configuration of the hydroxyl group of the compound [I-3] and to estimate optical purity of the isomer of the compound [I-3], the compound [I-3] is reacted with a reagent such as (R)-(−)-α-methoxy-α-trifluoromethyl-α-phenylacetyl chloride, (S)-(+)-α-methoxy-α-trifluoromethyl-α-phenylacetyl chloride, and the like.

The reaction may be carried out in a conventional solvent which does not adversely influence the reaction (e.g. pyridine, methylene chloride, and the like).

The temperature of the reaction is not critical and the reaction is usually carried out from under cooling to heating.

This reaction is exemplified by Example 53.

Preparation of the Compound [I-4]

The compound [I-4] may be prepared by reacting the compound [I-3′] with sodium periodate.

The reaction may be carried out in a conventional solvent which does not adversely influence the reaction (e.g. water, methanol, and the like).

The temperature of the reaction is not critical and the reaction is usually carried out from under cooling to heating.

The Preparation of the compound [I-4] is exemplified by Example 139 and the like.

Preparation of the compound [I-5]

The compound [I-5] may be prepared by reacting the compound [I-4] with the compound (d-6).

Suitable agent for the reaction may be, for example, carbodiimide [e.g. 1-ethyl-3-(3′-N,N-dimethylaminopropyl)-carbodiimide (EDC) or hydrochrolide thereof, dicyclohexylcarbodiimide (DCC), and the like], benzotriazol-1-yloxy-tris-pyrrolidinophosphonium hexafluorophosphate (PyBOP®), benzotriazol-1-yloxy-tris-(dimethylamino)phosphonium hexafluoro phosphate (BOP), bromo-tris-pyrrolidinophosphonium hexafluorophosphate (PyBroP®), 1,1′-carbonyldiimidazol (CDI), diphenylphosphoryl azide (DPPA), 1-hydroxybenzotriazole (HOBT), benzotriazol-1-yloxy-tris-(dimethylamino)phosphonium-hexafluorophosphate (HATU), 2-(1H-benzotriazol-1-yl)-1,1,3,3-tetramethyluromium tetrafluoroborate (TBTU), 2-(1H-benzotriazole-1-yl)-1,1,3,3-tetramethyluronium hexafluorophosphate (HBTU) and the like.

The reaction may be carried out in a conventional solvent which does not adversely influence the reaction (e.g. dichloromethane, N,N-dimethylformamide and the like).

The temperature of the reaction is not critical and the reaction is usually carried out from under cooling to heating.

The Preparation of the compound [I-5] is exemplified by Example 141 and the like.

Preparation of the Compound [I-6]

The compound [I-6] may be prepared by reacting the compound [I-5] with Grignard's agent [e.g. alkylmagnesium halide (R¹¹MgQ)].

Suitable alkylmagnesium halide for the reaction may be, for example, methyl magnesium bromide, ethyl magnesium bromide, phenyl magnesium bromide, benzyl magnesium bromide and the like.

The reaction may be carried out in a conventional solvent which does not adversely influence the reaction (e.g. tetrahydrofuran, diethylether and the like).

The temperature of the reaction is, for example, −78° C. to 0° C.

The Preparation of the compound [I-6] is exemplified by Example 143 and the like.

Preparation of the Compound [I-7]

The compound [I-7] may be prepared by reducing the compound [I-1] with a reductant.

Suitable reductant for the reaction may be, for example, sodium borohydride, lithium aluminum hydride, diisobutylalminum hydride, sodium cyanoborohydride, sodium triacetoxyborohydride and the like.

The reaction may be carried out in a conventional solvent which does not adversely influence the reaction (e.g. methanol, ethanol, tetrahydrofuran, dioxane, 2-propanol and the like).

The temperature of the reaction is not critical and the reaction is usually carried out from under cooling to heating.

The Preparation of the compound [I-7] is exemplified by Example 147 and the like.

Preparation of the Compound [I-8]

The compound [I-8] may be prepared by fluoridation of a hydroxyl group of the compound [I-3′] with diethylaminosulfurtrifluoride.

The reaction may be carried out in a conventional solvent which does not adversely influence the reaction (e.g. dichloromethane, acetonitrile, acetic acid, chloroform, tetrahydrofuran, 2-propanol and the like).

The temperature of the reaction is not critical and the reaction is usually carried out from under cooling to heating.

The Preparation of the compound [I-8] is exemplified by Example 148 and the like.

Preparation of the Compound [I-9]

The compound [I-9] may be prepared by reacting the compound. [I-5] with alkyllithium (R¹²Li).

Suitable alkyllithium for the reaction may be, for example, n-butyllithium, methyllithium ethyllithium, isopropyllithium, isobutyllithium, tert-butyllithium, n-hexyllithium, phenyllithium, vinyllithium and the like.

The reaction may be carried out in a conventional solvent which does not adversely influence the reaction (e.g. tetrahydrofuran, diethyl ether, cyclohexane and the like).

The temperature of the reaction is, for example, −78° C. to 0° C.

The Preparation of the compound [I-9] is exemplified by Example 149 and the like.

Preparation of the Compound [I-10]

The compound [I-10] may be prepared by reacting the compound [I-1″] with a secondary amine (R¹³R¹⁴NH).

Suitable secondary amine for this reaction may be, for example, piperidine, morpholine, dicyclohexylamine, diethylamine and the like.

The reaction may be carried out in a conventional solvent which does not adversely influence the reaction (e.g. N,N-dimethylformamide, and the like).

The temperature of the reaction is not critical and the reaction is usually carried out from under cooling to heating.

Preparation of the Compound [I-11]

The compound [I-11] may be prepared by reacting the compound [I-10] with methanesulfonyl chloride.

The reaction may be carried out in a conventional solvent which does not adversely influence the reaction (e.g. pyridine, dichloromethane, and the like).

The temperature of the reaction is, for example, 0° C. to room temperature.

Preparation of the Compound [I-12]

The compound [I-12] may be prepared by reacting the compound [I-10] with acetic anhydride in the presence of a catalytic amount of 4-(dimethylamino)pyridine.

The reaction may be carried out in a conventional solvent which does not adversely influence the reaction (e.g. pyridine, dichloromethane and the like).

The temperature of the reaction is not critical and the reaction is usually carried out from under cooling to heating.

Preparation of the Compound [I-13]

The compound [I-13] may be prepared by reacting the compound [I-3″] with sodium periodate under the catalytic amount of rubidium oxide.

The reaction may be carried out in a conventional solvent which does not adversely influence the reaction (e.g., a mixed solvent of carbon tetrachloride acetonitrile and water, and the like).

The temperature of the reaction is not critical and the reaction is usually carried out from under cooling to heating.

The Preparation of the compound [I-13] is exemplified by Example 163 and the like.

Preparation of the Compound [I-14]

The compound [I-14] may be prepared by reacting the compound. [I-13] with a primary amine (R¹⁵—NH₂).

The reaction may be carried out in the presence of PyBOP®, HATU, and the like, and a base (e.g. Hünig base (e.g. N,N-diisopropylethylamine and the like) and the like).

The reaction may be carried out in a conventional solvent which does not adversely influence the reaction (e.g. N,N-dimethylformamide and the like).

The temperature of the reaction is not critical and the reaction is usually carried out from under cooling to heating.

The Preparation of the compound [I-14] is exemplified by Example 164 and the like.

Preparation of the Compound [I-15]

The compound [I-15] may be prepared by reacting the compound [I-3′″] with a primary amine (R¹⁶—NH₂).

The reaction may be carried out in a conventional solvent which does not adversely influence the reaction (e.g. methanol and the like).

The temperature of the reaction is not critical and the reaction is usually carried out from under cooling to heating.

The Preparation of the compound [I-15] is exemplified by Example 253 and the like.

Test Method

In order to show the usefulness of the compound [I] of the invention, the pharmacological test result of the representative compounds of the present invention is shown in the following.

Test 1: Determination of Histone Deacetylase Inhibitor Activity

The partial purification of human histone deacetylase, the preparation of [³H] acetyl histones, and the assay for histone deacetylase activity were performed basically according to the method as proposed by Yoshida et al. as follows.

Partial Purification of Human Histone Deacetylase

The human histone deacetylase was partially purified from human T cell leukemia Jurkat cells. Jurkat cells (5×10⁸ cells) were suspended in 40 ml of the HDA buffer consisting of 15 mM potassium phosphate, pH 7.5, 5% glycerol and 0.2 mM EDTA. After homogenization, nuclei were collected by centrifugation (35,000×g, 10 min) and homogenized in 20 ml of the same buffer supplemented with 1 M (NH₄)₂SO₄. The viscous homogenate was sonicated and clarified by centrifugation (35,000×g, 10 min), and the deacetylase was precipitated by raising the concentration of (NH₄)₂SO₄ to 3.5 M. The precipitated protein was dissolved in 10 ml of the HDA buffer and dialyzed against 4 liters of the same buffer. The dialyzate was then loaded onto a DEAE-cellulose (Whatman DE52) column (25×85 mm) equilibrated with the same buffer and eluted with 300 ml of a linear gradient (0-0.6 M) of NaCl. A single peak of histone deacetylase activity appeared between 0.3 and 0.4 M NaCl.

Preparation of [³H] Acetyl Histone

To obtain [³H] acetyl-labeled histone as the substrate for the histone deacetylase assay, 1×10⁸ cells of Jurkat in 20 ml of RPMI-1640 medium (supplemented with 10% FBS, penicillin (50 units/ml) and streptomycin (50 μg/ml)) were incubated with 300 MBq [³H] sodium acetate in the presence of 5 mM sodium butyrate for 30 minutes in 5% CO₂-95% air atmosphere at 37° C. in a 75 cm² flask, harvested into a centrifuge tube (50 ml), collected by centrifugation at 1000 rpm for 10 minutes, and washed once with phosphate-buffered saline. The washed cells were suspended in 15 ml of ice-cold lysis buffer (10 mM Tris-HCl, 50 mM sodium bisulfite, 1% Triton X-100, 10 mM MgCl₂, 8.6% sucrose, pH 6.5). After Dounce homogenization (30 stroke), the nuclei were collected by centrifugation at 1000 rpm for 10 minutes, washed 3 times with 15 ml of the lysis buffer, and once with 15 ml of ice-cooled washing buffer (10 mM Tris-HCl, 13 mM EDTA, pH 7.4) successively. The pellet was suspended in 6 ml of ice-cooled water using a mixer, and 68 μl of H₂SO₄ was added to the suspension to give a concentration of 0.4 N. After incubation at 4° C. for 1 hour, the suspension was centrifuged for 5 minutes at 15,000 rpm, and the supernatant was taken and mixed with 60 ml of acetone. After overnight incubation at −20° C., the coagulated material was collected by microcentrifugation, air-dried, and stored at −80° C.

Assay for Histone Deacetylase Activity

For the standard assay, 10 μl of [³H] acetyl-labeled histones were added to 90 μl of the enzyme fraction, and the mixture was incubated at 25° C. for 30 minutes. The reaction was stopped by addition of 10 μl of HCl. The released [³H] acetic acid was extracted with 1 ml of ethyl acetate, and 0.9 ml of the solvent layer was taken into 10 ml of toluene scintillation solution for determination of radioactivity.

Test 2: Determination of T-Cell Growth Inhibitor Activity

The T lymphocyte blastogenesis test was performed in microtiter plates with each well containing 1.5×10⁵ splenic cells of Lewis rats in 0.1 ml RPMI-1640 medium supplemented with 10% fetal bovine serum (FBS), 50 mM 2-mercaptoethanol, penicilln (100 units/ml) and streptomycin (100 μg/ml), to which Concanavalin A (1 μg/ml) was added. The cells were incubated at 37° C. in a humidified atmosphere of 5% CO₂ for 72 hours. After the culture period, suppressive activities of the test compounds in T lymphocyte blastogenesis were quantified by AlamarBlue (trademark) Assay. The test samples were dissolved in DMSO and further diluted with RPMI-1640 medium and added to the culture. The activities of the test compounds were expressed as IC₅₀.

The results of those tests are shown in the Table 1.

TABLE 1
HDAC inhibitory activity and T-cell growth inhibitory
activity of the compound of the present invention

| | | Test 2: T-cell |
|---|---|---|
| | | growth |
| | Test 1: HDAC | inhibitory |
| | inhibitory | activity IC₅₀ |
| Examples | activity IC₅₀ (nM) | (nM) |
| Compound E6 | <100 | <50 |
| Compound E8 | <100 | <50 |
| Compound E13 | <100 | <50 |
| Compound E17 | <100 | <50 |
| Compound E23 | <100 | <50 |
| Compound E26 | <100 | <50 |
| Compound E33 | <100 | <50 |
| Compound E35 | <100 | <50 |
| Compound E38 | <100 | <50 |
| Compound E41 | <100 | <50 |
| Compound E48 | <100 | <50 |

Test 3: Effect of HDAC Inhibitor on TNFα Induced NF-κB Activation

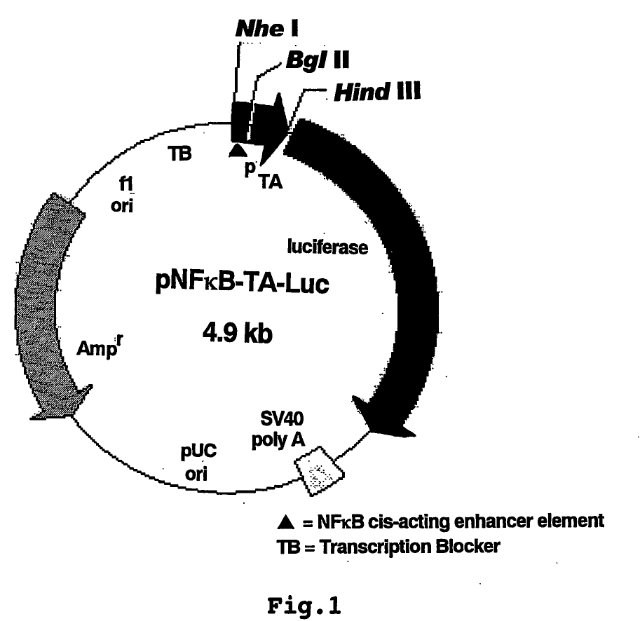

8.75×10⁶ HEL cells (JCRB0062, JCRB) were transfected with 10 μg of pNFκB-TA-Luc (Clontech, as shown in FIG. 1) by electroporation at 1750 V and 10 μF with Gene Pulser II (BIO-RAD). The cells were resuspended in 2 ml of RPMI1640 (SIGMA) supplemented with 10% FBS (MOREGATE) and aliquated 50 μl each well in 96-well tissue culture plates. After 5 hr culture at 37° C., 5% CO₂, for cell function recovery, the cells were cultured in the presence or absence of TNFα (10 ng/ml) for 4 hr. TNFα stimulated cells were incubated with Compound E138 or FK506 (commercial available immunosuppresive agent, also referred as Tacrolimus) at appropriate concentrations for 1 hour prior to stimulation.

For the NF-κB reporter gene assay, the transfected cells were lysed and assayed for luciferase activity with the Bright-glo Luciferase Assay System (Promega) according to the manufacturer's instructions.

For the cell growth analysis, the transfected cells were analyzed using Cell Counting Kit8 (Dojin) according to the manufacturer's instructions.

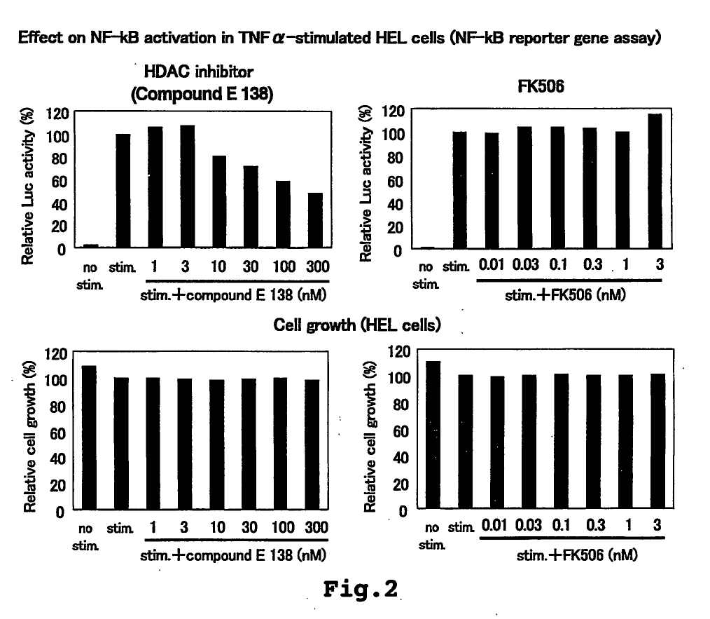

Results of the study are shown in FIG. 2. Treatment of the transfected cells with TNFα induces NF-κB-dependent luciferase expression. Compound E138 has an inhibitory effect on TNFα induced NF-κB activation in a dose-dependent manner without affecting cell growth. In contrast to the effect of Compound E138, FK506 has no direct effect on TNFα induced NF-κB activation at doses up to 3 nM (FK506 almost completely inhibits IL-2 mRNA expression in activated Jurkat cells at 1 nM). Therefore, HDAC inhibitor (Compound E138) has an inhibitory effect on NF-κB activation induced by TNFα, a calcium-signaling-independent NF-κB activation, whereas FK506 has no direct effect on it.

Test 4: Effect of HDAC Inhibitor on MCP-1 Production by Activated THP-1 Cells

For the measurement of MCP-1 level by ELISA, 1×10⁶ THP-1 cells (JCRB0112, JCRB) were plated in 6-well tissue culture plates. The cells were cultured in RPMI1640 (SIGMA) supplemented with 10% FBS (MOREGATE) in the presence of PMA (SIGMA, 50 ng/mL) for 16 hours at 37° C., 5% CO₂. After incubation, the medium was changed to RPMI1640 supplemented with 2% FBS and various concentrations of Compound E138 or FK506 were added. The cells were further cultured for 9 hr and the amount of MCP-1 protein secreted by activated THP-1 cells into the medium was determined by ANALYZA Immuno assay System human MCP-1 (Genzyme Techne) according to the manufacturer's instructions.

For the cell growth analysis, 5×10⁴ THP-1 cells were plated in 96-well tissue culture plates. The cells were cultured in RPMI1640 supplemented with 10% FBS in the presence of PMA (50 ng/mL) for 16 hours at 37° C., 5% CO₂. After incubation, the medium was changed to RPMI1640 supplemented with 2% FBS and various concentrations of Compound E138 or FK506 were added. The cells were further cultured for 9 hr and analyzed using Cell Counting Kit8 (Dojin) according to the manufacturer's instructions.

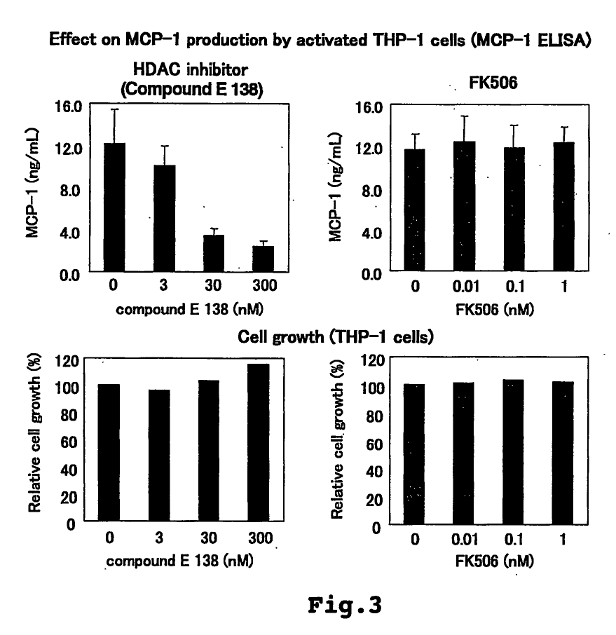

Results of the study are shown in FIG. 3. Treatment of the cells with PMA induces MCP-1 expression. Compound E138 has an inhibitory effect on MCP-1 production by activated THP-1 cells in a dose-dependent manner without affecting cell growth. In contrast to the effect of Compound E138, FK506 has no direct effect on MCP-1 production by activated THP-1 cells at doses up to 1 nM (FK506 almost completely inhibits IL-2 mRNA expression in activated Jurkat cells at 1 nM).

These results demonstrate that HDAC inhibitors such as Compound E138 is a new class of the immunosuppressive agents that inhibit MCP-1-dependent chronic inflammation.

The pharmaceutical composition of the present invention comprising histone deacetylase inhibitor, such as the compound [I], is useful as a therapeutic or prophylactic agent for diseases caused by abnormal gene expression, such as inflammatory disorders, diabetes, diabetic complications, homozygous thalassemia, fibrosis, cirrhosis, acute promyelocytic leukaemia (APL), protozoal infection and the like. Further, it is useful as an antitumor agent or immunosuppressant, which prevents an organ transplant rejection and autoimmune diseases as exemplified below.

Rejection reactions by transplantation of organs or tissues such as the heart, kidney, liver, bone marrow, skin, cornea, lung, pancreas, small intestine, limb, muscle, nerve, intervertebral disc, trachea, myoblast, cartilage, and the like;

graft-versus-host reactions following bone marrow transplantation; autoimmune diseases such as rheumatoid arthritis, systemic lupus erythematosus, Hashimoto's thyroiditis, multiple sclerosis, myasthenia gravis, type I diabetes, and the like;

and infections caused by pathogenic microorganisms (e.g. Aspergillus fumigatus, Fusarium oxysporum, Trichophyton asteroides, and the like).

Furthermore, pharmaceutical preparations of the histone deacetylase inhibitor, such as the compound [I], are useful for the therapy or prophylaxis of the following diseases.

Inflammatory or hyperproliferative skin diseases or cutaneous manifestations of immunologically-mediated diseases (e.g. psoriasis, atopic dermatitis, contact dermatitis, eczematoid dermatitis, seborrheic dermatitis, lichen planus, pemphigus, bullous pemphigoid, epidermolysis bullosa, urticaria, angioedema, vasculitides, erythema, dermal eosinophilia, lupus erythematosus, acne, and alopecia areata);

autoimmune diseases of the eye (e.g. keratoconjunctivitis, vernal conjunctivitis, uveitis associated with Behcet's disease, keratitis, herpetic keratitis, conical keratitis, corneal epithelial dystrophy, keratoleukoma, ocular premphigus, Mooren's ulcer, scleritis, Grave's ophthalmopathy, Vogt-Koyanagi-Harada syndrome, keratoconjunctivitis sicca (dry eye), phlyctenule, iridocyclitis, sarcoidosis, endocrine ophthalmopathy, and the like);

reversible obstructive airways diseases [asthma (e.g. bronchial asthma, allergic asthma, intrinsic asthma, extrinsic asthma, and dust asthma), particularly chronic or inveterate asthma (e.g. late asthma and airway hyper-responsiveness) bronchitis, and the like];

mucosal or vascular inflammations (e.g. gastric ulcer, ischemic or thrombotic vascular injury, ischemic bowel diseases, enteritis, necrotizing enterocolitis, intestinal damages associated with thermal burns, leukotriene B4-mediated diseases);

intestinal inflammations/allergies (e.g. coeliac diseases, proctitis, eosinophilic gastroenteritis, mastocytosis, Crohn's disease and ulcerative colitis);

food-related allergic diseases with symptomatic manifestation remote from the gastrointestinal tract (e.g. migrain, rhinitis and eczema);

renal diseases (e.g. intestitial nephritis, Goodpasture's syndrome, hemolytic uremic syndrome, and diabetic nephropathy);

nervous diseases (e.g. multiple myositis, Guillain-Barre syndrome, Meniere's disease, multiple neuritis, solitary neuritis, cerebral infarction, Alzheimer's disease, Parkinson's disease, amyotrophic lateral sclerosis (ALS), and radiculopathy);

cerebral ischemic diseases (e.g., head injury, hemorrhage in brain (e.g., subarachnoid hemorrhage, intracerebral hemorrhage), cerebral thrombosis, cerebral embolism, cardiac arrest, stroke, transient ischemic attack (TIA), and hypertensive encephalopathy);

endocrine diseases (e.g. hyperthyroidism, and Basedow's disease);

hematic diseases (e.g. pure red cell aplasia, aplastic anemia, hypoplastic anemia, idiopathic thrombocytopenic purpura, autoimmune hemolytic anemia, agranulocytosis, pernicious anemia, megaloblastic anemia, and anerythroplasia);

bone diseases (e.g. osteoporosis);

respiratory diseases (e.g. sarcoidosis, pulmonary fibrosis, and idiopathic interstitial pneumonia);

skin diseases (e.g. dermatomyositis, leukoderma vulgaris, ichthyosis vulgaris, photosensitivity, and cutaneous T-cell lymphoma);

circulatory diseases (e.g. arteriosclerosis, atherosclerosis, aortitis syndrome, polyarteritis nodosa, and myocardosis);

collagen diseases (e.g. scleroderma, Wegener's granuloma, and Sjögren's syndrome);

adiposis;

eosinophilic fasciitis;

periodontal diseases (e.g. damage to gingiva, periodontium, alveolar bone or substantia ossea dentis);

nephrotic syndrome (e.g. glomerulonephritis);

male pattern alopecia, alopecia senile;

muscular dystrophy;

pyoderma and Sezary syndrome;

chromosome abnormality-associated diseases (e.g. Down's syndrome);

Addison's disease;

active oxygen-mediated diseases [e.g. organ injury (e.g. ischemic circulation disorders of organs (e.g. heart, liver, kidney, digestive tract, and the like) associated with preservation, transplantation, or ischemic diseases (e.g. thrombosis, cardial infarction, and the like):

intestinal diseases (e.g. endotoxin shock, pseudomembranous colitis, and drug- or radiation-induced colitis);

renal diseases (e.g. ischemic acute renal insufficiency, chronic renal failure);

pulmonary diseases (e.g. toxicosis caused by pulmonary oxygen or drugs (e.g. paracort, bleomycin, and the like), lung cancer, and pulmonary emphysema);

ocular diseases (e.g. cataracta, iron-storage disease (siderosis bulbi), retinitis, pigmentosa, senile plaques, vitreous scarring, corneal alkali burn);

dermatitis (e.g. erythema multiforme, linear immunoglobulin A bullous dermatitis, cement dermatitis);

and other diseases (e.g. gingivitis, periodontitis, sepsis, pancreatitis, and diseases caused by environmental pollution (e.g. air pollution), aging, carcinogen, metastasis of carcinoma, and hypobaropathy)];

diseases caused by histamine release or leukotriene C4 release; restenosis of coronary artery following angioplasty and prevention of postsurgical adhesions;

autoimmune diseases and inflammatory conditions (e.g., primary mucosal edema, autoimmune atrophic gastritis, premature menopause, male sterility, juvenile diabetes mellitus, pemphigus vulgaris, pemphigoid, sympathetic ophthalmitis, lens-induced uveitis, idiopathic leukopenia, active chronic hepatitis, idiopathic cirrhosis, discoid lupus erythematosus, autoimmune orchitis, arthritis (e.g. arthritis deformans), or polychondritis);

Human Immunodeficiency Virus (HIV) infection, AIDS;

allergic conjunctivitis;

hypertrophic cicatrix and keloid due to trauma, burn, or surgery.

Therefore, the pharmaceutical composition of the present invention is useful for the therapy and prophylaxis of liver diseases [e.g. immunogenic diseases (e.g. chronic autoimmune liver diseases such as autoimmune hepatic diseases, primary biliary cirrhosis or sclerosing cholangitis), partial liver resection, acute liver necrosis (e.g. necrosis caused by toxins, viral hepatitis, shock, or anoxia), hepatitis B, non-A non-B hepatitis, hepatocirrhosis, and hepatic failure (e.g. fulminant hepatitis, late-onset hepatitis and “acute-on-chronic” liver failure (acute liver failure on chronic liver diseases))].

The pharmaceutical composition of the present invention can be used in the form of pharmaceutical preparation, for example, in a solid, semisolid or liquid form, which contains the histone deacetylase inhibitor, such as the compound [I], as an active ingredient in admixture with an organic or inorganic carrier or excipient suitable for external, enteral or parenteral administrations. The active ingredient may be compounded, for example, with the usual non-toxic, pharmaceutically acceptable carriers for tablets, pellets, capsules, suppositories, solutions, emulsions, suspensions, injections, ointments, liniments, eye drops, lotion, gel, cream, and any other form suitable for use.

The carriers which can be used are water, glucose, lactose, gum acacia, gelatin, mannitol, starch paste, magnesium trisilicate, talc, corn starch, keratin, colloidal silica, potato starch, urea and other carriers suitable for use in manufacturing preparations, in a solid, semisolid, or liquid form, and in addition auxiliary, stabilizing, thickening, solubilizing and coloring agents and perfumes may be used.

For applying the composition to human, it is preferable to apply it by intravenous, intramuscular, topical or oral administration, or by a vascular stent impregnated with the compound [I]. While the dosage of therapeutically effective amount of the histone deacetylase inhibitor, such as the compound [I], varies from and also depends upon the age and condition of each individual patient to be treated, when an individual patient is to be treated, in the case of intravenous administration, a daily dose of 0.01-10 mg of the histone deacetylase inhibitor, such as the compound [I], per kg weight of human being, in the case of intramuscular administration, a daily dose of 0.1-10 mg of the histone deacetylase inhibitor, such as the compound of the formula [I], per kg weight of human being, and in the case of oral administration, a daily dose of 0.5-50 mg of the histone deacetylase inhibitor, such as the compound [I], per kg weight of human being, is generally given for treatment.

During the preparation of the above-mentioned pharmaceutical administration forms, the compound [I] or a salt thereof can also be combined together with other immunosuppressive substances, for example repamycin, mycophenolic acid, cyclosporin A, tacrolimus or brequinar sodium.

The following Preparations and Examples are given for the purpose of illustrating the present invention in more detail.

Preparation 1

To a stirred solution of 2(S)-(+)-amino-2-methylbutanoic acid monohydrate (15 g) in 1,4-dioxane (225 ml), a mixture of 1N sodium hydroxide aqueous solution (111 ml) and di-tert-butyl dicarbonate (24.2 g) was added at ambient temperature and the resulting mixture was stirred for 53 hours. Additional mixture of di-tert-butyl dicarbonate (24.2 g) and 1N sodium hydroxide aqueous solution (111 ml) was added at 8 hours, 24 hours and 48 hours after the start of the reaction. The mixture was diluted with diethyl ether (400 ml) and the organic layer was separated. The pH of the aqueous phase was adjusted to 1 with concentrated hydrochloric acid. The aqueous phase was extracted with ethyl acetate (500 ml) twice and the organic layers were combined, washed with brine (500 ml), dried over anhydrous sodium sulfate and concentrated in vacuo. The residual solid was treated with hexane (100 ml) and the resulting suspension was stirred in an ice bath for one hour. The precipitate was filtered and washed with cold hexane to afford 2(S)-N-tert-butoxycarbonylamino-2-methylbutanoic acid (21.71 g, hereinafter Compound (1)) as a white amorphous solid.

¹H-NMR (300 MHz, DMSO-d₆, δ): 6.82 (1H, brs), 1.61-1.82 (2H, m), 1.36 (9H, s), 0.75 (3H, t, J=7.5 Hz);

MASS (ES−): m/e 216.17.

Preparation 2

To a solution of (S)-2-amino-6-hydroxyhexanoic acid (2.0 g) and sodium bicarbonate (2.28 g) in dioxane-water mixture (20 ml:20 ml) was added di-tert-butyl dicarbonate (5.93 g) at room temperature. The resulting mixture was stirred at room temperature for 6 hours. The reaction mixture was diluted with water and washed with ether. The aqueous phase was adjusted to pH 2 with conc. hydrochloric acid and extracted with ethyl acetate. The organic layer was washed with water and brine, dried over anhydrous sodium sulfate and concentrated in vacuo to give 2(S)-N-tert-butoxycarbonylamino-6-hydroxyhexanoic acid as a solid.

¹H-NMR (300 MHz, DMSO-d₆, δ): 1.18-1.45 (4H, m), 1.37 (9H, s), 1.45-1.70 (2H, m), 3.35 (2H, m), 3.75-3.88 (1H, m), 4.31-4.45 (1H, br), 7.06 (1H, d, J=7.5 Hz);

MASS (ES−): m/e 246.15 (M−1).

Preparation 3

To a solution of 2(S)-N-tert butoxycarbonylamino-6-hydroxyhexanoic acid (3.36 g) in N,N-dimethylformamide (35 ml), cesium carbonate powder was added (2.21 g) at 0° C. and stirred for 1.5 hours at room temperature. To the mixture, benzylbromide (1.66 ml) was added at 0° C. and stirred for 1.5 hours. The reaction mixture was stirred for further 1.5 hours at room temperature. The reaction mixture was poured into water under ice-cooling and extracted with ethyl acetate. The organic layer was washed with water (3 times) and brine, dried over anhydrous sodium sulfate and concentrated in vacuo to give 2(S)-N-tert-butoxycarbonylamino-6-hydroxyhexanoic acid benzyl ester as a pale yellow crude oil.

¹H-NMR (300 MHz, CDCl₃, δ): 1.44 (9H, s), 1.48-1.90 (7H, m), 3.55-3.65 (2H, m), 4.30-4.41 (1H, m), 5.02-5.10 (1H, m), 5.10-5.25 (2H, m), 7.36 (5H, brs);

MASS (ES−): m/e 338.23 (M+1).

Preparation 4

To a solution of 2(S)-N-tert-butoxycarbonylamino-6-hydroxyhexanoic acid benzyl ester (4.58 g) in pyridine (13 ml), benzoylchloride (2 g) was added at 0° C. and stirred for 1.5 hours at room temperature. The reaction mixture was poured into cooled 1N hydrochloric acid (150 ml) and stirred for 10 minutes. The resulting mixture was extracted with ethyl acetate. The organic layer was washed with water, saturated aqueous sodium bicarbonate solution, water and brine, dried over anhydrous sodium sulfate and concentrated in vacuo. The crude product was purified by column chromatography (eluting with ethyl acetate/hexane=10/1 to 4/1 v/v) to give 2(S)-N-tert-butoxycarbonylamino-6-benzoyloxyhexanoic acid benzyl ester as a pale yellow oil.

¹H-NMR (300 MHz, CDCl₃, δ): 1.35-1.60 (2H, m), 1.43 (9H, s), 1.62-1.96 (4H, m), 4.26 (1H, t, J=6.0 Hz), 4.30-4.42 (1H, m), 5.00-5.08 (1H, m), 5.08-5.22 (2H, m), 7.34 (5H, s), 7.39-7.46 (2H, m), 7.52-7.60 (1H, m), 7.98-8.05 (2H, m);

MASS (ES+): m/e 442.34.

Preparation 5

To a solution of 2(S)-N-tert-butoxycarbonylamino-6-benzoyloxyhexanoic acid benzyl ester (5.43 g) in methanol (55 ml), palladium hydroxide on charcoal catalyst (50 mg) was added. The air atmosphere was replaced with hydrogen (4 atm) and shaken for 3 hours. The resulting mixture was filtered through a pad of Celite®, and washed with methanol. The filtrate was concentrated in vacuo to give 6-benzoyloxy-2(S)-N-tert-butoxycarbonylaminohexanoic acid (hereinafter Compound (5)) as a pale yellow oil.

¹H-NMR (300 MHz, CDCl₃, δ): 1.44 (9H, s), 1.47-2.05 (6H, m), 4.12-4.27 (1H, m), 4.44 (2H, t, J=6.0 Hz), 5.00-5.12 (1H, m), 7.38-7.50 (2H, m), 7.50-7.62 (1H, m), 8.00-8.07 (2H, m);

MASS (ES+): m/e 352.20 (M+1).

Preparation 6

To a cooled suspension of N-tert-butoxycarbonylamino-6-methoxy-6-oxo-L-norleucine dicyclohexylamine salt (21.1 g) in N,N-dimethylformamide (210 ml) was added benzyl bromide (7.9 g), and the mixture was stirred at ambient temperature for 3 days. The mixture was evaporated in vacuo. The residue was diluted with ethyl acetate and the remaining solid was filtered off. The filtrate was washed with 10% aqueous citric acid solution, saturated aqueous sodium bicarbonate solution and brine, dried over magnesium sulfate and evaporated in vacuo. The residue was purified by silica gel column chromatography (eluting with hexane/ethyl acetate=4:1 to 2:1 v/v) to give N-tert-butoxycarbonyl-6-methoxy-6-oxo-L-norleucine benzyl ester (15.4 g) as a white solid.

¹H-NMR (300 MHz, CDCl₃, δ): 1.28 (3×3H, s), 1.59-1.75 (3H, m), 1.83 (1H, m), 2.31 (2H, m), 3.65 (3H, s), 4.35 (1H, m), 5.06 (1H, brd, J=8 Hz), 5.14 (1H, d, J=12 Hz), 5.20 (1H, d, J=12 Hz), 7.30-7.42 (5H, m);

MASS (ES+): m/e 366.

Preparation 7

To a stirred solution of N-tert-butoxycarbonyl-6-methoxy-6-oxo-L-norleucine benzyl ester (15.4 g) in acetonitrile (150 ml) were added 4-dimethylaminopyridine (1.03 g) and di-tert-butyldicarbonate (14.7 g), and the mixture was stirred at ambient temperature for 1 day. The mixture was evaporated in vacuo. The residue was purified by silica gel column chromatography (eluting with hexane/ethyl acetate=10:1 v/v) to give N,N-di-tert-butoxycarbonyl-6-methoxy-6-oxo-L-norleucine as a colorless oil (20.0 g).

¹H-NMR (300 MHz, CDCl₃, δ): 1.45 (2×9H, s), 1.70 (2H, m), 1.96 (1H, m), 2.15 (1H, m), 2.36 (2H, m), 3.66 (3H, s), 4.90 (1H, dd, J=9, 4.5 Hz), 5.13 (1H, d, J=11 Hz), 5.17 (1H, d, J=11 Hz), 7.28-7.39 (5H, m);

MASS (ES+): m/e 488.

Preparation 8

To a cooled solution of N,N-di-tert-butoxycarbonyl-6-methoxy-6-oxo-L-norleucine (9.71 g) in diethyl ether (150 ml) was added dropwise 1M solution of diisobutylaluminium hydride in hexane (DIBAL) (23 ml) at −78° C. After 30 minutes DIBAL (24 ml) was added dropwise until the starting compound was disappeared. The reaction mixture was quenched by addition of water. After warming to 0° C. with stirring, the mixture was filtered through a pad of Celite®. The solvent was evaporated and the residual solvent was removed azeotropically with toluene to give N,N-di-tert-butoxycarbonyl-6-oxo-L-norleucine benzyl ester as a pale yellow oil (8.94 g).

¹H-NMR (300 MHz, CDCl₃, δ): 1.45 (2×9H, s), 1.70 (2H, m), 1.96 (1H, m), 2.14 (1H, m), 2.49 (2H, m), 4.90 (1H, m), 5.13 (1H, d, J=12 Hz), 5.17 (1H, d, J=12 Hz), 7.26-7.39 (5H, m), 9.76 (1H, t, J=1 Hz);

MASS (ES−): m/e 435.

Preparation 9

To a stirred solution of dimethyl (3R)-3-benzyloxy-2-oxobutylphosphonate (1.08 g), lithium chloride (174 mg), and N,N-diisopropylethylamine (442 mg) in acetonitrile (10 ml) was added a solution of N,N-di-tert-butoxycarbonyl-6-oxo-L-norleucine benzyl ester (1.49 g) in acetonitrile (30 ml) at ambient temperature. The mixture was stirred at ambient temperature for 5 days. After evaporation of the solvent, the residue was diluted with water, and extracted with ethyl acetate. The extract was washed with brine, dried over magnesium sulfate, and the solvent was evaporated in vacuo. The residue was purified by silica gel column chromatography (eluting with hexane/ethyl acetate=10:1 v/v) to give benzyl (2S,6E)-9-benzyloxy-2-di-tert-butoxycarbonylamino-8-oxodec-6-enoate as an oil (1.13 g).

¹H-NMR (300 MHz, CDCl₃, δ): 1.35 (3H, d, J=7 Hz), 1.38-1.62 (6H, m), 1.44 (2×9H, s), 1.95 (1H, m), 2.16 (1H, m), 2.28 (2H, m), 4.05 (1H, q, J=7 Hz), 4.41 (1H, d, J=12 Hz), 4.56 (1H, d, J=12 Hz), 4.90 (1H, dd, J=10 and 5 Hz), 5.12 (1H, d, J=12 Hz), 5.16 (1H, d, J=12 Hz), 6.51 (1H, d, J=15 Hz), 7.02 (1H, dt, J=15, 7 Hz), 7.23-7.40 (5H, m);

MASS (ES+): m/e 618 (M+Na).

Preparation 10

A solution of benzyl (2S,6E)-9-benzyloxy-2-di-tert-butoxycarbonylamino-8-oxodec-6-enoate (2.74 g) in ethyl acetate (30 ml) was hydrogenated in the presence of 10% palladium-carbon (300 mg) for 2 hours. The reaction mixture was filtered through a pad of Celite® and concentrated in vacuo to give (2S)-9-benzyloxy-2-di-tert-butoxycarbonylamino-8-oxodecanoic acid as an oil (2.27 g).

¹H-NMR (300 MHz, CDCl₃, δ): 1.19-1.53 (6H, m), 1.33 (3H, d, J=7 Hz), 1.50 (2×9H, S), 1.89 (1H, m), 2.07 (1H, m), 2.44-2.65 (2H, m), 3.92 (1H, q, J=7 Hz), 4.48 (1H, d, J=12 Hz), 4.54 (1H, d, J=12 Hz), 4.89 (1H, dd, J=10, 5 Hz), 7.22-7.40 (5H, m);

MASS (ES−): m/e 506.

Preparation 11

To a solution of (2S)-9-benzyloxy-2-di-tert-butoxycarbonylamino-8-oxodecanoic acid (164 mg) in dioxane (2 ml) was added 4N-hydrogen chloride in dioxane (2 ml), and the mixture was stirred at ambient temperature for 3 hours. The solvent was evaporated in vacuo and the residual solvent was removed azeotropically with toluene to give (2S)-2-amino-9-benzyloxy-8-oxodecanoic acid hydrochloride as an amorphous (109 mg).

¹H-NMR (300 MHz, DMSO-d₆, δ): 1.16-1.53 (6H, m), 1.23 (3H, d, J=7 Hz), 1.76 (2H, m), 2.55 (2H, m), 3.86 (1H, t, J=5 Hz), 3.99 (1H, q, J=7 Hz), 4.46 (1H, d, J=12 Hz), 4.51 (1H, d, J=12 Hz), 7.26-7.41 (5H, m), 8.30 (2H, br);

MASS (ES+): m/e 308.

Preparation 12

To a stirred solution of (2S)-2-amino-9-benzyloxy-8-oxodecanoic acid hydrochloride (1.37 g) in dioxane (20 ml) were added 1N-sodium hydroxide (8.8 ml) and di-tert-butyldicarbonate (1.04 g) in dioxane, and the mixture was stirred at ambient temperature for 4 hours. The mixture was concentrated in vacuo. The residue was diluted with water and the mixture was washed with diethyl ether. The aqueous phase was acidified with 1N-hydrogen chloride, and extracted with ethyl acetate. The organic layer was washed with brine, dried over magnesium sulfate, and evaporated in vacuo to give (2S)-9-benzyloxy-2-tert-butoxycarbonylamino-8-oxodecanoic acid (hereinafter Compound (12)) as a colorless oil (1.48 g).

¹H-NMR (300 MHz, CDCl₃, δ): 1.21-1.46 (4H, m), 1.33 (3H, d, J=7 Hz), 1.52-1.74 (3H, m), 1.84 (1H, m), 2.55 (2H, m), 3.72 (1H, q, J=7 Hz), 4.28 (1H, m), 4.49 (1H, d, J=12 Hz), 4.55 (1H, d, J=12 Hz), 4.97 (1H, brd, J=8 Hz), 7.21-7.40 (5H, m);

MASS (ES−): m/e 406.

Preparation 13

To a stirred solution of N-tert-butoxycarbonyl-(R)-proline (50 g) in N,N-dimethylformamide (250 ml), cesium carbonate (37.8 g) was added portionwise under ice-cooling in an ice bath. The ice bath was removed and the suspension was stirred at ambient temperature for 1.5 hours. To the suspension benzyl bromide (40.9 g) was added under ice-cooling and the mixture was stirred at ambient temperature for two and half an hour. To this mixture, water (250 ml) was added under ice-cooling and the mixture was extracted with ethyl acetate (1500 ml), and washed with water (250 ml, 3 times) and brine (250 ml). The organic layer was dried over anhydrous sodium sulfate, filtered and the filtrate was concentrated in vacuo to give crude Compound (13) (N-tert-butoxycarbonyl-(R)-proline benzyl ester, 87.3 g) as a colorless oil.

¹H-NMR (300 MHz, CDCl₃, δ): 1.35 (6H, s), 1.46 (3H, s), 1.76-2.04 (3H, m), 2.07-2.31 (1H, m), 3.31-3.61 (2H, m), 4.26 (0.6H, dd, J=8.0, 3.6 Hz), 4.40 (0.4H, dd, J=8.4, 2.4 Hz), 5.04-5.30 (2H, m), 7.25-7.40 (5H, m);

MASS (ES+): m/e 306.13 (M+1).

Preparation 14

To the Compound (13) (114 mg), 4N hydrogen chloride in ethyl acetate (50 ml) was added at ambient temperature and the mixture was stirred at ambient temperature for 2 hours. The mixture was concentrated in vacuo and the residual hydrogen chloride was removed azeotropically with ethyl acetate 4 times.

The residual amorphous solid was dissolved in N,N-dimethylformamide (3 ml), and to the solution were added O-benzyl-N-tert-butoxycarbonyltyrosine (146 mg), 1-ethyl-3-(3′-N,N-dimethylaminopropyl)carbodiimide (63.8 mg) and 1-hydroxybenzotriazole (55.5 mg) under ice-cooling. The mixture was stirred at ambient temperature for 1.5 hours. The mixture was diluted with ethyl acetate (300 ml) and washed with 5% aqueous potassium hydrogensulfate solution (200 ml, 4 times), saturated aqueous sodium bicarbonate solution (300 ml, twice) and brine (300 ml). The organic layer was dried over anhydrous sodium sulfate, filtered and concentrated in vacuo. The residue was purified by flash chromatography eluted with ethyl acetate-hexane (1:1 v/v) to give Compound (14) (201 mg) as a colorless amorphous solid.

¹H-NMR (300 MHz, CDCl₃, δ): 7.45-7.25 (10H, m), 7.11 (2H, d, J=8 Hz), 6.87 (2H, d, J=8 Hz), 5.37 (1H, brd, J=8.4 Hz), 5.24-4.95 (2H, m), 4.64-4.52 (1H, m), 4.31 (1H, dd, J=7.3, 4.8 Hz), 3.55-3.45 (2H, m), 3.00 (1H, dd, J=12.8, 5.6 Hz), 2.86 (1H, dd, J=12.8, 9.6 Hz), 2.70-2.55 (1H, m), 1.92-1.70 (2H, m), 1.60 (1H, m), 1.43 (9H, s);

MASS (ES+): m/e 559.36 (M+1).

Preparation 15

To the Compound (14) (6.21 g) was added 4N hydrogen chloride in ethyl acetate (100 ml) under ice-cooling and the mixture was stirred at ambient temperature for one hour. The mixture was concentrated in vacuo and the residual hydrogen chloride was removed 4 times azeotropically with ethyl acetate.

The residual amorphous solid was dissolved in N,N-dimethylformamide (60 ml), then Compound 1 (2.42 g), PyBOP® (6.36 g) (Nova biochem, benzotriazol-1-yloxy-tris-pyrrolidinophosphonium hexafluorophosphate) and N,N-diisopropylethylamine (4.74 g) were added to this solution, and the resulting mixture was stirred at ambient temperature for 16 hours. The volatiles were removed in vacuo and the residue was extracted with ethyl acetate (500 ml).

The organic layer was washed with aqueous 5% potassium hydrogensulfate solution (100 ml, 4 times), saturated aqueous sodium bicarbonate solution (100 ml, 4 times), water (100 ml) and brine (100 ml). The organic layer was separated, dried over anhydrous sodium sulfate, filtered and concentrated in vacuo. The residue was purified by flash chromatography (eluting with ethyl acetate/hexane=2:1 v/v) to give Compound (15) (5.10 g) as an amorphous solid.

¹H-NMR (300 MHz, CDCl₃, δ): 7.55-7.20 (10H, m), 7.10 (2H, d, J=7.6 Hz), 7.00-6.73 (3H, m), 5.20-4.96 (3H, m), 4.94-4.80 (1H, m), 4.49-4.30 (1H, m), 3.61-3.44 (2H, m), 3.00 (1H, dd, J=13.0, 5.4 Hz), 2.86 (1H, dd, J=13.0, 8.8 Hz), 2.75-2.60 (1H, m), 2.06-1.35 (5H, m), 1.43 (9H, s), 0.80 (3H, t, J=6.3 Hz);

MASS (ES+): m/e 658.43 (M+1).

Preparation 16

To the Compound (15) (5.59 g) was added 4N hydrogen chloride in ethyl acetate (50 ml) under ice-cooling and the mixture was stirred at ambient temperature for 1 hour. The mixture was concentrated in vacuo and the residual hydrogen chloride was removed 4 times azeotropically with ethyl acetate.

The residue was dissolved in dichloromethane (50 ml) and to this solution was added Compound b (3.14 g), PyBOP® (4.86 g) and N,N-diisopropylethylamine (3.62 g) under ice-cooling, and the resulting mixture was stirred at ambient temperature for 16 hours. The volatiles were removed in vacuo and the residue was extracted with ethyl acetate (500 ml). The organic layer was washed with 5% aqueous potassium hydrogensulfate solution (200 ml, 4 times), saturated aqueous sodium bicarbonate solution (200 ml, twice), water (200 ml, twice) and brine (100 ml). The residue was purified by flash chromatography (eluting with ethyl acetate/hexane=2:1 v/v) to give Compound (16) (5.2 g) as a colorless amorphous solid.

¹H-NMR (300 MHz, CDCl₃, δ): 8.10-7.98 (2H, m), 7.60-7.22 (13H, m), 7.14-6.77 (5H, m), 6.69 (1H, brd, J=6.7 Hz), 5.18-4.95 (5H, m), 4.93-4.83 (1H, m), 4.39-4.32 (1H, m), 4.31 (2H, t, J=6.6 Hz), 4.12-4.02 (1H, m), 3.61-3.49 (2H, m), 3.03-2.85 (2H, m), 2.82-2.70 (1H, m), 2.36-2.19 (1H, m), 1.98-1.38 (10H, m), 1.50 (3H, s), 1.44 (9H, s), 0.72 (3H, t, J=7.3 Hz);

MASS (ES+): m/e 891.49 (M).

Preparation 17

A solution of the Compound (16) (5.43 g) in ethyl acetate (110 ml) was hydrogenated in the presence of palladium hydroxide and 20 wt % Pd (dry basis) on carbon (Pearlman's catalyst) (540 mg) for 4 hours under atmosphere pressure. The catalyst was filtered off through a pad of Celite® and the filtrate was concentrated in vacuo. The residue was purified by flash column chromatography eluting with chloroform/methanol=10:1 v/v to give Compound (17) as a colorless amorphous (4.96 g).

¹H-NMR (300 MHz, CDCl₃, δ): 0.78 (3H, t, J=7.0 Hz), 1.44 (9H, s), 1.30-2.00 (13H, m), 2.06-2.19 (1H, m), 2.64-2.77 (1H, m), 2.95 (2H, brd, J=6.6 Hz), 3.55-3.69 (1H, m), 3.94-4.07 (1H, m), 4.25-4.38 (3H, m), 4.87 (1H, m), 5.05 (2H, s), 6.82 (1H, s), 6.87 (2H, d, J=8.5 Hz), 7.11 (2H, d, J=8.5 Hz), 7.20 (1H, brd, J=8.8 Hz), 7.27-7.60 (8H, m), 7.99-8.07 (2H, m);

MASS (ES+): m/e 801.47 (M+1).

Preparation 18

To the Compound (17) (4.96 g) was added 4N hydrogen chloride in ethyl acetate (60 ml) under ice-cooling and the mixture was stirred at ambient temperature for 3 hours. The solvent was concentrated in vacuo and the residual hydrogen chloride was removed azeotropically with ethyl acetate (100 ml, 4 times). The residue was dried in vacuo to give Compound (18) (4.64 g) as a pale brown amorphous solid. The obtained compound was used in the Preparation 75.

¹H-NMR (300 MHz, CDCl₃, δ): 0.60-0.82 (3H, m), 1.25-2.20 (15H, m), 2.74-3.07 (4H, m), 3.63-3.79 (1H, m), 4.13-4.38 (3H, m), 4.82-4.95 (1H, m), 4.99 (2H, s), 6.83 (2H, d, J=7.3 Hz), 7.10 (2H, d, J=7.3 Hz), 7.20-7.54 (8H, m), 7.51 (1H, t, J=8.1 Hz), 7.57-7.70 (1H, m), 7.99 (2H, d, J=7.0 Hz), 8.07-8.40 (2H, m);

MASS (ES+): m/e 701.36 (free+1).

Preparation 19

The Compound (13) (10.0 g) was dissolved in ethyl acetate (60 ml) and the mixture was stirred for 4 hours at ambient temperature. The solvent was evaporated and the residual solvent was removed azeotropically with toluene. The residue was washed with ethyl acetate and dried to give D-proline benzyl ester hydrochloride (hereinafter Compound 19).

¹H-NMR (300 MHz, CDCl₃, δ): 1.92 (2H, m), 2.01 (1H, m), 2.28 (1H, m), 3.22 (1H, m), 4.44 (1H, dd, J=8, 7 Hz), 5.23 (1H, d, J=12 Hz), 5.26 (1H, d, J=12 Hz), 7.23-7.47 (5H, m);

MASS (ES+): m/e 206.

Preparation 20

N-t-Butoxycarbonyl O-methyl-L-tyrosine (3.62 g) was dissolved in dichloromethane (40 ml), then Compound 19 (2.82 g), hydroxybenzotriazol (1.73 g) and a solution of 1-ethyl-3-(3′-dimethylaminopropyl)carbodiimide hydrogen chloride (1.99 g) in dichloromethane (5 ml) were added to the mixture and the mixture was stirred for 14 hours at ambient temperature. The reaction mixture was added to 10% aqueous solution of citric acid (50 ml) then 5% aqueous solution of potassium hydrogensulfate (50 ml) was added to the mixture. The mixture was washed with saturated aqueous sodium bicarbonate solution (50 ml) and saturated aqueous sodium chloride solution (50 ml) then dried over magnesium sulfate, and evaporated to dryness to give a crude compound. The crude compound was purified by flash column chromatography (Silica gel 60, Spherical, 120 g, eluent: ethyl acetate:hexane=1:2 to 1:1) to give Compound (20) (5.55 g).

¹H-NMR (300 MHz, CDCl₃, δ): 1.43 (3×3H, s), 1.55 (1H, m), 1.74-2.00 (3H, m), 2.69 (1H, m), 2.87 (1H, dd, J=13.9 Hz), 3.00 (1H, dd, J=13, 5 Hz), 3.54 (1H, m), 4.36 (1H, dd, J=8, 4 Hz), 4.60 (1H, m), 5.11 (1H, d, J=12.5 Hz), 5.19 (1H, d, J=12.5 Hz), 5.37 (1H, d, J=9 Hz), 6.79 (2×1H, d, J=8.5 Hz), 7.12 (2×1H, d, J=8.5 Hz), 7.28-7.40 (5H, m);

MASS (ES+): m/e 483.

Preparation 21

The Compound (20) (5.50 g) was dissolved in ethyl acetate (30 ml) and a cold solution of 4N hydrogen chloride in ethyl acetate (50 ml) was added to the mixture and stirred for 2.5 hours at ambient temperature. The mixture was evaporated to dryness to give Compound (21) (4.97 g) as a white foam.

¹H-NMR (300 MHz, CDCl₃, δ): 1.60 (1H, m), 1.70-1.87 (2H, m), 1.97 (1H, m), 2.80 (1H, m), 2.91 (1H, dd, J=13, 8 Hz), 3.06 (1H, dd, J=13, 6 Hz), 3.58 (1H, m), 4.30 (1H, dd, J=9, 3 Hz), 4.36 (1H, m), 5.08 (1H, d, J=13 Hz), 5.19 (1H, d, J=13 Hz), 6.90 (2×1H, d, J=8 Hz), 7.14 (2×1H, d, J=8 Hz), 7.30-7.44 (5H, m), 8.34 (2H, br);

MS (ES+): m/e 383.

Preparation 22

The Compound (21) (4.89 g) was dissolved in dichloromethane (40 ml) and Compound a (4.31 g), benzotriazol-1-yloxy-tris-pyrrolidinophosphonium hexafluorophosphate (6.68 g) and N-ethyldiisopropylamine (4.83 g) were added to the solution, and the mixture was stirred for 15 hours at ambient temperature. The mixture was diluted with chloroform (40 ml), washed with 5% aqueous solution of potassium hydrogensulfate (50 ml), saturated aqueous sodium bicarbonate solution (50 ml) and saturated aqueous sodium chloride solution (50 ml), dried over sodium sulfate and evaporated to give a crude compound. The crude compound was purified by flash column chromatography (Silica gel 60N, Spherical, 120 g, eluent: ethyl acetate:hexane=1:2 to 1:1) to give Compound (22) (5.70 g).

¹H-NMR (300 MHz, CDCl₃, δ): 0.81 (3H, t, J=7.5 Hz), 1, 41 (3H, s), 1, 44 (9H, s), 1.58 (1H, m), 1.76-2.06 (5H, m), 2.75 (1H, m), 2.89 (1H, dd, J=13, 9 Hz), 3.02 (1H, dd, J=13, 5 Hz), 3.56 (1H, m), 3.77 (3H, s), 4.38 (1H, dd, J=8, 4 Hz), 4.91 (1H, ddd, J=9, 8.5, 5 Hz), 5.11 (1H, d, J=12.5 Hz), 5.15 (1H, d, J=12.5 Hz), 6.80 (2H, d, J=8.5 Hz), 6.84 (1H, d, J=8.5 Hz), 7.13 (2H, d, J=8.5 Hz), 7.28-7.40 (5H, m);

MASS (ES+): m/e 582.

Preparation 23

The Compound (22) (5.31 g) was dissolved in ethyl acetate (30 ml) and cold solution of 4N hydrogen chloride in ethyl acetate (50 ml) was added to the mixture and stirred for 1 hour at ambient temperature. The mixture was evaporated to dryness to give Compound (23) (5.31 g) as a white foam.

¹H-NMR (300 MHz, CDCl₃, δ): 0.75 (3H, d, J=7 Hz), 1.33 (3H, s), 1.63-2.30 (6H, m), 2.84 (1H, dd, J=13, 10 Hz), 2.93 (1H, dd, J=13, 5 Hz), 3.51 (1H, m), 3.74 (1H, m), 4.34 (1H, dd, J=9, 4 Hz), 4.80 (1H, ddd, J=9 Hz), 7.20 (2×1H, d, J=9 Hz), 7.29-7.45 (1H, m), 8.03 (2H, brs), 8.64 (1H, d, J=9 Hz);

MS (ES+): m/e 482.

Preparation 24

The Compound (23) (5.26 g) was dissolved in dichloromethane (30 ml) and a solution of Compound (5) (3.57 g) in dichloromethane (50 ml), benzotriazol-1-yloxy-tris-pyrrolidinophosphonium hexafluorophosphate (6.34 g) and N-ethyldiisopropylamine (4.2 g) were added to the solution, and the mixture was stirred for 12 hours at ambient temperature. The mixture was diluted with chloroform (80 ml), washed with 5% aqueous solution of potassium hydrogensulfate (100 ml), saturated aqueous sodium bicarbonate solution (100 ml) and saturated aqueous sodium chloride solution (100 ml), dried over sodium sulfate and evaporated to give a crude compound. The crude compound was purified by flash column chromatography (Silica gel 60N, Spherical, 150 g, eluent: ethyl acetate:hexane=1:1 to 1:2) to give Compound (24) (5.76 g).

¹H-NMR (300 MHz, CDCl₃, δ): 0.72 (3H, t, J=7.3 Hz), 1.43 (3H, s), 1.44 (3×3H, s), 1.47-2.36 (12H, m), 2.84 (1H, m), 2.92 (1H, dd, J=13, 9.5 Hz), 2.98 (1H, dd, J=13, 5.5 Hz), 3.58 (1H, m), 3.77 (3H, s), 4.08 (1H, m), 4.32 (2H, t, J=6.5 Hz), 4.39 (1H, dd, J=8, 4 Hz), 4.91 (1H, m), 5.12 (1H, m), 5.13 (2H, s), 6.70 (1H, brd, J=9 Hz), 6.80 (2×1H, d, J=8.5 Hz), 7.01 (1H, s), 7.10 (2×1H, d, J=8.5 Hz), 7.28-7.36 (5H, m), 7.43 (2×1H, dd, J=7.5, 7.5 Hz), 7.55 (1H, dd, J=7.5, 7.5 Hz), 8.03 (2×1H, d, J=7.5 Hz);

MASS (ES−): m/e 813.

Preparation 25

Compound (25) was obtained in a manner similar to Pteparation 17 except that Compound (24) was used instead of the Compound (16) and palladium on carbon was used instead of the Pearlman's catalyst.

¹H-NMR (300 MHz, CDCl₃, δ): 0.77 (3H, t, J=7.5 Hz), 1.38-2.36 (12H, m), 1.44 (9+3H, s), 2.79 (1H, m), 2.90-3.02 (2H, m), 3.67 (1H, m), 3.77 (3H, s), 4.02 (1H, m), 4.26-4.42 (3H, m), 4.88 (1H, m), 5.20 (1H, m), 6.81 (2×1H, d, J=8.5 Hz), 6.83 (1H, brs), 7.12 (2×1H, d, J=8.5 Hz), 7.24 (1H, d, J=8 Hz), 7.43 (2×1H, dd, J=7.5, 7.5 Hz), 7.56 (1H, dd, J=7.5, 7.5 Hz), 8.04 (2×1H, d, J=7.5 Hz);

MASS (ES−): m/e 723.

Preparation 26

Compound (26) was obtained in a manner similar to Preparation 18 except that trifluoroacetic acid was used instead of 4N hydrogen chloride. The obtained compound was used in Preparation 78.

¹H-NMR (300 MHz, DMSO-d₆, δ): 0.54 (3×⅓H, t, J=7.3 Hz, 0.66 (3×⅔H, t, J=7.3 Hz), 1.31 (3×⅓H, s), 1.35 (3×⅔H, s), 1.44 (2H, m), 1.60-2.20 (10H, m), 2.70-2.98 (2H, m), 3.18 (1H, m), 3.36 (1H, m), 3.67 (3×⅓H, s), 3.69 (3×⅔H, s), 4.12 (1×⅔H, dd, J=9.3 Hz), 4.26 (2H, t, J=6 Hz), 4.41 (1H, m), 4.77 (1H, m), 4.84 (1×⅓H, dd, J=9.3 Hz), 6.78 (2×⅓H, d, J=9 Hz), 6.81 (2×⅔H, d, J=9 Hz), 7.10-7.30 (3H, m), 7.48-7.60 (2H, m), 7.68 (1H, m), 7.88-8.17 (5H, m);

MASS (ES+): m/e 625.

Preparation 27

Compound (27) was obtained in a manner similar to Preparation (14).

¹H-NMR (300 MHz, CDCl₃, δ): 1.42 (9H, s), 1.50-1.68 (1H, m), 1.80-2.03 (3H, m), 2.71-2.84 (1H, m), 2.92 (1H, dd, J=13.2, 8.7 Hz), 3.00 (1H, dd, J=13.2, 6.1 Hz), 3.53-3.65 (1H, m), 4.36 (1H, dd, J=7.7, 3.6 Hz), 4.62 (1H, dt, J=8.5, 5.9 Hz), 5.10 (1H, d, J=12.5 Hz), 5.20 (1H, d, J=12.5 Hz), 5.34 (1H, d, J=8.0 Hz), 6.88-7.03 (2H, m), 7.17 (2H, dd, J=8.5, 5.5 Hz), 7.30-7.40 (5H, m);

MASS (ES+): m/e 471.37 (M+1).

Preparation 28

Compound (28) was obtained in a manner similar to Preparation 15.

¹H-NMR (300 MHz, CDCl₃, δ): 0.80 (3H, t, J=7.6 Hz), 1.39 (3H, s), 1.43 (9H, s), 1.76-2.03 (6H, m), 2.74-2.87 (1H, m), 2.95 (1H, dd, J=13.2 and 9.1 Hz), 3.03 (1H, dd, J=13.2 and 4.8 Hz), 3.51-3.66 (1H, m), 4.38 (1H, dd, J=8.1, 3.7 Hz), 4.87-4.98 (1H, m), 4.98-5.20 (3H, m), 6.81-7.02 (3H, m), 7.15-7.23 (2H, m), 7.28-7.41 (5H, m);

MASS (ES+): m/e 570.42 (M+1).

Preparation 29

Compound (29) was obtained in a manner similar to Preparation 15.

¹H-NMR (300 MHz, CDCl₃, δ): 0.61 (0.6H, t, J=7.3 Hz), 0.72 (2.4H, t, J=7.3 Hz), 1.39-2.08 (11H, m), 1.43 (9H, s), 1.48 (3H, s), 2.13-2.33 (1H, m), 2.83-2.99 (1H, m), 2.98 (2H, d, J=7.0 Hz), 3.51-3.70 (1H, m), 3.92-4.15 (1H, m), 4.31 (2H, t, J=5.9 Hz), 4.39 (1H, dd, J=7.3, 3.2 Hz), 4.92 (1H, q, J=7.3 Hz), 5.02-5.15 (2H, m), 5.17 (1H, s), 6.72 (1H, brs), 6.83-7.05 (3H, m), 7.16 (2H, dd, J=8.4, 5.5 Hz), 7.27-7.38 (5H, m), 7.39-7.47 (2H, m), 7.51-7.60 (1H, m), 8.03 (2H, d, J=7.3 Hz);

MASS (ES+): m/e 803.55 (M+1).

Preparation 30

Compound (30) was obtained in a manner similar to Preparation 17.

¹H-NMR (300 MHz, CDCl₃, δ): 0.77 (3H, t, J=7.4 Hz), 1.17-2.02 (11H; m), 1.45 (12H, s), 2.11-2.25 (1H, m), 2.79-3.10 (3H, m), 3.64-3.79 (1H, m), 4.26-4.42 (3H, m), 4.92 (1H, q, J=7.6 Hz), 5.23 (1H, brs), 6.79 (1H, brs), 6.97 (2H, t, J=8.5 Hz), 7.19 (2H, dd, J=8.5, 5.2 Hz), 7.30 (1H, d, J=8.3 Hz), 7.39-7.48 (2H, m), 7.52-7.62 (1H, m), 8.04 (2H, d, J=8.5 Hz);

MASS (ES+): m/e 713.54 (M+1).

Preparation 31

Compound (31) was obtained in a manner similar to Preparation 18. The obtained compound was used in Preparation 81.

¹H-NMR (300 MHz, CDCl₃, δ): 0.70 (3H, t, J=7.4 Hz), 1.38 (3H, s), 1.51-2.16 (12H, m), 2.83-3.15 (3H, m), 3.68-3.83 (1H, m), 4.18-4.37 (4H, m), 4.86-4.98, (1H, m), 6.92 (2H, t, J=8.5 Hz), 7.17 (2H, dd, J=8.5, 5.8 Hz), 7.39 (2H, t, J=7.7 Hz), 7.53 (1H, t, J=7.6 Hz), 7.67 (1H, brs), 7.99 (2H, d, J=7.3 Hz), 8.13-8.39 (3H, m);

MASS (ES+): m/e 613.49 (M+1, free).

Preparation 32

Compound (32) was obtained in a manner similar to Preparation 14.

Preparation 33

Compound (33) was obtained in a manner similar to Preparation 15.

¹H-NMR (300 MHz, CDCl₃, δ): 1.31-1.54 (9H, m), 1.55-1.99 (8H, m), 2.01-2.42 (3H, m), 2.52-2.63 (1H, m), 2.80-2.96 (1H, m), 3.03-3.14 (1H, m), 3.44-3.60 (2H, m), 4.31-4.38 (1H, m), 4.68-4.86 (1H, m), 4.94 (1H, dt, J=9.9, 5.1 Hz), 5.05-5.20 (2H, m), 7.08 (1H, d, J=8.1 Hz), 7.16-7.39 (10H, m);

MASS (ES+): m/e 564.38 (M+1).

Preparation 34

Compound (34) was obtained in a manner similar to Preparation 16.

¹H-NMR (300 MHz, CDCl₃, δ): 1.32-2.06 (20H, m), 1.44 (9H, s), 2.09-2.30 (2H, m), 2.64-2.74 (1H, m), 2.88-3.08 (1H, m), 3.53-3.62 (2H, m), 3.98-4.08 (1H, m), 4.27-4.37 (4H, m), 4.85-4.95 (1H, m), 5.07-5.21 (3H, m), 6.63 (1H, s), 7.12-7.37 (6H, m), 7.42 (2H, dd, J=8.1, 6.9 Hz), 7.55 (1H, dd, J=6.9, 6.9 Hz), 8.03 (2H, d, J=8.1 Hz);

MASS (ES+): m/e 797.50 (M+1).

Preparation 35

Compound (35) was obtained in a manner similar to Preparation 17.

¹H-NMR (300 MHz, CDCl₃, δ): 1.16-2.12 (15H, m), 1.44 (9H, s), 2.24-2.41 (1H, m), 2.62-2.76 (1H, m), 2.90-3.09 (2H, m), 3.47-3.50 (1H, m), 3.65-3.77 (1H, m), 4.01-4.11 (2H, m), 4.24-4.38 (4H, m), 4.74-4.84 (1H, m), 5.56-5.64 (1H, m), 6.84-6.92 (1H, m), 7.16-7.31 (6H, m), 7.43 (2H, dd, J=7.8, 6.9 Hz), 7.56 (1H, dd, J=7.8, 7.8 Hz), 8.02 (2H, d, J=6.9 Hz);

MASS (ES+): m/e 707.45 (M+1).

Preparation 36

Compound (36) was obtained in a manner similar to Preparation 18. The obtained compound was used in Preparation 84.

¹H-NMR (300 MHz, CDCl₃, δ): 1.34-2.27 (19H, m), 2.79-3.19 (3H, m), 3.48-3.78 (1H, m), 3.95-4.13 (1H, m), 4.14-4.47 (3H, m), 4.82-5.00 (1H, m), 7.11-7.32 (5H, m), 7.34-7.46 (2H, m), 7.48-7.58 (1H, m), 7.62-7.84 (1H, brs), 7.95-8.06 (2H, m), 8.06-8.36 (2H, brs), 8.63-9.02 (1H, brs);

MASS (ES+): m/e 607.42 (M+1).

Preparation 37

Compound (37) was obtained in a manner similar to Preparation 19.

¹H-NMR (300 MHz, CDCl₃, δ): 1.31 (3H, s), 1.40 (6H, s), 1.56-1.80 (3H, m), 1.84-2.11 (2H, m), 2.92-3.13 (2H, m), 3.57-3.70 (1H, m), 4.36-4.42 (1H, m), 4.62-4.72 (1H, m), 5.04-5.34 (3H, m), 7.11-7.51 (7H, m), 7.54-7.60 (3H, m);

MASS (ES+): m/e 478.40 (M+1).

Preparation 38

Compound (38) was obtained in a manner similar to Preparation 15.

¹H-NMR (300 MHz, CDCl₃, δ): 0.800 (3H, t, J=7.5 Hz), 1.36 (3H, s), 1.39 (3H, s), 1.43 (6H, s), 1.52-1.62 (2H, m), 1.67-2.06 (4H, m), 2.83-3.16 (2H, m), 3.50-3.70 (2H, m), 4.36-4.42 (1H, m), 4.86-5.04 (2H, m), 5.06-5.21 (2H, m), 6.87 (1H, d, J=9.0 Hz), 7.29-7.48 (6H, m), 7.53-7.59 (3H, m);

MASS (ES+): m/e 577.40 (M+1).

Preparation 39

Compound (39) was obtained in a manner similar to Preparation 16.

¹H-NMR (300 MHz, CDCl₃, δ): 0.740 (3H, t, J=7.2 Hz), 1.30-2.29 (11H, m), 1.34 (3H, s), 1.44 (9H, s), 2.86-3.18 (3H, m), 3.51-3.72 (2H, m), 3.99-4.08 (1H, m), 4.27-4.42 (3H, m), 4.96-5.04 (1H, m), 5.06-5.19 (3H, m), 6.82 (1H, s), 7.12-7.17 (1H, m), 7.28-7.37 (6H, m), 7.39-7.47 (3H, m), 7.52-7.61 (3H, m), 8.00-8.05 (2H, m);

MASS (ES+): m/e 810.59 (M+1).

Preparation 40

Compound (40) was obtained in a manner similar to Preparation 17.

¹H-NMR (300 MHz, CDCl₃, δ): 0.731 (3H, t, J=7.2 Hz), 1.31-2.22 (13H, m), 1.40 (3H, s), 1.44 (9H, s), 2.91-3.23 (3H, m), 3.80-3.94 (1H, m), 3.99-4.13 (1H, m), 4.23-4.43 (3H, m), 4.86-5.00 (1H, m), 5.48-5.60 (1H, m), 6.76 (1H, s), 7.25-7.31 (1H, m), 7.31-7.38 (2H, m), 7.40-7.47 (2H, m), 7.52-7.61 (3H, m), 8.00-8.06 (2H, m);

MASS (ES+): m/e 720.38 (M+1).

Preparation 41

Compound (41) was obtained in a manner similar to Preparation 18. The obtained compound was used in Preparation C5.

¹H-NMR (300 MHz, CDCl₃, δ): 0.59-0.73 (3H, m), 1.33 (3H, s), 1.52-2.17 (12H, m), 2.92-3.27 (3H, m), 3.70-3.83 (1H, m), 4.14-4.40 (4H, m), 4.90-5.02 (1H, m), 7.31-7.45 (5H, m), 7.49-7.59 (3H, m), 7.59-7.71 (1H, brs), 7.93-8.11 (5H, m);

MASS (ES+): m/e 620.33 (M+1).

Preparation 42

Compound (42) was obtained in a manner similar to Preparation 19.

¹H-NMR (300 MHz, CDCl₃, δ): 1.39 (3H, t, J=7.2 Hz), 1.43 (9H, s), 1.46-1.63 (1H, m), 1.76-2.00 (3H, m), 2.62-2.72 (1H, m), 2.82-2.92 (1H, m), 2.94-3.04 (1H, m), 3.48-3.58 (1H, m), 3.98 (2H, q, J=7.2 Hz), 4.32-4.42 (1H, m), 4.53-4.64 (1H, m), 5.10 (1H, d, J=12.6 Hz), 5.20 (1H, d, J=12.6 Hz), 5.37 (1H, d, J=8.7 Hz), 6.78 (2H, d, J=8.7 Hz), 7.11 (2H, d, J=8.7 Hz), 7.28-7.39 (5H, m);

MASS (ES+): m/e 497.34 (M+1).

Preparation 43

Compound (43) was obtained in a manner similar to Preparation 15.

¹H-NMR (300 MHz, CDCl₃, δ): 0.80 (3H, t, J=7.5 Hz), 1.39 (3H, t, J=7.2 Hz), 1.40 (3H, s), 1.43 (9H, s), 1.50-1.64 (1H, m), 1.75-2.05 (5H, m), 2.67-2.79 (1H, m), 2.81-2.93 (1H, m), 2.94-3.05 (1H, m), 3.50-3.62 (1H, m), 3.98 (2H, q, J=7.2 Hz), 4.37 (1H, dd, J=7.5, 3.3 Hz), 4.90 (1H, dt, J=9.6, 5.1 Hz), 5.10 (1H, d, J=12.3 Hz), 5.15 (1H, d, J=12.3 Hz), 6.57-6.97 (1H, m), 6.78 (2H, d, J=8.4 Hz), 7.11 (2H, d, J=8.4 Hz), 7.29-7.39 (5H, m);

MASS (ES+): m/e 596.51 (M+1).

Preparation 44

Compound (44) was obtained in a manner similar to Preparation 16.

¹H-NMR (300 MHz, CDCl₃, δ): 0.74 (3H, t, J=7.5 Hz), 1.40 (3H, t, J=7.2 Hz), 1.45 (9H, s), 1.47-1.99 (11H, m), 1.50 (3H, s), 2.18-2.29 (1H, m), 2.76-3.00 (2H, m), 3.44-3.65 (2H, m), 3.99 (2H, q, J=7.2 Hz), 4.03-4.13 (1H, m), 4.33 (2H, t, J=6.3 Hz), 4.40 (1H, dd, J=7.2, 3.6 Hz), 4.83-4.94 (1H, m), 5.10-5.19 (3H, m), 6.79 (2H, d, J=8.4 Hz), 6.92-7.04 (1H, m), 7.10 (2H, d, J=8.4 Hz), 7.29-7.39 (6H, m), 7.40-7.48 (2H, m), 7.52-7.60 (1H, m), 8.01-8.07 (2H, m);

MASS (ES+): m/e 829.61 (M+1).

Preparation 45

Compound (45) was obtained in a manner similar to Preparation 17.

¹H-NMR (300 MHz, CDCl₃, δ): 0.79 (3H, t, J=7.5 Hz), 1.40 (3H, t, J=7.2 Hz), 1.44 (12H, s), 1.57-2.72 (11H, m), 2.65-3.03 (3H, m), 3.58-3.83 (2H, m), 3.99 (2H, q, J=7.2 Hz), 4.04-4.15 (1H, m), 4.23-4.39 (3H, m), 4.75-4.88 (1H, m), 5.53-5.63 (1H, m), 6.79 (2H, d, J=8.7 Hz), 7.09 (2H, d, J=8.7 Hz), 7.13-7.21 (1H, m), 7.39-7.48 (2H, m), 7.52-7.59 (1H, m), 8.00-8.06 (2H, m);

MASS (ES+): m/e 739.58 (M+1).

Preparation 46

Compound (46) was obtained in a manner similar to Preparation 18. The obtained compound was used in Preparation 91.

¹H-NMR (300 MHz, CDCl₃, δ): 0.72 (3H, t, J=6.9 Hz), 1.34 (3H, s), 1.38 (3H, t, J=7.2 Hz), 1.54-2.13 (12H, m), 2.80-3.18 (3H, m), 3.64-3.78 (1H, m), 3.36 (2H, q, J=7.2 Hz), 4.14-4.38 (4H, m), 4.77-4.89 (1H, m), 6.77 (2H, d, J=8.7 Hz), 7.09 (2H, d, J=8.7 Hz), 7.37-7.48 (2H, m), 7.49-7.57 (1H, m), 7.80-8.22 (6H, m);

MASS (ES+): m/e 739.58 (free M+1).

Preparation 47

Compound (47) was purchased from Kokusan Chemical Co., Ltd.

Preparation 48

Fmoc-2-fluorophenylalanine (available from Oakwood Products, Inc.), 1-ethyl-3-(3′-dimethylaminopropyl)carbodiimide hydrochloride (1.29 g) and 1-hydroxybenzotriazole (911 mg) were added to dichloroethane (30 ml), and the mixture was sonicated to give a homogeneous mixture. To this mixture, Compound (47) (1.05 g) in dichloromethane (10 ml) was added and stirred at ambient temperature for 1.3 hours. The reaction mixture was added to 10% aqueous citric acid (30 ml), then the organic layer was collected. To the aqueous layer was added water (30 ml), then the mixture was extracted with chloroform (50 ml). The organic layer and the chloroform extract were combined, washed with saturated aqueous sodium bicarbonate solution (30 ml) and brine (30 ml), dried over magnesium sulfate, and the solvent was evaporated to give a crude compound. The crude compound was purified by flash column chromatography (Silica gel 60, Spherical, 40 g, eluted with ethyl acetate/hexane=1:2 to 1:1 v/v) to give Compound (48) (3.29 g) as a white foam.

¹H-NMR (300 MHz, CDCl₃, δ): 1.43 (9×⅘H, s), 1.51 (9×⅕H, s), 1.63-2.30 (4H, m), 3.00-3.14 (2H, m), 3.20 (1H, m), 3.70 (1H, m), 4.04-4.42 (4H, m), 4.58 (1×⅕H, m), 4.82 (1×⅘H, m), 5.48 (1×⅘H, d, J=8 Hz), 5.71 (1×⅕H, d, J=8 Hz), 6.95-7.08 (2H, m), 7.11-7.62 (8H, m), 7.71-7.80 (2H, m);

MASS (ES+): m/e 559.

Preparation 49

The Compound (48) (3.25 g) was dissolved in acetonitrile (15 ml), N,N-diethylamine (15 ml) was added to the mixture and stirred for 1 hour at ambient temperature. The solvent was evaporated and the residual solvent was removed azeotropically with toluene to give a crude compound. The crude compound was purified by flash column chromatography (Silica gel 60, Spherical, 40-50 μm, eluted with methanol/chloroform=1:40 v/v) to give Compound (49) (1.52 g) as a white foam.

¹H-NMR (300 MHz, CDCl₃, δ): 1.46 (9×⅚H, s), 1.47 (9×⅙H, 6), 1.56-2.25 (4H, m), 2.79 (1×⅙H, dd, J=13, 8 Hz), 2.83 (1×⅚H, dd, J=13, 8 Hz), 2.94 (1×⅚H, dd, J=13, 7 Hz), 3.10 (1×⅙H, dd, J=13, 5 Hz), 3.19 (1H, m), 3.62 (1H, m), 3.83 (1H, d, J=8, 7 Hz), 4.28 (1×⅚H, dd, J=8, 4 Hz), 4.60 (1×⅙H, dd, J=8, 3 Hz), 6.98-7.12 (2H, m), 7.17-7.28 (2H, m);

MASS (ES+): m/e 337.

Preparation 50

The Compound (49) (1.51 g) was dissolved in dichloromethane (20 ml) and 2(S)-ethyl-2-benzyloxycarbonylaminopropionic acid (1.13 g), PyBroP® (2.3 g) and N-ethyl-N,N-diisopropylamine (696 mg) were added to the solution, and the mixture was stirred for 5 hours at ambient temperature. The mixture was washed with 10% aqueous solution of citric acid (30 ml), saturated aqueous sodium bicarbonate solution (30 ml) and saturated aqueous sodium chloride solution (30 ml), dried over magnesium sulfate and the solvent was evaporated to give a crude compound. The crude compound was purified by flash column chromatography (Silica gel 60N, Spherical, 40 g, eluent: ethyl acetate:hexane=1:2 to 1:1) to give Compound (50) (1.54 g).

¹H-NMR (300 MHz, CDCl₃, δ): 0.60 (3×¼H, t, J=7 Hz), 0.75 (3×¾H, t, J=7.3 Hz), 1.33-2.30 (18H, m), 2.98-3.32 (3H, m), 3.50-3.80 (1H, m), 4.25 (1×¾H, dd, J=8, 4 Hz), 4.67-5.10 (3+¼H, m), 5.53 (1×¼H, br), 5.78 (1×¾H, br), 6.57 (1×¼H, br), 6.73 (1×¾H, brd, J=8 Hz), 6.94-7.07 (2H, m), 7.11-7.24 (2H, m), 7.28-7.39 (5H, m);

MASS (ES+): m/e 570.

Preparation 51

The Compound (50) (1.52 g) was dissolved in methanol and 10% palladium on carbon (150 mg) suspended in water (1 ml) was added to the solution and stirred for 2 hours at ambient temperature, 3 atm. The catalyst was filtered off through a pad of Celite®, the solvent was evaporated, then the residual solvent was removed azeotropically with toluene to give Compound (51).

¹H-NMR (300 MHz, CDCl₃, δ): 0.42 (3×⅓H, t, J=7.4 Hz), 0.72 (3×⅔H, t, J=7.5 Hz), 1.19 (3×⅓H, s), 1.26 (3×⅔H, s), 1.43 (9×⅔H, s), 1.51 (9×⅓H, s), 1.69-2.30 (6H, m), 2.99-3.30 (3H, m), 3.56-3.77 (1H, m), 4.25 (1×⅔H, dd, J=8 and 4 Hz), 4.71 (1×⅓H, m), 5.02 (1×⅔H, m), 5.04 (1×⅓H, m), 6.93-7.08 (2H, m), 7.12-7.25 (2H, m);

MASS (ES+): m/e 436.

Preparation 52

The Compound (51) (1.15 g) was dissolved in dichloromethane (15 ml) and a solution of Compound (5) (1.02 g) in dichloromethane (10 ml), benzotriazole-1-yloxy-tris-pyrrolidinophosphonium hexafluorophosphate (1.65 g) and N-ethyl-N,N-diisopropylamine (751 mg) were added to the solution, and the mixture was stirred for 14 hours at ambient temperature. The mixture was washed with 10% aqueous solution of citric acid (30 ml), saturated aqueous sodium bicarbonate solution (30 ml) and saturated aqueous sodium chloride solution (30 ml), dried over sodium sulfate and the solvent was evaporated to give a crude compound. The crude compound was purified by flash column chromatography (Silica gel 60, Spherical, 50 g, eluent: ethyl acetate:hexane=1:1 to 2:1) to give Compound (52) (1.74 g) as a white foam.

¹H-NMR (300 MHz, CDCl₃, δ): 0.60 (3×⅓H, t, J=7.5 Hz), 0.71 (3×⅔H, t, J=7.5 Hz), 1.34-2.44 (12H, m), 1.41 (9×⅔H, s), 1.43 (9×⅓H, s), 1.49 (3×⅓H, s), 1.51 (3×⅔H, s), 3.00-3.12 (2H, m), 3.23-3.76 (2H, m), 4.07 (1H, m), 4.25 (1H, dd, J=8, 4 Hz), 4.31 (2H, t, J=6.5 Hz), 4.67-5.17 (2H, m), 6.54 (1×⅓H, brd, J=8 Hz), 6.70 (1×⅔H, brd, J=8 Hz), 6.93-7.09 (3H, m), 7.10-7.25 (2H, m), 7.43 (2H, dd, J=7.5, 7.5 Hz), 7.56 (1H, dd, J=7.5, 7.5 Hz), 8.03 (2H, d, J=7.5 Hz);

MASS (ES−): m/e 767.

Preparation 53

Compound (53) was obtained in a manner similar to Preparation 18 except that trifluoroacetic acid was used instead of 4N hydrogen chloride. The obtained compound was used in Preparation 93.

¹H-NMR (300 MHz, CDCl₃, δ): 0.62 (3H, t, J=7.3 Hz), 1.20 (3H, s), 1.49-2.15 (12H, m), 2.88-3.10 (2H, m), 3.34 (1H, m), 3.82 (1H, m), 4.07 (1H, m), 4.23-4.38 (3H, m), 4.92 (1H, m), 6.96-7.11 (2H, m), 7.14-7.28 (3H, m), 7.42 (2H, dd, J=7.6, 7.6 Hz), 7.50-7.58 (2H, m), 7.82 (2H, br), 8.01 (2H, d, J=7.6 Hz);

MASS (ES+): m/e 613.

Preparation 54

Compound (54) was obtained in a manner similar to Preparation 18. The obtained compound was used in Preparation 96.

Preparation 55

Compound (55) was obtained in a manner similar to Preparation 18. The obtained compound was used in Preparation 99.

Preparation 56

Compound (56) was obtained in a manner similar to Preparation 16.

Preparation 57

Compound (57) was obtained in a manner similar to Preparation 18 except that trifluoroacetic acid was used instead of 4N hydrogen chloride. The obtained compound was used in Preparation 102.

¹H-NMR (300 MHz, CDCl₃, δ): 0.70 (3H, t, J=7 Hz), 1.27 (3H, s), 1.49-2.10 (12H, m), 2.85-3.05 (3H, m), 3.70 (1H, m), 4.09 (1H, m), 4.24 (1H, m), 4.27-4.40 (2H, m), 4.83 (1H, m), 7.13-7.34 (5H, m), 7.42 (2×1H, dd, J=8.8 Hz), 7.55 (1H, m), 7.80 (2H, br), 7.89 (1H, s), 8.00 (2×1H, dd, J=8 and 1 Hz);

MASS (ES+): m/e 595.

Preparation 58

Compound (58) was obtained in a manner similar to Preparation 18. The obtained compound was used in Preparation 105.

Preparation 59

Compound (59) was obtained in a manner similar to Preparation 14.

Preparation 60

The Compound (59) (600 mg) was dissolved in dichloromethane (10 ml), tert-butoxycarbonyl-D-tert-leucine (444 mg), a solution of 1-ethyl-3-(3′-N,N-dimethylaminopropyl)carbodiimide (328 mg) in dichloromethane (2 ml) and hydroxybenzotriazole (285 mg) were added to the solution, and stirred for 15 hours at ambient temperature. The mixture was washed with 10% aqueous solution of citric acid (10 ml), water (20 ml), saturated aqueous sodium bicarbonate solution (20 ml) and saturated aqueous sodium chloride solution (20 ml), dried over sodium sulfate and the solvent was evaporated to give a crude compound as pale yellow oil. The crude compound was purified by flash column chromatography (Kieselgel 60, 30 g, eluent: ethyl acetate:hexane=1:2 to 1:1) to give Compound (60) (669 mg) as a white foam.

¹H-NMR (300 MHz, CDCl₃, δ): 0.60 (9×⅓H, s), 0.74 (9×⅔H, s), 1.36 (9×⅓H, s), 1.38 (9×⅔H, s), 1.64-2.30 (4H, m), 2.75-2.89 (1+⅓H, m), 2.93 (1×⅔H, dd, J=13.5, 6.5 Hz), 3.16-3.72 (2H, m), 3.84 (1×⅓H, d, J=10 Hz), 3.90 (1×⅔H, d, J=10 Hz), 4.17 (1×⅔H, dd, J=8, 4 Hz), 4.38 (1×⅓H, m), 4.80 (1×⅔H, m), 5.10 (1×⅓H, m), 6.40 (1×⅓H, d, J=10 Hz), 6.47 (1×⅔H, d, J=10 Hz), 7.12-7.30 (5H, m), 8.31 (1×⅔H, d, J=8 Hz), 8.65 (1×⅓H, d, J=8 Hz);

MASS (ES+): m/e 490.

Preparation 61

The Compound (61) (297 mg) was dissolved in dioxane (3 ml) and cold solution of 4N hydrogen chloride in dioxane (3 ml) was added to the mixture and stirred for 12 hours at ambient temperature. The mixture was evaporated to dryness to give Compound (61) (250 mg) as a white powder.

¹H-NMR (300 MHz, DMSO-d₆ δ): 0.63 (9×⅓H, s), 0.82 (9×⅔H, s), 1.60-2.30 (4H, m), 2.79-2.92 (1+⅓H, m), 2.97 ( [symbol] ⅔H, dd, J=13, 7 Hz), 3.05-3.66 (3H, m), 3.61 (3×⅔H, s), 3.75 (3×⅓H, s), 4.21 (1×⅔H, dd, J=8.5, 3.5 Hz), 4.55 (1×⅓H, m), 4.94 (1×⅔H, ddd, J=8, 8, 7 Hz), 5.14 (1×⅓H, dd, J=8, 4 Hz), 7.12-7.33 (5H, m), 8.10 (2H, br), 8.80 (1×⅔H, d, J=8 Hz), 9.03 (1×⅓H, d, J=8 Hz);

MASS (ES+): m/e 390.

Preparation 62

The Compound (61) (227 mg) was dissolved in dichloromethane (3 ml) and a solution of Compound (12) (217 mg) in dichloromethane (2 ml), hydroxybenzotriazole (86.4 mg) and a solution of 1-ethyl-3-(3′-N,N-dimethylaminopropyl)carbodiimide (99.3 mg) in dichloromethane (3 ml) were added to the solution, and the mixture was stirred for 15 hours at ambient temperature. The mixture was washed with 10% aqueous solution of citric acid (20 ml), saturated aqueous sodium bicarbonate solution (20 ml) and saturated aqueous sodium chloride solution (20 ml), dried over sodium sulfate and the solvent was evaporated to give a crude compound. The crude compound was purified by preparative thin layer chromatography (Merck Art 5717×2 plates, eluent: ethyl acetate:hexane=1:1) to give Compound (62) (297 mg) as a white foam.

¹H-NMR (300 MHz, DMSO-d₆ δ): 0.55 (9×⅓H, s), 0.70 (9×⅔H, s), 1.10-1.90 (10H, m), 1.22 (3H, d, J=7 Hz), 1.36 (9H, s), 1.93-2.33 (2H, m), 2.40-2.60 (2H, m), 2.71-2.89 (1+⅓H, m), 2.94 (1×⅔H, dd, J=13.7 Hz), 3.18-3.73 (2H, m), 3.53 (3×⅔H, s), 3.74 (3×⅓H, s), 3.95 (1H, m), 3.97 (1H, q, J=7 Hz), 4.16 (1×⅔H, dd, J=8.4 Hz), 4.23 (1×⅓H, d, J=10 Hz), 4.28 (1×⅔H, d, J=10 Hz), 4.45 (1H, d, J=12 Hz), 4.49 (1H, d, J=12 Hz), 4.82 (1H, m), 5.14 (1×⅓H, m), 6.91 (1×⅓H, m), 6.91 (1×⅓H, d, J=7 Hz), 6.95 (1×⅔H, d, J=7 Hz), 7.11-7.40 (10H, m), 7.49 (1×⅓H, d, J=10 Hz), 7.52 (1×⅔H, d, J=10 Hz), 8.50 (1×⅔H, d, J=8 Hz), 8.82 (1×⅓H, d, J=8 Hz);

MASS (ES−): m/e 777.

Preparation 63

Compound (63) was obtained in a manner similar to Preparation (17) except that 1N sodium hydroxide aqueous solution was used instead of the hydrogenation catalyst.

¹H-NMR (300 MHz, DMSO-d₆, δ): 0.52 (9× 5/9H, S), 0.72 (9× 4/9H; s), 1.10-1.90 (10H, m), 1.22 (3H, d, J=7 Hz), 1.35 (9× 5/9H, s), 1.37 (9× 4/9H, s), 2.15 (2H, m), 2.42-2.60 (2H, m), 2.70-3.00 (2H, m), 3.08-3.65 (2H, m), 3.95 (1H, m), 3.96 (1H, q, J=7 Hz), 4.10 (1× 4/9H, dd, J=8, 4 Hz), 4.24 (1× 5/9H, d, J=10 Hz), 4.27 (1× 4/9H, d, J=10 Hz), 4.38 (1× 4/9H, m), 4.45 (1H, d, J=12 Hz), 4.49 (1H, d, J=12 Hz), 4.83 (1× 5/9H, m), 5.02 (1× 5/9H, m), 6.91 (1× 5/9H, d, J=7.5 Hz), 6.95 (1× 4/9H, d, J=7.5 Hz), 7.10-7.40 (10H, m), 7.48 (1× 5/9H, brd, J=10 Hz), 7.51 (1× 4/9H, brd, J=19 Hz), 8.41 (1× 4/9H, d, J=8 Hz), 8.79 (1× 5/9H, d, J=8 Hz);

MASS (ES−): m/e 763.

Preparation 64

Compound (64) was obtained in a manner similar to Preparation (18). The obtained compound was used in Preparation 108.

¹H-NMR (300 MHz, DMSO-d₆, δ): 0.52 (9×½H, s), 0.73 (9×½H, s), 1.10-1.50 (4H, m), 1.21 (3×½H, d, J=6.5 Hz), 1.22 (3×½H, d, J=6.5 Hz), 1.58-1.96 (6H, m), 2.12-2.29 (2H, m), 2.35-2.60 (2H, m), 2.70-3.00 (2H, m), 3.06-3.66 (2H, m), 3.95 (1H, m), 3.96 (1×½H, q, J=6.5 Hz), 3.97 (1×½H, q, J=6.5 Hz), 4.10 (1×½H, m), 4.26-4.54 (3+½H, m), 4.85 (1×½H, m), 5.06 (1×½H, m), 7.14-7.41 (10H, m), 8.09 (2H, br), 8.55 (1×½H, d, J=8.5 Hz), 8.60 (1×½H, d, J=9 Hz), 8.67 (1×½H, d, J=8 Hz), 8.88 (1×½H, d, J=7 Hz);

MASS (ES−): m/e 663.

Preparation 65

2-Chlorotrityl chloride resin (Nova Biochem, 0.9 mmol C1/gram, 2.0 g) was washed with dichloromethane (3 ml) for 5 minutes twice. The resin was suspended in dichloromethane (3 ml) and to the suspension were added N-(9-fluorenylmethoxycarbonyl)-(R)-proline (1.82 g) in dichloromethane (3 ml) and N,N-diisopropylethylamine (698 mg). The suspension was shaken using rotary shaker for 15 minutes. Additionally, N,N-diisopropylethylamine (1.05 g) was added to the suspension and the mixture was shaken for 1 hour. The reagents and solvent were washed away and the residual solid was washed with dichloromethane (20 ml, 5 times), N,N-dimethylformamide (20 ml, 3 times), dichloromethane (20 ml, 3 times) and isopropyl alcohol (20 ml). The resulting solid was dried under vacuum to give Compound (65) (2.89 g).

To determine the loading value, the Compound (65) (300 mg) was treated with a mixture of dichloromethane-trifluoroacetic acid (1:1 v/v, 6 ml) for 1 hour. The Compound (65) was filtered and the filtrate was concentrated in vacuo to give 107 mg of N-(9-fluorenylmethoxycarbonyl)-(R)-proline (107 mg) which was identical with the starting material by HPLC analysis. Mightysil RP-18 GP 250-4.6 (5 mm) (Kanto Chemical Co., Ltd.), 0.1% TFA-acetonitrile/0.1% TFA-water 50:50 rt=12.15 minutes.

¹H-NMR (300 MHz, DMSO-d₆, δ): 1.78-2.34 (4H, m), 3.32-3.50 (2H, m), 4.11-4.37 (4H, m), 7.10-7.38 (3H, m), 7.43 (2H, t, J=7.7 Hz), 7.62-7.71 (2H, m), 7.90 (2H, dd, J=7.8, 4.1 Hz).

Preparation 66

A solution of piperidine in N,N-dimethylformamide (20% v/v, 20 ml) was added to the Compound B1-1 (2.00 g) and the resulting suspension was shaken using rotary shaker for 15 minutes. The suspension was filtered and then a solution of piperidine in N,N-dimethylformamide (20% v/v, 20 ml) was added to the residual solid. The suspension was shaken for additional 15 minutes. The suspension was filtered and the residual solid was washed with N,N-dimethylformamide (20 ml, 5 times). To the residual solid were added (S)-N-(9-fluorenylmethoxycarbonyl)phenylalanine (2.46 g), benzotriazole-1-yloxy-tris-pyrrolidinophosphonium hexafluorophosphate (PyBOP®; 3.31 g) and N,N-diisopropylethylamine (822 mg) at ambient temperature and the resulting suspension was shaken at the same temperature for 16 hours. The suspension was filtered and the residual solid was washed with N,N-dimethylformamide (20 ml, 5 times), dichloromethane (20 ml, 3 times) and isopropyl alcohol, and dried to give Compound (66) (2.08 g).

To determine the loading value, the Compound (66) (200 mg) was treated with a mixture of dichloromethane-trifluoroacetic acid (1:1 v/v, 4 ml) for 1 hour. The Compound (66) was filtered and the filtrate was concentrated in vacuo to give a dipeptide compound (79 mg). The purity of the dipeptide compound was determined by HPLC analysis. Mightysil RP-18 GP 250-4.6 (5 mm) (Kanto Chemical Co., Ltd.), 0.1% TFA-acetonitrile/0.1% TFA-water 50:50 rt=20.64 minutes.

¹H-NMR (300 MHz, CDCl₃, δ): 1.50-1.71 (2H, m), 1.74-1.91 (1H, m), 2.16-2, 34 (1H, m), 3.00 (1H, dd, J=12.5, 9.6 Hz), 3.12 (1H, dd, J=12.5, 5.7 Hz), 3.49-3.62 (1H, m), 4.21 (1H, t, J=6.6 Hz), 4.38 (2H, d, J=6.6 Hz), 4.65-4.80 (1H, m), 5.71 (1H, d, J=9.2 Hz), 7.12-7.46 (9H, m), 7.59 (2H, t, J=7.0 Hz), 7.77 (2H, d, J=7.4 Hz);

MASS (ES+): m/e 485.13 (M+1).

Preparation 67

A solution of piperidine in N,N-dimethylformamide (20% v/v, 20 ml) was added to the Compound (66) (1.6 g), and the resulting suspension was shaken using rotary shaker for 15 minutes. The suspension was filtered and then 20% N,N-dimethylformamide solution of piperidine (15 ml) was added to the residual solid and the suspension was shaken for additional 15 minutes. The suspension was filtered and the residual solid was washed with N,N-dimethylformamide (20 ml, 3 times). To the solid were added (S)-6-benzoyloxy-2-N-tert-butoxycarbonylaminohexanoic acid (1.53 g), benzotriazole-1-yloxy-tris-pyrrolidinephoponium hexafluorophosphate (PyBOP®; 2.34 g) and N,N-diisopropylethylamine (581 mg) at ambient temperature and the resulting suspension was shaken at the same temperature for 16 hours. The suspension was filtered and the residual solid was washed with N,N-dimethylformamide (10 ml, twice), isopropyl alcohol (10 ml), dichloromethane (10 ml, twice). This washing cycle was repeated once and then the solid was washed with isopropyl alcohol (10 ml) and diethyl ether (10 ml) successively, and dried to give Compound (67) (1.80 g).

To determine the loading value, the Compound (67) (300 mg) was treated with a mixture of dichloromethane-trifluoroacetic acid (1:1 v/v, 4 ml) for 1 hour. The Compound (67) was filtered and the filtrate was concentrated in vacuo and the residual solvent was removed azeotropically with toluene to give a tripeptide compound. The purity of the tripeptide compound was determined by HPLC analysis. Mightysil RP-18 GP 250-4.6 (5 mm) (Kanto chemical Co., Ltd.), 0.1% TFA-acetonitrile/0.1% TFA-water 40:60 rt=7.76 minutes.

¹H-NMR (300 MHz, CDCl₃, δ): 0.66-0.83 (3H, m), 1.19-2.38 (9H, m), 2.68-2.85 (1H, m), 2.91-3.12 (2H, m), 3.58-3.74 (1H, m), 4.11-4.25 (1H, m), 4.30-4.46 (3H, m), 4.98 (1H, brs), 5.71 (1H, brs), 7.11-7.52 (10H, m), 7.60 (2H, d, J=6.9 Hz), 7.76 (2H, d, J=7.3 Hz);

MASS (ES+): m/e 584.39 (M+i).

Preparation 68

A solution of piperidine in N,N-dimethylformamide (20% v/v, 100 ml) was added to the Compound (67) (1.15 g) and the suspension was shaken using rotary shaker for 15 minutes. The suspension was filtered, then a solution of piperidine in N,N-dimethylformamide (20% v/v, 100 ml) was added to the residual solid, and the suspension was shaken for additional 15 minutes. The suspension was filtered and washed with N,N-dimethylformamide (15 ml, 5 times). To the residual solid were added Compound (5) (1.15 g), benzotriazole-1-yloxy-tris-pyrrolidinephoponium hexafluorophosphate (PyBOP®; 1.69 g) and N,N-diisopropylethylamine (420 mg) at ambient temperature and the resulting suspension was shaken at the same temperature for 36 hours. The suspension was filtered and the residual solid was washed with N,N-dimethylformamide (10 ml, twice), isopropyl alcohol (10 ml), dichloromethane (10 ml, twice). This washing cycle was repeated and then the residual solid was washed with isopropyl alcohol (10 ml) and diethyl ether (20 ml) successively to give Compound (68) (300 mg).

Preparation 69

The Compound (68) (300 mg) was treated with a mixture of dichloromethane-trifluoroacetic acid (1:1 v/v, 6 ml) for 1 hour. The suspension was filtered and the filtrate was concentrated in vacuo to give Compound (69) (128 mg). The purity of the Compound B1-5 was determined by HPLC analysis. Mightysil RP-18 GP 250-4.6 (5 mm) (Kanto chemical Co., Ltd.), 0.1% TFA-acetonitrile/0.1% TFA-water 40:60 rt=7.76 minutes. The Compound (69) was used in Preparation 103.

¹H-NMR (300 MHz, CDCl₃, δ): 0.69 (3H, t, J=6.8 Hz), 1.28 (3H, s), 1.46-1.70 (3H, m), 1.71-2.08 (9H, m), 2.84-3.04 (3H, m), 3.63-3.78 (1H, m), 4.04-4.15 (1H, m), 4.20-4.38 (3H, m), 4.79-4.90 (1H, m), 7.11-7.32 (6H, m), 7.41 (2H, t, J=8.1 Hz), 7.45-7.62 (2H, m), 7.73-8.14 (5H, m);

MASS (ES+); m/e 595.21 (M+1).

Preparation 70

Compound (70) was obtained in a manner similar to Preparations 68.

Preparation 71

Compound (71) was obtained in a manner similar to Preparation 69. The obtained compound was used in Preparation 97.

¹H-NMR (300 MHz, CDCl₃, δ): 0.67 (3H, t, J=7.3 Hz), 1.29 (3H, s), 1.51-1.63 (2H, m), 1.63-2.06 (10H, m), 2.36 (3H, s), 2.83-3.0 (2H, m), 3.0-3.15 (1H, m), 3.68-3.78 (1H, m), 4.0-4.10 (1H, m), 4.26-4.40 (3H, m), 4.84 (1H, m), 5.20-5.45 (1H, brs), 7.10-7.32 (4H, m), 7.41 (2H, t, J=7.6 Hz), 7.52 (1H, t, J=7.3 Hz), 7.66 (1H, brd, J=3.3 Hz), 7.80-8.10 (1H, brs), 7.99 (2H, d, J=6.9 Hz).

Preparation 72

Compound (72) was obtained in a manner similar to Preparation 69. The obtained compound was used in Preparation 82.

¹H-NMR (300 MHz, CDCl₃, δ): 0.69 (3H, t, J=7.3 Hz), 1.32 (3H, s), 1.46-2.24 (12H, m), 2.81-3.11 (3H, m), 3.65-3.79 (1H, m), 3.97-4.58 (4H, m), 4.82-4.95 (1H, m), 6.95 (2H, t, J=8.8 Hz), 7.11-7.31 (4H, m), 7.36-7.82 (4H, m), 7.99 (2H, d, J=7.0 Hz), 8.04 (1H, brs);

MASS (ES+): m/e 613.21 (M+1, free).

Preparation 73

Compound (73) was obtained in a manner similar to Preparations 68. The obtained compound was used in Preparation 109.

¹H-NMR (300 MHz, CDCl₃, δ): 0.70 (3H, t, J=7.4 Hz), 1.29 (3H, s), 1.44-2.11 (12H, m), 2.80-3.03 (3H, m), 3.63-3.78 (1H, m), 3.76 (3H, s), 4.02-4.46 (4H, m), 4.75-4.88 (1H, m), 6.79 (2H, d, J=8.3 Hz), 7.09 (2H, d, J=8.3 Hz), 7.14-7.31 (2H, m), 7.36-7.80 (4H, m), 8.00 (2H, d, J=7.4 Hz), 8.13 (1H, brs);

MASS (ES+): m/e 625.28 (M+1, free).

Preparation 74

Compound (74) was obtained in a manner similar to Preparations 68. The obtained compound was used in Preparation 106.

¹H-NMR (300 MHz, CDCl₃, δ): 0.58-0.94 (6H, m), 0.95-1.33 (2H, m), 1.49-2.16 (16H, m), 3.00 (2H, brd, J=8.1 Hz), 3.03-3.18 (1H, m), 3.68-3.87 (1H, m), 4.02-4.16 (1H, m), 4.19-4.38 (3H, m), 4.67-4.83 (1H, m), 4.73-5.16 (2H, m), 7.11-7.35 (5H, m), 7.36-7.84 (4H, m), 7.94-8.19 (1H, brs), 7.97-8.04 (2H, m);

MASS (ES+): m/e 637.23 (M+1, free).

Preparation 75

Compound (75) was obtained in a manner similar to Preparation 68. The obtained compound was used in Preparation 100.

¹H-NMR (300 MHz, CDCl₃, δ): 0.48 (3H, t, J=7.3 Hz), 0.64 (3H, t, J=7.2 Hz), 0.72-0.91 (2H, m), 1.52-2.17 (12H, m), 2.91-3.11 (3H, m), 3.70-3.83 (1H, m), 3.97-4.43 (4H, m), 4.74-5.03 (1H, m), 7.13-7.34 (5H, m), 7.37-7.72 (4H, m), 7.76-7.84 (1H, m), 7.95-8.18 (2H, m), 7.97-8.04 (2H, m).

Preparation 76

To a stirred solution of benzotriazol-1-yl-oxy-tris-(N,N-dimethylamino)phosphoniumhexafluorophosphate (23.9 g) and 4-(N,N-dimethylamino)pyridine (7.6 g) in dry N,N-dimethylformamide (1.5 L), the Compound (18) (4.64 g) in dry N,N-dimethylformamide (8 ml) was added dropwise over 20 hours at room temperature. The volatiles were removed under reduced pressure and the residue was diluted with ethyl acetate (300 ml). The precipitate formed was collected by filtration, dissolved in ethyl acetate (50 ml), then washed with 5% aqueous potassium hydrogen sulfate solution (100 ml, 4 times), saturated aqueous sodium bicarbonate solution (100 ml, 3 times), water (100 ml) and brine (100 ml). The organic layer was dried over anhydrous magnesium sulfate and filtered. The filtrate was concentrated under reduced pressure and the residue was purified by flash column chromatography (eluting with ethyl acetate/hexane=1:1 v/v) to give Compound (76) (3.083 g) as a colorless amorphous.

¹H-NMR (300 MHz, CDCl₃, δ): 0.83 (3H, t, J=7.0 Hz), 1.28 (3H, s), 1.36-1.55 (2H, m), 1.59-1.99 (4H, m), 2.04-2.24 (2H, m), 2.24-2.40 (2H, m), 2.90 (1H, dd, J=13.6, 6.6 Hz), 3.19 (1H, dd, J=13.6, 9.9 Hz), 3.20-3.31 (1H, m), 3.80-3.91 (1H, m), 4.18-4.28 (1H, m), 4.32 (2H, t, J=6.2 Hz), 4.67 (1H, brd, J=5.5 Hz), 5.03 (2H, s), 5.14 (1H, dt, J=10 and 5.6 Hz), 5.85 (1H, s), 6.89 (2H, d, J=8.6 Hz), 7.14 (1H, s), 7.15 (2H, d, J=8.6 Hz), 7.28-7.48 (9H, m), 7.49-7.60 (2H, m), 8.00-8.06 (2H, m);

MASS (ES+): m/e 683.49 (M+1).

Preparation 77

To a stirred solution of the Compound (76) (3.07 g) in methanol (30 ml) was added 1N aqueous sodium hydroxide solution (11.2 ml, 2.5 eq) under ice-cooling and the mixture was stirred at ambient temperature for 4 hours. The pH of the mixture was adjusted to pH 7 with 1N hydrogen chloride, then methanol was evaporated under reduced pressure. The residue was extracted with ethyl acetate (300 ml). The organic layer was washed with saturated aqueous ammonium chloride (50 ml, twice), water (50 ml) and brine (50 ml), dried over sodium sulfate and filtered. The filtrate was concentrated in vacuo and the residue was purified by flash-column chromatography (ethyl acetate, then methanol/ethyl acetate=5:95 v/v) to give Compound (77) (2.63 g) as a colorless amorphous solid.

¹H-NMR (300 MHz, CDCl₃, δ): 0.84 (3H, t, J=7.4 Hz), 1.28 (3H, s), 1.20-1.92 (8H, m), 2.07-2.23 (2H, m), 2.24-2.39 (2H, m), 2.89 (1H, dd, J=13.8, 6.1 Hz), 3.18 (1H, dd, J=13.8, 9.5 Hz), 3.15-3.28 (1H, m), 3.65 (2H, d, J=6.5 Hz), 3.78-3.91 (1H, m), 4.15-4.28 (1H, m), 4.67 (1H, brd, J=5.8 Hz), 5.03 (2H, s), 5.13 (1H, dt, J=9.5, 6.2 Hz), 5.93 (1H, s), 6.88 (2H, d, J=8.5 Hz), 7.11-7.15 (1H, m), 7.14 (2H, d, J=8.5 Hz), 7.27-7.45 (5H, m), 7.52 (1H, d, J=10.2 Hz);

MASS (ES+): m/e 579.30 (M+1).

Preparation 78

To a stirred solution of the Compound (77) (1.0 g) in dichloromethane (50 ml) was added 1,1,1-triacetoxy-1,1-dihydro-1,2-benziodoxol-3(1H)-one (Dess-Martin periodinane) (3.66 g) in one portion under ice-cooling. The mixture was stirred at ambient temperature for 2 hours. The reaction was quenched with a solution of 20% sodium thiosulfate in saturated aqueous sodium bicarbonate solution (100 ml) under ice-cooling, then the mixture was extracted with ethyl acetate (100 ml), washed with saturated aqueous sodium bicarbonate solution, water and brine, dried over sodium sulfate, and evaporated in vacuo to give Compound (78) as a colorless amorphous (980 mg). The obtained compound was used in Example 1.

¹H-NMR (300 MHz, CDCl₃, δ): 0.84 (3H, t, J=7.3 Hz), 1.30 (3H, s), 1.50-1.91 (6H, m), 2.08-2.38 (4H, m), 2.46-2.55 (2H, brt, J=6.8 Hz), 2.90 (1H, dd, J=13.7, 5.9 Hz), 3.18 (1H, dd, J=13.7, 7.3 Hz), 3.20-3.30 (1H, m), 3.80-3.91 (1H, m), 4.17-4.29 (1H, m), 4.68 (1H, brd, J=6.3 Hz), 5.03 (2H, s), 5.14 (1H, dt, J=9.5, 5.6 Hz), 5.90 (1H, s), 6.89 (2H, d, J=8.5 Hz), 7.10-7.21 (1H, m), 7.14 (2H, d, J=8.5 Hz), 7.22-7.45 (5H, m), 7.47 (1H, d, J=10.3 Hz), 9.77 (1H, s);

MASS (ES+): m/e 577.25 (M+1).

Preparation 79

Compound (79) was obtained in a manner similar to Preparation 76.

¹H-NMR (300 MHz, CDCl₃, δ): 0.83 (3H, t, J=7.3 Hz), 1.28 (3H, s), 1.46 (2H, m), 1.60-1.98 (6H, m), 2.06-2.40 (4H, m), 2.90 (1H, dd, J=14, 6 Hz), 3.18 (1H, dd, J=14, 10 Hz), 3.26 (1H, m), 3.77 (3H, s), 3.86 (1H, m), 4.24 (1H, m), 4.32 (2H, t, J=6.5 Hz), 4.67 (1H, m), 5.14 (1H, ddd, J=10, 10, 6 Hz), 5.85 (1H, s), 6.81 (2×1H, d, J=9 Hz), 7.14 (2×1H, d, J=9 Hz), 7.14 (1H, d, J=10 Hz), 7.44 (2×1H, dd, J=7.5, 7.5 Hz), 7.50-7.60 (2H, m), 8.03 (2×1H, d, J=7.5 Hz);

MASS (ES−): m/e 605′.

Preparation 80

Compound (80) was obtained in a manner similar to Preparation 77.

¹H-NMR (300 MHz, CDCl₃, δ): 0.84 (3H, t, J=7.3 Hz), 1.25-1.51 (2H, m), 1.28 (3H, s), 1.54-1.94 (6H, m), 2.08-2.40 (4H, m), 2.89 (1H, dd, J=13.5, 6 Hz), 3.18 (1H, dd, J=13.5, 10 Hz), 3.25 (1H, m), 3.65 (2H, m), 3.77 (3H, s), 3.85 (1H, m), 4.22 (1H, dt, J=10 and 7.5 Hz), 4.67 (1H, m), 5.13 (1H, ddd, J=10, 10, 6 Hz), 5.99 (1H, s), 6.81 (2×1H, d, J=8.7 Hz), 7.14 (2×1H, d, J=8.7 Hz), 7.15 (1H, d, J=10 Hz), 7.53 (1H, d, J=10 Hz);

MASS (ES−): m/e 501.

Preparation 81

Compound (81) was obtained in a manner similar to Preparation 78. The obtained compound was used in Example 2.

¹H-NMR (300 MHz, CDCl₃, δ): 0.85 (3H, t, J=7.3 Hz), 1.29 (3H, s), 1.53-1.90 (6H, m), 2.08-2.37 (4H, m), 2.50 (2H, m), 2.89 (1H, dd, J=14, 6 Hz), 3.17 (1H, dd, J=14, 10 Hz), 3.25 (1H, m), 3.86 (1H, m), 4.23 (1H, m), 4.67 (1H, m), 5.13 (1H, ddd, J=10, 10, 6 Hz), 5.89 (1H, s), 6.81 (2×1H, d, J=8.8 Hz), 7.14 (2×1H, d, J=8.8 Hz), 7.16 (1H, d, J=11 Hz), 7.48 (1H, d, J=10 Hz), 9.77 (1H, t, J=1.4 Hz);

MASS (ES−): m/e 499.

Preparation 82

Compound (82) was obtained in a manner similar to Preparation 76.

¹H-NMR (300 MHz, CDCl₃, δ): 0.81 (3H, t, J=7.3 Hz), 1.27 (3H, s), 1.35-1.98 (8H, m), 2.06-2.40 (4H, m), 2.93 (1H, dd, J=13.6, 6.8 Hz), 3.20 (1H, dd, J=13.6, 9.6 Hz), 3.21-3.33 (1H, m), 3.78-3.90 (1H, m), 4.18-4.30 (1H, m), 4.32 (2H, t, J=6.4 Hz), 4.68 (1H, brd, J=7.7 Hz), 5.07-5.20 (1H, m), 5.84 (1H, S), 6.96 (2H, t, J=8.6 Hz), 7.10 (1H, d, J=10.3 Hz), 7.19 (1H, dd, J=8.6, 5.5 Hz), 7.44 (2H, t, J=7.3 Hz), 7.52-7.61 (2H, m), 8.03 (2H, d, J=8.4 Hz);

MASS (ES+): m/e 595.39 (M+1).

Preparation 83

Compound (83) was obtained in a manner similar to Preparation 77.

¹H-NMR (300 MHz, CDCl₃, δ): 0.84 (3H, t, J=7.3 Hz), 1.23-1.95 (8H, m), 1.29 (3H, S), 2.08-2.41 (4H, m), 2.94 (1H, dd, J=13.6, 6.2 Hz), 3.21 (1H, dd, J=13.6, 9.6 Hz), 3.23-3.33 (1H, m), 3.67 (2H, brt, J=6.2 Hz), 3.80-3.91 (1H, m), 4.16-4.30 (1H, m), 4.69 (1H brd, J=5.5 Hz), 5.07-5.20 (1H, m), 5.97 (1H, S), 6.97 (2H, t, J=8.5 Hz), 7.11 (1H, d, J=10.2 Hz), 7.20 (2H, dd, J=8.5, 5.1 Hz), 7.57 (1H, d, J=10.2 Hz);

MASS (ES+): m/e 491.45 (M+1).

Preparation 84

Compound (84) was obtained in a manner similar to Preparation 78. The obtained compound was used in Example 3.

¹H-NMR (300 MHz, CDCl₃, δ): 0.83 (3H, t, J=6.9 Hz), 1.29 (3H, S), 1.53-1.90 (6H, m), 2.08-2.38 (4H, m), 2.50 (2H, brt, J=7.0 Hz), 2.93 (1H, dd, J=13.9, 6.2 Hz), 3.19 (1H, dd, J=13.9, 9.1 Hz), 3.20-3.31 (1H, m), 3.79-3.90 (1H, m), 4.17-4.28 (1H, m), 4.68 (1H, brd, J=6.0 Hz), 5.07-5.19 (1H, m), 5.87 (1H, s), 6.96 (2H, t, J=8.9 Hz), 7.10 (1H, d, J=10.1 Hz), 7.19 (2H, dd, J=8.9, 5.5 Hz), 7.50 (1H, d, J=10.3 Hz), 9.77 (1H, s);

MASS (ES+): m/e 489.42 (M+1).

Preparation 85

Compound (85) was obtained in a manner similar to Preparation 76.

¹H-NMR (300 MHz, CDCl₃, δ): 1.31-1.96 (14H, m), 2.08-2.23 (1H, m), 2.24-2.37 (2H, m), 2.43-2.56 (2H, m), 2.95 (1H, dd, J=13.5, 5.7 Hz), 3.14-3.28 (1H, m), 3.26 (1H, dd, J=13.5, 10.5 Hz), 3.84-3.95 (1H, m), 4.23 (1H, dt, J=10.2, 7.8 Hz), 4.31 (2H, t, J=6.6 Hz), 4.63-4.69 (1H, m), 5.15 (1H, ddd, J=10.2, 10.2, 6.0 Hz), 6.13 (1H, s), 7.16-7.31 (6H, m), 7.39-7.48 (3H, m), 7.52-7.60 (1H, m), 8.00-8.05 (2H, m);

MASS (ES+): m/e 589.40 (M+1).

Preparation 86

Compound (86) was obtained in a manner similar to Preparation 77.

¹H-NMR (300 MHz, CDCl₃, δ): 1.20-1.81 (14H, m), 2.10-2.22 (1H, m), 2.25-2.37 (2H, m), 2.43-2.58 (2H, m), 2.95 (1H, dd, J=13.5, 5.7 Hz), 3.13-3.28 (1H, m), 3.25 (1H, dd, J=13.5, 10.2 Hz), 3.65 (2H, t, J=6.3 Hz), 3.85-3.95 (1H, m), 4.22 (1H, dt, J=10.2, 7.2 Hz), 4.67 (1H, dd, J=7.8, 2.1 Hz), 5.15 (1H, ddd, J=10.2, 10.2, 6.0 Hz), 6.28 (1H, s), 7.16-7.31 (6H, m), 7.44 (1H, d, J=10.2 Hz);

MASS (ES+): m/e 485.39 (M+1).

Preparation 87;

Compound (87) was obtained in a manner similar to Preparation 78. The obtained compound was used in Example 4.

¹H-NMR (300 MHz, CDCl₃, δ): 1.42-1.92 (13H, m), 2.08-2.22 (1H, m), 2.23-2.37 (2H, m), 2.42-2.56 (2H, m), 2.95 (1H, dd, J=13.8, 5.7 Hz), 3.13-3.28 (1H, m), 3.25 (1H, dd, J=13.8, 10.2 Hz), 3.85-3.95 (1H, m), 4.22 (1H, dt, J=10.2, 7.2 Hz), 4.64-4.69 (1H, m), 5.15 (1H, ddd, J=9.9, 9.9, 5.7 Hz), 6.15 (1H, s), 7.17-7.31 (6H, m), 7.44 (1H, d, J=10.2 Hz), 9.77 (1H, s);

MASS (ES+): m/e 483.36 (M+1).

Preparation 88

Compound (88) was obtained in a manner similar to Preparation 76.

¹H-NMR (300 MHz, CDCl₃, δ): 0.790 (3H, t, J=7.2 Hz), 1.27 (3H, s), 1.38-1.98 (8H, m), 2.07-2.38 (4H, m), 3.06 (1H, dd, J=14.1, 6.9 Hz), 3.28-3.36 (1H, m), 3.26 (1H, dd, J=14.1, 8.4 Hz), 3.79-3.89 (1H, m), 4.25 (1H, dt, J=10.2, 7.8 Hz), 4.32 (2H, t, J=6.3 Hz), 4.65-4.71 (1H, m), 5.17 (1H, dt, J=9.0, 6.9 Hz), 5.89 (1H, s), 7.01 (1H, d, J=10.2 Hz), 7.32-7.38 (2H, m), 7.40-7.48 (2H, m), 7.52-7.63 (3H, m), 7.61-7.67 (1H, m), 8.00-8.06 (2H, m);

MASS (ES+): m/e 602.47 (M+1).

Preparation 89

Compound (89) was obtained in a manner similar to Preparation 77.

¹H-NMR (300 MHz, CDCl₃, δ): 0.809 (3H, t, J=7.2 Hz), 1.24-1.94 (9H, m), 1.28 (3H, s), 2.06-2.41 (4H, m), 3.06 (1H, dd, J=9.0, 6.9 Hz), 3.26-3.36 (1H, m), 3.26 (1H, dd, J=13.5, 9.0 Hz), 3.66 (2H, t, J=6.3 Hz), 3.79-3.90 (1H, m), 4.24 (1H, dt, J=10.2, 7.8 Hz), 4.65-4.72 (1H, m), 5.18 (1H, dt, J=9.0, 7.2 Hz), 6.01 (1H, s), 7.02 (1H, d, J=10.2 Hz), 7.35 (2H, d, J=8.1 Hz), 7.58 (2H, d, J=8.1 Hz), 7.64 (1H, d, J=10.2 Hz);

MASS (ES+): m/e 498.41 (M+1).

Preparation 90

Compound (90) was obtained in a manner similar to Preparation 78. The obtained compound was used in Example 5.

¹H-NMR (300 MHz, CDCl₃, δ): 0.812 (3H, t, J=7.2 Hz), 1.29 (3H, s), 1.49-1.92 (6H, m), 2.07-2.40 (4H, m), 2.51 (2H, t, J=7.2 Hz), 3.06 (1H, dd, J=13.5, 6.9 Hz), 3.26-3.36 (1H, m), 3.26 (1H, dd, J=13.5, 8.7 Hz), 3.78-3.90 (1H, m), 4.24 (1H, dt, J=10.2, 7.2 Hz), 4.65-4.71 (1H, m), 5.18 (1H, dt, J=9.0, 8.4 Hz), 5.93 (1H, s), 7.02 (1H, d, J=10.2 Hz), 7.35 (2H, d, J=8.7 Hz), 7.57-7.59 (1H, m), 7.58 (2H, d, J=8.8 Hz), 9.77 (1H, s);

MASS (ES+): m/e 496.46 (M+1).

Preparation 91

Compound (91) was obtained in a manner similar to Preparation 76.

¹H-NMR (300 MHz, CDCl₃, δ): 0.82 (3H, t, J=7.5 Hz), 1.27 (3H, s), 1.39 (3H, t, J=7.2 Hz), 1.40-1.52 (2H, m), 1.64-1.98 (6H, m), 2.06-2.39 (4H, m), 2.88 (1H, dd, J=13.5, 5.7 Hz), 3.09-3.32 (2H, m), 3.79-3.90 (1H, m), 3.99 (2H, q, J=7.2 Hz), 4.18-4.30 (1H, m), 4.31 (2H, t, J=6.0 Hz), 4.62-4.69 (1H, m), 5.07-5.18 (1H, dt, J=9.9, 6.0 Hz), 5.82 (1H, s), 6.79 (2H, d, J=8.4 Hz), 7.10-7.18 (1H, m), 7.13 (2H, d, J=8.4 Hz), 7.38-7.59 (4H, m), 7.99-8.05 (2H, m);

MASS (ES+): m/e 621.55 (M+1).

Preparation 92

Compound (92) was obtained in a manner similar to Preparation 77.

¹H-NMR (300 MHz, CDCl₃, δ): 0.84 (3H, t, J=7.2 Hz), 1.28-1.93 (8H, m), 1.28 (3H, s), 1.39 (3H, t, J=6.9 Hz), 2.08-2.23 (2H, m), 2.24-2.39 (2H, m), 2.88 (1H, dd, J=13.5, 6.0 Hz), 3.17 (1H, dd, J=13.5, 9.9 Hz), 3.20-3.30 (1H, m), 3.65 (2H, t, J=6.6 Hz), 3.80-3.90 (1H, m), 3.99 (2H, q, J=6.9 Hz), 4.22 (1H, dt, J=10.2, 7.8 Hz), 4.64-4.69 (1H, m), 5.13 (1H, dt, J=9.9, 6.0 Hz), 5.93 (1H, s), 6.79 (2H, d, J=8.4 Hz), 7.10-7.17 (1H, m), 7.13 (2H, d, J=8.4 Hz), 7.52 (1H, d, J=10.2 Hz);

MASS (ES+): m/e 517.44 (M+1).

Preparation 93

Compound (93) was obtained in a manner similar to Preparation 78. The obtained compound was used in Example 6.

¹H-NMR (300 MHz, CDCl₃, δ): 0.85 (3H, t, J=7.2 Hz), 1.29 (3H, s), 1.40 (3H, t, J=6.9 Hz), 1.49-1.92 (6H, m), 2.09-2.24 (2H, m), 2.24-2.39 (2H, m), 2.50 (2H, dt, J=6.3, 1.2 Hz), 2.88 (1H, dd, J=14.1, 5.7 Hz), 3.17 (1H, dd, J=14.1, 10.2 Hz), 3.20-3.30 (1H, m), 3.81-3.90 (1H, m), 3.99 (2H, q, J=6.9 Hz), 4.23 (1H, dt, J=10.2, 7.2 Hz), 4.64-4.70 (1H, m), 5.13 (1H, dt, J=10.2, 5.7 Hz), 5.85 (1H, s), 6.80 (2H, d, J=8.4 Hz), 7.12-7.19 (1H, m), 7.13 (2H, d, J=8.4 Hz), 7.46 (1H, d, J=10.2 Hz), 9.77 (1H, t, J=1.2 Hz);

MASS (ES+): m/e 515.36 (M+1).

Preparation 94

Compound (94) was obtained in a manner similar to Preparation 76.

¹H-NMR (300 MHz, CDCl₃, δ): 0.78 (3H, t, J=7.5 Hz), 1.25 (3H, s), 1.46 (2H, m), 1.58-1.95 (6H, m), 2.07-2.39 (4H, m), 3.11 (1H, dd, J=14, 8 Hz), 3.16 (1H, dd, J=14, 8 Hz), 3.41 (1H, m), 3.88 (1H, m), 4.24 (1H, m), 4.32 (2H, t, J=6.5 Hz), 4.70 (1H, dd, J=8, 3 Hz), 5.24 (1H, ddd, J=10, 8, 8 Hz), 5.80 (1H, s), 6.97-7.31 (5H, m), 7.44 (2H, dd, J=7.5, 7.5 Hz), 7.50-7.60 (2H, m), 8.00-8.06 (2H, m);

MASS (ES+): m/e 595;

MASS (ES−): m/e 593.

Preparation 95

Compound (95) was obtained in a manner similar to Preparation 77.

¹H-NM (300 MHz, CDCl₃, δ): 0.79 (3H, t, J=7.5 Hz), 1.22-1.51 (2H, m), 1.26 (3H, s), 1.52-1.73 (3H, m), 1.74-1.94 (3H, m), 2.08-2.40 (4H, m), 3.10 (1H, dd, J=15, 8 Hz), 3.15 (1H, dd, J=15, 8 Hz), 3.41 (1H, m), 3.66 (2H, t, J=7 Hz), 3.88 (1H, m), 4.23 (1H, m), 4.70 (1H, m), 5.24 (1H, ddd, J=10, 8, 8 Hz), 5.91 (1H, s), 6.97-7.08 (2H, m), 7.10 (1H, d, J=10 Hz), 7.15-7.28 (2H, m), 7.54 (1H, d, J=10 Hz);

MASS (ES+): m/e 491;

MASS (ES−): m/e 489.

Preparation 96

Compound (96) was obtained in a manner similar to Preparation 78. The obtained compound was used in Example 7.

¹H-NMR (300 MHz, CDCl₃, δ): 0.80 (3H, t, J=7 Hz), 1.26 (3H, s), 1.50-1.94 (6H, m), 2.11-2.44 (4H, m), 2.51 (2H, m), 3.05-3.20 (2H, m), 3.41 (1H, m), 3.89 (1H, m), 4.24 (1H, m), 4.71 (1H, m), 5.24 (1H, m), 5.85 (1H, s), 6.97-7.28 (5H, m), 7.49 (1H, d, J=10 Hz), 9.78 (1H, s);

MASS (ES+): m/e 489;

MASS (ES−): m/e 487.

Preparation 97

Compound (97) was obtained in a manner similar to Preparation 76.

¹H-NMR (300 MHz, CDCl₃, δ): 0.83 (3H, t, J=7.3 Hz), 1.28 (3H, s), 1.25-1.47 (2H, m), 1.56-1.74 (4H, m), 1.76-1.89 (2H, m), 2.15-2.36 (4H, m), 2.93 (1H, dd, J=13.6, 6.6 Hz), 3.20 (1H, dd, J=13.6, 9.5 Hz), 3.20-3.32 (1H, m), 3.66 (2H, t, J=6.6 Hz), 3.85 (1H, ddd, J=13.2, 8.1, 4.4 Hz), 4.22 (1H, ddd, J=15, 7.6, 2.2 Hz), 4.67 (1H, brd, J=5.8 Hz), 5.15 (1H, ddd, J=16.5, 9.5, 6.6 Hz), 5.99 (1H, s), 7.08° (1H, d, J=10.6 Hz), 7.16 (2H, d, J=8.9 Hz), 7.22 (2H, d, J=8.9 Hz), 7.58 (1H, d, J=10.3 Hz).

Preparation 98

Compound (98) was obtained in a manner similar to Preparation 77.

¹H-NMR (300 MHz, CDCl₃, δ): 0.81 (3H, t, J=7.3 Hz), 1.27 (3H, s), 1.41-1.58 (2H, m), 1.61 (3H, s), 1.71-1.90 (4H, m), 2.05-2.34 (4H, m), 2.95 (1H, dd, J=13.5, 6.2 Hz), 3.20 (1H, dd, J=13.5, 9.2 Hz), 3.25-3.36 (1H, m), 3.82-3.89 (1H, m), 4.25 (1H, dd, J=17.9, 10.2 Hz), 4.32 (2H, t, J=6.2 Hz), 4.68 (1H, brd, J=6.6 Hz), 5.14 (1H, ddd, J=16.7, 9.5, 6.6 Hz), 5.81 (1H, s), 7.08 (1H, d, J=9.9 Hz), 7.16 (2H, d, J=8.1 Hz), 7.24 (2H, d, J=8.1 Hz), 7.44 (2H, t, J=8.4 Hz), 7.56 (1H, dd, J=6.6, 4.3 Hz), 8.03 (2H, d, J=7.3 Hz).

Preparation 99

Compound (99) was obtained in a manner similar to Preparation 78. The obtained compound was used in Example 8.

¹H-NMR (300 MHz, CDCl₃, δ): 0.83 (3H, t, J=7.5 Hz), 1.29 (3H, s), 1.52-1.90 (6H, m), 2.08-2.38 (4H, m), 2.50 (2H, m), 2.94 (1H, dd, J=13.5, 6 Hz), 3.19 (1H, dd, J=13.5, 9.5 Hz), 3.28 (1H, m), 3.85 (1H, m), 4.24 (1H, m), 4.68 (1H, m), 5.14 (1H, ddd, J=10, 9.5, 6 Hz), 5.89 (1H, s), 7.09 (1H, d, J=10.5 Hz), 7.16 (2×1H, d, J=8.5 Hz), 7.25 (2×1H, d, J=8.5 Hz), 7.52 (1H, d, J=10 Hz), 9.77 (1H, t, J=1.3 Hz);

MASS (ES−): m/e 503.

Preparation 100

Compound (100) was obtained in a manner similar to Preparation 76.

¹H-NMR (300 MHz, CDCl₃, δ): 0.75 (3H, t, J=7.3 Hz), 0.91 (3H, t, J=7.3 Hz), 1.35-1.98 (10H, m), 2.10-2.43 (4H, m), 2.97 (1H, dd, J=13.5, 6.4 Hz), 3.24 (1H, dd, J=13.5, 9.4 Hz), 3.21-3.30 (1H, m), 3.83-3.94 (1H, m), 4.25 (1H, dt, J=10.3, 7.6 Hz), 4.32 (2H, t, J=6.2 Hz), 4.63-4.70 (1H, m), 5.18 (1H, dt, J=10.2, 6.3 Hz), 5.78 (1H, s), 7.13 (1H, d, J=10.3 Hz), 7.19-7.32 (5H, m), 7.40-7.47 (2H, m), 7.50 (1H, d, J=10.2 Hz), 7.51-7.60 (1H, m), 8.01-8.06 (2H, m);

MASS (ES+): m/e 591.21 (M+1).

Preparation 101

Compound (101) was obtained in a manner similar to Preparation 77.

¹H-NMR (300 MHz, CDCl₃, δ): 0.77 (3H, t, J=7.7 Hz), 0.91 (3H, t, J=7.3 Hz), 1.20-1.93 (10H, m), 2.07-2.45 (4H, m), 2.97 (1H, dd, J=13.5, 6.2 Hz), 3.24 (1H, dd, J=13.5, 9.1 Hz), 3.21-3.30 (1H, m), 3.66 (2H, t, J=6.6 Hz), 3.82-3.93 (1H, m), 4.24 (1H, dd, J=10.0, 7.2 Hz), 4.67 (1H, brd, J=8.0 Hz), 5.12-5.23 (1H, m), 5.84 (1H, s), 7.12 (1H, d, J=10.0 Hz), 17.16-7.31 (5H, m), 7.49 (1H, d, J=10.4 Hz);

MASS (ES+): m/e 487.19 (M+1).

Preparation 102

Compound (102) was obtained in a manner similar to preparation 78. The obtained compound was used in Example 9.

¹H-NMR (300 MHz, CDCl₃, δ): 0.77 (3H, t, J=7.4 Hz), 0.91 (3H, t, J=7.4 Hz), 1.50-1.92 (8H, m), 2.07-2.42 (4H, m), 2.51 (2H, brt, J=6.1 Hz), 2.96 (1H, dd, J=13.1 and 5.7 Hz), 3.17-3.30 (2H, m), 3.83-3.94 (1H, m), 4.18-4.30 (1H, m), 4.67 (1H, brd, J=6.1 Hz), 5.12-5.23 (1H, m), 5.85 (1H, s), 7.15 (1H, d, J=10.8 Hz), 7.18-7.31 (5H, m), 7.44 (1H, d, J=10.0 Hz), 9.77 (1H, s);

MASS (ES+): m/e 485.29 (M+1).

Preparation 103

Compound (103) was obtained in a manner similar to Preparation 76 except that benzotriazol-1-yloxy-tris-pyrrolidinephophonium hexafluorophosphate was used instead of benzotriazol-1-yloxy-tris-(dimethylamino)phosphonium hexafluorophosphate.

¹H-NMR (300 MHz, CDCl₃, δ): 0.81 (3H, t, J=7.3 Hz), 1.28 (3H, s), 1.46 (2H, m), 1.61-2.00 (6H, m), 2.06-2.39 (4H, m), 2.97 (1H, dd, J=13.5, 6 Hz), 3.24 (1H, dd, J=13.5, 9.5 Hz), 3.26 (1H, m), 3.86 (1H, m), 4.24 (1H, m), 4.32 (2H, t, J=6.5 Hz), 4.67 (1H, m), 5.18 (1H, m), 5.82 (1H, s), 7.13 (1H, d, J=11 Hz), 7.16-7.32 (5H, m), 7.39-7.59 (2H, m), 7.51-7.60 (2H, m), 7.95-8.08 (2H, m);

MASS (ES−): m/e 575.

Preparation 104

Compound (104) was obtained in a manner similar to Preparation 77.

¹H-NMR (300 MHz, CDCl₃, δ): 0.83 (3H, t, J=7.5 Hz), 1.22-1.95 (8H, m), 1.28 (3H, s), 2.07-2.40 (4H, m), 2.96 (1H, dd, J=13, 6.5 Hz), 3.04 (1H, dd, J=13, 9 Hz), 3.06 (1H, m), 3.65 (2H, brt, J=6 Hz), 3.86 (1H, m), 4.23 (1H, m), 4.68 (1H, m), 5.19 (1H, ddd, J=10, 9, 6 Hz), 5.93 (1H, s), 7.12 (1H, d, J=11 Hz), 7.16-7.32 (5H, m), 7.55 (1H, d, J=10 Hz);

MASS (ES−): m/e 471.

Preparation 105

Compound (105) was obtained in a manner similar to Preparation 78. The obtained compound was used in Example 10.

¹H-NMR (300 MHz, CDCl₃, δ): 0.84 (3H, t, J=7.3 Hz), 1.29 (3H, s), 1.48-1.95 (6H, m), 2.06-2.59 (6H, m), 2.97 (1H, dd, J=13.5, 6 Hz), 3.24 (1H, dd, J=13.5, 10 Hz), 3.26 (1H, m), 3.86 (1H, m), 4.24 (1H, m), 4.68 (1H, dd, J=8, 2 Hz), 5.19 (1H, ddd, J=10, 10, 6 Hz), 5.92 (1H, s), 7.16 (1H, d, J=11 Hz), 7.16-7.33 (5H, m), 7.50 (1H, d, J=10 Hz), 9.77 (1H, brs);

MASS (ES−): m/e 469.

Preparation 106

Compound (106) was obtained in a manner similar to Preparation 76.

¹H-NMR (300 MHz, CDCl₃, δ): 0.87 (3H, t, J=7.2 Hz), 0.96 (3H, t, J=7.0 Hz), 0.93-1.04 (1H, m), 1.11-1.36 (3H, m), 1.37-1.64 (3H, m), 1.65-1.96 (7H, m), 2.00-2.24 (2H, m), 2.27-2.42 (2H, m), 2.98 (1H, dd, J=13.6, 6.6 Hz), 3.21-3.32 (1H, m), 3.23 (1H, dd, J=13.6, 9.5 Hz), 3.81-3.93 (1H, m), 4.18-4.29 (1H, m), 4.32 (2H, t, J=6.5 Hz), 4.67 (1H, brd, J=7.7 Hz), 5.10-5.23 (1H, m), 5.78 (1H, s), 7.13 (1H, d, J=10.2 Hz), 7.19-7.32 (5H, m), 7.40-7.60 (4H, m), 8.01-8.06 (2H, m);

MASS (ES+): m/e 619.34 (M+1).

Preparation 107

Compound (107) was obtained in a manner similar to Preparation 77.

¹H-NMR (300 MHz, CDCl₃, δ): 0.89 (3H, t, J=7.0 Hz), 0.96 (3H, t, J=6.8 Hz), 0.97-1.08 (1H, m), 1.12-1.92 (13H, m), 2.02-2.26 (2H, m), 2.27-2.44 (2H, m), 2.98 (1H, dd, J=13.5, 6.6 Hz), 3.20-3.31 (1H, m), 3.22 (1H, dd, J=13.5, 9.6 Hz), 3.66 (2H, brt, J=6.3 Hz), 3.82-3.92 (1H, m), 4.22 (1H, dt, J=10.2, 7.6 Hz), 4.67 (1H, brd, J=7.5 Hz), 5.11-5.22 (1H, m), 5.86 (1H, s), 7.12 (1H, d, J=10.3 Hz), 7.17-7.31 (5H, m), 7.49 (1H, d, J=10.3 Hz);

MASS (ES+): m/e 515.23 (M+1).

Preparation 108

Compound (108) was obtained in a manner similar to Preparation 78.

¹H-NMR (300 MHz, CDCl₃, δ): 0.89 (3H, t, J=6.6 Hz), 0.94-1.08 (1H, m), 0.96 (3H, t, J=6.9 Hz), 1.10-1.38 (4H, m), 1.43-1.92 (6H, m), 2.00-2.42 (5H, m), 2.50 (2H, brt, J=6.6 Hz), 2.98 (1H, dd J=13.5, 6.6 Hz), 3.20-3.31 (1H, m), 3.22 (1H, dd, J=13.5, 9.2 Hz), 3.81-3.92 (1H, m), 4.16-4.28 (1H, m), 4.67 (1H, J=5.8 Hz), 5.10-5.22 (1H, m), 5.84 (1H, s), 7.14 (1H, d, J=10.3 Hz), 7.15-7.32 (5H, m), 7.43 (1H, d, J=10.2 Hz), 9.77 (1H, brs);

MASS (ES+): m/e 513.26 (M+1).

Preparation 109

Compound (109) was obtained in a manner similar to Preparation 76.

¹H-NMR (300 MHz, CDCl₃, δ): 1.04 (3×3H, S), 1.26-1.40 (4H, m), 1.33 (3H, d, J=7 Hz), 1.48-1.92 (6H, m), 2.16 (1H, m), 2.34 (1H, m), 2.54 (2H, m), 2.90 (1H, dd, J=13, 5 Hz), 3.02 (1H, m), 3.18 (1H, dd, J=13, 10 Hz), 3.90 (1H, m), 3.92 (1H, q, J=7 Hz), 4.32 (1H, dt, J=10, 7.5 Hz), 4.49 (1H, d, J=12 Hz), 4.55 (1H, d, J=12 Hz), 4.59 (1H, m), 5.01 (1H, ddd, J=10, 10, 5 Hz), 6.21 (1H, d, J=10 Hz), 6.23 (1H, d, J=10 Hz), 7.13 (1H, d, J=10 Hz), 7.16-7.41 (10H, m);

MASS (ES+): m/e 647.

Preparation 110

To a stirred solution of 2-indanone (12.5 g) in a mixture of ethanol (210 ml) and water (210 ml) was added sodium cyanide (11.6 g) and ammonium carbonate (100 g) at ambient temperature. The mixture was heated at 55 to 60° C. for 6 hours and then allowed to cool to ambient temperature. The mixture was stirred at 0° C. for half an hour and the precipitated solid was collected. The solid was recrystallized from ethanol to give 2-spirohydantoinindane (4.5 g).

¹H-NMR (300 MHz, DMSO-d₆, δ): 3.04 (1H, s), 3.10 (1H, s), 3.22-3.42 (1H, br), 3.33 (1H, s), 3.38 (1H, s), 7.15-7.27 (4H, m), 10.25 (1H, brs);

MASS (ES+): m/e 202.18 (M).

Preparation 111

To a stirred solution of 2-spirohydantoinindane in propylene glycol (13 ml) was added 40% aqueous solution of sodium hydoxide (22 ml) at ambient temperature. The mixture was refluxed for 24 hours. The reaction mixture was allowed to cool and then diluted with water (50 ml). After acidification with 1 N hydrochloric acid to pH 2, the precipitated solid was filtered and the filtrate was neutralized by addition of a 10% (w/v) aqueous sodium bicarbonate solution. The mixture was stirred for an hour and left overnight at 0° C. Most of the solvent was removed under reduced pressure and the residue was stirred at 0° C. The precipitate were collected by filtration and recrystallyzed from ethanol/water to give 2-aminoindan-2-carboxylic acid (2.76 g) as a white-scaled crystal.

¹H-NMR (300 MHz, D₂O, δ): 3.23 (1H, s), 3.29 (1H, s), 3.64 (1H, s), 3.70 (1H, s), 7.28-7.38 (4H, m);

MASS (ES+): m/e 178.00 (M+1).

Preparation 112

To a stirred solution of methyl (2R)-2-hydroxypropanoate (25 g) in N,N-dimethylformamide (250 ml) was added imidazole (66 g) followed by tert-butyldiphenylchlorosilane (68.08 g) at 0° C. The mixture was stirred at ambient temperature for two hours. The reaction mixture was poured into water and extracted with ethyl acetate. The organic layer was successively washed with water, 0.2 N hydrochloric acid, saturated aqueous sodium bicarbonate solution and brine. The organic layer was dried over magnesium sulfate, filtered and evaporated to give methyl (2R)-2-tert-butyldiphenylsilylpropanoate (80.5 g) as a colorless oil.

¹H-NMR (300 MHz, CDCl₃, δ): 1.09 (9H, s), 1.37 (3H, d, J=6.9 Hz), 3.56 (3H, s), 4.27 (1H, q, J=6.9 Hz), 7.32-7.48 (6H, m), 7.63-7.75 (4H, m);

MASS (ES+): m/e 375.29 (M+Na).

Preparation 113

To a stirred solution of dimethyl methylphosphonate (145 g) in tetrahydrofuran (750 ml) was added n-butyllithium (1.6 M in hexane, 127 ml) dropwise at −78° C. over an hour and the resulting mixture was stirred at the same temperature for an hour. To this mixture was added dropwise a solution of methyl-(2R)-2-tert-butyldiphenylsilyloxypropanoate in tetrahydrofuran (450 ml) over an hour. The mixture was stirred at the same temperature for two hours, allowed to warm to −30° C. over an hour and stirred at ambient temperature for half an hour. The reaction mixture was poured into a stirred saturated ammonium chloride (2000 ml) in an ice bath and left at ambient temperature overnight. The aqueous phase was separated and extracted with ethyl acetate twice. The combined organic extracts were washed with water and brine, and dried over magnesium sulfate. The organic layer was filtered and concentrated in vacuo. The crude product was purified by flash chromatography eluting with 33 to 60% ethyl acetate/hexane (v/v) to give dimethyl-(3R)-3-tert-buthyldiphenylsilyloxy-2-oxobutylphosphate (81.1 g) as a colorless oil.

¹H-NMR (300 MHz, CDCl₃, δ): 1.10 (9H, s), 2.21 (3H, d, J=6.9 Hz), 3.08 (1H, dd, J=21.9, 15.0 Hz), 3.48 (1H, dd, J=20.4, 15.0 Hz), 3.73 (3H, s), 3.77 (3H, s), 4.25 (3H, q, J=6.9 Hz), 7.33-7.49 (6H, m), 7.58-7.68 (4H, m);

MASS (ES+): m/e 435.31 (M+1).

Preparation 114

To a stirred solution of crude (2R)-2-aminobutanoic acid (12.1 g) in aqueous sulfuric acid (0.88 M, 200 ml) was added an aqueous sodium nitrite (8.8 M, 20 ml) dropwise at 0° C. over two hours. The mixture was left at the same temperature and allowed to warm to ambient temperature. Additional concentrated sulfuric acid (10 ml) and aqueous sodium nitrite (12.1 g) were added at 0° C. after thirteen hours and the mixture was left at ambient temperature for two days. Half of the volume of the solvent was evaporated under reduced pressure and the resulting solution was adjusted to pH 2 with saturated aqueous sodium bicarbonate solution. The resulting mixture was extracted twice with ethyl acetate. The combined organic extracts were washed with brine, dried over magnesium sulfate, filtered and evaporated carefully to give crude (2R)-2-hydroxybutanoic acid (6.57 g), which was used directly for the next step without further purification.

¹H-NMR (300 MHz, CDCl₃, δ): 1.03 (3H, t, J=7.5 Hz), 0.77 (1H, m), 1.90 (1H, m), 4.26 (1H, t, J=5 Hz);

MASS (ES−): m/e 103.

Preparation 115

To a stirred solution of crude (2R)-2-hydroxybutanoic acid (2.0 g) in a mixture of methanol (5 ml) and ether (15 ml) was added (trimethylsilyl)diazomethane (2.0 M in hexane, 9.6 ml) dropwise in an ice bath. The reaction mixture was stirred at ambient temperature overnight. The solvent was evaporated carefully to give crude methyl (2R)-2-hydroxybutanoate as a pale yellow oil (1.9 g), which was used directly for the next step without further purification.

¹H-NMR (300 MHz, CDCl₃, δ): 0.96 (3H, t, J=7.5 Hz), 1.70 (1H, m), 1.84 (1H, m), 3.80 (3H, s), 4.17 (1H, dd, J=7.5 Hz);

MASS (ES+): m/e 119.

Preparation 116

To a stirred solution of methyl (2R)-2-hydroxybutanoate (1.74 g) in N,N-dimethylformamide (15 ml) was added a solution of tert-butyldiphenylchlorosilane (4.05 g) in N,N-dimethylformamide (5 ml) followed by imidazole (1.05 g) at ambient temperature. The resulting mixture was stirred at the same temperature for three hours and the reaction mixture was poured into ice water and extracted with ethyl acetate. The organic layer was washed with water and brine, and dried over magnesium sulfate. The organic layer was filtered and concentrated in vacuo to give crude methyl-(2R)-2-tert-butyldiphenylsilyloxybutanoate (5.11 g), which was used directly for the next step without further purification.

¹H-NMR (300 MHz, CDCl₃, δ): 0.91 (3H, t, J=7.5 Hz), 1.10 (3×3H, s), 1.74 (2H, dq, J=7.5, 5 Hz), 3.48 (3H, s), 4.20 (1H, t, J=5 Hz), 7.32-7.46 (6H, m), 7.59-7.75 (4H, m);

MASS (ES+) m/e 357.

Preparation 117

To a stirred solution of dimethyl methylphosphonate (8.87 g) in tetrahydrofuran (50 ml) was added n-butyllithium (1.6 M in hexane, 45 ml) dropwise at −78° C. over twenty minutes and the resulting mixture was stirred at the same temperature for half an hour. To this was added a solution of methyl (2R)-2-tert-butyldiphenylsilyloxybutanoate in tetrahydrofuran (30 ml) dropwise at the same temperature over twenty minutes. The mixture was stirred at the same temperature for two hours and allowed to warm to 0° C. The reaction mixture was poured into saturated ammonium chloride and extracted twice with ethyl acetate. The combined organic extracts were washed twice with water and brine, and dried over magnesium sulfate. The organic layer was filtered and concentrated in vacuo. The crude product was purified by flash chromatography eluting with 50% ethyl acetate/hexane (v/v) as a solvent mixture to give dimethyl (3R)-3-tert-butyldiphenylsilyloxy-2-oxopentylphosphate (3.06 g) as a pale yellow oil.

¹H-NMR (300 MHz, CDCl₃, δ): 0.80 (3H, t, J=7.5 Hz), 1.11 (3×3H, s), 1.63 (2H, m), 2.91 (1H, dd, J=22, 16 Hz), 3.35 (1H, dd, J=20, 16 Hz), 3.70 (3H, d, J=2 Hz), 3.74 (3H, d, J=2 Hz), 4.15 (1H, m), 7.32-7.48 (6H, m), 7.56-7.67 (4H, m);

MASS (ES+) m/e 447.

Preparation 118

Compound (118) was obtained in a manner similar to Preparation 1.

¹H-NMR (300 MHz, CDCl₃, δ): 1.41 (3×3H, s), 2.96 (2H, m), 4.50 (1H, m), 5.16 (1H, d, J=8.5 Hz), 6.54 (1H, d, J=7.5 Hz), 6.62-6.82 (2H, m);

MASS (ES−): m/e 296.

Preparation 119

To a stirred solution of (2S)-tert-butoxycarbonylamino-3-(3,4-dihydroxyphenyl)propanoic acid (13.66 g) in N,N-dimethylformamide (150 ml) was added potassium carbonate (22.9 g) at 0° C. and the resulting mixture was stirred at the same temperature for half an hour. To this mixture was added methyl iodide (21.5 g) at the same temperature and the reaction mixture was left at ambient temperature for 2 days. The mixture was poured into water and extracted with ethyl acetate. The organic layer was washed with water and brine, and dried over magnesium sulfate. The organic layer was filtered and concentrated in vacuo. The residue was purified by flash chlomatography eluting with 25 to 50% ethyl acetate/hexane (v/v) as a solvent mixture to give pure methyl (2S)-2-tert-butoxycarbonylamino-3-(3,4-dimethoxyphenyl)-propanoate (7.17 g) as a brown oil.

¹H-NMR (300 MHz, CDCl₃, δ): 1.42 (3×3H, s), 3.01 (1H, dd, J=14, 5.5 Hz), 3.06 (1H, dd, J=14, 5.5 Hz), 3.72 (3H, s), 3.86 (2×3H, s), 4.56 (1H, ddd, J=8.5, 5.5, 5.5 Hz), 4.97 (1H, brd, J=8.5 Hz), 6.64 (1H, s), 6.66 (1H, d, J=8 Hz), 6.79 (1H, d, J=8 Hz);

MASS (ES+) m/e 340.

Preparation 120

To a stirred solution of methyl (2S)-2-tert-butoxycarbonylamino-3-(3,4-dimethoxyphenyl)propanoate (7.13 g) in methanol (40 ml) was added 1 N sodium hydroxide (40 ml) at ambient temperature and the resulting mixture was stirred at the same temperature for three hours and a half. The solvent was evaporated under reduced pressure and the residue was dissolved in water and extracted with ether. The aqueous layer was separated, acidified to pH 2 with concentrated hydrochloric acid and extracted with ethyl acetate. The organic extract was washed with brine, dried over magnesium sulfate, filtered and concentrated in vacuo. The residue was triturated with 50% ether/hexane (v/v) to give (2S)-2-tert-butoxycarbonylamino-3-(3,4-dimethoxyphenyl)propanoic acid (5.35 g) as a white amorphous solid.

¹H-NMR (300 MHz, CDCl₃, δ): 1.42 (3×3H, s), 3.04 (1H, dd, J=14 and 6 Hz), 3.13 (1H, dd, J=14 and 5.5 Hz), 3.855 (3H, s), 3.862 (3H, s), 4.56 (1H, m), 4.92 (1H, brd, J=7.5 Hz), 6.71 (1H, s), 6.72 (1H, d, J=8 Hz), 6.80 (1H, d, J=8 Hz);

MASS (ES+) m/e 324.

Preparation 121

To a stirred solution of tert-butyl (2R)-1-[(2S)-2-benzyloxycarbonylamino]-3-phenylpropanoylpyrrolidine-2-carboxylate (4.33 g) in methanol (40 ml) was added palladium on carbon (10%, 400 mg) and the mixture was stirred under 3 atm hydrogen atmosphere for eighteen hours. The reaction mixture was filtered through a Celite® pad. The filtrate was evaporated to give crude (1S)-1-benzyl-2-[(2R)-2-tert-butoxycarbonylpyrrolidin-1-yl]-2-oxoethylcarbamic acid (3.26 g) as an amorphous solid, which was used directly for the next step without further purification.

¹H-NMR (300 MHz, CDCl₃, δ): 1.30-2.20 (4H, m), 1.42 (9×⅘H, s), 1.48 (9×⅕H, s), 3.14 (1H, m), 3.37-3.77 (3H, m), 4.17 (1×⅘H, t, J=5 Hz), 4.41 (1×⅕H, br), 4.64 (1×⅘H, m), 4.89 (1×⅕H, m), 7.12-7.45 (5H, m), 8.39 (2×⅕H, br), 8.63 (2×⅘H, br);

MASS (ES+) m/e 319.

Preparation 122

Compound (122) was obtained in a manner similar to Preparation 1.

¹H-NMR (300 MHz, CDCl₃, δ): 0.82-1.88 (13H, m), 1.45 (3×3H, s), 4.34 (1H, dt, J=8.5, 5 Hz), 4.86 (1H, d, J=8, 5 Hz);

MASS (ES−) m/e 270.

Preparation 123

Compound (123) was obtained in a manner similar to Preparation 1.

¹H-NMR (300 MHz, CDCl₃, δ): 1.38 (3×3H, s), 5.10 (1H, d, J=8 Hz), 7.25-7.43 (5H, m), 7.59 (1H, d, J=8 Hz);

MASS (ES−) m/e 250.

Preparation 124

Compound (124) was obtained in a manner similar to Preparation 1.

¹H-NMR (300 MHz, CDCl₃, δ): 0.96 (3H, d, J=7.0 Hz), 0.99 (3H, d, J=7.0 Hz), 1.41-1.49 (1H, m), 1.45 (9H, s), 1.47 (3H, s), 2.28 (1H, brs), 5.04 (1H, brs);

MASS (ES+) m/e 232.10 (M+1).

Preparation 125

Compound (125) was obtained in a manner similar to Preparation 1.

¹H-NMR (300 MHz, CDCl₃, δ): 1.42 (9H, s), 3.21 (1H, s), 3.27 (1H, s), 3.66 (1H, s), 3.72 (1H, s), 5.13 (1H, brs), 7.16-7.28 (4H, m);

MASS (ES−) m/e 276.12 (M−1).

Preparation 126

Compound (126) was obtained in a manner similar to Preparation 1.

¹H-NMR (300 MHz, CDCl₃, δ): 4.84-5.12 (1H, br), 2.19-2.34 (2H, m), 1.70-2.04 (6H, m), 1.44 (9H, s), 1.28-1.49 (1H, m);

MASS (ES+) m/e 230.14 (M+1).

Preparation 127

Compound (127) was obtained in a manner similar to Preparation 15.

¹H-NMR (300 MHz, CDCl₃, δ): 0.87 (3H, d, J=7.0 Hz), 0.91 (3H, d, J=7.0 Hz), 1.36-1.54 (1H, m), 1.41 (9H, s), 1.43 (3H, s), 1.72-1.96 (3H, m), 2.10-2.35 (1H, m), 2.58-2.68 (1H, m), 2.93 (1H, dd, J=12.8, 9.5 Hz), 3.11 (1H, dd, J=12.8, 5.1 Hz), 3.47-3.59 (1H, m), 4.35 (1H, dd, J=8.1, 4.0 Hz), 4.65-4.99 (2H, m), 5.06-5.22 (2H, m), 7.04-7.39 (11H, m);

MASS (ES+) m/e 566.30 (M+1).

Preparation 128

Compound (128) was obtained in a manner similar to Preparation 16.

¹H-NMR (300 MHz, CDCl₃, δ): 0.81 (3H, d, J=6.9 Hz), 0.92 (3H, d, J=6.6 Hz), 1.08-1.99 (11H, m), 1.43 (9H, s), 1.46 (3H, s), 2.22-2.39 (1H, m), 2.72-2.90 (1H, m), 2.95-3.09 (1H, m), 3.52-3.61 (1H, m), 3.93-4.09 (1H, m), 4.30-4.39 (1H, m), 4.31 (2H, t, J=6.6 Hz), 4.69-4.76 (1H, m), 4.95 (1H, dt, J=8.4, 5.9 Hz), 5.10-5.23 (2H, m), 6.78 (1H, s), 7.05-7.37 (11H, m), 7.39-7.48 (2H, m), 7.51-7.61 (1H, m), 8.00-8.07 (2H, m);

MASS (ES+) m/e 799.41 (M+1).

Preparation 129

Compound (129) was obtained in a manner similar to Preparation 18.

¹H-NMR (300 MHz, CDCl₃, δ): 0.55-0.70 (3H, m), 0.80-1.04 (3H, m), 1.29 (3H, s), 1.54-2.22 (12H, m), 2.46-2.62 (1H, m), 2.85-3.09 (2H, m), 3.73-3.88 (1H, m), 4.00-4.39 (3H, m), 4.91-5.04 (1H, m), 7.14-7.31 (6H, m), 7.35-7.45 (2H, m), 7.47-7.57 (1H, m), 7.85 (1H, br), 7.95-8.05 (2H, m), 8.24 (2H, br);

MASS (ES+) m/e 609.3 (Free, M+1).

Preparation 130

Compound (130) was obtained in a manner similar to Preparation 76.

¹H-NMR (300 MHz, CDCl₃, δ): 0.70 (3H, d, J=7.0 Hz), 0.85 (3H, d, J=6.6 Hz), 1.14 (3H, s), 1.32-2.00 (9H, m), 2.10-2.40 (2H, m), 2.99 (1H, dd, J=13.9, 7.0 Hz), 3.20 (1H, dd, J=13.9, 8.8 Hz), 3.26-3.37 (1H, m), 3.82-3.92 (1H, m), 4.18-4.29 (1H, m), 4.31 (2H, t, J=6.6 Hz), 4.65-4.71 (1H, m), 5.15-5.26 (1H, m), 5.75 (1H, s), 7.12 (1H, d, J=10.6 Hz), 7.15-7.31 (5H, m), 7.39-7.47 (2H, m), 7.52-7.62 (2H, m), 7.99-8.06 (2H, m);

MASS (ES+) m/e 591.37 (M+1).

Preparation 131

Compound (131) was obtained in a manner similar to Preparation 77.

¹H-NMR (300 MHz, CDCl₃, δ): 0.70 (3H, d, J=7.0 Hz), 0.88 (3H, d, J=6.6 Hz), 1.15 (3H, s), 1.22-1.94 (9H, m), 2.09-2.37 (2H, m), 2.99 (1H, dd, J=13.9, 7.0 Hz), 3.20 (1H, dd, J=13.9, 8.8 Hz), 3.26-3.37 (2H, m), 3.65 (2H, t, J=6.2 Hz), 3.82-3.93 (1H, m), 4.17-4.28 (1H, m), 4.65-4.72 (1H, m), 5.15-5.26 (1H, m), 5.85 (1H, s), 7.12 (1H, d, J=10.3 Hz), 7.16-7.31 (5H, m), 7.58 (1H, d, J=10.3 Hz);

MASS (ES+) m/e 487.39 (M+1).

Preparation 132

Compound (132) was obtained in a manner similar to Preparation 78. The obtained compound was used in Example 62.

¹H-NMR (300 MHz, CDCl₃, δ): 0.71 (3H, d, J=7.0 Hz), 0.89 (3H, d, J=6.6 Hz), 1.15 (3H, s), 1.48-1.94 (6H, m), 2.09-2.41 (2H, m), 2.43-2.55 (2H, m), 2.99 (1H, dd, J=13.6, 7.0 Hz), 3.20 (1H, dd, J=13.6, 8.8 Hz), 3.25-3.37 (2H, m), 3.89 (1H, dt, J=8.4, 4.8 Hz), 4.24 (1H, ddd, J=10.3, 7.3, 7.0 Hz), 4.66-4.72 (1H, m), 5.21 (1H, m), 7.53 (1H, d, J=10.3 Hz), 9.77 (1H, dd, J=1.1, 1.5 Hz);

MASS (ES+) m/e 485.40 (M+1).

Preparation 133

Compound (133) was obtained in a manner similar to Preparation 15.

¹H-NMR (300 MHz, CDCl₃, δ): 1.35 (9H, s), 1.71-1.95 (3H, m), 2.52-2.61 (1H, m), 2.69 (1H, dd, J=12.8 and 9.5 Hz), 2.90 (1H, dd, J=12.8, 5.1 Hz), 3.02-3.20 (2H, m), 3.23-3.33 (1H, m), 3.43-3.61 (1H, m), 4.31 (1H, dd, J=8.4, 4.3 Hz), 4.41-4.53 (1H, m), 4.90 (1H, dt, J=9.5, 5.1 Hz), 4.95-5.05 (1H, m), 5.08 (1H, d, J=12.5 Hz), 5.17 (1H, d, J=12.5 Hz), 6.68 (1H, d, J=7.3 Hz), 6.96-7.48 (13H, m), 7.63 (1H, s), 7, 74-7.82 (3H, m);

MASS (ES+) m/e 650.50 (M+1).

Preparation 134

Compound (134) was obtained in a manner similar to Preparation 16.

¹H-NMR (300 MHz, CDCl₃, δ): 1.13-1.29 (2H, m), 1.30-1.98 (8H, m), 1.39 (9H, s), 2.58-2.70 (1H, m), 2.71-3.00 (2H, m), 3.08-3.20 (1H, m), 3.21-3.35 (1H, m), 3.47-3.59 (1H, m), 3.97-4.17 (3H, m), 4.27-4.35 (1H, m), 4.79-4.95 (2H, m), 5.03-5.18 (1H, m), 5.09 (1H, d, J=12.5 Hz), 5.16 (1H, d, J=12.5 Hz), 6.74-6.92 (1H, m), 7.07-7.46 (16H, m), 7.50-7.59 (1H, m), 7.61 (1H, s), 7.72-7.79 (3H, m), 8.01 (2H, d, J=7.7 Hz):

MASS (ES+) m/e 883.63 (M+1).

Preparation 135

Compound (135) was obtained in a manner similar to Preparation 17.

¹H-NMR (300 MHz, CDCl₃, δ): 1.09-2.11 (10H, m), 1.38 (−9H, s), 2.60-2.73 (1H, m), 2.72-2.82 (1H, m), 2.83-2.96 (1H, m), 3.10-3.21 (1H, m), 3.24-3.39 (1H, m), 3.59-3.76 (1H, m), 3.99-4.14 (3H, m), 4.20-4.36 (1H, m), 4.71-4.95 (2H, m), 5.26-5.36 (1H, m), 7.05-7.15 (1H, m), 7.16-7.26 (5H, m), 7.27-7.34 (1H, m), 7.35-7.47 (4H, m), 7.50-7.64 (3H, m), 7.70-7.80 (3H, m), 7.97-8.06 (2H, m);

MASS (ES+) m/e 793.47 (M+1).

Preparation 136

Compound (136) was obtained in a manner similar to Preparation 18.

¹H-NMR (300 MHz, CDCl₃, δ): 0.83-1.91 (10H, m), 2.45-3.11 (4H, m), 3.14-3.32 (1H, m), 3.55-3.69 (1H, m), 3.75-3.94 (2H, m), 4.04-4.14 (1H, m), 4.18-4.34 (1H, m), 4.47-4.64 (1H, m), 5.11-5.25 (1H, m), 7.03-7.55 (14H, m), 7.62-7.81 (3H, m), 7.85-8.15 (4H, m), 8.38 (1H, br);

MASS (ES+) m/e 693.47 (free, M+1).

Preparation 137

Compound (137) was obtained in a manner similar to Preparation 76.

¹H-NMR (300 MHz, CDCl₃, δ): 1.18-1.53 (2H, m), 1.59-1.94 (3H, m), 2.06-2.40 (5H, m), 2.86 (1H, dd, J=13.2, 5.1 Hz), 3.01 (1H, dd, J=13.9, 7.0 Hz), 3.03-3.15 (1H, m), 3.18 (1H, dd, J=13.2, 10.6 Hz), 3.39 (1H, dd, J=13.9, 8.4 Hz), 3.90-4.00 (1H, m), 4.19-4.35 (1H, m), 4.25 (2H, t, J=6.6 Hz), 4.59-4.65 (1H, m), 4.81-4.91 (1H, m), 5.07 (1H, dt, J=10.6, 5.1 Hz), 6.33 (1H, d, J=9.9 Hz), 6.47 (1H, d, J=10.6 Hz), 7.13-7.29 (5H, m), 7.34 (1H, dd, J=8.4, 1.5 Hz), 7.37-7.49 (5H, m), 7.52-7.59 (1H, m), 7.67 (1H, s), 7.73-7.83 (3H, m), 7.99-8.05 (2H, m);

MASS (ES+) m/e 675.50 (M+1).

Preparation 138

Compound (138) was obtained in a manner similar to Preparation 77.

¹H-NMR (300 MHz, CDCl₃, δ): 1.20-1.89 (9H, m), 2.14-2.39 (2H, m), 2.85 (1H, dd, J=13.6 and 5.1 Hz), 3.00 (1H, dd, J=14.3 and 6.6 Hz), 3.04-3.13 (1H, m), 3.17 (1H, dd, J=13.6 and 10.6 Hz), 3.38 (1H, dd, J=14.3, 8.4 Hz), 3.57 (2H, t, J=6.2 Hz), 3.90-3.99 (1H, m), 4.28 (1H, dt, J=10.3, 7.7 Hz), 4.58-4.65 (1H, m), 4.80-4.90 (1H, m), 5.06 (1H, dt, J=10.6, 5.1 Hz), 6.39 (1H, d, J=9.9 Hz), 6.48 (1H, d, J=10.6 Hz), 7.12-7.28 (6H, m), 7.34 (1H, dd, J=10.3, 1.8 Hz), 7.41-7.50 (2H, m), 7.67 (1H, m), 7.73-7.83 (3H, m);

MASS (ES+) m/e 571.35 (M+1).

Preparation 139

Compound (139) was obtained in a manner similar to Preparation 78.

¹H-NMR (300 MHz, CDCl₃, δ): 1.45-1.88 (6H, m), 2.13-2, 48 (4H, m), 2.86 (1H, dd, J=13.6, 5.1 Hz), 3.02 (1H, dd, J=14.3, 7.0 Hz), 3.06-3.16 (1H, m), 3.18 (1H, dd, J=13.6, 10.6 Hz), 3.40 (1H, dd, J=14.3, 8.4 Hz), 3.91-4.01 (1H, m), 4.29 (1H, dt, J=10.3, 7.0 Hz), 4.58-4.67 (1H, m), 4.80-4.92 (1H, m), 5.07 (1H, dt, J=10.6, 5.1 Hz), 6.33 (1H, d, J=9.9 Hz), 6.44 (1H, d, J=10.3 Hz), 7.13-7.29 (6H, m), 7.35 (1H, dd, J=8.4, 1.5 Hz), 7.40-7.52 (2H m), 7.67 (1H, s), 7.74-7.85 (3H, m), 9.69 (1H, s);

MASS (ES+) m/e 569.35 (M+1).

Preparation 140

Compound (140) was obtained in a manner similar to Preparation 15.

¹H-NMR (300 MHz, CDCl₃, δ): 1.36-1.54 (1H, m), 1.43 (9H, s), 1.71-1.98 (3H, m), 2.55-2.66 (1H, m), 2.86-3.11 (3H, m), 3.44-3.62 (1H, m), 3.45 (2H, d, J=16.6 Hz), 3.76 (2H, d, J=16.6 Hz), 4.34-4.40 (1H, m), 4.98 (1H, ddd, J=9.5, 8.8, 5.1 Hz), 5.04-5.14 (1H, m), 5.10 (1H, d, J=12.5 Hz), 5.19 (1H, d, J=12.5 Hz), 7.07 (1H, d, J=8.8 Hz), 7.12-7.30 (8H, m), 7.30-7.40 (5H, m);

MASS (ES+) m/e 612.49 (M+1).

Preparation 141

Compound (141) was obtained in a manner similar to Preparation 16.

¹H-NMR (300 MHz, CDCl₃, δ): 1.37 (6H, s), 1.43 (3H, s), 1.48-2.03 (10H, m), 2.66-2.78 (1H, m), 2.84-3.05 (2H, m), 3.13-3.26 (1H, m), 3.27-3.49 (2H, m), 3.53-3.67 (2H, m), 3.92-4.06 (1H, m), 4.17-4.38 (3H, m), 4.88-5.00 (1H, m), 5.07-5.27 (3H, m), 6.86-6.97 (1H, m), 7.09-7.37 (15H, m), 7.38-7.47 (2H, m), 7.51-7.59 (1H, m), 7.98-8.06 (2H, m); MASS (ES+) m/e 845.56 (M+1).

Preparation 142

Compound (142) was obtained in a manner similar to Preparation 17.

¹H-NMR (300 MHz, CDCl₃, δ): 1.38 (6H, s), 1.45 (3H, s), 1.50-1.89 (8H, m), 1.88-2.19 (1H, m), 2.65-2.79 (1H, m), 2.95-3.34 (4H, m), 3.45-3.76 (4H, m), 3.92-4.05 (1H, m), 4.17-4.39 (4H, m), 4.78-4.92 (1H, m), 5.13-5.35 (1H, m), 7.00-7.32 (10H, m), 7.39-7.60 (4H, m), 7.98-8.07 (2H, m);

MASS (ES+) m/e 755.32 (M+1).

Preparation 143

Compound (143) was obtained in a manner similar to Preparation 18.

¹H-NMR (300 MHz, CDCl₃, δ): 1.17-1.46 (2H, m), 1.53-2.16 (8H, m), 2.86-3.14 (2H, m), 3.26-3.78 (6H, m), 4.03-4.32 (4H, m), 4.89-5.01 (1H, m), 7.00-7.31 (9H, m), 7.33-7.43 (2H, m), 7.47-7.55 (1H, m), 7.73 (1H, brs), 7.94-8.14 (4H, m), 8.30 (1H, brs), 8.86 (1H, brs);

MASS (ES+) m/e 655.37 (free, M+1).

Preparation 144

Compound (144) was obtained in a manner similar to Preparation 76.

¹H-NMR (300 MHz, CDCl₃, δ): 1.30-1.58 (4H, m), 1.66-1.94 (6H, m), 2.10-2.39 (2H, m), 2.93 (1H, dd, J=13.2, 5.1 Hz), 3.09-3.21 (1H, m), 3.30 (1H, dd, J=13.2, 10.3 Hz), 3.61 (1H, d, J=16.5 Hz), 3.89-4.01 (1H, m), 3.94 (2H, d, J=16.5 Hz), 4.17-4.38 (3H, m), 4.63-4.69 (1H, m), 5.14 (1H, dt, J=10.3, 5.1 Hz), 6.31 (1H, s), 7.05-7.31 (9H, m), 7.37-7.57 (4H, m), 7, 99-8.04 (2H, m);

MASS (ES+) m/e 637.30 (M+1).

Preparation 145

Compound (145) was obtained in a manner similar to Preparation 77.

¹H-NMR (300 MHz, CDCl₃, δ): 1.21-1.89 (9H, m), 2.08-2.39 (2H, m), 2.85 (1H, d, J=16.8 Hz), 2.93 (1H, dd, J=13.2, 5.1 Hz), 3.10-3.21 (1H, m), 3.30 (1H, dd, J=13.2, 10.3 Hz). 3.62 (1H, d, J=16.8 Hz), 3.63 (2H, t, J=6.2 Hz), 3.89-4.00 (1H, m), 3.97 (2H, d, J=16.8 Hz), 4.22 (1H, dt, J=10.3, 7.7 Hz), 4.64-4.70 (1H, m), 5.14 (1H, dt, J=10.3, 5.1 Hz), 6.51 (1H, s), 7.12-7.30 (10H, m), 7.52 (1H, d, J=10.3 Hz);

MASS (ES+) m/e 533.34 (M+1).

Preparation 146

Compound (146) was obtained in a manner similar to Preparation 78. The obtained compound was used in Example 68.

¹H-NMR (300 MHz, CDCl₃, δ): 1.46-1.87 (6H, m), 2.07-2.44 (2H, m), 2.46 (2H, dt, J=7.0, 1.5 Hz), 2.86 (1H, d, J=16.2 Hz), 2.92 (1H, dd, J=13.2, 5.1 Hz), 3.08-3.20 (1H, m), 3.29 (1H, dd, J=13.2, 10.6 Hz), 3.61 (1H, d, J=16.2 Hz), 3.87-4.00 (1H, m), 3.96 (2H, d, J=16.2 Hz), 4.23 (1H, ddd, J=10.3, 7.7, 7.0 Hz), 4.62-4.71 (1H, m), 5.14 (1H, dt, J=10.6, 5.1 Hz), 6.44 (1H, s), 7.13-7.31 (10H, m), 7.48 (1H, d, J=10.3 Hz), 9.73 (1H, t, J=1.5 Hz);

MASS (ES+) m/e 531.28 (M+1).

Preparation 147

Compound (147) was obtained in a manner similar to Preparation 14.

¹H-NMR (300 MHz, CDCl₃, δ): 1.18-1.51 (2H, m), 1.42 (9H, s), 1.57-1.83 (2H, m), 2.48-2.58 (1H, m), 3.11 (1H, dd, J=12.8, 9.5 Hz), 3.23 (1H, dd, J=12.8, 5.3 Hz), 3.41-3.52 (1H, m), 4.31-4.39 (1H, m), 4.72 (1H, dt, J=9.5, 5.3 Hz), 5.09 (1H, d, J=12.5 Hz), 5.19 (1H, d, J=12.5 Hz), 5.43 (1H, d, J=8.8 Hz), 7.26-7.39 (5H, m), 7.39-7.49 (2H, m), 7.66 (1H, s), 7.69-7.81 (4H, m);

MASS (ES+) m/e 503.38 (M+1).

Preparation 148

Compound (148) was obtained in a manner similar to Preparation 15.

¹H-NMR (300 MHz, CDCl₃, δ): 0.80 (3H, t, J=7.3 Hz), 1.20-2.06 (3H, m), 1.36 (3H, s), 1.41 (2H, s), 1.44 (7H, s), 2.55-2.66 (1H, m), 3.12 (1H, dd, J=12.8, 9.2 Hz), 3.18-3.28 (1H, m), 3.23 (1H, dd, J=12.8, 5.1 Hz), 3.45-3.62 (2H, m), 4.33-4.39 (1H, m), 4.97-5.16 (2H, m), 5.09° (1H, d, J=12.5 Hz), 5.15 (1H, d, J=12.5 Hz), 6.90 (1H, d, J=8.4 Hz), 7.28-7.49 (8H, m), 7.67 (1H, s), 7.70-7.81 (4H, m);

MASS (ES+) m/e 602.46 (M+1).

Preparation 149

Compound (149) was obtained in a manner similar to Preparation 16.

¹H-NMR (300 MHz, CDCl₃, δ): 0.72 (3H, t, J=7.3 Hz), 1.31-2.07 (11H, m), 1.43 (9H, s), 1.48 (3H, s), 2.13-2.32 (1H, m), 2.68-2.78 (1H, m)_{r 3.17} (2H, d, J=7.3 Hz), 3.52-3.63 (1H, m), 4.00-4.12 (1H, m), 4.31 (2H, t, J=6.2 Hz), 4.35-4.40 (1H, m), 4.92-5.23 (4H, m), 6.73-6.92 (1H, m), 6.97 (1H, s), 7.24-7.49 (12H, m), 7.51-7.82 (3H, m), 8.00-8.06 (2H, m);

MASS (ES+) m/e 835.60 (M+1).

Preparation 150

Compound (150) was obtained in a manner similar to Preparation 17.

¹H-NMR (300 MHz, CDCl₃, δ): 0.76 (3H, t, J=7.0 Hz), 1.43 (9H, s), 1.58-1.98 (15H, m), 2.65-2.78 (1H, m), 3.04-3.28 (2H, m), 3.65-3.77 (1H, m), 4.05-4.15 (1H, m), 4.22-4.38 (3H, m), 4.93-5.05 (1H, m), 5.50-5.60 (1H, m), 6.81 (1H, s), 7.22-7.58 (7H, m), 7.65 (1H, s), 7.68-7.83 (3H, m), 7.98-8.05 (2H, m);

MASS (ES+) m/e 745.52 (M+1).

Preparation 151

Compound (151) was obtained in a manner similar to Preparation 18.

¹H-NMR (300 MHz, CDCl₃, δ): 0.59-0.74 (3H, m), 1.07-2.19 (13H, m), 1.37 (3H, s), 2.91-3.31 (3H, m), 3.65-3.78 (1H, m), 4.06-4.38 (4H, m), 4.99-5.10 (1H, m), 7.21-7.54 (7H, m), 7.60-7.78 (4H, m), 7.94-8.02 (2H, m), 8.08-8.49 (3H, m);

MASS (ES+) m/e 645.57 (free, M+1).

Preparation 152

Compound (152) was obtained in a manner similar to Preparation 76.

¹H-NMR (300 MHz, CDCl₃, δ): 0.81 (3H, t, J=7.0 Hz), 1.28 (3H, s), 1.36-1.56 (2H, m), 1.62-1.99 (6H, m), 2.07-2.22 (2H, m), 2.22-2.41 (2H, m), 3.12 (1H, dd, J=13.6, 5.9 Hz), 3.18-3.30 (1H, m), 3.41 (1H, dd, J=13.6, 9.9 Hz), 3.81-3.92 (1H, m), 4, 19-4.31 (1H, m), 4.32 (2H, t, J=6.2 Hz), 4.61-4.68 (1H, m), 5.30 (1H, dt, J=9.9, 5.9 Hz), 5.91 (1H, s), 7.16 (1H, d, J=10.6 Hz), 7.35-7.49 (5H, m), 7.51-7.59 (1H, m), 7.64 (1H, d, J=9.9 Hz), 7.69 (1H, s), 7.73-7.83 (3H, m), 8.00-8.06 (2H, m);

MASS (ES+) m/e 627.44 (M+1).

Preparation 153

Compound (153) was obtained in a manner similar to Preparation 77.

¹H-NMR (300 MHz, CDCl₃, δ): 0.84 (3H, t, J=7.3 Hz), 1.28-1.52 (2H, m), 1.29 (3H, s), 1.53-1.96 (7H, m), 2.08-2.25 (2H, m), 2.25-2.41 (2H, m), 3.13 (1H, dd, J=13.6, 5.9 Hz), 3.19-3.30 (1H, m), 3, 42 (1H, dd, J=13.6, 9.9 Hz), 3.67 (2H, t, J=6.6 Hz), 3.82-3.92 (1H, m), 4.24 (1H, dt, J=10.3, 7.3 Hz), 4.61-4.68 (1H, m), 5.30 (1H, dt, J=9.9, 5.9 Hz), 5.95 (1H, s), 7.15 (1H, d, J=10.3 Hz), 7.35-7.50 (3H, m), 7.63 (1H, d, J=10.3 Hz), 7.69 (1H, s), 7.72-7.83 (3H, m);

MASS (ES+) m/e 523.38 (M+1).

Preparation 154

Compound (154) was obtained in a manner similar to Preparation 78. The obtained compound was used in Example 71.

¹H-NMR (300 MHz, CDCl₃, δ): 0.83 (3H, t, J=7.3 Hz), 1.29 (3H, s), 1.49-1.93 (6H, m), 2.08-2.23 (2H, m), 2.24-2.39 (2H, m), 2.45-2.55 (2H, m), 3.12 (1H, dd, J=13.6, 5.9 Hz), 3.18-3.29 (1H, m), 3.41 (1H, dd, J=13.6, 9.9 Hz), 3.82-3.93 (1H, m), 4.18-4.30 (1H, m), 4.61-4.68 (1H, m), 5.30 (1H, dt, J=9.9, 5.9 Hz), 5.87 (1H, s), 7.15 (1H, d, J=10.3 Hz), 7.37 (1H, dd, J=8.4, 1.8 Hz), 7.42-7.49 (2H, m), 7.57 (1H, d, J=10.3 Hz), 7.69 (1H, s), 7.74-7.83 (3H, m), 9.77 (1H, s);

MASS (ES+) m/e 521.33 (M+1).

Preparation 155

Compound (155) was obtained in a manner similar to Preparation 15.

¹H-NMR (300 MHz, CDCl₃, δ): 1.40 (9H, s), 1.40-1.50 (1H, m), 1.71-1.96 (3H, m), 2.50-2.85 (3H, m), 2.95-3.28 (2H, m), 3.45-3.60 (1H, m), 3.72 (3H, s), 4.30 (1H, dd, J=7.3, 4.1 Hz), 4.39-4.51 (1H, m), 4.81-4.92 (1H, m), 5.00-5.20 (1H, m), 5.07 (1H, d, J=12.1 Hz), 5.14 (1H, d, J=12.1 Hz), 6.58 (1H, d, J=8.1 Hz), 6.88 (1H, s), 7.08-7.37 (13H, m), 7.63 (1H, d, J=8.1 Hz);

MASS (ES+) m/e 653.51 (M+1).

Preparation 156

Compound (156) was obtained in a manner similar to Preparation 16.

¹H-NMR (300 MHz, CDCl₃, δ): 1.26-1.97 (14H, m), 1.39 (9H, s), 2.53-2.78 (2H, m), 2.94-3.31 (2H, m), 3.49-3.62 (1H, m), 3.71 (3H, s), 3.94-4.05 (1H, m), 4.16-4.36 (2H, m), 4.67-4.84 (1H, m), 4.99-5.19 (1H, m), 5.06 (1H, d, J=12.5 Hz), 5.13 (1H, d, J=12.5 Hz), 6.62-6.77 (1H, m), 6.86 (1H, s), 7.01-7.46 (15H, m), 7.50-7.57 (1H, m), 7.64 (1H, d, J=7.7 Hz), 7.99-8.06 (2H, m);

MASS (ES+) m/e 886.62 (M+1).

Preparation 157

Compound (157) was obtained in a manner similar to Preparation 17.

¹H-NMR (300 MHz, CDCl₃, δ): 1.16-2.14 (12H, m), 1.38 (9H, s), 2.49-2.67 (2H, m), 2.73-2.85 (1H, m), 3.08-3.28 (2H, m), 3.53-3.72 (1H, m), 3.72 (3H, s), 3.86-3.97 (1H, m), 4.18-4.35 (3H, m), 4.43-4.59 (1H, m), 4.60-4.74 (1H, m), 5.46 (1H, brs), 6.91 (1H, s), 7.00-7.12 (3H, m), 7.15-7.32 (5H, m), 7.38-7.47 (2H, m), 7.51-7.61 (2H, m), 7.99-8.06 (2H, m);

MASS (ES+) m/e 796.59 (M+1).

Preparation 158

Compound (158) was obtained in a manner similar to Preparation 18.

¹H-NMR (300 MHz, CDCl₃, δ): 1.03-1.20 (2H, m), 1.36-1.96 (11H, m), 2.63-3.23 (6H, m), 3.65 (3H, s), 3.97-4.20 (3H, m), 4.41-4.55 (1H, m), 4.96-5.13 (1H, m), 7.01-7.31 (9H, m), 7.36-7.45 (2H, m), 7.46-7.57 (1H, m), 7.62-7.70 (1H, m), 7.88-8.24 (4H, m);

MASS (ES+) m/e 696.53 (free, M+1).

Preparation 159

Compound (159) was obtained in a manner similar to Preparation 76.

¹H-NMR (300 MHz, CDCl₃, δ): 1.34-1.53 (2H, m), 1.63-1.95 (6H, m), 2.13-2.38 (2H, m), 2.86 (1H, dd, J=13.2, 5.5 Hz), 3.00 (1H, dd, J=14.2, 6.6 Hz), 3.04-3.22 (1H, m), 3.17 (1H, dd, J=13.2, 9.9 Hz), 3.35 (1H, dd, J=14.2, 11.0 Hz), 3.70 (3H, s), 3.87-4.06 (1H, m), 4.28 (2H, t, J=6.6 Hz), 4.29-4.37 (1H, m), 4.59-4.65 (1H, m), 4.78-4.88 (1H, m), 5.08 (1H, dt, J=11.0, 5.5 Hz), 6.42 (1H, d, J=9.9 Hz), 6.54 (1H, d, J=11.0 Hz), 6.87 (1H, s), 7.08-7.31 (9H, m), 7.40-7.49 (2H, m), 7.55 (1H, d, J=7.7 Hz), 7.59 (1H, d, J=7.7 Hz), 8.00-8.07 (2H, m);

MASS (ES+) m/e 678.40 (M+1).

Preparation 160

Compound (160) was obtained in a manner similar to Preparation 77.

¹H-NMR (300 MHz, CDCl₃, δ): 1.21-1.92 (9H, m), 2.10-2.39 (2H, m), 2.86 (1H, dd, J=14.7, 6.6 Hz), 2.99 (1H, dd, J=13.6, 5.5 Hz), 3.04-3.15 (1H, m), 3.17 (1H, dd, J=13.6, 10.6 Hz), 3.34 (1H, dd, J=14.7, 9.2 Hz), 3.62 (2H, t, J=6.2 Hz), 3.72 (3H, s), 3.91-4.01 (1H, m), 4.29 (1H, dt, J=10.3, 7.7 Hz), 4.59-4.65 (1H, m), 4.81 (1H, dt, J=9.2, 6.6 Hz), 5.08 (1H, dt, J=10.6, 5.5 Hz), 6.44 (1H, d, J=10.3 Hz), 6.48 (1H, d, J=10.6 Hz), 6.87 (1H, s), 7.08-7.31 (9H, m), 7.60 (1H, dd, J=8.1, 0.7 Hz);

MASS (ES+) m/e 574.42 (M+1).

Preparation 161

Compound (161) was obtained in a manner similar to Preparation 78. The obtained compound was used in Example 74.

¹H-NMR (300 MHz, CDCl₃, δ): 1.40-1.91 (5H, m), 2.14-2.40 (2H, m), 2.44 (2H, dt, J=6.6, 1.5 Hz), 2.86 (1H, dd, J=13.2, 10.6 Hz), 2.99 (1H, dd, J=14.7, 6.2 Hz), 3.04-3.15 (1H, m), 3.17 (1H, dd, J=13.2, 10.6 Hz), 3.34 (1H, dd, J=14.7, 8.4 Hz), 3.73 (3H, s), 3.92-4.01 (1H, m), 4.29 (1H, dt, J=10.3, 7.3 Hz), 4.58-4.65 (1H, m), 4.81 (1H, dt, J=9.9, 6.2 Hz), 5.08 (1H, dt, J=10.6, 5.1 Hz), 6.33 (1H, d, J=10.3 Hz), 6.43 (1H, d, J=10.3 Hz), 6.87 (1H, s), 7.07-7.36 (9H, m), 7.60 (1H, s, J=7.7 Hz), 9.73 (1H, s);

MASS (ES+) m/e 572.35 (M+1).

Preparation 162

Compound (162) was obtained in a manner similar to Preparation 14.

¹H-NMR (300 MHz, CDCl₃, δ): 1.43 (9H, s), 1.45-1.61 (1H, m), 1.76-2.00 (3H, m), 2.29 (3H, s), 2.63-2.75 (1H, m), 2.84-3.06 (2H, m), 3.48-3.66 (1H, m), 4.32-4.39 (1H, m), 4.56-4.66 (1H, m), 5.07-5.23 (2H, m), 5.33-5.42 (1H, m), 7.02-7.12 (4H, m), 7.30-7.39 (5H, m);

MASS (ES+) m/e 467.38 (M+1).

Preparation 163

Compound (163) was obtained in a manner similar to Preparation 15.

¹H-NMR (300 MHz, CDCl₃, δ): 0.80 (3H, t, J=7.5 Hz), 1.38 (3H, s), 1.41 (3H, s), 1.43 (6H, s), 1.45-1.64 (2H, m), 1.75-2.14 (4H, m), 2.30 (3H, s), 2.69-2.84 (1H, m), 2.91 (1H, dd, J=13.2, 9.0 Hz), 3.03 (1H, dd, J=13.2, 5.7 Hz), 3.50-3.61 (1H, m), 4.34-4.40 (1H, m), 4.93 (1H, dt, J=9.0, 5.7 Hz), 5.04-5.24 (3H, m), 6.88 (1H, d, J=9.0 Hz), 6.93-7.13 (5H, m), 7.29-7.40 (5H, m);

MASS (ES+) m/e 566.52 (M+1).

Preparation 164

Compound (164) was obtained in a manner similar to Preparation 16.

¹H-NMR (300 MHz, CDCl₃, δ): 0.72 (3H, t, J=6.6 Hz), 1.38-2.00 (13H, m), 1.44 (9H, s), 1.49 (3H, s), 2.30 (3H, s), 2.75-3.00 (2H, m), 3.53-3.64 (1H, m), 3.98-4.12 (1H, m), 4.32 (2H, t, J=6.6 Hz), 4.39 (1H, dd, J=8.2, 4.4 Hz), 4.85-4.96 (1H, m), 5.06-5.19 (3H, m), 6.67-6.82 (1H, m), 6.91-7.01 (1H, m), 7.04-7.11 (4H, m), 7.29-7.37 (5H, m), 7.39-7.47 (2H, m), 7.51-7.60 (1H, m), 8.00-8.06 (2H, m);

MASS (ES+) m/e 799.47 (M+1).

Preparation 165

Compound (165) was obtained in a manner similar to Preparation 17.

¹H-NMR (300 MHz, CDCl₃, δ): 0.79 (3H, t, J=10.5 Hz), 1.45 (12H, s), 1.46-1.96 (12H, m), 2.11-2.24 (1H, m), 2.32 (3H, s), 2.72-2.84 (1H, m), 2.89-3.07 (2H, m), 3.65-3.76 (1H, m), 4.00-4.12 (1H, m), 4.26-4.40 (3H, m), 4.83-4.94 (1H, m), 5.38 (1H, brs), 6.78 (1H, s), 7.07-7.12 (4H, m), 7.16-7.22 (1H, d, J=8.1 Hz), 7.40-7.48 (2H, m), 7.52-7.60 (1H, m), 8.01-8.07 (2H, m);

MASS (ES+) m/e 709.38 (M+1).

Preparation 166

Compound (166) was obtained in a manner similar to Preparation 18.

¹H-NMR (300 MHz, CDCl₃, δ): 0.64-0.75 (3H, m), 1.37 (3H, s), 1.54-2.14 (12H, m), 2.27 (3H, s), 2.81-3.07 (4H, m), 3.67-3.80 (1H, m), 4.17-4.37 (4H, m), 4.85-4.96 (1H, m), 7.00-7.12 (4H, m), 7.36-7.44 (2H, m), 7.49-7.64 (2H, m), 7.97-8.04 (2H, m), 8.07-8.43 (3H, m);

MASS (ES+) m/e 609.40 (free, M+1).

Preparation 167

Compound (167) was obtained in a manner similar to Preparation 76.

¹H-NMR (300 MHz, CDCl₃, δ): 0.82 (3H, t, J=7.3 Hz), 1.28 (3H, s), 1.36-1.57 (2H, m), 1.62-1.98 (5H, m), 2.06-2.40 (4H, m), 2.30 (3H, s), 2.92 (1H, dd, J=13.6, 6.3 Hz), 3.15-3.33 (2H, m), 3.82-3.91 (1H, m), 4.25 (1H, dt, J=10.5, 7.7 Hz), 4.32 (2H, t, J=6.3 Hz), 4.64-4.70 (1H, m), 5.17 (1H, dt, J=10.5, 6.3 Hz), 5.85 (1H, s), 7.04-7.16 (5H, m), 7.15 (1H, d, J=10.5 Hz), 7.40-7.48 (2H, m), 7.50-7.60 (2H, m), 8.01-8.06 (2H, m);

MASS (ES+): m/e 591.56 (M+1).

Preparation 168

Compound (168) was obtained in a manner similar to Preparation 77.

¹H-NMR (300 MHz, CDCl₃, δ): 0.84 (3H, t, J=7.3 Hz), 1.22-1.94 (9H, m), 1.28 (3H, s), 2.07-2.40 (4H, m), 2.30 (3H, s), 2.91 (1H, dd, J=13.2, 6.2 Hz), 3.20 (1H, dd, J=13.2, 9.9 Hz), 3.22-3.32 (1H, m), 3.66 (2H, t, J=6.3 Hz), 3.81-3.91 (1H, m), 4.23 (1H, dt, J=10.3, 7.7 Hz), 4.63-4.70 (1H, m), 5.16 (1H, dt, J=10.3, 6.2 Hz), 5.93 (1H, s), 7.04-7.14 (4H, m), 7.14 (1H, d, J=9.9 Hz), 7.53 (1H, d, J=10.3 Hz);

MASS (ES+): m/e 487.46 (M+1).

Preparation 169

Compound (169) was obtained in a manner similar to Preparation 78. The obtained compound was used in Example 77.

¹H-NMR (300 MHz, CDCl₃, δ): 0.84 (3H, t, J=7.3 Hz), 1.29 (3H, s), 1.49-1.91 (6H, m), 2.08-2.39 (4H, m), 2.30 (3H, s), 2.45-2.54 (2H, t, J=6.3 Hz), 2.91 (1H, dd, J=13.6, 5.9 Hz), 3.20 (1H, dd, J=13.6, 10.3 Hz), 3.22-3.32 (1H, m), 3.81-3.91 (1H, m), 4.23 (1H, dt, J=10.6, 7.0 Hz), 4.64-4.70 (1H, m), 5.15 (1H, dt, J=10.3, 5.9 Hz), 5.87 (1H, s), 7.05-7.14 (4H, m), 7.15 (1H, d, J=10.6 Hz), 7.48 (1H, d, J=10.3 Hz), 9.77 (1H, s);

MASS (ES+): m/e 485.39 (M+1).

Preparation 170

Compound (170) was obtained in a manner similar to Preparation 14.

¹H-NMR (300 MHz, CDCl₃, δ): 1.32-1.38 (9H, m), 1.56-2.31 (3H, m), 3.01-3.23 (2H, m), 3.33-3.43 (1H, m), 3.57-3.80 (1H, m), 4.36-4.44 (1H, m), 4.84-4.96 (1H, m), 5.05-5.23 (3H, m), 5.35-5.43 (1H, m), 7.07-7.20 (2H, m), 7.27-7.40 (5H, m), 7.49-7.62 (1H, m), 8.46-8.56 (1H, m);

MASS (ES+): m/e 454.31 (M+1).

Preparation 171

Compound (171) was obtained in a manner similar to Preparation 15.

¹H-NMR (300 MHz, CDCl₃, δ): 0.763 (3H, t, J=6.3 Hz), 1.33-1.53 (2H, m), 1.36-1.40 (3H, m), 1.42 (9H, s), 1.73-2.37 (4H, m), 3.03-3.30 (2H, m), 3.35-3.87 (2H, m), 4.40-4.45 (1H, m), 5.06-5.29 (4H, m), 7.09-7.17 (2H, m), 7.20-7.24 (1H, m), 7.29-7.42 (5H, m), 7.52-7.64 (1H, m), 8.44-8.52 (1H, m);

MASS (ES+): m/e 553.39 (M+1).

Preparation 172

Compound (172) was obtained in a manner similar to Preparation 16.

MASS (ES+): m/e 786.49 (M+1).

Preparation 173

Compound (172) (crude compound) was purified by flash column chromatography (Silica gel column, eluting with 80 to 100% ethyl acetate/hexane (v/v) then 5% methanol/ethyl acetate (v/v)) to give Compound (173) (1.36 g) as an amorphous solid.

¹H-NMR (300 MHz, CDCl₃, δ): 0.62-0.75 (3H, m), 1.33-2.27 (12H, m), 1.43 (9H, s), 3.02-3.29 (3H, m), 3.41-3.86 (2H, m), 4.00-4.10 (1H, m), 4.27-4.34 (2H, m), 4.40-4.46 (1H, m), 5.10-5.25 (4H, m), 6.96-7.02 (1H, m), 7.05-7.19 (2H, m), 7.28-7.48 (9H, m), 7.50-7.77 (3H, m), 8.00-8.06 (2H, m), 8.44-8.52 (1H, m);

MASS (ES+): m/e 786.41 (M+1).

Preparation 174

Compound (174) was obtained in a manner similar to Preparation 17.

¹H-NMR (300 MHz, CDCl₃, δ): 0.61-0.73 (3H, m), 1.30-2.31 (16H, m), 1.43 (9H, s), 3.08-3.30 (3H, m), 3.35-3.58 (1H, m), 3.78-4.07 (2H, m), 4.23-4.46 (3H, m), 5.11-5.24 (1H, m), 6.90-7.04 (1H, m), 7.13-7.31 (2H, m), 7.37-7.73 (5H, m), 7.99-8.06 (2H, m), 8.45-8.52 (1H, m);

MASS (ES+): m/e 696.49 (M+1).

Preparation 175

Compound (175) was obtained in a manner similar to Preparation 18.

¹H-NMR (300 MHz, CDCl₃, δ): 0.55-2.45 (19H, m), 2.75-3.92 (6H, m), 4.15-4.41 (3H, m), 6.90-6.92 (1H, m), 7.08-7.31 (2H, m), 7.35-7.61 (5H, m), 7.88-8.42 (3H, m), 8.80-8.95 (2H, m);

MASS (ES+): m/e 596.14 (free, M+1).

Preparation 176

Compound (176) was obtained in a manner similar to Preparation 76.

¹H-NMR (300 MHz, CDCl₃, δ): 0.83 (3H, t, J=7.5 Hz), 1.29 (3H, s), 1.33-1.97 (8H, m), 2.02-2.43 (4H, m), 3.12 (1H, dd, J=15.0, 6.0 Hz), 3.52 (1H, dd, J=15.0, 9.0 Hz), 3.75-3.85 (1H, m), 3.87-3.98 (1H, m), 4.20-4.31 (1H, m), 4.31 (2H, t, J=6.8 Hz), 4.64-4.72 (1H, m), 5.58 (1H, dt, J=9.0, 6.0 Hz), 5.87 (1H, s), 7.05-7.30 (4H, m), 7.39-7.62 (4H, m), 8.02 (2H, d, J=7.5 Hz), 8.45 (1H, d, J=4.5 Hz);

MASS (ES+): m/e 578.45 (M+1).

Preparation 177

Compound (177) was obtained in a manner similar to Preparation 77.

¹H-NMR (300 MHz, CDCl₃, δ): 0.84 (1H, t, J=7.2 Hz), 1.21-1.97 (8H, m), 1.29 (3H, s), 2.07-2.45 (4H, m), 3.12 (1H, dd, J=15.3, 6.0 Hz), 3.52 (1H, dd, J=15.3, 10.5 Hz), 3.65 (2H, t, J=6.0 Hz), 3.74-3.84 (1H, m), 3.87-3.98 (1H, m), 4.25 (1H, dt, J=9.9, 7.8 Hz), 4.68 (1H, dd, J=7.8, 2.7 Hz), 5.58 (1H, dt, J=10.5, 5.7 Hz), 5.94-6.03 (1H, m), 7.06-7.13 (1H, m), 7.14-7.24 (2H, m), 7.42-7.64 (2H, m), 8.07-8.13 (1H, m), 8.42-8.48 (1H, m);

MASS (ES+): m/e 474.43 (M+1).

Preparation 178

Compound (178) was obtained in a manner similar to Preparation 78. The obtained compound was used in Example 80.

¹H-NMR (300 MHz, CDCl₃, δ): 0.85 (3H, t, J=7.3 Hz), 1.30 (3H, s), 1.49-2.03 (8H, m), 2.09-2.44 (4H, m), 2.44-2.53 (2H, m), 3.12 (1H, dd, J=15.0, 5.4 Hz), 3.53, (1H, dd, J=15.0, 9.9 Hz), 3.74-3.85 (1H, m), 3.88-3.99 (1H, m), 4.26 (1H, dt, J=10.5, 7.5 Hz), 4.69 (1H, dd, J=7.5, 2.4 Hz), 5.58 (1H, dt, J=9.9, 5.4 Hz), 5.94 (1H, m), 7.07-7.13 (1H, m), 7.15-7.25 (2H, m), 7.42-7.50 (1H, m), 7.57 (1H, dt, J=7.5, 1.8 Hz), 8.43-8.47 (1H, m), 9.77 (1H, t, J=1.5 Hz);

MASS (ES+): m/e 472.44 (M+1).

Preparation 179

Compound (179) was obtained in a manner similar to Preparation 77.

¹H-NMR (300 MHz, CDCl₃, δ): 0.84 (3H, t, J=7.3 Hz), 1.24-1.93 (8H, m), 1.28 (3H, s), 2.06-2.24 (2H, m), 2.16 (3H, s), 2.24-2.41 (2H, m), 2.91 (1H, dd, J=13.6, 5.9 Hz), 3.20 (1H, dd, J=13.6, 9.9 Hz), 3.21-3.33 (1H, m), 3.65 (2H, t, J=6.6 Hz), 3.79-3.90 (1H, m), 4.17-4.29 (1H, m), 4.67 (1H, brd, J=6.0 Hz), 5.15 (1H, dt, J=9.9, 6.2 Hz), 6.00 (1H, s), 7.12 (1H, d, J=9.9 Hz), 7.18 (2H, d, J=8.4 Hz), 7.40 (2H, d, J=8.4 Hz), 7.55 (1H, d, J=10.3 Hz);

MASS (ES+): m/e 530.42 (M+1).

Preparation 180

Compound (180) was obtained in a manner similar to Preparation 78. The obtained compound was used in Example 83.

¹H-NMR (300 MHz, CDCl₃, δ): 0.84 (3H, t, J=7.3 Hz), 1.22-1.33 (1H, m), 1.29 (3H, s), 1.47-1.92 (5H, m), 2.08-2.39 (4H, m), 2.16 (3H, s), 2.50 (2H, brt, J=6.6 Hz), 2.91 (1H, dd, J=13.6, 5.9 Hz), 3.18-3.33 (1H, m), 3.20 (1H, dd, J=13.6, 9.9 Hz), 3.80-3.91 (1H, m), 4.16-4.30 (1H, m), 4.66 (1H, brd, J=6.7 Hz), 5.15 (1H, dt, J=10.1, 5.9 Hz), 5.90 (1H, s), 7.13 (1H, d, J=7.3 Hz), 7.15 (1H, s), 7.18 (2H, d, J=8.4 Hz), 7.40 (2H, d, J=8.4 Hz), 7.49 (1H, d, J=10.6 Hz);

MASS (ES+): m/e 528.32 (M+1).

Preparation 181

Compound (181) was obtained in a manner similar to Preparation 13.

¹H-NMR (300 MHz, CDCl₃, δ): 1.07-1.31 (2H, m), 1.35 (4.5H, brs), 1.45 (4.5H, brs), 1.50-1.75 (3H, m), 2.10-2.32 (1H, m), 2.74-3.05 (1H, m), 3.81-4.10 (1H, m), 4.75 (0.5H, brs), 4.95 (0.5H, brs), 5.05-5.25 (2H, m), 7.25-7.40 (5H, m);

MASS (ES+): m/e 320.29 (M+1).

Preparation 182

Compound (182) was obtained in a manner similar to Preparation 14.

¹H-NMR (300 MHz, CDCl₃, δ): 0.49-0.69 (1H, m), 1.05-1.29 (1H, m), 1.42 (9H, s), 1.30-1.77 (3H, m), 2.14-2.25 (1H, m), 2.89-3.19 (3H, m), 3.48-3.62 (1H, m), 4.84-5.01 (1H, m), 5.08-5.23 (2H, m), 5.25-5.33 (1H, m), 5.43 (1H, brd, J=8.1 Hz), 7.02-7.40 (10H, m);

MASS (ES+): m/e 467.41 (M+1).

Preparation 183

Compound (183) was obtained in a manner similar to Preparation 15.

¹H-NMR (300 MHz, CDCl₃, δ): 0.54-0.72 (1H, m), 0.78 (2.1H, t, J=7.3 Hz), 0.99 (0.9H, m, J=7.3 Hz), 1.07-1.25 (1H, m), 1.31-2.03 (5H, m), 1.40 (3H, s), 1.42 (9H, s), 2.15-2.26 (1H, m), 2.66-3.20 (3H, m), 3.51-3.60 (1H, m), 4.98-5.30 (3H, m), 6.87-6.96 (0.7H, m), 7.02-7.10 (0.3H, m), 7.13-7.40 (11H, m);

MASS (ES+): m/e 566.46 (M+1).

Preparation 184

Compound (184) was obtained in a manner similar to Preparation 16.

¹H-NMR (300 MHz, CDCl₃, δ): 0.60-0.85 (1H, m), 0.70 (3H, t, J=7.3 Hz), 1.08-1.30 (2H, m), 1.33-2.00 (9H, m), 1.44 (12H, s), 2.18-2.41 (2H, m), 2.92-3.21 (3H, m), 3.56-3.68 (1H, m), 3.95-4.16 (1H, m), 4.32 (2H, t, J=6.6 Hz), 5.00-5.31 (4H, m), 6.79 (1H, brd, J=8.1 Hz), 6.99-7.08 (1H, m), 7.14-7.39 (6H, m), 7.40-7.48 (2H, m), 7.51-7.62 (1H, m), 8.00-8.08 (2H, m);

MASS (ES+): m/e 799.47 (M+1).

Preparation 185

Compound (185) was obtained in a manner similar to Preparation 17.

¹H-NMR (300 MHz, CDCl₃, δ): 0.59-0.78 (1H, m), 0.76 (3H, t, J=7.3 Hz), 1.17-2.07 (13H, m), 1.40 (3H, s), 1.43 (9H, s), 2.19-2.30 (1H, m), 2.86-3.20 (3H, m), 3.62-3.77 (1H, m), 3.96-4.09 (1H, m), 4.25-4.39 (2H, m), 5.13-5.25 (2H, m), 5.43 (1H, brs), 6.96 (1H, brs), 7.11-7.35 (6H, m), 7.39-7.49 (2H, m), 7.52-7.62 (1H, m), 8.00-8.08 (2H, m);

MASS (ES+): m/e 709.48 (M+1).

Preparation 186

Compound (186) was obtained in a manner similar to Preparation 18.

¹H-NMR (300 MHz, CDCl₃, δ): 0.50-0.91 (4H, m), 1.03-2.23 (13H, m), 1.40 (3H, brs), 2.82-3.34 (3H, m), 3.42-3.66 (1H, m), 3.70-4.10 (1H, m), 4.19-4.52 (2H, m), 4.60-4.86 (1H, m), 5.05-5.28 (1H, m), 7.07-7.32 (5H, m), 7.34-7.47 (2H, m), 7.48-7.59 (1H, m), 7.83-8.17 (2H, m);

MASS (ES+): m/e 609.44 (free, M+1).

Preparation 187

Compound (187) was obtained in a manner similar to Preparation 76.

¹H-NMR (300 MHz, CDCl₃, δ): 0.75 (3H, t, J=7.3 Hz), 1.20-2.16 (13H, m), 2.19-2.31 (1H, m), 2.93 (1H, dt, J=13.4, 2.6 Hz), 3.04 (1H, dd, J=13.9, 7.3 Hz), 3.21 (1H, dd, J=13.9, 8.1 Hz), 3.94-4.05 (1H, m), 4.19-4.32 (1H, m), 4.31 (2H, t, J=6.2 Hz), 5.00-5.07 (1H, m), 5.36 (1H, dt, J=10.3, 7.7 Hz), 6.05 (1H, s), 6.53 (1H, d, J=10.6 Hz), 7.16-7.32 (5H, m), 7.39-7.48 (2H, m), 7.49-7.60 (2H, m), 7.98-8.06 (2H, m);

MASS (ES+): m/e 591.49 (M+1).

Preparation 188

Compound (188) was obtained in a manner similar to Preparation 77.

¹H-NMR (300 MHz, CDCl₃, δ): 0.78 (3H, t, J=7.3 Hz), 1.18-2.34 (14H, m), 1.27 (3H, s), 2.93 (1H, dt, J=13.2, 2.6 Hz), 3.04 (1H, dd, J=13.9, 7.3 Hz), 3.21 (1H, dd, J=13.9, 7.7 Hz), 3.59-3.71 (2H, m), 4.00 (1H, brd, J=13.6 Hz), 4.20-4.32 (1H, m), 5.04 (1H, brd, J=6.2 Hz), 5.36 (1H, dt, J=10.3, 7.7 Hz), 6.16 (1H, s), 6.54 (1H, d, J=10.3 Hz), 7.15-7.32 (5H, m), 7.54 (1H, d, J=9.9 Hz);

MASS (ES+): m/e 487.40 (M+1).

Preparation 189

Compound (189) was obtained in a manner similar to Preparation 78. The obtained compound was used in Example 86.

¹H-NMR (300 MHz, CDCl₃, δ): 0.78 (3H, t, J=7.3 Hz), 1.18-1.37 (1H, m), 1.29 (3H, s), 1.45-2.31 (1H, m), 2.47-2.56 (2H, m), 2.94 (1H, dt, J=13.5, 2.9 Hz), 3.04 (1H, dd, J=13.9, 7.3 Hz), 3.21 (1H, dd, J=13.9, 7.7 Hz), 3.98 (1H, brd, J=13.2 Hz), 4.18-4.31 (1H, m), 5.04 (1H, brd, J=6.2 Hz), 5.36 (1H, dt, J=9.7, 7.9 Hz), 5.98 (1H, s), 6.50 (1H, d, J=10.6 Hz), 7.15-7.32 (5H, m), 7.43 (1H, d, J=9.9 Hz), 9.76-9.79 (1H, m);

MASS (ES+): m/e 485.33 (M+1).

Preparation 190

Compound (190) was obtained in a manner similar to Preparation 77.

¹H-NMR (300 MHz, CDCl₃, δ): 0.82 (3H, t, J=7.3 Hz), 1.22-1.72 (6H, m), 1.28 (3H, s), 1.74-1.93 (2H, m), 2.08-2.41 (4H, m), 2.96 (1H, dd, J=13.9, 6.6 Hz), 2.99 (3H, s), 3.20 (1H, dd, J=13.9, 9.2 Hz), 3.25-3.37 (1H, m), 3.65 (2H, t, J=6.4 Hz), 3.79-3.91 (1H, m), 4.24 (1H, dt, J=10.3, 7.5 Hz), 4.70 (1H, brd, J=7.7 Hz), 5.15 (1H, dt, J=9.7, 6.4 Hz), 6.07 (1H, s), 6.65 (1H, brs), 7.10 (1H, d, J=9.3 Hz), 7.13 (2H, d, J=8.4 Hz), 7.22 (2H, d, J=8.4 Hz), 7.61 (1H, d, J=10.3 Hz);

MASS (ES+): m/e 566.40 (M+1).

Preparation 191

Compound (191) was obtained in a manner similar to Preparation 78. The obtained compound was used in Example 90.

¹H-NMR (300 MHz, CDCl₃, δ): 0.83 (3H, t, J=7.3 Hz), 1.29 (3H, s), 1.51-1.91 (4H, m), 2.08-2.39 (6H, m), 2.50 (2H, brt, J=7.3 Hz), 2.91-2.99 (1H, m), 2.99 (3H, S), 3.20 (1H, dd, J=13.9, 9.2 HZ), 3.25-3.36 (1H, m), 3.80-3.91 (1H, m), 4.18-4.30 (1H, m), 4.69 (1H, brd, J=7.3 HZ), 5.09-5.21 (1H, m), 6.01 (1H, s), 6.59 (1H, S), 7.07-7.17 (3H, m), 7.22 (2H, d, J=8.4 Hz), 7.55 (1H, d, J=10.3 Hz);

MASS (ES+): m/e 564.41 (M+1).

Preparation 192

Compound (192) was obtained in a manner similar to Preparation 14.

¹H-NMR (300 MHz, CDCl₃, δ): 1.43 (9H, m), 1.46-1.59 (1H, m), 1.72-2.02 (3H, m), 2.69-2.84 (1H, m), 2.98 (1H, dd, J=13.0, 8.8 Hz), 3.10 (1H, dd, J=13.0, 5.5 Hz), 3.49-3.67 (1H, m), 4.38 (1H, dd, J=8.1, 3.7 Hz), 4.68 (1H, dt, J=8.8, 5.5 Hz), 4.99-5.24 (2H, m), 5.40 (1H, d, J=8.8 Hz), 7.23-7.60 (14H, m);

MASS (ES+): m/e 529.38 (M+1).

Preparation 193

Compound (193) was obtained in a manner similar to Preparation 15.

¹H-NMR (300 MHz, CDCl₃, δ): 0.81 (3H, t, J=7.4 Hz), 1.38 (1.5H, S), 1.41 (1.5H, S), 1.44 (9H, S), 1.70-2.09 (4H, m), 2.74-2.95 (1H, m), 2.99 (1H, dd, J=13.3, 9.6 Hz), 3.13 (1H, dd, J=13.3, 5.1 Hz), 3.51-3.66 (1H, m), 4.39 (1H, dd, J=7.6, 3.3 Hz), 4.93-5.04 (1H, m), 5.06-5.26 (2H, m), 6.90 (1H, d, J=7.6 Hz), 7.27-7.59 (14H, m);

MASS (ES+): m/e 628.50.

Preparation 194

Compound (194) was obtained in a manner similar to Preparation 16.

¹H-NMR (300 MHz, CDCl₃, δ): 0.58 (0.6H, t, J=7.3 Hz), 0.73 (2.4H, t, J=7.3 Hz), 1.42 (3H, S), 1.44 (9H, S), 1.48-2.03 (9H, m), 2.83-2.96 (1H, m), 2.99-3.14 (2H, m), 3.54-3.66 (1H, m), 3.93-4.15 (1H, m), 4.25-4.36 (2H, m), 4.40 (1H, dd, J=7.6, 3.3 Hz), 4.92-5.03 (1H, m), 5.06-5.21 (2H, m), 6.72-6.90 (1H, m), 6.98 (1H, S), 7.23-7.60 (19H, m), 7.99-8.06 (2H, m);

MASS (ES+): m/e 861.60 (M+1).

Preparation 195

Compound (195) was obtained in a manner similar to Preparation 17.

¹H-NMR (300 MHz, CDCl₃, δ): 0.77 (3H, t, J=7.3 Hz), 1.44 (12H, s), 1.46-2.21 (12H, m), 2.81-2.94 (1H, m), 3.00-3.11 (2H, m), 3.65-3.77 (1H, m), 3.96-4.10 (1H, m), 4.23-4.42 (3H, m), 4.97 (1H, q, J=8.1 Hz), 6.84 (1H, brs), 7.22-7.62 (13H, m), 7.98-8.07 (2H, m);

MASS (ES+): m/e 771.52 (M+1).

Preparation 196

Compound (196) was obtained in a manner similar to Preparation 18.

¹H-NMR (300 MHz, CDCl₃, δ): 0.70 (3H, brt, J=7.3 Hz), 1.39 (3H, s), 1.54-2.21 (12H, m), 2.86-3.39 (3H, m), 3.67-3.82 (1H, m), 4.18-4.38 (4H, m), 4.91-5.05 (1H, m), 7.23-7.54 (12H, m), 7.72 (1H, brd, J=8.8 Hz), 7.99 (2H, d, J=7.0 Hz), 8.22 (2H, brs), 8.42 (1H, brs);

MASS (ES+): m/e 671.53 (free, M+1).

Preparation 197

Compound (197) was obtained in a manner similar to Preparation 76.

¹H-NMR (300 MHz, CDCl₃, δ): 0.82 (3H, t, J=7.3 Hz), 1.28 (3H, s), 1.35-1.57 (2H, m), 1.64-2.00 (6H, m), 2.07-2.41 (4H, m), 3.01 (1H, dd, J=13.5, 6.3 Hz), 3.21-3.38 (2H, m), 3.81-3.95 (1H, m), 4.19-4.31 (1H, m), 4.32 (2H, t, J=6.4 Hz), 4.69 (1H, brd, J=5.9 Hz), 5.16-5.29 (1H, m), 5.93 (1H, s), 7.15 (1H, d, J=10.3 Hz), 7.27-7.36 (4H, m), 7.38-7.47 (4H, m), 7.48-7.63 (5H, m), 8.03 (2H, d, J=7.3 Hz);

MASS (ES+): m/e 653.45 (M+1).

Preparation 198

Compound (198) was obtained in a manner similar to Preparation 77.

¹H-NMR (300 MHz, CDCl₃, δ): 0.85 (3H, t, J=7.3 Hz), 1.29 (3H, s), 1.30-1.95 (8H, m), 2.07-2.41 (4H, m), 3.01 (1H, dd, J=13.6, 6.3 Hz), 3.20-3.38 (2H, m), 3.66 (2H, t, J=6.3 Hz), 3.82-3.95 (1H, m), 4.18-4.31 (1H, m), 4.70 (1H, brd, J=5.9 Hz), 5.16-5.29 (1H, m), 5.97 (1H, s), 7.14 (1H, d, J=10.6 Hz), 7.24-7.65 (9H, m);

MASS (ES+): m/e 549.47 (M+1).

Preparation 199

Compound (199) was obtained in a manner similar to Preparation 78. The obtained compound was used in Example 93.

¹H-NMR (300 MHz, CDCl₃, δ): 0.84 (3H, t, J=7.3 Hz), 1.30 (3H, s), 1.52-1.94 (6H, m), 2.09-2.40 (4H, m), 2.51 (2H, brt, J=6.2 Hz), 3.01 (1H, dd, J=13.5, 6.2 Hz), 3.21-3.38 (2H, m), 3.83-3.95 (1H, m), 4.18-4.31 (1H, m), 4.69 (1H, brd, J=5.4 Hz), 5.16-5.29 (1H, m), 5.88 (1H, S), 7.14 (1H, d, J=10.2 Hz), 7.24-7.37 (3H, m), 7.38-7.47 (2H, m), 7.48-7.60 (5H, m), 9.78 (1H, S);

MASS (ES+): m/e 547.44 (M+1).

Preparation 200

Compound (200) was obtained in a manner similar to Preparation 14.

¹H-NMR (300 MHz, CDCl₃, δ): 1.44 (3×3H, S), 1.53 (1H, m), 1.75-2.00 (3H, m), 2.65 (1H, m), 2.88 (1H, dd, J=13, 10 Hz), 3.02 (1H, dd, J=13, 6 Hz), 3.53 (1H, m), 3.85 (2×3H, S), 4.36 (1H, dd, J=8, 4 Hz), 4.62 (1H, ddd, J=10, 8, 6 Hz), 5.11 (1H, d, J=12 Hz), 5.21 (1H, d, J=12 Hz), 5.38 (1H, d, J=8 Hz), 6.70-6.79 (3H, m), 7.28-7.40 (5H, m);

MASS (ES+): m/e 513.

Preparation 201

Compound (201) was obtained in a manner similar to Preparation 21.

¹H-NMR (300 MHz, CDCl₃, δ): 1.48 (1H, m), 1.70-1.90 (3H, m), 2.50 (1H, m), 3.11 (1H, m), 3.57 (1H, m), 3.72 (1H, m), 3.81 (3H, s), 3.84 (3H, S), 4.35 (1H, m), 4.66 (1H, m), 5.04 (1H, d, J=12 Hz), 5.13 (1H, d, J=12 Hz), 6.66-6.96 (3H, m), 7.22-7.37 (5H, m);

MASS (ES+): m/e 413.

Preparation 202

Compound (202) was obtained in a manner similar to Preparation 22.

¹H-NMR (300 MHz, CDCl₃, δ): 0.60 (3× 1/7H, t, J=7.5 Hz), 0.81 (3× 6/7H, t, J=7.5 Hz), 1.32 (3× 1/7H, S), 1.39 (3×3× 1/7H, S), 1.41 (3× 6/7H, S), 1.43 (3×3× 6/7H, S), 1.50-1.70 (2H, m), 1.76-2.02 (4H, m), 2.68 (1H, m), 2.88 (1H, dd, J=13.5, 9.5 Hz), 3.02 (1H, dd, J=13.5, 5 Hz), 3.56 (1H, m), 3.81 (3× 1/7H, S), 3.82 (3× 1/7H, S), 3.84 (3× 6/7H, S), 3.85 (3× 6/7H, S), 4.38 (1H, dd, J=8, 4 Hz), 4.92 (1H, ddd, J=9.5, 8.5 Hz), 5.11 (1H, d, J=12.5 Hz), 5.13 (1H, br), 5.15 (1H, d, J=12.5 Hz), 6.59-6.79 (3H, m), 6.88 (1H, d, J=8 Hz), 7.28-7.40 (5H, m);

MASS (ES+): m/e 612.

Preparation 203

Compound (203) was obtained in a manner similar to Preparation 23.

¹H-NMR (300 MHz, CDCl₃, δ): 0.46 (3×⅓H, t, J=7.5 Hz), 0.89 (3×⅔H, t, J=7.5 Hz), 1.40-2.33 (6H, m), 1.50 (3×⅓H, s), 1.66 (3×⅔H, s), 2.85 (1×⅔H, m), 2.93-3.18 (2H, m), 3.50-3.90 (1+⅓H, m), 3.81 (3×⅓H, s), 3.83 (3×⅔H, s), 3.84 (3×⅔H, s), 3.85 (3×⅓H, s), 4.33 (1×⅔H, m), 4.67 (1/1/3H, m), 4.94 (1×⅔H, m), 5.07-5.34 (2+⅓H, m), 6.65-7.06 (3H, m), 7.23-7.41 (5H, m), 7.67 (1×⅔H, J=8 Hz), 8.43 (1×⅓H, d, J=8 Hz);

MASS (ES+): m/e 512.

Preparation 204

Compound (204) was obtained in a manner similar to Preparation 24.

¹H-NMR (300 MHz, CDCl₃, δ): 0.63 (3×⅛H, t, J=7.5 Hz), 0.74 (3×⅞H, t, J=7.5 Hz), 1.35 (3×⅛H, s), 1.42 (3×3×⅛H, s), 1.44 (3×3×⅞H, s9, 1.50 (3×⅞H, s), 2.76 (1H, m), 2.92 (1H, dd, J=13.5, 9 Hz), 2.98 (1H, dd, J=13.5, 5 Hz), 3.57 (1H, m), 3.81 (2×3×⅛H, s), 3.84 (2×3×⅞H, s), 4.07 (1H, m), 4.32 (2H, t, J=6.5 Hz), 4.38 (1H, dd, J=8.4 Hz), 4.91 (1H, m), 5.13 (2H, s), 5.13 (1H, br), 6.59-6.83 (4H, m), 6.97 (1H, s), 7.28-7.40 (5H, m), 7.42 (2×1H, dd, J=7.5, 7.5 Hz);

MASS (ES+): m/e 845.

Preparation 205

Compound (205) was obtained in a manner similar to Preparation 25.

¹H-NMR (300 MHz, CDCl₃, δ): 0.78 (3H, t, J=7.5 Hz), 1.36-2.24 (12H, m), 1.44 (3×4H, s), 2.78 (1H, m), 2.97 (2H, d, J=7 Hz), 3.67 (1H, m), 3.80 (2×3H, s), 4.27-4.41 (3H, m), 4.91 (1H, dt, J=7.5, 7 Hz), 5.23 (1H, br), 6.71-6.80 (3H, m), 6.83 (1H, s), 7.28 (1H, d, J=7.5 Hz), 7.43 (2×1H, dd, J=7.5, 7.5 Hz), 7.56 (1H, dd, J=7.5, 7.5 Hz), 8.03 (2×1H, d, J=7.5 Hz);

MASS (ES+): m/e 753.

Preparation 206

Compound (206) was obtained in a manner similar to Preparation 18.

¹H-NMR (300 MHz, CDCl₃, δ): 0.73 (3H, brt, J=7 Hz), 1.40 (3H, s), 1.54-2.17 (12H, m), 2.80-3.08 (3H, m), 3.76 (1H, m), 3.81 (3H, s), 3.83 (3H, s), 4.20-4.40 (4H, m), 4.92 (1H, m), 6.68-6.82 (3H, m), 7.40 (2×1H, dd, J=7.5, 7.5 Hz), 7.53 (1H, dd, J=7.5, 7.5 Hz), 7.66 (1H, brd, J=7 Hz), 8.00 (2×1H, d, J=7.5 Hz), 8.21 (2H, br), 8.36 (1H, br);

MASS (ES−): m/e 653.

Preparation 207

Compound (207) was obtained in a manner similar to Preparation 76.

¹H-NMR (300 MHz, CDCl₃, δ): 0.83 (3H, t, J=7.3 Hz), 1.28 (3H, s), 1.47 (2H, m), 1.56-2.00 (6H, m), 2.06-2.40 (4H, m), 2.90 (1H, dd, J=13.5, 6 Hz), 3.20 (1H, dd, J=13.5, 10 Hz), 3.26 (1H, m), 3.85 (2×3H, s), 3.86 (1H, m), 4.24 (1H, dt, J=10, 7.5 Hz), 4.32 (2H, t, J=6.5 Hz), 4.67 (1H, m), 5.16 (1H, ddd, J=10, 10, 6 Hz), 5.88 (1H, s), 6.75-6.80 (3H, m), 7.14 (1H, d, J=10 Hz), 7.44 (2×1H, dd, J=7.5, 7.5 Hz), 7.52-7.60 (2H, m), 8.03 (2×1H, dd, J=7.5, 1.5 Hz);

MASS (ES+): m/e 637.

Preparation 208

Compound (208) was obtained in a manner similar to Preparation 76.

¹H-NMR (300 MHz, CDCl₃, δ): 0.82 (3H, t, J=7.3 Hz), 1.28 (3H, s), 1, 46, (2H, m), 1.62-2.06 (6H, m), 2.08-2.40 (4H, m), 2.90 (1H, dd, J=13.5, 6 Hz), 3.08-3.33 (2H, m), 3.85 (2×3H, s), 3.85 (1H, m), 4.24 (1H, m), 4.32 (1H, t, J=6.5 Hz), 4.67 (1H, m), 5.15 (1H, m), 5.91 (1H, s), 6.74-6.80 (3H, m), 7.15 (1H, d, J=10 Hz), 7.44 (2×1H, dd, J=7.5, 7.5 Hz), 7.52-7.62 (2H, m), 8.03 (2×1H, dd, J=7.5, 1.5 Hz);

MASS (ES−): m/e 635.

Preparation 20.9

Compound (209) was obtained in a manner similar to Preparation 77.

¹H-NMR (300 MHz, CDCl₃, δ): 0.84 (3H, t, J=7.3 Hz), 1.29 (3H, s), 1.30-1.53 (2H, m), 1.54-1.94 (6H, m), 2.07-2.40 (4H, m), 2.90 (1H, dd, J=13.5, 6 Hz), 3.19 (1H, dd, J=13.5, 10 Hz), 3.26 (1H, m), 3.66 (2H, t, J=6.5 Hz), 3.85 (2×3H, s), 3.85 (1H, m), 4.22 (1H, dt, J=10, 7.5 Hz), 4.67 (1H, m), 5.15 (1H, ddd, J=10, 10, 6 Hz), 5.97 (1H, s), 6.74-6.80 (3H, m), 7.14 (1H, d, J=10 Hz), 7.55 (1H, d, J=10 Hz);

MASS (ES−): m/e 531.

Preparation 210

Compound (210) was obtained in a manner similar to Preparation 77.

¹H-NMR (300 MHz, CDCl₃, δ): 0.85 (3H, t, J=7.5 Hz), 1.24-1.51 (2H, m), 1.29 (3H, s), 1.53-1.93 (6H, m), 2.08-2.40 (4H, m), 2.90 (1H, dd, J=13.5, 6 Hz), 3.19 (1H, dd, J=13.5, 10 Hz), 3.26 (1H, m), 3.66 (2H, t, J=6.5 Hz), 3.85 (2×3H, s), 3.86 (1H, m), 4.23 (1H, dt, J=10, 7.5 Hz), 4.68 (1H, m), 5.16 (1H, ddd, J=10, 10, 6 Hz), 6.03 (1H, s), 6.74-6.80 (3H, m), 7.15 (1H, d, J=10 Hz), 7.56 (1H, d, J=10 Hz);

MASS (ES−): m/e 531.

Preparation 211

Compound (211) was obtained in a manner similar to Preparation 78. The obtained compound was used in Example 96.

¹H-NMR (300 MHz, CDCl₃, δ): 0.85 (3H, t, J=7.3 Hz), 1.30 (3H, s), 1.50-1.92 (6H, m), 2.08-2.40 (4H, m), 2.50 (2H, m), 2.90 (1H, dd, J=13.5, 6 Hz), 3.19 (1H, dd, J=13.5, 9.5 Hz), 3.25 (1H, m), 3.85 (2×3H, s), 3.86 (1H, m), 4.23 (1H, dt, J=10, 7.3 Hz), 4.67 (1H, dd, J=8, 2.5 Hz), 5.15 (1H, ddd, J=10, 9.5, 6 Hz), 5.93 (1H, s), 6.73-6.80 (3H, m), 7.16 (1H, d, J=10 Hz), 7.50 (1H, d, J=10 Hz), 9, 77 (1H, t, J=1 Hz);

MASS (ES−): m/e 529.

Preparation 212

Compound (212) was obtained in a manner similar to Preparation 14.

¹H-NMR (300 MHz, CDCl₃, δ): 0.74 (3× 1/7H, d, J=7 Hz), 0.80 (3× 1/7H, t, J=7 Hz), 0.89 (3× 6/7H, t, J=7 Hz), 0.94 (3× 6/7H, d, J=7 Hz), 1.12 (1H, m), 1.38-1.80 (3H, m), 1.42 (9× 1/7H, s), 1.44 (9× 6/7H, s), 1.88-2.26 (3H, m), 3.57 (1H, m), 3.90 (1H, m), 4.36 (1H, dd, J=9, 7 Hz), 4.49 (1H, dd, J=8, 3 Hz), 5.13 (1H, d, J=12.5 Hz), 5.19 (1H, d, J=9 Hz), 5.20 (1H, d, J=12.5 Hz), 7.28-7.41 (5H, m);

MASS (ES+): m/e 419.

Preparation 213

Compound (213) was obtained in a manner similar to Preparation 21.

¹H-NMR (300 MHz, CDCl₃, δ): 0.70 (3× 1/7H, t, J=7.3 Hz), 0.75 (3× 1/7H, d, J=7 Hz), 0.86 (3× 6/7H, t, J=7.3 Hz), 0.98 (3× 6/7H, d, J=7 Hz), 1.13 (1H, m), 1.43 (1H, m), 1.76-2.02 (4H, m), 2.18 (1H, m), 3.52 (1H, m), 3.79 (1H, m), 4.13 (1H, m), 4.41 (1H, m), 5.10 (1× 6/7H, d, J=12.5 Hz), 5.12 (1× 1/7H, d, J=12.5 Hz), 5.19 (1× 6/7H, d, J=12.5 Hz), 5.22 (1× 1/7H, d, J=12.5 Hz), 7.30-7.44 (5H, m), 8.20 (2× 6/7H, br), 8.32 (2× 1/7H, br);

MASS (ES+): m/e 319.

Preparation 214

Compound (214) was obtained in a manner similar to Preparation 22.

¹H-NMR (300 MHz, CDCl₃, δ): 0.74 (3×⅕H, d, J=6.5 Hz), 0.79 (3H, t, J=7.5 Hz), 0.80 (3×⅕H, t, J=7.5 Hz), 0.87 (3×⅘H, t, J=7.5 Hz), 0.94 (3×⅘H, d, J=6.5 Hz), 1.11 (1H, m), 1.39 (3H, s), 1.41 (9×⅕H, s), 1.43 (9×⅘H, s), 1.52-2.24 (8H, m), 3.57 (1H, m), 3.92 (1H, m), 4.32 (1×⅕H, dd, J=9.5, 7.5 Hz), 4.49 (1H, dd, J=8, 3 Hz), 4.68 (1H, dd, J=9.5, 7.5 Hz), 5.13 (2H, s), 5.14 (1H, br), 6.58 (1×⅕H, d, J=9.5 Hz), 6.67 (1×⅘H, d, J=9.5 Hz), 7.24-7.40 (5H, m);

MASS (ES+): m/e 518.

Preparation 215

Compound (215) was obtained in a manner similar to Preparation 23.

¹H-NMR (300 MHz, CDCl₃, δ): 0.71 (3×⅓H, d, J=6.5 Hz), 0.77 (3×⅓H, t, J=7 Hz), 0.82-0.99 (7H, m), 0.99-2.22 (9H, m), 1.63 (3×⅓H, s), 1.69 (3×⅔H, s), 3.56 (1H, m), 4.04 (1H, m), 4.28 (1×⅓H, dd, J=9.8 Hz), 4.46 (1H, dd, J=8.3 Hz), 4.63 (1×⅔H, dd, J=9, 8 Hz), 5.09-5.27 (2H, m), 7.25-7.40 (5H, m), 7.49 (1×⅔H, d, J=8 Hz), 8.05 (1×⅓H, d, J=8 Hz);

MASS (ES+): m/e 418.

Preparation 216

Compound (216) was obtained in a manner similar to Preparation 24.

¹H-NMR (300 MHz, CDCl₃, δ): 0.66-0.97 (9×⅕H, m), 0.72 (3×⅘H, t, J=7.3 Hz), 0.87 (3×⅘H, t, J=7.4 Hz), 0.93 (3×⅘H, d, J=6.7 Hz), 1.00-2.45 (15H, m), 1.43 (3×3H, s), 1.50° (3×⅕H, s), 1.54 (3×⅘H, s), 3.57 (1H, m), 3.90 (1H, m), 4.08 (1H, m), 4.25-4.36 (2H, m), 4.59 (1H, dd, J=8, 3 Hz), 4.68 (1H, dd, J=9, 8 Hz), 5.02-5.24 (3H, m), 6.54 (1H, d, J=9 Hz), 6.91 (1×⅕H, s), 7.07 (1×⅘H, s), 7.27-7.47 (7H, m), 7.55 (1H, m), 8.02 (2×1H, d, J=7 Hz);

MASS (ES+): m/e 751.

Preparation 217

Compound (217) was obtained in a manner similar to Preparation 17.

¹H-NMR (300 MHz, CDCl₃, δ): 0.80 (3H, t, J=7.4 Hz), 0.88 (3H, t, J=7.3 Hz), 0.91 (3H, d, J=7.0 Hz), 1.04-2.38 (15H, m), 3.56 (1H, m), 3.92-4.12 (2H, m), 4.26-4.38 (2H, m), 4.50 (1H, m), 4.60 (1H, dd, J=9, 8 Hz), 5.28 (1H, br), 6.96 (1H, brs), 7.15 (1H, brd, J=9 Hz), 7.43 (2×1H, dd, J=7.5, 7.5 Hz), 7.56 (1H, m), 8.03 (2×1H, dd, J=7.5, 1.5 Hz);

MASS (ES−): m/e 659.

Preparation 218

Compound (218) was obtained in a manner similar to Preparation 18.

¹H-NMR (300 MHz, CDCl₃, δ): 0.78-0.94 (9H, m), 1.02-2.22 (15H, m), 1.42 (3H, s), 3.52 (1H, m), 3.96 (1H, m), 4.20-4.40 (4H, m), 4.56 (1H, dd, J=9.8 Hz), 7.35-7.57 (4H, m), 8.01 (2×1H, d, J=7.5 Hz), 8.13 (2H, br), 8.36 (1H, brs);

MASS (ES+): m/e 561.

Preparation 219

Compound (219) was obtained in a manner similar to Preparation 76.

¹H-NMR (300 MHz, CDCl₃, δ): 0.86 (3H, t, J=7 Hz), 0.87 (3H, d, J=7 Hz), 0.91 (3H, t, J=7 Hz), 1.17 (2H, m), 1.29 (3H, s), 1.34-2.10 (9H, m), 2.11-2.42 (4H, m), 3.52 (1H, dt, J=10, 7.5 Hz), 3.89 (1H, ddd, J=10, 8.5, 5 Hz), 4.24 (1H, dt, J=10.5, 7.5 Hz), 4.31 (2H, t, J=7 Hz), 4.56 (1H, dd, J=10.5, 10.5 Hz), 4.77 (1H, dd, J=8, 2 Hz), 5.86 (1H, s), 7.19 (1H, d, J=10.5 Hz), 7.37 (1H, d, J=10.5 Hz), 7.43 (2×1H, dd, J=7.5, 7.5 Hz), 7.56 (1H, m), 8.02 (2×1H, dd, J=7.5, 1 Hz);

MASS (ES+): m/e 543.

Preparation 220

Compound (220) was obtained in a manner similar to Preparation 77.

¹H-NMR (300 MHz, CDCl₃, δ): 0.86 (3H, d, J=7 Hz), 0.88 (3H, t, J=7 Hz), 0.91 (3H, t, J=7 Hz), 1.08-1.51 (4H, m), 1.30 (3×3H, s), 1.53-1.76 (3H, m), 1.77-2.11 (4H, m), 2.13-2.43 (4H, m), 3.52 (1H, dt, J=10, 7.5 Hz), 3.65 (2H, t, J=7 Hz), 3.89 (1H, ddd, J=10, 8.5, 5 Hz), 4.23 (1H, dt, J=10, 7.5 Hz), 4.58 (1H, dd, J=10.5, 10.5 Hz), 4.76 (1H, dd, J=7.5, 2 Hz), 6.01 (1H, s), 7.20 (1H, d, J=10 Hz), 7.38 (1H, d, J=10.5 Hz);

MASS (ES−): m/e 437.

Preparation 221

Compound (221) was obtained in a manner similar to Preparation 78. The obtained compound was used in Example 99.

¹H-NMR (300 MHz, CDCl₃, δ): 0.86 (3H, d, J=7 Hz), 0.88 (3H, t, J=7 Hz), 0.91 (3H, t, J=7 Hz), 1.17 (2H, m), 1.31 (3H, S), 1.50-1.75 (3H, m), 1.74-2.10 (4H, m), 2.14-2.44 (4H, m), 2.49 (2H, m), 3.52 (1H, dt, J=10, 7.5 Hz), 3.89 (1H, ddd, J=10, 8.5, 4.5 Hz), 4.23 (1H, dt, J=10, 7 Hz), 4.58 (1H, dd, J=10.5, 10.5 Hz), 4.78 (1H, dd, J=8, 2 Hz), 5.91 (1H, S), 7.20 (1H, d, J=10 Hz), 7.31 (1H, d, J=10 Hz), 9.77 (1H, t, J=1 Hz);

MASS (ES−): m/e 435.

Preparation 222

Compound (222) was obtained in a manner similar to Preparation 22.

¹H-NMR (300 MHz, CDCl₃, δ): 0.69 (3×⅕H, t, J=7 Hz), 0.71 (3×⅕H, d, J=7 Hz), 0.81 (3×⅘H, t, J=7 Hz), 0.87 (3×⅘H, d, J=7 Hz), 1.32-1.78 (4H, m), 1.39 (3×3H, S), 1.88-2.26 (3H, m), 2.82-3.10 (2H, m), 3.56 (1H, m), 3.77 (3×⅘H, S), 3.80 (3×⅕H, S), 3.92 (1H, m), 4.35 (1H, m), 4.48 (1H, dd, J=8, 3 Hz), 4.67 (1H, dd, J=9, 7 Hz), 4.94 (1H, m), 5.13 (1×⅘H, d, J=12.5 Hz), 5.15 (1×⅕H, d, J=12 Hz), 5.19 (1×⅘H, d, J=12.5 Hz), 5.21 (1×⅕H, d, J=12 Hz), 6.56 (1H, brd, J=9 Hz), 6.81 (2×⅕H, d, J=8.5 Hz), 6.84 (2×⅘H, d, J=8.5 Hz), 7.06 (2×⅕H, d, J=8.5 Hz), 7.10 (2×⅘H, d, J=8.5 Hz), 7.29-7.42 (5H, m);

MASS (ES+): m/e 596.

Preparation 223

Compound (223) was obtained in a manner similar to Preparation 23.

¹H-NMR (300 MHz, CDCl₃, δ): 0.49-0.60 (2H, m), 0.69-0.79 (4H, m), 0.79-0.98 (2H, m), 1.25 (1H, m), 1.66 (1H, m), 1.76-2.00 (2H, m), 2.17 (1H, m), 2.82-2.96 (1+⅓H, m), 3.04 (1×⅔H, dd, J=14, 6 Hz), 3.60 (1H, m), 3.70 (1H, m), 3.72 (3×⅓H, S), 3.73 (3×⅔H, S), 3.95 (1×⅓H, dd, J=9, 8 Hz), 4.00 (1×⅓H, m), 4.11 (1×⅔H, m), 4.36 (1H, dd, J=8.5, 3.5 Hz), 4.52 (1×⅔H, dd, J=9, 8 Hz), 5.09 (1×⅔H, d, J=12.5 Hz), 5.12 (1×⅓H, d, J=12.5 Hz), 5.13 (1×⅔H, d, J=12.5 Hz), 5.24 (1×⅓H, d, J=12.5 Hz), 6.87 (2×⅓H, d, J=8.5 Hz), 6.90 (2×⅔H, d, J=8.5 Hz), 7.16 (2×⅓H, d, J=8.5 Hz), 7.24 (2×⅔H, d, J=8.5 Hz), 7.30-7.44 (5H, m), 8.20 (2H, br), 8.73 (1×⅔H, d, J=9 Hz), 8.82 (1×⅓H, d, J=9 Hz);

MASS (ES+): m/e 496.

Preparation 224

Compound (224) was obtained in a manner similar to Preparation 24.

¹H-NMR (300 MHz, CDCl₃, δ): 0.64 (3×/6H, d, J=7 Hz), 0.68 (3×⅙H, t, J=7 Hz), 0.79 (3×⅚H, t, J=7 Hz), 0.84 (3×⅚H, d, J=7 Hz), 1.18-2.24 (13H, m), 3.00 (2H, m), 3.55 (1H, m), 3.75 (3H, s), 3.90 (1H, m), 4.08 (1H, m), 4.25 (2H, brt, J=7 Hz), 4.47 (1H, dd, J=8, 2 Hz), 4.56-4.71 (2H, m), 5.10 (1×⅚H, d, J=12.5 Hz), 5.14 (1×⅙H, d, J=12.5 Hz), 5.18 (1×⅚H, d, J=12.5 Hz), 5.21 (1×⅙H, d, J=12.5 Hz), 5.23 (1H, m), 6.45 (1H, brd, J=9 Hz), 6.67 (1H, d, J=8 Hz), 6.79 (2×⅙H, d, J=8.5 Hz), 6.81 (2×⅚H, d, J=8.5 Hz), 7.07 (2×⅙H, d, J=8.5 Hz), 7.11 (2×⅚H, d, J=8.5 Hz), 7.28-7.46 (7H, m), 7.54 (1H, m), 8.02 (2×/1H, d, J=7.5 Hz);

MASS (ES+): m/e 829.

Preparation 225

Compound (225) was obtained in a manner similar to Preparation 17.

¹H-NMR (300 MHz, CDCl₃, δ): 0.78 (3H, t, J=7 Hz), 0.84 (3H, d, J=6 Hz), 1.16-2.24 (13H, m), 2.90-3.10 (2H, m), 3.54 (1H, m), 3.74 (3H, s), 3.92-4.19 (2H, m), 4.28 (2H, m), 4.40-4.52 (2H, m), 4.65 (1H, m), 5.40 (1H, brd, J=7.5 Hz), 6.78 (2×1H, d, J=8.5 Hz), 6.86 (1H, brd, J=8 Hz), 6.94 (1H, brd, J=8 Hz), 7.11 (2×1H, brd, J=8.5 Hz), 7.42 (2×1H, dd, J=7.5, 7.5 Hz), 7.55 (1H, dd, J=7.5, 7.5 Hz), 8.02 (2×1H, d, J=7.5 Hz);

MASS (ES−): m/e 737.

Preparation 226

Compound (226) was obtained in a manner similar to Preparation 18.

¹H-NMR (300 MHz, CDCl₃, δ): 0.74 (3H, t, J=7 Hz), 0.88 (3H, d, J=6.5 Hz), 1.02 (1H, m), 1.20-1.46 (4H, m), 1.60-2.18 (8H, m), 2.91 (1H, dd, J=13.5, 8 Hz), 3.08 (1H, dd, J=13.5, 6.5 Hz), 3.48 (1H, m), 3.96 (1H, m), 4.14-4.35 (5H, m), 5.03 (1H, m), 6.67 (2×1H, d, J=8.5 Hz), 7.26 (2×1H, d, J=8.5 Hz), 7.40 (2×1H, dd, J=7.5, 7.5 Hz), 7.52 (1H, dd, J=7.5, 7.5 Hz), 8.02 (2×1H, d, J=7.5 Hz), 8.04 (2H, br), 8.20 (1H, br), 8.47 (1H, br);

MASS (ES−): m/e 637.

Preparation 227

Compound (227) was obtained in a manner similar to Preparation 76.

¹H-NMR (300 MHz, CDCl₃, δ): 0.84 (3H, d, J=7 Hz), 0.86 (3H, t, J=7 Hz), 1.09 (1H, m), 1.31-2.02 (10H, m), 2.24-2.46 (6H, m), 2.78 (1H, dd, J=14, 7.5 Hz), 3.15 (1H, dd, J=14, 7.5 Hz), 3.51 (1H, m), 3.76 (3H, s), 4.02 (1H, m), 4.22-4.34 (3H, m), 4.48 (1H, dd, J=10.5, 10.5 Hz), 4.64-4.76 (2H, m), 6.25 (1H, d, J=10 Hz), 6.28 (1H, d, J=10.5 Hz), 6.79 (2×1H, d, J=8.5 Hz), 7.11 (2×1H, d, J=8.5 Hz), 7.22 (1H, d, J=10 Hz), 7.44 (2×1H, dd, J=7.5, 7.5 Hz), 7.56 (1H, m), 8.02 (2×1H, dd, J=7.5, 1.5 Hz);

MASS (ES−): m/e 619.

Preparation 228

Compound (228) was obtained in a manner similar to Preparation 77.

¹H-NMR (300 MHz, CDCl₃, δ): 0.84 (3H, d, J=6.5 Hz), 0.86 (3H, t, J=7 Hz), 1.10 (1H, m), 1.22-2.02 (10H, m), 2.24-2.46 (2H, m), 2.79 (1H, dd, J=14.5, 7.5 Hz), 3.15 (1H, dd, J=14.5, 7.5 Hz), 3.51 (1H, m), 3.61 (2H, brt, J=6 Hz), 3.78 (3H, S), 4.02 (1H, m), 4.27 (1H, dt, J=10, 7.5 Hz), 4.48 (1H, dd, J=10.5, 10 Hz), 4.64-4.76 (2H, m), 6.31 (1H, d, J=10.5 Hz), 6.38 (1H, d, J=10 Hz), 6.81 (2×1H, d, J=8.5 Hz), 7.12 (2×1H, d, J=8.5 Hz), 7.22 (1H, d, J=10 Hz);

MASS (ES−): m/e 515.

Preparation 229

Compound (229) was obtained in a manner similar to Preparation 78.

¹H-NMR (300 MHz, CDCl₃, δ): 0.84 (3H, t, J=6.6 Hz), 0.86 (3H, t, J=7.3 Hz), 1.09 (1H, m), 1.20-2.02 (10H, m), 2.24-2.46 (2H, m), 2.79 (1H, dd, J=14.3, 7.9 Hz), 3.15 (1H, dd, J=14.3, 7.3 Hz), 3.51 (1H, m), 3.61 (2H, t, J=6.4 Hz), 3.78 (3H, s), 4.02 (1H, m), 4.27 (1H, dt, J=10.3, 7.6 Hz), 4.48 (1H, dd, J=11.0, 10.5 Hz), 4.69 (1H, ddd, J=9.9, 7.9, 7.3 Hz), 4.72 (1H, dd, J=8.0, 2.0 Hz), 6.31 (1H, d, J=10.5 Hz), 6.37 (1H, d, J=9.9 Hz), 6.81 (2×1H, d, J=8.4 Hz), 7.12 (2×1H, d, J=8.4 Hz), 7.22 (1H, d, J=10.3 Hz);

MASS (ES−): m/e 515.

Preparation 230

Compound (230) was obtained in a manner similar to Preparation 78. The obtained compound was used in Example 102.

¹H-NMR (300 MHz, CDCl₃, δ): 0.85 (3H, d, J=6.6 Hz), 0.87 (3H, t, J=7.3 Hz), 1.10 (1H, m), 1.44-2.06 (8H, m), 2.25-2.54 (4H, m), 2.80 (1H, dd, J=14.5, 8 Hz), 3.16 (1H, dd, J=14.5, 7.7 Hz), 3.52 (1H, m), 4.03 (1H, m), 4.28 (1H, dt, J=10, 7 Hz), 4.49 (1H, dd, J=10.7, 10.6 Hz), 4.69 (1H, ddd, J=9.8, 8, 7.7 Hz), 4.74 (1H, m), 6.28 (1H, d, J=10.6 Hz), 6.32 (1H, d, J=9.8 Hz), 6.81 (2×1H, d, J=8.7 Hz), 7.12 (2×1H, d, J=8.7 Hz), 7.24 (1H, d, J=10 Hz), 9.73 (1H, s);

MASS (ES−): m/e 513.

Preparation 231

Compound (231) was obtained in a manner similar to Preparation 24.

¹H-NMR (300 MHz, CDCl₃, δ): 1.27-1.97 (10H, m), 1.41 (9×⅙H, s), 1.43 (9×⅚H, s), 2.64 (1H, m), 2.70-3.08 (4H, m), 3.56 (1H, m), 3.71 (3×⅙H, s), 3.73 (3×⅚H, s), 4.06 (1H, m), 4.27 (2H, brt, J=7 Hz), 4.31 (1H, dd, J=8, 4 Hz), 4.68 (1H, m), 4.90 (1H, m), 5.10 (1H, d, J=12 Hz), 5.16 (1H, d, J=12 Hz), 5.18 (1H, d, J=7 Hz), 6.68 (2×⅙H, d, J=8.5 Hz), 6.73-6.92 (2H, m), 6.80 (2×⅚H, d, J=8.5 Hz), 7.08 (2H, d, J=8.5 Hz), 7.12-7.38 (9H, m), 7.42 (2H, dd, J=7.5, 7.5 Hz), 7.55 (1H, dd, J=7.5, 7.5 Hz), 8.03 (2H, d, J=7.5 Hz);

MASS (ES+): m/e 863.

Preparation 232

Compound (232) was obtained in a manner similar to Preparation 17.

¹H-NMR (300 MHz, CDCl₃, δ): 1.18-2.14 (10H, m), 1.41 (3×3H, s), 2.36 (3H, s), 2.68 (1H, m), 2.84-3.10 (4H, m), 3.72 (1H, m), 3.74 (3H, s), 4.06 (1H, m), 4.22-4.36 (3H, m), 4.70 (1H, m), 4.81 (1H, m), 5.29 (1H, brd, J=7.5 Hz), 6.78 (2×1H, d, J=8.5 Hz), 6.92 (1H, br), 7.04 (2×1H, brd, J=8.5 Hz), 7.14-7.32 (5H, m), 7.42 (2×1H, dd, J=7.5, 7.5 Hz), 7.48-7.60 (2H, m), 8.02 (2×1H, dd, J=7.5, 1.5 Hz);

MASS (ES−): m/e 771.

Preparation 233

Compound (233) was obtained in a manner similar to Preparation 18.

¹H-NMR (300 MHz, CDCl₃, δ): 1.12-1.98 (10H, m), 2.70-2.90 (2H, m), 2.91-3.12 (3H, m), 3.65 (3H, s), 4.07-4.34 (4H, m), 4.58 (1H, m), 5.07 (1H, m), 6.75 (2×1H, d, J=8.5 Hz), 7.13-7.30 (7H, m), 7.40 (2×1H, dd, J=7.5, 7.5 Hz), 7.52 (1H, dd, J=7.5, 7.5 Hz), 7.98-8.12 (2H, br), 8.02 (2×1H, d, J=7.5 Hz);

MASS (ES−): m/e 671.

Preparation 234

Compound (234) was obtained in a manner similar to Preparation 76.

¹H-NMR (300 MHz, CDCl₃, δ): 1.44 (2H, m), 1.66-1.96 (6H, m), 2.13-2.40 (2H, m), 2.77 (1H, dd, J=14, 7 Hz), 2.87 (1H, dd, J=13, 5 Hz), 3.02-3.24 (3H, m), 3.77 (3H, s), 3.94 (1H, m); 4.24-4.35 (2H, m), 4.61 (1H, dd, J=8, 2.5 Hz), 4.69 (1H, m), 5.06 (1H, ddd, J=10, 10, 5 Hz), 6.24 (1H, d, J=10 Hz), 6.44 (1H, d, J=10 Hz), 6.81 (2×1H, d, J=8.5 Hz), 7.09-7.32 (8H, m), 7.44 (2×1H, dd, J=7.5, 7.5 Hz), 7.56 (1H, m), 8.03 (2H, dd, J=7.5, 1.5 Hz);

MASS (ES−) m/e 653.

Preparation 235

Compound (235) was obtained in a manner similar to Preparation 77.

¹H-NMR (300 MHz, CDCl₃, δ): 1.24-1.91 (8H, m), 2.10-2.40 (2H, m), 2.78 (1H, dd, J=14, 7 Hz), 2.87 (1H, dd, J=13.5, 5.5 Hz), 3.02-3.24 (3H, m), 3.63 (2H, brt, J=6 Hz), 3.78 (3H, s), 3.94 (1H, m), 4.28 (1H, dt, J=10, 8 Hz), 4.61 (1H, dd, J=8, 3 Hz), 4.69 (1H, m), 5.06 (1H, ddd, J=10, 10, 5.5 Hz), 6.35 (1H, d, J=10 Hz), 6.46 (1H, d, J=10 Hz), 6.82 (2×1H, d, J=8.5 Hz), 7.09-7.32 (8H, m);

MASS (ES−): m/e 549.

Preparation 236

Compound (236) was obtained in a manner similar to Preparation 78. The obtained compound was used in Example 105.

¹H-NMR (300 MHz, CDCl₃, δ): 1.48-1.90 (4H, m), 2.10-2.50 (4H, m), 2.78 (1H, dd, J=14, 7 Hz), 2.87 (1H, dd, J=13.5, 5 Hz), 3.07 (1H, m), 3.16 (1H, dd, J=14, 8.5 Hz), 3.18 (1H, dd, J=13.5, 11 Hz), 3.78 (3H, s), 3.94 (1H, m), 4.28 (1H, dt, J=10.3, 7.3 Hz), 4.62 (1H, dd, J=8, 2.5 Hz), 4.68 (1H, ddd, J=10, 8.5, 7 Hz), 5.06 (1H, ddd, J=11, 10, 5 Hz), 6.32 (1H, d, J=10 Hz), 6.82 (2×1H, d, J=9 Hz), 7.09-7.32 (8H, m), 9.74 (1H, t, J=1 Hz);

MASS (ES−): m/e 547.

Preparation 237

Compound (237) was obtained in a manner similar to Preparation 14.

¹H-NMR (300 MHz, CDCl₃, δ): 1.40 (3×3H, s), 1.80 (1H, m), 1.90-2.11 (3H, m), 3.12 (1H, m), 3.73 (1H, m), 4.48 (1H, m), 5.17 (1H, d, J=12 Hz), 5.23 (1H, d, J=12 Hz), 5.43 (1H, d, J=7 Hz), 6.12 (1H, d, J=7 Hz), 7.23-7.45 (10H, m);

MASS (ES+): m/e 439.

Preparation 238

Compound (238) was obtained in a manner similar to Preparation 21.

¹H-NMR (300 MHz, CDCl₃, δ): 1.72-2.10 (4H, m), 2.71 (1H, m), 3.82 (1H, m), 4.46 (1H, m), 5.12 (1H, dd, J=12.5 Hz), 5.22 (1H, dd, J=12.5 Hz), 5.50 (1H, s), 7.30-7.54 (10H, m), 8.66 (2H, brs);

MASS (ES+): m/e 339.

Preparation 239

Compound (239) was obtained in a manner similar to Preparation 22.

¹H-NMR (300 MHz, CDCl₃, δ): 0.71 (3H, t, J=7.5 Hz), 1.36 (3×3H, brs), 1.42 (3H, s), 1.56-2.10 (6H, m), 3.11 (1H, m), 3.74 (1H, m), 4.49 (1H, m), 5.16 (2H, s), 5.64 (1H, d, J=6.5 Hz), 7.21-7.43 (11H, m), 7.63 (1H, d, J=6.5 Hz);

MASS (ES+): m/e 538.

Preparation 240

Compound (240) was obtained in a manner similar to Preparation 23.

¹H-NMR (300 MHz, CDCl₃, δ): 0.95 (3H, t, J=7 Hz), 1.60 (3H, s), 1.70-2.19 (6H, m), 3.09 (1H, m), 3.78 (1H, m), 4.48 (1H, m), 5.16 (2H, s), 5.73 (1H, d, J=6.5 Hz), 7.22-7.45 (10H, m), 7.62 (1H, d, J=6.5 Hz), 8.02 (2H, brs);

MASS (ES+): m/e 438.

Preparation 241

Compound (241) was obtained in a manner similar to Preparation 24.

¹H-NMR (300 MHz, CDCl₃, δ): 0.72 (3H, t, J=7.5 Hz), 1.36-2.42 (12H, m), 1.41 (3×3H, s), 1.47 (3H, s), 3.11 (1H, m), 3.73 (1H, m), 4.04 (1H, m), 4.28 (2H, t, J=6 Hz), 4.50 (1H, m), 5.07 (1H, br), 5.16 (1H, d, J=12.5 Hz), 5.19 (1H, d, J=12.5 Hz), 5.62 (1H, d, J=6 Hz), 7.03 (1H, s), 7.26-7.48 (13H, m), 7.54 (1H, m), 8.01 (2×1H, dd, J=7, 1.5 Hz);

MASS (ES+): m/e 793 (M+Na).

Preparation 242

Compound (242) was obtained in a manner similar to Preparation 17.

¹H-NMR (300 MHz, CDCl₃, δ): 0.68 (3H, brt, J=7 Hz), 1.34-2.21 (12H, m), 1.42 (3×3H, s), 1.44 (3H, s), 3.12 (1H, m), 3.77 (1H, m), 4.05 (1H, m), 4.33 (2H, brt, J=6 Hz), 4.46 (1H, m), 5.14 (1H, br), 5.67 (1H, d, J=7 Hz), 6.89 (1H, brs), 7.24-7.47 (7H, m), 7.56 (1H, m), 7.69 (1H, brd, J=7 Hz), 8.03 (2×1H, dd, J=7.5, 1 Hz);

MASS (ES−): m/e 679.

¹H-NMR (300 MHz, DMSO-d₆, δ): 0.57 (3× 7/9H, t, J=7.5 Hz), 0.62 (3× 2/9H, t, J=7.5 Hz), 1.26-2.08 (12H, m), 1.33 (3H, s), 1.34 (3×3H, s), 3.12 (1H, m), 3.75 (1H, m), 3.88 (1H, m), 4.19-4.32 (3H, m), 5.58 (1× 2/9H, d, J=7.5 Hz), 5.68 (1× 7/9H, d, J=7.5 Hz), 6.94 (1H, d, J=8.5 Hz), 7.22-7.41 (5H, m), 7.52 (2×1H, dd, J=7.5, 7.5 Hz), 7.66 (1H, m), 7.78 (1H, s), 7.96 (2×1H, dd, J=7.5, 1.5 Hz).

Preparation 243

Compound (243) was obtained in a manner similar to Preparation 18.

¹H-NMR (300 MHz, CDCl₃, δ): 0.70 (3H, t, J=7 Hz), 1.42 (3H, s), 1.54-2.16 (12H, m), 3.09 (1H, m), 3.83 (1H, m), 4.26-4.54 (4H, m), 5.77 (1H, d, J=7 Hz), 7.25-7.42 (7H, m), 7.51 (1H, dd, J=7.5, 7.5 Hz), 7.58 (1H, br), 7.91 (2H, brs), 8.02 (2×1H, d, J=7.5 Hz), 8.62 (1H, s);

MASS (ES+): m/e 581.

¹H-NMR (300 MHz, DMSO-d₆, δ): 0.59 (3H, t, J=7.5 Hz), 1.32-1.92 (12H, m), 1.37 (3H, s), 3.07 (1H, m), 3.74 (1H, m), 3.88 (1H, m), 1.25 (1H, dd, J=8, 2 Hz), 4.30 (2H, t, J=6 Hz), 5.65 (1H, d, J=7 Hz), 7.25-7.40 (5H, m), 7.52 (1H, dd, J=7.5, 7.5 Hz), 7.66 (1H, m), 7.90 (2H, d, J=7 Hz), 7.98 (2×1H, dd, J=7.5, 1.5 Hz), 8.15 (2H, br), 8.40 (1H, s).

Preparation 244

Compound (244) was obtained in a manner similar to Preparation 76.

¹H-NMR (300 MHz, CDCl₃, δ): 0.90 (3H, t, J=7.3 Hz), 1.3-6 (3H, s), 1.48 (2H, m), 1.58-2.56 (10H, m), 3.76 (1H, m), 4.04 (1H, m), 4.30 (1H, m), 4.32 (2H, t, J=6.5 Hz), 4.76 (1H, m), 5.99 (1H, s), 6.20 (1H, d, J=10 Hz), 7.17 (1H, d, J=10 Hz), 7.28-7.49 (7H, m), 7.56 (1H, m), 8.04 (2H, m), 8.10 (1H, d, J=10 Hz);

MASS (ES+): m/e 563.

Preparation 245

Compound (245) was obtained in a manner similar to Preparation 77.

¹H-NMR (300 MHz, CDCl₃, δ): 0.92 (3H, t, J=7.5 Hz), 1.36 (3H, s), 1.39 (2H, m), 1.52-1.71 (4H, m), 1.79-2.06 (3H, m), 2.10-2.53 (4H, m), 3.65 (1H, dt, J=6, 6 Hz), 3.74 (1H, m), 4.04 (1H, m), 4.27 (1H, dt, J=10, 7.5 Hz), 4.75 (1H, dd, J=8, 2 Hz), 5.97 (1H, s), 6.19 (1H, d, J=10.5 Hz), 7.14 (1H, d, J=10 Hz), 7.28-7.43 (5H, m), 8.08 (1H, d, J=10.5 Hz);

MASS (ES+): m/e 459.

Preparation 246

Compound (246) was obtained in a manner similar to Preparation 78. The obtained compound was used in Example 108.

¹H-NMR (300 MHz, CDCl₃, δ): 0.92 (3H, t, J=7.4 Hz), 1.26 (3H, s), 1.52-1.74 (3H, m), 1.78-2.06 (3H, m), 2.12-2.54 (6H, m), 3.74 (1H, dt, J=10, 7 Hz), 4.04 (1H, m), 4.28 (1H, dt, J=10.5, 7 Hz), 4.76 (1H, dd, J=8, 2 Hz), 6.05 (1H, s), 6.18 (1H, d, J=10 Hz), 7.18 (1H, d, J=10 Hz), 7.28-7.42 (5H, m), 8.02 (1H, d, J=10 Hz), 9.77 (1H, brs);

MASS (ES−): m/e 455.

Preparation 247

Compound (247) was obtained in a manner similar to Preparation 20.

¹H-NMR (300 MHz, CDCl₃, δ): 0.60-2.30 (17H, m), 1.41 (9×¼H, s), 1.44 (9×¾H, s), 3.42-3.64 (1H, m), 3.84 (1H, m), 4.27 (1×¼H, m), 4.47 (1×¾H, m), 4.58 (1H, m), 4.97 (1H, m), 5.13 (1H, d, J=12.5 Hz), 5.13-5.23 (1H, m), 5.19 (1H, d, J=12.5 Hz), 7.28-7.42 (5H, m);

MASS (ES+): m/e 459.

Preparation 248

Compound (248) was obtained in a manner similar to Preparation 21.

¹H-NMR (300 MHz, DMSO-d₆, δ): 0.68-1.34 (5H, m), 1.38-1.76 (7H, m), 1.82-2.06 (4H, m), 2.18 (1H, m), 3.42 (1H, m), 3.80 (1H, m), 4.25 (1H, brt, J=6 Hz), 4.39 (1H, dd, J=8.5, 2.5 Hz), 5.10 (1H, d, J=12.5 Hz), 5.18 (1H, d, J=12.5 Hz), 7.13-7.44 (5H, m), 8.20 (2H, brs);

MASS (ES+): m/e 359.

Preparation 249

Compound (249) was obtained in a manner similar to Preparation 22.

¹H-NMR (300 MHz, DMSO-d₆, δ): 0.68 (3×⅔H, brt, J=7 Hz), 0.77-2.30, (19H, m), 0.84 (3×⅓H, brt, J=7 Hz), 1.24 (3×⅓H, s), 1.27 (3×⅔H, s), 1.33 (9×⅓H, s), 1.36 (9×⅔H, s), 3.50 (1H, m), 3.69 (1H, m), 4.31 (1H, dd, J=8, 3 Hz), 4.42 (1×⅓H, m), 4.69 (1×⅔H, m), 5.03 (1H, d, J=12.5 Hz), 5.10 (1H, d, J=12.5 Hz), 6.54 (1×⅓H, br), 6.67 (1×⅔H, br), 7.31-7.42 (5H, m), 7.44 (1×⅓H, d, J=8 Hz), 7.70 (1×⅔H, d, J=8 Hz);

MASS (ES+): m/e 558.

Preparation 250

Compound (250) was obtained in a manner similar to Preparation 23.

¹H-NMR (300 MHz, DMSO-d₆, δ): 0.74 (3×¼H, t, J=7.5 Hz), 0.78 (3×¾H, t, J=7.5 Hz), 0.82-2.28 (19H, m), 1.44 (3×¼H, s), 1.47 (3×¾H, s), 3.56 (1H, m), 3.77 (1H, m), 4.33 (1H, dd, J=8.5, 3 Hz), 4.78 (1×¾H, m), 5.01 (1H, d, J=12.5 Hz), 5.04 (1×¼H, m), 5.16 (1H, d, J=12.5 Hz), 7.29-7.42 (5H, m), 8.15 (2H, brs), 8.46 (1×¾H, d, J=8.5 Hz), 8.62 (1×¼H, d, J=8.5 Hz);

MASS (ES+): m/e 458.

Preparation 251

Compound (251) was obtained in a manner similar to Preparation 24.

¹H-NMR (300 MHz, DMSO-d₆, δ): 0.57 (3H, t, J=7.3 Hz), 0.70-2.30 (25H, m), 1.34 (3H, s), 1.36 (3×3H, s), 3.52 (1H, m), 3.66-3.84 (2H, m), 4.24 (2H, t, J=6.5 Hz), 4.31 (1H, dd, J=9, 3 Hz), 4.76 (1H, m), 5.01 (1H, d, J=12.5 Hz), 5.12 (1H, d, J=12.5 Hz), 7.14 (1H, m), 7.29-7.42 (5H, m), 7.51 (2H, m), 7.65 (1H, m), 7.70 (1H, s), 7.80 (1H, d, J=6.5 Hz), 7.95 (2×1H, d, J=7 Hz);

MASS (ES+): m/e 791.

Preparation 252

Compound (252) was obtained in a manner similar to Preparation 17.

¹H-NMR (300 MHz, CDCl₃, δ): 0.76-2.36 (25H, m), 0.80 (3H, t, J=7.5 Hz), 1.43 (3×3H, s), 1.48 (3H, s), 3.50 (1H, m), 3.93 (1H, m), 4.02 (1H, m), 4.33 (2H, t, J=6.5 Hz), 4.59 (1H, m), 4.86 (1H, m), 5.23 (1H, m), 6.91 (1H, s), 7.16 (1H, d, J=8.5 Hz), 7.43 (2×1H, dd, J=8, 8 Hz), 7.56 (1H, m), 8.03 (2×1H, dd, J=8, 1.5 Hz);

MASS (ES−): m/e 699.

Preparation 253

Compound (253) was obtained in a manner similar to Preparation 18.

¹H-NMR (300 MHz, DMSO-d₆, δ): 0.67 (3×½H, t, J=7.5 Hz), 0.68 (3×½H, t, J=7.5 Hz), 0.72-2.32 (25H, m), 1.40 (3×½H, s), 1.41 (3×½H, S), 3.33 (1H, m), 3.48 (1×½H, m), 3.71 (1×½H, m), 3.96 (1H, m), 4.18 (1×½H, dd, J=8.5, 2.5 Hz), 4.27 (2×½H, t, J=6.2 Hz), 4.29 (2×½H, t, J=6.2 Hz), 4.42 (11×/2H, m), 4.75 (1×½H, m), 4.81 (1×½H, d, J=8, 2 Hz), 7.53 (2×½H, dd, J=7.5, 7.5 Hz), 7.67 (1H, dd, J=7.5, 7.5 Hz), 7.75 (1×½H, d, J=8.5 Hz), 7.88 (1×½H, d, J=8.5 Hz), 7.96 (2×1H, d, J=7.5 Hz), 8.05 (2H, br), 8.14 (1×½H, s), 8.16 (1×½H, s);

MASS (ES+): m/e 601.

Preparation 254

Compound (254) was obtained in a manner similar to Preparation 76.

¹H-NMR (300 MHz, CDCl₃, δ): 0.85 (3H, t, J=7 Hz), 0.96 (2H, m), 1.08-1.26 (4H, m), 1.28 (3H, s), 1.45 (2H, m), 1.55-1.98 (13H, m), 2.07-2.42 (4H, m), 3.52 (1H, m), 3.96 (1H, m), 4.24 (1H, ddd, J=10, 8, 8 Hz), 4.31 (2H, t, J=6 Hz), 4.74 (1H, m), 5.00 (1H, ddd, J=10, 8, 8 Hz), 5.83 (1H, s), 7.14 (1H, d, J=10 Hz), 7.34 (1H, d, J=10 Hz), 7.43 (2×1H, dd, J=7.5, 7.5 Hz), 7.56 (1H, dd, J=7.5, 7.5 Hz), 8.03 (2×1H, d, J=7.5 Hz);

MASS (ES−): m/e 581.

Preparation 255

Compound (255) was obtained in a manner similar to Preparation 77.

¹H-NMR (300 MHz, CDCl₃, δ): 0.87 (3H, t, J=7.3 Hz), 0.96 (2H, m), 1.08-1.51 (6H, m), 1.53-2.00 (11H, m), 2.09-2.43 (4H, m), 3.51 (1H, ddd, J=10, 7.5, 7 Hz), 3.65 (2H, brt, J=5 Hz), 3.96 (1H, m), 4.23 (1H, ddd, J=10, 8, 7 Hz), 4.74 (1H, dd, J=8, 2 Hz), 4.99 (1H, ddd, J=10, 8, 8 Hz), 6.01 (1H, s), 7.16 (1H, d, J=10 Hz), 7.35 (1H, d, J=10 Hz);

MASS (ES−): m/e 477.

Preparation 256

Compound (256) was obtained in a manner similar to Preparation 78. The obtained compound was used in Examples 111, 114.

¹H-NMR (300 MHz, CDCl₃, δ): 0.87 (3H, t, J=7.3 Hz), 0.96 (2H, m), 1.08-1.35 (4H, m), 1.30 (3H, s), 1.50-2.02 (13H, m), 2.10-2.44 (4H, m), 2.49 (2H, m), 3.52 (1H, dt, J=10, 7.3 Hz), 3.96 (1H, m), 4.23 (1H, ddd, J=10, 7.5, 7 Hz), 4.74 (1H, dd, J=8, 2 Hz), 4.99 (1H, dt, J=10, 7.5 Hz), 5.89 (1H, s), 7.16 (1H, d, J=10 Hz), 7.29 (1H, d, J=10 Hz), 9.76 (1H, t, J=1 Hz);

MASS (ES−): m/e 475.

Preparation 257

Compound (257) was obtained in a manner similar to Preparation 22.

¹H-NMR (300 MHz, CDCl₃, δ): 1.18-2.18 (14H, m), 1.41 (9×¾H, s), 1.48 (9×¼H, s), 2.64 (1H, m), 2.88 (1H, m), 3.03 (1×¾H, m), 3.15 (1×¼H, m), 3.50 (1×¾H, m), 3.58 (1×¼H, m), 4.17 (1H, dd, J=8, 3.5 Hz), 4.68-5.14 (3H, m), 6.86-7.44 (12H, m);

MASS (ES+): m/e 578.

Preparation 258

Compound (258) was obtained in a manner similar to Preparation 23.

¹H-NMR (300 MHz, DMSO-d₆, δ): 0.82-2.14 (14H, m), 1.35 (9×⅚H, s), 1.45 (9×⅚H, s), 2.83 (1H, dd, J=13, 5 Hz), 2.92 (1H, dd, J=13, 6.5 Hz), 3.17 (1H, m), 3.40 (1×⅙H, m), 3.53 (1×⅚H, m), 4.06 (1×⅚H, dd, J=8.5, 3.5 Hz), 4.47 (1×⅙H, m), 4.73 (1×⅚H, m), 4.84 (1×⅙H, m), 7.11-7.30 (5H, m), 8.30 (1H, d, J=8.5 Hz);

MASS (ES+): m/e 444.

Preparation 259

Compound (259) was obtained in a manner similar to Preparation 24.

¹H-NMR (300 MHz, CDCl₃, δ): 1.12-2.28 (20H, m), 1.42 (3×3H, s), 1.44 (3×3H, s), 2.69 (1H, m), 2.92 (1H, dd, J=13.5, 9.5 Hz), 3.03 (1H, dd, J=13.5, 5 Hz), 3.51 (1H, m), 3.93-4.20 (2H, m), 4.33 (2H, brt, J=6 Hz), 4.88 (1H, m), 5.17 (1H, br), 6.51 (1H, brs), 7.12-7.32 (6H, m), 7.44 (2×1H, dd, J=7.5, 7.5 Hz), 7.56 (1H, dd, J=7.5, 7.5 Hz), 8.03 (2×1H, d, J=7.5 Hz);

MASS (ES−): m/e 775.

Preparation 260

Compound (260) was obtained in a manner similar to Preparation 57.

¹H-NMR (300 MHz, CDCl₃, δ): 1.06-2.10 (19H, m), 2.32 (1H, m), 2.87-3.07 (3H, m), 3.74 (1H, m), 4.0.8-4.42 (4H, m), 4.74 (1H, m), 7.14-7.32 (6H, m), 7.38-7.62 (4H, m), 7.77 (2H, br), 8.02 (2×1H, d, J=8 Hz);

MASS (ES+): m/e 620.

Preparation 261

Compound (261) was obtained in a manner similar to Preparation 76.

¹H-NMR (300 MHz, CDCl₃, δ): 1.26-1.96 (16H, m), 2.04 (1H, m), 2.17 (1H, m), 2.30 (1H, m), 2.62 (1H, m), 2.95 (1H, dd, J=13.6 Hz), 3.21 (1H, m), 3.25 (1H, dd, J=13, 10 Hz), 3.92 (1H, m), 4.25 (1H, ddd, J=10, 8, 7.5 Hz), 4.32 (2H, t, J=6.5 Hz), 4.66 (1H, m), 5.16 (1H, ddd, J=10, 10, 6 Hz), 5.70 (1H, s), 7.15-7.32 (6H, m), 7.38 (1H, d, J=10 Hz), 7.44 (2H, m), 7.56 (1H, m), 8.03 (2H, m);

MASS (ES−): m/e 601.

Preparation 262

Compound (262) was obtained in a manner similar to Preparation 77.

¹H-NMR (300 MHz, CDCl₃, δ): 1.22-1.93 (16H, m), 2.04 (1H, m), 2.16 (1H, m), 2.30 (1H, m), 2.63 (1H, m), 2.95 (1H, dd, J=13.5, 6 Hz), 3.20 (1H, m), 3.26 (1H, dd, J=13.5, 10 Hz), 3.66 (2H, t, J=6.5 Hz), 3.92 (1H, m), 4.24 (1H, ddd, J=10, 8, 8 Hz), 4.64 (1H, m), 5.16 (1H, ddd, J=10, 10, 6 Hz), 5.84 (1H, s), 7.15-7.32 (6H, m), 7.38 (1H, d, J=10 Hz);

MASS (ES−): m/e 497.

Preparation 263

Compound (263) was obtained in a manner similar to Preparation 78. The obtained compound was used in Examples 117, 120.

¹H-NMR (300 MHz, CDCl₃, δ): 1.20-1.93 (14H, m), 1.98-2.67 (6H, m), 2.95 (1H, dd, J=14, 5 Hz), 3.20 (1H, m), 3.24 (1H, dd, J=14, 10 Hz), 3.92 (1H, m), 4.24 (1H, m), 4.66 (1H, m), 5.16 (1H, ddd, J=10, 5, 5 Hz), 5.76 (1H, s), 7.15-7.40 (7H, m), 9.77 (1H, t, J=1 Hz);

MASS (ES−): m/e 495.

Preparation 264

Compound (264) was obtained in a manner similar to Preparation 77. The obtained compound was used in Examples 117, 120.

¹H-NMR (300 MHz, CDCl₃, δ): 1.24-1.90 (14H, m), 1.96-2.25 (2H, m), 2.32 (1H, m), 2.50 (2H, m), 2.60 (1H, m), 2.95 (1H, dd, J=13.5, 6 Hz), 3.20 (1H, m), 3.24 (1H, dd, J=13.5, 10 Hz), 3.93 (1H, m), 4.24 (1H, m), 4.66 (1H, dd, J=8, 2.5 Hz), 5.16 (1H, ddd, J=10, 10, 6 Hz), 5.76 (1H, s), 7.16-7.34 (6H, m), 7.34 (1H, d, J=10 Hz), 9.77 (1H, t, J=1 Hz);

MASS (ES−): m/e 495.

Preparation 265

Compound (265) was obtained in a manner similar to Preparation 21.

¹H-NMR (300 MHz, CDCl₃, δ): 1.52 (1H, m), 1.66-2.01 (3H, m), 2.71 (1H, m), 2.96 (1H, dd, J=13.5, 8 Hz), 3.14 (1H, dd, J=13.5, 6 Hz), 3.55 (1H, m), 4.26 (1H, dd, J=8.5, 3.5 Hz), 4.41 (1H, br), 5.08 (1H, d, J=12.5 Hz), 5.19 (1H, d, J=12.5 Hz), 7.16-7.46 (10H, m), 8.41 (2H, brs);

MASS (ES+): m/e 353.

Preparation 266

Compound (266) was obtained in a manner similar to Preparation 22.

¹H-NMR (300 MHz, CDCl₃, δ): 0.75-2.00 (17H, m), 1.41 (9×¼H, s), 1.46 (9×¾H, s), 2.63 (1H, m), 2.93 (1H, dd, J=13.5, 9.5 Hz), 3.06 (1H, dd, J=13.5, 6 Hz), 3.50 (1×¾H, m), 3.60 (1×¼H, m), 4.04 (1×¼H, m), 4.19 (1×¾H, m), 4.36 (1H, dd, J=8, 4 Hz), 4.75 (1H br), 4.94 (1H, ddd, J=9.5, 7, 6 Hz), 5.10 (1H, d, J=12.5 Hz), 5.19 (1H, d, J=12.5 Hz), 6.82 (1×¾H, brd, J=7 Hz), 7.04 (1×¼H, brd, J=7 Hz), 7.14-7.41 (10H, m);

MASS (ES−): m/e 604.

Preparation 267

Compound (267) was obtained in a manner similar to Preparation 23.

¹H-NMR (300 MHz, CDCl₃, δ): 0.68-2.32 (17H, m), 2.80 (½H, m), 2.95-3.16 (2H, m), 3.50-3.80 (1+½H, m), 4.26-4.46 (1×½H, m), 4.62 (1×½H, m), 4.86 (1×½H, m), 5.10-5.24 (2H, m), 5.36 (½H, m), 7.12-7.40 (10H, m), 8.16 (1H, br), 8.36-8.54 (1×½H, m), 8.75 (1×½H, br);

MASS (ES+): m/e 506.

Preparation 268

Compound (268) was obtained in a manner similar to Preparation 24.

¹H-NMR (300 MHz, CDCl₃, δ): 0.69-2.06 (23H, m), 1.42 (9× 1/7H, s), 1.43 (9× 6/7H, s), 2.72 (1H, m), 2.92-3.08 (2H, m), 3.57 (1H, m), 4.12 (1H, m), 4.25-4.40 (3H, m), 4.52 (1H, m), 4.93 (1H, m), 5.10 (1H, d, J=12.5 Hz), 5.17 (1H, d, J=12.5 Hz), 5.20 (1H, br), 6.39 (1× 1/7H, d, J=8.5 Hz), 6.58 (1× 6/7H, d, J=8.5 Hz), 6.86 (1H, brd, J=7 Hz), 7.15-7.39 (10H, m), 7.43 (2×1H, dd, J=7.5, 7.5 Hz), 7.55 (1H, m), 8.03 (2×1H, dd, J=7.5, 1.5 Hz);

MASS (ES−): m/e 837.

Preparation 269

Compound (269) was obtained in a manner similar to Preparation 17.

¹H-NMR (300 MHz, CDCl₃, δ): 0.64-2.12 (23H, m), 1.45 (3×3H, s), 2.67 (1H, m), 2.95-3.11 (2H, m), 3.71 (1H, m), 4.08 (1H, m), 4.26-4.64 (4H, m), 4.74 (1H, m), 5.89 (1H, br), 6.95 (1H, br), 7.13-7.34 (5H, m), 7.43 (2×1H, dd, J=7.5, 7.5 Hz), 7.56 (1H, dd, J=7.5, 7.5 Hz), 7.73 (1H, br), 8.04 (2×1H, d, J=7.5 Hz);

MASS (ES−): m/e 747.

Preparation 270

Compound (270) was obtained in a manner similar to Preparation 57.

¹H-NMR (300 MHz, CDCl₃, δ): 0.70-0.90 (2H, m), 1.94-1.30 (6H, m), 1.36-1.67 (7H, m), 1.70-2.18 (8H, m), 2.87-3.01 (2H, m), 3.11 (1H, m), 3.72 (1H, m), 3.96 (1H, m), 4.10 (1H, m), 4.33 (2H, t, J=6 Hz), 4.48-4.62 (2H, m), 7.18-7.34 (5H, m), 7.44 (2×1H, dd, J=7.5, 7.5 Hz), 7.55 (1H, dd, J=7.5, 7.5 Hz), 7.90 (1H, d, J=8 Hz), 8.04 (2×1H, d, J=7.5 Hz), 8.34 (2H, br), 9.07 (1H, d, J=7 Hz);

MASS (ES+): m/e 649.

Preparation 271.

Compound (271) was obtained in a manner similar to Preparation 76.

¹H-NMR (300 MHz, CDCl₃, δ): 0.91 (2H, m), 1.06-1.34 (5H, m), 1.36-1.99 (14H, m), 2.18 (1H, m), 2.31 (1H, m), 2.94 (1H, dd, J=13, 5 Hz), 3.10 (1H, m), 3.22 (1H, dd, J=13, 10 Hz), 3.93 (1H, m), 4.31 (1H, t, J=6.5 Hz), 4.31 (1H, m), 4.52 (1H, dt, J=10, 7.5 Hz), 4.62 (1H, m), 5.09 (1H, ddd, J=10, 10, 5 Hz), 6.06 (1H, d, J=10 Hz), 6.49 (1H, d, J=10 Hz), 7.15-7.32 (6H, m), 7.40-7.47 (2H, m), 7.52-7.59 (1H, m), 8.00-8.06 (2H, m);

MASS (ES−) m/e 629.

Preparation 272

Compound (272) was obtained in a manner similar to Preparation 77.

¹H-NMR (300 MHz, CDCl₃, δ): 0.92 (2H, m), 1.08-1.92 (19H, m), 2.18 (1H, m), 2.31 (1H, m), 2.94 (1H, dd, J=13.5, 5.5 Hz), 3.10 (1H, m), 3.22 (1H, dd, J=13.5, 10 Hz), 3.66 (1H, dt, J=6, 5 Hz), 3.94 (1H, m), 4.29 (1H, dt, J=10, 7 Hz), 4.52 (1H, dt, J=10, 7.5 Hz), 4.63 (1H, m), 5.09 (1H, ddd, J=10, 10, 5.5 Hz), 6.15 (1H, d, J=10 Hz), 6.51 (1H, d, J=10 Hz), 7.14-7.33 (6H, m);

MASS (ES−): m/e 525.

Preparation 273

Compound (273) was obtained in a manner similar to Preparation 78. The obtained compound was used in Examples 123, 126, 129.

¹H-NMR (300 MHz, CDCl₃, δ): 0.90 (2H, m), 1.10-1.32 (4H, m), 1.37-1.95 (13H, m), 2.11-2.55 (4H, m), 2.94 (1H, dd, J=13, 5 Hz), 3.09 (1H, m), 3.21 (1H, dd, J=13, 10 Hz), 3.94 (1H, m), 4.31 (1H, m), 4.52 (1H, dt, J=10, 7 Hz), 4.63 (1H, m), 5.08 (1H, ddd, J=10, 10, 5 Hz), 6.13 (0.6H, d, J=10 Hz), 6.32 (0.4H, d, J=10 Hz), 6.50 (0.6H, d, J=10 Hz), 6.61 (0.4H, d, J=10 Hz), 7.17-7.34 (6H, m), 9.76 (1H, t);

MASS (ES+): m/e 525.

Preparation 274

Compound (274) was obtained in a manner similar to Preparation 14.

¹H-NMR (300 MHz, CDCl₃, δ): 1.44 (9×⅕H, s), 1.46 (9×⅘H, s), 1.78-2.24 (6H, m), 2.69 (2H, t, J=8 Hz), 3.31 (1×⅘H, m), 3.60 (1×⅕H, m), 3.70 (1×⅘H, m), 4.25 (1×⅕H, m), 4.42 (1×⅘H, dd, J=8, 3 Hz), 4.54 (1×⅘H, m), 4.70 (1×⅕H, m), 4.93 (1×⅕H, m), 5.00 (1×⅕H, d, J=12.5 Hz), 5.07 (1×⅕H, d, J=12.5 Hz), 5.12 (1×⅘H, d, J=12.5 Hz), 5.20 (1×⅘H, d, J=12.5 Hz), 5.40 (1H, brd, J=8 Hz), 7.10-7.41 (10H, m);

MASS (ES+): m/e 467.

Preparation 275

Compound (275) was obtained in a manner similar to Preparation 21.

¹H-NMR (300 MHz, DMSO-d₆, δ): 1.80-2.10 (6H, m), 2.70 (2H, m), 3.40 (1H, m), 3.65 (1H, m), 4.25 (1H, m), 4.35 (1H, m), 5.10 (1H, d, J=12 Hz), 5.19 (1H, d, J=12 Hz), 7.05-7.44 (10H, m), 8.42 (2H, brs);

MASS (ES+): m/e 367.

Preparation 276

Compound (276) was obtained in a manner similar to Preparation 22.

¹H-NMR (300 MHz, DMSO-d₆, δ): 0.71 (3H, t, J=7.3 Hz), 1.28 (3×¼H, s), 1.29 (3×¾H, s), 1.34 (9×¼H, s), 1.36 (9×¾H, s), 1.70-2.62 (10H, m), 3.24-3.44 (3H, m), 3.58 (1H, m), 4.30 (1H, dd, J=9, 3.5 Hz), 4.60 (1H, m), 5.04 (1H, d, J=13 Hz), 5.10 (1H, d, J=13 Hz), 6.63 (1×¼H, brs), 6.80 (1×¾H, brs), 7.05-7.41 (10H, m), 7.58 (1×¾H, d, J=9 Hz), 7.92 (1×¼H, d, J=9 Hz);

MASS (ES+): m/e 566.

Preparation 277

Compound (277) was obtained in a manner similar to Preparation 23.

¹H-NMR (300 MHz, DMSO-d₆, δ): 0.80 (3H, t, J=7 Hz), 1.52 (3×⅕H, s), 1.54 (3×⅘H, s), 1.66-2.75 (8H, m), 3.39 (1H, m), 3.60 (1H, m), 4.33 (1H, dd, J=9, 3 Hz), 4.63 (1H, m), 5.00 (1×⅘H, d, J=13 Hz), 5.06 (1×⅕H, dd, J=13 Hz), 5.12 (1×⅕H, d, J=13 Hz), 5.16 (1×⅘H, d, J=13 Hz), 7.08 (1H, brd, J=7 Hz), 7.16-7.42 (9H, m), 8.16 (2×⅘H, brs), 8.20 (2×⅕H, brs), 8.57 (1×⅘H, d, J=8.5 Hz), 8.74 (1×⅕H, d, J=8.5 Hz);

MASS (ES+): m/e 466.

Preparation 278.

Compound (278) was obtained in a manner similar to Preparation 24.

¹H-NMR (300 MHz, CDCl₃, δ): 0.76 (3H, t, J=7 Hz), 1.43 (3×3H, s), 1.45-2.58 (14H, m), 1.53 (3H, s), 2.65 (2H, t, J=8 Hz), 3.32 (1H, m), 3.68 (1H, m), 4.08 (1H, m), 4.31 (2H, t, J=6 Hz), 4.44 (1H, dd, J=8, 2.5 Hz), 4.82 (1H, m), 5.12 (1H, m), 5.13 (2H, s), 6.78 (1H, brd, J=8 Hz), 7.01 (1H, s), 7.09-7.38 (10H, m), 7.39-7.47 (2H, m), 7.55 (1H, m), 8.00-8.06 (2H, m);

MASS (ES+): m/e 799.

Preparation 279

Compound (279) was obtained in a manner similar to Preparation 17.

¹H-NMR (300 MHz, CDCl₃, δ): 0.86 (3H, t, J=7.5 Hz), 1.42 (3×3H, s), 1.44-2.30 (14H, m), 1.46 (3H, s), 2.66 (2H, t, J=7 Hz), 3.26 (1H, m), 3.74 (1H, m), 4.02 (1H, m), 4.32 (2H, brt, J=6 Hz), 4.42 (1H, m), 4.77 (1H, m), 6.89 (1H, s), 7.11-7.31 (7H, m), 7.43 (2×1H, dd, J=7.5, 7.5 Hz), 7.55 (1H, m), 8.03 (2×1H, dd, J=7.5, 1.5 Hz);

MASS (ES−): m/e 707.

Preparation 280

Compound (280) was obtained in a manner similar to Preparation 18.

¹H-NMR (300 MHz, CDCl₃, δ): 0.86 (3H, t, J=7.5 Hz), 1.37 (3H, s), 1.58 (2H, m), 1.72-2.24 (12H, m), 2.60 (1H, m), 2.72 (1H, m), 3.19 (1H, m), 3.63 (1H, m), 4.09 (1H, m), 4.23-4.38 (3H, m), 4.61 (1H, m), 7.12-7.32 (6H, m), 7.42 (2×1H, dd, J=7.5, 7.5 Hz), 7.56 (1H, m), 7.60 (1H, brd, J=9 Hz), 7.78 (2H, br), 8.01 (2×1H, d, J=7.5 Hz);

MASS (ES+): m/e 609.

Preparation 281

Compound (281) was obtained in a manner similar to Preparation 76.

¹H-NMR (300 MHz, CDCl₃, δ): 0.85 (3H, t, J=7 Hz), 1.28 (3H, s), 1.45 (2H, m), 1.61-1.97 (6H, m), 1.98-2.43 (6H, m), 2.64 (2H, m), 3.32 (1H, m), 3.75 (1H, m), 4.24 (1H, dt, J=10, 7.5 Hz), 4.31 (1H, t, J=6.5 Hz), 4.72 (1H, m), 4.84 (1H, dt, J=10, 7.5 Hz), 5.81 (1H, s), 7.11 (1H, d, J=10 Hz), 7.14-7.23 (3H, m), 7.24-7.32 (2H, m), 7.38-7.48 (3H, m), 7.52-7.60 (2H, m), 8.00-8.06 (2H, m);

MASS (ES−): m/e 589.

Preparation 282

Compound (282) was obtained in a manner similar to Preparation 77.

¹H-NMR (300 MHz, CDCl₃, δ): 0.87 (3H, t, J=7 Hz), 1.28 (3H, s), 1.30-1.70 (5H, m), 1.75-1.92 (3H, m), 2.00-2.42 (6H, m), 2.64 (2H, m), 3.32 (1H, m), 3.65 (2H, brt, J=6 Hz), 3.74 (1H, m), 4.22 (1H, dt, J=10, 7.5. Hz), 4.72 (1H, m), 4.84 (1H, dt, J=10, 7.5 Hz), 5.91 (1H, s), 7.10 (1H, d, J=10 Hz), 7.14-7.23 (3H, m), 7.24-7.33 (2H, m), 7.41 (1H, d, J=10 Hz);

MASS (ES−): m/e 485.

Preparation 283

Compound (283) was obtained in a manner similar to Preparation 78. The obtained compound was used in Examples 132, 135.

¹H-NMR (300 MHz, CDCl₃, δ): 0.87 (3H, t, J=7.5 Hz), 1.29 (3H, s), 1.58-1.73 (2H, m), 1.76-1.91 (3H, m), 1.98-2.24 (5H, m), 2.26-2.42 (3H, m), 2.50 (2H, m), 2.64 (2H, m), 3.32 (1H, m), 3.75 (1H, m), 4.23 (1H, m), 4.72 (1H, m), 4.84 (1H, ddd, J=10, 8, 7 Hz), 5.85 (1H, s), 7.12 (1H, d, J=10.5 Hz), 7.14-7.32 (5H, m), 7.36 (1H, d, J=10 Hz), 9.77 (1H, t, J=1 Hz);

MASS (ES−): m/e 483.

Preparation 284

Compound (284) was obtained in a manner similar to Preparation 14.

¹H-NMR (300 MHz, CDCl₃, δ): 1.49 (9H, s), 1.51-1.63 (1H, m), 1.74-2.01 (3H, m), 2.62-2.80 (1H, m), 2.90 (1H, dd, J=12.5, 9.6 Hz), 3.01 (1H, dd, J=12.5, 5.6 Hz), 3.48-3.66 (1H, m), 4.27 (1H, t, J=7.0 Hz), 4.35 (1H, dd, J=8.0, 3.7 Hz), 4.55 (2H, d, J=7.0 Hz), 4.56-4.67 (1H, m), 5.11 (1H, d, J=12.4 Hz), 5.21 (1H, d, J=12.4 Hz), 5.37 (1H, d, J=8.5 Hz), 6.62 (1H, brs), 7.07-7.49 (13H, m), 7.62 (2H, d, J=7.3 Hz), 7.79 (2H, d, J=7.8 Hz);

MASS (ES+): m/e 690.49 (M+1).

Preparation 285

Compound (285) was obtained in a manner similar to Preparation 15.

¹H-NMR (300 MHz, CDCl₃, δ): 0.80 (3H, t, J=7.3 Hz), 1.39 (1.5H, s), 1.40 (1.5H, s), 1.43 (9H, s), 1.49-1.65 (2H, m), 1.71-2.07 (4H, m), 2.69-2.85 (1H, m), 2.91 (1H, dd, J=12.9, 9.2 Hz), 3.01 (1H, dd, J=12.9, 5.4 Hz), 3.49-3.62 (1H, m), 4.27 (1H, t, J=6.6 Hz), 4.37 (1H, dd, J=7.8, 3.4 Hz), 4.54 (2H, t, J=6.6 Hz), 4.85-4.98 (1H, m), 5.01-5.20 (3H, m), 6.52-6.67 (1H, m), 6.84 (1H, d, J=8.1 Hz), 7.10-7.19 (2H, m), 7.20-7.38 (9H, m), 7.42 (2H, t, J=7.3 Hz), 7.61 (2H, t, J=7.4 Hz), 7.79 (2H, t, J=7.4 Hz);

MASS (ES+): m/e 789.65 (M+1).

Preparation 286

Compound (286) was obtained in a manner similar to Preparation 16.

¹H-NMR (300 MHz, CDCl₃, δ): 0.72 (3H, t, J=7.3 Hz), 1.43 (3H, s), 1.44 (9H, s), 1.45-1.98 (9H, m), 2.15-2.36 (1H, m), 2.74-3.03 (3H, m), 3.52-3.66 (1H, m), 4.20-4.34 (3H, m), 4.39 (1H, dd, J=7.8, 3.5 Hz), 4.52 (2H, t, J=6.6 Hz), 4.85-4.99 (1H, m), 5.01-5.21 (3H, m), 6.61-6.84 (2H, m), 6.98 (1H, s), 7.11 (2H, d, J=8.4 Hz), 7.20-7.36 (1H, m), 7.41 (2H, t, J=7.7 Hz), 7.50-7.58 (1H, m), 7.61 (2H, d, J=7.3 Hz), 7.78 (2H, d, J=7.3 Hz), 8.03 (2H, d, J=6.9 Hz).

Preparation 287

Compound (287) was obtained in a manner similar to Preparation 17.

¹H-NMR (300 MHz, CDCl₃, δ): 0.76 (3H, t, J=7.3 Hz), 1.37-3.01 (15H, m), 1.44 (12H, s), 3.61-3.75 (1H, m), 3.94-4.08 (1H, m), 4.22-4.40 (4H, m), 4.54 (2H, brd, J=6.6 Hz), 4.83-4.98 (1H, m), 5.24 (1H, brs), 6.60 (0.4H, brd, J=8.4 Hz), 6.67 (1H, brs), 6.84 (1H, brs), 6.98 (0.6H, brd, J=8.1 Hz), 7.14 (2H, brd, J=8.1 Hz), 7.21-7.47 (6H, m), 7.48-7.66 (3H, m), 7.71-7.82 (2H, m), 7.99-8.08 (2H, m);

MASS (ES+): m/e 932.42 (M+1).

Preparation 288

Compound (288) was obtained in a manner similar to Preparation 18.

¹H-NMR (300 MHz, CDCl₃, δ): 0.71 (3H, brs), 1.38 (3H, brs), 1.47-2.22 (12H, m), 2.74-3.19 (3H, m), 3.56-3.81 (1H, m), 4.08-4.51 (6H, m), 4.82-5.04 (1H, m), 7.02-7.16 (2H, m), 7.17-7.43 (9H, m), 7.44-7.67 (4H, m), 7.69-7.81 (2H, m), 7.91-8.05 (2H, m), 8.11-8.35 (2H, m), 8.37-8.62 (1H, m);

MASS (ES+): m/e-832.64 (M+1).

Preparation 289

Compound (289) was obtained in a manner similar to Preparation 76.

¹H-NMR (300 MHz, CDCl₃, δ): 0.82 (3H, t, J=7.3 Hz), 1.27 (3H, s), 1.35-2.02 (8H, m), 2.06-2.24 (2H, m), 2.25-2.41 (2H, m), 2.91 (1H, dd, J=13.6, 6.2 Hz), 3.08-3.32 (2H, m), 3.79-3.92 (1H, m), 4.18-4.30 (2H, m), 4.31 (2H, t, J=6.3 Hz), 4.54 (2H, d, J=6.6 Hz), 4.66 (1H, brd, J=7.0 Hz), 5.14 (1H, dt, J=10.2, 6.3 Hz), 5.90 (1H, s), 6.63 (1H, brs), 7.13 (1H, d, J=10.6 Hz), 7.16 (2H, d, J=8.8 Hz), 7.23-7.37 (4H, m), 7.38-7.48 (4H, m), 7.51-7.65 (4H, m), 7.78 (2H, d, J=7.3 Hz), 8.00-8.07 (2H, m);

MASS (ES+): m/e 813.89 (M).

Preparation 290

Compound (290) was obtained in a manner similar to Preparation 21.

¹H-NMR (300 MHz, DMSO-d₆, δ): 1.52 (1H, m), 1.66-1.86 (2H, m), 1.94 (1H, m), 2.72 (1H, m), 2.97 (1H, dd, J=13.5, 8.5 Hz), 3.14 (1H, dd, J=13.5, 6 Hz), 3.56 (1H, m), 4.28 (1H, dd, J=9, 3.5 Hz), 4.41 (1H, brdd, J=8.5, 6 Hz), 5.08 (1H, d, J=12.5 Hz), 5.19 (1H, d, J=12.5 Hz), 7.20-7.43 (10H, m), 8.40 (2H, brs);

MASS (ES+): m/e 353.

Preparation 291

Compound (291) was obtained in a manner similar to Preparation 22.

¹H-NMR (300 MHz, CDCl₃, δ): 1.40 (3×3H, s), 1.47 (1H, m), 1.58-1.94 (3H, m), 2.56 (1H, m), 2.77 (1H, dd, J=13, 10 Hz), 2.83-3.08 (3H, m), 3.48 (1H, m), 3.76 (3H, s), 4.32 (1H, dd, J=8, 4 Hz), 4.84-5.02 (2H, m), 5.10 (1H, d, J=12.5 Hz), 5.17 (1H, d, J=12.5 Hz), 6.67 (1H, d, J=8 Hz), 6.83 (2×1H, d, J=8 Hz), 6.98-7.40 (11H, m), 7.09 (2×1H, d, J=8 Hz);

MASS (ES+): m/e 630.

Preparation 292

Compound (292) was obtained in a manner similar to Preparation 16.

¹H-NMR (300 MHz, DMSO-d₆, δ): 1.70-2.30 (4H, m), 2.41-2.98 (4H, m), 3.26-3.76 (2H, m), 3.70 (3×⅕H, s), 3.71 (3×⅘H, s), 3.83-4.01 (2H, m), 4.32 (1×⅘H, dd, J=8, 3 Hz), 4.44 (1×⅕H, m), 4.88 (1×⅘H, m), 5.06 (1×⅕H, m), 5.10 (1×⅘H, d, J=12.5 Hz), 5.14 (1×⅘H, d, J=12.5 Hz), 5.21 (1×⅕H, d, J=12.5 Hz), 5.31 (1×⅕H, d, J=12.5 Hz), 6.67-6.78 (4×⅕H, m), 6.84 (2×⅘H, d, J=9 Hz), 7.02 (2×⅘H, d, J=9 Hz), 7.08-7.44 (10H, m), 8.07 (2H, br), 9.00 (1×⅘H, d, J=8 Hz), 9.26 (1×⅕H, d, J=8 Hz);

MASS (ES+): m/e 530.

Preparation 293

Compound (293) was obtained in a manner similar to Preparation 17.

¹H-NMR (300 MHz, CDCl₃, δ): 0.82 (3H, t, J=7.3 Hz), 1.28 (3H, s), 1.36-1.56 (2H, m), 1.62-1.98 (6H, m), 2.06-2.40 (4H, m), 2.88 (1H, dd, J=13.5, 6 Hz), 3.16 (1H, dd, J=13.5, 9.5 Hz), 3.26 (1H, m), 3.84 (1H, m), 4.24 (1H, dt, J=10, 7.5 Hz), 4.31 (2H, t, J=6.5 Hz), 4.68 (1H, dd, J=8, 2.5 Hz), 5.12 (1H, ddd, J=10, 9.5, 6 Hz), 5.46 (2H, s), 5.90 (1H, s), 6.73 (2×1H, d, J=8.3 Hz), 7.08 (2×1H, d, J=8.3 Hz), 7.14 (1H, d, J=10 Hz), 7.43 (2×1H, dd, J=7.5, 7.5 Hz), 7.52-7.60 (2H, m), 8.03 (2×1H, dd, J=7.5, 1.5 Hz);

MASS (ES+): m/e 593.38.

Preparation 294

The Compound (293) was dissolved in dichloromethane (30 ml), ethylisopropylamine (1.76 ml) was added to the mixture. To the mixture was added N-phenylbis(trifluoromethanesulfonimide) (manufactured by Tokyo Kasei Kogyo Co., Ltd., 1.27 g), and the mixture was stirred under ambient temperature overnight. The solvent was removed by evaporation. The residue was dissolved in ethyl acetate, washed with 5% aqueous potassium hydrogensulfate (×2), saturated aqueous sodium bicarbonate solution and saturated brine, dried over sodium sulfate and evaporated. The residue was purified by flush chromatography (Silica gel 60N, Spherical, 45 g, eluting with ethyl acetate/hexane=1/1 then 2/1) to give the objective Compound (294) as a white foam.

¹H-NMR (300 MHz, CDCl₃, δ): 0.79 (3H, t, J=7.5 Hz), 1.27 (3H, s), 1.38-1.54 (2H, m), 1.66-1.98 (6H, m), 2.06-2.40 (4H, m), 3.02 (1H, dd, J=13.5, 6.5 Hz), 3.25 (1H, dd, J=13.5, 9.5 Hz), 3.28 (1H, m), 3.84 (1H, m), 4.25 (1H, dt, J=10, 7.5 Hz), 4.32 (2×1H, t, J=6.5 Hz), 4.69 (1H, dd, J=8, 2 Hz), 5.16 (1H, ddd, J=10.3, 9.5, 6.5 Hz), 5.92 (1H, s), 7.05 (1H, d, J=10 Hz), 7.19 (2×1H, d, J=8.7 Hz), 7.32 (2×1H, d, J=8.7 Hz), 7.44 (2×1H, dd, J=7.5, 7.5 Hz), 7.56 (1H, m), 8.03 (2×1H, dd, J=7.5, 1.5 Hz);

MASS (ES+): m/e 725.42.

Preparation 295

To a solution of the Compound (294) (1.4 g) in N,N-dimethylformamide (28 ml) was added lithium chloride (573 mg) and dichlorobis(trichlorophosphine)palladium (II) (67.8 mg), and the mixture was degassed with ultrasonic for 2 min. After purging the air from the reaction vessel with nitrogen, the mixture was stirred at 100° C. overnight. The reaction mixture was cooled to ambient temperature, and an aqueous potassium fluoride (2 g/10 ml) was added and the mixture was stirred for 30 min. The reaction mixture was washed with ethyl acetate and the insoluble-matter in the mixture was filtered off. The mixture was purified by silica gel column chromatography (eluting with hexane/ethyl acetate=1/4, ethyl acetate, then hexane/ethyl acetate=9/1) to give the objective Compound (295).

¹H-NMR (300 MHz, CDCl₃, δ): 0.83 (3H, t, J=7.3 Hz), 1.11-2.00 (8H, m), 1.28 (3H, s), 2.07-2.24 (2H, m), 2.24-2.43 (2H, m), 2.97-3.07 (1H, m), 3.20-3.37 (2H, m), 3.81-3.94 (1H, m), 4.18-4.30 (1H, m), 4.32 (2H, t, J=6.3 Hz), 4.62-4.70 (1H, m), 5.16-5.28 (1H, m), 5.86 (1H, s), 7.14 (1H, d, J=9.9 Hz), 7.19-7.31 (1H, m), 7.35 (2H, d, J=8.4 Hz), 7.40-7.81 (6H, m), 7.90 (2H, d, J=8.4 Hz), 8.03 (2H, d, J=8.4 Hz), 8.65-8.71 (1H, m);

MASS (ES+): m/e 654.28 (M+1).

Preparation 296

Compound (296) was obtained in a manner similar to Preparation 77.

¹H-NMR (300 MHz, CDCl₃, δ): 0.86 (3H, s), 1.16-1.95 (8H, m), 1.29 (3H, s), 2.08-2.25 (2H, m), 2.25-2.42 (2H, m), 3.02 (1H, dd, J=13.6, 6.2 Hz), 3.20-3.36 (1H, m), 3.32 (1H, dd, J=13.6, 9.9 Hz), 3.81-3.93 (1H, m), 4.18-4.29 (1H, m), 4.64-4.73 (1H, m), 5.24 (1H, ddd, J=10.3, 9.9, 6.2 Hz), 5.99 (1H, s), 7.15 (1H, d, J=10.3 Hz), 7.20-7.32 (1H, m), 7.35 (2H, d, J=8.4 Hz), 7.60 (1H, d, J=10.3 Hz), 7.67-7.80 (2H, m), 7.91 (2H, d, J=8.4 Hz), 8.66-8.72 (1H, m);

MASS (ES+): m/e 550.39 (M+1).

Preparation 297

Compound (297) was obtained in a manner similar to Preparation 78. The obtained compound was used in Example 150.

¹H-NMR (300 MHz, CDCl₃, δ): 0.86 (3H, t, J=7.3 Hz), 1.30 (3H, s), 1.38-1.94 (6H, m), 2.09-2.24 (2H, m), 2.24-2.41° (2H, m), 2.50 (2H, t, J=6.5 Hz), 3.01 (1H, dd, J=13.6, 5.9 Hz), 3.19-3.33 (1H, m), 3.31 (1H, dd, J=13.6, 10.6 Hz), 3.82-3.93 (1H, m), 4.18-4.29 (1H, m), 4.63-4.71 (1H, m), 5.23 (1H, ddd, J=10.6, 10.3, 5.9 Hz), 5.94 (1H, s), 7.16 (1H, d, J=9.9 Hz), 7.19-7.30 (1H, m), 7.35 (2H, d, J=8.4 Hz), 7.54 (1H, d, J=10.3 Hz), 7.66-7.79 (2H, m), 7.90 (2H, d, J=8.4 Hz), 8.65-8.69 (1H, m), 9.77 (1H, s);

MASS (ES+): m/e 548.30 (M+1).

Preparation 298

Compound (298) was obtained in a manner similar to Preparation 295.

¹H-NMR (300 MHz, CDCl₃, δ): 0.82 (3H, t, J=7.3 Hz), 1.29 (3H, s), 1.34-2.01 (8H, m), 2.07-2.41 (4H, m), 3.05 (1H, dd, J=13.9, 6.6 Hz), 3.23-3.39 (1H, m), 3.31 (1H, dd, J=13.9, 9.2 Hz), 3.82-3.94 (1H, m), 4.20-4.37 (1H, m), 4.33 (2H, t, J=6.6 Hz), 4.66-4.74 (1H, m), 5.24 (1H, ddd, J=10.6, 9.2, 6.6 Hz), 5.96 (1H, s), 7.12 (1H, d, J=10.6 Hz), 7.36 (2H, d, J=8.1 Hz), 7.40-7.72 (5H, m), 7.58 (2H, d, J=8.1 Hz), 7.63 (1H, d, J=10.6 Hz), 8.04 (2H, d, J=8.4 Hz), 8.64 (2H, d, J=5.9 Hz);

MASS (ES+): m/e 654.48 (M+1).

Preparation 299

Compound (299) was obtained in a manner similar to Preparation 77.

¹H-NMR (300 MHz, CDCl₃, δ): 0.83 (3H, t, J=7.3 Hz), 1.19-1.96 (8H, m), 1.29 (3H, s), 2.05-2.40 (4H, m), 3.04 (1H, dd, J=13.9, 6.6 Hz), 3.26-3.38 (1H, m), 3.30 (1H, dd, J=13.9, 9.2 Hz), 3.66 (2H, t, J=6.3 Hz), 3.82-3.93 (1H, m), 4.24 (1H, dt, J=10.3, 7.7 Hz), 4.66-4.73 (1H, m), 5.23 (1H, s), 6.00 (1H, s), 7.10 (1H, d, J=10.3 Hz), 7.35 (2H, d, J=8.1 Hz), 7.49 (2H, d, J=5.9 Hz), 7.57 (2H, d, J=8.1 Hz), 7.62 (1H, d, J=10.3 Hz), 8.64 (2H, d, J=5.9 Hz);

MASS (ES+): m/e 550.33 (M+1).

Preparation 300

Compound (300) was obtained in a manner similar to Preparation 78. The obtained compound was used in Example 153.

¹H-NMR (300 MHz, CDCl₃, δ): 0.84 (3H, t), 1.30 (3H, s), 1.46-1.92 (6H, m), 2.10-2.39 (4H, m), 2.50 (2H, t, J=6.2 Hz), 3.04 (1H, dd, J=13.9, 6.6 Hz), 3.21-3.38 (1H, m), 3.29 (1H, dd, J=13.9, 9.5 Hz), 3.83-3.94 (1H, m), 4.25 (1H, dt, J=10.3, 7.3 Hz), 4.65-4.74 (1H, m), 5.22 (1H, ddd, J=10.6, 9.5, 6.6 Hz), 5.99 (1H, s), 7.12 (1H, d, J=10.3 Hz), 7.36 (2H, d, J=8.4 Hz), 7.49 (2H, dd, J=4.4, 1.5 Hz), 7.56 (1H, d, J=10.6 Hz), 7.57 (2H, d, J=8.4 Hz), 8.64 (2H, dd, J=4.4, 1.5 Hz), 9.78 (1H, s);

MASS (ES+): m/e 548.28 (M+1).

Preparation 301

To a solution of the Compound (293) (4.02 g) in acetone (2 ml) was added t-butoxycarbonylmethyl bromide (2.65 g) and potassium carbonate (4.69 g), and the mixture was stirred at 50° C. for 4 hours. The reaction mixture was extracted with ethyl acetate, washed with 5% potassium hydrogensulfate (×2), saturated aqueous sodium bicarbonate solution, water and brine, and dried over sodium sulfate. The mixture was purified by silica gel column chromatography (eluting with hexane/ethyl acetate=1/2) to give the objective Compound (301).

¹H-NMR (300 MHz, CDCl₃, δ): 0.82 (3H, t, J=7.3 Hz), 1.28 (3H, s), 1.36-1.56 (2H, m), 1.48 (9H, s), 1.58-1.96 (6H, m), 2.07-2.24 (2H, m), 2.24-2.40 (2H, m), 2.89 (1H, dd, J=13.6, 5.9 Hz), 3.16-3.30 (1H, m), 3.18 (1H, dd, J=13.6, 9.6 Hz), 3.80-3.90 (1H, m), 4.24 (1H, dt, J=10.3, 7.7 Hz), 4.32 (2H, t, J=6.6 Hz), 4.47 (2H, s), 4.63-4.69 (1H, m), 5.13 (1H, ddd, J=10.3, 9.6, 5.9 Hz), 5.89 (1H, s), 6.80 (2H, d, J=8.8 Hz), 7.12-7.18 (1H, m), 7.14 (2H, d, J=8.8 Hz), 7.40-7.48 (2H, m), 7.50-7.59 (2H, m), 8.03 (2H, d, J=8.4 Hz);

MASS (ES+): m/e 707.53 (M+1).

Preparation 302

To a solution of the Compound (301) (500 mg) in methylene chloride (6 ml) was added trifluoroacetic acid (2 ml) and the mixture was stirred at ambient temperature for 2.5 hours. The solvent was evaporated in vacuo to give the objective Compound (302).

¹H-NMR (300 MHz, CDCl₃, δ): 0.81 (3H, t, J#7.3 Hz), 1.19-1.56 (4H, m), 1.27 (3H, s), 1.60-1.97 (4H, m), 2.03-2.23 (2H, m), 2.23-2.39 (2H, m), 2.87 (1H, dd, J=13.9, 6.2 Hz), 3.14 (1H, dd, J=13.9, 9.5 Hz), 3.15-3.30 (1H, m), 3.62-3.89 (2H, m), 4.25 (1H, dt, J=10.5, 7.3 Hz), 4.32 (2H, t, J=6.6 Hz), 4.62-4.71 (1H, m), 4.65 (2H, s), 5.12 (1H, ddd, J=10.3, 9.5, 6.2 Hz), 6.15 (1H, s), 6.83 (2H, d, J=8.4 Hz), 7.14 (2H, d, J=8.4 Hz), 7.25 (1H, d, J=10.3 Hz), 7.40-7.48 (2H, m), 7.52-7.60 (1H, m), 7.64 (1H, d, J=10.3 Hz), 8.03 (2H, d, J=8.4 Hz);

MASS (ES+): m/e 651.51 (M+1).

Preparation 303

To a solution of the Compound (302) (405 mg) in N,N-dimethylformamide (4 ml) was added PyBOP (357 mg) and diisopropylethylamine (178 mg), and the mixture was stirred. To the mixture was added N-morpholine (81.6 mg) and the mixture was stirred at ambient temperature for 1.5 hour. The reaction mixture was extracted with ethyl acetate, washed with a 5% aqueous potassium hydrogensulfate solution (×2), saturated aqueous sodium bicarbonate solution (×2), water and brine, and dried over sodium sulfate. The mixture was purified by silica gel column chromatography (eluting with ethyl acetate then ethyl acetate/methanol=9/1) to give the object Compound (303).

¹H-NMR (300 MHz, CDCl₃, δ): 0.82 (3H, t, J=7.3 Hz), 1.27 (3H, S), 1.35-1.98 (8H, m), 2.08-2.39 (4H, m), 2.58 (2H, t, J=7.3 Hz), 2.87-2.99 (3H, m), 3.20 (1H, dd, J=13.5, 9.2 Hz), 3.24-3.38 (3H, m), 3.46-3.55 (2H, m), 3.56-3.66 (4H, m), 3.83-3.93 (1H, m), 4.25 (1H, dt, J=10.3, 7.7 Hz), 4.32 (2H, t, J=6.2 Hz), 4.65-4.71 (1H, m), 5.11-5.22 (1H, m), 5.87 (1H, S), 7.09-7.19 (1H, m), 7.11 (2H, d, J=8.4 Hz), 7.16 (2H, d, J=8.4 Hz), 7.40-7.49 (2H, m), 7.53-7.60 (2H, m), 8.03 (2H, d, J=8.4 Hz);

MASS (ES+): m/e 718.52 (M+1).

Preparation 304

Compound (304) was obtained in a manner similar to Preparation 77.

¹H-NMR (300 MHz, CDCl₃, δ): 0.84 (3H, t, J=7.3 Hz), 1.22-1.94 (8H, m), 1.28 (3H, s), 2.08-2.39 (4H, m), 2.58 (2H, t, J=7.3 Hz), 2.88-2.98 (2H, m), 2.94 (1H, dd, J=13.9, 6.6 Hz), 3.20 (1H, dd, J=13.9, 9.5 Hz), 3.25-3.38 (3H, m), 3.47-3.55 (2H, m), 3.56-3.69 (4H, m), 3.65 (2H, t, J=6.2 Hz), 3.80-3.93 (1H, m), 4.23 (1H, dt, J=10.3, 8.1 Hz), 4.66-4.71 (1H, m), 5.11-5.23 (1H, m), 5.96 (1H, s), 7.05-7.20 (1H, m), 7.11 (2H, d, J=8.1 Hz), 7.16 (2H, d, J=8.1 Hz), 7.56 (1H, d, J=10.3 Hz);

MASS (ES+): m/e 614.55 (M+1).

Preparation 305

Compound (305) was obtained in a manner similar to Preparation 78. The obtained compound was used in Example 156.

¹H-NMR (300 MHz, CDCl₃, δ): 0.84 (3H, t, J=7.3 Hz), 1.29 (3H, s), 1.46-1.92 (6H, m), 2.08-2.38 (4H, m), 2.45-2.54 (2H, m), 2.58 (2H, t, J=7.3 Hz), 2.87-2.98 (3H, m), 3.20 (1H, dd, J=13.5, 9.5 Hz), 3.23-3.39 (3H, m), 3.45-3.55 (2H, m), 3.56-3.67 (4H, m), 3.82-3.93 (1H, m), 4.24 (1H, dt, J=10.3, 7.0 Hz), 4.64-4.72 (1H, m), 5.17 (1H, ddd, J=9.9, 9.5, 6.6 Hz), 5.92 (1H, s), 7.08-7.19 (5H, m), 7.51 (1H, d, J=10.3 Hz), 9.77 (1H, s);

MASS (ES+): m/e 612.56 (M+1).

Preparation 306

Compound (306) was obtained in a manner similar to Preparation 303.

¹H-NMR (300 MHz, CDCl₃, δ): 0.82 (3H, t, J=7.3 Hz), 1.28 (12H, s), 1.35-1.98 (8H, m), 2.06-2.36 (4H, m), 2.34 (2H, t, J=7.7 Hz), 2.89 (2H, t, J=7.7 Hz), 2.92 (1H, dd, J=13.5, 6.2 Hz), 3.21 (1H, dd, J=13.5, 9.9 Hz), 3.22-3.33 (1H, m), 3.81-3.92 (1H, m), 4.24 (1H, dt, J=10.6, 7.7 Hz), 4.32 (2H, t, J=6.6 Hz), 4.64-4.71 (1H, m), 5.10 (1H, s), 5.16 (1H, ddd, J=9.9, 9.9, 5.9 Hz), 5.85 (1H, s), 7.07-7.20 (1H, m), 7.09 (2H, d, J=8.4 Hz), 7.14 (2H, d, J=8.4 Hz), 7.40-7.49 (2H, m), 7.51-7.60 (2H, m), 8.03 (2H, d, J=8.4 Hz);

MASS (ES+): m/e 704.53 (M+1).

Preparation 307

Compound (307) was obtained in a manner similar to Preparation 77.

¹H-NMR (300 MHz, CDCl₃, δ): 0.84 (3H, t, J=7.3 Hz), 1.23-1.90 (8H, m), 1.28 (12H, s), 2.10-2.37 (4H, m), 2.58 (2H, t, J=8.4 Hz), 2.93 (2H, t, J=8.4 Hz), 2.95 (1H, dd, J=13.9, 6.2 Hz), 3.20 (1H, dd, J=13.9, 9.5 Hz), 3.27-3.39 (3H, m), 3.47-3.55 (2H, m), 3.56-3.69 (4H, m), 3.66 (2H, t, J=6.2 Hz), 3.82-3.93 (1H, m), 4.23 (1H, dt, J=10.3, 7.7 Hz), 4.66-4.71 (1H, m), 5.17 (1H, ddd, J=10.3, 9.5, 6.6 Hz), 5.96 (1H, s), 7.06-7.18 (1H, m), 7.11 (2H, d, J=8.1 Hz), 7.16 (2H, d, J=8.1 Hz), 7.55 (1H, d, J=10.3 Hz);

MASS (ES+): m/e 600.57 (M+1).

Preparation 308

Compound (308) was obtained in a manner similar to Preparation 78. The obtained compound was used in Example 159.

¹H-NMR (300 MHz, CDCl₃, δ): 0.84 (3H, t, J=7.3 Hz), 1.28 (12H, s), 1.42-1.90 (6H, m), 2.09-2.40 (4H, m), 2.34 (2H, t, J=7.3 Hz), 2.46-2.61 (2H, m), 2.88 (2H, t, J=7.3 Hz), 2.93 (1H, dd, J=13.9, 6.2 Hz), 3.20 (1H, dd, J=13.9, 9.2 Hz), 3.22-3.38 (1H, m), 3.82-3.92 (1H, m), 4.23 (1H, dt, J=9.9, 7.3 Hz), 4.64-4.71 (1H, m), 5.10 (1H, s), 5.16 (1H, ddd, J=10.6, 9.2, 6.2 Hz), 5.91 (1H, s), 7.07-7.18 (1H, m), 7.10 (2H, d, J=8.1 Hz), 7.14 (2H, d, J=8.1 Hz), 7.49 (1H, d, J=9.9 Hz), 9.77 (1H, s);

MASS (ES+): m/e 598.59 (M+1).

Preparation 309

To a solution of the Compound (293) (148 mg) in a mixed solvent of carbon tetrachloride/acetonitrile/water (0.4, 0.4 and 0.6 ml) was added sodium periodate (758 mg) and the mixture was stirred. To the mixture was added ruthenium(IV) oxide catalyst (0.665 mg) and the mixture was stirred at ambient temperature for 36 hours. To the reaction mixture was added ethyl acetate and the insoluble matter was filtered off. The mixture was extracted with water and ethyl acetate, and the organic layer was evaporated. The residue was purified by preparative chromatography (chloroform/methanol=9/1) to give the objective Compound (309).

¹H-NMR (300 MHz, CDCl₃, δ): 0.83 (3H, t, J=7.3 Hz), 1.29 (3H, s), 1.34-2.44 (12H, m), 2.65 (1H, dd, J=17.2, 3.7 Hz), 3.18 (1H, dd, J=17.2, 10.3 Hz), 3.71 (1H, dt, J=9.9, 7.0 Hz), 3.84-3.95 (1H, m), 4.26 (1H, dt, J=10.3, 7.7 Hz), 4.31 (2H, t, J=6.6 Hz), 4.69-4.76 (1H, m), 5.28 (1H, ddd, J=10.3, 10.3, 3.7 Hz), 5.97 (1H, s), 7.08 (1H, d, J=10.3 Hz), 7.40-7.48 (1H, m), 7.52-7.60 (1H, m), 7.60 (1H, d, J=10.3 Hz), 8.03 (2H, d, J=8.4 Hz);

MASS (ES+): m/e 545.49 (M+1).

Preparation 310

Compound (310) was obtained in a manner similar to Preparation 303.

¹H-NMR (300 MHz, CDCl₃, δ): 0.84 (3H, t, J=7.3 Hz), 1.34-2.42 (12H, m), 1.30 (3H, S), 2.72 (1H, dd, J=15.4, 4.4 Hz), 3.21 (1H, dd, J=15.4, 10.9 Hz), 3.81 (1H, dt, J=10.3, 7.7 Hz), 3.92 (1H, dt, J=10.3, 4.8 Hz), 4.26 (1H, dt, J=9.9, 8.1 Hz), 4.31 (2H, t, J=6.6 Hz), 4.68-4.74 (1H, m), 5.44 (1H, ddd, J=10.9, 10.6, 4.4 Hz), 5.94 (1H, s), 7.09 (1H, d, J=10.6 Hz), 7.11 (1H, dd, J=7.7, 7.7 Hz), 7.30 (2H, dd, J=8.1, 7.7 Hz), 7.39-7.47 (4H, m), 7.50 (1H, d, J=9.9 Hz), 7.51-7.61 (2H, m), 8.03 (2H, d, J=8.4 Hz);

MASS (ES+): m/e 620.51 (M+1).

Preparation 311

Compound (311) was obtained in a manner similar to Preparation 77.

¹H-NMR (300 MHz, CDCl₃, δ): 0.86 (3H, t, J=7.3 Hz), 1.18-2.43 (12H, m), 1.31 (9H, s), 2.73 (1H, dd, J=15.4, 4.4 Hz), 3.20 (1H, dd, J=15.4, 10.6 Hz), 3.65 (2H, t, J=6.2 Hz), 3.82 (1H, dt, J=10.3, 7.7 Hz), 3.92 (1H, dt, J=10.3, 4.4 Hz), 4.25 (1H, dt, J=10.3, 7.7 Hz), 4.68-4.76 (1H, m), 5.44 (1H, ddd, J=11.0, 10.6, 4.4 Hz), 6.12 (1H, s), 7.10 (1H, dd, J=7.3, 7.3 Hz), 7.11 (1H, d, J=10.3 Hz), 7.30 (2H, dd, J=7.7, 7.3 Hz), 7.44 (2H, d, J=7.7 Hz), 7.51 (1H, d, J=11.0 Hz), 7.55-7.65 (1H, m);

MASS (ES+): m/e 516.56 (M+1).

Preparation 312

Compound (312) was obtained in a manner similar to Preparation 78. The obtained compound was used in Example 166.

¹H-NMR (300 MHz, CDCl₃, δ): 0.86 (3H, t, J=7.3 Hz), 1.31 (3H, S), 1.35-2.42 (10H, m), 2.49 (2H, t, J=7.0 Hz), 2.72 (1H, dd, J=15.4, 4.4 Hz), 3.20 (1H, dd, J=15.4, 11.0 Hz), 3.81 (1H, dt, J=10.3, 7.0 Hz), 3.93 (1H, dt, J=10.3, 5.1 Hz), 4.25 (1H, dt, J=9.9, 7.7 Hz), 4.67-4.74 (1H, m), 5.44 (1H, ddd, J=11.0, 10.6, 4.4 Hz), 5.98 (1H, S), 7.05-7.14 (2H, m), 7.30 (2H, dd, J=8.1, 7.7 Hz), 7.38-7.49 (2H, m), 7.44 (2H, d, J=8.1 Hz), 9.76 (1H, S);

MASS (ES+): m/e 514.52 (M+1).

Preparation 313

Compound (313) was obtained in a manner similar to Preparation 303.

¹H-NMR (300 MHz, CDCl₃, δ): 0.81 (3H, t, J=7.3 Hz), 1.27 (3H, s), 1.38-1.98 (8H, m), 2.07-2.38 (4H, m), 2.93 (1H, dd, J=13.9, 6.2 Hz), 3.20 (1H, dd, J=13.9, 9.5 Hz), 3.22-3.34 (1H, m), 3.81-3.91 (1H, m), 4.25 (1H, dt, J=10.3, 7.3 Hz), 4.32 (2H, t, J=6.6 Hz), 4.58 (2H, S), 4.64-4.71 (1H, m), 5.14 (1H, ddd, J=10.3, 9.5, 6.2 Hz), 5.88 (1H, s), 6.91 (2H, d, J=8.4 Hz), 7.11 (1H, d, J=9.9 Hz), 7.16 (1H, dd, J=7.7, 7.7. Hz), 7.21 (2H, d, J=8.4 Hz), 7.36 (2H, dd, J=7.7, 7.3 Hz), 7.40-7.49 (2H, m), 7.53-7.62 (2H, m), 7.58 (2H, d, J=7.3 Hz), 8.03 (2H, d, J=8.4 Hz), 8.24 (1H, brs);

MASS (ES+): m/e 726.52 (M+1).

Preparation 314

Compound (314) was obtained in a manner similar to Preparation 77.

¹H-NMR (300 MHz, CDCl₃, δ): 0.83 (3H, t), 1.18-1.92 (8H, m), 1.28 (3H, S), 2.08-2.39 (4H, m), 2.93 (1H, dd, J=13.9, 6.6 Hz), 3.19 (1H, dd, J=13.9, 9.2 Hz), 3.21-3.34 (1H, m), 3.60-3.70 (2H, m), 3.80-3.90 (1H, m), 4.23 (1H, dt, J=10.3, 7.7 Hz), 4.58 (2H, S), 4.64-4.71 (1H, m), 5.14 (1H, ddd, J=9.9, 9.2, 6.6 Hz), 5.90 (1H, S), 6.91 (2H, d, J=8.4 Hz), 7.09 (1H, d, J=10.3 Hz), 7.16 (2H, dd, J=7.3, 7.3 Hz), 7.21 (2H, d, J=8.4 Hz), 7.36 (2H, dd, J=7.3, 7.3 Hz), 7.55 (1H, d, J=9.9 Hz), 7.57 (2H, d, J=7.3 Hz), 8.24 (1H, brs);

MASS (ES+): m/e 622.54 (M+1).

Preparation 315

Compound (315) was obtained in a manner similar to Preparation 78. The obtained compound was used in Example 169.

¹H-NMR (300 MHz, CDCl₃, δ): 0.83 (3H, t, J=7.3 Hz), 1.29 (3H, S), 1.48-1.91 (6H, m), 2.09-2.39 (4H, m), 2.45-2.54 (2H, m), 2.93 (1H, dd, J=13.5, 6.6 Hz), 3.19 (1H, dd, J=13.5, 9.6 Hz), 3.22-3.33 (1H, m), 3.81-3.91 (1H, m), 4.23 (1H, dt, J=10.6, 7.3 Hz), 4.64-4.72 (1H, m), 5.14 (1H, ddd, J=9.9, 9.6, 6.6 Hz), 5.89 (1H, S), 6.91 (2H, d, J=8.4 Hz), 7.12 (1H, d, J=10.6 Hz), 7.15 (1H, dd, J=7.3, 7.3 Hz), 7.21 (2H, d, J=8.4 Hz), 7.36 (2H, dd, J=7.7, 7.3 Hz), 7.51 (1H, d, J=9.9 Hz), 7.58 (2H, d, J=7.7 Hz), 8.24 (1H, brs), 9.77 (1H, S);

MASS (ES+): m/e 620.53 (M+1).

Preparation 316

Compound (316) was obtained in a manner similar to Preparation 313.

¹H-NMR (300 MHz, CDCl₃, δ): 0.82 (3H, t, J=7.3 Hz), 0.89 (3H, t, J=7.0 Hz), 1.20-1.98 (14H, m), 1.27 (3H, s), 2.01-2.39 (4H, m), 2.91 (1H, dd, J=13.5, 6.2 Hz), 3.18 (1H, dd, J=13.5, 9.2 Hz), 3.21-3.37 (3H, m), 3.80-3.91 (1H, m), 4.24 (1H, dt, J=10.3, 7.7 Hz), 4.32 (2H, t, J=6.6 Hz), 4.44 (2H, S), 4.65-4.71 (1H, m), 5.13 (1H, ddd, J=10.6, 9.2, 6.2 Hz), 5.85 (1H, S), 6.55 (1H, br), 6.83 (2H, d, J=8.4 Hz), 7.10 (1H, d, J=10.6 Hz), 7.18 (2H, d, J=8.4 Hz), 7.40-7.48 (2H, m), 7.53-7.60 (1H, m), 7.57 (1H, d, J=10.3 Hz), 8.03 (2H, d, J=8.4 Hz);

MASS (ES+): m/e 720.53 (M+1).

Preparation 317

Compound (317) was obtained in a manner similar to Preparation 77.

¹H-NMR (300 MHz, CDCl₃, δ): 0.83 (3H, t, J=7.3 Hz), 0.89 (3H, t, J=6.6 Hz), 1.22-1.93 (14H, m), 1.28 (3H, S), 2.07-2.41 (4H, m), 2.91 (1H, dd, J=13.5, 6.2 Hz), 3.18 (1H, dd, J=13.5, 9.2 Hz), 3.23-3.38 (3H, m), 3.66 (2H, t, J=6.2 Hz), 3.86 (1H, dt, J=8.8, 4.8 Hz), 4.23 (1H, dt, J=10.3, 7.7 Hz), 4.44 (2H, S), 4.65-4.71 (1H, m), 5.13 (1H, ddd, J=10.3, 9.2, 6.2 Hz), 5.96 (1H, S), 6.55 (1H, br), 6.83 (2H, d, J=8.4 Hz), 7.10 (1H, d, J=10.3 Hz), 7.17 (2H, d, J=8.4 Hz), 7.55 (1H, d, J=10.3 Hz);

MASS (ES+): m/e 616.60 (M+1).

Preparation 318

Compound (318) was obtained in a manner similar to Preparation 78. The obtained compound was used in Example 172.

¹H-NMR (300 MHz, CDCl₃, δ): 0.84 (3H, t, J=7.3 Hz), 0.89 (3H, t, J=7.7 Hz), 1.05-1.40 (4H, m), 1.29 (3H, S), 1.42-1.94 (6H, m), 2.08-2.41 (4H, m), 2.46-2.55 (2H, m), 2.91 (1H, dd, J=13.9, 5.9 Hz), 3.18 (1H, dd, J=13.9, 9.5 Hz), 3.20-3.37 (3H, m), 3.81-3.91 (1H, m), 4.24 (1H, dt, J=10.3, 7.7 Hz), 4.44 (2H, S), 4.64-4.72 (1H, m), 5.13 (1H, ddd, J=9.9, 9.5, 5.9 Hz), 5.94 (1H, s), 6.56 (1H, br), 6.83 (2H, d, J=8.1 Hz), 7.13 (1H, d, J=10.3 Hz), 7.18 (2H, d, J=8.1 Hz), 7.51 (1H, d, J=9.9 Hz), 9.78 (1H, s);

MASS (ES+): m/e 614.61 (M+1).

Preparation 319

Compound (319) was obtained in a manner similar to Example 141 mentioned below.

¹H-NMR (300 MHz, CDCl₃, δ): 0.82 (3H, t, J=7.3 Hz), 1.27 (3H, s), 1.33-1.99 (12H, m), 2.05-2.40 (6H, m), 2.90 (1H, dd, J=13.6, 6.2 Hz), 3.18 (1H, dd, J=13.6, 9.2 Hz), 3.22-3.33 (1H, m), 3.54-3.70 (4H, m), 3.80-3.91 (1H, m), 4.18-4.33 (1H, m), 4.32 (2H, t, J=6.2 Hz), 4.65-4.70 (1H, m), 4.66 (2H, s), 5.13 (1H, dt, J=9.9, 6.2 Hz), 5.81 (1H, 5), 6.84 (2H, d, J=8.8 Hz), 7.08-7.19 (3H, m), 7.40-7.48 (2H, m), 7.50-7.60 (2H, m), 8.00-8.06 (2H, m);

MASS (ES+): m/e 718.38 (M+1).

Preparation 320

Compound (320) was obtained in a manner similar to Preparation 77.

¹H-NMR (300 MHz, CDCl₃, δ): 0.84 (3H, t, J=7.3 Hz), 1.17-1.93 (14H, m), 1.28 (3H, s), 2.07-2.39 (4H, m), 2.88 (1H, dd, J=13.5, 6.2 Hz), 3.18 (1H, dd, J=13.5, 9.9 Hz), 3.20-3.31 (1H, m), 3.42-3.50 (2H, m), 3.51-3.60 (2H, m), 3.66 (2H, t, J=6.6 Hz), 3.79-3.91 (1H, m), 4.23 (1H, dt, J=9.9, 7.7 Hz), 4.64 (2H, s), 4.65-4.71 (1H, m), 5.13 (1H, ddd, J=10.3, 9.9, 6.2 Hz), 5.92 (1H, s), 6.85 (2H, d, J=8.4 Hz), 7.13 (1H, d, J=9.9 Hz), 7.14 (2H, d, J=8.4 Hz), 7.54 (1H, d, J=10.3 Hz);

MASS (ES+): m/e 614.55 (M+1).

Preparation 321

Compound (321) was obtained in a manner similar to Preparation 78. The obtained compound was used in Example 175.

¹H-NMR (300 MHz, CDCl₃, δ): 0.84 (3H, t, J=7.3 Hz), 1.29 (3H, s), 1.46-1.91 (12H, m), 2.03-2.40 (4H, m), 2.45-2.54 (2H, m), 2.88 (1H, dd, J=13.5, 6.2 Hz), 3.18 (1H, dd, J=13.5, 9.5 Hz), 3.20-3.31 (1H, m), 3.42-3.50 (2H, m), 3.51-3.59 (2H, m), 3.79-3.90 (1H, m), 4.23 (1H, dt, J=9.9, 7.0 Hz), 4.64 (2H, s), 4.65-4.70 (1H, m), 5.13 (1H, ddd, J=10.3, 9.5, 6.2 Hz), 5.89 (1H, s), 6.85 (2H, d, J=8.8 Hz), 7.14 (2H, d, J=8.8 Hz), 7.14 (1H, d, J=9.9 Hz), 7.48 (1H, d, J=10.3 Hz), 9.77 (1H, s);

MASS (ES+): m/e 612.60 (M+1).

Preparation 322

To a solution of the Compound (294) (500 mg) in dioxane (6 ml) and water (2 ml) was added a 2M aqueous solution of sodium carbonate (2 ml), and the solution was stirred. To the mixture were added 3-pyridinylboronic acid (170 mg) and dichlorobis(trichlorophosphine)palladium (II) catalyst (48.4 mg). The obtained suspension was degassed with ultrasonic for 1 to 2 min, and the air was purged from the reaction vessel with nitrogen. The suspension was stirred at 95° C. for 1 hour, then cooled to ambient temperature and extracted with ethyl acetate. The extract was washed with water and brine, dried over sodium sulfate and filtered. The filtrate was evaporated and the residue was purified with silica gel column chromatography (eluting with ethyl acetate, then ethyl acetate/methanol=9/1) to give the objected Compound (322).

¹H-NMR (300 MHz, CDCl₃, δ): 0.82 (3H, t, J=7.3 Hz), 1.28 (3H, s), 1.34-1.99 (8H, m), 2.08-2.38 (4H, m), 3.04 (1H, dd, J=13.6, 6.2 Hz), 3.27-3.39 (1H, m), 3.29 (1H, dd, J=13.6, 9.2 Hz), 3.84-3.94 (1H, m), 4.20-4.30 (1H, m), 4.32 (2H, t, J=6.6 Hz), 4.67-4.73 (1H, m), 5.23 (1H, ddd, J=10.3, 9.2, 6.2 Hz), 5.89 (1H, s), 7.11 (1H, d, J=10.6 Hz), 7.32-7.39 (1H, m), 7.35 (2H, d, J=8.4 Hz), 7.40-7.72 (6H, m), 7.83-7.89 (1H, m), 8.03 (2H, d, J=8.4 Hz), 8.56-8.60 (1H, m), 8.81-8.85 (1H, m);

MASS (ES+): m/e 654.50 (M+1).

Preparation 323

Compound (323) was obtained in a manner similar to Preparation 77.

¹H-NMR (300 MHz, CDCl₃, δ): 0.84 (3H, t, J=7.3 Hz), 1.25-1.94 (8H, m), 1.29 (3H, s), 2.11-2.26 (2H, m), 2.26-2.40 (2H, m), 3.04 (1H, dd, J=13.5, 6.2 Hz), 3.29 (1H, dd, J=13.5, 9.2 Hz), 3.30-3.39 (1H, m), 3.62-3.70 (2H, m), 3.84-3.94 (1H, m), 4.24 (1H, dt, J=10.3, 7.7 Hz), 4.67-4.73 (1H, m), 5.23 (1H, ddd, J=10.3, 9.2, 6.2 Hz), 5.97 (1H, s), 7.11 (1H, d, J=10.3 Hz), 7.35 (2H, d, J=8.4 Hz), 7.36 (1H, dd, J=7.7, 0.7 Hz), 7.51 (2H, d, J=8.4 Hz), 7.61 (1H, d, J=10.3 Hz), 7.83-7.88 (1H, m), 8.57 (1H, dd, J=4.8, 1.5 Hz), 8.81-8.84 (1H, m);

MASS (ES+): m/e 550.52 (M+1).

Preparation 324

Compound (324) was obtained in a manner similar to Preparation 78. The obtained compound was used in Example 178.

¹H-NMR (300 MHz, CDCl₃, δ): 0.84 (3H, t, J=7.3 Hz), 1.30 (3H, s), 1.46-1.93 (6H, m), 2.10-2.39 (4H, m), 2.46-2.55 (2H, m), 3.04 (1H, dd, J=13.6, 6.6 Hz), 3.26-3.38 (1H, m), 3.28 (1H, dd, J=13.6, 9.5 Hz), 3.85-3.94 (1H, m), 4.25 (1H, dt, J=10.3, 7.3 Hz), 4.67-4.73 (1H, m), 5.23 (1H, ddd, J=10.3, 9.5, 6.6 Hz), 5.94 (1H, s), 7.13 (1H, d, J=10.3 Hz), 7.35 (2H, d, J=8.4 Hz), 7.36 (1H, d, J=7.7 Hz), 7.51 (2H, d, J=8.4 Hz), 7.56 (1H, d, J=10.3 Hz), 7.83-7.88 (1H, m), 8.58 (1H, dd, J=4.8, 1.8 Hz), 8.81-8.84 (1H, m), 9.78 (1H, s);

MASS (ES+): m/e 548.46 (M+1).

Preparation 325

(2S)-2-amino-3-(3,4-dichlorophenyl)propanoic acid (3.17 g) and sodium bicarbonate (2.28 g) was added to a mixed solvent of dioxane and water (20 ml/20 ml). To the mixture was added Boc₂O (5.91 g) and the mixture was stirred at ambient temperature for 6 hours. To the mixture was added water and the mixture was extracted with ether. The water layer was adjusted to pH 2 with hydrochloric acid and extracted with ethyl acetate. The extract was washed with water and brine, and dried over sodium sulfate. The solvent was removed by evaporation to give the objective Compound (325).

¹H-NMR (300 MHz, CDCl₃, δ): 1.32 (3H, s), 1.43 (6H, s), 2.81-3.08 (1H, m), 3.09-3.26 (1H, m), 4.51-4.64 (1H, m), 4.94-5.05 (1H, m), 7.03 (1H, dd, J=8.4, 2.2 Hz), 7.25-7.34 (1H, m), 7.37 (1H, d, J=8.4 Hz);

MASS (ES−): m/e 332.16 (M−1).

Preparation 326

Compound (326) was obtained in a manner similar to Preparation 13.

¹H-NMR (300 MHz, CDCl₃, δ): 1.34 (2H, s), 1.42 (7H, s), 1.65-1.79 (1H, m), 1.84-2.30 (3H, m), 2.81-3.02 (2.5H, m), 3.54-3.69 (1.5H, m), 4.36-4.47 (1H, m), 4.55-4.67 (1H, m), 5.10 (1H, d, J=12.5 Hz), 5.22 (1H, d, J=12.5 Hz), 5.30 (1H, d, J=8.8 Hz), 7.07 (1H, dd, J=8.1, 1.8 Hz), 7.22-7.41 (6H, m), 7.29 (1H, d, J=1.8 Hz);

MASS (ES+): m/e 521.31 (M+1).

Preparation 327

Compound (327) was obtained in a manner similar to Preparation 14.

¹H-NMR (300 MHz, CDCl₃, δ): 0.81 (3H, t, J=7.3 Hz), 1.39 (3H, s), 1.43 (9H, s), 1.50-2.34 (6H, m), 2.88-3.04 (2.5H, m), 3.46-3.70 (1.5H, m), 4.39-4.46 (1H, m), 4.69-5.06 (2H, m), 5.10 (1H, d, J=12.4 Hz), 5.18 (1H, d, J=12.4 Hz), 6.86 (1H, d, J=8.4 Hz), 7.10 (1H, dd, J=8.4, 2.2 Hz), 7.28-7.40 (7H, m);

MASS (ES+): m/e 620.41 (M+1).

Preparation 328

Compound (328) was obtained in a manner similar to Preparation 15.

¹H-NMR (300 MHz, CDCl₃, δ): 0.75 (3H, t, J=7.3 Hz), 1.34-2.34 (12H, m), 1.44 (9H, s), 1.47 (3H, s), 2.86-3.09 (0.5H, m), 2.91 (1H, dd, J=13.2, 5.9 Hz), 3.02 (1H, dd, J=13.2, 8.4 Hz), 3.51-3.72 (1.5H, m), 3.90-4.10 (1H, m), 4.32 (2H, t, J=6.2 Hz), 4.39-4.46 (1H, m), 4.83-5.06 (2H, m), 5.06-5.23 (2H, m), 6.79-6.95 (1H, m), 6.86 (1H, s), 7.07 (1H, dd, J=8.4, 2.2 Hz), 7.19-7.38 (7H, m), 7.39-7.47 (2H, m), 7.51-7.59 (1H, m), 8.03 (2H, d, J=7.0 Hz);

MASS (ES+): m/e 853.59 (M+1).

Preparation 329

Compound (329) was obtained in a manner similar to Preparation 17.

¹H-NMR (300 MHz, CDCl₃, δ): 0.80 (3H, t, J=7.3 Hz), 1.45 (12H, s), 1.58-2.00 (12H, m), 2.11-2.25 (0.5H, m), 2.81-3.10 (3.5H, m), 3.78-3.92 (1H, m), 4.21-4.43 (3H, m), 4.83-4.93 (1H, m), 5.52-5.63 (1H, m), 6.77 (1H, s), 7.08 (1H, dd, J=8.4, 1.8 Hz), 7.18-7.28 (1H, m), 7.31 (1H, d, J=1.8 Hz), 7.36 (1H, d, J=8.4 Hz), 7.39-7.48 (2H, m), 7.52-7.59 (1H, m), 8.03 (2H, d, J=7.0 Hz);

MASS (ES+): m/e 763.52 (M+1).

Preparation 330

Compound (330) was obtained in a manner similar to Preparation 18.

¹H-NMR (300 MHz, CDCl₃, δ): 0.70 (3H, br), 1.37 (3H, s), 1.50-2.15 (13H, m), 2.75-2.93 (1H, m), 2.94-3.10 (1H, m), 3.11-3.27 (1H, m), 3.65-3.80 (1H, m), 3.97-4.40 (3H, m), 4.83-4.98 (1H, m), 7.00-7.12 (1H, m), 7.27-7.35 (2H, m), 7.35-7.45 (2H, m), 7.49-7.57 (1H, m), 7.62-7.78 (1H, m), 7.99 (2H, d, J=7.3 Hz), 8.03-8.22 (2H, m);

MASS (ES+): m/e 663.45 (M+1).

Preparation 331

Compound (331) was obtained in a manner similar to Preparation 76.

¹H-NM (300 MHz, CDCl₃, δ): 0.81 (3H, t, J=7.3 Hz), 1.28 (3H, s), 1.35-1.56 (2H, m), 1.60-1.98 (6H, m), 2.11-2.38 (4H, m), 2.93 (1H, dd, J=13.9, 6.2 Hz), 3.16 (1H, dd, J=13.9, 9.2 Hz), 3.26-3.37 (1H, m), 3.76-3.89 (1H, m), 4.18-4.49 (1H, m), 4.31 (2H, t, J=6.3 Hz), 4.65-4.73 (1H, m), 5.06-5.17 (1H, m), 6.01 (1H, s), 7.07 (1H, dd, J=8.1, 2.2 Hz), 7.09 (1H, d, J=9.9 Hz), 7.29-7.37 (2H, m), 7.38-7.48 (2H, m), 7.51-7.60 (1H, m), 7.64 (1H, d, J=10.3 Hz), 8.02 (2H, d, J=7.0 Hz);

MASS (ES+): m/e 645.42 (M+1).

Preparation 332

Compound (332) was obtained in a manner similar to Preparation 77.

¹H-NMR (300 MHz, CDCl₃, δ): 0.83 (3H, t, J=7.3 Hz), 1.28 (3H, s), 1.30-1.52 (2H, m), 1.54-2.00 (6H, m), 2.08-2.38 (4H, m), 2.93 (1H, dd, J=13.6, 6.6 Hz), 3.16 (1H, dd, J=13.6, 8.8 Hz), 3.32 (1H, dt, J=9.9, 7, 3 Hz), 3.66 (1H, t, J=6.2 Hz), 3.79-3.89 (1H, m), 4.24 (1H, dt, J=10.3, 7.7 Hz), 4.66-4.72 (1H, m), 5.12 (1H, ddd, J=10.3, 8.8, 6.6 Hz), 6.02 (1H, s), 7.05 (1H, d, J=10.3 Hz), 7.07 (1H, dd, J=8.1, 2.2 Hz), 7.35 (1H, d, J=8.1 Hz), 7.61 (1H, d, J=10.3 Hz);

MASS (ES+): m/e 541.38 (M+1).

Preparation 333

Compound (333) was obtained in a manner similar to Preparation 78. The obtained compound was used in Example 181.

¹H-NMR (300 MHz, CDCl₃, δ): 0.84 (3H, t, J=7.3 Hz), 1.29 (3H, s), 1.48-1.93 (6H, m), 2.10-2.42 (4H, m), 2.46-2.55 (2H, m), 2.93 (1H, dd, J=13.5, 6.6 Hz), 3.16 (1H, dd, J=13.5, 9.2 Hz), 3.27-3.37 (1H, m), 3.81-3.91 (1H, m), 4.25 (1H, dt, J=10.3, 7.7 Hz), 4.66-4.72 (1H, m), 5.12 (1H, ddd, J=9.9, 9.2, 6.6 Hz), 5.89 (1H, s), 7.04 (1H, d, J=9.9 Hz), 7.08 (1H, dd, J=8.1, 2.2 Hz), 7.34 (1H, d, J=2.2 Hz), 7.35 (1H, d, J=8.1 Hz), 7.55 (1H, d, J=10.3 Hz), 9.77 (1H, s);

MASS (ES+): m/e 539.32 (M+1).

Preparation 334

Compound (334) was obtained in a manner similar to Preparation 14.

¹H-NMR (300 MHz, CDCl₃, δ): 1.34-1.64 (4H, m), 1.48 (9H, s), 1.64-1.77 (1H, m), 3.04-3.17 (1H, m), 3.31 (1H, dd, J=12.8, 11.0 Hz), 3.69 (1H, dd, J=12.8, 4.4 Hz), 4.01-4.13 (1H, m), 4.76 (1H, ddd, J=11.0, 8.1, 4.4 Hz), 5.05 (1H, d, J=12.1 Hz), 5.17 (1H, d, J=12.1 Hz), 5.59 (1H, d, J=8.1 Hz), 7.17-7.43 (7H, m), 7.48 (1H, dd, J=8.4, 7.3 Hz), 7.59 (1H, dd, J=8.4, 7.3 Hz), 7.73 (1H, d, J=7.3 Hz), 7.82 (1H, d, J=7.3 Hz), 8.23 (1H, d, J=8.4 HZ);

MASS (ES+): m/e 503.22 (M+1).

Preparation 335

Compound (335) was obtained in a manner similar to Preparation 15.

¹H-NMR (300 MHz, CDCl₃, δ): 0.87 (3H, t, J=7.3 Hz), 1.32-1.63 (4H, m), 1.45 (9H, s), 1.47 (3H, s), 1.63-1.75 (1H, m), 1.86-2.08 (2H, m), 3.02-3.13 (1H, m), 3.22 (1H, dd, J=12.8, 11.0 Hz), 3.77 (1H, dd, J=12.8, 4.0 Hz), 4.10-4.19 (1H, m), 5.00-5.19 (2H, m), 5.06 (1H, d, J=12.5 Hz), 5.13 (1H, d, J=12.5 Hz), 7.08 (1H, d, J=7.7 Hz), 7.20-7.41 (7H, m), 7.43-7.54 (1H, m), 7.56-7.65 (1H, m), 7.74 (1H, dd, J=6.6, 2.6 Hz), 7.82 (1H, d, J=8.4 Hz), 8.36 (1H, d, J=8.4 Hz);

MASS (ES+): m/e 602.51 (M+1).

Preparation 336

Compound (336) was obtained in a manner similar to Preparation 16.

¹H-NMR (300 MHz, CDCl₃, δ): 0.78 (3H, t, J=7.3 Hz), 1.15-2.39 (13H, m), 1.44 (2H, s), 1.46 (7H, s), 1.56 (3H, s), 3.09-3.19 (1H, m), 3.28 (1H, dd, J=12.8, 10.6 Hz), 3.70 (1H, dd, J=12.8, 4.4 Hz), 4.05-4.20 (1H, m), 4.33 (2H, t, J=6.2 Hz), 5.01-5.21 (2H, m), 5.07 (1H, d, J=12.5 Hz), 5.13 (1H, d, J=12.5 Hz), 6.94 (1H, d, J=7.7 Hz), 7.07 (1H, s), 7.25-7.39 (7H, m), 7.38-7.48 (1H, m), 7.48-7.64 (2H, m), 7.71-7.78 (1H, m), 7.83 (1H, d, J=7.7 Hz), 8.03 (2H, d, J=8.4 Hz), 8.29 (1H, d, J=8.4 Hz);

MASS (ES+): m/e 835.45 (M+1).

Preparation 337

Compound (337) was obtained in a manner similar to Preparation 17.

¹H-NMR (300 MHz, CDCl₃, δ): 0.84 (3H, t, J=7.3 Hz), 1.03-2.08 (13H, m), 1.44 (3H, s), 1.45 (6H, s), 1.57 (3H, s), 3.10-3.41 (2H, m), 3.59-3.73 (1H, m), 4.00-4.19 (1H, m), 4.25-4.40 (3H, m), 5.02-5.42 (2H, m), 6.88 (1H, s), 7.26-7.64 (6H, m), 7.72-7.80 (1H, m), 7.84 (1H, d, J=8.1 Hz), 8.04 (2H, d, J=8.4 Hz), 8.24 (1H, d, J=8.8 Hz);

MASS (ES+): m/e 745.41 (M+1).

Preparation 338

Compound (338) was obtained in a manner similar to Preparation 18.

¹H NMR (300 MHz, CDCl₃, δ): 0.72-0.88 (3H, m), 1.10-2.32 (14H, m), 1.43 (3H, s), 3.29-3.63 (2H, m), 3.98-4.08 (1H, m), 4.18-4.43 (3H, m), 5.01-5.18 (1H, m), 7.21-7.59 (6H, m), 7.60-7.75 (1H, m), 7.79 (1H, d, J=8.4 Hz), 7.99 (1H, d, J=7.7 Hz), 8.09-8.65 (4H, m);

MASS (ES+): m/e 645.32 (Free).

Preparation 339

Compound (339) was obtained in a manner similar to Preparation 76.

¹H-NMR (300 MHz, CDCl₃, δ): 0.77 (3H, t, J=7.3 Hz), 1.27 (3H, s), 1.36-2.03 (8H, m), 2.05-2.20 (2H, m), 2.22-2.40 (2H, m), 3.05 (1H, dt, J=9.9, 7.7 Hz), 3.50 (1H, dd, J=14.3, 6.2 Hz), 3.64 (1H, dd, J=14.3, 9.2 Hz), 3.75 (1H, dt, J=9.9, 4.8 Hz), 4.18-4.32 (1H, m), 4.33 (2H, t, J=6.6 Hz), 4.62-4.71 (1H, m), 5.43 (1H, ddd, J=10.3, 9.2, 6.2 Hz), 5.82 (1H, s), 7.15 (1H, d, J=10.3 Hz), 7.35-7.62 (7H, m), 7.66 (1H, d, J=10.3 Hz), 7.70-7.77 (1H, m), 7.85 (1H, d, J=8.1 Hz), 8.04 (2H, d, J=8.4 Hz), 8.13 (1H, d, J=8.8 Hz);

MASS (ES+): m/e 627.44 (M+1).

Preparation 340

Compound (340) was obtained in a manner similar to Preparation 77.

¹H-NMR (300 MHz, CDCl₃, δ): 0.79 (3H, t, J=7.3 Hz), 1.27 (3H, s), 1.30-1.96 (8H, m), 2.07-2.22 (2H, m), 2.23-2.38 (2H, m), 3.05 (1H, dt, J=10.3, 7.7 Hz), 3.50 (1H, dd, J=13.9, 6.6 Hz), 3.57-3.71 (2H, m), 3.64 (1H, dd, J=13.9, 9.2 Hz), 3.75 (1H, dt, J=10.3, 4.4 Hz), 4.24 (1H, dt, J=10.3, 7.7 Hz), 4.61-4.70 (1H, m), 5.43 (1H, ddd, J=10.3, 9.2, 6.6 Hz), 5.95 (1H, s), 7.15 (1H, d, J=10.3 Hz), 7.34-7.41 (2H, m), 7.45-7.53 (1H, m), 7.53-7.61 (1H, m), 7.66 (1H, d, J=10.3 Hz), 7.70-7.77 (1H, m), 7.85 (1H, d, J=8.1 Hz), 8.13 (1H, d, J=8.4 Hz);

MASS (ES+): m/e 523.41 (M+1).

Preparation 341

Compound (341) was obtained in a manner similar to Preparation 78. The obtained compound was used in Example 184.

¹H-NMR (300 MHz, CDCl₃, δ): 0.79 (3H, t, J=7.3 Hz), 1.28 (3H, s), 1.43-1.95 (6H, m), 2.06-2.36 (4H, m), 2.51 (1H, dt, J=6.2, 1.1 Hz), 3.03 (1H, dt, J=9.9, 7.7 Hz), 3.50 (1H, dd, J=14.3, 6.2 Hz), 3.63 (1H, dd, J=14.3, 9.2 Hz), 3.74 (1H, dt, J=9.9, 4.4 Hz), 4.24 (1H, dt, J=10.3, 7.0 Hz), 4.61-4.70 (1H, m), 5.42 (1H, ddd, J=9.9, 9.2, 6.2 Hz), 5.90 (1H, s), 7.16 (1H, d, J=10.3 Hz), 7.33-7.41 (2H, m), 7.45-7.61 (2H, m), 7.60 (1H, d, J=9.9 Hz), 7.70-7.77 (1H, m), 7.85 (1H, d, J=8.1 Hz), 8.12 (1H, d, J=8.4 Hz), 9.78 (1H, t, J=1.1 Hz);

MASS (ES+): m/e 521.33 (M+1).

Preparation 342

The Compound (294) (9.83 g) was dissolved in N,N-dimethylformamide (100 ml), and lithium chloride (4.02 g), tributylvinyltin (5.16 g) and dichlorobis(triphenylphosphine)palladium (II) (476 mg) were added to the mixture under nitrogen atmosphere. The mixture was stirred at 100° C. for 1 day. The reaction mixture was cooled to room temperature, and an aqueous solution of hydrogen fluoride (16 g in water (15 ml)) was added to the mixture and stirred for 60 min. The reaction mixture was diluted with ethyl acetate and the insoluble matter was filtered off. The mixture was partitioned between ethyl acetate and water, and the ethyl acetate layer was washed with an aqueous solution of hydrogen fluoride (10 g in water (100 ml)), water and saturated brine, dried over sodium sulfate and evaporated. The residue was purified by flush chromatography (Silica gel 60N, Spherical, eluting with ethyl acetate/hexane=1/1 then 2/1) to give the objective Compound (342) as a pale yellow foam.

¹H-NMR (300 MHz, CDCl₃, δ): 0.82 (3H, t, J=7.4 Hz), 1.28 (3H, s), 1.36-1.54 (2H, m), 1.67-1.99 (4H, m), 2.08-2.40 (4H, m), 2.95 (1H, dd, J=13.5, 6 Hz), 3.23 (1H, dd, J=13.5, 9.5 Hz), 3.28 (1H, m), 3.86 (1H, m), 4.24 (1H, dt, J=10.3, 7.7 Hz), 4.32 (2H, t, J=6.3 Hz), 4.67 (1H, m), 5.18 (1H, m), 5.21 (1H, d, J=10.8 Hz), 5.71 (1H, d, J=17.6 Hz), 5.87 (1H, s), 6.68 (1H, dd, J=17.6, 10.8 Hz), 7.13 (1H, d, J=10.3 Hz), 7.19 (2×1H, d, J=8.1 Hz), 7.32 (2×1H, d, J=8.1 Hz), 7.44 (2×1H, dd, J=7.5, 7.5 Hz), 7.52-7.60 (2H, m), 8.03 (2×1H, dd, J=7.5, 1.5 Hz);

MASS (ES+): m/e 603.51.

Preparation 343

The Compound (342) was dissolved into a mixed solvent of methanol/dichloromethane (2/1, 60 ml), and the mixture was cooled in dry ice-acetone bath (internal temperature: about 70° C.) and bubbled with 1 to 2% of ozone in oxygen at the velocity of 1 L/min for 15 min. The mixture was stirred under nitrogen atmosphere and then under oxygen atmosphere. To the mixture was added dimethyl sulfide (0.7 ml) and the mixture was stirred with raising the temperature to ambient temperature. The reaction mixture was evaporated and purified by flash column chromatography (Silica gel 60N, Spherical, 110 g, eluting with ethyl acetate/hexane=1/1, 3/2, then 2/1) and preparative thin layer chromatography (eluting with ethyl acetate/hexane=1/1 then methanol/chloroform=1/20) to give the objective Compound (343).

¹H-NMR (300 MHz, CDCl₃, δ): 0.80 (3H, t, J=7.3 Hz), 1.27 (3H, s), 1.36-1.55 (2H, m), 1.58-1.99 (6H, m), 2.07-2.40 (4H, m), 3.08 (1H, dd, J=13.5, 7 Hz), 3.23-3.46 (2H, m), 3.85 (1H, m), 4.25 (1H, m), 4.32 (2H, t, J=6.5 Hz), 4.68 (1H, m), 5.21 (1H, m), 5.89 (1H, s), 7.06 (1H, d, J=10.3 Hz), 7.41 (2×1H, d, J=8.2 Hz), 7.44 (2×1H, dd, J=7.5, 7.5 Hz), 7.56 (1H, dddd, J=7.5, 7.5, 1.5, 1.5 Hz), 7.63 (1H, d, J=10.3 Hz), 7.80 (2×1H, d, J=8.2 Hz), 8.03 (2×1H, dd, J=7.5, 1.5 Hz), 9.97 (1H, s);

MASS (ES+): m/e 605.53.

Preparation 344

To the Compound (343) (6.50 g) were added a solution of 2-methyl-2-butene (4.52 g) in t-butanol (90 ml), a solution of sodium hydrogensulfate (1.93 g) in water (20 ml) and sodium chlorite (4.86 g) in this order. The mixture was stirred at ambient temperature for 2 hours. To the mixture was added a 5% aqueous solution of potassium hydrogensulfide (100 ml) and the mixture was further stirred for 15 min. The mixture was extracted with chloroform (500 ml) and the aqueous layer was further extracted with chloroform (200 ml). The organic layers were combined, washed with saturated brine (200 ml), dried over sodium sulfate and purified by flush chromatography (eluting with ethyl acetate 1/1 then 2/1, ethyl acetate, then 10% methanol in chloroform) to give the objective Compound (344).

¹H-NMR (300 MHz, CDCl₃, δ): 0.81 (3H, t, J=7.3 Hz), 1.28 (3H, m), 1.36-1.57 (3H, m), 1.60-2.41 (9H, m), 3.06 (1H, dd, J=13.8, 6.5 Hz), 3.21-3.38 (2H, m), 3.78-3.93 (1H, m), 4.21-4.37 (3H, m), 4.69 (1H, brd, J=7.0 Hz), 5.14-5.28 (1H, m), 6.05 (1H, s), 7.13 (1H, d, J=10.3 Hz), 7.34 (2H, d, J=8.0 Hz), 7.45 (2H, d, J=8.0 Hz), 7.52-7.60 (1H, m), 7.65 (1H, d, J=10.3 Hz), 7.96-8.08 (4H, m);

MASS (ES−): m/e 619.60(M−1).

Preparation 345

Compound (345) was obtained in a manner similar to Preparation 319.

¹H-NMR (300 MHz, CDCl₃, δ): 0.81 (3H, t, J=7.3 Hz), 1.28 (3H, s), 1.36-2.00 (12H, m), 2.05-2.40 (4H, m), 2.98 (1H, dd, J=13.6, 5.9 Hz), 3.19-3.40 (4H, m), 3.57-3.78 (2H, m), 3.80-3.92 (1H, m), 4.19-4.30 (1H, m), 4.31 (2H, t, J=6.6 Hz), 4.66 (1H, brd, J=5.9 Hz), 5.19 (1H, dt, J=10.3, 6.2 Hz), 5.90 (1H, s), 7.10 (1H, d, J=10.3 Hz), 7.22-7.36 (4H, m), 7.39-7.49 (2H, m), 7.51-7.65 (2H, m), 7.99-8.08 (2H, m);

MASS (ES+): m/e 688.59 (M+1).

Preparation 346

Compound (346) was obtained in a manner similar to Preparation 77.

¹H-NMR (300 MHz, CDCl₃, δ): 0.83 (3H, t, J=7.3 Hz), 1.22-1.74 (9H, m), 1.29 (3H, s), 2.07-2.41 (4H, m), 2.97 (1H, dd, J=13.6, 5.9 Hz), 3.20-3.39 (4H, m), 3.57-3.76 (2H, m), 3.65 (2H, t, J=6.6 Hz), 3.80-3.92 (1H, m), 4.17-4.30 (1H, m), 4.67 (1H, brd, J=5.9 Hz), 5.19 (1H, dt, J=10.3, 5.9 Hz), 6.02 (1H, s), 7.10 (1H, d, J=10.3 Hz), 7.23-7.34 (4H, m), 7.59 (1H, d, J=9.9 Hz);

MASS (ES+): m/e 584.56 (M+I).

Preparation 347

Compound (347) was obtained in a manner similar to Preparation 78. The obtained compound was used in Example 187.

¹H-NMR (300 MHz, CDCl₃, δ): 0.84 (3H, t, J=7.3 Hz), 1.30 (3H, s), 1.41-1.94 (6H, m), 2.08-2.39 (4H, m), 2.50 (2H, brt, J=7.3 Hz), 2.98 (1H, dd, J=13.2, 5.9 Hz), 3.19-3.42 (4H, m), 3.59-3.77 (2H, m), 3.80-3.93 (1H, m), 4.17-4.31 (1H, m), 4.67 (1H, brd, J=5.9 Hz), 5.19 (1H, dt, J=9.9, 6.2 Hz), 5.95 (1H, s), 7.11 (1H, d, J=10.3 Hz), 7.23-7.36 (4H, m), 7.54 (1H, d, J=9.9 Hz), 9.77 (1H, brs);

MASS (ES+): m/e 582.48 (M+1).

Preparation 348

Compound (348) was obtained in a manner similar to Preparation 303.

¹H-NMR (300 MHz, CDCl₃-CD₃OD (9:1 v/v), δ): 0.81 (3H, t, J=7.3 Hz), 1.28 (3H, s), 1.34-1.55 (2H, m), 1.61-1.97 (6H, m), 2.03-2.23 (2H, m), 2.25-2.40 (2H, m), 3.06 (1H, dd, J=13.2, 6.6 Hz), 3.22-3.36 (2H, m), 3.75-3.88 (1H, m), 4.23 (1H, t, J=7.5 Hz), 4.32 (2H, t, J=6.2 Hz), 4.72 (1H, brd, J=6.6 Hz), 5.13-5.25 (1H, m), 7.12-7.20 (1H, t, J=7.3 Hz), 7.33-7.49 (5H, m), 7.53-7.61 (1H, m), 7.62-7.69 (2H, m), 7.76-7.86 (3H, m), 7.99-8.06 (1H, m);

MASS (ES+): m/e 696.44 (M+1).

Preparation 349

Compound (349) was obtained in a manner similar to Preparation 77.

¹H-NMR (300 MHz, CDCl₃, δ): 0.84 (3H, t, J=7.0 Hz), 1.22-1.94 (8H, m), 1.29 (3H, s), 2.05-2.41 (4H, m), 3.06 (1H, dd, J=13.6, 6.6 Hz), 3.20-3.37 (2H, m), 3.66 (2H, brt, J=6.2 Hz), 3.79-3.93 (1H, m), 4.24 (1H, dq, J=10.3, 7.7 Hz), 4.68 (1H, brd, J=5.5 Hz), 5.15-5.28 (1H, m), 6.00 (1H, s), 7.08 (1H, d, J=10.3 Hz), 7.15 (1H, t, J=7.3 Hz), 7.32-7.44 (4H, m), 7.63 (3H, d, J=8.8 Hz), 7.74-7.85 (3H, m);

MASS (ES+): m/e 592.48 (M+1).

Preparation 350

Compound (350) was obtained in a manner similar to Preparation 78. The obtained compound was used in Example 190.

¹H-NMR (300 MHz, CDCl₃, δ): 0.84 (3H, t, J=7.3 Hz), 1.30 (3H, s), 1.45-1.95 (6H, m), 2.07-2.40 (4H, m), 2.51 (2H, brt, J=6.6 Hz), 3.05 (1H, dd, J=13.6, 6.2 Hz), 3.23-3.36 (2H, m), 3.81-3.92 (1H, m), 4.17-4.31 (1H, m), 4.68 (1H, brt, J=5.9 Hz), 5.21 (1H, dt, J=9.6, 6.6 Hz), 5.95 (1H, s), 7.09 (1H, d, J=10.3 Hz), 7.16 (1H, t, J=7.5 Hz), 7.33-7.43 (4H, m), 7.58 (1H, d, J=10.3 Hz), 7.63 (1H, t, J=7.7 Hz), 7.74-7.86 (3H, m), 9.78 (1H, brs);

MASS (ES+): m/e 590.48 (M+1).

Preparation 351

Compound (351) was obtained in a manner similar to Preparation 14.

¹H-NMR (300 MHz, CDCl₃, δ): 0.77 (0.6H, d, J=6.6 Hz), 0.85 (0.6H, d, J=6.6 Hz), 0.92 (2.4H, d, J=6.6 Hz), 0.98 (2.4H, d, J=6.6 Hz), 1.44 (9H, s), 1.86-2.28 (5H, m), 3.50-3.67 (1H, m), 3.82-4.06 (1H, m), 4.35 (1H, dd, J=9.2, 6.2 Hz), 4.49 (1H, dd, J=8.7, 3.6 Hz), 4.96-5.28 (3H, m), 7.30-7.39 (5H, m);

MASS (ES+): m/e 405.30 (M+1).

Preparation 352

Compound (352) was obtained in a manner similar to Preparation 15.

¹H-NMR (300 MHz, CDCl₃, δ): 0.73-0.87 (4H, m), 0.92 (2.5H, d, J=6.6 Hz), 0.97 (2.5H, d, J=6.6 Hz), 1.41 (3H, s), 1.43 (9H, s), 1.78-2.26 (7H, m), 3.50-3.64 (1H, m), 3.84-3.95 (1H, m), 4.49 (1H, dd, J=9.2, 3.3 Hz), 4.67 (1H, dd, J=9.2, 6.6 Hz), 4.92-5.22 (3H, m), 6.58-6.75 (1H, m), 7.28-7.40 (5H, m);

MASS (ES+): m/e 504.37 (M+1).

Preparation 353

Compound (353) was obtained in a manner similar to Preparation 16.

¹H-NMR (300 MHz, CDCl₃, δ): 0.68-0.84 (4H, m), 0.91 (2.5H, d, J=6.6 Hz), 0.96 (2.5H, d, J=6.6 Hz), 1.44 (9H, s), 1.56 (3H, s), 1.66-2.46 (13H, m), 3.51-3.64 (1H, m), 3.81-3.94 (1H, m), 3.98-4.17 (1H, m), 4.32 (2H, brt, J=6.1 Hz), 4.46-4.54 (1H, m), 4.65 (1H, dd, J=9.2, 7.0 Hz), 4.98-5.21 (3H, m), 6.48-6.60 (1H, m), 7.03-7.10 (1H, brs), 7.27-7.65 (8H, m), 8.00-8.07 (2H, m);

MASS (ES+): m/e 737.49 (M+1).

Preparation 354

Compound (354) was obtained in a manner similar to Preparation 17.

¹H-NMR (300 MHz, CDCl₃, δ): 0.83 (3H, t, J=7.3 Hz), 0.95 (6H, d, J=7.0 Hz), 1.44 (9H, s), 1.46 (3H, s), 1.47-3.01 (13H, m), 3.49-3.62 (1H, m), 3.89-4.11 (2H, m), 4.34 (2H, t, J=6.6 Hz), 4.46-4.55 (1H, m), 4.56 (1H, t, J=8.8 Hz), 5.30-5.45 (1H, m), 6.88-6.97 (1H, m), 7.06-7.16 (1H, m), 7.40-7.48 (2H, m), 7.52-7.60 (1H, m), 8.01-8.07 (2H, m);

MASS (ES+): m/e 647.31 (M+1).

Preparation 355

Compound (355) was obtained in a manner similar to Preparation 18.

¹H-NMR (300 MHz, CDCl₃, δ): 0.78-0.98 (9H, m), 1.42 (3H, s), 1.46-2.23 (13H, m), 3.44-3.60 (1H, m), 3.88-4.01 (1H, m), 4.17-4.39 (4H, m), 4.44-4.57 (1H, m), 7.36-7.58 (3H, m), 8.01 (2H, d, J=7.3 Hz), 8.04-8.37 (4H, m);

MASS (ES+): m/e 547.34 (free, M+1).

Preparation 356

Compound (356) was obtained in a manner similar to Preparation 76.

¹H-NMR (300 MHz, CDCl₃, δ): 0.86 (3H, t, J=7.3 Hz), 0.91 (3H, d, J=6.6 Hz), 0.98 (3H, d, J=6.6 Hz), 1.29 (3H, s), 1.36-2.01 (8H, m), 2.11-2.44 (5H, m), 3.47-3.60 (1H, m), 3.83-3.95 (1H, m), 4.19-4.29 (1H, m), 4.31 (2H, t, J=6.6 Hz), 4.48 (1H, t, J=10.3 Hz), 4.75 (1H, brd, J=7.7 Hz), 5.79 (1H, s), 7.16 (1H, d, J=10.6 Hz), 7.38 (1H, d, J=10.6 Hz), 7.40-7.48 (2H, m), 7.52-7.60 (1H, m), 8.00-8.06 (2H, m);

MASS (ES+): m/e 529.48 (M+1).

Preparation 357

Compound (357) was obtained in a manner similar to Preparation 77.

¹H-NMR (300 MHz, CDCl₃, δ): 0.88 (3H, t, J=7.3 Hz), 0.91 (3H, d, J=6.2 Hz), 0.98 (3H, d, J=6.6 Hz), 1.23-1.71 (5H, m), 1.30 (3H, s), 1.76-2.02 (3H, m), 2.12-2.44 (5H, m), 3.47-3.58 (1H, m), 3.60-3.70 (2H, m), 3.83-3.95 (1H, m), 4.23 (1H, dt, J=10.3, 7.7 Hz), 4.48 (1H, t, J=10.3 Hz), 4.75 (1H, brd, J=8.1 Hz), 5.94 (1H, s), 7.17 (1H, d, J=10.3 Hz), 7.39 (1H, d, J=10.6 Hz);

MASS (ES+): m/e 425.36 (M+1).

Preparation 358

Compound (358) was obtained in a manner similar to Preparation 78. The obtained compound was used in Example 193.

¹H-NMR (300 MHz, CDCl₃, δ): 0.88 (3H, t, J=7.3 Hz), 0.90 (3H, d, J=6.6 Hz), 0.99 (3H, d, J=7.0 Hz), 1.31 (3H, s), 1.48-1.75 (5H, m), 1.75-2.02 (3H, m), 2.11-2.45 (5H, m), 2.49 (2H, brt, J=7.3 Hz), 3.53 (1H, dt, J=10.3, 7.3 Hz), 3.84-3.95 (1H, m), 4.17-4.28 (1H, m), 4.47 (1H, t, J=10.3 Hz), 4.75 (1H, dd, J=7.7, 1.8 Hz), 5.85 (1H, s), 7.17 (1H, d, J=10.6 Hz), 7.32 (1H, d, J=10.6 Hz), 9.77 (1H, t, J=1.5 Hz);

MASS (ES+): m/e 423.36 (M+1).

Preparation 359

Compound (359) was obtained in a manner similar to Preparation 13.

¹H-NMR (300 MHz, CDCl₃, δ): 1.42 (9H, s), 2.97-3.26 (2H, m), 3.87 (3H, s), 4.53-4.65 (1H, m), 4.96 (1H, brd, J=7.0 Hz), 6.68-6.77 (2H, m), 7.28 (1H, d, J=8.8 Hz);

MASS (ES−): m/e 328.19(M−1).

Preparation 360

Compound (360) was obtained in a manner similar to Preparation 14.

¹H-NMR (300 MHz, CDCl₃, δ): 1.43 (9H, s), 1.54-1.68 (1H, m), 1.84-2.07 (3H, m), 2.69-2.84 (1H, m), 2.88-3.07 (2H, m), 3.52-3.66 (1H, m), 3.85 (0.5H, s), 3.87 (2.5H, s), 4.36 (1H, dd, J=8.1, 4.4 Hz), 4.58-4.73 (1H, m), 5.03-5.25 (2H, m), 5.34 (1H, d, J=8.4 Hz), 6.69-6.80 (2H, m), 7.17-7.40 (6H, m);

MASS (ES+): m/e 517.29 (M+1).

Preparation 361

Compound (361) was obtained in a manner similar to Preparation 15.

¹H-NMR (300 MHz, CDCl₃, δ): 0.81 (3H, t, J=7.7 Hz), 1.39 (3H, s), 1.43 (9H, s), 1.53-1.72 (1H, m), 1.77-2.05 (5H, m), 2.74-2.88 (1H, m), 2.89-3.08 (2H, m), 3.50-3.65 (1H, m), 3.84 (0.5H, s), 3.88 (2.5H, s), 4.38 (1H, dd, J=8.1, 3.8 Hz), 4.73-5.20 (3H, m), 6.55-6.93 (3H, m), 7.14-7.40 (6H, m);

MASS (ES+): m/e 616.38 (M+1).

Preparation 362

Compound (362) was obtained in a manner similar to Preparation 16.

¹H-NMR (300 MHz, CDCl₃, δ): 0.63 (0.5H, t, J=7.7 Hz), 0.74 (2.5H, t, J=7.7 Hz), 1.30-2.33 (12H, m), 1.41 (9H, s), 1.47 (3H, s), 2.75-3.09 (3H, m), 3.53-3.70 (1H, m), 3.83 (0.5H, s), 3.86 (2.5H, s), 4.00-4.15 (1H, m), 4.26-4.44 (3H, m), 4.88-5.05 (1H, m), 5.07-5.22 (2H, m), 6.55-6.96 (4H, m), 7.14-7.65 (10H, m), 7.99-8.07 (2H, m);

MASS (ES+): m/e 849.51 (M+1).

Preparation 363

Compound (363) was obtained in a manner similar to Preparation 17.

¹H-NMR (300 MHz, CDCl₃, δ): 0.76 (3H, t, J=7.7 Hz), 1.42-2.25 (12H, m), 1.43 (3H, s), 1.44 (9H, s), 2.75-3.45 (3H, m), 3.61-3.81 (1H, m), 3.88 (3H, s), 3.92-4.07 (1H, m), 4.25-4.43 (3H, m), 4.86-5.05 (1H, m), 6.67-6.95 (3H, m), 7.15-7.27 (2H, m), 7.29-7.37 (1H, m), 7.39-7.48 (2H, m), 7.52-7.63 (1H, m), 7.97-8.07 (2H, m);

MASS (ES+): m/e 759.54 (M+1).

Preparation 364

Compound (364) was obtained in a manner similar to Preparation 18.

¹H-NMR (300 MHz, CDCl₃, δ): 0.65-0.80 (3H, m), 1.27-2.37 (12H, m), 1.39 (3H, brs), 2.78-3.20 (3H, m), 3.68-3.93 (3H, m), 3.86 (3H, brs), 4.16-4.43 (3H, m), 4.95 (1H, brs), 6.68-6.77 (1H, m), 6.84 (1H, brs), 7.16-7.24 (1H, m), 7.35-7.45 (2H, m), 7.49-7.58 (1H, m), 7.65-7.75 (1H, m), 7.95-8.03 (2H, m), 8.10-8.30 (3H, m);

MASS (ES+): m/e 659.50 (M+1, free).

Preparation 365

Compound (365) was obtained in a manner similar to Preparation 76.

¹H-NMR (300 MHz, CDCl₃, δ): 0.82 (3H, t, J=7.3 Hz), 1.28 (3H, s), 1.30-2.00 (8H, m), 2.02-2.40 (4H, m), 2.94 (1H, dd, J=13.6, 6.2 Hz), 3.16-3.34 (3H, m), 3.79-3.90 (1H, m), 3.87 (3H, s), 4.19-4.31 (1H, m), 4.32 (2H, t, J=6.6 Hz), 4.64-4.71 (1H, m), 5.17 (1H, dt, J=9.9, 6.2 Hz), 5.88 (1H, brs), 6.77 (1H, dd, J=7.7, 1.5 Hz), 6.80-6.84 (1H, m), 7.09 (1H, d, J=10.3 Hz), 7.25 (1H, d, J=7.3 Hz), 7.39-7.48 (2H, m), 7.52-7.63 (2H, m), 8.00-8.06 (2H, m);

MASS (ES+): m/e 641.50 (M+1).

Preparation 366

Compound (366) was obtained in a manner similar to Preparation 77.

¹H-NMR (300 MHz, CDCl₃, δ): 0.84 (3H, t, J=7.3 Hz), 1.21-1.94 (8H, m), 1.29 (3H, s), 2.07-2.40 (4H, m), 2.94 (1H, dd, J=13.6, 6.2 Hz), 3.21 (1H, dd, J=13.6, 9.5 Hz), 3.23-3.24 (1H, m), 3.60-3.70 (2H, m), 3.80-3.90 (1H, m), 3.87 (3H, s), 4.18-4.30 (1H, m), 4.68 (1H, brd, J=5.9 Hz), 5.17 (1H, dt, J=10.3, 6.2 Hz), 6.01 (1H, brs), 6.77 (1H, dd, J=8.1, 1.8 Hz), 6.82 (1H, brs), 7.09 (1H, d, J=9.9 Hz), 7.25 (1H, d, J=7.7 Hz), 7.60 (1H, d, J=9.9 Hz);

MASS (ES+): m/e 537.46 (M+1).

Preparation 367

Compound (367) was obtained in a manner similar to Preparation 78. The obtained compound was used in Example 196.

¹H-NMR (300 MHz, CDCl₃, δ): 0.84 (3H, t, J=7.7 Hz), 1.29 (3H, s), 1.47-1.95 (6H, m), 2.07-2.41 (4H, m), 2.50 (2H, brt, J=7.0 Hz), 2.94 (1H, dd, J=13.6, 6.2 Hz), 3.20 (1H, dd, J=13.6, 9.5 Hz), 3.21-3.34 (1H, m), 3.79-3.92 (1H, m), 3.87 (3H, s), 4.18-4.30 (1H, m), 4.68 (1H, brd, J=5.9 Hz), 5.16 (1H, dt, J=9.9, 6.2 Hz), 5.92 (1H, brs), 6.77 (1H, dd, J=8.1, 1.8 Hz), 6.81 (1H, brs), 7.10 (1H, d, J=10.3 Hz), 7.25 (1H, d, J=8.1 Hz), 7.54 (1H, d, J=10.3 Hz), 9.77 (1H, s);

MASS (ES+): m/e 535.43 (M+1).

Preparation 368

Compound (368) was obtained in a manner similar to Preparation 14.

¹H-NMR (300 MHz, CDCl₃, δ): 1.33 (2H, s), 1.42 (7H, s), 1.53-1.69 (1H, m), 1.78-2.06 (3H, m), 2.68-2.84 (1H, m), 2.89-3.08 (2H, m), 3.52-3.66 (1H, m), 4.39 (1H, dd, J=7.7, 4.0 Hz), 4.58-4.69 (1H, m), 5.10 (1H, d, J=12.5 Hz), 5.20 (1H, d, J=12.5 Hz), 5.35 (1H, brd, J=8.8 Hz), 6.78-6.96 (3H, m), 7.00 (1H, brd, J=7.7 Hz), 7.13-7.28 (2H, m), 7.28-7.42 (4H, m);

MASS (ES+): m/e 471.25 (M+1).

Preparation 369

Compound (369) was obtained in a manner similar to Preparation 15.

¹H-NMR (300 MHz, CDCl₃, δ): 0.80 (3H, t, J=7.7 Hz), 1.38 (3H, s), 1.39 (2H, s), 1.49 (7H, s), 1.54-1.70 (1H, m), 1.76-2.08 (5H, m), 2.72-2.90 (1H, m), 2.97 (1H, dd, J=13.2, 8.8 Hz), 3.05 (1H, dd, J=13.2, 5.1 Hz), 3.47-3.67 (1H, m), 4.40 (1H, dd, J=8.4, 4.0 Hz), 4.94 (1H, dt, J=8.8, 5.4 Hz), 4.99-5.25 (3H, m), 6.64-7.04 (4H, m), 7.11-7.26 (2H, m), 7.27-7.40 (4H, m);

MASS (ES+): m/e 570.44 (M+1).

Preparation 370

Compound (370) was obtained in a manner similar to Preparation 16.

¹H-NM (300 MHz, CDCl₃, δ): 0.60 (0.6H, t, J=7.2 Hz), 0.73 (2.4H, t, J=7.2 Hz), 1.35-2.34 (12H, m), 1.44 (3H, s), 1.48 (9H, s), 2.76-3.12 (3H, m), 3.51-3.70 (1H, m), 3.90-4.16 (1H, m), 4.31 (2H, t, J=7.0 Hz), 4.42 (1H, dd, J=8.2, 4.0 Hz), 4.98-5.02 (1H, m), 5.04-5.25 (2H, m), 6.69-7.03 (5H, m), 7.08-7.66 (10H, m), 7.99-8.08 (2H, m);

MASS (ES+): m/e 803.40 (M+1).

Preparation 371

Compound (371) was obtained in a manner similar to Preparation 17.

¹H-NMR (300 MHz, CDCl₃, δ): 0.77 (3H, t, J=7.3 Hz), 1.37-2.25 (12H, m), 1.43 (3H, s), 1.45 (9H, s), 2.82-3.16 (3H, m), 3.65-3.80 (1H, m), 3.93-4.08 (1H, m), 4.25-4.45 (3H, m), 4.89-5.02 (1H, m), 5.30 (1H, brs), 6.82 (1H, brs), 6.88-7.06 (3H, m), 7.20-7.30 (1H, m), 7.33 (1H, brd, J=10.3 Hz), 7.39-7.49 (2H, m), 7.52-7.62 (1H, m), 7.99-8.08 (2H, m);

MASS (ES+): m/e 711.23(M−1).

Preparation 372

Compound (372) was obtained in a manner similar to Preparation 76.

¹H-NMR (300 MHz, CDCl₃, δ): 0.81 (3H, t, J=7.3 Hz), 1.28 (3H, s), 1.33-1.57 (2H, m), 1.61-1.96 (6H, m), 2.07-2.37 (4H, m), 2.98 (1H, dd, J=13.6, 6.2 Hz), 3.06-3.35 (2H, m), 3.81 (1H, dt, J=9.2, 4.4 Hz), 4.18-4.28 (1H, m), 4.32 (2H, t, J=6.2 Hz), 4.73 (1H, brd, J=7.7 Hz), 5.09-5.20 (1H, m), 6.85-7.05 (4H, m), 7.20-7.35 (2H, m), 7.41-7.49 (2H, m), 7.54-7.61 (1H, m), 7.76 (1H, d, J=10.3 Hz), 7.99-8.06 (2H, m).

Preparation 373

Compound (373) was obtained in a manner similar to Preparation 77.

¹H-NMR (300 MHz, CDCl₃, δ): 0.84 (3H, t, J=7.3 Hz), 1.23-1.71 (6H, m), 1.29 (3H, s), 1.73-1.96 (2H, m), 2.08-2.42 (4H, m), 2.97 (1H, dd, J=13.6, 6.2 Hz), 3.23 (1H, dd, J=13.6, 9.5 Hz), 3.27-3.36 (1H, m), 3.61-3.71 (2H, m), 3.81-3.92 (1H, m), 4.24 (1H, dt, J=10.3, 7.3 Hz), 4.70 (1H, brd, J=5.5 Hz), 5.17 (1H, dt, J=9.5, 6.6 Hz), 5.98 (1H, s), 6.87-6.99 (2H, m), 7.02 (1H, d, J=7.7 Hz), 7.10 (1H, d, J=10.3 Hz), 7.20-7.32 (1H, m), 7.59 (1H, d, J=10.3 Hz);

MASS (ES+): m/e 491.32 (M+1).

Preparation 374

Compound (374) was obtained in a manner similar to Preparation 78. The obtained compound was used in Example 199.

¹H-NMR (300 MHz, CDCl₃, δ): 0.83 (3H, t, J=7.7 Hz), 1.29 (3H, s), 1.50-1.93 (4H, m), 2.09-2.40 (6H, m), 2.51 (2H, brt, J=6.3 Hz), 2.97 (1H, dd, J=13.6, 6.6 Hz), 3.22 (1H, dd, J=13.6, 9.2 Hz), 3.24-3.36 (1H, m), 3.81-3.92 (1H, m), 4.24 (1H, dt, J=10.3, 7.3 Hz), 4.65-4.72 (1H, m), 5.16 (1H, dt, J=10.3, 6.6 Hz), 5.88 (1H, brs), 6.86-6.98 (2H, m), 7.01 (1H, d, J=10.3 Hz), 7.10 (1H, d, J=10.3 Hz), 7.19-7.29 (1H, m), 7.52 (1H, d, J=10.3 Hz), 9.77 (1H, t, J=1.5 Hz);

MASS (ES+): m/e 489.23 (M+1).

Preparation 375

To a stirred solution of ethyl R-mandelate (7.0 g) in N,N-dimethylformamide (70 mL) was added imidazole (2.91 g) followed by tert-butyldiphenylchlorosilane (10.7 g) at ambient temperature and the resulting mixture was stirred at the same temperature for two hours. To this mixture was added additional tert-butyldiphenylchlorosilane (1.07 g) and imidazole (530 mg) and the mixture was stirred at ambient temperature for sixteen hours. The reaction mixture was poured into water and extracted with ethyl acetate. The organic layer was successively washed with water, 0.2 N hydrochloric acid, saturated aqueous sodium bicarbonate solution and brine. The organic layer was dried over magnesium sulfate, filtered and evaporated to give Compound (375) (17.4 g) as a pale yellow oil, which was used in the next step without further purification.

¹H-NMR (300 MHz, CDCl₃, δ): 1.05 (3H, t, J=7.3 Hz), 1.11 (9H, s), 3.85-3.92 (2H, m), 5.13 (1H, s), 7.14-7.54 (11H, m), 7.69-7.76 (4H, m).

Preparation 376

Compound (376) was obtained in a manner similar to Preparation 117.

¹H-NMR (300 MHz, CDCl₃, δ): 1.11 (9H, s), 2.92 (1H, dd, J=21.6, 15.8 Hz), 3.18 (1H, dd, J=20.1, 15.8 Hz), 3.49 (3H, d, J=11.4 Hz), 3.59 (3H, d, J=11.4 Hz), 5.21 (1H, s), 7.21-7.49 (13H, m), 7.62-7.68 (2H, m);

MASS (ES+): m/e 519.10(M+Na).

Preparation 377

Compound (377) was obtained in a manner similar to Preparation 14.

¹H-NMR (300 MHz, CDCl₃, δ): 1.32 (9×⅙H, s), 1.41 (9×⅚H, s), 1.68 (1H, m), 1.84-2.32 (3H, m), 2.73 (1×⅙H, dd, J=14, 10 Hz), 2.91-3.06 (3+⅚H, m), 3.64 (1H, m), 4.39 (1H, dd, J=8, 4 Hz), 4.69 (1H, brdt, J=8, 7 Hz), 5.11 (1H, d, J=12 Hz), 5.21 (1H, d, J=12 Hz), 5.31 (1H, d, J=8 Hz), 6.97 (2×⅙H, d, J=6 Hz), 7.16 (2×⅚H, d, J=6 Hz), 7.29-7.41 (5H, m), 8.43 (2×⅙H, d, J=6 Hz), 8.50 (2×⅚H, d, J=6 Hz);

MASS (ES+): m/e 454.41.

Preparation 378

Compound (378) was obtained in a manner similar to Preparation 15.

¹H-NMR (300 MHz, DMSO-d₆, δ): 1.84-1.98 (3H, m), 2.18 (1H, m), 3.30 (1H, dd, J=14, 7.5 Hz), 3.40-3.58 (2H, m), 3.84 (1H, m), 4.41 (1H, dd, J=8.5, 2.5 Hz), 4.72 (1H, br), 5.09 (1H, d, J=12.5 Hz), 5.19 (1H, d, J=12.5 Hz), 7.30-7.44 (5H, m), 7.78 (2×⅙H, d, J=6.5 Hz), 7.91 (2×⅚H, d, J=6.5 Hz), 8.56 (2×⅚H, br), 8.64 (2×⅙H, br), 8.84 (2H, d, J=6.5 Hz);

MASS (ES+): m/e 354.25.

Preparation 379

Compound (379) was obtained in a manner similar to Preparation 15.

¹H-NMR (300 MHz, CDCl₃, δ): 0.57 (3×¼H, t, J=7.5 Hz), 0.80 (3×¾H, t, J=7.5 Hz), 1.31 (3/1/4H, s), 1.37 (9×¼H, s), 1.39 (3×¾H, s), 1.43 (9×¾H, s), 1.65-2.35 (6H, m), 2.86 (1×¼H, dd, J=14, 9.5 Hz), 2.96-3.24 (3+¾H, m), 3.72 (1H, m), 4.41 (1×¾H, dd, J=8, 3.5 Hz), 4.75-5.22 (4+¼H, m), 6.74 (1×¼H, d, J=8.5 Hz), 6.91 (1×¾H, d, J=8.5 Hz), 7.01 (2×¼H, d, J=6 Hz), 7.22 (2×¾H, d, J=6 Hz), 7.29-7.42 (5H, m), 8.40 (2×¼H, d, J=6 Hz), 8.50 (2×¾H, d, J=6 Hz);

MASS (ES+): m/e 553.30.

Preparation 380

Compound (380) was obtained in a manner similar to Preparation 16.

¹H-NMR (300 MHz, DMSO-d₆, δ): 0.72 (3×⅙H, t, J=7.3 Hz), 0.76 (3×⅚H, t, J=7.3 Hz), 1.27 (3H, s), 1.62-2.31 (6H, m), 3.05-3.82 (4H, m), 4.41 (1H, dd, J=8, 4 Hz), 5.03 (1H, d, J=12, 5 Hz), 5.07 (1H, m), 5.18 (1H, d, J=12.5 Hz), 7.30-7.42 (5H, m), 7.57 (2×⅙H, d, J=6 Hz), 7.87 (2×⅚H, d, J=6 Hz), 8.04 (2×⅚H, s), 8.13 (2×⅙H, s), 8.69-9.00 (3H,

MASS (ES+): m/e 453.24.

Preparation 381

Compound (381) was obtained in a manner similar to Preparation 16.

¹H-NMR (300 MHz, CDCl₃, δ): 0.63 (1×¾H, t, J=7 Hz), 0.75 (3×¾H, t, J=7 Hz), 1.32-2.32 (24H, m), 2.90-3.23 (3H, m), 3.53-4.47 (5H, m), 4.92-5.21 (4H, m), 6.82-6.98 (2H, m), 7.04 (2×¼H, d, J=6 Hz), 7.22 (2×¾H, d, J=6 Hz), 7.24-7.38 (5H, m), 7.43 (2×1H, dd, J=7.5, 7.5 Hz), 7.55 (1H, dd, J=7.5, 7.5 Hz), 8.03 (2×1H, d, J=7.5 Hz), 8.41 (2×¼H, d, J=6 Hz), 8.50 (2×¾H, d, J=6 Hz);

MASS (ES+): m/e 786.26.

Preparation 382

Compound (382) was obtained in a manner similar to Preparation 17.

¹H-NMR (300 MHz, CDCl₃, δ): 0.62-0.86 (3H, m), 1.26-2.32 (24H, m), 3.04-3.85 (4H, m), 3.90-5.58 (7H, m), 7.37-7.49 (2H, m), 7.55 (1H, m), 7.85-8.10 (4H, m), 8.67 (2H, br);

MASS (ES+): m/e 696.29.

Preparation 383

Compound (383) was obtained in a manner similar to Preparation 18.

¹H-NMR (300 MHz, CDCl₃, δ): 0.67 (3H, m), 1.22-2.28 (15H, m), 3.04-3.76 (4H, m), 4.21-4.44 (2H, m), 4.60-5.40 (3H, m), 7.41 (2×1H, brdd, J=7, 7 Hz), 7.55 (1H, brdd, J=7, 7 Hz), 7.66-8.04 (6H, m), 8.67 (2H, br);

MASS (ES−): m/e 594.39.

Preparation 384

Compound (384) was obtained in a manner similar to Preparation 76.

¹H-NMR (300 MHz, CDCl₃, δ): 0.80 (3H, t, J=7.4 Hz), 1.28 (3H, s), 1.38-1.58 (2H, m), 1.62-1.98 (6H, m), 2.07-2.40 (4H, m), 3.02 (1H, m), 3.22 (1H, dd, J=14, 9 Hz), 3.36 (1H, m), 3.86 (1H, m), 4.26 (1H, m), 4.32 (2H, t, J=6.5 Hz), 4.69 (1H, m), 5.21 (1H, m), 5.84 (1H, s), 7.02 (1H, d, J=10 Hz), 7.19 (2×1H, d, J=5.5 Hz), 7.44 (2×1H, dd, J=7.5, 7.5 Hz), 7.56 (1H, m), 7.63 (1H, d, J=10 Hz), 8.03 (2×1H, brd, J=7.5 Hz), 8.52 (2×1H, d, J=5.5 Hz);

MASS (ES+): m/e 578.31.

Preparation 385

Compound (385) was obtained in a manner similar to Preparation 77.

¹H-NMR (300 MHz, CDCl₃, δ): 0.82 (3H, t, J=7.3 Hz), 1.20-1.94 (8H, m), 1.28 (3H, s), 2.04-2.42 (4H, m), 3.01 (1H, dd, J=14, 7 Hz), 3.21 (1H, dd, J=14, 8.7 Hz), 3.34 (1H, m), 3.66 (2H, t, J=6.2 Hz), 3.86 (1H, m), 4.24 (1H, dt, J=10.2, 7.7 Hz), 4.69 (1H, m), 5.21 (1H, ddd, J=10.2, 8.7, 7 Hz), 6.00 (1H, s), 7.03 (1H, d, J=10.2 Hz), 7.18 (2×1H, d, J=4.5 Hz), 7.64 (1H, d, J=10.2 Hz), 8.51 (2×1H, d, J=4.5 Hz);

MASS (ES+): m/e 474.36.

Preparation 386

Compound (386) was obtained in a manner similar to Preparation 78. The obtained compound was used in Example 208.

¹H-NMR (300 MHz, CDCl₃, δ): 0.82 (3H, t, J=7.3 Hz), 1.29 (3H, s), 1.50-1.94 (6H, m), 2.08-2.40 (4H, m), 2.51 (2H, m), 3.01 (1H, dd, J=14, 7 Hz), 3.21 (1H, dd, J=14, 8.5 Hz), 3.34 (1H, m), 3.86 (1H, m), 4.24 (1H, m), 4.69 (1H, m), 5.21 (1H, ddd, J=10, 8.5, 7 Hz), 5.88 (1H, s), 7.03 (1H, d, J=10 Hz), 7.17 (2×1H, d, J=6 Hz), 7.58 (1H, d, J=10 Hz), 8.51 (2×1H, d, J=6 Hz), 9.77 (1H, s);

MASS (ES+): m/e 472.35.

Preparation 387

Compound (387) was obtained in a manner similar to Preparation 311.

¹H-NMR (300 MHz, CDCl₃, δ): 0.82 (3H, t, J=7.3 Hz), 1.28 (3H, s), 1.36-1.98 (8H, m), 2.06-2.40 (4H, m), 2.89 (1H, dd, J=13.5, 6 Hz), 3.18 (1H, dd, J=13.5, 10 Hz), 3.26 (1H, m), 3.86 (1H, m), 4.24 (1H, dt, J=10, 7.5 Hz), 4.32 (2H, t, J=6.5 Hz), 4.50 (2H, brd, J=5 Hz), 4.67 (1H, dd, J=8, 2 Hz), 5.13 (1H, ddd, J=10, 10, 6 Hz), 5.28 (1H, brd, J=10.5 Hz), 5.40 (1H, brd, J=17 Hz), 5.79 (1H, s), 6.04 (1H, ddt, J=17, 10.5, 5 Hz), 6.83 (2×1H, d, J=8.5 Hz), 7.09-7.20 (3H, m), 7.44 (2×1H, dd, J=8, 8 Hz), 7.52 (1H, d, J=10 Hz), 7.54 (1H, m), 8.03 (2×1H, brd, J=8 Hz);

MASS (ES+): m/e 633.32.

Preparation 388

Compound (388) was obtained in a manner similar to Preparation 77.

¹H-NMR (300 MHz, CDCl₃, δ): 0.84 (3H, t, J=7.3 Hz), 1.22-1.48 (3H, m), 1.57-1.92 (5H, m), 2.09-2.42 (4H, m), 2.89 (1H, dd, J=14, 6 Hz), 3.18 (1H, dd, J=14, 10 Hz), 3.26 (1H, m), 3.86 (1H, m), 4.05 (2H, t, J=6.5 Hz), 4.21 (1H, m), 4.50 (2H, m), 4.67 (1H, dd, J=8, 2 Hz), 5.13 (1H, ddd, J=10.5, 10, 6 Hz), 5.28 (1H, dd, J=10, 1.5 Hz), 5.40 (1H, dd, J=17, 1.5 Hz), 5.81 (1H, s), 6.04 (1H, m), 6.82 (2×1H, d, J=8.5 Hz), 7.13 (1H, d, J=10 Hz), 7.14 (2×1H, d, J=8.5 Hz), 7.52 (1H, d, J=10.5 Hz);

MASS (ES+): m/e 571.36.

Preparation 389

Compound (389) was obtained in a manner similar to Preparation 77.

¹H-NMR (300 MHz, CDCl₃, δ): 0.84 (3H, t, J=7.3 Hz), 1.22-1.94 (9H, m), 1.28 (3H, s), 2.08-2.41 (4H, m), 2.87 (1H, dd, J=13.5, 6 Hz), 3.18 (1H, dd, J=13.5, 10 Hz), 3.26 (1H, m), 3.65 (2H, br), 3.85 (1H, m), 4.23 (1H, m), 4.50 (2H, m), 4.67 (1H, m), 5.13 (1H, ddd, J=10, 10, 6 Hz), 5.28 (1H, dd, J=10.5, 2 Hz), 5.41 (1H, dd, J=17.5, 2 Hz), 5.89 (1H, s), 6.04 (1H, m), 6.82 (2×1H, d, J=8.5 Hz), 7.14 (2×1H, d, J=8.5 Hz), 7.14 (1H, d, J=10 Hz), 7.52 (1H, d, J=10 Hz);

MASS (ES+): m/e 529.37.

Preparation 390

Compound (390) was obtained in a manner similar to Preparation 78. The obtained compound was used in Example 211.

¹H-NMR (300 MHz, CDCl₃, δ): 0.84 (3H, t, J=7.3 Hz), 1.29 (3×3H, s), 1.48-1.92 (6H, m), 2.08-2.40 (4H, m), 2.50 (2H, m), 2.88 (1H, dd, J=13.5, 6 Hz), 3.17 (1H, dd, J=13.5, 10 Hz), 3.24 (1H, m), 3.86 (1H, m), 4.22 (1H, m), 4.49 (2H, ddd, J=5, 1.5, 1.5 Hz), 4.66 (1H, m), 5.13 (1H, ddd, J=10, 10, 6 Hz), 5.17 (1H, ddt, J=10.5, 1.5, 1.5 Hz), 5.40 (1H, ddt, J=17, 1.5, 1.5 Hz), 5.83 (1H, s), 6.04 (1H, ddt, J=17, 10.5, 5 Hz), 6.82 (2×1H, d, J=8.5 Hz), 7.13 (2×1H, d, J=8.5 Hz), 7.14 (1H, d, J=10 Hz), 7.46 (1H, d, J=10 Hz), 9.77 (1H, s);

MASS (ES+): m/e 527.40.

Preparation 391

Compound (391) was obtained in a manner similar to Preparation 311.

¹H-NMR (300 MHz, CDCl₃, δ): 0.82 (3H, t, J=7.3 Hz), 1.27 (3H, s), 1.31 (2×3H, d, J=6 Hz), 1.38-1.55 (2H, m), 1.58-2.00 (6H, m), 2.06-2.40 (4H, m), 2.88 (1H, dd, J=13.5, 6 Hz), 3.17 (1H, dd, J=13.5, 10 Hz), 3.26 (1H, m), 3.86 (1H, m), 4.24 (1H, dt, J=10, 7.7 Hz), 4.31 (2H, t, J=6.5 Hz), 4.49 (1H, qq, J=6, 6 Hz), 4.67 (1H, dd, J=8, 2 Hz), 5.14 (1H, ddd, J=10, 10, 6 Hz), 5.80 (1H, s), 6.79 (2×1H, d, J=8.8 Hz), 7.12 (2×1H, d, J=8.8 Hz), 7.14 (1H, d, J=10 Hz), 7.44 (2×1H, dd, J=7.5, 7.5 Hz), 7.52 (1H, d, J=10 Hz), 7.56 (1H, m), 8.03 (2×1H, dd, J=7.5, 1.5 Hz);

MASS (ES+): m/e 635.29.

Preparation 392

Compound (392) was obtained in a manner similar to Preparation 77.

¹H-NMR (300 MHz, CDCl₃, δ): 0.84 (3H, t, J=7.3 Hz), 1.28 (3H, S), 1.31 (2×3H, d, J=6.3 Hz), 1.34-1.94 (8H, m), 2.08-2.39 (4H, m), 2.88 (1H, dd, J=13.5, 6 Hz), 3.17 (1H, dd, J=13.5, 9.5 Hz), 3.26 (1H, m), 3.66 (2H, t, J=6.2 Hz), 3.86 (1H, m), 4.22 (1H, dt, J=10, 7.5 Hz), 4.49 (1H, qq, J=6.3, 6.3 Hz), 4.67 (1H, dd, J=8, 2 Hz), 5.14 (1H, ddd, J=10, 9.5, 6 Hz), 5.89 (1H, S), 6.79 (2×1H, d, J=8.5 Hz), 7.12 (2×1H, d, J=8.5 Hz), 7.14 (1H, d, J=10 Hz), 7.51 (1H, d, J=10 Hz);

MASS (ES+): m/e 531.46.

Preparation 393

Compound (393) was obtained in a manner similar to Preparation 78. The obtained compound was used in Example 214.

¹H-NMR (300 MHz, CDCl₃, δ): 0.84 (3H, t, J=7.4 Hz), 1.29 (3H, s), 1.31 (3H, d, J=6.5 Hz), 1.49-1.91 (6H, m), 2.08-2.40 (4H, m), 2.50 (2H, m), 2.88 (1H, dd, J=13.5, 6 Hz), 3.17 (1H, dd, J=13.5, 10 Hz), 3.26 (1H, m), 3.86 (1H, m), 4.22 (1H, dt, J=10, 7.5 Hz), 4.49 (1H, qq, J=6.5, 6.5 Hz), 4.67 (1H, dd, J=8, 2.5 Hz), 5.13 (1H, ddd, J=10, 10, 6 Hz), 5.83 (1H, s), 6.79 (2×1H, d, J=9 Hz), 7.12 (2×1H, d, J=9 Hz), 7.15 (1H, d, J=10 Hz), 7.44 (1H, d, J=10 Hz), 9.77 (1H, t, J=1.5 Hz);

MASS (ES+): m/e 529.38.

Preparation 394

Compound (394) was obtained in a manner similar to Preparation 311.

¹H-NMR (300 MHz, CDCl₃, δ): 0.82 (3H, t, J=7.3 Hz), 1.27 (3H, s), 1.38-1.98 (11H, m), 2.08-2.40 (4H, m), 2.88 (1H, dd, J=13.5, 6 Hz), 3.18 (1H, dd, J=13.5, 10 Hz), 3.26 (1H, m), 3.85 (1H, m), 4.24 (1H, dt, J=10, 7.5 Hz), 4.31 (2H, t, J=6.5 Hz), 4.41 (2×⅘H, brd, J=6 Hz), 4.55 (2×⅕H, brd, J=6 Hz), 4.67 (1H, m), 5.13 (1H, ddd, J=10, 10, 6 Hz), 5.65-5.91 (2H, m), 5.80 (1H, s), 6.81 (2×1H, d, J=8.7 Hz), 7.13 (2×1H, d, J=8.7 Hz), 7.14 (1H, d, J=10 Hz), 7.44 (2×1H, dd, J=7.5, 7.5 Hz), 7.52 (1H, d, J=10 Hz), 7.56 (1H, m), 8.03 (2×1H, dd, J=7.5, 1.5 Hz);

MASS (ES+): m/e 647.39.

Preparation 395

Compound (395) was obtained in a manner similar to Example 3 mentioned below.

¹H-NMR (300 MHz, CDCl₃, δ): 0.83 (3H, t, J=7.3 Hz), 0.96 (3H, t, J=7.3 Hz), 1.27 (3H, s), 1.36-1.55 (4H, m), 1.57-1.98 (8H, m), 2.06-2.40 (4H, m), 2.88 (1H, dd, J=13.5, 6 Hz), 3.18 (1H, dd, J=13.5, 10 Hz), 3.26 (1H, m), 3.86 (1H, m), 3.92 (2H, t, J=6.5 Hz), 4.24 (1H, dt, J=10, 7.5 Hz), 4.31 (2H, t, J=6.5 Hz), 4.66 (1H, dd, J=8, 2 Hz), 5.13 (1H, ddd, J=10, 10, 6 Hz), 5.79 (1H, s), 6.80 (2×1H, d, J=8.8 Hz), 7.12 (2×1H, d, J=8.8 Hz), 7.14 (1H, d, J=10 Hz), 7.44 (2×1H, dd, J=7.5, 7.5 Hz), 7.52 (1H, d, J=10 Hz), 7.56 (1H, m), 8.03 (1H, dd, J=7.5, 1.5 Hz);

MASS (ES+): m/e 649.41.

Preparation 396

Compound (396) was obtained in a manner similar to Preparation 77.

¹H-NMR (300 MHz, CDCl₃, δ): 0.84 (3H, t, J=7.3 Hz), 0.96 (3H, t, J=7.3, Hz), 1.24-1.93 (12H, m), 1.28 (3H, s), 2.08-2.40 (4H, m), 2.88 (1H, dd, J=13.5, 5.5 Hz), 3.17 (1H, dd, J=13.5, 10 Hz), 3.26 (1H, m), 3.65 (2H, brt, J=6.2 Hz), 3.86 (1H, m), 3.91 (2H, t, J=6.5 Hz), 4.22 (1H, dt, J=10, 7.5 Hz), 4.67 (1H, dd, J=8, 2.5 Hz), 5.13 (1H, ddd, J=10, 10, 5.5 Hz), 5.88 (1H, s), 6.80 (2×1H, d, J=8.5 Hz), 7.12 (2×1H, d, J=8.5 Hz), 7.13 (1H, d, J=10 Hz), 7.51 (1H, d, J=10 Hz);

MASS (ES+): m/e 545.35.

Preparation 397

Compound (397) was obtained in a manner similar to Preparation 78. The obtained compound was used in Example 217.

¹H-NMR (300 MHz, CDCl₃, δ): 0.85 (3H, t, J=7.3 Hz), 0.96 (3H, t, J=7.3 Hz), 1.29 (3H, s), 1.40-1.92 (10H, m), 2.08-2.40 (4H, m), 2.50 (2H, m), 2.88 (1H, dd, J=13.8, 6 Hz), 3.17 (1H, dd, J=13.8, 10 Hz), 3.25 (1H, m), 3.86 (1H, m), 3.92 (2H, t, J=6.5 Hz), 4.23 (1H, m), 4.67 (1H, dd, J=8, 2 Hz), 5.13 (1H, ddd, J=10, 10, 6 Hz), 5.83 (1H, s), 6.80 (2×1H, d, J=8.5 Hz), 7.12 (2×1H, d, J=8.5 Hz), 7.15 (1H, d, J=10 Hz), 7.46 (1H, d, J=10 Hz), 9.77 (1H, s);

MASS (ES+): m/e 543.41.

Preparation 398

Compound (398) was obtained in a manner similar to Preparation 1.

¹H-NMR (300 MHz, CDCl₃, δ): 0.89 (3H, t, J=7.5 Hz), 1.45 (3×3H, s), 1.53 (3H, s), 1.81-1.96 (2H, m), 5.18 (1H, brs);

MASS (ES+): m/e 218.27.

Preparation 399

Compound (399) was obtained in a manner similar to Preparation 15.

¹H-NMR (300 MHz, CDCl₃, δ): 0.80 (3H, t, J=7.5 Hz), 1.22-2.00 (6H, m), 1.45 (3×3H, s), 1.48 (3H, s), 2.59 (1H, m), 2.91 (1H, dd, J=12.8, 10 Hz), 3.11 (1H, dd, J=12.8, 5 Hz), 3.50 (1H, m), 4.36 (1H, dd, J=8, 4 HZ), 4.94 (1H, ddd, J=10, 8, 5 Hz), 5.11 (1H, d, J=12.5. Hz), 5.16 (1H, d, J=12.5 Hz), 5.16 (1H, s), 6.81 (1H, d, J=8 Hz), 7.16-7.42 (10H, m);

MASS (ES+): m/e 552.36.

Preparation 400

Compound (400) was obtained in a manner similar to Preparation 16.

¹H-NMR (300 MHz, CDCl₃, δ): 0.65 (3×⅓H, t, J=7.3 Hz), 0.98 (3×⅔H, t, J=7.3 Hz), 1.43-2.40 (6H, m), 1.56 (3×⅓H, s), 1.67 (3×⅔H, s), 2.64 (1H, m), 2.98-3.24 (2H, m), 3.60 (1H, m), 4.33 (1H, m), 4.75 (1×⅓H, m), 4.91 (1×⅔H, m), 5.08-5.26 (2H, m), 7.12-7.42 (10+⅔H, m), 7.89 (1×⅓H, d, J=8 Hz), 8.60-8.92 (2H, m);

MASS (ES+): m/e 452.40.

Preparation 401

Compound (401) was obtained in a manner similar to Preparation 16.

¹H-NMR (300 MHz, CDCl₃, δ): 0.72 (3H, t, J=7.3 Hz), 1.38-1.98 (11H, m), 1.44 (3×3H, s), 1.52 (3H, s), 2.29 (1H, m), 2.66 (1H, m), 2.93 (1H, dd, J=13, 9 Hz), 3.07 (1H, dd, J=13, 5.5 Hz), 3.52 (1H, m), 4.08 (1H, m), 4.26-4.42 (3H, m), 4.92 (1H, ddd, J=9, 8, 5.5 Hz), 5.10 (1H, d, J=12 Hz), 5.13 (1H, m), 5.16 (1H, d, J=12 Hz), 6.69 (1H, d, J=8 Hz), 7.01 (1H, s), 7.02-7.40 (10H, m), 7.43 (2×1H, dd, J=7.5, 7.5 Hz), 7.55 (1H, m), 8.03 (2×1H, dd, J=7.5, 1.5 Hz);

MASS (ES+): m/e 785.36.

Preparation 402

Compound (402) was obtained in a manner similar to Preparation 17.

¹H-NMR (300 MHz, CDCl₃, δ): 0.77 (3H, t, J=7.5 Hz), 1.36-2.04 (11H, m), 1.45 (3×3H, s), 1.48 (3H, s), 2.20 (1H, m), 2.64 (1H, m), 2.88-3.13 (2H, m), 3.64 (1H, m), 4.00 (1H, m), 4.26-4.40 (3H, m), 4.88 (1H, m), 5.19 (1H, m), 6.79 (1H, brs), 7.18-7.36 (6H, m), 7.43 (2×1H, dd, J=7.5, 7.5 Hz), 7.56 (1H, m), 8.04 (2×1H, d, J=7.5 Hz);

MASS (ES+): m/e 695.35.

Preparation 403

Compound (403) was obtained in a manner similar to Preparation 18.

¹H-NMR (300 MHz, CDCl₃, δ): 0.53-0.90 (3H, m), 1.35-2.16 (12H, m), 1.46 (3H, s), 2.83-3.19 (3H, m), 3.70 (1H, m), 4.10-4.59 (4H, m), 4.88 (1H, m), 7.10-7.32 (6H, m), 7.40 (2×1H, dd, J=7.5, 7.5 Hz), 7.53 (1H, dd, J=7.5, 7.5 Hz), 8.00 (2×1H, d, J=7.5 Hz), 8.11-8.54 (2H, m);

MASS (ES+): m/e 595.39.

Preparation 404

Compound (404) was obtained in a manner similar to Preparation 76.

¹H-NMR (300 MHz, CDCl₃, δ): 0.94 (3H, t, J=7.3 Hz), 1.37-1.59 (2H, m), 1.61-1.97 (8H, m), 1.73 (3H, s), 2.16 (1H, m), 2.30 (1H, m), 2.95 (1H, dd, J=13.5, 5.5 Hz), 3.20 (1H, m), 3.28 (1H, dd, J=13.5, 10 Hz), 3.88 (1H, m), 4.24 (1H, dt, J=10, 7.5 Hz), 4.32 (2H, t, J=6.3 Hz), 4.67 (1H, dd, J=8, 2 Hz), 5.16 (1H, ddd, J=10, 10, 5 Hz), 5.86 (1H, s), 7.15 (1H, d, J=10 Hz), 7.16-7.33 (5H, m), 7.40-7.50 (3H, m), 7.56 (1H, m), 8.03 (2×1H, d, J=7.5 Hz);

MASS (ES−): m/e 575.46.

Preparation 405

Compound (405) was obtained in a manner similar to Preparation 77.

¹H-NMR (300 MHz, CDCl₃, δ): 0.95 (3H, t, J=7.5 Hz), 1.29-1.93 (1H, m), 1.73 (3H, s), 2.13 (1H, m), 2.30 (1H, m), 2.95 (1H, dd, J=13.5, 5.5 Hz), 3.20 (1H, m), 3.27 (1H, dd, J=13.5, 10 Hz), 3.66 (2H, t, J=6.5 Hz), 3.88 (1H, m), 4.23 (1H, dt, J=10, 7.5 Hz), 4.66 (1H, dd, J=8, 2.5 Hz), 5.15 (1H, ddd, J=10, 10, 5.5 Hz), 5.96 (1H, s), 7.15 (1H, d, J=10 Hz), 7.16-7.32 (5H, m), 7.44 (1H, d, J=10 Hz);

MASS (ES+): m/e 473.38.

Preparation 406

Compound (406) was obtained in a manner similar to Preparation 78. The obtained compound was used in Example 220.

¹H-NMR (300 MHz, CDCl₃, δ): 0.96 (3H, t, J=7.4 Hz), 1.50-1.92 (8H, m), 1.73 (3H, s), 2.17 (1H, m), 2.31 (1H, m), 2.50 (2H, m), 2.95 (1H, dd, J=13.5, 5.5 Hz), 3.19 (1H, m), 3.26 (1H, dd, J=13.5, 10 Hz), 3.88 (1H, m), 4.24 (1H, m), 4.66 (1H, dd, J=7.5, 1.5 Hz), 5.15 (1H, ddd, J=10, 10, 5.5 Hz), 5.90 (1H, s), 7.17 (1H, d, J=10 Hz), 7.17-7.33 (5H, m), 7.39 (1H, d, J=10 Hz), 9.78 (1H, s);

MASS (ES+): m/e 471.39.

Preparation 407

Compound (407) was obtained in a manner similar to Preparation 311.

¹H-NMR (300 MHz, CDCl₃, δ): 0.82 (3H, t, J=7.3 Hz), 1.28 (3H, s), 1.34-1.98 (18H, m), 2.07-2.39 (4H, m), 2.88 (1H, dd, J=13.5, 6 Hz), 3.17 (1H, dd, J=13.5, 10 Hz), 3.26 (1H, m), 3.86 (1H, m), 4.24 (1H, dt, J=10, 7.5 Hz), 4.32 (2H, t, J=6.5 Hz), 4.63-4.74 (2H, m), 5.13 (1H, ddd, J=10, 10, 6 Hz), 5.79 (1H, s), 6.77 (2×1H, d, J=8.8 Hz), 7.11 (2×1H, d, J=8.8 Hz), 7.15 (1H, d, J=10 Hz), 7.44 (2×1H, dd, J=7.5, 7.5 Hz), 7.52 (1H, d, J=10 Hz), 7.56 (1H, m), 7.06 (2×1H, dd, J=7.5, 1 Hz);

MASS (ES+): m/e 661.37.

Preparation 408

Compound (408) was obtained in a manner similar to Preparation 77.

¹H-NMR (300 MHz, CDCl₃, δ): 0.84 (3H, t, J=7.3 Hz), 1.24-1.93 (16H, m), 1.28 (3H, s), 2.08-2.39 (4H, m), 2.87 (1H, dd, J=13.5, 6 Hz), 3.17 (1H, dd, J=13.5, 10 Hz), 3.26 (1H, m), 3.65 (2H, t, J=6.3 Hz), 3.86 (1H, m), 4.22 (1H, dt, J=10.3, 7.7 Hz), 4.67 (1H, dd, J=8, 2.5 Hz), 4.70 (1H, m), 5.13 (1H, ddd, J=10, 10, 6 Hz), 5.90 (1H, S), 6.77 (2×1H, d, J=9 Hz), 7.11 (2×1H, d, J=9 Hz), 7.14 (1H, d, J=10.3 Hz), 7.51 (1H, d, J=10 Hz);

MASS (ES+): m/e 557.44.

Preparation 409

Compound (409) was obtained in a manner similar to Preparation 78. The obtained compound was used in Example 223.

¹H-NMR (300 MHz, CDCl₃, δ): 0.84 (3H, t, J=7.3 Hz), 1.29 (3H, S), 1.49-1.96 (14H, m), 2.07-2.40 (4H, m), 2.50 (2H, m), 2.87 (1H, dd, J=13.5, 6 Hz), 3.17 (1H, dd, J=13.5, 10 Hz), 3.26 (1H, m), 3.86 (1H, m), 4.23 (1H, dt, J=10, 7.5 Hz), 4.63-4.74 (2H, m), 5.13 (1H, ddd, J=10, 10, 6 Hz), 5.87 (1H, S), 6.77 (2×1H, d, J=8.7 Hz), 7.11 (2×1H, d, J=8.7 Hz), 7.16 (1H, d, J=10 Hz), 7.46 (1H, d, J=10 Hz), 9.77 (1H, S);

MASS (ES+): m/e 555.45.

Preparation 410

Compound (410) was obtained in a manner similar to Preparation 311.

¹H-NMR (300 MHz, CDCl₃, δ): 0.82 (3H, t, J=7.3 Hz), 1.28 (3H, s), 1.36-1.99 (8H, m), 2.06-2.39 (4H, m), 2.93 (1H, dd, J=13.5, 6 Hz), 3.21 (1H, dd, J=13.5, 10 Hz), 3.28 (1H, m), 3.85 (1H, m), 4.24 (1H, dt, J=10.3, 7.7 Hz), 4.32 (2H, t, J=6.5 Hz), 4.68 (1H, dd, J=8, 2.5 Hz), 4.74 (2H, S), 5.14 (1H, ddd, J=10, 10, 6 Hz), 5.83 (1H, S), 6.90 (2×1H, d, J=8.8 Hz), 7.10 (1H, d, J=10.3 Hz), 7.22 (2×1H, d, J=8.8 Hz), 7.44 (2×1H, dd, J=7.5, 7.5 Hz), 7.52-7.60 (2H, m), 8.03 (2×1H, d, J=7.5, 1.5 Hz);

MASS (ES−): m/e 666.47(M+Cl).

Preparation 411

Compound (411) was obtained in a manner similar to Preparation 77.

¹H-NMR (300 MHz, CDCl₃, δ): 0.84 (3H, t, J=7.3 Hz), 1.22-1.93 (8H, m), 1.28 (3H, S), 2.08-2.39 (4H, m), 2.89 (1H, dd, J=13.5, 6 Hz), 3.18 (1H, dd, J=13.5, 9.5 Hz), 3.26 (1H, m), 3.65 (2H, t, J=6.5 Hz), 3.80 (3H, S), 3.85 (1H, m), 4.23 (1H, dt, J=10, 7.5 Hz), 4.60 (2H, S), 4.67 (1H, m), 5.13 (1H, ddd, J=10, 9.5, 6 Hz), 5.92 (1H, S), 6.82 (2×1H, d, J=8.5 Hz), 7.12 (1H, d, J=10 Hz), 7.15 (2×1H, d, J=8.5 Hz), 7.53 (1H, d, J=10 Hz);

MASS (ES+): m/e 561.35.

Preparation 412

Compound (412) was obtained in a manner similar to Preparation 78. The obtained compound was used in Example 226.

¹H-NMR (300 MHz, CDCl₃, δ): 0.84 (3H, t, J=7.5 Hz), 1.29 (3H, s), 1.50-1.90 (6H, m), 2.08-2.40 (4H, m), 2.50 (2H, m), 2.89 (1H, dd, J=13.5, 5.5 Hz), 3.18 (1H, dd, J=13.5, 9.5 Hz), 3.26 (1H, m), 3.80 (3H, s), 3.85 (1H, m), 4.23 (1H, m), 4.67 (1H, dd, J=8, 2 Hz), 5.13 (1H, ddd, J=10, 9.5, 5.5 Hz), 5.83 (1H, s), 6.82 (2×1H, d, J=8.5 Hz), 7.13 (1H, d, J=10 Hz), 7.15 (2×1H, d, J=8.5 Hz), 7.47 (1H, d, J=10 Hz), 9.77 (1H,

MASS (ES+): m/e 559.29.

Preparation 413

Compound (413) was obtained in a manner similar to Preparation 342.

¹H-NMR (300 MHz, CDCl₃, δ): 0.82 (3H, t, J=7.3 Hz), 1.28 (3H, s), 1.36-1.98 (8H, m), 2.06-2.40 (4H, m), 2.95 (1H, dd, J=13.5, 6 Hz), 3.23 (1H, dd, J=13.5, 10 Hz), 3.28 (1H, m), 3.86 (1H, m), 4.24 (1H, dt, J=10, 7.7 Hz), 4.32 (2H, t, J=6 Hz), 4.67 (1H, dd, J=8, 2 Hz), 5.18 (1H, m), 5.21 (1H, dd, J=10.5, 1 Hz), 5.71 (1H, dd, J=17.5, 1 Hz), 5.85 (1H, s), 6.67 (1H, dd, J=17.5, 10.5 Hz), 7.13 (1H, dd, J=10 Hz), 7.19 (2×1H, d, J=8.2 Hz), 7.32 (2×1H, d, J=8.2 Hz), 7.44 (2×1H, dd, J=7.5, 7.5 Hz), 7.52-7.60 (2H, m), 8.03 (2×1H, dd, J=7.5, 1.5 Hz);

MASS (ES+): m/e 603.49.

Preparation 414

Compound (414) was obtained in a manner similar to Preparation 77.

¹H-NMR (300 MHz, CDCl₃, δ): 0.84 (3H, t, J=7.3 Hz), 1.24-1.51 (2H, m), 1.28 (3H, s), 1.53-1.94 (6H, m), 2.08-2.40 (4H, m), 2.95 (1H, dd, J=13.5, 6 Hz), 3.22 (1H, dd, J=13.5, 10 Hz), 3.27 (1H, m), 3.65 (2H, d, J=6 Hz), 3.86 (1H, m), 4.23 (1H, dd, J=10, 7.7 Hz), 4.67 (1H, dd, J=8, 2 Hz), 5.18 (1H, ddd, J=10, 10, 6 Hz), 5.22 (1H, dd, J=11, 1 Hz), 5.71 (1H, dd, J=17.5, 1 Hz), 6.00 (1H, s), 6.67 (1H, dd, J=17.5, 11 Hz), 7.13 (1H, d, J=10 Hz), 7.18 (2×1H, d, J=8.2 Hz), 7.32 (2×1H, d, J=8.2 Hz), 7.56 (1H, d, J=10 Hz);

MASS (ES+): m/e 499.58.

Preparation 415

Compound (415) was obtained in a manner similar to Preparation 78. The obtained compound was used in Example 231.

¹H-NMR (300 MHz, CDCl₃, δ): 0.84 (3H, t, J=7.2 Hz), 1.10 (3×3H, s), 1.16-1.32 (4H, m), 1.18 (3H, d, J=7 Hz), 1.28 (3H, s), 1.38-1.62 (3H, m), 1.70-1.86 (3H, m), 2.08-2.39 (4H, m), 2.51 (2H, m), 2.89 (1H, dd, J=13.5, 6 Hz), 3.18 (1H, dd, J=13.5, 9.5 Hz), 3.26 (1H, m), 3.85 (1H, m), 4.12-4.24 (2H, m), 4.49 (2H, ddd, J=5, 1.5, 1.5 Hz), 4.67 (1H, m), 5.13 (1H, ddd, J=10.3, 9.5, 6 Hz), 5.27 (1H, ddt, J=10.3, 1.5, 1.5 Hz), 5.40 (1H, ddt, J=17.2, 1.5, 1.5 Hz), 5.83 (1H, s), 6.04 (1H, ddt, J=17.2, 10.3, 5 Hz), 6.82 (2×1H, d, J=8.6 Hz), 7.08 (1H, d, J=10.2 Hz), 7.14 (2×1H, d, J=8.6 Hz), 7.32-7.48 (6H, m), 7.55 (1H, d, J=10.3 Hz), 7.58-7.67 (4H, m);

MASS (ES+): m/e 837.50.

Preparation 416

Compound (416) was obtained in a manner similar to Preparation 311.

¹H-NMR (300 MHz, CDCl₃, δ): 0.81 (3H, t, J=7.5 Hz), 1.27 (3H, s), 1.36-1.55 (2H, m), 1.64-1.98 (6H, m), 2.06-2.39 (4H, m), 2.89 (1H, dd, J=13.5, 6.5 Hz), 3.18 (1H, dd, J=13.5, 10 Hz), 3.26 (1H, m), 3.85 (1H, m), 4.24 (1H, dt, J=10.3, 7.5 Hz), 4.31 (2H, t, J=6.5 Hz), 4.67 (1H, m), 5.13 (1H, m), 5.17 (2H, s), 5.80 (1H, s), 6.90 (2×1H, d, J=8.5 Hz), 7.13 (1H, d, J=10.3 Hz), 7.15 (2×1H, d, J=8.5 Hz), 7.22 (1H, m), 7.44 (2×1H, dd, J=7.5, 7.5 Hz), 7.48-7.60 (3H, m), 7.71 (1H, m), 8.03 (2×1H, dd, J=7.5, 1.5 Hz), 8.59 (1H, d, J=4.5 Hz);

MASS (ES+): m/e 684.34.

Preparation 417

Compound (417) was obtained in a manner similar to Preparation 77.

¹H-NMR (300 MHz, CDCl₃, δ): 0.83 (3H, t, J=7.3 Hz), 1.28 (3H, s), 1.30-1.94 (9H, m), 2.08-2.40 (4H, m), 2.89 (1H, dd, J=13.5, 6 Hz), 3.18 (1H, dd, J=13.5, 10 Hz), 3.26 (1H, m), 3.66 (2H, dt, J=6, 5 Hz), 3.85 (1H, m), 4.22 (1H, dt, J=10, 7.5 Hz), 4.67 (1H, dd, J=8, 2 Hz), 5.13 (1H, ddd, J=10, 10, 6 Hz), 5.17 (2H, s), 5.86 (1H, s), 6.90 (2×1H, d, J=8.7 Hz), 7.12 (1H, d, J=10 Hz), 7.14 (2×1H, d, J=8.7 Hz), 7.22 (1H, dd, J=7.5, 5 Hz), 7.50 (1H, d, J=7.5 Hz), 7.52 (1H, d, J=10 Hz), 7.71 (1H, ddd, J=7.5, 7.5, 2 Hz), 8.58 (1H, dd, J=5, 2 Hz);

MASS (ES+): m/e 579.69.

Preparation 418

Compound (418) was obtained in a manner similar to Preparation 78. The obtained compound was used in Example 234.

¹H-NMR (300 MHz, CDCl₃, δ): 0.84 (3H, t, J=7.5 Hz), 1.29 (3H, s), 1.50-1.92 (6H, m), 2.08-2.40 (4H, m), 2.50 (2H, m), 2.89 (1H, dd, J=13.5, 6 Hz), 3.17 (1H, dd, J=13.5, 10 Hz), 3.25 (1H, m), 3.85 (1H, m), 4.23 (1H, m), 4.67 (1H, dd, J=8, 2 Hz), 5.13 (1H, ddd, J=10, 10, 6 Hz), 5.17 (2H, s), 5.88 (1H, s), 6.90 (2×1H, d, J=8.7 Hz), 7.11-7.18 (3H, m), 7.22 (1H, dd, J=7.5, 5 Hz), 7.47 (1H, d, J=10 Hz), 7.50 (1H, d, J=7.5 Hz), 7.71 (1H, ddd, J=7.5, 7.5, 1.8 Hz), 8.59 (1H, dd, J=5, 1.8 Hz), 9.77 (1H, t, J=1 Hz);

MASS (ES+): m/e 578.36.

Preparation 419

To a 0.5 M solution of isopropenyl magnesium bromide in tetrahydrofuran (61.4 ml) was added a solution of tributyltin chloride (2.5 g) in tetrahydrofuran (8.0 ml) and the mixture was gently refluxed overnight. The reaction mixture was cooled to ambient temperature. The reaction was quenched by addition of an aqueous saturated ammonium chloride to the mixture. To the reaction mixture was added ice and extracted with hexane. The extract was washed with water and saturated brine and dried over magnesium sulfate. The magnesium sulfate was filtered off and the extract was evaporated to give the objective Compound (419) as an oil.

¹H-NMR (300 MHz, CDCl₃, δ): 0.89 (3×3H, t, J=7 Hz), 1.24-1.60 (18H, m), 5.08 (1H, m), 5.69 (1H, m).

Preparation 420

Compound (420) was obtained in a manner similar to Preparation 342.

¹H-NMR (300 MHz, CDCl₃, δ): 0.82 (3H, t, J=7.3 Hz), 1.28 (3H, s), 1.38-1.53 (2H, m), 1.65-1.99 (6H, m), 2.06-2.40 (4H, m), 2.12 (3H, s), 2.96 (1H, dd, J=13.5, 6 Hz), 3.24 (1H, dd, J=13.5, 9.5 Hz), 3.29 (1H, m), 3.87 (1H, m), 4.24 (1H, dt, J=10.5, 7.5 Hz), 4.32 (2H, t, J=6.5 Hz), 4.68 (1H, dd, J=8, 2 Hz), 5.05 (1H, brs), 5.19 (1H, m), 5.35 (1H, s), 5.90 (1H, s), 7.14 (1H, d, J=10.5 Hz), 7.19 (2×1H, d, J=8.3 Hz), 7.38 (2×1H, d, J=8.3 Hz), 7.44 (2×1H, dd, J=7.5, 7.5 Hz), 7.52-7.61 (2H, m), 8.03 (2×1H, dd, J=7.5, 1.5 Hz);

MASS (ES+): m/e 617.51.

Preparation 421

Compound (421) was obtained in a manner similar to Preparation 77.

¹H-NMR (300 MHz, CDCl₃, δ): 0.84 (3H, t, J=7.3 Hz), 1.24-1.94 (8H, m), 1.29 (3H, s), 2.08-2.40 (4H, m), 2.12 (3H, s), 2.96 (1H, dd, J=13.5, 6 Hz), 3.24 (1H, dd, J=13.5, 10 Hz), 3.29 (1H, m), 3.66 (2H, t, J=6.5 Hz), 3.87 (1H, m), 4.24 (1H, dt, J=10.3, 8 Hz), 4.69 (1H, dd, J=7.5, 1.5 Hz), 5.05 (1H, brs), 5.19 (1H, ddd, J=10, 10, 6 Hz), 5.35 (1H, s), 6.01 (1H, s), 7.15 (1H, d, J=10.3 Hz), 7.19 (2×1H, d, J=8 Hz), 7.38 (2×1H, d, J=8 Hz), 7.57 (1H, d, J=10 Hz);

MASS (ES+): m/e 513.56.

Preparation 422

Compound (422) was obtained in a manner similar to Preparation 78. The obtained compound was used in Example 237.

¹H-NMR (300 MHz, CDCl₃, δ): 0.84 (3H, t, J=7.3 Hz), 1.29 (3H, s), 1.50-1.92 (6H, m), 2.08-2.40 (4H, m), 2.12 (3H, s), 2.50 (2H, m), 2.96 (1H, dd, J=13.5, 6.3 Hz), 3.23 (1H, dd, J=13.5, 9.5 Hz), 3.28 (1H, m), 3.87 (1H, m), 4.24 (1H, m), 4.68 (1H, dd, J=7.5, 2 Hz), 5.05 (1H, s), 5.18 (1H, ddd, J=10.3, 9.5, 6.3 Hz), 5.35 (1H, s), 5.95 (1H, s), 7.15 (1H, d, J=10.3 Hz), 7.19 (2×1H, d, J=8.3 Hz), 7.38 (2×1H, d, J=8.3 Hz), 7.51 (1H, d, J=10.3 Hz), 9.77 (1H, s);

MASS (ES+): m/e 511.53.

Preparation 423

Compound (423) was obtained in a manner similar to Example 147 mentioned below.

¹H-NMR (300 MHz, CDCl₃, δ): 0.82 (3H, t, J=7.3 Hz), 1.27 (3H, s), 1.36-1.55 (2H, m), 1.60-1.98 (6H, m), 2.08-2.40 (4H, m), 2.96 (1H, dd, J=13.5, 6.3 Hz), 3.23 (1H, dd, J=13.5, 9.5 Hz), 3.28 (1H, m), 3.86 (1H, m), 4.25 (1H, dt, J=10.3, 7.7 Hz), 4.32 (1H, t, J=6.7 Hz), 4.62-4.71 (3H, m), 5.18 (1H, m), 5.92 (1H, s), 7.13 (1H, d, J=10.3 Hz), 7.23 (2×1H, d, J=8.5 Hz), 7.28 (2×1H, d, J=8.5 Hz), 7.44 (2×1H, dd, J=7.5, 7.5 Hz), 7.52-7.62 (2H, m), 8.03 (2×1H, dd, J=7.5, 1.5 Hz);

MASS (ES+): m/e 607.53.

Preparation 424

To a solution of the Compound (420) in N,N-dimethylformamide (5 ml) were added imidazole (57.9 mg) and then tert-butyldimethylsilyl chloride (103 mg), and the mixture was stirred at ambient temperature for 1 day. To the reaction mixture were added additional imidazole (116 mg), tert-butyldimethylsilyl chloride (210 mg) and 4-(dimethylamino)pyridine (100 mg) and the mixture was stirred at ambient temperature for 4 hours. The mixture was poured into water and extracted with ethyl acetate. The extract was washed with saturated brine (×2), dried over sodium sulfate, evaporated and purified by preparative thin layer chromatography (eluting with ethyl acetate/hexane=1/1) to give the objective Compound (424) as a white foam.

¹H-NMR (300 MHz, CDCl₃, δ): 0.08 (2×3H, s), 0.82 (3H, t, J=7.4 Hz), 0.93 (3×3H, s), 1.28 (3H, s), 1.36-1.56 (2H, m), 1.58-2.00 (6H, m), 2.08-2.40 (4H, m), 2.94 (1H, dd, J=13.5, 6.3 Hz), 3.18-3.31 (2H, m), 3.86 (1H, m), 4.24 (1H, dt, J=10.2, 7.7 Hz), 4.32 (2H, t, J=6.5 Hz), 4.67 (1H, m), 4.69 (2H, s), 5.17 (1H, m), 5.89 (1H, s), 7.13 (1H, d, J=10.2 Hz), 7.19 (2×1H, d, J=8.4 Hz), 7.23 (2×1H, d, J=8.4 Hz), 7.44 (2×1H, dd, J=7.5, 7.5 Hz), 7.52-7.60 (2H, m), 8.03 (2×1H, dd, J=7.5, 1.5 Hz);

MASS (ES+): m/e 721.50.

Preparation 425

Compound (425) was obtained in a manner similar to Preparation 77.

¹H-NMR (300 MHz, CDCl₃, δ): 0.08 (2×3H, s), 0.84 (3H, t, J=7.4 Hz), 0.93 (3×3H, s), 1.28 (3H, s), 1.30-1.94 (8H, m), 2.08-2.40 (4H, m), 2.94 (1H, dd, J=13.5, 6 Hz), 3.17-3.31 (2H, m), 3.65 (2H, t, J=6.5 Hz), 3.86 (1H, m), 4.23 (1H, dt, J=10.2, 7.7 Hz), 4.67 (1H, m), 4.69 (2H, s), 5.18 (1H, m), 5.91 (1H, s), 7.13 (1H, d, J=10.2 Hz), 7.19 (2×1H, d, J=8.5 Hz), 7.22 (2×1H, d, J=8.5 Hz), 7.53 (1H, d, J=10.2 Hz);

MASS (ES+): m/e 617.61.

Preparation 426

Compound (426) was obtained in a manner similar to Preparation 78. The obtained compound was used in Example 240.

¹H-NMR (300 MHz, CDCl₃, δ): 0.08 (2×3H, s), 0.84 (3H, t, J=7.3 Hz), 0.93 (3×3H, s), 1.29 (3H, s), 1.52-1.92 (6H, m), 2.08-2.40 (4H, m), 2.50 (2H, m), 2.94 (1H, dd, J=13.5, 6 Hz), 3.22 (1H, dd, J=13.5, 9.5 Hz), 3.25 (1H, m), 3.86 (1H, m), 4.23 (1H, m), 4.67 (1H, m), 4.69 (2H, s), 5.17 (1H, ddd, J=10.3, 9.5, 6 Hz), 5.90 (1H, s), 7.16 (1H, d, J=10 Hz), 7.18 (2×1H, d, J=8 Hz), 7.23 (2×1H, d, J=8 Hz), 7.49 (1H, d, J=10.3 Hz), 9.77 (1H, t, J=1 Hz);

MASS (ES+): m/e 615.60.

Preparation 427

Compound (427) was obtained in a manner similar to Preparation 77.

¹H-NMR (300 MHz, DMSO-d₆, δ): 1.12-1.43 (2H, m), 1.36 (9×½H, s), 1.39 (9×½H, s), 1.48-1.67 (3H, m), 2.06 (1H, m), 2.77 (½H, m), 2.93 (½H, m), 3.80 (1H, m), 4.53 (½H, m), 4.62 (½H, m), 12.75 (1H, br);

MASS (ES−): m/e 228.51.

Preparation 428

Compound (428) was obtained in a manner similar to Preparation 119.

¹H-NMR (300 MHz, CDCl₃, δ): 1.24 (3H, t, J=7 Hz), 1.40 (3H, t, J=7 Hz), 3.03 (1H, dd, J=14, 6 Hz), 3.07 (1H, dd, J=14, 5 Hz), 3.99 (2H, q, J=7 Hz), 4.16 (2H, q, J=7 Hz), 4.59 (1H, ddd, J=7.5, 6, 5 Hz), 5.09 (1H, d, J=12 Hz), 5.11 (1H, d, J=12 Hz), 5.21 (1H, d, J=7.5 Hz), 6.79 (2×1H, d, J=9 Hz), 7.00 (2×1H, d, J=9 Hz), 7.27-7.40 (5H, m);

MASS (ES+): m/e 372.52.

Preparation 429

Compound (429) was obtained in a manner similar to Preparation 77.

¹H-NMR (300 MHz, CDCl₃, δ): 1.38 (3H, t, J=7 Hz), 3.07 (2H, m), 3.96 (2H, q, J=7 Hz), 4.60 (1H, m), 5.04 (1H, d, J=12 Hz), 5.08 (1H, d, J=12 Hz), 5.23 (1H, br), 6.77 (2×1H, d, J=7.5 Hz), 7.03 (2×1H, d, J=7.5 Hz), 7.20-7.40 (5H, m);

MASS (ES−): m/e 342.57.

Preparation 430

Compound (430) was obtained in a manner similar to Preparation 14.

¹H-NMR (300 MHz, CDCl₃, δ): 1.30-1.64 (8H, m), 1.47 (3×3H, s), 1.65-1.82 (3H, m), 1.95 (1H, m), 2.25 (1H, m), 2.72 (1H, m), 4.25 (2H, t, J=6.3 Hz), 4.67 (1H, m), 4.75 (1H, m), 5.14 (1H, d, J=12.3 Hz), 5.18 (1H, d, J=12.3 Hz), 6.63 (1H, br), 7.30-7.38 (5H, m), 7.43 (2×1H, dd, J=7.5 Hz), 7.56 (1H, m), 8.02 (2×1H, dd, J=7.5, 1.5 Hz);

MASS (ES+): m/e 553.50.

Preparation 431

Compound (431) was obtained in a manner similar to Preparation 15.

¹H-NMR (300 MHz, DMSO-d₆, δ): 1.36-1.93 (11H, m), 2.07 (1H, m), 2.89 (1H, m), 3.19 (1H, m), 3.79 (1H, m), 4.25 (2H, t, J=6.3 Hz), 4.38 (1H, m), 5.12 (2H, s), 7.30-7.42 (5H, m), 7.53 (2×1H, dd, J=7.5, 7.5 Hz), 7.67 (1H, m), 7.97 (2×1H, dd, J=7.5, 1.5 Hz), 8.94 (1H, d, J=7.5 Hz);

MASS (ES+): m/e 453.52.

Preparation 432

Compound (432) was obtained in a manner similar to Preparation 15.

¹H-NMR (300 MHz, CDCl₃, δ): 0.81-0.99 (6H, m), 1.00-2.06 (13H, m), 1.37 (9×⅖H, s), 1.44 (9×⅗H, s), 2.20 (1H, m), 2.46 (1H, m), 3.12 (1H, m), 3.87 (1H, m), 4.15 (1H, m), 4.26 (2H, m), 4.46-4.66 (2.2H, m), 5.04 (0.4H, d, J=7.8 Hz), 5.12 (1H, d, J=12.3. Hz), 5.18 (1H, d, J=12.3 Hz), 5.20-5.29 (1H, m), 6.48 (0.6H, d, J=7.7 Hz), 7.28-7.38 (5H, m), 7.40-7.48 (2H, m), 7.56 (1H, m), 7.98-8.05 (2H, m), 8.28 (0.4H, d, J=7.8 Hz);

MASS (ES+): m/e 666.58.

Preparation 433

Compound (433) was obtained in a manner similar to Preparation 16.

¹H-NMR (300 MHz, CDCl₃, δ): 0.90 (3H, m), 1.11 (3H, m), 1.20-2.68 (16H, m), 3.52 (0.5H, m), 3.68 (0.5H, m), 4.24 (2H, m), 4.37-4.62 (2H, m), 5.09 (1H, d, J=12.3 Hz), 5.15 (0.5H, m), 5.18 (1H, d, J=12.3 Hz), 5.36 (0.5H, m), 7.15 (0.5H, m), 7.24-7.37 (5.5H, m), 7.41 (2×1H, dd, J=7.5, 7.5 Hz), 7.54 (1H, m), 8.00 (2×1H, d, J=7.5 Hz), 8.33 (2H, br);

MASS (ES+): m/e 566.60.

Preparation 434

Compound (434) was obtained in a manner similar to Preparation 16.

¹H-NMR (300 MHz, CDCl₃, δ): 0.66-0.92 (6H, m), 1.10-1.98 (13H, m), 1.39 (3H, t, J=7 Hz), 2.16 (1H, m), 2.47 (1H, m), 2.86-3.04 (2H, m), 3.18 (1H, m), 3.87 (1H, m), 3.91-4.02 (2H, m), 4.22 (2H, t, J=6.5 Hz), 4.30-4.64 (3H, m), 4.82 (0.5H, dd, J=8.5, 6 Hz), 4.99-5.24 (4.5H, m), 5.34 (0.5H, d, J=7 Hz), 5.60 (0.5H, br), 6.40-6.68 (1.5H, m), 6.74-6.82 (2H, m), 7.00-7.12 (2H, m), 7.24-7.48 (12H, m), 7.55 (1H, m), 7.96-8.04 (2H, m), 8.14 (0.5H, d, J=6 Hz);

MASS (ES+): m/e 891.42.

Preparation 435

Compound (435) was obtained in a manner similar to Preparation 53.

¹H-NMR (300 MHz, CDCl₃, δ): 0.62-0.84 (6H, m), 1.00-2.03 (14H, m), 1.35 (3H, t, J=7 Hz), 2.50-2.66 (3H, m), 3.07 (1H, m), 3.34 (1H, m), 3.92 (2H, q, J=7 Hz), 4.23 (2H, t, J=6.2 Hz), 4.36-4.86 (4H, m), 6.77 (2×1H, d, J=8.3 Hz), 7.14-7.28 (1H, br), 7.20 (2×1H, d, J=8.3 Hz), 7.40 (2×1H, dd, J=7.5, 7.5 Hz), 7.48-7.70 (2H, m), 7.99 (2×1H, dd, J=7.5, 1.5 Hz);

MASS (ES+): m/e 667.55.

Preparation 436

Compound (436) was obtained in a manner similar to Preparation 76.

¹H-NMR (300 MHz, CDCl₃, δ): 0.68-0.78 (3H, m), 0.78 (3H, d, J=6.5 Hz), 1.04-1.98 (14H, m), 1.38 (3H, t, J=7 Hz), 2.45 (1H, m), 2.69 (1H, m), 2.80 (1H, dd, J=14, 6 Hz), 3.18 (1H, dd, J=14, 10 Hz), 3.95 (2H, q, J=7 Hz), 4.31 (2H, t, J=6.5 Hz), 4.45-4.64 (4H, m), 4.86 (1H, m), 5.85-6.10 (2H, br), 6.22 (1H, d, J=11 Hz), 6.74 (2×1H, d, J=8.5 Hz), 7.08 (2×1H, d, J=8.5 Hz), 7.43 (2×1H, dd, J=7.5, 7.5 Hz), 7.56 (1H, dddd, J=7.5, 7.5, 1.5, 1.5 Hz), 8.02 (2×1H, dd, J=7.5, 1.5 Hz);

MASS (ES+): m/e 649.51.

Preparation 437

Compound (437) was obtained in a manner similar to Preparation 77.

¹H-NMR (500 MHz, CDCl₃, δ): 0.67-0.78 (3H, m), 0.79 (3H, d, J=6 Hz), 0.83-1.96 (14H, m), 1.38 (3H, t, J=7 Hz), 2.46 (1H, m), 2.75 (1H, m), 2.80 (1H, dd, J=13.5, 6 Hz), 3.18 (1H, dd, J=13.5, 10 Hz), 3.62 (2H, t, J=6.2 Hz), 3.94 (2H, q, J=7 Hz), 4.46-4.65 (4H, m), 4.93 (1H, brd, J=5 Hz), 6.17 (1H, br), 6.44 (1H, br), 6.46 (1H, d, J=10.5 Hz), 6.71 (2×1H, d, J=8.5 Hz), 7.05 (2×1H, d, J=8.5 Hz);

MASS (ES+): m/e 545.50.

Preparation 438

Compound (438) was obtained in a manner similar to Preparation 78. The obtained compound was used in Example 243.

¹H-NMR (300 MHz, CDCl₃, δ): 0.67-0.88 (3H, m), 0.79 (3H, d, J=6.7 Hz), 1.09 (1H, m), 1.20-1.47 (3H, m), 1.37 (3H, t, J=7 Hz), 1.54-1.98 (8H, m), 2.46 (1H, m), 2.51 (2H, t, J=7 Hz), 2.74 (1H, m), 2.80 (1H, dd, J=14, 6 Hz), 3.16 (1H, dd, J=14, 10 Hz), 3.92 (2H, q, J=7 Hz), 4.48-4.68 (4H, m), 4.94 (1H, m), 6.21 (1H, br), 6.45 (1H, d, J=10.3 Hz), 6.46 (1H, br), 6.70 (2×1H, d, J=8.8 Hz), 7.05 (2×1H, d, J=8.8 Hz), 9.75 (1H, s);

MASS (ES+): m/e 543.58.

Preparation 439

Compound (439) was obtained in a manner similar to Preparation 15.

¹H-NMR (300 MHz, CDCl₃, δ): 1.30 (3H, d, J=7 Hz), 1.35-1.60 (1H, m), 1.46 (9H, s), 1.71-2.01 (3H, m), 2.62-2.74 (1H, m), 2.94 (1H, dd, J=13, 9 Hz), 3.06 (1H, dd, J=13, 6 Hz), 3.46-3.65 (1H, m), 4.00-4.25 (1H, m), 4.38 (1H, dd, J=8, 4 Hz), 4.95 (1H, ddd, J=9, 8, 1 Hz), 5.09 (1H, d, J=12 Hz), 5.20 (1H, d, J=12 Hz), 5.20 (1H, d, J=7 Hz), 6.81 (1H, d, J=8 Hz), 7.16-7.40 (10H, m);

MASS: m/z 524.38 (M+H)⁺.

Preparation 440

Compound (440) was obtained in a manner similar to Preparation 16.

¹H-NMR (300 MHz, CDCl₃, δ): 1.17-2.03 (10H, m), 1.3.0 (3H, d, J=7 Hz), 1.43 (9H, s), 2.71-2.86 (1H, m), 3.00 (2H, d, J=7 Hz), 3.51-3.64 (1H, m), 4.01-4.18 (1H, m), 4.31 (2H, t, J=6 Hz), 4.33-4.40 (1H, m), 4.47 (1H, t, J=7 Hz), 4.93 (1H, dt, J=8, 7 Hz), 5.04 (1H, d, J=12 Hz), 5.18 (1H, d, J=12 Hz), 5.18 (1H, d, J=10 Hz), 6.66-6.84 (1H, m), 6.74 (1H, d, J=8 Hz), 7.15-7.38 (10H, m), 7.38-7.48 (2H, m), 7.51-7.60 (1H, m), 8.03 (2H, d, J=8 Hz);

MASS: m/z 757.27 (M+H)⁺.

Preparation 441.

Compound (441) was obtained in a manner similar to Preparation 17.

¹H-NMR (300 MHz, CDCl₃, δ): 1.12-2.11 (10H, m), 1.25 (3H, d, J=7 Hz), 1.43 (9H, s), 2.88-3.00 (1H, m), 2.99-3.17 (2H, m), 3.82-3.92 (1H, m), 4.01-4.23 (2H, m), 4.35-4.48 (1H, m), 5.01 (1H, dt, J=8, 7 Hz), 5.25-5.34 (1H, m), 7.15-7.35 (6H, m), 7.43 (2H, t, J=8 Hz), 7.55 (1H, t, J=8 Hz), 8.03 (2H, d, J=8 Hz), 8.25-8.35 (1H, m);

MASS: m/z 667.29 (M+H)⁺.

Preparation 442

Compound (442) was obtained in a manner similar to Preparations 18 and 76.

¹H-NMR (300 MHz, CDCl₃, δ): 1.27 (3H, d, J=7-Hz), 1.36-1.57 (2H, m), 1.64-1.99 (7H, m), 2.13-2.26 (1H, m), 2.26-2.38 (1H, m), 2.93 (1H, dd J=14, 10 Hz), 3.16 (1H, dt, J=10, 7 Hz), 3.22 (1H, dd, J=14, 10 Hz), 3.90 (1H, dt, J=10, 4 Hz), 4.31 (2H, t, J=7 Hz), 4.52-4.69 (2H, m), 5.12 (1H, dt, J=6, 10 Hz), 6.11 (1H, d, J=10 Hz), 6.54 (1H, d J=11 Hz), 7.14-7.34 (5H, m), 7.17 (1H, d, J=10 Hz), 7.44 (1H, dd, J=8, 7 Hz), 7.56 (1H, t, J=7 Hz), 8.03 (2H, d, J=8 Hz);

MASS: m/z 549.35 (M+H)⁺.

Preparation 443

Compound (443) was obtained in a manner similar to Preparation 77.

H-NMR (300 MHz, CDCl₃, δ): 1.22-1.52 (2H, m), 1.28 (3H, d, J=7 Hz), 1.54-1.96 (7H, m), 2.12-2.27 (1H, m), 2.28-2.39 (1H, m), 2.93 (1H, dd J=14, 6 Hz), 3.16 (1H, dt, J=10, 7 Hz), 3.21 (1H, dd, J=14, 10 Hz), 3.61-3.72 (1H, m), 3.90 (1H, dt, J=10, 4 Hz), 4.30 (2H, t, J=7 Hz), 4.51-4.62 (2H, m), 4.61-4.69 (1H, m), 5.11° (1H, dt, J=6, 10 Hz), 6.36 (1H, d, J=, 10 Hz), 6.60 (1H, d, J=10 Hz), 7.16-7.34 (6H, m);

MASS: m/z 445.36 (M+H)⁺.

Preparation 444

Compound (444) was obtained in a manner similar to Preparation 78. The obtained compound was used in Example 246.

¹H-NMR (300 MHz, CDCl₃, δ): 1.29 (3H, d, J=7 Hz), 1.49-1.96 (6H, m), 2.10-2.41 (2H, m), 2.43-2.57 (2H, m), 2.93 (1H, dd J=14, 6 Hz), 3.15 (1H, dt, J=10, 7 Hz), 3.21 (1H, dd, J=14, 10 Hz), 3.90 (1H, dt, J=10, 4 Hz), 4.30 (2H, dt, J=10, 7 Hz), 4.52-4.69 (2H, m), 5.11 (1H, dt, J=6, 10 Hz), 6.16 (1H, d, J=10 Hz), 6.53 (1H, d, J=10 Hz), 7.13-7.33 (6H, m), 9.77 (1H, s);

MASS: m/z 443.37 (M+H)⁺.

Preparation 445

Compound (445) was obtained in a manner similar to Preparation 13.

¹H-NMR (300 MHz, CDCl₃, δ): 1.25 (3H, s), 1.41 (6H, s), 2.90-3.35 (2H, m), 4.59-4.71 (0.5H, m), 4.89-5.01 (0.5H, m), 7.28-7.38 (2H, m), 7.57 (2H, d, J=8.1 Hz);

MASS (ES−): m/e 332.13 (M−1).

Preparation 446

Compound (446) was obtained in a manner similar to Preparation 14.

¹H-NMR (300 MHz, CDCl₃, δ): 1.30 (2H, s), 1.40 (7H, s), 1.51-1.70 (1H, m), 1.81-2.07 (3H, m), 2.73-3.09 (3H, m), 3.54-3.66 (1H, m), 4.32-4.43 (1H, m), 4.59-4.74 (1H, m), 5.05-5.27 (2H, m), 5.27-5.37 (1H, m), 7.22-7.42 (7H, m), 7.42-7.59 (2H, m);

MASS (ES+): m/e 521.33 (M+1).

Preparation 447

Compound (447) was obtained in a manner similar to Preparation 15.

¹H-NMR (300 MHz, CDCl₃, δ): 0.80 (3H, t, J=7.5 Hz), 1.38 (3H, s), 1.43 (9H, s), 1.50-1.68 (1H, m), 1.74-2.03 (5H, m), 2.80-2.95 (1H, m), 3.01-3.14 (2H, m), 3.51-3.68 (1H, m), 4.34-4.41 (1H, m), 4.92-5.02 (1H, m), 5.10 (1H, d, J=12.4 Hz), 5.17 (1H, d, J=12.4 Hz), 6.82-6.91 (0.6H, m), 7.12-7.18 (0.4H, m), 7.12-7.40 (7H, m), 7.40-7.57 (2H, m);

MASS (ES+): m/e 620.33 (M+1).

Preparation 448

Compound (448) was obtained in a manner similar to Preparation 16.

¹H-NMR (300 MHz, CDCl₃, δ): 0.73 (3H, t, J=7.3 Hz), 1.39-2.02 (11H, m), 1.43 (3H, s), 1.44 (6H, s), 1.46 (3H, s), 2.07-2.34 (1H, m), 2.86-3.16 (3H, m), 3.49-3.68 (1H, m), 3.90-4.13 (1H, m), 4.30-4.42 (1H, m), 4.31 (2H, t, J=6.2 Hz), 4.93-5.19 (4H, m), 6.79-6.93 (1H, m), 7.29-7.37 (7H, m), 7.39-7.48 (3H, m), 7.49-7.60 (3H, m), 8.00-8.05 (2H, m);

MASS (ES+): m/e 853.22 (M+1).

Preparation 449

Compound (449) was obtained in a manner similar to Preparation 17.

¹H-NMR (300 MHz, CDCl₃, δ): 0.73 (3H, t, J=7.3 Hz), 1.18-2.25 (11H, m), 1.41 (3H, s), 1.44 (9H, s), 2.93-3.20 (2H, m), 3.68-3.82 (1H, m), 3.94-4.07 (1H, m), 4.07-4.20 (1H, m), 4.27-4.43 (3H, m), 4.94-5.10 (1H, m), 5.34 (1H, brs), 6.82 (1H, s), 7.33-7.49 (5H, m), 7.50-7.61 (3H, m), 8.00-8.09 (2H, m);

MASS (ES+): m/e 763.26 (M+1).

Preparation 450

Compound (450) was obtained in a manner similar to Preparation 18.

¹H-NMR (300 MHz, CDCl₃, δ): 0.59-0.73 (3H, m), 1.38 (3H, s), 1.54-2.20 (12H, m), 2.91-3.22 (3H, m), 3.69-3.82 (1H, m), 4.18-4.41 (4H, m), 4.94-5.08 (1H, m), 7.29-7.58 (7H, m), 7.66-7.82 (1H, m), 7.94-8.05 (2H, m), 8.14-8.43 (2H, m);

MASS (ES+): m/e 662.30 (Free).

Preparation 451

Compound (451) was obtained in a manner similar to Preparation 76.

¹H-NMR (300 MHz, CDCl₃, δ): 0.80 (3H, t, J=7.3 Hz), 1.28 (3H, s), 1.36-2.00 (8H, m), 2.02-2.38 (4H, m), 2.98-3.14 (1H, m), 3.22-3.36 (2H, m), 3.79-3.92 (1H, m), 4.18-4.32 (1H, m), 4.32 (2H, t, J=6.6 Hz), 4.63-4.73 (1H, m), 5.13-5.26 (1H, m), 5.82 (1H, s), 7.05 (1H, d, J=10.6 Hz), 7.31-7.40 (2H, m), 7.42 (1H, d, J=8.4 Hz), 7.46 (1H, d, J=8.4 Hz), 7.50-7.67 (4H, m), 8.03 (2H, d, J=8.4 Hz);

MASS (ES+): m/e 645.27 (M+1).

Preparation 452

Compound (452) was obtained in a manner similar to Preparation 77.

¹H-NMR (300 MHz, CDCl₃, δ): 0.82 (3H, t, J=7.3 Hz), 1.21-1.95 (8H, m), 1.29 (3H, s), 2.07-2.42 (4H, m), 3.05 (1H, dd, J=13.5, 6.6 Hz), 3.24-3.37 (1H, m), 3.28 (1H, dd, J=13.5, 9.2 Hz), 3.66 (2H, t, J=6.3 Hz), 3.80-3.91 (1H, m), 4.19-4.30 (1H, m), 4.66-4.73 (1H, m), 5.14-5.26 (1H, m), 5.92 (1H, s), 7.05 (1H, d, J=10.3 Hz), 7.35 (2H, d, J=8.2 Hz), 7.54 (2H, d, J=8.2 Hz), 7.62 (1H, d, J=10.3 Hz);

MASS (ES+): m/e 541.28 (M+1).

Preparation 453

Compound (453) was obtained in a manner similar to Preparation 78. The obtained compound was used in Example 254.

¹H-NMR (300 MHz, CDCl₃, δ): 0.82 (3H, t, J=7.3 Hz), 1.29 (3H, s), 1.49-1.92 (6H, m), 2.09-2.41 (4H, m), 2.51 (2H, t, J=7.3 Hz), 3.04 (1H, dd, J=13.2, 6.6 Hz), 3.25-3.36 (1H, m), 3.28 (1H, dd, J=13.2, 9.9 Hz), 3.81-3.92 (1H, m), 4.18-4.31 (1H, m), 4.65-4.74 (1H, m), 5.14-5.26 (1H, m), 5.85 (1H, s), 7.06 (1H, d, J=10.3 Hz), 7.35 (2H, d, J=8.3 Hz), 7.53-7.63 (1H, m), 7.54 (2H, d, J=8.3 Hz), 9.77 (1H, s);

MASS (ES+): m/e 539.31 (M+1).

Preparation 454

Compound (454) was obtained in a manner similar to Preparation 13.

¹H-NMR (300 MHz, CDCl₃, δ): 1.48 (9H, s), 1.56 (3H, s), 3.21-3.38 (2H, m), 5.05 (1H, brs), 7.10-7.21 (2H, m), 7.22-7.35 (3H, m);

MASS (ES+): m/e 280.14 (M+1).

Preparation 455

Compound (455) was obtained in a manner similar to Preparation 15.

¹H-NMR (300 MHz, CDCl₃, δ): 1.36-1.70 (2H, m), 1.41 (2H, s), 1.42 (3H, s), 1.45 (7H, s), 1.73-1.98 (2H, m), 2.57-2.68 (1H, m), 2.82-3.00 (1H, m), 3.01-3.28 (3H, m), 3.48-3.62 (1H, m), 4.32-4.40 (1H, m), 4.74-5.01 (2H, m), 5.10 (1H, d, J=13.6 Hz), 5.16 (1H, d, J=13.6 Hz), 6.67-7.00 (1H, m), 7.05-7.40 (15H, m);

MASS (ES+): m/e 614.39 (M+1).

Preparation 456

Compound (456) was obtained in a manner similar to Preparation 16.

¹H-NMR (300 MHz, CDCl₃, δ): 1.11-2.13 (10H, m), 1.44 (9H, s), 1.55 (3H, s), 2.60-2.73 (1H, m), 2.85-3.29 (4H, m), 3.57-3.70 (1H, m), 3.88-4.16 (1H, m), 4.19-4.41 (3H, m), 4.91-5.02 (1H, m), 5.03-5.33 (3H, m), 7.02-7.38 (16H, m), 7.39-7.49 (2H, m), 7.51-7.61 (1H, m), 7.99-8.08 (2H, m);

MASS (ES+): m/e 847.30 (M+1).

Preparation 457

Compound (457) was obtained in a manner similar to Preparation 17.

¹H-NMR (300 MHz, CDCl₃, δ): 1.08-2.26 (10H, m), 1.41 (6H, s), 1.42 (6H, s), 2.67-3.15 (1H, m), 2.94-3.15 (3H, m), 3.30-3.44 (1H, m), 3.59-3.74 (1H, m), 3.88-4.02 (1H, m), 4.21-4.40 (3H, m), 4.86-5.00 (1H, m), 5.08-5.25 (1H, m), 6.52 (1H, s), 7.02-7.10 (1H, m), 7.17-7.33 (10H, m), 7.34-7.47 (2H, m), 7.52-7.61 (1H, m), 8.03 (2H, d, J=7.7 Hz);

MASS (ES+): m/e 757.30 (M+1).

Preparation 458

Compound (458) was obtained in a manner similar to Preparation 18.

¹H-NMR (300 MHz, CDCl₃, δ): 1.11-2.18 (10H, m), 1.43 (3H, s), 2.68-2.96 (2H, m), 2.98-3.26 (2H, m), 3.62-3.80 (1H, m), 3.96-4.32 (3H, m), 4.32-4.63 (1H, m), 4.64-4.92 (1H, m), 6.94-7.31 (11H, m), 7.31-7.44 (2H, m), 7.44-7.55 (1H, m), 7.80-8.10 (2H, m), 8.18-8.79 (3H, m);

MASS (ES+): m/e 657.34 (M+1).

Preparation 459

Compound (459) was obtained in a manner similar to Preparation 76.

¹H-NMR (300 MHz, CDCl₃, δ): 1.30-1.91 (8H, m), 1.72 (3H, S), 1.96-2.17 (1H, m), 2.00-2.34 (1H, m), 2.90-3.00 (2H, m), 3.08-3.30 (3H, m), 3.71-3.83 (1H, m), 4.14-4.43 (1H, m), 4.29 (2H, t, J=6.3 Hz), 4.60-4.66 (1H, m), 5.08-5.20 (1H, m), 6.16 (1H, s), 7.09 (1H, d, J=9.9 Hz), 7.17-7.36 (10H, m), 7.36-7.50 (3H, m), 7.50-7.62 (1H, m), 8.03 (2H, d, J=7.3 Hz);

MASS (ES+): m/e 639.37 (M+1).

Preparation 460

Compound (460) was obtained in a manner similar to Preparation 77.

¹H-NMR (300 MHz, CDCl₃, δ): 1.19-1.85 (8H, m), 1.72 (3H, s), 2.00-2.15 (1H, m), 2.21-2.33 (1H, m), 2.93-3.02 (1H, m), 2.95 (1H, d, J=13.9 Hz), 3.12-3.31 (2H, m), 3.18 (1H, d, J=13.9 Hz), 3.62 (2H, t, J=6.3 Hz), 3.72-3.83 (1H, m), 4.11-4.24 (1H, m), 4.59-4.68 (1H, m), 5.08-5.21 (1H, m), 6.15 (1H, s), 7.05 (1H, d, J=10.3 Hz), 7.18-7.40 (10H, m), 7.37 (1H, d, J=10.3 Hz);

MASS (ES+): m/e 535.31 (M+1).

Preparation 461

Compound (461) was obtained in a manner similar to Preparation 78. The obtained compound was used in Example 249.

¹H-NMR (300 MHz, CDCl₃, δ): 1.40-1.86 (6H, m), 1.72 (3H, s), 1.98-2.17 (1H, m), 2.20-2.32 (1H, m), 2.36 (1H, t, J=6.6 Hz), 2.46 (1H, t, J=6.6 Hz), 2.88-3.01 (1H, m), 2.95 (1H, d, J=13.9 Hz), 3.06-3.30 (2H, m), 3.21 (1H, d, J=13.9 Hz), 3.70-3.84 (1H, m), 4.06-4.32 (1H, m), 4.59-4.70 (1H, m), 5.07-5.19 (1H, m), 6.11 (0.2H, s), 6.22 (0.5H, s), 6.39 (0.3H, s), 7.08 (1H, d, J=9.9 Hz), 7.18-7.41 (10H, m), 7.35 (1H, d, J=9.5 Hz), 9.73 (1H, s);

MASS (ES+): m/e 533.24 (M+1).

Preparation 462

Compound (462) was obtained in a manner similar to Preparation 13.

¹H-NMR (300 MHz, CDCl₃, δ): 1.48 (9H, brs), 1.55 (3H, brs), 3.31 (2H, brs), 5.04 (1H, brs), 7.10-7.18 (2H, m), 7.21-7.33 (3H, m);

MASS (ES+): m/e 280.12 (M+1).

Preparation 463

Compound (463) was obtained in a manner similar to Preparation 15.

¹H-NMR (300 MHz, CDCl₃, δ): 1.31 (3H, s), 1.38-1.67 (2H, m), 1.41 (2H, s), 1.49 (7H, s), 1.70-1.96 (2H, m), 2.55 (1H, dt, J=9.9, 7.3 Hz), 2.90 (1H, dd, J=12.8, 10.3 Hz), 3.06-3.23 (1H, m), 3.14 (1H, dd, J=12.8, 4.8 Hz), 3.32-3.65 (2H, m), 4.33-4.39 (1H, m), 4.67-4.79 (1H, m), 4.95 (1H, ddd, J=10.3, 8.4, 4.8 Hz), 5.09 (1H, d, J=12.5 Hz), 5.17 (1H, d, J=12.5 Hz), 6.95 (1H, d, J=8.4 Hz), 7.06-7.16 (2H, m), 7.18-7.41 (13H, m);

MASS (ES+): m/e 614.39 (M+1).

Preparation 464

Compound (464) was obtained in a manner similar to Preparation 16.

¹H-NMR (300 MHz, CDCl₃, δ): 1.30-2.01 (10H, m), 1.41 (9H, s), 1.51 (3H, s), 2.62-2.74 (1H, m), 2.87-3.19 (3H, m), 3.36-3.67 (2H, m), 4.00-4.16 (1H, m), 4.20-4.42 (3H, m), 4.85-5.00 (1H, m), 5.05-5.25 (3H, m), 6.76-7.08 (1H, m), 6.97-7.08 (2H, m), 7.09-7.35 (13H, m), 7.37-7.47 (2H, m), 7.49-7.59 (1H, m), 7.97-8.06 (2H, m);

MASS (ES+): m/e 847.31 (M+1).

Preparation 465

Compound (465) was obtained in a manner similar to Preparation 17.

¹H-NMR (300 MHz, CDCl₃, δ): 1.09-2.00 (10H, m), 1.41 (9H, s), 1.44 (3H, s), 2.58-2.70 (1H, m), 2.91-3.10 (2H, m), 3.17 (1H, d, J=13.9 Hz), 3.34 (1H, d, J=13.9 Hz), 3.52-3.66 (1H, m), 3.91-4.03 (1H, m), 4.22-4.37 (1H, m), 4.31 (2H, t, J=6.3 Hz), 4.83-4.94 (1H, m), 5.10-5.23 (1H, m), 6.67 (1H, s), 7.03-7.09 (1H, m), 7.16-7.34 (10H, m), 7.38-7.47 (2H, m), 7.52-7.59 (1H, m), 8.03 (2H, d, J=7.0 Hz);

MASS (ES+): m/e 757.33 (M+1).

Preparation 466

Compound (466) was obtained in a manner similar to Preparation 18.

¹H-NMR (300 MHz, CDCl₃, δ): 1.19-2.20 (11H, m), 1.41 (3H, s), 2.79-3.18 (3H, m), 3.30-3.44 (1H, m), 3.58-3.75 (1H, m), 4.02-4.42 (4H, m), 4.83-4.98 (1H, m), 7.05-7.31 (11H, m), 7.32-7.45 (2H, m), 7.45-7.54 (1H, m), 7.97 (2H, d, J=7.3 Hz), 8.04-8.08 (3H, m);

MASS (ES+): m/e 657.38 (Free).

Preparation 467

Compound (467) was obtained in a manner similar to Preparation 76.

¹H-NMR (300 MHz, CDCl₃, δ): 1.17 (3H, s), 1.29-2.39 (10H, m), 3.09 (1H, dd, J=13.7, 7.0 Hz), 3.23-3.38 (1H, m), 3.31 (1H, dd, J=13.7, 9.9 Hz), 3.39 (1H, d, J=13.9 Hz), 3.61 (1H, d, J=13.9 Hz), 3.81-3.91 (1H, m), 4.18-4.30 (1H, m), 4.34 (2H, t, J=6.4 Hz), 4.67-4.74 (1H, m), 5.22-5.33 (1H, m), 5.93 (1H, s), 6.97-7.05 (2H, m), 7.13-7.35 (9H, m), 7.39-7.48 (2H, m), 7.50-7.59 (1H, m), 7.86 (1H, d, J=10.3 Hz), 8.05 (2H, d, J=7.0 Hz);

MASS (ES+): m/e 639.35 (M+1).

Preparation 468

Compound (468) was obtained in a manner similar to Preparation 77.

¹H-NMR (300 MHz, CDCl₃, δ): 1.17 (3H, s), 1.32-1.49 (2H, m), 1.54-2.00 (6H, m), 2.11-2.25 (1H, m), 2.27-2.39 (1H, m), 3.08 (1H, dd, J=13.9, 7.0 Hz), 3.26-3.38 (1H, m), 3.29 (1H, dd, J=13.9, 8.8 Hz), 3.38 (1H, d, J=13.9 Hz), 3.63 (1H, d, J=13.9 Hz), 3.67 (2H, t, J=6.3 Hz), 3.80-3.91 (1H, m), 4.22 (1H, ddd, J=10.3, 8.1, 7.0 Hz), 4.67-4.75 (1H, m), 5.21-5.33 (1H, m), 5.99 (1H, s), 6.99-7.06 (2H, m), 7.15-7.35 (9H, m), 7.86 (1H, d, J=10.3 Hz);

MASS (ES+): m/e 535.30 (M+1).

Preparation 469

Compound (469) was obtained in a manner similar to Preparation 78. The obtained compound was used in Example 257.

¹H-NMR (300 MHz, CDCl₃, δ): 1.17 (3H, s), 1.55-1.99 (8H, m), 2.07-2.26 (1H, m), 2.27-2.37 (1H, m), 2.53 (2H, t, J=6.6 Hz), 3.09 (1H, dd, J=13.9, 7.0 Hz), 3.26-3.40 (1H, m), 3.29 (1H, dd, J=13.9, 9.1 Hz), 3.40 (1H, d, J=13.9 Hz), 3.61 (1H, d, J=13.9 Hz), 3.87-3.92 (1H, m), 4.18-4.30 (1H, m), 4.67-4.74 (1H, m), 5.22-5.33 (1H, m), 5.92 (1H, s), 6.98-7.06 (2H, m), 7.15-7.36 (9H, m), 7.81 (1H, d, J=9.9 Hz), 9.79 (1H, s);

MASS (ES+): m/e 533.24 (M+1).

Preparation 470

Compound (470) was obtained in a manner similar to Preparation 14.

¹H-NMR (300 MHz, CDCl₃, δ): 1.34 (2H, s), 1.42 (7H, s), 1.60-2.29 (4H, m), 2.85-3.01 (3H, m), 3.57-3.71 (1H, m), 4.36-4.47 (1H, m), 4.57-4.681 (1H, m), 5.11 (1H, d, J=J=12.3 Hz Hz), 5.22 (1H, d, J=J=12.3 Hz), 5.27-5.37 (1H, m), 6.37-7.14 (3H, m), 7.27-7.45 (5H, m);

MASS (ES+): m/e 489.29 (M+1).

Preparation 471

Compound (471) was obtained in a manner similar to Preparation 15.

¹H-NMR (300 MHz, CDCl₃, δ): 0.81 (3H, t, J=7.4 Hz), 1.21-1.49 (14H, m), 1.54-2.09 (5H, m), 2.90-3.04 (2H, m), 3.50-3.71 (2H, m), 4.42 (1H, dd, J=3.3, 8.4 Hz), 4.87-5.06 (1H, m), 5.10 (1H, d, J=12.5 Hz), 5.17 (1H, d, J=12.5 Hz), 6.66-7.13 (4H, m), 7.30-7.42 (5H, m);

MASS (ES+): m/e 588.36 (M+1).

Preparation 472

Compound (472) was obtained in a manner similar to Preparation 16.

¹H-NMR (300 MHz, CDCl₃, δ): 0.74 (3H, t, J=7.3 Hz), 1.34-1.46 (9H, m), 1.48-2.33 (16H, m), 2.75-3.09 (2H, m), 3.52-3.75 (2H, m), 3.92-4.13 (1H, m), 4.32 (2H, t, J=6.6 Hz), 4.39-4.45 (1H, m), 4.87-5.10 (1H, m), 5.10-5.21 (2H, m), 6.69-7.11 (4H, m), 7.29-7.37 (6H, m), 7.39-7.49 (2H, m), 7.52-7.63 (1H, m), 8.00-8.06 (2H, m);

MASS (ES+): m/e 821.23 (M+1).

Preparation 473

Compound (473) was obtained in a manner similar to Preparation 17.

¹H-NMR (300 MHz, CDCl₃, δ): 0.78 (3H, t, J=7.3 Hz), 1.42 (3H, s), 1.44 (9H, s), 1.62-1.99 (12H, m), 2.15-2.26 (1H, m), 2.81-3.10 (3H, m), 3.75-3.89 (1H, m), 4.00-4.17 (1H, m), 4.23-4.43 (3H, m), 4.85-4.95 (1H, m), 5.41-5.55 (1H, m), 6.78 (1H, brs), 6.91-7.15 (3H, m), 7.24-7.34 (1H, m), 7.40-7.51 (2H, m), 7.52-7.62 (1H, m), 8.00-8.08 (2H, m);

MASS (ES+): m/e 731.25 (M+1).

Preparation 474

Compound (474) was obtained in a manner similar to Preparation 18.

¹H-NMR (300 MHz, CDCl₃, δ): 0.63-0.78 (3H, m), 1.37 (3H, s); 1.53-2.16% (15H, m), 2.81-3.28 (3H, m), 3.71-3.86 (1H, m), 4.16-4.42 (4H, m), 4.86-5.01 (1H, m), 6.90-7.14 (3H, m), 7.36-7.46 (2H, m), 7.50-7.59 (1H, m), 7.66-7.83 (1H, m), 8.09-8.33 (3H, m);

MASS (ES−): m/e 665.32 (M−1).

Preparation 475

Compound (475) was obtained in a manner similar to Preparation 76.

¹H-NMR (300 MHz, CDCl₃, δ): 0.81 (3H, t, J=7.5 Hz), 1.28 (3H, s), 1.36-1.56 (2H, m), 1.63-2.00 (6H, m), 2.06-2.42 (4H, m), 2.93 (1H, dd, J=13.6, 6.6 Hz), 3.18 (1H, dd, J=13.6, 9.3 Hz), 3.30 (1H, dt, J=10.3, 7.3 Hz), 3.80-3.90 (1H, m), 4.20-4.30 (1H, m), 4.32 (2H, t, J=6.3 Hz), 4.66-4.72 (1H, m), 5.12 (1H, dt, J=9.5, 6.6 Hz), 5.86 (1H, s), 6.91-6.99 (1H, m), 7.01-7.12 (3H, m), 7.40-7.48 (2H, m), 7.53-7.63 (2H, m), 8.01-8.06 (2H, m);

MASS (ES+): m/e 613.28 (M+1).

Preparation 476

Compound (476) was obtained in a manner similar to Preparation 77.

¹H-NMR (300 MHz, CDCl₃, δ): 0.83 (3H, t, J=7.7 Hz), 1.22-1.73 (6H, m), 1.28 (3H, s), 1.74-1.94 (3H, m), 2.08-2.41 (4H, m), 2.92 (1H, dd, J=13.6, 6.6 Hz), 3.18 (1H, dd, J=13.6, 9.2 Hz), 3.30 (1H, dt, J=10.3, 7.3 Hz), 3.66 (2H, t, J=6.2 Hz), 3.85 (1H, ddd, J=10.3, 8.8, 5.1 Hz), 4.24 (1H, dt, J=10.3, 7.7 Hz), 4.66-4.72 (1H, m), 5.11 (1H, dt, J=9.2, 6.6 Hz), 5.94 (1H, s), 6.91-6.98 (1H, m), 7.00-7.11 (3H, m), 7.59 (1H, d, J=10.3 Hz);

MASS (ES+): m/e 509.54 (M+1).

Preparation 477

Compound (477) was obtained in a manner similar to Preparation 78. The obtained compound was used in Example 266.

¹H-NMR (300 MHz, CDCl₃, δ): 0.84 (3H, t, J=7.3 Hz), 1.29 (3H, s), 1.45-1.92 (6H, m), 2.07-2.41 (4H, m), 2.46-2.55 (2H, m), 2.92 (1H, dd, J=13.6, 6.6 Hz), 3.18 (1H, dd, J=13.6, 9.2 Hz), 3.30 (1H, dt, J=10.3, 7.0 Hz), 3.79-3.90 (1H, m), 4.24 (1H, dt, J=10.3, 7.0. Hz), 4.65-4.72 (1H, m), 5.12 (1H, ddd, J=10.2, 9.2, 6.6 Hz), 5.86 (1H, s), 6.91-6.98 (1H, m), 7.00-7.12 (3H, m), 7.53 (1H, d, J=10.3 Hz), 9.77 (1H, r, J=1.1 Hz);

MASS (ES+): m/e 507.29 (M+1).

Preparation 478

The Compound (343) (1.75 g) was dissolved into tetrahydrofuran (20 ml). To this solution was added (tert-butoxycarbonylmethylene)triphenylphosphoran (2.18 g) and stirred at ambient temperature overnight. The solvent was evaporated and the residue was purified by flash column chromatography (Silica gel 60N, Spherical, 120 g, eluting with ethyl acetate/hexane=1/1) to give the objective Compound (478) as a white foam.

¹H-NMR (300 MHz, CDCl₃, δ): 0.80 (3H, t, J=7.3 Hz), 1.27 (3H, s), 1.38-1.54 (2H, m), 1.53 (3×3H, s), 1.58-1.98 (6H, m), 2.06-2.38 (4H, m), 2.98 (1H, dd, J=13.7, 6.4 Hz), 3.24 (1H, dd, J=13.7, 9.5 Hz), 3.28 (1H, m), 3.85 (1H, m), 4.24 (1H, dt, J=10.3, 7.7 Hz), 4.32 (2H, t, J=6.5 Hz), 4.68 (1H, m), 5.18 (1H, m), 5.89 (1H, s), 6.33 (1H, d, J=16 Hz), 7.10 (1H, d, J=10.3 Hz), 7.24 (2×1H, d, J=8.2 Hz), 7.39-7.48 (4H, m), 7.50-7.62 (3H, m), 8.03 (2×1H, dd, J=7.5, 1.5 Hz);

MASS (ES+): m/e 703.54.

Preparation 479

Compound (479) was obtained in a manner similar to Example 3 mentioned below.

¹H-NMR (300 MHz, CDCl₃, δ): 0.81 (3H, t, J=7.3 Hz), 1.28 (3H, s), 1.34-1.54 (2H, m), 1.62-1.97 (6H, m), 2.09-2.38 (4H, m), 2.66 (2H, t, J=7.5 Hz), 2.91 (2H, t, J=7.5 Hz), 2.93 (1H, m), 3.17 (1H, dd, J=13.6, 9.5 Hz), 3.27 (1H, m), 3.84 (1H, m), 4.25 (1H, m), 4.32 (2H, t, J=6.5 Hz), 4.70 (1H, m), 5.16 (1H, m), 6.16 (1H, s), 7.11 (2×1H, d, J=8.2 Hz), 7.14 (2×1H, d, J=8.2 Hz), 7.27 (1H, d, J=10.3 Hz), 7.43 (2×1H, dd, J=7.5, 7.5 Hz), 7.56 (1H, dddd, J=7.5, 7.5, 1.5, 1.5 Hz), 7.65 (1H, d, J=10 Hz), 8.03 (2×1H, dd, J=7.5, 1.5 Hz);

MASS (ES+): m/e 705.49.

Preparation 480

The Compound (476) (1537 mg) was dissolved in dichloromethane (15 ml). To the mixture was added cold trifluoroacetic acid (5 ml) and stirred at ambient temperature for 30 min. The solvent was evaporated and the residue was azeotropically distillated with toluene. The residue was dissolved in ethyl acetate, washed with saturated brine (×2) and dried over sodium sulfate. The solvent was removed by evaporation to give the object Compound (480).

¹H-NMR (300 MHz, CDCl₃, δ): 0.81 (3H, t, J=7.3 Hz), 1.28 (3H, s), 1.34-1.54 (2H, m), 1.62-1.97 (6H, m), 2.09-2.38 (4H, m), 2.66 (2H, t, J=7.5 Hz), 2.91 (2H, t, J=7.5 Hz), 2.93 (1H, m), 3.17 (1H, dd, J=13.6, 9.5 Hz), 3.27 (1H, m), 3.84 (1H, m), 4.25 (1H, m), 4.32 (2H, t, J=6.5 Hz), 4.70 (1H, m), 5.16 (1H, m), 6.16 (1H, s), 7.11 (2×1H, d, J=8.2 Hz), 7.14 (2×1H, d, J=8.2 Hz), 7.27 (1H, d, J=10.3 Hz), 7.43 (2×1H, dd, J=7.5, 7.5 Hz), 7.56 (1H, dddd, J=7.5, 7.5, 1.5, 1.5 Hz), 7.65 (1H, d, J=10 Hz), 8.03 (2×1H, dd, J=7.5, 1.5 Hz);

MASS (ES−): m/e 649.56.

Preparation 481

Compound (481) was obtained in a manner similar to Preparation 301.

¹H-NMR (300 MHz, CDCl₃, δ): 0.82 (3H, t, J=7.3 Hz), 1.27 (3H, s), 1.38-2.00 (14H, m), 2.08-2.40 (4H, m), 2.58 (2H, m), 2.86-2.98 (3H, m), 3.21 (1H, dd, J=14, 9.5 Hz), 3.23-3.38 (3H, m), 3.55 (2H, m), 3.87 (1H, m), 4.24 (1H, m), 4.32 (2H, t, J=6.5 Hz), 4.68 (1H, m), 5.16 (1H, m), 5.87 (1H, s), 7.08-7.19 (5H, m), 7.44 (2×1H, dd, J=7.5, 7.5 Hz), 7.51-7.60 (2H, m), 8.03 (2×1H, dd, J=7.5, 2 Hz);

MASS (ES+): m/e 716.56.

Preparation 482

Compound (482) was obtained in a manner similar to Preparation 77.

¹H-NMR (300 MHz, CDCl₃, δ): 0.84 (3H, t, J=7.3 Hz), 1.24-1.93 (14H, m), 1.28 (3H, s), 2.08-2.40 (4H, m), 2.58 (2H, m), 2.86-2.97 (3H, m), 3.20 (1H, dd, J=14, 10 Hz), 3.22-3.37 (3H, m), 3.55 (2H, m), 3.65 (2H, t, J=6.5 Hz), 3.87 (1H, m), 4.23 (1H, dt, J=10.2, 7.7 Hz), 4.68 (1H, dd, J=8, 2 Hz), 5.16 (1H, m), 5.94 (1H, s), 7.08-7.18 (5H, m), 7.54 (1H, d, J=10.2 Hz);

MASS (ES+): m/e 612.62.

Preparation 483

Compound (483) was obtained in a manner similar to Preparation 78. The obtained compound was used in Example 269.

¹H-NMR (300 MHz, CDCl₃, δ): 0.84 (3H, t, J=7.3 Hz), 1.29 (3H, s), 1.42-1.90 (12H, m), 2.07-2.40 (4H, m), 2.50 (2H, m), 2.58 (2H, m), 2.86-2.98 (3H, m), 3.20 (1H, dd, J=14, 9.5 Hz), 3.22-3.38 (3H, m), 3.55 (1H, m), 3.87 (1H, m), 4.23 (1H, m), 4.68 (1H, dd, J=8, 2 Hz), 5.16 (1H, m), 5.91 (1H, s), 7.09-7.18 (5H, m), 7.49 (1H, d, J=10 Hz), 9.77 (1H, s);

MASS (ES+): m/e 610.57.

Preparation 484

Compound (484) was obtained in a manner similar to Preparation 303.

¹H-NMR (300 MHz, CDCl₃, δ): 0.80 (3H, t, J=7.3 Hz), 1.26 (3H, s), 1.36-1.98 (8H, m), 2.06-2.38 (4H, m), 2.63 (2H, t, J=7.4 Hz), 2.94 (1H, dd, J=13.5, 6.2 Hz), 3.01 (2H, t, J=7.4 Hz), 3.20 (1H, dd, J=13.5, 9.5 Hz), 3.26 (1H, m), 3.84 (1H, m), 4.24 (1H, m), 4.31 (2H, t, J=6.4 Hz), 4.66 (1H, m), 5.15 (1H, m), 5.86 (1H, s), 6.98-7.20 (7H, m), 7.30 (2×1H, dd, J=7.5, 7.5 Hz), 7.39-7.48 (4H, m), 7.51-7.60 (2H, m), 8.03 (2×1H, d, J=7.5 Hz);

MASS (ES+): m/e 724.40.

Preparation 485

Compound (485) was obtained in a manner similar to Preparation 77.

¹H-NMR (300 MHz, CDCl₃, δ): 0.82 (3H, t, J=7.3 Hz), 1.27 (3H, s), 1.30-1.93 (8H, m), 2.06-2.38 (4H, m), 2.63 (2H, t, J=7.5 Hz), 2.93 (1H, dd, J=13.7, 6.2 Hz), 3.01 (2H, t, J=7.5 Hz), 3.20 (1H, dd, J=13.7, 9.5 Hz), 3.26 (1H, m), 3.65 (2H, t, J=6.3 Hz), 3.84 (1H, m), 4.22 (1H, dt, J=10, 7.5 Hz), 4.66 (1H, dd, J=8, 2 Hz), 5.15 (1H, ddd, J=10.3, 9.5, 6.2 Hz), 5.93 (1H, s), 7.00-7.20 (7H, m), 7.30 (2×1H, dd, J=7.5, 7.5 Hz), 7.43 (2×1H, d, J=7.5 Hz), 7.54 (1H, d, J=10.3 Hz);

MASS (ES+): m/e 620.50.

Preparation 486

Compound (486) was obtained in a manner similar to Preparation 78. The obtained compound was used in Example 272.

¹H-NMR (300 MHz, CDCl₃, δ): 0.82 (3H, t, J=7.3 Hz), 1.28 (3H, s), 1.52-1.92 (6H, m), 2.08-2.38 (4H, m), 2.50 (2H, m), 2.63 (2H, t, J=7.5 Hz), 2.93 (1H, dd, J=13.5, 6.2 Hz), 3.01 (2H, t, J=7.5 Hz), 3.20 (1H, dd, J=13.5, 9.5 Hz), 3.25 (1H, m), 3.84 (1H, m), 4.23 (1H, m), 4.66 (1H, dd, J=8, 2 Hz), 5.15 (1H, ddd, J=10.3, 9.5, 6.2 Hz), 5.93 (1H, s), 7.02-7.21 (7H, m), 7.30 (2×1H, dd, J=8, 8 Hz), 7.43 (2×1H, d, J=8 Hz), 7.49 (1H, d, J=10.3 Hz), 9.77 (1H, s);

MASS (ES+): m/e 618.56.

Preparation 487

Compound (487) was obtained in a manner similar to Preparation 13.

¹H-NMR (300 MHz, CDCl₃, δ): 1.22 (1H, m), 1.37 (9×½H, s), 1.45 (9×½H, s), 1.54-1.74 (4H, m), 2.23 (1H, m), 2.80-3.03 (1H, m), 3.82-4.09 (1H, m), 4.75 (½H, m), 4.95 (½H, m), 5.07-5.25 (2H, m), 7.24-7.40 (5H, m);

MASS (ES+): m/e 320.48.

Preparation 488

Compound (488) was obtained in a manner similar to Preparation 13.

¹H-NMR (300 MHz, CDCl₃; δ): 1.48-2.34 (6H, m), 3.08 (1H, m), 3.61 (1H, m), 3.99 (1H, dd, J=9, 4 Hz), 5.21 (1H, d, J=12 Hz), 5.26 (1H, d, J=12 Hz), 7.29-7.41 (5H, m);

MASS (ES+): m/e 220.37.

Preparation 489

Compound (489) was obtained in a manner similar to Preparation 14.

¹H-NMR (300 MHz, CDCl₃, δ): 0.59 (1H, m), 1.15 (1H, m), 1.30-1.76 (3H, m), 1.42 (3×3H, s), 2.20 (1H, m), 2.90-3.18 (3H, m), 3.57 (1H, m), 4.86-5.00 (1H, m), 5.08-5.24 (3H, m), 5.29 (1H, brd, J=4.5 Hz), 5.44 (1H, d, J=9 Hz), 7.16-7.44 (10H, m);

MASS (ES+): m/e 467.54.

Preparation 490

Compound (490) was obtained in a manner similar to Preparation 15.

¹H-NMR (300 MHz, CDCl₃, δ): 0.25 (1H, m), 1.02 (1H, m), 1.18-1.72 (3H, m), 2.14 (1H, m), 3.00-3.24 (2H, m), 3.42 (1H, m), 3.60 (1H, m), 4.88-5.22 (4H, m), 7.17-7.44 (10H, m), 8.60 (2H, br);

MASS (ES+): m/e 367.49.

Preparation 491

Compound (491) was obtained in a manner similar to Preparation 15.

¹H-NMR (300 MHz, CDCl₃, δ): 0.58 (1H, m), 1.13 (1H, m), 1.30-1.76 (3H, m), 1.39 (3×3H, s), 2.19 (1H, m), 2.88-3.16 (5H, m), 3.51 (1H, m), 4.35 (1H, m), 4.94 (1H, m), 5.09-5.28 (4H, m), 6.77 (2×1H, d, J=8 Hz), 7.08-7.38 (15H, m);

MASS (ES+): m/e 614.

Preparation 492

Compound (492) was obtained in a manner similar to Preparation 16.

¹H-NMR (300 MHz, CDCl₃, δ): 0.43 (1H, m), 1.10 (1H, m), 1.20-1.50 (3H, m), 2.13 (1H, m), 2.89-3.16 (3H, m), 3.20-3.42 (2H, m), 3.52 (1H, m), 4.45 (1H, m), 5.04-5.22 (4H, m), 7.08-7.40 (15H, m), 7.73 (1H, d, J=7.7 Hz), 8.58 (2H, br);

MASS (ES+): m/e 514.

Preparation 493

Compound (493) was obtained in a manner similar to Preparation 16.

¹H-NMR (300 MHz, CDCl₃, δ): 0.60 (1H, m), 1.14 (1H, m), 1.32-1.90 (9H, m), 1.44 (3×3H, s), 2.20 (1H, m), 2.89-3.06 (4H, m), 3.11 (1H, m), 3.52 (1H, m), 4.07 (1H, m), 4.28 (2H, t, J=6.5 Hz), 4.63 (1H, m), 4.93 (1H, m), 5.06-5.21 (3H, m), 5.26 (1H, brd, J=4.5 Hz), 6.61 (1H, d, J=7.7 Hz), 6.69 (1H, d, J=8 Hz), 7.08-7.38 (15H, m), 7.43 (2×1H, dd, J=7.5, 7.5 Hz), 7.55 (1H, m), 8.02 (2×1H, dd, J=7.5, 1.5 Hz);

MASS (ES+): m/e 847.58.

Preparation 494

Compound (494) was obtained in a manner similar to Preparation 17.

¹H-NMR (300 MHz, CDCl₃, δ): 0.67 (1H, m), 1.12-1.84 (10H, m), 1.42 (3×3H, s), 2.20 (1H, m), 2.87-3.15 (5H, m), 3.56 (1H, m), 4.07 (1H, m), 4.26 (2H, t, J=6.8 Hz), 4.74 (1H, m), 5.00-5.20 (3H, m), 6.85 (2×1H, d, J=8.5 Hz), 7.05-7.32 (10H, m), 7.37-7.48 (3H, m), 7.55 (1H, m), 8.02 (2×1H, dd, J=7.5, 1 Hz);

MASS (ES+): m/e 757.

Preparation 495

Compound (495) was obtained in a manner similar to Preparation 18.

¹H-NMR (300 MHz, CDCl₃, δ): 0.76 (1H, m), 0.98-2.00 (10H, m), 2.14 (1H, m), 2.88-3.10 (5H, m), 3.55 (1H, m), 3.96 (1H, m), 4.14 (2H, m), 4.52 (1H, m), 5.00-5.15 (2H, m), 7.08-7.32 (10H, m), 7.39 (2×1H, dd, J=7.5, 7.5 Hz), 7.52 (1H, dd, J=7.5, 7.5 Hz), 7.84 (1H, br), 7.98 (2×1H, d, J=7.5 Hz), 8.24 (2H, br), 8.61 (1H, br);

MASS (ES+): m/e 657.

Preparation 496

Compound (496) was obtained in a manner similar to Preparation 76.

¹H-NMR (300 MHz, CDCl₃, δ): 1.20-2.16 (12H, m), 3.01 (1H, m), 3.08 (1H, dd, J=14, 7 Hz), 3.21 (1H, dd, J=13, 5.5 Hz), 3.26 (1H, dd, J=14, 8 Hz), 3.64 (1H, dd, J=13, 10.5 Hz), 3.72 (1H, ddd, J=10.5, 6, 5.5 Hz), 3.95 (1H, m), 4.20 (1H, m), 4.29 (2H, m), 5.01 (1H, m), 5.36 (1H, m), 6.41 (1H, d, J=6 Hz), 6.48 (1H, d, J=10.5 Hz), 7.05-7.12 (2H, m), 7.14-7.34 (8H, m), 7.39-7.49 (3H, m), 7.56 (1H, m), 8.04 (2H, m);

MASS (ES+): m/e 639.33.

Preparation 497

Compound (497) was obtained in a manner similar to Preparation 77.

¹H-NMR (300 MHz, CDCl₃, δ): 1.14-1.88 (10H, m), 1.91-2.15 (2H, m), 2.98 (1H, m), 3.07 (1H, dd, J=14, 7.5 Hz), 3.21 (1H, dd, J=14, 7 Hz), 3.24 (1H, dd, J=14, 8 Hz), 3.55-3.67 (3H, m), 3.76 (1H, m), 3.94 (1H, m), 4.21 (1H, m), 5.04 (1H, m), 5.35 (1H, ddd, J=10, 7.5, 7 Hz), 6.56 (1H, d, J=10.5 Hz), 6.98 (1H, d, J=6 Hz), 7.07-7.14 (8H, m), 7.15-7.34 (8H, m), 7.50 (1H, d, J=10 Hz);

MASS (ES+): m/e 535.36.

Preparation 498

Compound (498) was obtained in a manner similar to Preparation 78. The obtained compound was used in Example 275.

¹H-NMR (300 MHz, CDCl₃, δ): 1.24 (1H, m), 1.42-1.88 (7H, m), 1.91-2.15 (2H, m), 2.45 (2H, m), 3.01 (1H, m), 3.07 (1H, dd, J=14, 7.5 Hz), 3.21 (1H, dd, J=13.5, 6 Hz), 3.25 (1H, dd, J=14, 8.5 Hz), 3.63 (1H, dd, J=13.5, 10.5 Hz), 3.76 (1H, ddd, J=10.5, 6, 5.5 Hz), 3.95 (1H, m), 4.20 (1H, m), 5.02 (1H, m), 5.36 (1H, ddd, J=10, 8.5, 7.5 Hz), 6.49 (1H, d, J=10 Hz), 6.53 (1H, d, J=5.5 Hz), 7.06-7.12 (2H, m), 7.16-7.34 (8H, m), 7.39 (1H, d, J=10 Hz), 9.73 (1H, s);

MASS (ES−): m/e 531.35.

Preparation 499

Compound (499) was obtained in a manner similar to Preparation 14.

¹H-NMR (300 MHz, CDCl₃, δ): 1.34 (1H, m), 1.63-1.84 (2H, m), 2.16-2.46 (3H, m), 3.16 (1H, m), 3.66 (1H, m), 4.32 (1H, m), 4.68 (1H, m), 5.05 (1H, d, J=12 Hz), 5.13 (1H, d, J=12 Hz), 7.16-7.38 (10H, m), 8.70 (2H, br);

MASS (ES+): m/e 353.

Preparation 500

Compound (500) was obtained in a manner similar to Preparation 15.

¹H-NMR (300 MHz, CDCl₃, δ): 1.40 (3×3H, s), 1.51 (1H, m), 1.72-1.98 (3H, m), 2.62 (1H, m), 2.85-3.13 (4H, m), 3.44 (1H, m), 4.31-4.42 (2H, m), 4.84-4.99 (2H, m), 5.12 (1H, d, J=12.5 Hz), 5.16 (1H, d, J=12.5 Hz), 6.71 (1H, d, J=8 Hz), 7.06-7.40 (15H, m);

MASS (ES+): m/e 622.37 (M+Na).

Preparation 501

Compound (501) was obtained in a manner similar to Preparation 16.

¹H-NMR (300 MHz, CDCl₃, δ): 1.42 (1H, m), 1.65-2.18 (3H, m), 2.54 (1H, m), 2.89-3.60 (5H, m), 4.27 (1H, m), 4.50 (1H, m), 4.79 (1H, m), 5.06-5.20 (2H, m), 6.85 (1H, m), 7.06-7.40 (14H, m), 8.01 (1H, brd, J=7 Hz), 8.51 (2H, br);

MASS (ES+): m/e 500.27.

Preparation 502

Compound (502) was obtained in a manner similar to Preparation 16.

¹H-NMR (300 MHz, CDCl₃, δ): 1.32-2.25 (10H, m), 1.44 (3×3H, s), 2.62 (1H, m), 2.84-3.11 (4H, m), 3.45 (1H, m), 4.07 (1H, m), 4.29 (2H, t, J=6.5 Hz), 4.36 (1H, m), 4.62 (1H, m), 4.79-5.00 (2H, m), 5.13 (1H, d, J=12 Hz), 5.17 (1H, d, J=12 Hz), 6.56-6.66 (2H, m), 7.10-7.36 (15H, m), 7.43 (2×1H, dd, J=7.5, 7.5 Hz), 7.55 (1H, m), 8.02 (2×1H, dd, J=7.5, 1.5 Hz);

MASS (ES+): m/e 855.85 (M+Na).

Preparation 503

Compound (503) was obtained in a manner similar to Preparation 17.

¹H-NMR (300 MHz, CDCl₃, δ): 1.33-2.12 (10H, m), 1.42 (3×3H, s), 2.76 (1H, m), 2.87 (1H, dd, J=14, 5 Hz), 3.02-3.22 (3H, m), 3.60 (1H, m), 4.11 (1H, m), 4.22 (1H, m), 4.28 (2H, t, J=6.5 Hz), 4.85 (1H, m), 4.94 (1H, d, J=8.5 Hz), 5.12 (1H, m), 6.91 (1H, d, J=7.7 Hz), 6.99 (2×1H, d, J=7 Hz), 7.08-7.32 (8H, m), 7.43 (2×1H, dd, J=7.5, 7.5 Hz), 7.55 (1H, m), 8.03 (2×1H, d, J=7.5 Hz), 8.31 (1H, brd, J=8.5 Hz);

MASS (ES−): m/e 741.96.

Preparation 504

Compound (504) was obtained in a manner similar to Preparation 18.

¹H-NMR (300 MHz, CDCl₃, δ): 1.02-2.05 (10H, m), 2.72-3.18 (5H, m), 3.53 (1H, m), 3.93-4.30 (4H, m), 4.62 (1H, m), 4.84 (1H, m), 7.04-7.32 (11H, m), 7.39 (2×1H, dd, J=7.5, 7.5 Hz), 7.52 (1H, dd, J=7.5, 7.5 Hz), 7.98 (2×1H, d, J=7.5 Hz), 8.30 (2H, br), 8.54 (1H, br);

MASS (ES+): m/e 643.78.

Preparation 505

Compound (505) was obtained in a manner similar to Preparation 76.

¹H-NMR (300 MHz, CDCl₃, δ): 1.31-1.45 (2H, m), 1.60-1.98 (6H, m), 2.08-2.36 (2H, m), 3.02 (1H, dd, J=14, 6 Hz), 3.16-3.36 (3H, m), 3.60-3.79 (2H, m), 3.86 (1H, m), 4.18 (1H, m), 4.29 (1H, t, J=6 Hz), 4.67 (1H, m), 5.16 (1H, m), 6.38 (1H, d, J=5 Hz), 7.08-7.34 (11H, m), 7.43 (2×1H, dd, J=7.5, 7.5 Hz), 7.48-7.60 (2H, m), 8.03 (2×1H, d, J=7.5 Hz);

MASS (ES+): m/e 625.54.

Preparation 506

Compound (506) was obtained in a manner similar to Preparation 77.

¹H-NMR (300 MHz, CDCl₃, δ): 1.22-1.39 (2H, m), 1.46-1.94 (6H, m), 2.07-2.37 (2H, m), 3.02 (1H, dd, J=13.5, 6 Hz), 3.22 (1H, m), 3.27 (1H, dd, J=13.5, 9 Hz), 3.31 (1H, dd, J=13.5, 6 Hz), 3.63 (2H, t, J=6.5 Hz), 3.68 (1H, dd, J=13.5, 10.5 Hz), 3.74 (1H, ddd, J=10.5, 6, 6 Hz), 3.85 (1H, m), 4.18 (1H, m), 4.68 (1H, m), 5.16 (1H, ddd, J=10, 9, 6 Hz), 6.52 (1H, d, J=6 Hz), 7.10-7.34 (11H, m), 7.53 (1H, d, J=10 Hz);

MASS (ES+): m/e 519.90.

Preparation 507

Compound (50.7) was obtained in a manner similar to Preparation 78. The obtained compound was used in Example 281.

¹H-NMR (300 MHz, CDCl₃, δ): 1.42-1.96 (6H, m), 2.06-2.37 (2H, m), 2.45 (2H, m), 3.02 (1H, dd, J=14, 6 Hz), 3.22 (1H, m), 3.27 (1H, dd, J=14, 10 Hz), 3.32 (1H, dd, J=13, 6 Hz), 3.67 (1H, dd, J=13, 10 Hz), 3.75 (1H, ddd, J=10, 6, 5 Hz), 3.86 (1H, m), 4.18 (1H, m), 4.68 (1H, m), 5.16 (1H, ddd, J=10, 10, 6 Hz), 6.45 (1H, d, J=5 Hz), 7.10-7.40 (11H, m), 7.49 (1H, d, J=10 Hz), 9.74 (1H, s);

MASS (ES+): m/e 519.94.

Preparation 508

Compound (508) was obtained in a manner similar to Preparation 13.

¹H-NMR (300 MHz, CDCl₃, δ): 1.24 (3H, t, J=7 Hz), 1.40 (3H, t, J=7 Hz), 1.42 (3×3H, s), 3.02 (2H, m), 4.00 (2H, q, J=7 Hz), 4.16 (2H, q, J=7 Hz), 4.51 (1H, m), 4.96 (1H, brd, J=7 Hz), 6.81 (2×1H, d, J=8.7 Hz), 7.03 (2×1H, d, J=8.7 Hz);

MASS (ES+): m/e 338.47.

Preparation 509

Compound (509) was obtained in a manner similar to Preparation 14.

¹H-NMR (300 MHz, CDCl₃, δ): 1.40 (3H, t, J=7 Hz), 1.42 (3×3H, s), 3.03 (1H, dd, J=14, 6 Hz), 3.12 (1H, dd, J=14, 5.5 Hz), 4.01 (2H, q, J=7 Hz), 4.55 (1H, m), 4.92 (1H, brd, J=7.5 Hz), 6.83 (2×1H, d, J=8.5 Hz), 7.08 (2×1H, d, J=8.5 Hz);

MASS (ES−): m/e 308.50.

Preparation 510

Compound (510) was obtained in a manner similar to Preparation 15.

¹H-NMR (300 MHz, CDCl₃, δ): 0.58 (1H, m), 1.04-1.76 (4H, m), 1.38 (3H, t, J=7 Hz), 1.40 (3×3H, s), 2.19 (1H, m), 2.84-3.14 (5H, m), 3.51 (1H, m), 3.98 (2H, q, J=7 Hz), 4.31 (1H, m), 4.92 (1H, m), 5.08-5.23 (3H, m), 5.25 (1H, d, J=4 Hz), 6.75-6.85 (3H, m), 6.96-7.11 (3H, m), 7.13-7.41 (9H, m);

MASS (ES+): m/e 658.

Preparation 511

Compound (511) was obtained in a manner similar to Preparation 16.

¹H-NMR (300 MHz, CDCl₃, δ): 0.44 (1H, m), 1.00-2.19 (5H, m), 1.30 (3H, t, J=7 Hz), 2.88-3.58 (6H, m), 3.86 (2H, q, J=7 Hz), 4.41 (1H, m), 4.86-5.22 (4H, m), 6.65 (⅓H, d, J=8.5 Hz), 6.74 ( 5/3H, d, J=8.5 Hz), 6.89 (⅓H, d, J=8.5 Hz), 7.10-7.36 ( 35/3H, m), 7.82 (⅚H, d, J=7.5 Hz), 8.26 (⅙H, d, J=7.5 Hz), 8.52 (2H, br);

MASS (ES+): m/e 558.

Preparation 512

Compound (512) was obtained in a manner similar to Preparation 16.

¹H-NMR (300 MHz, CDCl₃, δ): 0.60 (1H, m), 1.14 (1H, m), 1.30-1.90 (9H, m), 1.37 (3H, t, J=7 Hz), 1.43 (3×3H, s), 2.20 (1H, m), 2.87-3.04 (4H, m), 3.10 (1H, m), 3.52 (1H, m), 3.96 (2H, q, J=7 Hz), 4.07 (1H, m), 4.29 (2H, t, J=6.5 Hz), 4.59 (1H, m), 4.94 (1H, m), 5.07-5.22 (3H, m), 5.26 (1H, brd, J=5 Hz), 6.58 (1H, d, J=8 Hz), 6.70 (1H, d, J=7.5 Hz), 6.79 (2×1H, d, J=8.5 Hz), 7.04 (2×1H, d, J=8.5 Hz), 7.09-7.38 (10H, m), 7.42 (2×1H, dd, J=7.5, 7.5 Hz), 7.54 (1H, m), 8.02 (2×1H, dd, J=7.5, 1 Hz);

MASS (ES+): m/e 891.

Preparation 513

Compound (513) was obtained in a manner similar to Preparation 17.

¹H-NMR (300 MHz, CDCl₃, δ): 0.67 (1H, m), 1.10-1.88 (10H, m), 1.36 (3H, t, J=7 Hz), 1.42 (3×3H, s), 2.20 (1H, m), 2.88-3.13 (5H, m), 3.52 (1H, m), 3.93 (2H, q, J=7 Hz), 4.06 (1H, m), 4.27 (2H, t, J=6.5 Hz), 4.68 (1H, m), 4.98-5.18 (3H, m), 6.69-6.81 (3H, m), 7.00 (2×1H, d, J=8.5 Hz), 7.10-7.34 (6H, m), 7.43 (2×1H, dd, J=7.5, 7.5 Hz), 7.55 (1H, m), 8.02 (2×1H, dd, J=7.5, 1.5 Hz);

MASS (ES+): m/e 800.

Preparation 514

Compound (514) was obtained in a manner similar to Preparation 18.

¹H-NMR (300 MHz, CDCl₃, δ): 0.76 (1H, m), 1.02-2.02 (10H, m), 1.27 (3H, t, J=7 Hz), 2.13 (1H, m), 2.85-3.12 (5H, m), 3.54 (1H, m), 3.83 (2H, br-q, J=7 Hz), 3.98 (1H, br), 4.15 (2H, br), 4.46 (1H, br), 4.99-5.15 (2H, m), 6.70 (2×1H, d, J=8 Hz), 7.06 (2×1H, d, J=8 Hz), 7.14-7.32 (5H, m), 7.38 (2×1H, dd, J=7.5, 7.5 Hz), 7.51 (1H, dd, J=7.5, 7.5 Hz), 7.81 (1H, br), 7.98 (2×1H, d, J=7.5 Hz), 8.26 (2H, br), 8.56 (1H, br);

MASS (ES+): m/e 701.

Preparation 515

Compound (515) was obtained in a manner similar to Preparation 76.

¹H-NMR (300 MHz, CDCl₃, δ): 1.14-2.16 (12H, m), 1.37 (3H, t, J=7 Hz), 3.01 (1H, m), 3.08 (1H, dd, J=14, 7 Hz), 3.15 (1H, dd, J=13.5, 6 Hz), 3.25 (1H, dd, J=14, 8 Hz), 3.55 (1H, dd, J=13.5, 10.5 Hz), 3.67 (1H, ddd, J=8, 6, 6 Hz), 3.94 (1H, m), 3.94 (2H, q, J=7 Hz), 4.21 (1H, m), 4.30 (2H, m), 5.02 (1H, m), 5.36 (1H, m), 6.44 (1H, d, J=6 Hz), 6.48 (1H, d, J=10 Hz), 6.73 (2×1H, d, J=8.5 Hz), 6.98 (2×1H, d, J=8.5 Hz), 7.18-7.34 (5H, m), 7.37-7.48 (3H, m), 7.55 (1H, m), 8.03 (2×1H, dd, J=8, 1.5 Hz);

MASS (ES+): m/e 683.43.

Preparation 516

Compound (516) was obtained in a manner similar to Preparation 77.

¹H-NMR (300 MHz, CDCl₃, δ): 1.14-1.85 (10H, m), 1.39 (3H, t, J=7 Hz), 1.96 (1H, m), 2.07 (1H, m), 2.90-3.29 (4H, m), 3.47-3.75 (4H, m), 3.94 (1H, m), 3.98 (1H, q, J=7 Hz), 4.20 (1H, m), 5.03 (1H, m), 5.35 (1H, m), 6.53 (1H, d, J=10 Hz), 6.76 (2×1H, d, J=8.5 Hz), 6.79 (2×1H, d, J=6.5 Hz), 6.99 (2×1H, d, J=8.5 Hz), 7.18-7.34 (5H, m), 7.45 (1H, d, J=10.5 Hz);

MASS (ES+): m/e 579.38.

Preparation 517

Compound (517) was obtained in a manner similar to Preparation 78. The obtained compound was used in Example 287.

¹H-NMR (300 MHz, CDCl₃, δ): 1.14-1.86 (8H, m), 1.39 (3H, t, J=7 Hz), 1.91-2.16 (2H, m), 2.46 (2H, m), 2.93-3.30 (4H, m), 3.54 (1H, d, J=14, 11 Hz), 3.71 (1H, m), 3.94 (1H, m), 3.98 (1H, q, J=7 Hz), 4.20 (1H, m), 5.02 (1H, m), 5.35 (1H, m), 6.49 (1H, d, J=10 Hz), 6.50 (1H, d, J=5.5 Hz), 6.76 (2×1H, d, J=8.5 Hz), 6.99 (2×1H, d, J=8.5 Hz), 7.15-7.35 (5H, m), 7.36 (1H, d, J=10 Hz), 9.74 (1H, s);

MASS (ES+): m/e 577.33.

Preparation 518

Compound (518) was obtained in a manner similar to Preparation 14.

¹H-NMR (300 MHz, CDCl₃, δ): 0.70 (1H, m), 1.17 (1H, m), 1.30-1.74 (3H, m), 1.39 (3H, t, J=7 Hz), 1.42 (3×3H, s), 2.21 (1H, m), 2.83-2.97 (2H, m), 3.13 (1H, m), 3.59 (1H, m), 3.99 (2H, q, J=7 Hz), 4.87 (1H, m), 5.08-5.23 (2H, m), 5.29 (1H, m), 5.42 (1H, d, J=8.5 Hz), 6.74 (0.2H, d, J=8.5 Hz), 6.80 (1.8H, d, J=8.5 Hz), 6.96 (0.2H, d, J=8.5 Hz), 7.09 (1.8H, d, J=8.5 Hz), 7.24-7.41 (5H, m);

MASS (ES+): m/e 511.29.

Preparation 519

Compound (519) was obtained in a manner similar to Preparation 15.

¹H-NMR (300 MHz, CDCl₃, δ): 0.49 (1H, m), 1.07 (1H, m), 1.22-1.74 (3H, m), 1.34 (3× 1/7H, t), 1.36 (3× 6/7H, t, J=7 Hz), 2.13 (1H, m), 3.00-3.31 (2H, m), 3.41-3.54 (2H, m), 3.89 (2× 1/7H, q, J=7 Hz), 3.95 (2× 6/7H, q, J=7 Hz), 4.84-5.22 (4H, m), 6.73 (2× 1/7H, d, J=8.5 Hz), 6.79 (2× 6/7H, d, J=8.5 Hz), 7.21 (2×1H, d, J=8.5 Hz), 7.25-7.40 (5H, m), 8.29 (2× 1/7H, br), 8.57 (2× 6/7H, br);

MASS (ES+): m/e 411.20.

Preparation 520

Compound (520) was obtained in a manner similar to Preparation 15.

¹H-NMR (300 MHz, CDCl₃, δ): 0.71 (1H, m), 1.06-1.70 (19H, m), 2.21 (1H, m), 2.82-3.16 (5H, m), 3.54 (1H, m), 3.91-4.04 (4H, m), 4.30 (1H, m), 4.92 (1H, m), 5.08-5.18 (3H, m), 5.26 (1H, m), 6.68-6.90 (5H, m), 7.00-7.12 (4H, m), 7.24-7.40 (5H, m);

MASS (ES+): m/e 702.35.

Preparation 521

Compound (521) was obtained in a manner similar to Preparation 16.

¹H-NMR (300 MHz, CDCl₃, δ): 0.58 (1H, m), 1.02-1.60 (10H, m), 2.15 (1H, m), 2.83-3.32 (5H, m), 3.56 (1H, m), 3.80-4.02 (4H, m), 4.38 (1H, m), 5.02-5.22 (4H, m), 6.63-6.93 (4H, m), 7.07 (2×1H, d, J=8.5 Hz), 7.16 (2×1H, d, J=8.5 Hz), 7.24-7.40 (5H, m), 7.71 (1×⅚H, brd, J=7 Hz), 8.19 (1×⅙H, brd, J=7 Hz), 8.50 (2H, br);

MASS (ES+): m/e 602.28.

Preparation 522

Compound (522) was obtained in a manner similar to Preparation 16.

¹H-NMR (300 MHz, CDCl₃, δ): 0.71 (1H, m), 1.06-1.90 (10H, m), 1.37 (3H, t, J=7 Hz), 1.38 (3H, t, J=7 Hz), 1.43 (3×3H, s), 2.21 (1H, m), 2.82-3.03 (4H, m), 3.12 (1H, m), 3.54 (1H, m), 3.95 (2H, q, J=7 Hz), 3.97 (2H, q, J=7 Hz), 4.07 (1H, m), 4.28 (2H, t, J=6.5 Hz), 4.59 (1H, m), 4.92 (1H, m), 5.05-5.21 (3H, m), 5.27 (1H, brd, J=4 Hz), 6.56 (1H, d, J=6.5 Hz), 6.61-6.88 (5H, m), 6.96-7.09 (4H, m), 7.24-7.38 (5H, m), 7.42 (2×1H, dd, J=7.5, 7.5 Hz), 7.54 (1H, dd, J=7.5, 7.5 Hz), 8.02 (2×1H, d, J=7.5 Hz);

MASS (ES−): m/e 968.89 (M+Cl).

Preparation 523

Compound (523) was obtained in a manner similar to Preparation 17.

¹H-NMR (300 MHz, CDCl₃, δ): 0.78 (1H, m), 1.18-1.88 (10H, m), 1.35 (3H, t, J=7 Hz), 1.38 (3H, t, J=7 Hz), 1.41 (3×3H, s), 2.21 (1H, m), 2.82-3.01 (4H, m), 3.09 (1H, m), 3.54 (1H, m), 3.93 (2H, q, J=7 Hz), 3.98 (2H, q, J=7 Hz), 4.16 (1H, m), 4.27 (2H, t, J=6.5 Hz), 4.68 (1H, m), 5.00-5.20 (3H, m), 6.67-6.83 (5H, m), 7.00 (2×1H, d, J=8.5 Hz), 7.06 (2×1H, d, J=8.5 Hz), 7.29 (1H, d, J=7.5 Hz), 7.43 (2×1H, dd, J=7.5, 7.5 Hz), 7.55 (1H, dd, J=7.5, 7.5 Hz), 8.02 (2×1H, d, J=7.5 Hz);

MASS (ES+): m/e 845.27.

Preparation 524

Compound (524) was obtained in a manner similar to Preparation 18.

¹H-NMR (300 MHz, CDCl₃, b): 0.75-2.01 (11H, m), 1.28 (3H, t, J=7 Hz), 1.35 (3H, t, J=7 Hz), 2.14 (1H, m), 2.80-3.12 (5H, m), 3.55 (1H, m), 3.74-4.02 (5H, m), 4.15 (2H, br), 4.46 (1H, m), 4.97-5.12 (2H, m), 6.71 (2×1H, brd, J=8 Hz), 6.77 (2×1H, brd, J=8 Hz), 7.00-7.20 (4H, m), 7.38 (2×1H, dd, J=7.5, 7.5 Hz), 7.51 (1H, dd, J=7.5, 7.5 Hz), 7.98 (2×1H, d, J=7.5 Hz), 8.26 (2H, br);

MASS (ES+): m/e 745.28.

Preparation 525

Compound (525) was obtained in a manner similar to Preparation 76.

¹H-NMR (300 MHz, CDCl₃, δ): 1.14-2.17 (12H, m), 1.37 (3H, t, J=7 Hz), 1.40 (3H, t, J=7 Hz), 2.92-3.06 (2H, m), 3.11-3.24 (2H, m), 3.54 (1H, dd, J=13.5, 10.5 Hz), 3.68 (1H, m), 3.87-4.05 (1H, m), 3.94 (2H, q, J=7 Hz), 4.00 (2H, q, J=7 Hz), 4.21 (1H, m), 4.29 (2H, t, J=6.5 Hz), 5.02 (1H, m), 5.30 (1H, m), 6.50 (1H, d, J=10 Hz), 6.53 (1H, d, J=5.5 Hz), 6.73 (2×1H, d, J=8.8 Hz), 6.82 (2×1H, d, J=8.8 Hz), 6.98 (2×1H, d, J=8.8 Hz), 7.16 (2×1H, d, J=8.8 Hz), 7.41 (1H, d, J=10.5 Hz), 7.44 (2×1H, dd, J=7.5, 7.5 Hz), 7.56 (1H, m), 8.03 (2×1H, dd, J=7.5, 1 Hz);

MASS (ES+): m/e 727.19.

Preparation 526

Compound (526) was obtained in a manner similar to Preparation 77.

¹H-NMR (300 MHz, CDCl₃, δ): 1.14-1.87 (10H, m), 1.39 (3H, t, J=7 Hz), 1.40 (3H, t, J=7 Hz), 1.90-2.16 (2H, m), 2.92-3.05 (2H, m), 3.16 (1H, dd, J=14, 6 Hz), 3.18 (1H, dd, J=14, 8 Hz), 3.55 (1H, dd, J=14, 10.5 Hz), 3.62 (1H, t, J=6 Hz), 3.70 (1H, ddd, J=10.5, 6, 6 Hz), 3.95 (1H, m), 3.98 (2H, q, J=7 Hz), 4.00 (2H, q, J=7 Hz), 4.19 (1H, m), 5.02 (1H, m), 5.29 (1H, m), 6.51 (1H, d, J=10.5 Hz), 6.67 (1H, d, J=6 Hz), 6.75 (2×1H, d, J=9 Hz), 6.82 (2×1H, d, J=8.5 Hz), 6.99 (2×1H, d, J=9 Hz), 7.15 (2×1H, d, J=8.5 Hz), 7.42 (1H, d, J=10 Hz);

MASS (ES+): m/e 623.98.

Preparation 527

Compound (527) was obtained in a manner similar to Preparation 78. The obtained compound was used in Examples 293 and 296.

¹H-NMR (300 MHz, CDCl₃, δ): 1.14-1.87 (8H, m), 1.39 (3H, t, J=7 Hz), 1.40 (3H, t, J=7 Hz), 1.90-2.16 (2H, m), 2.46 (2H, t, J=6.5 Hz), 2.91-3.06 (2H, m), 3.10-3.24 (2H, m), 3.53 (1H, dd, J=14, 10.5 Hz), 3.72 (1H, m), 3.94 (1H, m), 3.99 (2H, q, J=7 Hz), 4.00 (2H, q, J=7 Hz), 4.21 (1H, m), 5.02 (1H, m), 5.30 (1H, m), 6.50 (1H, d, J=10 Hz), 6.61 (1H, d, J=6 Hz), 6.76 (2×1H, d, J=8.5 Hz), 6.82 (2×1H, d, J=8.5 Hz), 6.99 (2×1H, d, J=8.5 Hz), 7.15 (2×1H, d, J=8.5 Hz), 7.34 (1H, d, J=10 Hz), 9.74 (1H, s);

MASS (ES+): m/e 621.45.

Preparation 528

Compound (528) was obtained in a manner similar to Preparation 119.

¹H-NMR (300 MHz, CDCl₃, δ): 1.24 (3H, t, J=7 Hz), 1.40 (3H, t, J=7 Hz), 3.02 (2H, m), 4.01 (2H, q, J=7 Hz), 4.16 (2H, q, J=7 Hz), 4.51 (1H, m), 4.96 (1H, brd, J=8 Hz), 6.81 (2×1H, d, J=8.4 Hz), 7.03 (2×1H, d, J=8.4 Hz);

MASS (ES+): m/e 338.51.

Preparation 529

Compound (529) was obtained in a manner similar to Preparation 17.

¹H-NMR (300 MHz, CDCl₃, δ): 1.40 (3H, t, J=7 Hz), 1.42 (3×3H, s), 3.08 (2H, m), 4.01 (2H, q, J=7 Hz), 4.54 (1H, m), 4.91 (8H, brd), 6.83 (2×1H, d, J=8.8 Hz), 7.09 (2×1H, d, J=8.8 Hz);

MASS (ES−): m/e 338.55.

Preparation 530

Compound (530) was obtained in a manner similar to Preparation 15.

¹H-NMR (300 MHz, CDCl₃, δ): 1.32-1.96 (4H, m), 1.38 (3H, t, J=7 Hz), 1.40 (3×3H, s), 2.56 (1H, m), 2.77 (1H, dd, J=13, 10 Hz), 2.82-3.09 (3H, m), 3.49 (1H, m), 3.98 (2H, q, J=7 Hz), 4.27-4.40 (2H, m), 4.83-5.03 (2H, m), 5.10 (1H, d, J=12 Hz), 5.18 (1H, d, J=12 Hz), 6.66 (1H, brd, J=8 Hz), 6.82 (2×1H, d, J=8.7 Hz), 7.08 (2×1H, d, J=8.7 Hz), 7.14-7.41 (10H, m);

MASS (ES+): m/e 644.50.

Preparation 531

Compound (531) was obtained in a manner similar to Preparation 16.

¹H-NMR (300 MHz, CDCl₃, δ): 1.22-2.28 (7H, m), 2.76 (1H, m), 2.85-3.34 (4H, m), 3.60 (1H, m), 3.74-4.04 (2H, m), 4.42 (1H, m), 4.68 (1H, m), 4.90-5.08 (2H, m), 5.17 (1H, d, J=12 Hz), 6.44-6.60 (2H, m), 6.73 (2×1H, d, J=8.5 Hz), 7.14-7.48 (10H, m), 7.86 (2H, br), 9.04 (1H, br);

MASS (ES+): m/e 544.50.

Preparation 532

Compound (532) was obtained in a manner similar to Preparation 16.

¹H-NMR (300 MHz, CDCl₃, δ): 1.29-1.95 (10H, m), 1.35 (3H, t, J=7 Hz), 1.43 (3×3H, S), 2.62 (1H, m), 2.72-3.06 (4H, m), 3.53 (1H, m), 3.95 (2H, q, J=7 Hz), 4.06 (1H, m), 4.27 (2H, t, J=6.5 Hz), 4.31 (1H, m), 4.66 (1H, m), 4.89 (1H, m), 5.10 (1H, d, J=12 Hz), 5.14 (1H, m), 5.16 (1H, d, J=12 Hz), 6.64-6.84 (2H, m), 6.80 (2×1H, d, J=8.8 Hz), 7.06 (2×1H, d, J=8.8 Hz), 7.12-7.47 (12H, m), 7.54 (1H, m), 8.03 (2×1H, dd, J=8, 1.5 Hz);

MASS (ES+): m/e 877.31.

Preparation 533

Compound (533) was obtained in a manner similar to Preparation 17.

¹H-NMR (300 MHz, CDCl₃, δ): 1.28-1.90 (9H, m), 1.36 (3H, t, J=7 Hz), 1.41 (3×3H, s), 2.09 (1H, m), 2.66 (1H, m), 2.84-3.05 (4H, m), 3.69 (1H, m), 3.96 (2H, q, J=7 Hz), 4.05 (1H, m), 4.21-4.36 (3H, m), 4.69 (1H, m), 4.80 (1H, m), 5.27 (1H, m), 6.78 (2×1H, d, J=8.7 Hz), 6.87 (1H, m), 7.04 (2×1H, brd, J=8.7 Hz), 7.13-7.33 (5H, m), 7.39-7.49 (3H, m), 7.55 (1H, m), 8.02 (2×1H, dd, J=8, 1.5 Hz);

MASS (ES+): m/e 787.42.

Preparation 534

Compound (534) was obtained in a manner similar to Preparation 18.

¹H-NMR (300 MHz, CDCl₃, δ): 1.16-1.96 (10H, m), 1.27 (3H, t, J=7 Hz), 2.70-3.14 (5H, m), 3.66 (1H, m), 3.84 (2H, q, J=7 Hz), 4.05-4.36 (4H, m), 4.59 (1H, m), 5.06 (1H, m), 6.73 (2×1H, d, J=8.5 Hz), 7.08-7.28 (8H, m), 7.40 (2×1H, dd, J=7.5, 7.5 Hz), 7.52 (1H, dd, J=7.5, 7.5 Hz), 7.95-8.32 (3H, m), 8.02 (2×1H, d, J=7.5 Hz);

MASS (ES+): m/e 687.52.

Preparation 535

Compound (535) was obtained in a manner similar to Preparation 76.

¹H-NMR (300 MHz, CDCl₃, δ): 1.34-1.52 (2H, m), 1.39 (3H, t, J=7 Hz), 1.56-1.95 (6H, m), 2.11-2.39 (2H, m), 2.77 (1H, dd, J=14, 7 Hz), 2.87 (1H, dd, J=13, 5 Hz), 3.02-3.24 (3H, m), 3.94 (1H, m), 3.98 (2H, q, J=7 Hz), 4.24-4.35 (2H, m), 4.61 (1H, dd, J=8, 2.5 Hz), 4.69 (1H, m), 5.06 (1H, m), 6.31 (1H, d, J=10 Hz), 6.46 (1H, d, J=10.5 Hz), 6.80 (2×1H, d, J=8.8 Hz), 7.11 (2×1H, d, J=8.8 Hz), 7.14-7.30 (6H, m), 7.44 (2×1H, dd, J=7.5, 7.5 Hz), 7.56 (1H, m), 8.03 (2×1H, dd, J=7.5, 1.5 Hz);

MASS (ES+): m/e 669.43.

Preparation 536

Compound (536) was obtained in a manner similar to Preparation 77.

¹H-NMR (300 MHz, CDCl₃, δ): 1.25-1.92 (8H, m), 1.40 (3H, t, J=7 Hz), 2.13-2.40 (2H, m), 2.77 (1H, dd, J=14, 7 Hz), 2.87 (1H, dd, J=13.5, 5 Hz), 3.02-3.24 (3H, m), 3.63 (2H, t, J=6.5 Hz), 3.94 (1H, m), 4.00 (2H, q, J=7 Hz), 4.28 (1H, m), 4.62 (1H, m), 4.69 (1H, m), 5.06 (1H, ddd, J=10, 10, 5 Hz), 6.40 (1H, d, J=10 Hz), 6.49 (1H, d, J=10 Hz), 6.81 (2×1H, d, J=8.7 Hz), 7.11 (2×1H, d, J=8.7 Hz), 7.15-7.32 (6H, m);

MASS (ES+): m/e 565.49.

Preparation 537

Compound (537) was obtained in a manner similar to Preparation 78. The obtained compound was used in Example 299.

¹H-NMR (300 MHz, CDCl₃, δ): 1.40 (3H, t, J=7 Hz), 1.48-1.90 (6H, m), 2.12-2.40 (2H, m), 2.45 (2H, m), 2.77 (1H, dd, J=14, 7 Hz), 2.87 (1H, dd, J=13.5, 5 Hz), 3.01-3.23 (3H, m), 3.94 (1H, m), 4.00 (2H, q, J=7 Hz), 4.28 (1H, dt, J=10, 7.5 Hz), 4.61 (1H, m), 4.68 (1H, m), 5.06 (1H, ddd, J=10, 10, 5 Hz), 6.32 (1H, d, J=10 Hz), 6.44 (1H, d, J=10 Hz), 6.80 (2×1H, d, J=8.7 Hz), 7.11 (2×1H, d, J=8.7 Hz), 7.14-7.31 (6H, m), 9.74 (1H, t, J=1.5 Hz);

MASS (ES+): m/e 563.49.

Preparation 538

Compound (538) was obtained in a manner similar to Preparation 15.

¹H-NMR (300 MHz, CDCl₃, δ): 1.30-1.85 (4H, m), 1.39 (3×3H, s), 2.54 (1H, m), 2.72 (1H, dd, J=12.8, 9.5 Hz), 2.85-3.02 (2H, m), 3.09 (1H, dd, J=14, 7 Hz), 3.48 (1H, m), 4.39 (1H, m), 4.90 (1H, m), 5.00 (1H, m), 5.10 (1H, d, J=12.5 Hz), 5.18 (1H, d, J=12.5 Hz), 6.63 (1H, brd, J=8.5 Hz), 7.12-7.40 (16H, m);

MASS (ES+): m/e 600.49.

Preparation 539

Compound (539) was obtained in a manner similar to Preparation 15.

¹H-NMR (300 MHz, CDCl₃, δ): 1.44-2.20 (4H, m), 2.66-2.90 (6H, m), 4.45 (1H, m), 4.72 (1H, m), 4.96 (1H, d, J=12 Hz), 5.02 (1H, m), 5.16 (1H, d, J=12 Hz), 7.01-7.50 (15H, m), 7.84-8.32 (3H, m);

MASS (ES+): m/e 500.50.

Preparation 540

Compound (540) was obtained in a manner similar to Preparation 16.

¹H-NMR (300 MHz, CDCl₃, δ): 1.20-1.95 (10H, m), 1.43 (3×3H, s), 2.60 (1H, m), 2.72-3.13 (4H, m), 3.52 (1H, m), 4.04 (1H, m), 4.20-4.34 (3H, m), 4.72 (1H, m), 4.88 (1H, m), 5.10 (1H, d, J=12.2 Hz), 5.13 (1H, m), 5.17 (1H, d, J=12.2 Hz), 6.72-6.83 (2H, m), 7.13-7.39 (15H, m), 7.42 (2×1H, dd, J=7.5, 7.5 Hz), 7.55 (1H, m), 8.03 (2×1H, dd, J=7.5, 1.5 Hz);

MASS (ES+): m/e 833.44.

Preparation 541

Compound (541) was obtained in a manner similar to Preparation 17.

¹H-NMR (300 MHz, CDCl₃, δ): 1.18-2.14 (10H, m), 2.66 (1H, m), 2.80-3.16 (4H, m), 3.69 (1H, m), 4.04 (1H, m), 4.20-4.34 (3H, m), 4.68-4.86 (2H, m), 5.28 (1H, brd, J=7.5 Hz), 6.92 (1H, brd, J=6 Hz), 7.08-7.31 (10H, m), 7.43 (2×1H, dd, J=7.5, 7.5 Hz), 7.49 (1H, brd, J=10 Hz), 7.55 (1H, m), 8.02 (2×1H, dd, J=7.5, 1.5 Hz);

MASS (ES+): m/e 743.43.

Preparation 542

Compound (542) was obtained in a manner similar to Preparation 18.

¹H-NMR (300 MHz, CDCl₃, δ): 1.10-1.97 (10H, m), 2.72-3.16 (5H, m), 3.66 (1H, m), 4.05-4.30 (4H, m), 4.60 (1H, m), 5.12 (1H, m), 7.10-7.36 (10H, m), 7.40 (2×1H, dd, J=7.5, 7.5 Hz), 7.52 (1H, dd, J=7.5, 7.5 Hz), 7.94-8.38 (4H, m), 8.03 (2×1H, d, J=7.5 Hz);

MASS (ES+): m/e 643.53.

Preparation 543

Compound (543) was obtained in a manner similar to Preparation 76.

¹H-NMR (300 MHz, CDCl₃, δ): 1.32-1.54 (2H, m), 1.57-1.95 (6H, m), 2.12-2.38 (2H, m), 2.84 (1H, dd, J=14, 7 Hz), 2.88 (1H, dd, J=13.5, 5 Hz), 3.08 (1H, m), 3.14-3.26 (2H, m), 3.94 (1H, m), 4.28 (2H, t, J=6.5 Hz), 4.29 (1H, m), 4.62 (1H, dd, J=8, 2.5 Hz), 4.75 (1H, m), 5.07 (1H, ddd, J=10, 10, 5 Hz), 6.35 (1H, d, J=10 Hz), 6.48 (1H, d, J=10 Hz), 7.13-7.31 (11H, m), 7.44 (2×1H, dd, J=7.5, 7.5 Hz), 7.56 (1H, m), 8.03 (2×1H, dd, J=7.5, 1.5 Hz);

MASS (ES+): m/e 625.28.

Preparation 544

Compound (544) was obtained in a manner similar to Preparation 77.

¹H-NMR (300 MHz, CDCl₃, δ): 1.20-1.91 (10H, m), 2.20 (1H, m), 2.31 (1H, m), 2.84 (1H, dd, J=14, 7 Hz), 2.87 (1H, dd, J=13.5, 5 Hz), 3.08 (1H, m), 3.18 (1H, dd, J=13.5, 10.5 Hz), 3.21 (1H, dd, J=14, 9 Hz), 3.62 (2H, t, J=6.5 Hz), 3.94 (1H, m), 4.28 (1H, dt, J=10, 7.5 Hz), 4.62 (1H, dd, J=8, 2.5 Hz), 4.74 (1H, m), 5.06 (1H, ddd, J=10.5, 10, 5 Hz), 6.48 (1H, d, J=10 Hz), 6.52 (1H, d, J=10 Hz), 7.13-7.34 (11H, m);

MASS (ES+): m/e 521.

Preparation 545

Compound (545) was obtained in a manner similar to Preparation 78. The obtained compound was used in Example 302.

¹H-NMR (300 MHz, CDCl₃, δ): 1.46-1.90 (6H, m), 2.20 (1H, m), 2.32 (1H, m), 2.44 (2H, m), 2.79-2.92 (2H, m), 3.08 (1H, m), 3.13-3.27 (2H, m), 3.94 (1H, m), 4.28 (1H, dt, J=10.2, 7.3 Hz), 4.62 (1H, dd, J=8, 2 Hz), 4.74 (1H, m), 5.06 (1H, ddd, J=10, 10, 5 Hz), 6.38 (1H, d, J=10 Hz), 6.47 (1H, d, J=10 Hz), 7.14-7.33 (11H, m), 9.73 (1H, t, J=1 Hz);

MASS (ES+): m/e 519.

Preparation 546

Compound (546) was obtained in a manner similar to Preparation 16.

¹H-NMR (300 MHz, CDCl₃, δ): 0.64-0.90 (6H, m), 1.12-2.00 (13H, m), 2.16 (1H, m), 2.46 (1H, m), 2.93-3.23 (5H, m), 3.85 (1H, m), 4.22 (2H, t, J=6.5 Hz), 4.30-4.64 (3H, m), 4.82 (⅔H, m), 5.02-5.23 (4+⅔H, m), 5.34 (⅔H, brd, J=7.5 Hz), 5.62 (⅓H, br), 6.34-6.60 (2H, m), 7.11-7.48 (17H, m), 7.55 (1H, m), 7.96-8.04 (2H, m);

MASS (ES+): m/e 847.48.

Preparation 547

Compound (547) was obtained in a manner similar to Preparation 17.

¹H-NMR (300 MHz, CDCl₃, δ): 0.60-2.23 (21H, m), 2.46-2.68 (2H, m), 3.16 (1H, m), 3.46 (1H, m), 4.16-4.36 (2H, m), 4.41-4.68 (3H, m), 4.81 (1H, m), 7.14-7.72 (8H, m), 7.99 (2×1H, dd, J=7.5, 1.5 Hz);

MASS (ES+): m/e 623.57.

Preparation 548

Compound (548) was obtained in a manner similar to Preparation 76.

¹H-NMR (300 MHz, CDCl₃, δ): 0.72 (3H, m), 0.78 (3H, d, J=6.3 Hz), 1.03-1.54 (6H, m), 1.58-1.98 (8H, m), 2.45 (1H, m), 2.70 (1H, m), 2.87 (1H, dd, J=13.7, 6.0 Hz), 3.24 (1H, dd, J=13.7, 9.8 Hz), 4.31 (2H, t, J=6.5 Hz), 4.44-4.70 (4H, m), 4.86 (1H, m), 5.99 (1H, br), 6.07 (1H, br), 6.27 (1H, d, J=10.7 Hz), 7.13-7.30 (5H, m), 7.43 (2×1H, dd, J=7.5, 7.5 Hz), 7.56 (1H, m), 8.02 (2×1H, dd, J=7.5, 1.5 Hz);

MASS (ES+): m/e 605.55.

Preparation 549

Compound (549) was obtained in a manner similar to Preparation 77.

¹H-NMR (300 MHz, CDCl₃, δ): 0.72 (3H, m), 0.78 (3H, d, J=6 Hz), 1.02-1.96 (14H, m), 2.46 (1H, m), 2.75 (1H, m), 2.88 (1H, dd, J=13.5, 6 Hz), 3.25 (1H, dd, J=13.5, 10 Hz), 3.63 (2H, t, J=6 Hz), 4.46-4.71 (4H, m), 4.89 (1H, m), 6.15 (1H, br), 6.29 (1H, br), 6.41 (1H, d, J=10.5 Hz), 7.14-7.35 (5H, m);

MASS (ES+): m/e 501.60.

Preparation 550

Compound (550) was obtained in a manner similar to Preparation 78. The obtained compound was used in Example 305.

¹H-NMR (300 MHz, CDCl₃, δ): 0.72 (3H, m), 0.79 (3H, d, J=6.7 Hz), 1.08 (1H, m), 1.18-1.96 (11H, m), 2.39-2.56 (3H, m), 2.76 (1H, m), 2.88 (1H, dd, J=13.5, 6 Hz), 3.25 (1H, dd, J=13.5, 10 Hz), 4.46-4.70 (4H, m), 4.87 (1H, m), 6.04-6.22 (2H, m), 6.31 (1H, d, J=10.5 Hz), 7.14-7.32 (5H, m), 9.76 (1H, s);

MASS (ES+): m/e 499.60.

Preparation 551

A solution of Compound (289) (300 mg) in a mixture of piperidine (1.2 ml) and N,N-dimethylformamide (4.8 ml) was stirred at ambient temperature for three hours. The mixture was concentrated in vacuo and the residue was purified by flash chromatography using ethyl acetate as a solvent to give the objective Compound (551) (275 mg) as a pale yellow oil.

¹H-NMR (300 MHz, CDCl₃, δ): 0.83 (3H, t, J=7.3 Hz), 1.28 (3H, s), 1.34-1.98 (8H, m), 2.07-2.23 (2H, m), 2.24-2.42 (2H, m), 2.83 (1H, dd, J=13.6, 5.9 Hz), 3.13 (1H, dd, J=13.6, 9.9 Hz), 3.19-3.34 (1H, m), 3.62 (2H, brs), 3.80-3.90 (1H, m), 4.18-4.29 (1H, m), 4.31 (2H, t, J=6.4 Hz), 4.67 (1H, brd, J=6.6 Hz), 5.11 (1H, dt, J=10.1, 5.9 Hz); 5.90 (1H, s), 6.60 (2H, d, J=8.4 Hz), 7.01 (2H, d, J=8.4 Hz), 7.18 (1H, d, J=10.3 Hz), 7.39-7.62 (4H, m), 7.99-8.06 (2H, m);

MASS (ES+): m/e 592.46 (M+1).

Preparation 552

To a stirred solution of the Compound (551) (540 mg) in pyridine (4 ml) was added methanesulfonyl chloride (110 mg) in an ice bath. The resulting mixture was stirred at the same temperature for two hours. The mixture was concentrated in vacuo and the residue was extracted with ethyl acetate, washed with water, 5% (w/v) potassium hydrogen sulfate, saturated aqueous sodium bicarbonate solution and brine. The organic layer was dried over magnesium sulfate, filtered and concentrated in vacuo. The residue was purified by flash chromatography using ethyl acetate as a solvent to give the objective Compound (552) (538 mg) as a pale yellow amorphous solid. The obtained compound was used in Example 90.

¹H-NMR (300 MHz, CDCl₃, δ): 0.81 (3H, t, J=7.5 Hz), 1.29 (3H, s), 1.35-2.00 (8H, m), 2.06-2.41 (4H, m), 2.96 (1H, dd, J=13.9, 6.6 Hz), 2.99 (3H, s), 3.21 (1H, dd, J=13.9, 9.5 Hz), 3.26-3.36 (1H, m), 3.79-3.92 (1H, m), 4.20-4.32 (1H, m), 4.32 (2H, t, J=6.4 Hz), 4.70 (1H, brd, J=7.3 Hz), 5.09-5.22 (1H, m), 5.97 (1H, s), 6.51 (1H, s), 7.10 (1H, d, J=10.0 Hz), 7.13 (2H, d, J=8.8 Hz), 7.23 (2H, d, J=8.8 Hz), 7.40-7.49 (2H, m), 7.52-7.66 (2H, m), 8.00-8.07 (2H, m);

MASS (ES+): m/e 670.53 (M+1).

Preparation 553

To a stirred solution of Compound (551) (260 mg) in pyridine (2 ml) was added acetic anhydride (1 ml) followed by a catalytic amount of 4-(dimethylamino)pyridine at ambient temperature, the resulting mixture was stirred at the same temperature for one hour. The volatiles were removed under reduced pressure and the residue was purified by flash chromatopraphy using ethyl acetate then 5% methanol/ethyl acetate (v/v) as a solvent mixture to give the objective Compound (553) (260 mg) as a pale yellow amorphous solid.

¹H-NMR (300 MHz, CDCl₃, δ): 0.82 (3H, t, J=7.3 Hz), 1.27 (3H, s), 1.36-1.98 (8H, m), 2.06-2.24 (2H, m), 2.16 (3H, s), 2.25-2.41 (2H, m), 2.91 (1H, dd, J=13.5, 5.7 Hz), 3.20 (1H, dd, J=13.5, 9.9 Hz), 3.21-3.34 (1H, m), 3.78-3.90 (1H, m), 4.18-4.30 (1H, m), 4.31 (2H, d, J=6.6 Hz), 4.66 (1H, brd, J=7.0 Hz), 5.14 (1H, dt, J=9.9, 5.9 Hz), 5.89 (1H, s), 7.12 (1H, d, J=9.9 Hz), 7.18 (2H, d, J=8.4 Hz), 7.40 (2H, d, J=8.4 Hz), 7.42-7.48 (2H, m), 7.50-7.60 (2H, m), 7.98-8.07 (2H, m);

MASS (ES+): m/e 634.73.

### EXAMPLE 1

To a stirred solution of dimethyl (3R)-tert-butyldimethylsilyloxy-2-oxobutylphosphonate (812 mg) in water and tetrahydrofuran (1:40) (7.5 ml) was added barium hydroxide octahydrate (482 mg) in one portion. The mixture was stirred at ambient temperature for 30 minutes. To the mixture was added a solution of Compound C1-3 (980 mg) in water and tetrahydrofuran (1:40) (1.5 ml once, 1 ml twice), and stirred for 1 hour. 10% Aqueous citric acid solution (50 ml) was added to the mixture to quench the reaction, stirred for 15 minutes under ice-cooling, and extracted with ethyl acetate (300 ml). The organic layer was washed with 10% citric acid (50 ml), water (50 ml) and brine (50 ml), dried over sodium sulfate and evaporated in vacuo. The residue was purified by flash column chromatography (eluting with ethyl acetate/hexane=2:3 to 1:1 v/v) to give Compound E1 as a white foam (852 mg).

¹H-NMR (300 MHz, CDCl₃, δ): 0.83 (3H, s), 1.09 (9H, s), 1.22 (3H, d, J=7.0 Hz), 1.28 (3H, s), 1.37-1.51 (2H, m), 1.54-1.89 (4H, m), 2.09-2.37 (6H, m), 2.89 (1H, dd, J=14.0, 6.2 Hz), 3.18 (1H, dd, J=14.0, 9.9 Hz), 3.19-3.29 (1H, m), 3.80-3.91 (1H, m), 4.15-4.28 (1H, m), 4.27 (1H, q, J=7.0 Hz), 4.63-4.70 (1H, m), 5.02 (2H, s), 5.06-5.19 (1H, m), 5.84 (1H, s), 6.61 (1H, d, J=15.4 Hz), 6.80-6.89 (1H, m), 6.88 (2H, d, J=8.5 Hz), 7.10-7.15 (1H, m), 7.14 (2H, d, J=8.5 Hz), 7.28-7.49 (11H, m), 7.51 (1H, d, J=10.7 Hz), 7.55-7.69 (4H, m);

MASS (ES+): m/e 885.56 (M+).

### EXAMPLE 2

Compound E2 was obtained in a manner similar to Example 1.

¹H-NMR (300 MHz, CDCl₃, δ): 0.83 (3H, t, J=6.7 Hz), 1.09 (9H, s), 1.23 (3H, d, J=6.5 Hz), 1.28 (3H, s), 1.35-1.53 (2H, m), 1.62-1.90 (3H, m), 2.09-2.38 (7H, m), 2.89 (1H, dd, J=13.5, 5.8 Hz), 3.18 (1H, dd, J=13.5, 9.9 Hz), 3.21-3.31 (1H, m), 3.81-3.92 (1H, m), 4.15-4.27 (1H, m), 4.27 (1H, q, J=6.5 Hz), 4.67 (1H, brd, J=5.6 Hz), 5.03 (2H, s), 5.08-5.19 (1H, m), 5.79 (1H, s), 6.61 (1H, d, J=15.8 Hz), 6.81-6.92 (1H, m), 6.88 (2H, d, J=8.8 Hz), 7.09-7.17 (1H, m), 7.14 (2H, d, J=8.8 Hz), 7.30-7.46 (11H, m), 7.50 (1H, d, J=10.7 Hz), 7.57-7.62 (2H, m), 7.63-7.69 (2H, m);

MASS (ES+): m/e 885.45 (M+).

### EXAMPLE 3

To a solution of the Compound E1 (86.9 ml) in methanol (3 ml), Pd—BaSO₄ (56.2 mg) was added and stirred for 1.25 hours under hydrogen atmosphere. The catalyst was filtered through a pad of Celite® and the solvent was evaporated under reduced pressure. The residue was purified by preparative thin layer chromatography to give Compound E3 as an oil (74.7 mg).

¹H-NMR (300 MHz, CDCl₃, δ): 0.83 (3H, t, J=7.3 Hz), 1.10 (9H, s), 1.26 (3H, d, J=6.6 Hz), 1.10-1.36 (6H, m), 1.27 (3H, s), 1.40-1.65 (3H, m), 1.67-1.85 (4H, m), 2.08-2.27 (2H, m), 2.27-2.40 (2H, m), 2.49 (2H, ddd, J=9.2, 7.0, 1.5 Hz), 2.88 (1H, dd, J=13.8, 5.9 Hz), 3.18 (1H, dd, J=13.8, 9.9 Hz), 3.18-3.30 (1H, m), 3.81-3.92 (1H, m), 4.14-4.24 (2H, m), 4.18 (1H, d, J=5.8 Hz), 5.02 (2H, s), 5.13 (1H, ddd, J=16.1, 9.9, 6.2 Hz), 5.84 (1H, s), 6.88 (2H, d, J=8.8 Hz), 7.07 (1H, d, J=10.3 Hz), 7.15 (2H, d, J=8.4 Hz), 7.25-7.45 (11H, m), 7.56 (1H, d, J=10.38 Hz), 7.55-7.68 (4H, m).

### EXAMPLE 4

To a solution of the Compound E1 in methanol-dioxane mixture (1:1) (20 ml) was added 10% palladium on carbon (300 mg) and the mixture was shaken under an atmosphere of hydrogen (4 atm) at ambient temperature for 20 hours. The mixture was filtered through a pad of Celite® and the filtrate was purified by flash chromatography (eluting with ethyl acetate/hexane=1:1 to 2:2 v/v) to give Compound E4 as a colorless amorphous compound (610 mg).

¹H-NMR (300 MHz, CDCl₃, δ): 0.83 (3H, t, J=7.3 Hz), 1.10 (9H, s), 1.14-1.56 (6H, m), 1.19 (3H, d, J=6.8 Hz), 1.28 (3H, s), 1.69-1.88 (4H, m), 2.07-2.24 (2H, m), 2.24-2.37 (2H, m), 2.45-2.56 (2H, m), 2.88 (1H, dd, J=13.5, 6.3 Hz), 3.16 (1H, dd, J=13.5, 9.8 Hz), 3.20-3.31 (1H, m), 3.77-3.89 (1H, m), 4.11-4.20 (1H, m), 4.18 (1H, q, J=6.8 Hz), 4.67 (1H, brd, J=6.8 Hz), 5.06-5.18 (1H, m), 5.10 (1H, s), 5.89 (1H, s), 6.73 (2H, d, J=8.4 Hz), 7.05-7.10 (1H, m), 7.09 (2H, d, J=8.4 Hz), 7.32-7.48 (6H, m), 7.53-7.70 (5H, m);

MASS (ES+): m/e 797.55 (M+).

### EXAMPLE 5

Compound E5 was obtained from the Compound E2 in a manner similar to Example 4.

¹H-NMR (300 MHz, CDCl₃, δ): 0.83 (3H, t, J=7.3 Hz), 1.10 (9H, s), 1.19 (3H, d, J=6.7 Hz), 1.21-1.61 (7H, m), 1.28 (3H, s), 1.69-1.88 (3H, m), 2.08-2.24 (2H, m), 2.25-2.38 (2H, m), 2.51 (2H, t, J=6.8 Hz), 2.89 (1H, dd, J=13.5, 6.2 Hz), 3.16 (1H, dd, J=13.5, 9.6 Hz), 3.21-3.31 (1H, m), 3.77-3.90 (1H, m), 4.08-4.24 (2H, m), 4.67 (1H, brd, J=5.9 Hz), 5.05-5.18 (1H, m), 5.20 (1H, s), 5.85 (1H, s), 7.04-7.10 (1H, m), 7.09 (2H, d, J=8.5 Hz), 7.32-7.48 (6H, m), 7.53-7.68 (5H, m);

MASS (ES+): m/e 797.57 (M).

### EXAMPLE 6

To a stirred solution of the Compound E3 (74.7 mg) in tetrahydrofuran (3 ml) was added tetrabutylammonium fluoride (1.0M in tetrahydrofuran, 0.1 ml) at ambient temperature and the mixture was stirred for 40 minutes at the same temperature. The reaction mixture was diluted with water (10 ml) and the organic layer was extracted with ethyl acetate (5 ml, twice). The combined organic layer was washed with brine (5 ml), dried over anhydrous sodium sulfate and filtered. The filtrate was concentrated under reduced pressure and the residue was purified by preparative thin layer chromatography (chloroform:methanol=10:1 v/v) to give Compound E6 (51.6 mg) as a colorless oil.

¹H-NMR (300 MHz, CDCl₃, δ): 0.83 (3H, t, J=7.3 Hz), 1.20-1.40 (4H, m), 1.28 (3H, s), 1.37 (3H, d, J=7.0 Hz), 1.56-1.70 (2H, m), 1.70-1.88 (2H, m), 2.08-2.24 (2H, m), 2.25-2.58 (4H, m), 2.89 (1H, dd, J=13.6, 5.9 Hz), 3.18 (1H, dd, J=13.6, 9.9 Hz), 3.19-3.30 (1H, m), 3.61 (1H, d, J=4.4 Hz), 3.80-3.90 (1H, m), 4.15-4.28 (2H, m), 4.68 (6.6H, d), 5.02 (2H, s), 5.15 (1H, ddd, J=16.1, 9.9, 6.2 Hz), 5.89 (1H, s), 6.88 (2H, d, J=8.8 Hz), 7.10-7.18 (3H, m), 7.25-7.45 (5H, m), 7.54 (1H, d, J=10.3 Hz);

MASS (ES+): m/e 648.35 (M+1).

### EXAMPLE 7

Compound E7 was obtained from the Compound E5 in a manner similar to Example 6.

¹H-NMR (300 MHz, CDCl₃, δ): 0.84 (3H, t, J=6.9 Hz), 1.22-1.69 (7H, m), 1.28 (3H, s), 1.38 (3H, d, J=7.1 Hz), 1.70-1.88 (3H, m), 2.07-2.24 (2H, m), 2.24-2.36 (2H, m), 2.88 (1H, dd, J=13.4, 5.5 Hz), 3.15 (1H, dd, J=13.4, 9.4 Hz), 3.20-3.32 (1H, m), 3.57 (1H, d, J=4.6 Hz), 3.77-3.89 (1H, m), 4.13-4.28 (2H, m), 4.68 (1H, brd, J=5.8 Hz), 5.05-5.18 (1H, m), 5.40 (1H, s), 5.89 (1H, s), 6.73 (2H, d, J=8.0 Hz), 7.09 (2H, d, J=8.0 Hz), 7.12 (1H, d, J=10.0 Hz), 7.55 (1H, d, J=10.2 Hz);

MASS (ES+): m/e 559.41 (M+1).

### EXAMPLE 8

Compound E8 was obtained from the Compound E4 in a manner similar to Example 6.

¹H-NMR (300 MHz, CDCl₃, δ): 0.84 (3H, t, J=7.1 Hz), 1.21-1.41 (4H, m), 1.29 (3H, s), 1.38 (3H, d, J=7.0 Hz), 1.53-1.69 (3H, m), 1.70-1.89 (3H, m), 2.06-2.23 (2H, m), 2.24-2.38 (2H, m), 2.39-2.55 (2H, m), 2.88 (1H, dd, J=13.5, 5.8 Hz), 3.15 (1H, dd, J=13.5, 9.6 Hz), 3.19-3.31 (1H, m), 3.57 (1H, d, J=4.7 Hz), 3.77-3.89 (1H, m), 4.07-4.29 (2H, m), 4.67 (1H, br d, J=6.5 Hz), 5.06-5.18 (1H, m), 5.29 (1H, s), 15.93 (1H, s), 6.73 (2H, d, J=8.5 Hz), 7.09 (2H, d, J=8.5 Hz), 7.12 (1H, d, J=10.0 Hz), 7.55 (1H, d, J=10.3 Hz);

MASS (ES+): m/e 559.31 (M+1).

### EXAMPLE 9

Compound E9 was obtained from the Compound (81) in a manner similar to Example 1.

¹H-NMR (300 MHz, CDCl₃, δ): 0.84 (3H, t, J=7.3 Hz), 1.09 (3×3H, s), 1.22 (3H, d, J=7 Hz), 1.28 (3H, s), 1.38-1.52 (2H, m), 1.56-1.90 (4H, m), 2.08-2.40 (6H, m), 2.89 (1H, dd, J=14, 6 Hz), 3.18 (1H, dd, J=14, 10 Hz), 3.26 (1H, m), 3.77 (3H, s), 3.86 (1H, m), 4.21° (1H, m), 4.26 (1H, q, J=7 Hz), 4.66 (1H, m), 5.14 (1H, ddd, J=10, 10, 6 Hz), 5.84 (1H, s), 6.62 (1H, brd, J=16 Hz), 6.81 (2×1H, d, J=8.5 Hz), 6.84 (1H, dt, J=16, 7 Hz), 7.14 (2×1H, d, J=8.5 Hz), 7.29-7.45 (6H, m), 7.51 (1H, d, J=10 Hz), 7.55-7.68 (4H, m);

MASS (ES−): m/e 807.

### EXAMPLE 10

Compound E10 was obtained from the Compound (80) in a manner similar to Example 2.

¹H-NMR (300 MHz, CDCl₃, δ): 0.83 (3H, t, J=7.8 Hz), 1.21 (9H, s), 1.26 (3H, d, J=6.9 Hz), 1.63 (3H, s), 1.70-1.58 (4H, m), 1.71-1.79 (3H, m), 2.09-2.39 (6H, m), 2.89 (1H, dd, J=13.8, 5.7 Hz), 3.18 (1H, dd, J=13.8, 9.6 Hz), 3.22-3.31 (1H, m), 3.77 (3H, s), 3.79-3.92 (1H, m), 4.18-4.27 (1H, m), 4.27 (1H, q, J=6.9 Hz), 5.13 (1H, ddd, J=9.9, 9.9, 5.7 Hz), 5.84 (1H, s), 6.61 (1H, d, J=15.3 Hz), 6.81 (2H, d, J=8.7 Hz), 6.86 (1H, dt, J=15.3, 6.9 Hz), 7.15 (2H, d, J=8.7 Hz), 7.31-7.48 (5H, m), 7.51 (1H, d, J=10.5 Hz), 7.57-7.69 (5H, m);

MASS (ES+): m/e 809.48 (M).

### EXAMPLE 11

Compound E11 was obtained from the Compound E9 in a manner similar to Example 3.

¹H-NMR (300 MHz, CDCl₃, δ): 0.84 (3H, t, J=7.3 Hz), 1.10 (3×3H, s), 1.18 (3H, d, J=7 Hz), 1.20-1.30 (4H, m), 1.28 (3H, s), 1.40-1.51 (2H, m), 1.60 (1H, m), 1.68-1.88 (3H, m), 2.09-2.24 (2H, m), 2.25-2.38 (2H, m), 2.51 (2H, m), 2.89 (1H, dd, J=13.5, 6 Hz), 3.18 (1H, dd, J=13.5, 10 Hz), 3.26 (1H, m), 3.85 (1H, m), 4.18 (1H, m), 4.18 (1H, q, J=7 Hz), 4.67 (1H, m), 5.13 (1H, ddd, J=10, 10, 6 Hz), 5.85 (1H, s), 6.81 (2×1H, d, J=8.5 Hz), 7.08 (1H, d, J=10 Hz), 7.14 (2×1H, d, J=8.5 Hz), 7.33-7.48 (6H, m), 7.56 (1H, d, J=10 Hz), 7.59-7.68 (4H, m);

MASS (ES+): m/e 811.

### EXAMPLE 12

Compound E12 was obtained from the Compound E10 in a manner similar to Example 3.

¹H-NMR (300 MHz, CDCl₃, δ): 0.84 (3H, t, J=7.5 Hz), 1.10 (9H, s), 1.16-1.32 (11H, m), 1.18 (3H, d, J=6.6 Hz), 1.38-1.51 (1H, m), 1.61 (3H, s), 1.68-1.88 (2H, m), 2.08-2.24 (2H, m), 2.25-2.39 (2H, m), 2.50 (2H, t), 2.89 (1H, dd, J=13.5, 6.0 Hz), 3.18 (1H, dd, J=13.5, 9.9 Hz), 3.23-3.30 (1H, m), 3.77 (3H, s), 3.81-3.90 (1H, m), 4.13-4.23 (1H, m), 4.18 (1H, q, J=6.6 Hz), 4.64-4.69 (1H, m), 5.13 (1H, ddd, J=9.9, 9.9, 6.3 Hz), 5.84 (1H, s), 6.81 (2H, d, J=8.7 Hz), 7.08 (1H, d, J=9.9 Hz), 7.15 (2H, d, J=8.7 Hz), 7.33-7.48 (6H, m), 7.55 (1H, d, J=10.2 Hz);

MASS (ES+): m/e 811.49.

### EXAMPLE 13

Compound E13 was obtained from the Compound E11 in a manner similar to Example 6.

¹H-NMR (300 MHz, CDCl₃, δ): 0.84 (3H, t, J=7.3 Hz), 1.20-1.40 (4H, m), 1.29 (3H, s), 1.38 (3H, d, J=7 Hz), 1.54-1.69 (3H, m), 1.70-1.90 (3H, m), 2.08-2.23 (2H, m), 2.26-2.56 (4H, m), 2.89 (1H, dd, J=14, 6 Hz), 3.18 (1H, dd, J=14, 10 Hz), 3.26 (1H, m), 3.56 (1H, d, J=5 Hz), 3.86 (1H, m), 4.14-4.30 (2H, m), 4.67 (1H, m), 5.13 (1H, ddd, J=10, 10, 6 Hz), 5.87 (1H, s), 6.81 (2×1H, d, J=9 Hz), 7.12 (1H, d, J=11 Hz), 7.14 (2×1H, d, J=9 Hz), 7.53 (1H, d, J=10 Hz);

MASS (ES−): m/e 571;

[α]_{D}²⁵=−116.5° (c=0.31, CHCl₃).

### EXAMPLE 14

Compound E14 was obtained from the Compound E12 in a manner similar to Example 6.

¹H-NMR (300 MHz, CDCl₃, δ): 0.83 (3H, t, J=6.9 Hz), 1.23-1.40 (2H, m), 1.38 (3H, d, J=7.2 Hz), 1.55-1.90 (6H, m), 1.64 (3H, s), 2.05-2.58 (6H, m), 2.88 (1H, dd, J=13.5, 6.0 Hz), 3.18 (1H, dd, J=13.5, 9.9 Hz), 3.21-3.30 (1H, m), 3.55 (1H, d, J=4.8 Hz), 3.78 (3H, s), 3.80-3.90 (1H, m), 4.16-4.28 (1H, m), 4.19 (1H, q, 7.2 Hz), 4.64-4.70 (1H, m), 5.13 (1H, ddd, J=9.9, 9.9, 6.0 Hz), 5.89 (1H, s), 6.81 (2H, d, J=8.4 Hz), 7.12 (1H, d, J=9.3 Hz), 7.14 (2H, d, J=8.4 Hz), 7.53 (1H, d, J=10.2 Hz);

MASS (ES+): m/e 573.49 (M+1).

### EXAMPLE 15

Compound E15 was obtained from the Compound (84) in a manner similar to Example 1.

¹H-NMR (300 MHz, CDCl₃, δ): 0.83 (3H, t, J=7.0 Hz), 1.10 (9H s), 1.23 (3H, d, J=6.9 Hz), 1.29 (3H, s), 1.36-1.55 (2H, m), 1.63-1.90 (4H, m), 2.07-2.39 (6H, m), 2.95 (1H, dd, J=13.9, 7.4 Hz), 3.21 (1H, dd, J=13.9, 8.7 Hz), 3.22-3.34 (1H, m), 3.80-3.91 (1H, m), 4.18-4.29 (1H, m), 4.28 (1H, q, J=6.9 Hz), 4.68 (1H, brd, J=7.1 Hz), 5.08-5.20 (1H, m), 5.83 (1H, s), 6.62 (1H, d, J=15.7 Hz), 6.82-6.98 (1H, m), 6.97 (2H, t, J=8.7 Hz), 7.09 (1H, d, J=10.6 Hz), 7.20 (2H, dd, J=8.7, 5.4 Hz), 7.29-7.48 (6H, m), 7.55 (1H, d, J=10.6 Hz), 7.56-7.69 (4H, m);

MASS (ES+): m/e 797.59 (M+1).

### EXAMPLE 16

Compound E16 was obtained from the Compound E15 in a manner similar to Example 3 except that 10% palladium on carbon was used instead of Pd—BaSO₄.

¹H-NMR (300 MHz, CDCl₃, δ): 0.83 (3H, t, J=7.3 Hz), 1.10 (9H, s), 1.16-1.32 (3H, m), 1.18 (3H, d, J=6.7 Hz), 1.28 (3H, s), 1.38-1.62 (4H, m), 1.72-1.88 (3H, m), 2.09-2.38 (4H, m), 2.46-2.55 (2H, m), 2.93 (1H, dd, J=13.2, 7.1 Hz), 3.20 (1H, dd, J=13.2, 8.7 Hz), 3.22-3.32 (1H, m), 3.79-3.89 (1H, m), 4.12-4.24 (1H, m), 4.19 (1H, q, J=6.7 Hz), 4.67 (1H, brd, J=5.4 Hz), 5.08-5.19 (1H, m), 5.83 (1H, s), 6.96 (2H, t, J=8.6 Hz), 7.04 (1H, d, J=10.2 Hz), 7.19 (2H, dd, J=8.6, 5.5 Hz), 7.32-7.48 (6H, m), 7.54-7.67 (5H, m);

MASS (ES+): m/e 799.52 (M).

### EXAMPLE 17

Compound E17 was obtained from the Compound E16 in a manner similar to Example 6.

¹H-NMR (300 MHz, CDCl₃, δ): 0.83 (3H, t, J=7.3 Hz), 1.24-1.39 (6H, m), 1.28 (3H, s), 1.38 (3H, d, J=7.2 Hz), 1.54-1.69 (1H, m), 1.71-1.89 (3H, m), 2.08-2.58 (6H, m), 2.93 (1H, dd, J=13.9, 6.3 Hz), 3.20 (1H, dd, J=13.9, 9.6 Hz), 3.21-3.32 (1H, m), 3.55 (1H, d, J=4.7 Hz), 3.78-3.91 (1H, m), 4.14-4.29 (2H, m), 4.68 (1H, brd, J=5.8 Hz), 5.08-5.19 (1H, m), 5.87 (1H, s), 6.96 (2H, t, J=8.8 Hz), 7.07 (1H, d, J=10.4 Hz), 7.19 (2H, dd, J=8.8, 5.5 Hz), 7.56 (1H, d, J=10.7 Hz);

MASS (ES+): m/e 561.46 (M+1).

### EXAMPLE 18

Compound E18 was obtained from the Compound (87) in a manner similar to Example 1.

¹H-NMR (300 MHz, CDCl₃, δ): 1.09 (9H, s), 1.22 (1H, d, J=7.2 Hz), 1.37-1.88 (15H, m), 2.12-2.38 (3H, m), 2.43-2.58 (2H, m), 2.95 (1H, dd, J=13.5, 6.0 Hz), 3.25 (1H, dd, J=13.5, 10.2 Hz), 3.28-3.13 (1H, m), 3.85-3.95 (1H, m), 4.22 (1H, dt, J=10.2, 7.8 Hz), 4.27 (1H, q, J=7.2 Hz), 4.64-4.69 (1H, m), 5.15 (1H, ddd, J=9.9, 9.9, 5.7 Hz), 6.16 (1H, s), 6.61 (1H, d, J=15.6 Hz), 6.87 (1H, dt, J=15.6, 6.9 Hz), 7.16-7.33 (5H, m), 7.33-7.48 (8H, m), 7.57-7.74 (4H, m);

MASS (ES+): m/e 791.60 (M).

### EXAMPLE 19

Compound E19 was obtained from the Compound E18 in a manner similar to Example 3 except that 10% palladium on carbon was used instead of Pd—BaSO₄.

¹H-NMR (300 MHz, CDCl₃, δ): 1.10 (9H, s), 1.01-1.84 (17H, m), 1.18 (3H, d, J=6.9 Hz), 2.11-2.36 (2H, m), 2.41-2.58 (3H, m), 2.95 (1H, dd, J=10.5, 6.0 Hz), 3.15-3.26 (1H, m), 3.26 (1H, dd, J=10.5, 13.5 Hz), 3.84-3.94 (1H, m), 4.12 (1H, dt, J=6.9, 7.5 Hz), 4.18 (1H, q, J=6.9 Hz), 4.63-4.69 (1H, m), 5.14 (1H, ddd, J=9.6, 9.6, 6.0 Hz), 6.14 (1H, s); 7.13 (1H, d, J=10.2 Hz), 7.17-7.31 (4H, m), 7.32-7.49 (8H, m), 7.57-7.66 (4H, m);

MASS (ES+): m/e 793.57 (M).

### EXAMPLE 20

Compound E20 was obtained from the Compound E19 in a manner similar to Example 6.

¹H-NMR (300 MHz, CDCl₃, δ): 1.19-1.87 (17H, m), 1.38 (3H, d, J=7.2 Hz), 2.11-2.23 (1H, m), 2.24-2.39 (2H, m), 2.40-2.58 (2H, m), 2.95 (1H, dd, J=13.5, 6.0 Hz), 3.15-3.25 (1H, m), 3.25 (1H, dd, J=13.5, 10.2 Hz), 3.56 (1H, d, J=4.8 Hz), 3.86-3.95 (1H, m), 4.12 (1H, q, J=7.2 Hz), 4.28-4.12 (1H, m), 4.63-4.69 (1H, m), 5.15 (1H, ddd, J=10.2, 10.2, 6.0 Hz), 6.18 (1H, s), 7.14-7.34 (6H, m), 7.43 (1H, d, J=10.2 Hz);

MASS (ES+): m/e 555.41 (M+1).

### EXAMPLE 21

Compound 21 was obtained from the Compound (90) in a manner similar to Example 1.

¹H-NMR (300 MHz, CDCl₃, δ): 0.812 (3H, t, J=7.2 Hz), 1.10 (6H, s), 1.11 (3H, s), 1.27 (3H, s), 1.37-1.91 (8H, m), 2.08-2.39 (6H, m), 3.06 (1H, dd, J=14.7, 6.9 Hz), 3.25-3.36 (1H, m), 3.27 (1H, dd, J=14.7, 8.7 Hz), 3.80-3.89 (1H, m), 4.18-4.31 (1H, m), 4.26 (2H, t, J=6.6 Hz), 4.66-4.71 (1H, m), 5.13-5.23 (1H, m), 5.89 (1H, s), 6.62 (1H, d, J=15.9 Hz), 6.87 (1H, dt, J=15.9, 6.9 Hz), 7.01 (1H, d, J=10.8 Hz), 7.30-7.49 (7H, m), 7.56-7.68 (8H, m);

MASS (ES+): m/e 804.62 (M+1).

### EXAMPLE 22

Compound E22 was obtained from the Compound E21 in a manner similar to Example 3 except that 10% palladium on carbon was used instead of 5% Pd—BaSO₄.

¹H-NMR (300 MHz, CDCl₃, δ): 0.807 (3H, t, J=6.9 Hz), 1.10 (9H, s), 1.28 (3H, s), 1.38-1.90 (11H, m), 2.06-2.39 (6H, m), 2.51 (2H, dt, J=7.2, 2.7 Hz), 3.06 (1H, dd, J=13.5, 7.5 Hz), 3.26-3.36 (1H, m), 3.27 (1H, dd, J=13.5, 9.0 Hz), 3.79-3.88 (1H, m), 4.19 (1H, dq, J=6.6, 2.7 Hz), 4.25 (1H, dt, J=13.8, 6.9 Hz), 4.66-4.71 (1H, m), 5.18 (1H, dt, J=9.6, 8.1 Hz), 5.87 (1H, s), 6.95 (1H, d, J=10.2 Hz), 7.32-7.49 (7H, m), 7.58-7.69 (7H, m), 7.58 (1H, d, J=9.0 Hz);

MASS (ES+): m/e 806.38 (M+1).

### EXAMPLE 23

Compound E23 was obtained from the Compound E22 in a manner similar to Example 6.

¹H-NMR (300 MHz, CDCl₃, δ): 0.811 (3H, t, J=7.5 Hz), 1.24-1.68 (11H, m), 1.38 (3H, d, J=7.2 Hz), 1.75-1.89 (3H, m), 2.06-2.57 (6H, m), 3.06 (1H, dd, J=14.1, 7.5 Hz), 3.26-3.36 (1H, m), 3.26 (1H, dd, J=14.1, 8.7 Hz), 3.79-3.88 (1H, m), 4.15-4.28 (2H, m), 4.65-4.71 (1H, m), 5.18 (1H, dt, J=8.4, 7.2 Hz), 5.90 (1H, s), 6.99 (1H, d, J=10.5 Hz), 7.33-7.39 (2H, m), 7.56-7.61 (2H, m), 7.63 (1H, d, J=10.2 Hz);

MASS (ES+): m/e 568.50 (M+1).

### EXAMPLE 24

Compound E24 was obtained from the Compound (93) in a manner similar to Example 1.

¹H-NMR (300 MHz, CDCl₃, δ): 0.84 (3H, t, J=7.2 Hz), 1.09 (5H, s), 1.10 (4H, s), 1.22 (3H, d, J=6.9 Hz), 1.28 (3H, s), 1.37-1.90 (8H, m), 1.39 (3H, t, J=6.9 Hz), 2.10-2.38 (4H, m), 2.88 (1H, dd, J=13.5, 5.7 Hz), 3.19 (1H, dd, J=13.5, 9.6 Hz), 3.12-3.30 (1H, m), 3.81-3.90 (1H, m), 3.99 (2H, q, J=6.9 Hz), 4.16-4.31 (2H, m), 4.64-4.69 (1H, m), 5.13 (1H, dt, J=9.6, 5.7 Hz), 5.85 (1H, s), 6.61 (1H, d, J=15.9 Hz), 6.79 (2H, d, J=8.4 Hz), 6.86 (1H, dt, J=15.9 Hz), 7.12-7.17 (1H, m), 7.13 (2H, d, J=8.4 Hz), 7.31-7.47 (5H, m), 7.50 (1H, d, J=10.2 Hz), 7.56-7.68 (5H,

MASS (ES+): m/e 823.64 (M+1).

### EXAMPLE 25

Compound E25 was obtained from the Compound E24 in a manner similar to Example 16.

¹H-NMR (300 MHz, CDCl₃, δ): 0.85 (3H, t, J=7.2 Hz), 1.11 (9H, s), 1.20 (3H, d, J=6.9 Hz), 1.20-1.65 (7H, m), 1.29 (3H, s), 1.40 (3H, t, J=6.9 Hz), 1.71-1.86 (3H, m), 2.09-2.24 (2H, m), 2.26-2.38 (2H, m), 2.52 (1H, dt, J=7.5, 2.1 Hz), 2.89 (1H, dd, J=13.5, 5.7 Hz), 3.13-3.31 (1H, m), 3.23 (1H, dd, J=13.5, 9.6 Hz), 3.81-3.90 (1H, m), 4.00 (1H, q, J=6.9 Hz), 4.19 (1H, dq, J=6.9, 2.1 Hz), 4.64-4.70 (1H, m), 5.14 (1H, dt, J=9.6, 5.7 Hz), 5.83 (1H, s), 6.80 (2H, d, J=8.7 Hz), 7.10 (1H, d, J=11.1 Hz), 7.14 (2H, d, J=8.7 Hz), 7.34-7.48 (5H, m), 7.55 (1H, d, J=10.5 Hz), 7.60-7.67 (5H, m);

MASS (ES+): m/e 825.65 (M+1).

### EXAMPLE 26

Compound E26 was obtained from the Compound E25 in a manner similar to Example 6.

¹H-NMR (300 MHz, CDCl₃, δ): 0.84 (3H, t, J=6.9 Hz), 1.20-1.42 (7H, m), 1.28 (3H, s), 1.39 (3H, t, J=7.2 Hz), 1.52-1.69 (3H, m), 1.71-1.87 (3H, m), 2.08-2.24 (2H, m), 2.26-2.39 (2H, m), 2.46 (2H, dt, J=11.7, 7.2 Hz), 2.88 (1H, dd, J=13.2, 5.7 Hz), 3.17 (1H, dd, J=13.2, 11.2 Hz), 3.22-3.30 (1H, m), 3.55 (1H, d, J=4.5 Hz), 3.81-3.90 (1H, m), 3.99 (2H, q, J=7.2 Hz), 4.14-4.28 (2H, m), 4.64-4.69 (1H, m), 5.13 (1H, dt, J=11.2, 5.7 Hz), 5.84 (1H, s), 7.08-7.16 (1H, m), 7.13 (2H, d, J=8.4 Hz), 7.52 (1H, d, J=10.5 Hz);

MASS (ES+): m/e 587.56 (M+1).

### EXAMPLE 27

Compound E27 was obtained from the Compound (96) in a manner similar to Example 1.

¹H-NM (300 MHz, CDCl₃, δ): 0.79 (3H, t, J=7 Hz), 1.09 (3×3H, s), 1.22 (3H, d, J=7 Hz), 1.26 (3H, s), 1.45 (2H, m), 1.65 (1H, m), 1.74-1.93 (3H, m), 2.10-2.40 (6H, m), 3.11 (1H, dd, J=15, 8 Hz), 3.15 (1H, dd, J=15, 8 Hz), 3.40 (1H, m), 3.88 (1H, m), 4.21 (1H, m), 4.27 (1H, q, J=7 Hz), 4.69 (1H, m), 5.24 (1H, ddd, J=9, 8, 8 Hz), 5.80 (1H, s), 6.62 (1H, d, J=16 Hz), 6.87 (1H, dt, J=16, 7 Hz), 6.96-7.13 (3H, m), 7.15-7.27 (2H, m), 7.30-7.48 (6H, m), 7.52 (3H, d, J=9-Hz), 7.55-7.70 (4H, m);

MASS (ES−): m/e 795.

### EXAMPLE 28

Compound E28 was obtained from the Compound (96) in a manner similar to Example 2.

¹H-NMR (300 MHz, CDCl₃, δ): 0.78 (3H, t, J=7 Hz), 1.09 (3×3H, s), 1.22 (3H, d, J=7 Hz), 1.26 (3H, s), 1.45 (2H, m), 1.65 (1H, m), 1.72-1.92 (3H, m), 2.10-2.40 (6H, m), 3.11 (1H, dd, J=15, 8 Hz), 3.15 (1H, dd, J=15, 8 Hz), 3.40 (1H, m), 3.88 (1H, m), 4.21 (1H, m), 4.27 (1H, q, J=7 Hz), 4.70 (1H, dd, J=8, 2 Hz), 5.23 (1H, ddd, J=9, 8, 8 Hz), 5.78 (1H, s), 6.61 (1H, d, J=16 Hz), 6.86 (1H, dt, J=16, 7 Hz), 6.96-7.12 (3H, m), 7.15-7.28 (2H, m), 7.30-7.48 (6H, m), 7.52 (1H, d, J=9 Hz), 7.55-7.69 (4H, m);

MASS (ES−): m/e 795.

### EXAMPLE 29

Compound E29 was obtained from the Compound (96) in a manner similar to Example 1 except that dimethyl (3R)-tert-butyldimethylsilyloxy-2-oxopentylphosphonate was used instead of dimethyl (3R)-tert-butyldimethylsilyloxy-2-oxobutylphosphonate.

¹H-NMR (300 MHz, CDCl₃, δ): 0.79 (3H, t, J=7. Hz), 0.80 (3H, t, J=7 Hz), 1.10 (3×3H, s), 1.26 (3H, s), 1.42 (2H, m), 1.55-1.70 (3H, m), 1.72-1.91 (3H, m), 2.10-2.41 (6H, m), 3.11 (1H, dd, J=14, 8 Hz), 3.15 (1H, dd, J=14, 8 Hz), 3.41 (1H, m), 3.89 (1H, m), 4.14 (1H, q, J=7 Hz), 4.21 (1H, m), 4.69 (1H, m), 5.24 (1H, ddd, J=10, 8, 8 Hz), 5.78 (1H, s), 6.55 (1H, d, J=16 Hz), 6.80 (1H, dt, J=16, 7 Hz), 6.97-7.12 (3H, m), 7.15-7.27 (2H, m), 7.29-7.47 (6H, m), 7.52 (1H, d, J=10 Hz), 7.55-7.67 (4H, m);

MASS (ES−): m/e 809.

### EXAMPLE 30

Compound E30 was obtained from the Compound E27 in a manner similar to Example 3 except that 10% palladium on carbon was used instead of Pd—BaSO₄.

¹H-NMR (300 MHz, CDCl₃, δ): 0.79 (3H, t, J=7 Hz), 1.10 (3×3H, s), 1.15-1.34 (4H, m), 1.18 (3H, d, J=7 Hz), 1.45 (2H, m), 1.60 (1H, m), 1.72-1.92 (3H, m), 2.08-2.40 (4H, m), 2.50 (2H, m), 3.10 (1H, dd, J=15, 8 Hz), 3.15 (1H, dd, J=15, 7.5 Hz), 3.41 (1H, m), 3.87 (1H, m), 4.18 (1H, m), 4.18 (1H, q, J=7 Hz), 4.69 (1H, m), 5.23 (1H, ddd, J=10, 8, 7.5 Hz), 5.80 (1H, s), 6.96-7.08 (3H, m), 7.15-7.27 (2H, m), 7.32-7.49 (6H, m), 7.55 (1H, d, J=10 Hz), 7.55-7.70 (5H, m);

MASS (ES−): m/e 797.

### EXAMPLE 31

Compound E31 was obtained from the Compound E30 in a manner similar to Example 3 except that 10% palladium on carbon was used instead of Pd—BaSO₄.

¹H-NMR (300 MHz, CDCl₃, δ): 0.79 (3H, t, J=7 Hz), 1.10 (3×3H, s), 1.15-1.32 (4H, m), 1.18 (3H, d, J=7 Hz), 1.45 (2H, m), 1.60 (1H, m), 1.71-1.92 (3H, m), 2.09-2.40 (4H, m), 2.51 (2H, t, J=7 Hz), 3.10 (1H, dd, J=15, 8 Hz), 3.15 (1H, dd, J=15, 8 Hz), 3.40 (1H, m), 3.87 (1H, m), 4.18 (1H, q, J=7 Hz), 4.18 (1H, m), 4.69 (1H, m), 5.23 (1H, ddd, J=10, 8, 7.5 Hz), 5.79 (1H, s), 6.95-7.09 (3H, m), 7.14-7.28 (2H, m), 7.32-7.49 (6H, m), 7.55 (1H, d, J=10 Hz), 7.55-7.68 (6H, m);

MASS (ES−): m/e 797.

### EXAMPLE 32

Compound E32 was obtained from the Compound E29 in a manner similar to Example 3 except that 10% palladium on carbon was used instead of Pd—BaSO₄.

¹H-NMR (300 MHz, CDCl₃, δ): 0.79 (3H, t, J=7 Hz), 0.81 (3H, t, J=7 Hz), 1.11 (3×3H, s), 1.13-1.28 (4H, m), 1.26 (3H, s), 1.37 (2H, m), 1.49-1.67 (3H, m), 1.71-1.92 (3H, m), 2.08-2.49 (6H, m), 3.10 (1H, dd, J=15, 8 Hz), 3.15 (1H, dd, J=15, 7.5 Hz), 3.40 (1H, m), 3.87 (1H, m), 4.10 (1H, t, J=6 Hz), 4.17 (1H, m), 4.69 (1H, m), 5.23 (1H, ddd, J=9, 8, 7.5 Hz), 5.79 (1H, s), 6.96-7.08 (3H, m), 7.14-7.28 (2H, m), 7.32-7.47 (6H, m), 7.55 (1H, d, J=9 Hz), 7.55-7.66 (5H, m);

MASS (ES−): m/e 811.

### EXAMPLE 33

Compound E33 was obtained from the Compound E30 in a manner similar to Example 6.

¹H-NMR (300 MHz, CDCl₃, δ): 0.80 (3H, t, J=7.5 Hz), 1.24-1.42 (4H, m), 1.26 (3H, s), 1.38 (3H, d, J=7 Hz), 1.54-1.70 (3H, m), 1.74-1.92 (3H, m), 2.08-2.58 (6H, m), 3.11 (1H, dd, J=15, 8 Hz), 3.15 (1H, dd, J=15, 7 Hz), 3.41 (1H, m), 3.58 (1H, d, J=5 Hz), 3.87 (1H, m), 4.13-4.30 (2H, m), 4.70 (1H, m), 5.24 (1H, ddd, J=10, 8, 7 Hz), 5.84 (1H, s), 6.97-7.12 (3H, m), 7.15-7.30 (2H, m), 7.54 (1H, d, J=10 Hz);

MASS (ES−): m/e 559;

MASS (ES+): m/e 561.

### EXAMPLE 34

Compound E34 was obtained from the Compound E31 in a manner similar to Example 6.

¹H-NMR (300 MHz, CDCl₃, δ): 0.80 (3H, t, J=7.5 Hz), 1.20-1.42 (4H, m), 1.26 (3H, s), 1.38 (3H, d, J=7 Hz), 1.53-1.73 (3H, m), 1.74-1.93 (3H, m), 2.09-2.59 (6H, m), 3.10 (1H, dd, J=15, 8 Hz), 3.15 (1H, dd, J=15, 7 Hz), 3.40 (1H, m), 3.56 (1H, d, J=5 Hz), 3.87 (1H, m), 4.14-4.29 (2H, m), 4.70 (1H, m), 5.24 (1H, ddd, J=10, 8, 7 Hz), 5.83 (1H, s), 6.96-7.13 (3H, m), 7.15-7.29 (2H, m), 7.54 (1H, d, J=10 Hz);

MASS (ES−): m/e 559.

### EXAMPLE 35

Compound E35 was obtained from the Compound E32 in a manner similar to Example 6.

¹H-NMR (300 MHz, CDCl₃, δ): 0.79 (3H, t, J=7.5 Hz), 0.94 (3H, t, J=7.5 Hz), 1.17-1.40 (4H, m), 1.26 (3H, s), 1.50-1.78 (4H, m), 1.79-1.97 (4H, m), 2.08-2.40 (6H, m), 2.45 (2H, m), 3.10 (1H, dd, J=15, 7.5 Hz), 3.14 (1H, dd, J=15, 7.5 Hz), 3.40 (1H, m), 3.51 (1H, d, J=5 Hz), 3.87 (1H, m), 4.08-4.26 (2H, m), 4.70 (1H, m), 5.23 (1H, ddd, J=9, 7.5, 7.5 Hz), 5.85 (1H, s), 6.95-7.12 (3H, m), 7.14-7.31 (2H, m), 7.54 (1H, d, J=9 Hz);

MASS (ES−): m/e 573;

MASS (ES+): m/e 575.

### EXAMPLE 36

Compound E36 was obtained from the Compound (99) in a manner similar to Example 1.

¹H-NMR (300 MHz, CDCl₃, δ): 0.83 (3H, t, J=7.3 Hz), 1.09 (3×3H, s), 1.22 (3H, d, J=7 Hz), 1.28 (3H, s), 1.38-1.52 (2H, m), 1.64 (1H, m), 1.70-1.91 (3H, m), 2.08-2.38 (6H, m), 2.94 (1H, dd, J=14, 6 Hz), 3.20 (1H, dd, J=14, 9.5 Hz), 3.28 (1H, m), 3.86 (1H, m), 4.22 (1H, m), 4.27 (1H, q, J=7 Hz), 4.67 (1H, m), 5.14 (1H, ddd, J=10, 9.5, 6 Hz), 5.87 (1H, s), 6.62 (1H, d, J=16 Hz), 6.86 (1H, dt, J=16.7 Hz), 7.08 (1H, d, J=10 Hz), 7.16 (2×1H, d, J=8.5 Hz), 7.24 (2×1H, d, J=8.5 Hz), 7.31-7.48 (6H, m), 7.52-7.69 (5H, m);

MASS (ES+): m/e 813.

### EXAMPLE 37

Compound E37 was obtained from the Compound E36 in a manner similar to Example 3.

¹H-NMR (300 MHz, CDCl₃, δ): 0.83 (3H, t, J=7 Hz), 1.10 (3×3H, s), 1.18 (3H, d, J=6.5 Hz), 1.20-1.30 (4H, m), 1.28 (3H, s), 1.40-1.50 (2H, m), 1.60 (1H, m), 1.72-1.89 (3H, m), 2.08-2.38 (4H, m), 2.51 (2H, m), 2, 94 (1H, dd, J=14, 6 Hz), 3.20 (1H, dd, J=14, 10 Hz), 3.28 (1H, m), 3.84 (1H, m), 4.19 (1H, q, J=6.5 Hz), 4.19 (1H, m), 4.67 (1H, m), 5.14 (1H, ddd, J=10, 10, 6 Hz), 5.87 (1H, s), 7.03 (1H, d, J=10.5 Hz), 7.17 (2×1H, d, J=9 Hz), 7.24 (2×1H, d, J=9 Hz), 7.33-7.50 (6H, m), 7, 56-7.68 (5H, m);

MASS (ES+): m/e 815.

### EXAMPLE 38

Compound E38 was obtained from the Compound E37 in a manner similar to Example 6.

¹H-NMR (300 MHz, CDCl₃, δ): 0.83 (3H, t, J=7.3 Hz), 1.20-1.40 (4H, m), 1.28 (3H, s), 1.38 (3H, d, J=7 Hz), 1.55-1.70 (3H, m), 1.72-1.90 (3H, m), 2.08-2.58 (6H, m), 2.94 (1H, dd, J=14.6 Hz), 3.20 (1H, dd, J=14, 10 Hz), 3.28 (1H, m), 3.56 (1H, d, J=5 Hz), 3.85 (1H, m), 4.15-4.30 (2H, m), 4.68 (1H, m), 5.14 (1H, ddd, J=10, 10, 6 Hz), 5.90 (1H, s), 7.06 (1H, d, J=10 Hz), 7.17 (2×1H, d, J=9 Hz), 7.24 (2×1H, d, J=9 Hz), 7, 58 (1H, d, J=10 Hz);

MASS (ES+): m/e 577;

[α]_{D}²⁵=−116.1° (c=0.31, CHCl₃).

### EXAMPLE 39

Compound E39 was obtained from the Compound (102) in a manner similar to Example 1.

¹H-NMR (300 MHz, CDCl₃, δ): 0.77 (3H, t, J=7.7 Hz), 0.91 (3H, t, J=7.3 Hz), 1.09 (9H, s), 1.22 (3H, d, J=7.0 Hz), 1.37-1.70 (4H, m), 1.71-1.92 (4H, m), 2.07-2.45 (6H, m), 2.97 (1H, dd, J=13.5, 5.8 Hz), 3.18-3.31 (2H, m), 3.83-3.95 (1H, m), 4.15-4.29 (1H, m), 4.27 (1H, q, J=6.9 Hz), 4.66 (1H, brd, J=6.9 Hz), 5.12-5.24 (1H, m), 5.79 (1H, s), 6.61 (1H, d, J=15.6 Hz), 6.86 (1H, dt, J=15.6, 6.7 Hz), 7.13 (1H, d, J=9.9 Hz), 7.17-7.29 (5H, m), 7.30-7.45 (6H, m), 7.49 (1H, d, J=10.6 Hz); 7.56-7.69 (4H, m);

MASS (ES+): m/e 793.32 (M+1).

### EXAMPLE 40

Compound E40 was obtained from the Compound E39 in a manner similar to Example 16.

¹H-NMR (300 MHz, CDCl₃, δ): 0.77 (3H, t, J=7.3 Hz), 0.92 (3H, t, J=7.3 Hz), 1.11 (9H, s), 1.15-1.35 (4H, m), 1.19 (3H, t, J=6.6 Hz), 1.37-1.69 (5H, m), 1.70-1.91 (3H, m), 2.11-2.46 (4H, m), 2.52 (2H, dt, J=7.0, 2.5 Hz), 2.97 (1H, dd, J=13.5, 6.3 Hz), 3.18-3.31 (2H, m), 3.82-3.96 (1H, m), 4.16-4.26 (1H, m), 4.19 (1H, q, J=6.5 Hz), 4.67 (1H, d, J=5.9 Hz), 5.12-5.24 (1H, m), 5.79 (1H, s), 7.08 (1H, d, J=10.6 Hz), 7.17-7.32 (5H, m), 7.33-7.49 (6H, m), 7.53 (1H, d, J=10.5 Hz), 7.58-7.69 (4H, m);

MASS (ES+): m/e 795.09 (M+1).

### EXAMPLE 41

Compound E41 was obtained from the Compound E40 in a manner similar to Example 6.

¹H-NMR (300 MHz, CDCl₃, δ): 0.77 (3H, t, J=6.9 Hz), 0.91 (3H, t, J=7.3 Hz), 1.21-1.41 (4H, m), 1.38 (3H, d, J=7.0 Hz), 1.51-1.70 (4H, m), 1.70-1.92 (4H, m), 2.08-2.58 (6H, m), 2.96 (1H, dd, J=13.6, 6.4 Hz), 3.16-3.30 (2H, m), 3.56 (1H, d, J=4.6 Hz), 3.82-3.94 (1H, m), 4.13-4.29 (2H, m), 4.67 (1H, brd, J=6.2 Hz), 5.11-5.24 (1H, m), 5.81 (1H, s), 7.11 (1H, d, J=10.3 Hz), 7.16-7.34 (5H, m), 7.50 (1H, d, J=10.4 Hz);

MASS (ES+): m/e 557.29 (M+1).

### EXAMPLE 42

Compound E42 was obtained from the Compound (105) in a manner similar to Example 1.

¹H-NMR (300 MHz, CDCl₃, δ): 0.83 (3H, t, J=7.5 Hz), 1.09 (3×3H, s), 1.22 (3H, d, J=7 Hz), 1.28 (3H, s), 1.45 (2H, m), 1.56-1.90 (4H, m), 2.07-2.40 (6H, m), 2.97 (1H, dd, J=13.5, 6.5 Hz), 3.24 (1H, dd, J=13.5, 9 Hz), 3.27 (1H, m), 3.87 (1H, m), 4.21 (1H, m), 4.27 (1H, q, J=7 Hz), 4.64 (1H, m), 5.19 (1H, ddd, J=10, 9, 6.5 Hz), 5.81 (1H, s), 6.62 (1H, brd, J=16 Hz), 6.87 (1H, dt, J=16, 7 Hz), 7.13 (1H, d, J=10 Hz), 7.17-7.49 (11H, m), 7.53 (1H, d, J=10 Hz), 7.56-7.76 (4H, m);

MASS (ES−): m/e 777.

### EXAMPLE 43

Compound E43 was obtained from the Compound (105) in a manner similar to Example 2.

¹H-NMR (300 MHz, CDCl₃, δ): 0.82 (3H, t, J=7.3 Hz), 1.09 (3×3H, s), 1.23 (3H, d, J=7 Hz), 1.28 (3H, s), 1.45 (2H, m), 1.5.8-1.92 (4H, m), 2.08-2.40 (6H, m), 2.97 (1H, dd, J=13.5, 6 Hz), 3.24 (1H, dd, J=13.5, 9.5 Hz), 3.27 (1H, m), 3.87 (1H, m), 4.21 (1H, dt, J=10, 7.5 Hz), 4.27 (1H, q, J=7 Hz), 4.67 (1H, dd, J=8, 2.5 Hz), 5.19 (1H, ddd, J=10, 9.5, 6 Hz), 5.81 (1H, s), 6.61 (1H, brd, J=16 Hz), 6.87 (1H, dt, J=16, 7 Hz), 7.13 (1H, d, J=10.5 Hz), 7.16-7.49 (11H, m), 7.53 (1H, d, J=10 Hz), 7.56-7.69 (4H, m);

MASS (ES−): m/e 777.

### EXAMPLE 44

Compound E44 was obtained from the Compound (105) in a manner similar to Example 29.

¹H-NMR (300 MHz, CDCl₃, δ): 0.80 (3H, t, J=7.4 Hz), 0.83 (3H, t, J=7.4 Hz), 1.10 (3×3H, s), 1.28 (3H, s), 1.44 (2H, m), 1.54-1.90 (6H, m), 2.08-2.40 (6H, m), 2.97 (1H, dd, J=14, 6 Hz), 3.24 (1H, dd, J=14, 9.5 Hz), 3.27 (1H, m), 3.87 (1H, m), 4.15 (1H, t, J=6 Hz), 4.20 (1H, m), 4.67 (1H, m), 5.19 (1H, ddd, J=10, 9.5, 6 Hz), 5.78 (1H, s), 6.55 (1H, d, J=16 Hz), 6.80 (1H, dt, J=16, 7 Hz), 7.12 (1H, d, J=10.5 Hz), 7.16-7.47 (11H, m), 7.53 (1H, d, J=10 Hz), 7.53-7.68 (4H, m);

MASS (ES−): m/e 791.

### EXAMPLE 45

Compound E45 was obtained from the Compound E42 in a manner similar to Example 16.

¹H-NMR (300 MHz, CDCl₃, δ): 0.83 (3H, t, J=7.3 Hz), 1.10 (3×3H, s), 1.18 (3H, d, J=7 Hz), 1.20-1.33 (4H, m), 1.28 (3H, s), 1.45 (2H, m), 1.60 (1H, m), 1.71-1.90 (3H, m), 2.08-2.40 (4H, m), 2.51 (2H, m), 2.97 (1H, dd, J=13.5, 6 Hz), 3.24 (1H, dd, J=13.5, 9 Hz), 3.27 (1H, m), 3.86 (1H, m), 4.18 (1H, m), 4.18 (1H, q, J=7 Hz), 4.67 (1H, m), 5.18 (1H, ddd, J=10, 9, 6 Hz), 5.81 (1H, s), 7.07 (1H, d, J=10.5 Hz), 7.16-7.31 (5H, m), 7.33-7.48 (6H, m), 7.57 (1H, d, J=10 Hz), 7.58-7.74 (4H, m);

MASS (ES−): m/e 779.

### EXAMPLE 46

Compound E46 was obtained from the Compound E43 in a manner similar to Example 16.

¹H-NMR (300 MHz, CDCl₃, δ): 0.83 (3H, t, J=7.3 Hz), 1.10 (3×3H, s), 1.16-1.33 (4H, m), 1.18 (3H, d, J=7 Hz), 1.28 (3H, s), 1.46 (2H, m), 1.58 (1H, m), 1.68-1.88 (3H, m), 2.07-2.40 (4H, m), 2.51 (2H, t, J=7 Hz), 2.97 (1H, dd, J=13.5, 6 Hz), 3.24 (1H, dd, J=13.5, 9.5 Hz), 3.27 (1H, m), 3.86 (1H, m), 4.18 (1H, m), 4.18 (1H, q, J=7 Hz), 4.67 (1H, dd, J=8, 2.5 Hz), 5.18 (1H, ddd, J=10, 9.5, 6 Hz), 5.82 (1H, s), 7.08 (1H, d J=10 Hz), 7.16-7.32 (5H, m), 7.33-7.50 (6H, m), 7.58 (1H, d, J=10 Hz), 7.58-7.70 (5H, m);

MASS (ES−): m/e 779.

### EXAMPLE 47

Compound E47 was obtained from the Compound E44 in a manner similar to Example 16.

¹H-NMR (300 MHz, CDCl₃, δ): 0.81 (3H, t, J=7 Hz), 0.83 (3H, t, J=7 Hz), 1.11 (9H, s), 1.15-1.26 (4H, m), 1.28 (3H, s), 1.30-1.46 (2H, m), 1.50-1.85 (6H, m), 2.07-2.48 (6H, m), 2.97 (1H, dd, J=14, 6 Hz), 3.24 (1H, dd, J=14, 9 Hz), 3.26 (1H, m), 3.86 (1H, m), 4.10-4.23 (2H, m), 4.67 (1H, m), 5.19 (1H, m), 5.80 (1H, s), 7.06 (1H, d, J=10.5 Hz), 7.16-7.31 (5H, m), 7.32-7.47 (6H, m), 7.54-7.66 (5H, m); MASS: (ES+) m/e 795.

### EXAMPLE 48

Compound E48 was obtained from the Compound E44 in a manner similar to Example 6 except that pyridine hydrofluoride was used instead of tetrabutylammonium fluoride.

¹H-NMR (300 MHz, CDCl₃, δ): 0.83 (3H, t, J=7 Hz), 0.94 (3H, t, J=7 Hz), 1.20-1.97 (8H, m), 1.29 (3H, s), 2.08-2.40 (6H, m), 2.97 (1H, dd, J=14, 6 Hz), 3.23 (1H, dd, J=14, 9 Hz), 3.26 (1H, m), 3.59 (1H, d, J=5 Hz), 3.87 (1H, m), 4.22 (1H, m), 4.67 (1H, m), 5.19 (1H, ddd, J=10, 9, 6 Hz), 5.84 (1H, s), 6.26 (1H, d, J=16 Hz), 7.00 (1H, dt, J=16, 7 Hz), 7.16 (1H, d, J=10 Hz), 7.19-7.32 (5H, m), 7.50 (1H, d, J=10 Hz);

MASS: (ES−) m/e 553.

### EXAMPLE 49

Compound E49 was obtained from the Compound E47 in a manner similar to Example 48.

¹H-NMR (300 MHz, CDCl₃, δ): 0.83 (3H, t, J=7 Hz), 0.94 (3H, t, J=7 Hz), 1.22-1.40 (4H, m), 1.28 (3H, s), 1.52-1.70 (4H, m), 1.71-1.98 (4H, m), 2.08-2.24 (2H, m), 2.25-2.40 (2H, m), 2.45 (2H, m), 2.96 (1H, ddd, J=13, 6, 5 Hz), 3.18-3.32 (2H, m), 3.50 (1H, d, J=5 Hz), 3.86 (1H, m), 4.14 (1H, m), 4.20 (1H, m), 4.67 (1H, m), 5.19 (1H, ddd, J=10, 9, 6 Hz), 5.82 (1H, s), 7.10 (1H, d, J=10 Hz), 7.16-7.32 (5H, m), 7.54 (1H, d, J=10 Hz);

MASS (ES+): m/e 557.

### EXAMPLE 50

Compound 50 was obtained from the Compound E42 in a manner similar to Example 48.

¹H-NMR (300 MHz, CDCl₃, δ): 0.83 (3H, t, J=7.3 Hz), 1.29 (3H, s), 1.38 (3H, d, J=7 Hz), 1.42-1.93 (6H, m), 2.07-2.40 (6H, m), 2.97 (1H, dd, J=13.5, 6 Hz), 3.24 (1H, dd, J=13.5, 9.5 Hz), 3.26 (1H, m), 3.65 (1H, d, J=5 Hz), 3.87 (1H, m), 4.22 (1H, dt, J=10.5, 7.5 Hz), 4.44 (1H, dq, J=7, 5 Hz), 4.67 (1H, dd, J=8, 2.5 Hz), 5.19 (1H, ddd, J=10, 9.5, 6 Hz), 5.84 (1H, s), 6.24 (1H, brd, J=16 Hz), 7.01 (1H, dt, J=16, 7 Hz), 7.16 (1H, d, J=10.5 Hz), 7.16-7.32 (5H, m), 7.50 (1H, d, J=10 Hz);

MASS (ES−): m/e 539.

### EXAMPLE 51

Compound E51 was obtained from the Compound E45 in a manner similar to Example 48.

¹H-NMR (300 MHz, CDCl₃, δ): 0.83 (3H, t, J=7.3 Hz), 1.20-1.42 (4H, m), 1.28 (3H, s), 1.38 (3H, d, J=7 Hz), 1.54-1.90 (6H, m), 2.08-2.58 (6H, m), 2.96 (1H, dd, J=13.5, 6 Hz), 3.24 (1H, dd, J=13.5, 9 Hz), 3.27 (1H, m), 3.57 (1H, d, J=4.5 Hz), 3.86 (1H, m), 4.14-4.29 (2H, m), 4.67 (1H, dd, J=8, 2.5 Hz), 5.19 (1H, ddd, J=10, 9, 6 Hz), 5.82 (1H, S), 7.10 (1H, d, J=10 Hz), 7.16-7.33 (5H, m), 7.55 (1H, d, J=10 Hz), 3.57 (1H, d, J=4.5 Hz);

MASS (ES−): m/e 541.

### EXAMPLE 52

Compound E52 was obtained from the Compound E46 in a manner similar to Example 48.

¹H-NMR (300 MHz, CDCl₃, δ): 0.83 (3H, t, J=7.5 Hz), 1.20-1.41 (4H, m), 1.28 (3H, s), 1.38 (3H, d, J=7 Hz), 1.52-1.90 (6H, m), 2.08-2.58 (6H, m), 2.96 (1H, dd, J=13.5, 6 Hz), 3.24 (1H, dd, J=13.5, 9.5 Hz), 3.27 (1H, m), 3.57 (1H, d, J=5 Hz), 3.86 (1H, m), 4.14-4.29 (2H, m), 4.67 (1H, dd, J=8, 2.5 Hz), 5.18 (1H, ddd, J=10, 9.5, 6 Hz), 5.83 (1H, s), 7.10 (1H, d, J=10 Hz), 7.16-7.31 (5H, m), 7.55 (1H, d, J=10 Hz);

MASS (ES−): m/e 541.

### EXAMPLE 53

To a solution of Compound E52 (7.7 mg) in pyridine (0.8 ml) was added (R)-(−)-α-methoxy-α-trifluoromethyl-α-phenylacetyl chloride (7.7 mg) at 0° C. and the mixture was stirred at ambient temperature until the Compound E52 was disappeared. The solvent was evaporated and the residue was purified by preparative thin layer chromatography (hexane/ethyl acetate=1:3 v/v) to give Compound E53 as an oil (8.4 mg).

¹H-NMR (300 MHz, CDCl₃, δ): 0.83 (3H, t, J=7.3 Hz), 1.20-1.38 (4H, m), 1.28 (3H, s), 1.44 (3H, d, J=7 Hz), 1.54-1.90 (6H, m), 2.08-2.59 (6H, m), 2.97 (1H, dd, J=13.5, 6 Hz), 3.24 (1H, dd, J=13.5, 9.5 Hz), 3.28 (1H, m), 3.63 (3H, s), 3.88 (1H, m), 4.14-4.25 (2H, m), 4.67 (1H, dd, J=8.5, 3 Hz), 5.19 (1H, ddd, J=10, 9.5, 6 Hz), 5.24 (1H, q, J=7 Hz), 5.81 (1H, s), 7.09 (1H, d, J=10 Hz), 7.16-7.32 (5H, m), 7.40-7.48 (3H, m), 7.56 (1H, d, J=10 Hz), 7.59-7.66 (2H, m);

MASS: (ES−) m/e 757.

### EXAMPLE 54

Compound E54 was obtained from the Compound E52 in a manner similar to Example 53 except that (S)-(−)-α-methoxy-α-trifluoromethyl-α-phenylacetyl chloride was used instead of (R)-(−)-α-methoxy-α-trifluoromethyl-α-phenylacetyl chloride.

¹H-NMR (300 MHz, CDCl₃, δ): 0.83 (3H, t, J=7.3 Hz), 1.18-1.38 (4H, m), 1, 28 (3H, s), 1, 46-1.87 (6H, m), 1.49 (3H, d, J=7 Hz), 2.09-2.48 (6H, m), 2.96 (1H, dd, J=13.5, 6 Hz), 3.24 (1H, dd, J=13.5, 9.5 Hz), 3.27 (1H, m), 3.58 (3H, s), 3.86 (1H, m), 4.12-4.26 (2H, m), 4.67 (1H, dd, J=8.2 Hz), 5.18 (1H, m), 5.28 (1H, q, J=7 Hz), 5.81 (1H, s), 7.08 (1H, d, J=10.5 Hz), 7.16-7.32 (5H, m), 7.40-7.47 (3H, m), 7.51-7.62 (3H, m);

MASS (ES−): m/e 757.

### EXAMPLE 55

Compound E55 was obtained from the Compound 51 in a manner similar to Example 45.

¹H-NMR (300 MHz, CDCl₃, δ): 0.83 (3H, t, J=7.5 Hz), 1.17-1.34 (4H, m), 1.28 (3H, s), 1.49 (3H, d, J=7 Hz), 1.51-1.63 (3H, m), 1.70-1.88 (3H, m), 2.08-2.50 (6H, m), 2.96 (1H, dd, J=13.5, 6.5 Hz), 3.23 (1H, dd, J=13.5, 9.5 Hz), 3.27 (1H, m), 3.58 (3H, s), 3.86 (1H, m), 4.18 (1H, m), 4.67 (1H, m), 5.18 (1H, m), 5.29 (1H, q, J=7 Hz), 5.80 (1H, s), 7.08 (1H, d, J=10 Hz), 7.16-7.32 (5H, m), 7.40-7.47 (3H, m), 7.51-7.64 (3H, m);

MASS (ES−): m/e 757.

### EXAMPLE 56

Compound E56 was obtained from the Compound 51 in a manner similar to Example 46.

¹H-NMR (300 MHz, CDCl₃, δ): 0.83 (3H, t, J=7.4 Hz), 1.17-1.37 (4H, m), 1.28 (3H, s), 1.44 (3H, d, J=7 Hz), 1.52-1.68 (3H, m), 1.70-1.90 (3H, m), 2.08-2.59 (6H, m), 2.97 (1H, dd, J=13.5, 6 Hz), 3.24 (1H, dd, J=13.5, 10 Hz), 3.27 (1H, m), 3.63 (3H, s), 3.86 (1H, m), 4.19 (1H, dt, J=10, 7.5 Hz), 4.67 (1H, dd, J=8, 2 Hz), 5.18 (1H, ddd, J=10, 10, 6 Hz), 5.25 (1H, q, J=7 Hz), 5.81 (1H, s), 7.09 (1H, d, J=10 Hz), 7.16-7.32 (5H, m), 7.40-7.48 (3H, m), 7.52-7.66 (2H, m), 7.56 (1H, d, J=10 Hz);

MASS (ES−): m/e 757.

### EXAMPLE 57

Compound E57 was obtained in a manner similar to Example 1.

¹H-NMR (300 MHz, CDCl₃, δ): 0.89 (3H, t, J=7.0 Hz), 0.96 (3H, t, J=6.5 Hz), 1.09 (9H, s), 1.17-1.89 (12H, m), 1.23 (3H, d, J=6.9 Hz), 1.99-2.44 (6H, m), 2.98 (1H, dd, J=13.5, 6.5 Hz), 3.20-3.32 (1H, m), 3.23 (1H, dd, J=13.5, 9.5 Hz), 3.80-3.93 (1H, m), 4.12-4.27 (1H, m), 4.27 (1H, q, J=7.0 Hz), 4.67 (1H, brd, J=5.5 Hz), 5.10-5.23 (1H, m), 5.79 (1H, s), 6.61 (1H, d, J=15.8 Hz), 6.87 (1H, dt, J=15.8, 6.7 Hz), 7.12 (1H, d, J=10.3 Hz), 7.16-7.29 (5H, m), 7.29-7.45 (6H, m), 7.48 (1H, d, J=11.0 Hz), 7.55-7.74 (4H, m);

MASS (ES+): m/e 821.39 (M+1).

### EXAMPLE 58

Compound E58 was obtained from the Compound E57 in a manner similar to Example 16.

¹H-NMR (300 MHz, CDCl₃, δ): 0.89 (3H, t, J=6.9 Hz), 0.97 (3H, t, J=7.0 Hz), 1.11 (9H, s), 1.16-1.67 (12H, m), 1.19 (3H, d, J=7.0 Hz), 1.68-1.88 (4H, m), 2.00-2.45 (4H, m), 2.51 (2H, brt, J=6.9 Hz), 2.98 (1H, dd, J=13.1, 6.3 Hz), 3.21-3.32 (1H, m), 3.23 (1H, dd, J=13.1, 9.2 Hz), 3.81-3.92 (1H, m), 4.13 (1H, q, J=7.1 Hz), 4.15-4.23 (1H, m), 4.68 (1H, brd, J=5.7 Hz), 5.10-5.22 (1H, m), 5.80 (1H, s), 7.07 (1H, d, J=10.3 Hz), 7.16-7.31 (6H, m), 7.33-7.48 (5H, m), 7.52 (1H, d, J=10.2 Hz), 7.58-7.75 (4H, m);

MASS (ES+): m/e 823.31 (M+1).

### EXAMPLE 59

Compound E59 was obtained from the Compound E57 in a manner similar to Example 6.

¹H-NMR (300 MHz, CDCl₃, δ): 0.89 (3H, t, J=6.5 Hz), 0.96 (3H, t, J=6.9 Hz), 1.12-1.41 (7H, m), 1.38 (3H, d, J=7.4 Hz), 1.41-1.69 (5H, m), 1.70-1.88 (4H, m), 2.00-2.58 (6H, m), 2.98 (1H, dd, J=12.5, 6.2 Hz), 3.19-3.31 (1H, m), 4.12-4.29 (1H, dd, J=12.5, 9.0 Hz), 3.55 (1H, d, J=4.8 Hz), 3.80-3.93 (1H, m), 4.12-4.29 (2H, m), 4.67 (1H, brd, J=5.4 Hz), 5.10-5.22 (1H, m), 5.81 (1H, s), 7.10 (1H, d, J=9.9 Hz), 7.16-7.32 (5H, m), 7.49 (1H, d, J=10.5 Hz);

MASS (ES+): m/e 585.34 (M+1).

### EXAMPLE 60

Compound E60 was obtained in a manner similar to Example 3.

### EXAMPLE 61

A solution of the Compound E60 (88 mg) in methanol (3 ml) was hydrogenated in the presence of palladium hydroxide, 20 wt % Pd (dry basis) on carbon (Pearlman's catalyst) (30 mg) for 2 hours. The catalyst was filtered off and the filtrate was concentrated in vacuo. The residue was purified by preparative thin layer chromatography (eluting with chloroform:methanol=20:1 v/v) to give Compound E61 as an amorphous (76 mg).

¹H-NMR (300 MHz, CDCl₃, δ): 1.04 (3×3H, S), 1.22-1.43 (4H, m), 1.38 (3H, d, J=7 Hz), 1.56-1.93 (6H, m), 2.17 (1H, m), 2.26-2.58 (3H, m), 2.91 (1H, dd, J=13, 5 Hz), 3.02 (1H, m), 3.19 (1H, dd, J=13, 11 Hz), 3.57 (1H, d, J=5 Hz), 3.91 (1H, m), 4.13 (1H, d, J=10.5 Hz), 4.24 (1H, dq, J=7, 5 Hz), 4.33 (1H, dt, J=10, 7.5 Hz), 4.60 (1H, m), 5.02 (1H, ddd, J=11, 10, 5 Hz), 6.23 (1H, d, J=10.5 Hz), 6.25 (1H, d, J=10 Hz), 7.12-7.32 (6H, m);

MASS: (ES+): m/e 557.

### EXAMPLE 62

Compound E62 was obtained in a manner similar to Example 1.

¹H-NMR (300 MHz, CDCl₃, δ): 0.71 (3H, d, J=6.9 Hz), 0.87 (3H, d, J=6.6 Hz), 1.09 (9H, s), 1.15 (3H, s), 1.36-1.92 (10H, m), 2.13-2.37 (3H, m), 2.99 (1H, dd, J=13.9, 7.0 Hz), 3.21 (1H, dd, J=13.9, 8.8 Hz), 3.26-3.36 (2H, m), 3.83-3.93 (1H, m), 4.17-4.31 (2H, m), 4.66-4.72 (1H, m), 5.21 (1H, ddd, J=10.6, 8.8, 7.0 Hz), 5.78 (1H, s), 6.61 (1H, d, J=15.8 Hz), 6.87 (1H, dt, J=15.8, 6.6 Hz), 7.13 (1H, d, J=10.6 Hz), 7.16-7.50 (10H, m), 7.54-7.74 (6H, m);

MASS: (ES+): m/e 793.32 (M+1).

### EXAMPLE 63

Compound E63 was obtained in a manner similar to Example 4.

¹H-NMR (300 MHz, CDCl₃, δ): 0.70 (3H, d, J=7.0 Hz), 0.87 (3H, d, J=6.6 Hz), 1.10 (9H, s), 1.15 (3H, s), 1.18 (3H, d, J=6.6 Hz), 1.21-1.88 (11H, m), 2.14-2.37 (2H, m), 2.51 (2H, dt, J=7.3, 2.2 Hz), 2.99 (1H, dd, J=13.9, 7.0 Hz), 3.20 (1H, dd, J=13.9, 8.8 Hz), 3.26-3.37 (1H, m), 3.82-3.92 (1H, m), 4.13-4.27 (2H, m), 4.66-4.71 (1H, m), 5.20 (1H, ddd, J=10.3, 8.8, 7.0 Hz), 5.77 (1H, s), 7.07 (1H, d, J=10.3 Hz), 7.16-7.31 (5H, m), 7.33-7.48 (5H, m), 7.58-7.74 (6H, m);

MASS: (ES+): m/e 795.29 (M+1).

### EXAMPLE 64

Compound E64 was obtained in a manner similar to Example 6.

¹H-NMR (300 MHz, CDCl₃, δ): 0.71 (3H, d, J=7.0 Hz), 0.88 (3H, d, J=6.6 Hz), 1.15 (3H, s), 1.21-1.43 (4H, m), 1.38 (3H, d, J=7.0 Hz), 1.52-1.72 (3H, m), 1.72-1.91 (3H, m), 2.11-2.57 (4H, m), 2.99 (1H, dd, J=13.6, 7.0 Hz), 3.20 (1H, dd, J=13.6, 8.8 Hz), 3.26-3.38 (2H, m), 3.57 (1H, brs), 3.83-3.93 (1H, m), 4.16-4.28 (2H, m), 4.66-4.73 (1H, m), 5.15-5.26 (1H, m), 5.85 (1H, s), 7.12 (1H, d, J=10.3 Hz), 7.16-7.32 (5H, m), 7.61 (1H, d, J=10.3 Hz);

MASS: (ES+): m/e 557.39 (M+1).

### EXAMPLE 65

Compound E65 was obtained in a manner similar to Example 1.

¹H-NMR (300 MHz, CDCl₃, δ): 1.08 (9H, s), 1.21 (3H, d, J=7.0 Hz), 1.32-1.84 (7H, m), 2.10-2.39 (3H, m), 2.85 (1H, dd, J=13.6, 10.6 Hz), 3.00 (1H, dd, J=14.3, 7.0 Hz), 3.04-3.15 (1H, m), 3.18 (1H, dd, J=13.6, 10.6 Hz), 3.39 (1H, dd, J=14.3, 8.4 Hz), 3.91-4.01 (1H, m), 4.21-4.32 (1H, m), 4.26 (1H, q, J=7.0 Hz), 4.59-4.64 (1H, m), 4.81-4.91 (1H, m), 5.06 (1H, dt, J=10.6, 5.1 Hz), 6.32 (1H, d, J=9.9 Hz), 6.45 (1H, d, J=10.6 Hz), 6.57 (1H, d, J=15.8 Hz), 6.82 (1H, dt, J=15.8, 7.0 Hz), 7.13-7.27 (5H, m), 7.29-7.50 (10H, m), 7.55-7.68 (5H, m), 7.74-7.83 (3H, m);

MASS: (ES−): m/e 875.40 (M−1).

### EXAMPLE 66

Compound E66 was obtained in a manner similar to Example 4.

¹H-NMR (300 MHz, CDCl₃, δ): 1.10 (9H, s), 1.13-1.26 (4H, m), 1.18 (3H, d, J=7.0 Hz), 1.34-1.46 (2H, m), 1.54-1.81 (4H, m), 2.15-2.41 (2H, m), 2.46 (2H, dt, J=7.3, 1.8 Hz), 2.85 (1H, dd, J=13.2, 5.1 Hz), 3.00 (1H, dd, J=13.9, 7.3 Hz), 3.04-3.15 (1H, m), 3.18 (1H, dd, J=13.2, 10.6 Hz), 3.39 (1H, dd, J=13.9, 8.4 Hz), 3.90-4.00 (1H, m), 4.17 (1H, q, J=7.0 Hz), 4.18-4.29 (1H, m), 4.58-4.64 (1H, m), 4.81-4, 91 (1H, m), 5.06 (1H, dt, J=10.6, 5.1 Hz), 6.30 (1H, d, J=9.9 Hz), 6.46 (1H, d, J=10.6 Hz), 7.09-7.27 (5H, m), 7.31-7.48 (10H, m), 7.58-7.68 (5H, m), 7.74-7.82 (3H, m);

MASS: (ES+): m/e 879.31 (M+1).

### EXAMPLE 67

Compound E67 was obtained in a manner similar to Example 6.

¹H-NMR (300 MHz, CDCl₃, δ): 1.16-1.40 (4H, m), 1.36 (3H, d, J=7.0 Hz), 1.47-1.87 (6H, m), 2.14-2.51 (4H, m), 2.86 (1H, dd, J=13.6, 5.5 Hz), 3.02 (1H, dd, J=14.3, 7.3 Hz), 3.06-3.14 (1H, m), 3.19 (1H, dd, J=13.6, 10.6 Hz), 3.39 (1H, dd, J=14.3, 8.4 Hz), 3.56 (1H, br), 3.91-4.01 (1H, m), 4.16-4.31 (2H, m), 4.59-4.66 (1H, m), 4.81-4.92 (1H, m), 5.08 (1H, dt, J=10.6, 5.5 Hz), 6.32 (1H, d, J=9.9 Hz), 6.47 (1H, d, J=10.6 Hz), 7.11-7.30 (6H, m), 7.35 (1H, dd, J=8.4, 1.5 Hz), 7.41-7.51 (2H, m), 7.67 (1H, s), 7.74-7.84 (3H, m);

MASS: (ES+): m/e 641.32 (M+1).

### EXAMPLE 68

Compound E68 was obtained in a manner similar to Example 1.

¹H-NMR (300 MHz, CDCl₃, δ): 1.08 (9H, s), 1.22 (3H, d, J=7.0 Hz), 1.30-1.85 (7H, m), 2.11-2.26 (2H, m), 2.29-2.38 (1H, m), 2.86 (1H, d, J=16.5 Hz), 2.94 (1H, dd, J=13.2, 5.3 Hz), 3.11-3.22 (1H, m), 3.31 (1H, dd, J=13.2, 10.3 Hz), 3.62 (1H, d, J=16.5 Hz), 3.90-4.02 (3H, m), 4.16-4.27 (1H, m), 4.27 (1H, q, J=7 Hz), 4.64-4.70 (1H, m), 5.15 (1H, dt, J=10.3, 5.3 Hz), 6.32 (1H, s), 6.58 (1H, d, J=15.8 Hz), 6.84 (1H, dt, J=15.8, 6.8 Hz), 7.15-7.29 (10H, m), 7.29-7.46 (6H, m), 7.50 (1H, d, J=10.3 Hz), 7.55-7.75 (4H, m);

MASS: (ES+): m/e 839.28 (M+1).

### EXAMPLE 69

Compound E69 was obtained in a manner similar to Example 4.

¹H-NMR (300 MHz, CDCl₃, δ): 1.10 (9H, s), 1.18 (3H, d, J=7.0 Hz), 1.15-1.35 (2H, m), 1.36-1.49 (1H, m), 1.54-1.84 (7H, m), 2.10-2.41 (2H, m), 2.49 (2H, dt, J=7.7, 2.6 Hz), 2.85 (1H, d, J=15.8 Hz), 2.93 (1H, dd, J=13.2, 5.1 Hz), 3.11-3.22 (1H, m), 3.30 (1H, dd, J=13.2, 10.3 Hz), 3.62 (1H, d, J=16.5 Hz), 3.89-3.99 (1H, m), 3.97 (1H, d, J=16.5 Hz), 3.98 (1H, d, J=15.8 Hz), 4.13-4.24 (1H, m), 4.15 (1H, q, J=7.0 Hz), 4.64-4.70 (1H, m), 5.14 (1H, dt, J=10.3, 5.1 Hz), 6.32 (1H, s), 7.12-7.31 (10H, m), 7.32-7.47 (6H, m), 7.53 (1H, d, J=10.3 Hz), 7.58-7.68 (4H, m);

MASS: (ES+): m/e 841.22 (M+1).

### EXAMPLE 70

Compound E70 was obtained in a manner similar to Example 6.

¹H-NMR (300 MHz, CDCl₃, δ): 1.37 (3H, d, J=7.0 Hz), 1.51-1.86 (9H, m), 2.06-2.26 (2H, m), 2.27-2.54 (3H, m), 2.86 (1H, d, J=16.2 Hz), 2.92 (1H, dd, J=13.2, 5.1 Hz), 3.09-3.21 (1H, m), 3.29 (1H, dd, J=13.2, 10.3 Hz), 3.55 (1H, d, J=4.5 Hz), 3.60 (1H, d, J=16.2 Hz), 3.88-4.02 (1H, m), 3.97 (2H, d, J=16.2 Hz), 4.13-4.27 (2H, m), 4.63-4.70 (1H, m), 5.14 (1H, dt, J=10.3, 5.1 Hz), 6.39 (1H, s), 7.13-7.31 (10H, m), 7.51 (1H, d, J=10.3 Hz);

MASS: (ES+): m/e 603.35 (M+1).

### EXAMPLE 71

Compound E71 was obtained in a manner similar to Example 1.

¹H-NMR (300 MHz, CDCl₃, δ): 0.83 (3H, t, J=7.0 Hz), 1.10 (9H, s), 1.23 (3H, d, J=7.0 Hz), 1.29 (3H, s), 1.39-1.91 (8H, m), 2.08-2.38 (4H, m), 3.13 (1H, dd, J=13.2, 6.2 Hz), 3.20-3.29 (1H, m), 3.42 (1H, dd, J=13.2, 9.9 Hz), 3.84-3.93 (1H, m), 4.17-4.27 (1H, m), 4.28 (1H, q, J=7.0 Hz), 4.62-4.68 (1H, m), 5.30 (1H, dt, J=9.9, 6.2 Hz), 5.87 (1H, s), 6.62 (1H, d, J=15.4 Hz), 6.88 (1H, dt, J=15.4, 6.6 Hz), 7.15 (1H, d, J=9.9 Hz), 7.31-7.49 (9H, m), 7.57-7.74 (6H, m), 7.74-7.83 (3H, m);

MASS: (ES+): m/e 829.43 (M+1).

### EXAMPLE 72

Compound E72 was obtained in a manner similar to Example 4.

¹H-NMR (300 MHz, CDCl₃, δ): 0.83 (3H, t, J=7.3 Hz), 1.00-1.34 (4H, m), 1.10 (9H, s), 1.19 (3H, d, J=6.6 Hz), 1.28 (3H, s), 1.35-1.89 (12H, m), 2.07-2.40 (4H, m), 2.51 (2H, dt, J=7.3, 2.2 Hz), 3.12 (1H, dd, J=13.6, 5.9 Hz), 3.18-3.30 (1H, m), 3.41 (1H, dd, J=13.6, 9.9 Hz), 3.81-3.92 (1H, m), 4.12-4.27 (3H, m), 4.61-4.67 (1H, m), 5.29 (1H, dt, J=9.9, 5.9 Hz), 5.83 (1H, s), 7.08 (1H, d, J=10.3 Hz), 7.32-7.49 (9H, m), 7.57-7.73 (6H, m), 7.73-7.83 (3H, m);

MASS: (ES+): m/e 831.35 (M+1).

### EXAMPLE 73

Compound E73 was obtained in a manner similar to Example 6.

¹H-NMR (300 MHz, CDCl₃, δ): 0.83 (3H, t, J=7.3 Hz), 1.20-1.40 (4H, m), 1.29 (3H, s), 1.38 (3H, d, J=7.0 Hz), 1.52-1.90 (6H, m), 2.07-2.57 (6H, m), 3.12 (1H, dd, J=13.6, 5.9 Hz), 3.19-3.29 (1H, m), 3.41 (1H, dd, J=13.6, 9.9 Hz), 3.56 (1H, d, J=4.8 Hz), 3.82-3.92 (1H, m), 4.15-4.29 (2H, m), 4.62-4.68 (1H, m), 5.30 (1H, dt, J=9.9, 5.9 Hz), 5.88 (1H, s), 7.11 (1H, d, J=10.3 Hz), 7.35-7.40 (1H, m), 7.40-7.49 (2H, m), 7.62 (1H, d, J=10.3 Hz), 7.69 (1H, s), 7.74-7.83 (3H, m);

MASS: (ES+): m/e 593.35 (M+1).

### EXAMPLE 74

Compound E74 was obtained in a manner similar to Example 1.

¹H-NMR (300 MHz, CDCl₃, δ): 1.10 (9H, s), 1.15-1.88 (5H, m), 1.19-1.29 (3H, m), 2.14-2.37 (4H, m), 2.86 (1H, dd, J=13.2, 5.1 Hz), 2.98 (1H, dd, J=14.7, 5.9 Hz), 3.05-3.16 (1H, m), 3.18 (1H, dd, J=13.2, 10.6 Hz), 3.35 (1H, dd, J=14.7, 8.8 Hz), 3.77 (3H, s), 3.93-4.02 (1H, m), 4.21-4.33 (3H, m), 4.59-4.64 (1H, m), 4.81 (1H, dt, J=9.5, 6.6 Hz), 5.08 (1H, dt, J=10.6, 5.1 Hz), 6.36 (1H, d, J=9.9 Hz), 6.45 (1H, d, J=10.6 Hz), 6.58 (1H, d, J=15.4 Hz), 6.84 (1H, dt, J=15.4, 7.0 Hz), 6.87 (1H, s), 7.08-7.26 (7H, m), 7.32-7.49 (7H, m), 7.56-7.68 (6H, m);

MASS (ES−) m/e 878.36 (M−1).

### EXAMPLE 75

Compound E75 was obtained in a manner similar to Example 4.

¹H-NMR (300 MHz, CDCl₃, δ): 1.10 (9H, s), 1.13-1.32 (3H, m), 1.38-1.50 (2H, m), 1.54-1.84 (5H, m), 2.16-2.38 (4H, m), 2.45-2.53 (2H, m), 2.86 (1H, dd, J=13.2, 5.1 Hz), 2.99 (1H, dd, J=14.7, 5.9 Hz), 3.05-3.16 (1H, m), 3.18 (1H, dd, J=13.2, 10.6 Hz), 3.35 (1H, dd, J=14.7, 9.5 Hz), 3.77 (3H, s), 3.92-4.01 (1H, m), 4.14-4.24 (1H, m), 4.26 (2H, q, J=7.0 Hz), 4.59-4.64 (1H, m), 4.82 (1H, dt, J=9.5, 5.9 Hz), 5.08 (1H, dt, J=10.6, 5.1 Hz), 6.34 (1H, d, J=9.9 Hz), 6.47 (1H, d, J=10.6 Hz), 6.87 (1H, s), 7.09-7.31 (7H, m), 7.33-7.49 (7H, m), 7.58-7.67 (6H, m);

MASS (ES+): m/e 882.37 (M+1).

### EXAMPLE 76

Compound E76 was obtained in a manner similar to Example 6.

¹H-NMR (300 MHz, CDCl₃, δ): 1.19-1.40 (4H, m), 1.38 (3H, d, J=7.0 Hz), 1.50-1.86 (6H, m), 2.13-2.55 (4H, m), 2.85 (1H, dd, J=13.6, 5.1 Hz), 2.98 (1H, dd, J=14.7, 6.6 Hz), 3.04-3.15 (1H, m), 3.17 (1H, dd, J=13.6, 10.6 Hz), 3.34 (1H, dd, J=14.7, 8.8 Hz), 3.52-3.59 (1H, m), 3.73 (3H, s), 3.92-4.01 (1H, m), 4.17-4.31 (2H, m), 4.58-4.65 (1H, m), 4.81 (1H, ddd, J=9.5, 8.8, 6.6 Hz), 5.08 (1H, dt, J=10.6, 5.1 Hz), 6.34 (1H, d, J=9.5 Hz), 6.46 (1H, d, J=10.6 Hz), 6.87 (1H, s), 7.08-7.31 (9H, m), 7.60 (1H, d, J=8.1 Hz);

MASS (ES+): m/e 644.48 (M+1);

### EXAMPLE 77

Compound E77 was obtained in a manner similar to Example 1.

¹H-NMR (300 MHz, CDCl₃, δ): 0.84 (3H, t, J=7.3 Hz), 1.09 (9H, s), 1.22 (3H, d, J=7.0 Hz), 1.28 (3H, s), 1.38-1.91 (7H, m), 2.08-2.39 (5H, m), 2.30 (3H, s), 2.91 (1H, dd, J=13.6, 6.2 Hz), 3.20 (1H, dd, J=13.6, 10.3 Hz), 3.23-3.33 (1H, m), 3.82-3.92 (1H, m), 4.16-4.25 (1H, m), 4.27 (1H, q, J=6.6 Hz), 4.64-4.70 (1H, m), 5.16 (1H, dt, J=10.3, 6.2 Hz), 5.84 (1H, s), 6.61 (1H, d, J=15.7 Hz), 6.87 (1H, dt, J=15.7, 6.6 Hz), 7.05-7.13 (4H, m), 7.14 (1H, d, J=9.5 Hz), 7.31-7.45 (6H, m), 7.51 (1H, d, J=10.3 Hz), 7.56-7.68 (4H, m);

MASS (ES+): m/e 793.57 (M+1).

### EXAMPLE 78

Compound E78 was obtained in a manner similar to Example 4.

¹H-NMR (300 MHz, CDCl₃, δ): 0.84 (3H, t, J=7.7 Hz), 1.10 (9H, s), 1.18 (3H, d, J=6.6 Hz), 1.28 (3H, s), 1.38-1.88 (9H, m), 2.07-2.40 (5H, m), 2.30 (3H, s), 2.51 (2H, dt, J=7.3, 2.6 Hz), 2.91 (1H, dd, J=13.2, 6.2 Hz), 3.20 (1H, dd, J=13.2, 9.9 Hz), 3.24-3.33 (1H, m), 3.81-3.91 (1H, m), 4.14-4.24 (1H, m), 4.18 (1H, q, J=6.6 Hz), 4.64-4.70 (1H, m), 5.16 (1H, dt, J=9.9, 6.2 Hz), 5.84 (1H, s), 7.05-7.15 (5H, m), 7.33-7.48 (6H, m), 7.55 (1H, d, J=9.9 Hz), 7.59-7.67 (4H, m);

MASS (ES+): m/e 795.52 (M+1).

### EXAMPLE 79

Compound E79 was obtained in a manner similar to Example 6.

¹H-NMR (300 MHz, CDCl₃, δ): 0.84 (3H, t, J=7.3 Hz), 1.20-1.41 (6H, m), 1.28 (3H, s), 1.38 (3H, d, J=7.0 Hz), 1.52-1.89 (4H, m), 2.07-2.40 (4H, m), 2.30 (3H, s), 2.46 (2H, dt, J=12.1, 7.3 Hz), 2.91 (1H, dd, J=13.7, 6.2 Hz), 3.20 (1H, dd, J=13.7, 9.9 Hz), 3.25-3.32 (1H, m), 3.55 (1H, d, J=4.8 Hz), 3.81-3.91 (1H, m), 4.14-4.28 (2H, m), 4.64-4.70 (1H, m), 5.16 (1H, dt, J=9.9, 6.2 Hz), 5.85 (1H, s), 7.05-7.15 (5H, m), 7.53 (1H, d, J=9.9 Hz);

MASS (ES+): m/e 557.41 (M+1).

### EXAMPLE 80

Compound E80 was obtained in a manner similar to Example 1.

¹H-NMR (300 MHz, CDCl₃, δ): 0.84 (3H, t, J=7.2 Hz), 1.09 (9H, s), 1.22 (3H, d, J=6.9 Hz), 1.29 (3H, s), 1.38-1.51 (1H, m), 1.56-1.71 (1H, m), 1.73-2.43 (H, m), 3.13 (1H, dd, J=15.0, 5.7 Hz), 3.52 (1H, dd, J=15.0, 9.9 Hz), 3.73-3.84 (1H, m), 3.87-3.98 (1H, m), 4.17-4.26 (1H, m), 4.27 (1H, q, J=6.9 Hz), 4.68 (1H, dd, J=7.5, 2.4 Hz), 5.57 (1H, dt, J=9.9, 5.7 Hz), 5.87 (1H, s), 6.60 (1H, d, J=15.6 Hz), 6.86 (1H, dt, J=15.6, 6.3 Hz), 7.08-7.14 (1H, m), 7.15-7.21 (2H, m), 7.30-7.52 (6H, m), 7.55-7.68 (6H, m), 8.43-8.48 (1H, m);

MASS (ES+): m/e 780.56 (M+1).

### EXAMPLE 81

Compound E81 was obtained in a manner similar to Example 4.

¹H-NMR (300 MHz, CDCl₃, δ): 0.80 (3H, t, J=7.2 Hz), 1.10 (9H, s), 1.18 (3H, d, J=6.6 Hz), 1.20-2.46 (14H, m), 1.26 (3H, s), 2.46-2.57 (2H, m), 3.18-3.32 (1H, m), 3.58-3.97 (3H, m), 4.14-4.26 (2H, m), 4.66-4.73 (1H, m), 5.53-5.63 (1H, m), 5.90 (1H, s), 7.04-7.14 (1H, m), 7.28-7.48 (8H, m), 7.54-7.86 (6H, m), 8.50-8.58 (1H, m);

MASS (ES+): m/e 782.57 (M+1).

### EXAMPLE 82

Compound E82 was obtained in a manner similar to Example 6.

¹H-NMR (300 MHz, CDCl₃, δ): 0.87 (3H, t, J=7.2 Hz), 1.17-1.96 (12H, m), 1.29 (3H, s), 1.38 (3H, d, J=6.9 Hz), 2.06-2.57 (4H, m), 3.11-3.24 (1H, m), 3.12 (1H, dd, J=15.0, 5.7 Hz), 3.52 (1H, dd, J=15.0, 10.2 Hz), 3.74-3.84 (1H, m), 3.88-3.98 (1H, m), 4.14-4.28 (2H, m), 4.68 (1H, dd, J=7.5, 2.4 Hz), 5.58 (1H, dt, J=10.2, 5.7 Hz), 5.92 (1H, s), 7.07-7.12 (1H, m), 7.14-7.20 (2H, m), 7.42-7.52 (1H, m), 7.57 (1H, dt, J=7.5, 1.8 Hz), 8.42-8.47 (1H, s);

MASS (ES+): m/e 544.49 (M+1).

### EXAMPLE 83

Compound E83 was obtained in a manner similar to Example 1.

¹H-NMR (300 MHz, CDCl₃, δ): 0.85 (3H, t, J=7.5 Hz), 1.09 (9H, s), 1.14 (3H, d, J=7.3 Hz), 1.29 (3H, s), 1.34-1.91 (6H, m), 2.00-2.40 (6H, m), 2.16 (3H, s), 2.92 (1H, dd, J=13.7, 6.0 Hz), 3.14-3.34 (2H, m), 3.78-3.94 (1H, m), 4.16-4.33 (2H, m), 4.67 (1H, brd, J=6.0 Hz); 5.08-5.24 (1H, m), 5.90 (1H, brs), 6.61 (1H, brd, J=15.8 Hz), 6.80-6.94 (1H, m), 7.05-7.24 (4H, m), 7.30-7.48 (7H, m), 7.50-7.71 (6H, m);

MASS (ES+): m/e 836.37 (M+1).

### EXAMPLE 84

Compound E84 was obtained in a manner similar to Example 4.

¹H-NMR (300 MHz, CDCl₃, δ): 0.84 (3H, t, J=7.3 Hz), 1.10 (9H, s), 1.13-1.88 (10H, m), 1.21 (3H, d, J=6.8 Hz), 1.28 (3H, s), 2.07-2.22 (2H, m), 2.16 (3H, s), 2.24-2.39 (2H, m), 2.44-2.56 (2H, m), 2.84-2.97 (1H, m), 3.12-3.34 (2H, m), 3.77-3.94 (1H, m), 4.10-4.34 (2H, m), 4.66 (1H, brd, J=6.6 Hz), 5.07-5.21 (1H, m), 5.88 (1H, brs), 7.06 (1H, d, J=10.6 Hz), 7.13 (1H, s), 7.18 (2H, d, J=8.1 Hz), 7.31-7.50 (7H, m), 7.53-7.71 (6H, m);

MASS (ES+): m/e 838.48 (M+1).

### EXAMPLE 85

Compound E85 was obtained in a manner similar to Example 6.

¹H-NMR (300 MHz, CDCl₃, δ): 0.85 (3H, t, J=7.3 Hz), 1.21-1.91 (10H, m), 1.29 (3H, S), 1.39 (3H, d, J=7.3 Hz), 2.08-2.24 (2H, m), 2.17 (3H, s), 2.26-2.40 (2H, m), 2.41-2.58 (2H, m), 2.91 (1H, dd, J=13.6, 5.5 Hz), 3.14-3.34 (2H, m), 3.51-3.61 (1H, m), 3.75-3.92 (1H, m), 4.13-4.30 (2H, m), 4.67 (1H, brd, J=6.2 Hz), 5.08-5.22 (1H, m), 5.90 (1H, s), 7.10 (1H, d, J=9.9 Hz), 7.16 (1H, s), 7.19 (2H, d, J=8.6 Hz), 7.40 (2H, d, J=8.6 Hz), 7.56 (1H, d, J=9.2 Hz);

MASS (ES+): m/e 600.42 (M+1).

### EXAMPLE 86

Compound E86 was obtained in a manner similar to Example 1.

¹H-NMR (300 MHz, CDCl₃, δ): 0.77 (3H, t, J=7.3 Hz), 1.09 (9H, s), 1.19-2.35 (14H, m), 1.22 (3H, d, J=6.6 Hz), 1.27 (3H, s), 2.93 (1H, dt, J=13.2, 2.9 Hz), 3.04 (1H, dd, J=13.9, 7.7 Hz), 3.21 (1H, dd, J=13.9, 7.9 Hz), 4.00 (1H, brd, J=12.5 Hz), 4.17-4.28 (1H, m), 4.27 (1H, q, J=6.6 Hz), 4.98-5.08 (1H, m), 5.36 (1H, dt, J=10.6, 7.9 Hz), 5.95 (1H, s), 6.49 (1H, d, J=10.3 Hz), 6.62 (1H, d, J=15.8 Hz), 6.85 (1H, dt, J=15.8, 7.0 Hz), 7.15-7.48 (11H, m), 7.51 (1H, d, J=10.6 Hz), 7.55-7.70 (4H, m);

MASS (ES+): m/e 793.52 (M+1).

### EXAMPLE 87

Compound E87 was obtained in a manner similar to Example 4.

¹H-NMR (300 MHz, CDCl₃, δ): 0.77 (3H, t, J=7.3 Hz), 1.03-1.65 (9H, m), 1.10 (9H, s), 1.18 (3H, d, J=7.0 Hz), 1.27 (3H, s), 1.68-1.84 (2H, m), 1.91-2.34 (5H, m), 2.51 (2H, dt, J=7.2, 1.8 Hz), 2.94 (1H, dt, J=13.6, 2.9 Hz), 3.04 (1H, dd, J=13.9, 7.1 Hz), 3.21 (1H, dd, J=13.9, 7.7 Hz), 3.99 (1H, brd, J=12.8 Hz), 4.13-4.26 (2H, m), 4.98-5.07 (1H, m), 5.36 (1H, dt, J=10.3, 7.5 Hz), 5.93 (1H, s), 6.45 (1H, d, J=10.3 Hz), 7.15-7.31 (5H, m), 7.32-7.49 (6H, m), 7.54 (1H, d, J=10.3 Hz), 7.58-7.71 (4H, m);

MASS (ES+): m/e 795.53 (M+1).

### EXAMPLE 88

Compound E88 was obtained in a manner similar to Example 6.

¹H-NMR (300 MHz, CDCl₃, δ): 0.77 (3H, t, J=7.3 Hz), 1.17-1.43 (4H, m), 1.27 (3H, s), 1.38 (3H, d, J=7.0 Hz), 1.45-1.69 (6H, m), 1.70-1.86 (1H, m), 1.90-2.17 (4H, m), 2.19-2.34 (1H, m), 2.35-2.58 (2H, m), 2.93 (1H, dt, J=13.2, 2.6 Hz), 3.03 (1H, dd, J=13.9, 7.3 Hz), 3.21 (1H, dd, J=13.9, 7.7 Hz), 3.58 (1H, d, J=4.8 Hz), 3.99 (1H, brd, J=12.8 Hz), 4.15-4.30 (2H, m), 5.00-5.06 (1H, m), 5.36 (1H, dt, J=10.3, 7.5 Hz), 6.02 (1H, s), 6.50 (1H, d, J=10.3 Hz), 7.15-7.33 (5H, m), 7.53 (1H, d, J=10.3 Hz);

MASS (ES+): m/e 557.39 (M+1).

### EXAMPLE 89

Compound E89 was obtained in a manner similar to Example 6.

¹H-NMR (300 MHz, CDCl₃, δ): 0.77 (3H, t, J=7.3 Hz), 1.18-2.38 (14H, m), 1.28 (3H, s), 1.38 (3H, d, J=7.3 Hz), 2.92 (1H, dt, J=13.2, 2.6 Hz), 3.04 (1H, dd, J=13.9, 7.3 Hz), 3.21 (1H, dd, J=13.9, 7.7 Hz), 3.66 (1H, d, J=5.1 Hz), 3.95-4.06 (1H, m), 4.17-4.31 (1H, m), 4.39-4.51 (1H, m), 5.03 (1H, brd, J=5.5 Hz), 5.36 (1H, dt, J=10.3, 7.7 Hz), 5.99 (1H, s), 6.24 (1H, d, J=15.8 Hz), 6.53 (1H, d, J=10.3 Hz), 7.00 (1H, dt, J=15.8, 7.0 Hz), 7.15-7.35 (5H, m), 7.48 (1H, d, J=10.3 Hz);

MASS (ES+): m/e 555.40 (M+1).

### EXAMPLE 90

Compound E90 was obtained in a manner similar to Example 1.

¹H-NMR (300 MHz, CDCl₃, δ): 0.83 (3H, t, J=7.3 Hz), 1.10 (9H, s), 1.21 (3H, d, J=7.0 Hz), 1.28 (3H, s), 1.38-2.42 (12H, m), 2.90-2.99 (1H, m), 2.99 (3H, s), 3.20 (1H, dd, J=13.6, 8.8 Hz), 3.26-3.36 (1H, m), 3.79-3.92 (1H, m), 4.17-4.32 (2H, m), 4.68 (1H, brd, J=8.1 Hz), 5.10-5.21 (1H, m), 5.85 (1H, s), 6.42 (1H, s), 6.62 (1H, brd, J=15.6 Hz), 6.87 (1H, dt, J=15.6, 6.6 Hz), 7.07 (1H, d, J=10.3 Hz), 7.13 (2H, d, J=8.4 Hz), 7.23 (2H, d, J=8.4 Hz), 7.31-7.69 (10H, m);

MASS (ES−) m/e 870.56 (M−1).

### EXAMPLE 91

Compound E91 was obtained in a manner similar to Example 4.

¹H-NMR (300 MHz, CDCl₃, δ): 0.82 (3H, t, J=7.3 Hz), 1.10 (9H, s), 1.21 (3H, d, J=7.0 Hz), 1.28 (3H, s), 1.37-1.66 (7H, m), 1.71-1.91 (3H, m), 2.07-2.39 (4H, m), 2.51 (2H, dt, J=7.0, 2.2 Hz), 2.95 (1H, dd, J=13.6, 6.6 Hz), 2.99 (3H, s), 3.20 (1H, dd, J=13.6, 9.2 Hz), 3.26-3.36 (1H, m), 3.79-3.91 (1H, m), 4.14-4.24 (1H, m), 4.25 (1H, q, J=7.0 Hz), 4.69 (1H, brd, J=7.0 Hz), 5.09-5.21 (1H, m), 5.90 (1H, s), 6.46 (1H, s), 7.03 (1H, d, J=9.9 Hz), 7.12 (2H, d, J=8.4 Hz), 7.23 (2H, d, J=8.4 Hz), 7.32-7.50 (6H, m), 7.57-7.70 (5H, m);

MASS (ES−) m/e 872.46 (M−1).

### EXAMPLE 92

Compound E92 was obtained in a manner similar to Example 6.

¹H-NMR (300 MHz, CDCl₃, δ): 0.83 (3H, t, J=7.3 Hz), 1.21-1.41 (4H, m), 1.29 (3H, s), 1.38 (3H, d, J=7.0 Hz), 1.51-1.69 (3H, m), 1.70-1.90 (3H, m), 2.08-2.58 (6H, m), 2.95 (1H, dd, J=13.9, 7.0 Hz), 2.99 (3H, s), 3.20 (1H, dd, J=13.9, 9.5 Hz), 3.26-3.37 (1H, m), 3.55 (1H, brd, J=4.0 Hz), 3.79-3.91 (1H, m), 4.15-4.29 (2H, m), 4.69 (1H, brd, J=7.3 Hz), 5.15 (1H, dt, J=9.6, 6.6 Hz), 5.94 (1H, s), 6.56 (1H, s), 7.06 (1H, d, J=10.3 Hz), 7.13 (2H, d, J=8.4 Hz), 7.22 (2H, d, J=8.4 Hz), 7.60 (2H, d, J=10.3 Hz);

MASS (ES+): m/e 636.51 (M+1).

### EXAMPLE 93

Compound E93 was obtained in a manner similar to Example 1.

¹H-NMR (300 MHz, CDCl₃, δ): 0.84 (3H, t, J=7.3 Hz), 1.10 (9H, s), 1.23 (3H, d, J=6.6 Hz), 1.30 (3H, s), 1.36-1.93 (6H, m), 2.08-2.41 (6H, m), 3.02 (1H, dd, J=13.5, 6.2 Hz), 3.22-3.38 (2H, m), 3.82-3.96 (1H, m), 4.15-4.28 (1H, m), 4.27 (1H, q, J=6.6 Hz), 4.69 (1H, brd, J=6.0 Hz), 5.17-5.30 (1H, m), 5.85 (1H, s), 6.62 (1H, d, J=15.3 Hz), 6.87 (1H, dt, J=15.3, 7.0 Hz), 7.13 (1H, d, J=10.3 Hz), 7.27-7.48 (11H, m), 7.49-7.69 (9H, m);

MASS (ES+): m/e 855.61 (M+1).

### EXAMPLE 94

Compound E94 was obtained in a manner similar to Example 4.

¹H-NMR (300 MHz, CDCl₃, δ): 0.84 (3H, t, J=7.4 Hz), 1.10 (9H, s), 1.19 (3H, d, J=6.9 Hz), 1.20-1.34 (7H, m), 1.29 (3H, s), 1.39-1.60 (3H, m), 1.69-1.90 (3H, m), 2.08-2.40 (4H, m), 2.52 (2H, dt, J=7.3, 2.2 Hz), 3.02 (1H, dd, J=13.5, 6.3 Hz), 3.20-3.38 (2H, m), 3.82-3.94 (1H, m), 4.12-4.26 (2H, m), 4.69 (1H, brd, J=5.7 Hz), 5.15-5.29 (1H, m), 5.84 (1H, s), 7.07 (1H, d, J=10.3 Hz), 7.27-7.47 (12H, m), 7.48-7.69 (8H, m);

MASS (ES+): m/e 857.66 (M+1).

### EXAMPLE 95

Compound E95 was obtained in a manner similar to Example 6.

¹H-NMR (300 MHz, CDCl₃, δ): 0.84 (3H, t, J=7.5 Hz), 1.21-1.41 (4H, m), 1.27 (3H, s), 1.38 (3H, d, J=7.0 Hz), 1.53-1.70 (3H, m), 1.71-1.90 (3H, m), 2.08-2.58 (6H, m), 3.01 (1H, dd, J=13.9, 6.1 Hz), 3.21-3.38 (2H, m), 3.56 (1H, d, J=4.7 Hz), 3.82-3.94 (1H, m), 4.14-4.30 (2H, m), 4.69 (1H, brd, J=5.7 Hz), 5.16-5.29 (1H, m), 5.87 (1H, s), 7.11 (1H, d, J=10.0 Hz), 7.23-7.36 (3H, m), 7.38-7.46 (2H, m), 7.47-7.64 (5H, m);

MASS (ES+): m/e 619.55 (M+1).

### EXAMPLE 96

Compound E96 was obtained in a manner similar to Example 1.

¹H-NMR (300 MHz, CDCl₃, δ): 0.84 (3H, t, J=7.3 Hz), 1.09 (3×3H, s), 1.22 (3H, d, J=7 Hz), 1.29 (3H, s), 1.45 (2H, m), 1.58-1.91 (4H, m), 2.07-2.40 (6H, m), 2.90 (1H, dd, J=13.5, 6 Hz), 3.19 (1H, dd, J=13.5, 10 Hz), 3.26 (1H, m), 3.85 (2×3H, s), 3.86 (1H, m), 4.21 (1H, m), 4.27 (1H, q, J=7 Hz), 4.67 (1H, m), 5.16 (1H, ddd, J=10, 10, 6 Hz), 5.90 (1H, s), 6.62 (1H, d, J=15.5 Hz), 6.74-6.80 (3H, m), 6.86 (1H, dt, J=15.5, 7 Hz), 7.14 (1H, d, J=10 Hz), 7.30-7.48 (6H, m), 7.54 (1H, d, J=10 Hz), 7.57-7.68 (5H, m);

MASS (ES+): m/e 839.

### EXAMPLE 97

Compound E97 was obtained in a manner similar to Example 4.

¹H-NMR (300 MHz, CDCl₃, δ): 0.84 (3H, t, J=7.3 Hz), 1.10 (3×3H, s), 1.18 (3H, d, J=6.5 Hz), 1.18-1.32 (4H, m), 1.29 (3H, s), 1.45 (2H, m), 1.58-1.69 (1H, m), 1.70-1.89 (3H, m), 2.08-2.40 (4H, m), 2.51 (2H, m), 2.90 (1H, dd, J=13.5, 6 Hz), 3.20 (1H, dd, J=13.5, 10 Hz), 3.26 (1H, m), 3.85 (2×3H, s), 3.85 (1H, m), 4.14-4.25 (2H, m), 4.67 (1H, m), 5.15 (1H, ddd, J=10, 10, 6 Hz), 5.89 (1H, s), 6.75-6.82 (3H, m), 7.09 (1H, d, J=10 Hz), 7.32-7.50 (5H, m), 7.58 (1H, d, J=10 Hz), 7.58-7.69 (4H, s);

MASS (ES−): m/e 876 (M+Cl).

### EXAMPLE 98

Compound E98 was obtained in a manner similar to Example 6.

¹H-NMR (300 MHz, CDCl₃, δ): 0.85 (3H, t, J=7.5 Hz), 1.23-1.39 (4H, m), 1.29 (3×3H, s), 1.38 (3H, d, J=7 Hz), 1.54-1.71 (3H, m), 1.72-1.90 (3H, m), 2.08-2.24 (2H, m), 2.25-2.57 (4H, m), 2.89 (1H, dd, J=13.5, 6 Hz), 3.19 (1H, dd, J=13.5, 10 Hz), 3.26 (1H, m), 3.55 (1H, d, J=4.5 Hz), 3.85 (2×3H, s), 3.85 (1H, m), 4.14-4.29 (2H, m), 4.67 (1H, m), 5.15 (1H, ddd, J=10, 10, 6 Hz), 5.88 (1H, s), 6.74-6.79 (3H, m), 7.12 (1H, d, J=10 Hz), 7.55 (1H, d, J=10 Hz);

MASS (ES−): m/e 601;

[α]_{D}²⁴=−104.6° (c=0.32, CHCl₃).

### EXAMPLE 99

Compound E99 was obtained in a manner similar to Example 1.

¹H-NMR (300 MHz, CDCl₃, δ): 0.87 (3H, d, J=7 Hz), 0.88 (3H, t, J=7 Hz), 0.91 (3H, t, J=7 Hz), 1.09 (3×3H, s), 1.12-1.24 (2H, m), 1.22 (3H, d, J=6.5 Hz), 1.30 (3H, s), 1.38-1.52 (2H, m), 1.54-1.71 (1H, m), 1.74-2.10 (4H, m), 2.14-2.43 (6H, m), 3.53 (1H, m), 3.90 (1H, m), 4.21 (1H, m), 4.27 (1H, q, J=6.5 Hz), 4.59 (1H, dd, J=10.5, 10.5 Hz), 4.77 (1H, m), 5.87 (1H, s), 6.61 (1H, d, J=15, 5 Hz), 6.86 (1H, dt, J=15.5, 7 Hz), 7.19 (1H, d, J=10 Hz), 7.30-7.49 (7H, m), 7.56-7.69 (4H, m);

MASS (ES−) m/e 743.

### EXAMPLE 100

Compound E100 was obtained in a manner similar to Example 4.

¹H-NMR (300 MHz, CDCl₃, δ): 0.86 (3H, d, J=7 Hz), 0.88 (3H, t, J=7 Hz), 0.91 (3H, t, J=7 Hz), 1.10 (3×3H, s), 1.16-1.28 (3H, m), 1.18 (3H, d, J=6.5 Hz), 1.30 (3H, s), 1.37-1.70 (4H, m), 1.72-2.10 (4H, m), 2.11-2.43 (4H, m), 2.50 (2H, m), 3.53 (1H, dt, J=10, 7.5 Hz), 3.88 (1H, ddd, J=10, 10, 5 Hz), 4.18 (1H, m), 4.18 (1H, q, J=6.5 Hz), 4.58 (1H, dd, J=10.5, 10.5 Hz), 4.75 (1H, m), 5.88 (1H, s), 7.13 (1H, d, J=10 Hz), 7.32-7.48 (7H, m), 7.57-7.70 (4H, m);

MASS (ES−) m/e 745.

### EXAMPLE 101

Compound E101 was obtained in a manner similar to Example 6.

¹H-NMR (300 MHz, CDCl₃, δ): 0.86 (3H, d, J=7 Hz), 0.88 (3H, t, J=7 Hz), 0.91 (3H, t, J=7 Hz), 1.06-1.40 (5H, m), 1.30 (3H, s), 1.38 (3H, d, J=6.5 Hz), 1.50-2.10 (8H, m), 2.12-2.58 (6H, m), 3.53 (1H, dt, J=10, 7.5 Hz), 3.56 (1H, d, J=4.5 Hz), 3.89 (1H, ddd, J=10, 10, 5 Hz), 4.14-4.29 (2H, m), 4.58 (1H, dd, J=10.5, 10.5 Hz), 4.76 (1H, dd, J=8, 1.5 Hz), 5.91 (1H, s), 7.17 (1H, d, J=10.5 Hz), 7.38 (1H, d, J=10.5 Hz);

MASS (ES−) m/e 507;

[α]_{D}²⁴=−133.3° (c=0.25, CHCl₃).

### EXAMPLE 102

Compound E102 was obtained in a manner similar to Example 1.

¹H-NMR (300 MHz, CDCl₃, δ): 0.84 (3H, d, J=6.6 Hz), 0.86 (3H, t, J=7.3 Hz), 1.09 (3H, s), 1.22 (3H, d, J=6.6 Hz), 1.32-2.02 (9H, m), 2.09-2.46 (4H, m), 2.78 (1H, dd, J=14.5, 8 Hz), 3.16 (1H, dd, J=14.5, 8 Hz), 3.51 (1H, m), 3.76 (3H, s), 4.03 (1H, m), 4.26 (1H, m), 4.27 (1H, q, J=6.6 Hz), 4.48 (1H, dd, J=10.5, 10.5 Hz), 4.69 (1H, m), 4.72 (1H, m), 6.28 (1H, d, J=10.5 Hz), 6.29 (1H, d, J=10 Hz), 6.58 (1H, d, J=15.5 Hz), 6.80 (2×1H, d, J=8.5 Hz), 6.83 (1H, dt, J=15, 5.7 Hz), 7.11 (2×1H, d, J=8.5 Hz), 7.22 (1H, d, J=10.5 Hz), 7.30-7.48 (6H, m), 7.55-7.69 (4H, m);

MASS (ES−) m/e 821.

### EXAMPLE 103

Compound E103 was obtained in a manner similar to Example 4.

¹H-NMR (300 MHz, CDCl₃, δ): 0.84 (3H, d, J=6.6 Hz), 0.86 (3H, d, J=7.3 Hz), 1.10 (3×3H, s), 1.13-2.02 (13H, m), 1.18 (3H, d, J=6.5 Hz), 2.25-2.52 (4H, m), 2.78 (1H, dd, J=14.2, 7.7 Hz), 3.15 (1H, dd, J=14.2, 7.7 Hz), 3.51 (1H, m), 3.76 (3H, s), 4.02 (1H, m), 4.18 (3H, q, J=6.5 Hz), 4.22 (1H, m), 4.48 (1H, dd, J=10.6, 10.5 Hz), 4.68 (1H, ddd, J=9.7, 7.7, 7.7 Hz), 4.72 (1H, m), 6.29 (1H, d, J=9.7 Hz), 6.30 (1H, d, J=10.5 Hz), 6.80 (2×1H, d, J=8.8 Hz), 7.11 (2×1H, d, J=8.8 Hz), 7.16 (1H, d, J=10.7 Hz), 7.31-7.48 (6H, m), 7.57-7.67 (4H, m);

MASS (ES−) m/e 823.

### EXAMPLE 104

Compound E104 was obtained in a manner similar to Example 6.

¹H-NMR (300 MHz, CDCl₃, δ): 0.84 (3H, d, J=6.6 Hz), 0.86 (3H, t, J=7.4 Hz), 1.09 (1H, m), 1.18-1.32 (4H, m), 1.37 (3H, d, J=6.8 Hz), 1.49-2.03 (8H, m), 2.26-2.55 (4H, m), 2.79 (1H, dd, J=14.5, 7.9 Hz), 3.15 (1H, dd, J=14.5, 7.7 Hz), 3.51 (1H, m), 3.57 (1H, d, J=4.5 Hz), 3.77 (3H, s), 4.02 (1H, m), 4.17-4.29 (2H, m), 4.48 (1H, dd, J=10.7, 10.6 Hz), 4.68 (1H, m), 4.73 (1H, m), 6.30 (2×1H, brd, J=10 Hz), 6.81 (2×1H, d, J=8.5 Hz), 7.12 (2×1H, d, J=8.5 Hz), 7.20 (1H, d, J=10.6 Hz);

MASS (ES−) m/e 585;

[α]_{D}³⁰=−61.5° (c=0.33, CHCl₃)

### EXAMPLE 105

Compound E105 was obtained in a manner similar to Example 1.

¹H-NMR (300 MHz, CDCl₃, δ): 1.09 (3×3H, s), 1.24 (3H, d, J=7 Hz), 1.43 (2H, m), 1.61-1.89 (4H, m), 2.10-2.40 (4H, m), 2.77 (1H, dd, J=14, 7 Hz), 2.87 (1H, dd, J=13.5, 5 Hz), 3.08 (1H, m), 3.16 (1H, dd, J=14, 8 Hz), 3.18 (1H, dd, J=13.5, 11 Hz), 3.77 (3H, s), 3.94 (1H, m), 4.27 (1H, m), 4.27 (1H, q, J=7 Hz), 4.61 (1H, dd, J=8, 2.5 Hz), 4.69 (1H, ddd, J=10, 8, 7 Hz), 5.16 (1H, ddd, J=11, 10, 5 Hz), 6.30 (1H, d, J=10 Hz), 6.59 (1H, brd, J=16 Hz), 6.81 (2×1H, d, J=8.5 Hz), 6.84 (1H, dt, J=16, 7 Hz), 7.12 (2×1H, d, J=8.5 Hz), 7.12-7.48 (14H, m), 7.56-7.69 (4H, m);

MASS (ES−) m/e 855.

### EXAMPLE 106

Compound E106 was obtained in a manner similar to Example 4.

¹H-NMR (300 MHz, CDCl₃, δ): 1.10 (3×3H, s), 1.14-1.30 (4H, m), 1.18 (3H, d, J=7 Hz), 1.36-1.82 (6H, m), 2.10-2.40 (2H, m), 2.49 (2H, m), 2.77 (1H, dd, J=14.5, 7 Hz), 2.87 (1H, dd, J=13, 5.5 Hz), 3.02-3.24 (3H, m), 3.77 (3H, s), 3.94 (1H, m), 4.18 (1H, q, J=7 Hz), 4.24 (1H, m), 4.61 (1H, m), 4.69 (1H, m), 5.06 (1H, ddd, J=10, 10, 5.5 Hz), 6.29 (1H, d, J=9.5 Hz), 6.46 (1H, d), 6.81 (2×1H, d, J=9 Hz), 7.09-7.30 (8H, m), 7.32-7.48 (6H, m), 7.58-7.68 (4H, m);

MASS (ES−) m/e 857.

### EXAMPLE 107

Compound E107 was obtained in a manner similar to Example 6.

¹H-NMR (300 MHz, CDCl₃, δ): 1.20-1.36 (4H, m), 1.38 (3H, d, J=7 Hz), 1.54-1.88 (6H, m), 2.12-2.56 (4H, m), 2.78 (1H, dd, J=14.5, 7 Hz), 2.87 (1H, dd, J=13.5, 5.5 Hz), 3.02-3.24 (3H, m), 3.56 (1H, d, J=5 Hz), 3.94 (1H, m), 4.17-4.30 (2H, m), 4.61 (1H, m), 4.68 (1H, m), 5.06 (1H, ddd, J=10, 10, 5.5 Hz), 6.32 (1H, d, J=10 Hz), 6.46 (1H, d, J=10 Hz), 6.82 (2×1H, d, J=8.5 Hz), 7.08-7.32 (8H, m);

MASS (ES−) m/e 619;

[α]_{D}³⁰=−60.9° (C=0.31, CHCl₃).

### EXAMPLE 108

Compound E108 was obtained in a manner similar to Example 1.

¹H-NMR (300 MHz, CDCl₃, δ): 0.91 (3H, t, J=7.5 Hz), 1.09 (3H, s), 1.22 (3H, d, J=7 Hz), 1.36 (3H, s), 1.46 (2H, m), 1.56-1.72 (1H, m), 1.78-2.04 (3H, m), 2.12-2.54 (6H, m), 3.74 (1H, m), 4.04 (1H, m), 4.21-4.32 (2H, m), 4.75 (1H, m), 5.98 (1H, s), 6.19 (1H, d, J=10 Hz), 6.61 (1H, brd, J=16 Hz), 6.86 (1H, dt, J=16, 7 Hz), 7.16 (1H, d, J=10 Hz), 7.24-7.49 (11H, m), 7.56-7.68 (4H, m), 8.08 (1H, d, J=10 Hz);

MASS (ES−) m/e 763.

### EXAMPLE 109

Compound E109 was obtained in a manner similar to Example 4.

¹H-NMR (300 MHz, CDCl₃, δ): 0.91 (3H, t, J=7.3 Hz), 1.10 (3×3H, s), 1.18 (3H, d, J=7 Hz), 1.18-1.30 (4H, m), 1.36 (3H, s), 1.38-2.06 (6H, m), 2.09-2.58 (6H, m), 3.74 (1H, m), 4.03 (1H, m), 4.18 (1H, q, J=7 Hz), 4.26 (1H, m), 4.75 (1H, dd, J=8, 2 Hz), 5.98 (1H, s), 6.18 (1H, d, J=10 Hz), 7.10 (1H, d, J=10.5 Hz), 7.28-7.49 (11H, m), 7.58-7.69 (4H, m), 8.12 (1H, d, J=10 Hz);

MASS (ES−) m/e 765.

### EXAMPLE 110

Compound E110 was obtained in a manner similar to Example 6.

¹H-NMR (300 MHz, CDCl₃, δ): 0.92 (3H, t, J=7.3 Hz), 1.24-1.40 (4H, m), 1.36 (3H, s), 1.38 (3H, d, J=7 Hz), 1.53-1.68 (2H, m), 1.73-2.57 (10H, m), 3.55 (1H, d, J=5 Hz), 3.74 (1H, m), 4.04 (1H, m), 4.17-4.30 (2H, m), 4.76 (1H, dd, J=8.2 Hz), 5.99 (1H, s), 6.19 (1H, d, J=10.3 Hz), 7.14 (1H, d, J=10.6 Hz), 7.25-7.42 (5H, m), 8.10 (1H, d, J=10.3 Hz);

MASS (ES−) m/e 527;

[α]_{D}³⁰=−174.4° (c=0.22, CHCl₃).

### EXAMPLE 111

Compound E111 was obtained in a manner similar to Example 1.

¹H-NMR (300 MHz, CDCl₃, δ): 0.87 (3H, t, J=7.5 Hz), 0.96 (2H, m), 1.09 (3×3H, s), 1.12-1.32 (2H, m), 1.22 (3H, d, J=7 Hz), 1.29 (3H, s), 1.36-1.51 (2H, m), 1.54-2.00 (13H, m), 2.10-2.44 (6H, m), 3.52 (1H, dt, J=10, 7 Hz), 3.96 (1H, m), 4.21 (1H, dt, J=10, 7.5 Hz), 4.26 (1H, q, J=7 Hz), 4.74 (1H, dt, J=8, 2 Hz), 5.00 (1H, d, J=10, 8 Hz), 5.85 (1H, s), 6.81 (1H, d, J=16 Hz), 6.86 (1H, dt, J=16, 7 Hz), 7.14 (1H, d, J=10 Hz), 7.30-7.50 (7H, m), 7.56-7.69 (4H, m);

MASS (ES−) m/e 783.

### EXAMPLE 112

Compound E112 was obtained in a manner similar to Example 4.

¹H-NMR (300 MHz, CDCl₃, δ): 0.86 (3H, t, J=7.3 Hz), 0.96 (2H, m), 1.10 (3×3H, s), 1.13-1.34 (6H, m), 1.18 (3H, d, J=6.5 Hz), 1.29 (3H, s), 1.45 (2H, m), 1.52-2.00 (13H, m), 2.08-2.43 (4H, m), 2.50 (2H, m), 3.52 (1H, dt, J=10.5, 7 Hz), 3.96 (1H, m), 4.18 (1H, m), 4.18 (1H, q, J=6.5 Hz), 4.74 (1H, dd, J=8, 2 Hz), 5.00 (1H, dd, J=10, 7.5 Hz), 5.85 (1H, s), 7.09 (1H, d, J=10 Hz), 7.31-7.48 (7H, m), 7.57-7.67 (4H, m);

MASS (ES−) m/e 785.

### EXAMPLE 113

Compound E113 was obtained in a manner similar to Example 6.

¹H-NMR (300 MHz, CDCl₃, δ): 0.87 (3H, t, J=7.3 Hz), 0.96 (2H, m), 1.08-1.40 (8H, m), 1.29 (3H, s), 1.38 (3H, d, J=7.2 Hz), 1.50-2.00 (13H, m), 2.08-2.57 (6H, m), 3.52 (1H, ddd, J=10, 7.5, 7 Hz), 3.56 (1H, d, J=5 Hz), 3.96 (1H, m), 4.13-4.28 (2H, m), 4.74 (1H, dd, J=8, 2 Hz), 4.99 (1H, dt, J=10, 8 Hz), 5.88 (1H, s), 7.12 (1H, d, J=10 Hz), 7.34 (1H, d, J=10 Hz);

MASS (ES−) m/e 547.

### EXAMPLE 114

Compound E114 was obtained in a manner similar to Example 1.

¹H-NMR (300 MHz, CDCl₃, δ): 0.80 (3H, t, J=7.3 Hz), 0.86 (3H, t, J=7.3 Hz), 0.96 (2H, m), 1.10 (3×3H, m), 1.17 (2H, m), 1.42 (2H, m), 1.52-2.00 (15H, m), 2.10-2.44 (6H, m), 3.52 (1H, dt, J=10, 7 Hz), 3.96 (1H, m), 4.15 (1H, t, J=6 Hz), 4.20 (1H, dt, J=10.5, 7.5 Hz), 4.74 (1H, dd, J=8, 2 Hz), 5.00 (1H, dt, J=10, 8 Hz), 5.85 (1H, s), 6.54 (1H, brd, J=16 Hz), 6.79 (1H, dt, J=16, 7 Hz), 7.14 (1H, d, J=10.5 Hz), 7.28-7.48 (7H, m), 7.54-7.68 (4H, m);

MASS (ES−) m/e 797.

### EXAMPLE 115

Compound E115 was obtained in a manner similar to Example 4.

¹H-NMR (300 MHz, CDCl₃, δ): 0.81 (3H, t, J=7.5 Hz), 0.86 (3H, t, J=7.3 Hz), 0.96 (2H, m), 1.11 (3×3H, s), 1.12-1.27 (6H, m), 1.29 (3H, s), 1.37 (2H, m), 1.47-1.98 (15H, m), 2.07-2.49 (6H, m), 3.52 (1H, dt, J=10, 7 Hz), 3.95 (1H, m), 4.10 (1H, t, J=7 Hz), 4.16 (1H, dt, J=10, 7 Hz), 4.73 (1H, dd, J=8, 2 Hz), 4.99 (1H, dt, J=10, 7 Hz), 5.84 (1H, s), 7.08 (1H, d, J=10 Hz), 7.32-7.48 (7H, m), 7.58-7.66 (4H, m);

MASS (ES+): m/e 799.

### EXAMPLE 116

Compound E116 was obtained in a manner similar to Example 6.

¹H-NMR (300 MHz, CDCl₃, δ): 0.87 (3H, t, J=7.3 Hz), 0.94 (3H, t, J=7 Hz), 0.94 (2H, m), 1.08-1.40 (8H, m), 1.29 (3H, s), 1.50-2.00 (15H, m), 2.07-2.50 (6H, m), 3.49 (1H, d, J=4.5 Hz), 3.52 (1H, m), 3.96 (1H, m), 4.10-4.25 (2H, m), 4.74 (1H, dd, J=7.5, 2 Hz), 4.99 (1H, dt, J=10, 7.5 Hz), 5.88 (1H, s), 7.12 (1H, d, J=10 Hz), 7.34 (1H, d, J=10 Hz);

MASS (ES−) m/e 561.

### EXAMPLE 117

Compound E117 was obtained in a manner similar to Example 1.

¹H-NMR (300 MHz, CDCl₃, δ): 1.09 (3×3H, s), 1.22 (3H, d, J=7 Hz), 1.24-1.92 (14H, m), 1.96-2.39 (5H, m), 2.62 (1H, m), 2.95 (1H, dd, J=13.5, 6 Hz), 3.21 (1H, m), 3.25 (1H, dd, J=13.5, 10 Hz), 3.93 (1H, m), 4.22 (1H, m), 4.27 (1H, q, J=7 Hz), 4.66 (1H, m), 5.16 (1H, ddd, J=10, 10, 6 Hz), 5.74 (1H, s), 6.62 (1H, d, J=16 Hz), 6.87 (1H, dt, J=16, 7 Hz), 7.15-7.29 (6H, m), 7.29-7.48 (7H, m), 7.56-7.68 (4H, m);

MASS (ES−) m/e 804.

### EXAMPLE 118

Compound E118 was obtained in a manner similar to Example 4.

¹H-NMR (300 MHz, CDCl₃, δ): 1.10 (3×3H, s), 1.18 (3H, d, J=7 Hz), 1.20-1.68 (14H, m), 1.69-1.92 (4H, m), 2.04 (1H, m), 2.18 (1H, m), 2.32 (1H, m), 2.51 (2H, m), 2.63 (1H, m), 2.95 (1H, dd, J=14, 6 Hz), 3.21 (1H, m), 3.25 (1H, dd, J=14, 10 Hz), 3.92 (1H, m), 4.18 (1H, q, J=7 Hz), 4.20 (1H, m), 4.66 (1H, m), 5.16 (1H, ddd, J=10, 10, 6 Hz), 5.73 (1H, s), 7.13 (1H, s), 7.17-7.31 (5H, m), 7.33-7.48 (7H, m), 7.59-7.68 (4H, m);

MASS (ES−) m/e 805.

### EXAMPLE 119

Compound E119 was obtained in a manner similar to Example 6.

¹H-NMR (300 MHz, CDCl₃, δ): 1.20-1.92 (19H, m), 1.94-2.70 (5H, m), 2.95 (1H, dd, J=13.5, 6 Hz), 3.20 (1H, m), 3.24 (1H, dd, J=13.5, 10 Hz), 3.56 (1H, d, J=4.5 Hz), 3.92 (1H, m), 4.15-4.29 (2H, m), 4.64 (1H, m), 5.16 (1H, ddd, J=10, 10, 6 Hz), 5.75 (1H, s), 7.17 (1H, d, J=10 Hz), 7.19-7.32 (5H, m), 7.38 (1H, d, J=10 Hz);

MASS (ES−) m/e 567;

[α]_{D}²⁵=−98.8° (c=0.33, CHCl₃).

### EXAMPLE 120

Compound E120 was obtained in a manner similar to Example 1.

¹H-NMR (300 MHz, CDCl₃, δ): 0.81 (3H, t, J=7.5 Hz), 1.11 (3×3H, s), 1.23-1.93 (16H, m), 1.96-2.37 (5H, m), 2.64 (1H, m), 2.96 (1H, dd, J=13, 6 Hz), 3.15-3.31 (2H, m), 3.93 (1H, m), 4.16 (1H, t, J=6 Hz), 4.22 (1H, m), 4.66 (1H, m), 5.17 (1H, m), 5.72 (1H, s), 6.56 (1H, d, J=16 Hz), 6.81 (1H, dt, J=16, 7 Hz), 7.15-7.48 (13H, m), 7.55-7.69 (4H, m);

MASS (ES+): m/e 819.

### EXAMPLE 121

Compound E121 was obtained in a manner similar to Example 4.

¹H-NMR (300 MHz, CDCl₃, δ): 0.81 (3H, t, J=7.3 Hz), 1.11 (3×3H, s), 1.14-1.90 (20H, m), 1.95-2.23 (2H, m), 2.26-2.49 (3H, m), 2.64 (1H, m), 2.95 (1H, dd, J=13.5, 6 Hz), 3.21 (1H, m), 3.25 (1H, dd, J=13.5, 10 Hz), 3.91 (1H, m), 4.11 (1H, t, J=6 Hz), 4.18 (1H, m), 4.66 (1H, m), 5.16 (1H, ddd, J=10, 10, 6 Hz), 5.69 (1H, s), 7.12 (1H, d, J=10 Hz), 7.16-7.31 (5H, m), 7.32-7.48 (7H, m), 7.57-7.67 (4H, m);

MASS (ES+): m/e 819.

### EXAMPLE 122

Compound E122 was obtained in a manner similar to Example 6.

¹H-NMR (300 MHz, CDCl₃, δ): 0.94 (3H, t, J=7.4 Hz), 1.20-1.95 (20H, m), 2.03 (1H, m), 2.16 (1H, m), 2.31 (1H, m), 2.44 (2H, m), 2.62 (1H, m), 2.95 (1H, dd, J=14, 6 Hz), 3.14-3.30 (2H, m), 3.49 (1H, d, J=5 Hz), 3.92 (1H, m), 4.08-4.26 (2H, m), 4.68 (1H, m), 5.16 (1H, ddd, J=10, 10, 6 Hz), 5.72 (1H, s), 7.12-7.31 (5H, m), 7.16 (1H, d, J=10 Hz), 7.38 (1H, d, J=10 Hz);

MASS (ES−) m/e 581;

[α]_{D}²⁵=−100.4° (c=0.30, CHCl₃).

### EXAMPLE 123

Compound E123 was obtained in a manner similar to Example 1.

¹H-NMR (300 MHz, CDCl₃, δ): 0.90 (2H, m), 1.06-1.32 (4H, m), 1.10 (9H, s), 1.23 (3H, d, J=7 Hz), 1.36-1.52 (3H, m), 1.56-1.82 (10H, m), 2.14-2.39 (4H, m), 2.94 (1H, dd, J=14, 5 Hz), 3.10 (1H, m), 3.23 (1H, dd, J=14, 10 Hz), 3.94 (1H, m), 4.27 (1H, m), 4.27 (1H, q, J=7 Hz), 4.52 (1H, m), 4.62 (1H, m), 5.09 (1H, ddd, J=10, 10, 5 Hz), 6.04 (1H, d, J=10 Hz), 6.48 (1H, d, J=10 Hz), 6.61 (1H, d, J=16 Hz), 6.87 (1H, dt, J=16, 7 Hz), 7.16-7.50 (1.2H, m), 7.57-7.70 (4H, m);

MASS (ES+): m/e 855.

### EXAMPLE 124

Compound E124 was obtained in a manner similar to Example 4.

¹H-NMR (300 MHz, CDCl₃, δ): 0.92 (2H, m), 1.08-1.32 (8H, m), 1.10 (9H, s), 1.19 (3H, d, J=7 Hz), 1.38-1.50 (3H, m), 1.58-1.84 (10H, m), 2.19 (2H, m), 2.32 (2H, m), 2.51 (2H, brt, J=7 Hz), 2.94 (1H, dd, J=13, 5 Hz), 3.10 (1H, m), 3.23 (1H, dd, J=13, 10 Hz), 3.93 (1H, m), 4.18 (1H, q, J=7 Hz), 4.25 (1H, dt, J=10, 7 Hz), 4.52 (1H, dt, J=11, 8 Hz), 4.62 (1H, m), 5.09 (1H, ddd, J=10, 10, 5 Hz), 6.06 (1H, d, J=10 Hz), 6.51 (1H, d, J=11 Hz), 7.13 (1H, d, J=10 Hz), 7.18-7.32 (5H, m), 7.34-7.48 (6H, m), 7.59-7.67 (4H, m);

MASS (ES−) m/e 833.

### EXAMPLE 125

Compound E125 was obtained in a manner similar to Example 6.

¹H-NMR (300 MHz, CDCl₃, δ): 0.92 (1H, m), 1.07-1.50 (10H, m), 1.38 (3H, d, J=7 Hz), 1.54-1.90 (11H, m), 2.18 (1H, m), 2.33 (1H, m), 2.46 (2H, m), 2.93 (1H, dd, J=13, 5 Hz), 3.10 (1H, m), 3.22 (1H, dd, J=13, 10 Hz), 3.56 (1H, d, J=5 Hz), 3.93 (1H, m), 4.18-4.31 (2H, m), 4.52 (1H, dt, J=10, 7 Hz), 4.62 (1H, m), 5.08 (1H, ddd, J=10, 10, 5 Hz), 6.08 (1H, d, J=10 Hz), 6.49 (1H, d, J=10 Hz), 7.16 (1H, d, J=10 Hz), 7.17-7.32 (5H, m);

MASS (ES−) m/e 595;

[α]_{D}²³=−53.8° (c=0.09, CHCl₃).

### EXAMPLE 126

Compound E126 was obtained in a manner similar to Example 1.

¹H-NMR (300 MHz, CDCl₃, δ): 0.80 (3H, t, J=7 Hz), 0.90 (2H, m), 1.04-1.32 (4H, m), 1.10 (9H, s), 1.36-1.50 (3H, m), 1.52-1.90 (12H, m), 2.10-2.36 (4H, m), 2.96 (1H, dd, J=13, 5 Hz), 3.10 (1H, m), 3.22 (1H, dd, J=13, 10 Hz), 3.93 (1H, m), 4.15 (1H, t, J=6 Hz), 4.27 (1H, ddd, J=10, 8, 7 Hz), 4.52 (1H, ddd, J=10, 8, 7 Hz), 4.62 (1H, m), 5.09 (1H, ddd, J=10, 10, 5 Hz), 6.05 (1H, d, J=10 Hz), 6.48 (1H, d, J=10 Hz), 6.53 (1H, d, J=16 Hz), 6.79 (1H, dt, J=16, 7 Hz), 7.14-7.47 (12H, m), 7.54-7.68 (4H, m);

MASS (ES−) m/e 845.

### EXAMPLE 127

Compound E127 was obtained in a manner similar to Example 4.

¹H-NMR (300 MHz, CDCl₃, δ): 0.81 (3H, t, J=7 Hz), 0.90 (2H, m), 1.11 (9H, s), 1.12-1.82 (23H, m), 2.15-2.47 (4H, m), 2.94 (1H, dd, J=13, 5 Hz), 3.10 (1H, m), 3.22 (1H, dd, J=13, 10 Hz), 3.93 (1H, m), 4.11 (1H, t, J=6 Hz), 4.24 (1H, dt, J=10, 7 Hz), 4.52 (1H, dt, J=10, 7 Hz), 4.62 (1H, m), 5.09 (1H, ddd, J=10, 10, 5 Hz), 6.06 (1H, d, J=10 Hz), 6.51 (1H, d, J=10 Hz), 7.12 (1H, d, J=10 Hz), 7.18-7.32 (5H, m), 7.33-7.47 (6H, m), 7.58-7.66 (4H, m);

MASS (ES−) m/e 847.

### EXAMPLE 128

Compound E128 was obtained in a manner similar to Example 6.

¹H-NMR (300 MHz, CDCl₃, δ): 0.80-1.00 (2H, m), 0.94 (3H, t, J=7 Hz), 1.06-1.96 (23H, m), 2.18 (1H, m), 2.31 (1H, m), 2.44 (2H, m), 2.93 (1H, dd, J=13, 5 Hz), 3.09 (1H, m), 3.22 (1H, dd, J=13, 10 Hz), 3.51 (1H, d, J=5 Hz), 3.93 (1H, m), 4.15 (1H, m), 4.26 (1H, dt, J=10, 8 Hz), 4.52 (1H, dt, J=10, 7 Hz), 4.62 (1H, m), 5.08 (1H, ddd, J=10, 10, 5 Hz), 6.08 (1H, d, J=10 Hz), 6.50 (1H, d, J=10 Hz), 7.16 (1H, d, J=10 Hz), 7.16-7.33 (5H, m);

MASS (ES−) m/e 609;

[α]_{D}²³=−49.6° (c=0.26, CHCl₃).

### EXAMPLE 129

Compound E129 was obtained in a manner similar to Example 1.

¹H-NMR (300 MHz, CDCl₃, δ): 0.90 (2H, m), 1.09 (9H, s), 1.11-1.33 (8H, m), 1.22 (3H, d, J=7 Hz), 1.36-1.52 (3H, m), 1.59-1.90 (6H, m), 2.14-2.38 (4H, m), 2.94 (1H, dd, J=13, 5 Hz), 3.10 (1H, m), 3.22 (1H, dd, J=13, 6 Hz), 3.94 (1H, m), 4.22-4.33 (2H, m), 4.52 (1H, dt, J=8, 7 Hz), 4.62 (1H, m), 5.09 (1H, ddd, J=10, 6, 5 Hz), 6.04 (1H, d, J=10 Hz), 6.48 (1H, d, J=10 Hz), 6.60 (1H, d, J=16 Hz), 6.86 (1H, dt, J=16, 7 Hz), 7.15-7.48 (12H, m);

MASS (ES+): m/e 833.

### EXAMPLE 130

Compound E130 was obtained in a manner similar to Example 4.

¹H-NMR (300 MHz, CDCl₃, δ): 0.89 (2H, m), 1.05-1.34 (8H, m), 1.10 (9H, s), 1.18 (3H, d, J=7 Hz), 1.37-1.52 (3H, m), 1.58-1.85 (10H, m), 2.12-2.38 (2H, m), 2.50 (2H, m), 2.93 (1H, dd, J=13, 5 Hz), 3.10 (1H, m), 3.22 (1H, dd, J=13, 11 Hz), 3.93 (1H, m), 4.18 (1H, q, J=7 Hz), 4.24 (1H, dt, J=10, 8 Hz), 4.52 (1H, dt, J=10, 7 Hz), 4.62 (1H, m), 5.08 (1H, ddd, J=11, 10, 5 Hz), 6.06 (1H, d, J=10 Hz), 6.50 (1H, d, J=10 Hz), 7.12 (1H, d, J=10 Hz), 7.17-7.32 (5H, m), 7.33-7.48 (6H, m), 7.58-7.67 (4H, m);

MASS (ES−) m/e 833.

### EXAMPLE 131

Compound E131 was obtained in a manner similar to Example 6.

¹H-NMR (300 MHz, CDCl₃, δ): 0.90 (2H, m), 1.06-1.90 (21H, m), 1.38 (3H, d, J=7 Hz), 2.18 (1H, m), 2.27-2.58 (3H, m), 2.93 (1H, dd, J=13, 5 Hz), 3.10 (1H, m), 3.22 (1H, dd, J=13, 10 Hz), 3.58 (1H, brd, J=3 Hz), 3.93 (1H, m), 4.18-4.32 (2H, m), 4.52 (1H, dt, J=10, 8 Hz), 4.62 (1H, m), 5.08 (1H, ddd, J=10, 10, 5 Hz), 6.12 (1H, d, J=10 Hz), 6.51 (1H, d, J=10 Hz), 7.13-7.33 (6H, m);

MASS (ES−) m/e 595;

[α]_{D}²³=−46.4° (c=1.39, CHCl₃).

### EXAMPLE 132

Compound E132 was obtained in a manner similar to Example 1.

¹H-NMR (300 MHz, CDCl₃, δ): 0.80 (3H, t, J=7.5 Hz), 0.87 (3H, t, J=7.5 Hz), 1.10 (9H, s), 1.29 (3H, s), 1.42 (2H, m), 1.54-1.69 (3H, m), 1.74-1.92 (3H, m), 1.98-2.42 (8H, m), 2.65 (1H, m), 3.32 (1H, m), 3.75 (1H, m), 4.15 (1H, t, J=6 Hz), 4.21 (1H, m), 4.72 (1H, m), 4.85 (1H, m), 5.83 (1H, s), 6.54 (1H, d, J=16 Hz), 6.80 (1H, dt, J=16, 7 Hz), 7.11 (1H, d, J=10 Hz), 7.15-7.23 (3H, m), 7.25-7.47 (9H, m), 7.55-7.67 (4H, m);

MASS (ES+): m/e 829.

### EXAMPLE 133

Compound E133 was obtained in a manner similar to Example 4.

¹H-NMR (300 MHz, CDCl₃, δ): 0.81 (3H, t, J=7.5 Hz), 0.87 (3H, t, J=7.3 Hz), 1.11 (9H, s), 1.14-1.25 (4H, m), 1.28 (3H, s), 1.37 (2H, m), 1.48-1.92 (6H, m), 2.00-2.25 (4H, m), 2.26-2.49 (4H, m), 2.64 (2H, m), 3.32 (1H, m), 3.76 (1H, m), 4.10 (1H, t, J=6 Hz), 4.17 (1H, dt, J=10, 7 Hz), 4.72 (1H, m), 4.84 (1H, dt, J=10, 7 Hz), 5.82 (1H, s), 7.05 (1H, d, J=10 Hz), 7.14-7.22 (3H, m), 7.24-7.48 (9H, m), 7.57-7.66 (4H, m);

MASS (ES−) m/e 807.

### EXAMPLE 134

Compound E134 was obtained in a manner similar to Example 6.

¹H-NMR (300 MHz, CDCl₃, δ): 0.88 (3H, t, J=7 Hz), 0.94 (3H, t, J=7 Hz), 1.22-1.40 (4H, m), 1.29 (3H, s), 1.52-1.70 (4H, m), 1.74-1.98 (4H, m), 2.01-2.26 (4H, m), 2.29-2.50 (4H, m), 2.65 (2H, m), 3.33 (1H, m), 3.50 (1H, d, J=5 Hz), 3.75 (1H, m), 4.08-4.26 (2H, m), 4.73 (1H, m), 4.85 (1H, ddd, J=10, 8, 7 Hz), 5.84 (1H, s), 7.09 (1H, d, J=10 Hz), 7.15-7.24 (3H, m), 7.25-7.33 (2H, m), 7.42 (1H, d, J=10 Hz);

MASS (ES−) m/e 569.

### EXAMPLE 135

Compound E135 was obtained in a manner similar to Example 1.

¹H-NMR (300 MHz, CDCl₃, δ): 0.87 (3H, t, J=7.5 Hz), 1.09 (9H, s), 1.21 (3H, d, J=7 Hz), 1.29 (3H, s), 1.45 (2H, m), 1.64 (1H, m), 1.75-1.92 (3H, m), 2.00-2.42 (8H, m), 2.65 (2H, m), 3.32 (1H, m), 3.75 (1H, m), 4.21 (1H, m), 4.27 (1H, q, J=7 Hz), 4.72 (1H, m), 4.85 (1H, dt, J=10, 7.5 Hz), 5.83 (1H, s), 6.61 (1H, d, J=16 Hz), 6.86 (1H, dt, J=16, 7 Hz), 7.11 (1H, d, J=10 Hz), 7.15-7.23 (3H, m), 7.24-7.49 (9H, m), 7.56-7.69 (4H, m);

MASS (ES+) m/e 815.

### EXAMPLE 136

Compound E136 was obtained in a manner similar to Example 4.

¹H-NMR (300 MHz, CDCl₃, δ): 0.87 (3H, t, J=7 Hz), 1.10 (9H, s), 1.18 (3H, d, J=7 Hz), 1.20-1.32 (4H, m), 1.28 (3H, s), 1.38-1.52 (3H, m), 1.72-1.91 (3H, m), 2.00-2.42 (6H, m), 2.50 (2H, m), 2.64 (2H, m), 3.34 (1H, m), 3.74 (1H, m), 4.18 (1H, q, J=7 Hz), 4.18 (1H, m), 4.72 (1H, m), 4.84 (1H, m), 5.83 (1H, s), 7.05 (1H, d, J=10 Hz), 7.14-7.22 (3H, m), 7.24-7.32 (2H, m), 7.33-7.49 (7H, m), 7.58-7.67 (4H, m);

MASS (ES−) m/e 793.

### EXAMPLE 137

Compound E137 was obtained in a manner similar to Example 6.

¹H-NMR (300 MHz, CDCl₃, δ): 0.87 (3H, t, J=7.5 Hz), 1.20-1.40 (4H, m), 1.29 (3H, s), 1.38 (3H, d, J=7 Hz), 1.53-1.69 (3H, m), 1.75-1.92 (3H, m), 1.98-2.25 (4H, m), 2.26-2.55 (4H, m), 2.64 (2H, m), 3.32 (1H, m), 3.56 (1H, d, J=4.5 Hz), 3.74 (1H, m), 4.10-4.29 (2H, m), 4.72 (1H, m), 4.84 (1H, ddd, J=10, 7.5, 7.5 Hz), 5.84 (1H, s), 7.08 (1H, d, J=10 Hz), 7.12-7.23 (3H, m), 7.24-7.33 (2H, m), 7.42 (1H, d, J=10 Hz);

MASS (ES−) m/e 555.

### EXAMPLE 138

Compound E138 was obtained in a manner similar to the method disclosed in WO00/21979.

### EXAMPLE 139

To a stirred solution of Compound E138 (506 mg) in methanol (10 ml) was added aqueous sodium periodate (1.08 M, 2 ml) at ambient temperature and the resulting mixture was stirred at the same temperature overnight. The solvent was removed under reduced pressure and the residue was dissolved in ethyl acetate and 1N hydrochloric acid. The organic layer was separated, washed with brine, dried over sodium sulfate, filtered and evaporated under reduced pressure. The crude product was purified by flash chromatography eluting with 5% methanol/chloroform (v/v) as a solvent mixture to give the Compound E139 (480 mg) as a white amorphous solid.

¹H-NMR (300 MHz, CDCl₃, δ): 0.84 (3H, t, J=7.5 Hz), 0.88 (3H, d, J=6.5 Hz), 1.20-1.42 (5H, m), 1.28 (3H, s), 1.53-1.70 (3H, m), 1, 81 (1H, m), 2.17 (1H, m), 2.24-2.42 (4H, m), 2.62 (1H, m), 2.73 (1H, dd, J=9.5 and 8 Hz), 2.96 (1H, dd, J=13.5, 6 Hz), 3.23 (1H, dd, J=13.5, 10 Hz), 4.05 (1H, dd, J=9.5, 7.5 Hz), 4.23 (1H, m), 4.69 (1H, dd, J=8.2 Hz), 5.16 (1H, ddd, J=10, 10, 6 Hz), 6.10 (1H, s), 7.16-7.32 (6H, m), 7.6 0(1H, d, J=10 Hz);

MASS (ES+): m/e 529.

### EXAMPLE 140

To a stirred solution of Compound E138 (1000 mg) in methanol (20 ml) was added aqueous solution of sodium periodate (1.08 M, 2 ml) at ambient temperature and the resulting mixture was stirred at the same temperature overnight. The solution was concentrated in vacuo and the residue was dissolved in ethyl acetate and added 1 N hydrochloric acid. The organic layer was separated, washed with brine, dried over sodium sulfate, filtered and evaporated under reduced pressure. The crude product was purified by flash chromatography using 5% methanol/chloroform (v/v) as a solvent mixture to give the Compound E140 (949 mg) as a white amorphous solid.

¹H-NMR (300 MHz, CDCl₃, δ): 0.84 (3H, t, J=7.5 Hz), 0.87 (3H, d, J=7 Hz), 1.21-1.45 (5H, m), 1.28 (3H, s), 1.53-1.70 (3H, m), 1.82 (1H, m), 2.17 (1H, m), 2.25-2.42 (4H, m), 2.62 (1H, m), 2.72 (1H, dd, J=9, 8 Hz), 2.96 (1H, dd, J=13, 6 Hz), 3.23 (1H, dd, J=13, 10 Hz), 4.05 (1H, dd, J=9, 7 Hz), 4.22 (1H, m), 4.68 (1H, dd, J=7, 2 Hz), 5.15 (1H, ddd, J=10, 9, 6 Hz), 6.04 (1H, s), 7.15-7.32 (6H, m), 7.58 (1H, d, J=9 Hz);

MASS (ES+): m/e 529.

### EXAMPLE 141

To a stirred solution of Compound E140 in dichloromethane (2 ml) was aded N-methylhydroxylamine hydrochloride (18 mg), 1-hydroxybenzotriazole (58.2 mg) and a solution of 1-ethyl-3-(3′-dimethylaminopropyl)carbodiimide (67.0 mg) in dichloromethane at ambient temperature and the resulting mixture was stirred at the same temperature for three days. This mixture was poured into water and the organic layer was separated. The organic layer was washed with brine, dried over sodium sulfate, filtered and evaporated under reduced pressure. The residue was purified by flash chromatography using 9% methanol/chloroform (v/v) as a solvent mixture to give the Compound E141 (18 mg) as a white amorphous solid.

¹H-NMR (300 MHz, CDCl₃, δ): 0.84 (3H, t, J=7.5 Hz), 0.88 (3H, d, J=6.5 Hz), 1.20-1.44 (5H, m), 1.28 (3H, s), 1.52-1.90 (5H, m), 2.15 (1H, m), 2.25-2.42 (3H, m), 2.62 (1H, m), 2.73 (1H, dd, J=9.5, 7.5 Hz), 2.82 (1H, s), 2.96 (1H, dd, J=13.5, 6 Hz), 3.24 (1H, dd, J=13.5, 10 Hz), 4.06 (1H, dd, J=9.5, 8 Hz), 4.19 (1H, dt, J=10, 7.5 Hz), 4.67 (1H, dd, J=8, 2 Hz), 5.16 (1H, ddd, J=10, 10, 6 Hz), 5.83 (1H, s), 7.16 (1H, d, J=10 Hz), 7.19-7.33 (5H, m), 7.54 (1H, d, J=10 Hz);

MASS (ES−) m/e 556;

[α]_{D}²¹=−130.8° (c=0.30, CHCl₃).

### EXAMPLE 142

To a stirred solution of Compound E139 (473 mg) in dichloromethane (5 ml) was added 1-hydroxybenzotriazole (181 mg), a solution of 1-ethyl-3-(3′-dimethylaminopropyl)carbodiimide (208 mg) in chloroform and N,O-dimethylhydroxylamine hydrochloric acid (131 mg) at ambient temperature and the resulting mixture was left at the same temperature for two weeks. This mixture was poured into 10% aqueous solution of citric acid and the organic layer was separated, washed with saturated aqueous sodium bicarbonate solution and brine. The organic layer was dried over sodium sulfate, filtered and evaporated under reduced pressure. The residue was purified by flash chromatography eluting with ethyl acetate as an eluting solvent to give the Compound E142 (453 mg) as a white amorphous solid.

¹H-NMR (300 MHz, CDCl₃, δ): 0.84 (3H, t, J=7.5 Hz), 0.88 (3H, d, J=6.6. Hz), 1.25-1.44 (6H, m), 1.29 (3H, s), 1.55-1.69 (2H, m), 1.83 (1H, m), 2.14 (1H, m), 2.26-2.45 (4H, m), 2.65 (1H, m), 2.73 (1H, dd, J=9.5, 8 Hz), 2.96 (1H, dd, J=13.5, 6 Hz), 3.18 (3H, s), 3.24 (1H, dd, J=13.5, 10 Hz), 3.68 (3H, s), 4.06 (1H, dd, J=9.5, 7.3 Hz), 4.21 (1H, m), 4.67 (1H, dd, J=8, 2 Hz), 5.16 (1H, ddd, J=10.3, 10, 6 Hz), 5.81 (1H, s), 7.14 (1H, d, J=10.2 Hz), 7.16-7.32 (5H, m), 7.56 (1H, d, J=10.3 Hz);

MASS (ES+): m/e 572.

### EXAMPLE 143

To a stirred solution of the Compound E142 (97 mg) in tetrahydrofuran (4 ml) was added ethyl magnesium bromide (1.04 M in tetrahydrofuran, 1.6 ml) dropwise at −78° C. and the mixture was allowed to warm to 0° C. The reaction mixture was quenched with saturated aqueous sodium bicarbonate solution at ambient temperature and the resulting mixture was extracted with ethyl acetate. The organic layer was washed with brine, dried over sodium sulfate, filtered and evaporated under reduced pressure. The residue was purified by preparative thin layer chromatography using 90% ethyl acetate/hexane (v/v) as a solvent mixture to give the Compound E143 (38 mg) as a white amorphous solid.

¹H-NM (300 MHz, CDCl₃, δ): 0.84 (3H, 7, J=7.3 Hz), 0.88 (3H, d, J=6.6 Hz), 1.05 (3H, t, J=7.3 Hz), 1.20-1.44 (6H, m), 1.28 (3H, s), 1.48-1.71 (3H, m), 1.81 (1H, m), 2.14 (1H, m), 2.26-2.46 (5H, m), 2.63 (1H, m), 2.73 (1H, dd, J=9.5, 8 Hz), 2.96 (1H, dd, J=13.5, 6 Hz), 3.24 (1H, dd, J=13.5, 10 Hz), 4.06 (1H, dd, J=9.5, 7.5 Hz), 4.19 (1H, dt, J=10, 7.5 Hz), 4.67 (1H, dd, J=8, 2 Hz), 5.16 (1H, ddd, J=10, 10.6 Hz), 5.79 (1H, s), 7.14 (1H, d, J=10 Hz), 7.18-7.32 (5H, m), 7.54 (1H, d, J=10 Hz);

MASS (ES+): m/e 541.

### EXAMPLE 144

To a stirred solution of dimethyl 3-fluoro-2-oxopropylphosphonate (86.1 mg) in 2-propanol (3 ml) was added cesium carbonate (152 mg) at ambient temperature and the mixture was stirred at the same temperature for half an hour. To the resulting light yellow solution was added a solution of the starting compound (Compound (105)) (200 mg) in isopropyl alcohol at the same temperature and the mixture was stirred at the same temperature for two hours. The reaction mixture was quenched with 10% aqueous solution of citric acid, diluted with ethyl acetate and water. The organic layer was separated and washed with saturated sodium bicarbonate solution and brine. The organic layer was dried over sodium sulfate, filtered and evaporated under reduced pressure. The residue was purified by preparative thin layer chromatography using 66% ethyl acetate/hexane (v/v) as a solvent mixture to give Compound E144 (68 mg) as a white amorphous solid.

¹H-NMR (300 MHz, CDCl₃, δ): 0.83 (3H, t, J=7 Hz), 1.29 (3H, s), 1.50 (2H, m), 1.64-1.92 (4H, m), 2.08-2.40 (6H, m), 2.97 (1H, dd, J=13.5, 6 Hz), 3.24 (1H, dd, J=13.5, 9.5 Hz), 3.26 (1H, m), 3.85 (1H, m), 4.24 (1H, ddd, J=10, 7.5, 7 Hz), 4.67 (1H, m), 4.96 (2H, d, J=48 Hz), 5.19 (1H, ddd, J=10.5, 9.5, 6 Hz), 5.82 (1H, s), 6.36 (1H, m), 7.00 (1H, ddd, J=16, 7, 7 Hz), 7.15 (1H, d, J=10 Hz), 7.17-7.32 (5H, m), 7.50 (1H, d, J=10.5 Hz);

MASS (ES−) m/e 527;

[α]_{D}²³=−90.7 (c=0.25, CHCl₃).

### EXAMPLE 145

To a stirred solution of dimethyl 3-fluoro-2-oxopropylphosphonate (356 mg) in 2-propanol (10 ml) was added cesium carbonate (599 mg) at 0° C. and the mixture was stirred at ambient temperature for half an hour. To the resulting yellow brown solution was added a solution of the starting compound (Compound (81)) (484 mg) in tetrahydrofuran (5 ml) and 2-propanol (5 ml) at the same temperature and the resulting mixture was stirred for half an hour at the same temperature. The reaction mixture was quenched with 10% aqueous solution of citric acid, diluted with ethyl acetate and water. The organic layer was separated and washed with water and brine. The organic layer was dried over sodium sulfate, filtered and evaporated under reduced pressure. The residue was purified by preparative thin layer chromatography using ethyl acetate to give the Compound E145 (320 mg) as a white amorphous solid.

¹H-NMR (300 MHz, CDCl₃, δ): 0.85 (3H, t, J=7.3 Hz), 1.29 (3H, s), 1.40-1.90 (6H, m), 2.08-2.40 (6H, m), 2.89 (1H, dd, J=14, 6 Hz), 3.18 (1H, dd, J=14, 10 Hz), 3.26 (1H, m), 3.77 (3H, s), 3.86 (1H, m), 4.22 (1H, dt, J=10, 7.5 Hz), 4.67 (1H, m), 4.96 (2H, d, J=47 Hz), 5.14 (1H, ddd, J=10, 10, 6 Hz), 5.93 (1H, s), 6.36 (1H, m), 6.81 (2×1H, d, J=8.5 Hz), 7.00 (1H, dt, J=16, 7 Hz), 7.14 (2×1H, d, J=8.5 Hz), 7.18 (1H, d, J=10 Hz), 7.49 (1H, d, J=10 Hz);

MASS (ES−) m/e 557;

[α]_{D}³⁰=−108.6° (c=0.16, CHCl₃).

### EXAMPLE 146

Compound E146 was obtained in a manner similar to Example 4.

¹H-NMR (300 MHz, CDCl₃, δ): 0.84 (3H, t, J=7 Hz), 1.23-1.42 (4H, m), 1.28 (3H, S), 1.53-1.90 (6H, m), 2.07-2.24 (2H, m), 2.25-2.45 (2H, m), 2.54 (2H, m), 2.89 (1H, dd, J=13.5, 6.5 Hz), 3.18 (1H, dd, J=13.5, 9.5 Hz), 3.26 (1H, m), 3.77 (3H, s), 3.85 (1H, m), 4.19 (1H, dt, J=10.5, 7.5 Hz), 4.67 (1H, m), 4.79 (2H, d, J=48 Hz), 5.13 (1H, ddd, J=10, 9.5, 6.5 Hz), 5.80 (1H, s), 6.81 (2×1H, d, J=8.5 Hz), 7.10 (1H, d, J=10.5 Hz), 7.14 (2×1H, d, J=8.5 Hz), 7.53 (1H, d, J=10 Hz);

MASS (ES−) m/e 559;

[α]_{D}³⁰=−118.8° (c=0.40, CHCl₃).

### EXAMPLE 147

To a stirred solution of the starting compound E146 (55 mg) in ethanol (4 ml) was added a solution of sodium borohydride in ethanol at 0° C. and stirred at ambient temperature for half an hour. The reaction was quenched with water and most of the solvent was removed under reduced pressure. The residue was diluted with ethyl acetate and washed with brine. The organic layer was dried over sodium sulfate, filtered and evaporated under reduced pressure. The resiidue was purified by preparative thin layer chromatography using 50% ethyl acetate/hexane (v/v) as an eluting solvent mixture to give an amorphous, which was dissolved in tert-butyl alcohol and lyophilized to give Compound E147 (49 mg).

¹H-NMR (300 MHz, CDCl₃, δ): 0.84 (3H, t, J=7.3 Hz), 1.22-1.53 (8H, m), 1.28 (3H, s), 1.56-1.90 (4H, m), 2.07-2.24 (3H, m), 2.25-2.40 (2H, m), 2.89 (1H, dd, J=14, 6 Hz), 3.18 (1H, dd, J=14, 9.5 Hz), 3.26 (1H, m), 3.77 (3H, s), 3.80-3.94 (2H, m), 4.20 (1H, m), 4.27 (1H, m), 4.41 (1H, ddd, J=47, 9.5, 3 Hz), 4.67 (1H, m), 5.13 (1H, ddd, J=10, 9.5, 6 Hz), 5.94 (1H, d, J=5 Hz), 6.81 (2×1H, d, J=8.5 Hz), 7.11 (1H, d, J=10 Hz), 7.14 (2×1H, d, J=8.5 Hz), 7.54 (1H, d, J=10 Hz);

MASS (ES−): m/e 561;

[α]_{D}²⁴=−107.5° (c=0.21, CHCl₃).

### EXAMPLE 148

To a stirred solution of Compound E138 (165 mg) in dichrolomethane (5 ml) was added a solution of diethylaminosulfurtrifluoride (71.7 mg) in dichloromethane at ambient temperature. The reaction mixture was stirred at the same temperature for three days. The solvent was removed under reduced pressure and the residue was dissolved in ethyl acetate. The organic layer was washed with saturated sodium bicarbonate solution, water and brine. The organic layer was dried over sodium sulfate, filtered and evaporated under reduced pressure. The crude product was purified by preparative thin layer chromatography using 50% ethyl acetate/hexane (v/v) as a solvent mixture to give the Compound E148 (136 mg) as a white amorphous solid.

¹H-NMR (300 MHz, CDCl₃, δ): 0.84 (3H, t, J=7.3 Hz), 0.88 (3H, d, J=6.5 Hz), 1.22-1.44 (4H, m), 1.29 (3H, s), 1.47 (3H, dd, J=24, 7 Hz), 1.52-1.68 (3H, m), 1.82 (1H, m), 2.14 (1H, dq, J=14, 7.3 Hz), 2.26-2.43 (2H, m), 2.56-2.68 (3H, m), 2.72 (1H, dd, J=9.5, 8 Hz), 2.96 (1H, dd, J=13.5, 6 Hz), 3.24 (1H, dd, J=13.5, 10 Hz), 4.06 (1H, dd, J=9.5, 7 Hz), 4.20 (1H, dt, J=10, 7.5 Hz), 4.67 (1H, dd, J=8, 2 Hz), 4.86 (1H, dq, J=50, 7 Hz), 5.16 (1H, ddd, J=10, 10, 6 Hz), 5.84 (1H, s), 7.16 (1H, d, J=10 Hz), 7.16-7.31 (5H, m), 7.54 (1H, d, J=10 Hz);

MASS (ES−): m/e 557;

[α]_{D}²⁵=−100.4° (c=0.26, CHCl₃).

### EXAMPLE 149

To a stirred solution of the Compound E142 (99 mg) in tetrahydrofuran (4 ml) was added n-butyllithium (1.50 M in hexane, 0.60 ml) dropwise at −78° C. The mixture was stirred at the same temperature for twenty-five minutes. The reaction mixture was quenched with water at the same temperature and the resulting mixture was extracted with ethyl acetate. The organic layer was washed with brine, dried over sodium sulfate, filtered and evaporated under reduced pressure. The residue was purified by preparative thin layer chromatography using 50% ethyl acetate/hexane (v/v) as a solvent mixture to give Compound E149 (38 mg) as a white amorphous solid.

¹H-NMR (300 MHz, CDCl₃, δ): 0.80-0.96 (9H, m), 1.20-1.44 (6H, m), 1.29 (3H, S), 1.48-1.69 (5H, m), 1.81 (1H, m), 2.16 (1H, m), 2.26-2.42 (5H, m), 2.63 (1H, m), 2.72 (1H, m), 2.96 (1H, m), 3.25 (1H, m), 4.06 (1H, m), 4.19 (1H, m), 4.67 (1H, brd, J=8 Hz), 5.16 (1H, m), 5.79 (1H, S), 7.14 (1H, d, J=10 Hz), 7.18-7.32 (5H, m), 7.54 (1H, d, J=10 Hz);

MASS (ES+): m/e 569;

[α]_{D}²¹=−116.2° (c=0.18, CHCl₃).

### EXAMPLE 150

Compound E150 was obtained from the Compound (297) in a manner similar to Example 1.

¹H-NMR (300 MHz, CDCl₃, δ): 0.85 (3H, t, J=7.3 Hz), 1.09 (9H, s), 1.23 (3H, d, J=7.0 Hz), 1.29 (3H, s), 1.36-1.92 (8H, m), 2.08-2.41 (4H, m), 3.01 (1H, dd, J=13.6, 6.2 Hz), 3.22-3.33 (1H, m), 3.31 (1H, dd, J=13.6, 9.9 Hz), 3.83-3.92 (1H, m), 4.16-4.31 (1H, m), 4.27 (1H, q, J=7.0 Hz), 4.63-4.70 (1H, m), 5.23 (1H, ddd, J=10.6, 9.9, 6.2 Hz), 5.87 (1H, s), 6.62 (1H, d, J=15.8 Hz), 6.87 (1H, dt, J=15.8, 6.6 Hz), 7.14 (1H, d, J=10.3 Hz), 7.19-7.25 (1H, m), 7.31-7.49 (8H, m), 7.54-7.78 (7H, m), 7.90 (2H, d, J=8.1 Hz), 8.65-8.69 (1H, m);

MASS (ES+): m/e 856.53 (M+1).

### EXAMPLE 151

Compound E151 was obtained from the Compound E150 in a manner similar to Example 3.

¹H-NMR (300 MHz, CDCl₃, δ): 0.85 (3H, t, J=7.3 Hz), 1.11 (9H, s), 1.23 (3H, d, J=7.0 Hz), 1.29 (3H, s), 1.36-1.89 (8H, m), 2.10-2.26 (2H, m), 2.26-2.40 (2H, m), 2.47-2.56 (2H, m), 2.96-3.06 (1H, m), 3.23-3.37 (2H, m), 3.81-3.94 (1H, m), 4.09-4.31 (2H, m), 4.64-4.71 (1H, m), 5.17-5.30, (1H, m), 5.87 (1H, s), 7.08 (1H, d, J=10.3 Hz), 7.25-7.34 (1H, m), 7.34-7.51 (8H, m), 7.58-7.88 (7H, m), 7.89-7.99 (2H, m), 8.67-8.77 (1H, m);

MASS (ES+): m/e 858.45 (M+1).

### EXAMPLE 152

Compound E152 was obtained from the Compound E151 in a manner similar to Example 6.

¹H-NMR (300 MHz, CDCl₃, δ): 0.85 (3H, t, J=7.3 Hz), 1.18-1.42 (2H, m), 1.29 (3H, s), 1.38 (3H, d, J=7.3 Hz), 1.47-1.90 (8H, m), 2.06-2.59 (6H, m), 3.01 (1H, dd, J=13.6, 9.5 Hz), 3.21-3.34 (1H, m), 3.31 (1H, dd, J=13.6, 5.5 Hz), 3.56 (1H, d, J=4.4 Hz), 3.81-3.93 (1H, m), 4.15-4.29 (2H, m), 4.62-4.71 (1H, m), 5.23 (1H, ddd, J=10.6, 9.5, 5.5 Hz), 5.88 (1H, s), 7.11 (1H, d, J=9.9 Hz), 7.18-7.25 (1H, m), 7.35 (2H, d, J=8.4 Hz), 7.59 (1H, d, J=10.6 Hz), 7.66-7.79 (2H, m), 7.90 (2H, d, J=8.4 Hz), 8.64-8.71 (1H, m);

MASS (ES−): m/e 618.20 (M−1).

### EXAMPLE 153

Compound E153 was obtained from the Compound (300) in a manner similar to Example 1.

¹H-NMR (300 MHz, CDCl₃, δ): 0.84 (3H, t, J=7.3 Hz), 1.11 (9H, s), 1.22 (3H, d, J=7.0 Hz), 1.30 (3H, s), 1.37-1.92 (8H, m), 2.08-2.40 (4H, m), 3.06 (1H, dd, J=13.9, 6.6 Hz), 3.27-3.39 (1H, m), 3.30 (1H, dd, J=13.9, 9.2 Hz), 3.85-3.95 (1H, m), 4.18-4.32 (1H, m), 4.26 (1H, q, J=7.0 Hz), 4.67-4.73 (1H, m), 5.18-5.29 (1H, m), 5.93 (1H, s), 6.63 (1H, d, J=15.4 Hz), 6.88 (1H, dt, J=15.4, 6.6 Hz), 7.11 (1H, d, J=10.3 Hz), 7.32-7.52 (8H, m), 7.50 (2H, dd, J=4.4, 1.8 Hz), 7.55-7.70 (7H, m), 8.65 (2H, dd, J=4.4, 1.8 Hz);

MASS (ES+): m/e 856.38 (M+1).

### EXAMPLE 154

Compound E154 was obtained from the Compound E153 in a manner similar to Example 3.

¹H-NMR (300 MHz, CDCl₃, δ): 0.84 (3H, t), 1.11 (9H, s), 1.20 (3H, d, J=7.0 Hz), 1.30 (3H, s), 1.36-1.90 (10H, m), 2.12-2.40 (4H, m), 2.48-2.56 (1H, m), 3.00-3.10 (1H, m), 3.24-3.39 (1H, m), 3.83-3.94 (1H, m), 4.13-4.29 (1H, m), 4.20 (1H, q, J=7.0 Hz), 4.67-4.73 (1H, m), 5.19-5.29 (1H, m), 5.92 (1H, s), 7.06 (1H, d, J=9.9 Hz), 7.33-7.53 (10H, m), 7.54-7.76 (7H, m), 8.62-8.68 (2H, m);

MASS (ES+): m/e 858.39 (M+1).

### EXAMPLE 155

Compound E155 was obtained from the Compound E154 in a manner similar to Example 6.

¹H-NMR (300 MHz, CDCl₃, δ): 0.83 (3H, t, J=7.3 Hz), 1.17-1.43 (2H, m), 1.29 (3H, s), 1.38 (3H, d, J=7.0 Hz), 1.46-2.58 (12H, m), 3.04 (1H, dd, J=13.9, 6.6 Hz), 3.26-3.42 (1H, m), 3.29 (1H, dd, J=13.9, 9.2 Hz), 3.62 (1H, brs), 3.80-3.94 (1H, m), 4.14-4.30 (2H, m), 4.62-4.74 (1H, m), 5.23 (1H, ddd, J=10.3, 9.2, 6.6 Hz), 5.97 (1H, s), 7.08 (1H, d, J=10.3 Hz), 7.36 (2H, d, J=8.1 Hz), 7.45-7.53 (2H, m), 7.57 (2H, d, J=8.1 Hz), 7.64 (1H, d, J=10.3 Hz), 8.60-8.68 (2H, m);

MASS (ES+): m/e 620.20 (M+1).

### EXAMPLE 156

Compound E156 was obtained from the Compound (305) in a manner similar to Example 1.

¹H-NMR (300 MHz, CDCl₃, δ): 0.83 (3H, t, J=7.3 Hz), 1.09 (9H, s), 1.22 (3H, d, J=7.0 Hz), 1.29 (3H, s), 1.35-1.70 (6H, m), 1.72-1.89 (2H, m), 2.06-2.37 (4H, m), 2.58 (2H, t, J=7.6 Hz), 2.88-2.96 (2H, m), 2.95 (1H, dd, J=13.6, 6.2 Hz), 3.20 (1H, dd, J=13.6, 9.9 Hz), 3.25-3.38 (3H, m), 3.47-3.54 (2H, m), 3.56-3.66 (4H, m), 3.83-3.92 (1H, m), 4.17-4.27 (1H, m), 4.27 (1H, q, J=7.0 Hz), 5.17 (1H, ddd, J=9.9, 9.9, 6.2 Hz), 5.89 (1H, s), 6.61 (1H, d, J=15.8 Hz), 6.87 (1H, dt, J=15.8, 6.6 Hz), 7.07-7.19 (1H, m), 7.11 (2H, d, J=8.4 Hz), 7.16 (2H, d, J=8.4 Hz), 7.31-7.47 (6H, m), 7.54 (1H, d, J=10.3 Hz), 7.59 (1H, d, J=8.1 Hz), 7.59 (1H, d, J=7.7 Hz), 7.65 (1H, d, J=8.1 Hz), 7.65 (1H, d, J=7.7 Hz);

MASS (ES+): m/e 920.48 (M+1).

### EXAMPLE 157

Compound E157 was obtained from the Compound E156 in a manner similar to Example 3.

¹H-NMR (300 MHz, CDCl₃, δ): 0.83 (3H, t, J=7.3 Hz), 1.10 (9H, s), 1.17-1.32 (2H, m), 1.18 (3H, d, J=7.0 Hz), 1.28 (3H, s), 1.38-1.90 (8H, m), 2.04-2.20 (4H, m), 2.46-2.54 (2H, m), 2.58 (2H, t, J=8.7 Hz), 2.88-3.00 (1H, m), 2.93 (2H, t, J=8.7 Hz), 3.21 (1H, dd, J=14.1, 9.0 Hz), 3.28-3.38 (3H, m), 3.48-3.56 (2H, m), 3.61 (4H, s), 3.78-3.96 (1H, m), 4.10-4.27 (2H, m), 4.62-4.70 (1H, m), 5.10-5.22 (1H, m), 5.86 (1H, s), 7.06 (1H, d, J=9.9 Hz), 7.08-7.18 (4H, m), 7.32-7.51 (6H, m), 7.52-7.78 (5H, m);

MASS (ES+): m/e 922.45 (M+1).

### EXAMPLE 158

Compound E158 was obtained from the Compound E157 in a manner similar to Example 6.

¹H-NMR (300 MHz, CDCl₃, δ): 0.83 (3H, t, J=7.3 Hz), 1.19-1.41 (2H, m), 1.28 (3H, s), 1.38 (3H, d, J=7.3 Hz), 1.52-1.89 (8H, m), 2.06-2.40 (4H, m), 2.46 (2H, dt, J=11.7, 7.3 Hz), 2.58 (2H, t, J=7.7 Hz), 2.88-2.96, (2H, m), 2.95 (1H, dd, J=13.6, 6.2 Hz), 3.20 (1H, dd, J=13.6, 9.5 Hz), 3.24-3.40 (3H, m), 3.43-3.54 (2H, m), 3.54-3.67 (4H, m), 3.87 (1H, dt, J=8.1, 4.8 Hz), 4.14-4.29 (2H, m), 4.64-4.72 (1H, m), 5.17 (1H, ddd, J=10.6, 9.5, 6.2 Hz), 5.92 (1H, s), 7.05-7.19 (5H, m), 7.56 (1H, d, J=10.3 Hz);

MASS (ES+): m/e 684.54 (M+1).

### EXAMPLE 159

Compound E159 was obtained from the Compound (308) in a manner similar to Example 1.

¹H-NMR (300 MHz, CDCl₃, δ): 0.83 (3H, t, J=7.3 Hz), 1.10 (9H, s), 1.25 (3H, d, J=7.0 Hz), 1.28 (12H, s), 1.38-1.61 (6H, m), 1.71-1.88 (2H, m), 2.09-2.38 (4H, m), 2.34 (2H, t, J=7.3 Hz), 2.89 (2H, t, J=7.3 Hz), 2.93 (1H, dd, J=13.5, 6.2 Hz), 3.21 (1H, dd, J=13.5, 9.2 Hz), 3.25-3.37 (1H, m), 3.83-3.91 (1H, m), 4.16-4.25 (1H, m), 4.28 (1H, q, J=7.0 Hz), 4.64-4.69 (1H, m), 5.07-5.19 (2H, m), 5.87 (1H, s), 6.61 (1H, d, J=15.8 Hz), 6.87 (1H, dt, J=15.8, 6.6 Hz), 7.07-7.17 (5H, m), 7.31-7.49 (6H, m), 7.53 (1H, d, J=9.9 Hz), 7.57-7.68 (4H, m);

MASS (ES+): m/e 906.30 (M+1).

### EXAMPLE 160

Compound E160 was obtained from the Compound E159 in a manner similar to Example 3.

¹H-NMR (300 MHz, CDCl₃, δ): 0.83 (3H, t, J=7.3 Hz), 1.10 (9H, s), 1.15-1.34 (2H, m), 1.25 (2H, d, J=7.0 Hz), 1.28 (12H, s), 1.39-1.88 (8H, m), 2.09-2.39 (4H, m), 2.35 (2H, t, J=7.7 Hz), 2.47-2.61 (2H, m), 2.89 (2H, t, J=7.7 Hz), 2.92 (1H, dd, J=13.9, 6.6 Hz), 3.20 (1H, dd, J=13.9, 9.9 Hz), 3.23-3.38 (1H, m), 3.81-3.91 (1H, m), 4.12-4.30 (2H, m), 4.64-4.70 (1H, m), 5.10 (1H, s), 5.16 (1H, ddd, J=10.3, 9.9, 6.6 Hz), 5.85 (1H, s), 7.07-7.18 (5H, m), 7.07 (1H, d, J=10.3 Hz), 7.33-7.49 (6H, m), 7.57 (1H, d, J=9.9 Hz), 7.59-7.68 (4H, m);

MASS (ES+): m/e 908.49 (M+1).

### EXAMPLE 161

Compound E161 was obtained from the Compound E160 in a manner similar to Example 6.

¹H-NMR (300 MHz, CDCl₃, δ): 0.84 (3H, t, J=7.3 Hz), 1.14-1.40 (2H, m), 1.28 (9H, s), 1.38 (3H, d, J=7.0 Hz), 1.45-1.70 (6H, m), 1.71-1.88 (2H, m), 2.07-2.41 (4H, m), 2.34 (2H, t, J=7.7 Hz), 2.46 (2H, dt, J=11.7, 7.3 Hz), 2.88 (2H, t, J=7.7 Hz), 2.92 (1H, dd, J=13.9, 5.9 Hz), 3.20 (1H, dd, J=13.9, 9.5 Hz), 3.22-3.33 (1H, m), 3.55 (1H, d, J=4.4 Hz), 3.80-3.92 (1H, m), 4.14-4.29 (2H, m), 4.65-4.71 (1H, m), 5.10 (1H, s), 5.16 (1H, ddd, J=10.3, 9.5, 5.9 Hz), 5.89 (1H, s), 7.09 (2H, d, J=8.1 Hz), 7.09-7.16 (1H, m), 7.14 (2H, d, J=8.1 Hz), 7.55 (1H, d, J=10.3 Hz);

MASS (ES+): m/e 670.53 (M+1).

### EXAMPLE 162

To a solution of the Compound E138 (500 mg) in pyridine (1.2 ml) was added benzyl chloride (0.109 ml) under ice-cooling and the mixture was stirred for 5 hours under ambient temperature. To the reaction mixture was added an additional benzyl chloride (0.2 equivalent per Compound E138) and the mixture was stirred for 2 hours. To the mixture was added ice-cooled 1N hydrochloric acid. The mixture was stirred for 10 min, extracted with ethyl acetate, and the extract was washed with water, saturated aqueous sodium bicarbonate solution, water and saturated brine, and dried over sodium sulfate. The obtained crude compound was purified by column chromatography (eluting with hexane/ethyl acetate=2/1 then 1/1) to give the objective Compound E162. Since the obtained Compound E162 included small amount of pyridine, the compound was dissolved into ethyl acetate, washed with 1N hydrochloric acid twice, then washed with saturated aqueous sodium bicarbonate solution, water and saturated brine, and dried over sodium sulfate. The mixture was filtered and the filtrate was evaporated to give purified Compound E162.

¹H-NMR (300 MHz, CDCl₃, δ): 0.83 (3H, t, J=7.3 Hz), 0.88 (3H, d, J=6.6 Hz), 1.22-1.42 (4H, m), 1.48-1.88 (4H, m), 1.53 (3H, d, J=7.0 Hz), 2.07-2.21 (2H, m), 2.24-2.77 (7H, m), 2.95 (1H, dd, J=13.6, 5.9 Hz), 3.24 (1H, dd, J=13.6, 9.9 Hz), 4.05 (2H, dd, J=9.5, 7.3 Hz), 4.20 (1H, dt, J=10.3, 7.3 Hz), 4.67 (1H, dd, J=8.1, 2.2 Hz), 5.16 (1H, ddd, J=10.3, 9.9, 5.9 Hz), 5.33 (1H, q, J=7.0 Hz), 5.87 (1H, s), 7.15 (1H, d, J=10.3 Hz), 7.19-7.31 (5H, m), 7.44-7.51 (2H, m), 7.56 (1H, d, J=10.3 Hz), 7.56-7.63 (1H, m), 8.08 (2H, d, J=8.4 Hz);

MASS (ES+): m/e 661.51 (M+1).

### EXAMPLE 163

Compound E163 was obtained from the Compound E162 in a manner similar to Preparation 307.

¹H-NMR (300 MHz, CDCl₃, δ): 0.85 (3H, t, J=7.3 Hz), 1.04 (3H, d, J=7.0 Hz), 1.16-1.40 (4H, m), 1.41-1.85 (4H, m), 1.52 (3H, d, J=7.0 Hz), 1.96-2.12 (1H, m), 2.27-2.79 (8H, m), 3.10-3.23 (2H, m), 4.13 (2H, dd, J=9.9, 7.7 Hz), 4.21 (1H, dt, J=10.3, 7.3 Hz), 4.69-4.77 (1H, m), 5.17-5.29 (1H, m), 5.32 (1H, q, J=7.0 Hz), 6.03 (1H, s), 7.09 (1H, d, J=10.6 Hz), 7.43-7.51 (2H, m), 7.56-7.63 (1H, m), 7.63 (1H, d, J=10.3 Hz), 8.08 (2H, d, J=8.4 Hz);

MASS (ES−): m/e 627.53 (M−1).

### EXAMPLE 164

Compound E164 was obtained from the Compound E163 in a manner similar to Preparation 308.

¹H-NMR (300 MHz, CDCl₃, δ): 0.85 (3H, t, J=7.3 Hz), 0.88 (3H, t, J=7.0 Hz), 1.06 (3H, d, J=6.6 Hz), 1.18-1.86 (12H, m), 1.28 (3H, s), 1.52 (3H, d, J=7.0 Hz), 1.97-2.12 (1H, m), 2.26-2.79 (8H, m), 2.98 (1H, dd, J=15.4, 11.0 Hz), 3.13-3.29 (4H, m), 4.16-4.27 (1H, m), 4.21 (1H, dt, J=10.3, 7.7 Hz), 4.72 (1H, d, J=7.7 Hz), 5.28-5.39 (1H, m), 5.32 (1H, q, J=7.0 Hz), 5.47-5.56 (1H, m), 5.88 (1H, s), 7.13 (1H, d, J=10.3 Hz), 7.39-7.53 (3H, m), 7.56-7.64 (1H, m), 8.08 (2H, d, J=7.7 Hz);

MASS (ES+): m/e 698.56 (M+1).

### EXAMPLE 165

Compound E165 was obtained from the Compound E164 in a manner similar to Preparation 77.

¹H-NMR (300 MHz, CDCl₃, δ): 0.86 (3H, t, J=7.3 Hz), 0.88 (3H, t, J=7.0 Hz), 1.07 (3H, d, J=6.6 Hz), 1.18-1.68 (10H, m), 1.30 (3H, s), 1.38 (3H, d, J=7.3 Hz), 1.72-1.85 (1H, m), 1.98-2.13 (1H, m), 2.28-2.55 (8H, m), 2.64-2.79 (1H, m), 2.99 (1H, dd, J=14.7, 11.0 Hz), 3.15-3.28 (3H, m), 3.45-3.56 (1H, m), 4.13-4.28 (3H, m), 4.73 (1H, d, J=7.7 Hz), 5.34 (1H, ddd, J=11.0, 9.9, 4.4 Hz), 5.45-5.53 (1H, m), 5.87 (1H, s), 7.16 (1H, d, J=10.3 Hz), 7.40 (1H, d, J=9.9 Hz);

MASS (ES+): m/e 594.57 (M+1).

### EXAMPLE 166

Compound E166 was obtained from the Compound (312) in a manner similar to Example 1.

¹H-NMR (300 MHz, CDCl₃, δ): 0.86 (3H, t, J=7.3 Hz), 1.09 (9H, s), 1.22 (3H, d, J=6.6 Hz), 1.31 (3H, s), 1.37-2.42 (12H, m), 2.71 (1H, dd, J=15.4, 4.8 Hz), 3.20 (1H, dd, J=15.4, 10.6 Hz), 3.74-3.87 (1H, m), 3.87-3.98 (1H, m), 4.18-4.31 (1H, m), 4.27 (1H, q, J=6.6 Hz), 4.68-4.74 (1H, m), 5.44 (1H, ddd, J=11.0, 10.6, 4.8 Hz), 5.97 (1H, s), 6.61 (1H, d, J=15.8 Hz), 6.87 (1H, dt, J=15.8, 6.6 Hz), 7.05-7.15 (2H, m), 7.22-7.53 (10H, m), 7.49 (1H, d, J=10.6 Hz), 7.56-7.75 (5H, m);

MASS (ES+): m/e 822.46 (M+1).

### EXAMPLE 167

Compound E167 was obtained from the Compound E166 in a manner similar to Example 3.

¹H-NMR (300 MHz, CDCl₃, δ): 0.86 (3H, t, J=7.3 Hz), 1.10 (9H, s), 1.13-1.35 (4H, m), 1.18 (3H, d, J=6.6 Hz), 1.31 (3H, s), 1.38-2.45 (8H, m), 2.46-2.55 (2H, m), 2.72 (1H, dd, J=15.8, 4.4 Hz), 3.21 (1H, dd, J=15.8, 11.0 Hz), 3.76-3.87 (1H, m), 3.87-3.99 (1H, m), 4.07-4.27 (1H, m), 4.19 (1H, q, J=6.6 Hz), 4.67-4.74 (1H, m), 5.45 (1H, ddd, J=11.0, 10.6, 4.4 Hz), 5.96 (1H, s), 7.04 (1H, d, J=10.3 Hz), 7.09 (1H, dd, J=7.7, 7.3 Hz), 7.25-7.51 (10H, m), 7.54 (1H, d, J=10.6 Hz), 7.59-7.67 (4H, m), 7.70-7.75 (1H, m);

MASS (ES+): m/e 824.55 (M+1).

### EXAMPLE 168

Compound E168 was obtained from the Compound E167 in a manner similar to Example 6.

¹H-NMR (300 MHz, CDCl₃, δ): 0.86 (3H, t, J=7.3 Hz), 1.15-1.44 (4H, m), 1.31 (3H, s), 1.38 (3H, d, J=7.3 Hz), 1.46-2.57 (12H, m), 2.72 (1H, dd, J=15.4, 4.4 Hz), 3.20 (1H, dd, J=15.4, 11.0 Hz), 3.56 (1H, d, J=4.8 Hz), 3.81 (1H, dt, J=10.3, 7.7 Hz), 3.87-3.98 (1H, m), 4.16-4.28 (2H, m), 4.68-4.75 (1H, m), 5.44 (1H, ddd, J=11.0, 10.3, 4.4 Hz), 5.97 (1H, s), 7.03-7.15 (1H, m), 7.07 (1H, d, J=9.9 Hz), 7.24-7.35 (2H, m), 7.37-7.47 (3H, m), 7.51 (1H, d, J=10.3 Hz);

MASS (ES+): m/e 586.30 (M+1).

### EXAMPLE 169

Compound E169 was obtained from the Compound (315) in a manner similar to Example 1.

¹H-NMR (300 MHz, CDCl₃, b): 0.84 (3H, t, J=7.3 Hz), 1.10 (9H, s), 1.28 (3H, s), 1.29 (3H, d, J=7.0 Hz), 1.38-1.92 (8H, m), 2.09-2.39 (4H, m), 2.95 (1H, dd, J=13.9, 6.6 Hz), 3.21 (1H, dd, J=13.9, 9.2 Hz), 3.22-3.35 (1H, m), 3.80-3.92 (1H, m), 4.15-4.33 (1H, m), 4.28 (1H, q, J=7.0 Hz), 4.59 (2H, s), 4.64-4.72 (1H, m), 5.15 (1H, ddd, J=10.3, 9.2, 6.6 Hz), 5.88 (1H, s), 6.62 (1H, d, J=15.8 Hz), 6.85 (1H, dt, J=15.8, 7.0 Hz), 6.92 (2H, d, J=8.8 Hz), 7.11 (1H, d, J=10.3 Hz), 7.16 (1H, dd, J=7.7, 7.7 Hz), 7.22 (2H, d, J=8.8 Hz), 7.30-7.50 (8H, m), 7.51-7.70 (7H, m), 8.25 (1H, brs);

MASS (ES+): m/e 928.43 (M+1).

### EXAMPLE 170

Compound E170 was obtained from the Compound E169 in a manner similar to Example 3.

¹H-NMR (300 MHz, CDCl₃, δ): 0.83 (3H, t, J=7.3 Hz), 1.10 (9H, s), 1.15-1.33 (2H, m), 1.25 (3H, d, J=7.0 Hz), 1.28 (3H, s), 1.37-1.86 (8H, m), 2.10-2.38 (4H, m), 2.47-2.54 (2H, m), 2.93 (1H, dd, J=13.9, 6.2 Hz), 3.18-3.33 (1H, m), 3.20 (1H, dd, J=13.9, 9.2 Hz), 3.80-3.90 (1H, m), 4.12-4.30 (2H, m), 4.58 (2H, s), 4.65-4.71 (1H, m), 5.14 (1H, ddd, J=10.3, 9.2, 6.2 Hz), 5.88 (1H, s), 6.91 (2H, d, J=8.4 Hz), 7.05 (1H, d, J=9.9 Hz), 7.16 (1H, dd, J=7.3, 7.3 Hz), 7.21 (2H, d, J=8.4 Hz), 7.32-7.51 (8H, m), 7.54-7.71 (7H, m), 8.24 (1H, brs);

MASS (ES+): m/e 930.41 (M+1).

### EXAMPLE 171

Compound E171 was obtained from the Compound E170 in a manner similar to Example 6.

¹H-NMR (300 MHz, CDCl₃, δ): 0.83 (3H, t, J=7.3 Hz), 1.22-1.41 (4H, m), 1.28 (3H, s), 1.38 (3H, d, J=7.0 Hz), 1.49-11.89 (6H, m), 2.08-2.58 (6H, m), 2.93 (1H, dd, J=13.6, 6.2 Hz), 3.19 (1H, dd, J=13.6, 9.2 Hz), 3.22-3.33 (1H, m), 3.55° (1H, d, J=4.8 Hz), 3.81-3.91 (1H, m), 4.14-4.28 (2H, m), 4.58 (2H, s), 4.65-4.71 (1H, m), 5.15 (1H, ddd, J=10.3, 9.2, 6.2 Hz), 5.88 (1H, s), 6.91 (2H, d, J=8.8 Hz), 7.08 (1H, d, J=10.3 Hz), 7.16 (1H, dd, J=7.3, 7.3 Hz), 7.21 (2H, d, J=8.8 Hz), 7.36 (2H, dd, J=7.3, 7.3 Hz), 7.56 (1H, d, J=10.3 Hz), 7.57 (2H, d, J=7.3 Hz), 8.24 (1H, brs);

MASS (ES+): m/e 692.37 (M+1).

### EXAMPLE 172

Compound E172 was obtained from the Compound (318) in a manner similar to Example 1.

¹H-NMR (300 MHz, CDCl₃, δ): 0.83 (3H, t, J=7.3 Hz), 0.89 (3H, t, J=7.0 Hz), 1.09 (9H, s), 1.15-1.38 (4H, m), 1.22 (3H, d, J=7.0 Hz), 1.28 (3H, s), 1.38-1.91 (8H, m), 2.09-2.38 (4H, m), 2.87-2.96 (1H, dd, J=13.6, 6.2 Hz), 3.18 (1H, dd, J=13.6, 9.5 Hz), 3.21-3.37 (3H, m), 3.81-3.91 (1H, m), 4.16-4.28 (1H, m), 4.27 (1H, q, J=7.0 Hz), 4.45 (2H, s), 4.64-4.71 (1H, m), 5.13 (1H, ddd, J=10.3, 9.5, 6.2 Hz), 5.91 (1H, s), 6.55 (1H, br), 6.61 (1H, d, J=15.8 Hz), 6.83 (2H, d, J=8.8 Hz), 6.86 (1H, dt, J=15.8, 6.6 Hz), 7.12 (1H, d, J=10.3 Hz), 7.18 (2H, d, J=8.8 Hz), 7.31-7.47 (6H, m), 7.55 (1H, d, J=10.3 Hz), 7.56-7.62 (2H, m), 7.62-7.68 (2H, m);

MASS (ES+): m/e 922.52 (M+1).

### EXAMPLE 173

Compound E173 was obtained from the Compound E172 in a manner similar to Example 3.

¹H-NMR (300 MHz, CDCl₃, δ): 0.83 (3H, t, J=7.3 Hz), 0.89 (3H, t, J=7.0 Hz), 1.00-1.89 (14H, m), 1.10 (9H, s), 1.19 (3H, d, J=6.6 Hz), 1.28 (3H, s), 2.08-2.39 (4H, m), 2.46-2.56 (2H, m), 2.91 (1H, dd, J=13.6, 6.2 Hz), 3.18 (1H, dd, J=13.6, 9.5 Hz), 3.20-3.38 (3H, m), 3.79-3.91 (1H, m), 4.12-4.24 (1H, m), 4.18 (1H, q, J=7.0 Hz), 4.44 (2H, s), 4.64-4.70 (1H, m), 5.13 (1H, ddd, J=9.9, 9.5, 6.2 Hz), 5.88 (1H, s), 6.55 (1H, br), 6.83 (2H, d, J=8.8 Hz), 7.05 (1H, d, J=9.9 Hz), 7.18 (2H, d, J=8.8 Hz), 7.33-7.48 (6H, m), 7.58 (1H, d, J=9.9 Hz), 7.58-7.69 (4H, m);

MASS (ES+): m/e 924.63 (M+1).

### EXAMPLE 174

Compound E174 was obtained from the Compound E173 in a manner similar to Example 6.

¹H-NMR (300 MHz, CDCl₃, δ): 0.84 (3H, t, J=7.3 Hz), 0.89 (3H, t, J=6.9 Hz), 1.18-1.42 (6H, m), 1.28 (3H, s), 1.38 (3H, d, J=7.0 Hz), 1.44-1.89 (8H, m), 2.07-2.58 (6H, m), 2.92 (1H, dd, J=13.9, 6.6 Hz), 3.18 (1H, dd, J=13.9, 9.5 Hz), 3.20-3.38 (3H, m), 3.46-3.61 (1H, m), 3.80-3.91 (1H, m), 4.14-4.30 (1H, m), 4.44 (2H, s), 4.64-4.72 (1H, m), 5.13 (1H, ddd, J=10.3, 9.5, 6.6 Hz), 5.92 (1H, s), 6.56 (1H, br), 6.83 (2H, d, J=8.4 Hz), 7.09 (1H, d, J=10.3 Hz), 7.18 (2H, d, J=8.4 Hz), 7.57 (1H, d, J=10.3 Hz);

MASS (ES+): m/e 686.60 (M+1).

### EXAMPLE 175

Compound E175 was obtained from the Compound (321) in a manner similar to Example 1.

¹H-NMR (300 MHz, CDCl₃, δ): 0.84 (3H, t, J=7.3 Hz), 1.10 (9H, s), 1.22 (3H, d, J=6.6 Hz), 1.29 (3H, s), 1.38-1.69 (12H, m), 1.72-1.88 (2H, m), 2.11-2.37 (4H, m), 2.89 (1H, dd, J=13.5, 6.2 Hz), 3.18 (1H, dd, J=13.5, 10.2 Hz), 3.19-3.31 (1H, m), 3.41-3.50 (2H, m), 3.51-3.58 (2H, m), 3.81-3.91 (1H, m), 4.17-4.29 (1H, m), 4.27 (1H, q, J=6.6 Hz), 4.64 (1H, s), 4.64-4.69 (1H, m), 5.13 (1H, ddd, J=10.6, 10.2, 6.2 Hz), 5.86 (1H, s), 6.60 (1H, d, J=15.8 Hz), 6.87 (1H, dt, J=15.8, 5.9 Hz), 7.14 (1H, d, J=10.3 Hz), 7.14 (2H, d, J=8.4 Hz), 7.31-7.47 (6H, m), 7.52 (1H, d, J=10.6 Hz), 7.55-7.69 (4H, m);

MASS (ES+): m/e 920.64 (M+1).

### EXAMPLE 176

Compound E176 was obtained from the Compound E175 in a manner similar to Example 3.

¹H-NMR (300 MHz, CDCl₃, δ): 0.84 (3H, t, J=7.3 Hz), 1.10 (9H, s), 1.16-1.33 (2H, m), 1.18 (3H, d, J=7.0 Hz), 1.29 (3H, s), 1.39-1.88 (14H, m), 2.08-2.37 (4H, m), 2.47-2.55 (2H, m), 2.88 (1H, dd, J=13.5, 5.9 Hz), 3.18 (1H, dd, J=13.5, 10.3 Hz), 3.21-3.31 (1H, m), 3.43-3.51 (2H, m), 3.52-3.60 (2H, m), 3.80-3.90 (1H, m), 4.13-4.23 (1H, m), 4.19 (1H, q, J=7.0 Hz), 4.64 (2H, s), 4.64-4.70 (1H, m), 5.12 (1H, ddd, J=10.3, 9.9, 5.9 Hz), 5.85 (1H, s), 6.85 (2H, d, J=8.8 Hz), 7.08 (1H, d, J=10.3 Hz), 7.14 (2H, d, J=8.8 Hz), 7.33-7.49 (6H, m), 7.55 (1H, d, J=9.9 Hz), 7.59-7.68 (4H, m);

MASS (ES+): m/e 922.50 (M+1).

### EXAMPLE 177

Compound E177 was obtained from the Compound E170 in a manner similar to Example 6.

¹H-NMR (300 MHz, CDCl₃, δ): 0.84 (3H, t, J=7.3 Hz), 1.17-1.40 (2H, m), 1.28 (3H, s), 1.38 (3H, d, J=7.3 Hz), 1.44-1.68 (12H, m), 1.72-1.88 (2H, m), 2.05-2.55 (6H, m), 2.88 (1H, dd, J=13.6, 6.6 Hz), 3.18 (1H, dd, J=13.6, 9.9 Hz), 3.20-3.32 (1H, m), 3.42-3.50 (2H, m), 3.51-3.60 (2H, m), 3.80-3.90 (1H, m), 4.12-4.28 (1H, m), 4.64 (2H, s), 4.64-4.70 (1H, m), 5.12 (1H, ddd, J=10.3, 9.9, 6.6 Hz), 5.84 (1H, s), 6.85 (2H, d, J=8.8 Hz), 7.10 (1H, d, J=10.3 Hz), 7.14 (2H, d, J=8.8 Hz), 7.53 (1H, d, J=10.3 Hz);

MASS (ES+): m/e 684.40 (M+1).

### EXAMPLE 178

Compound E178 was obtained from the Compound (324) in a manner similar to Example 1.

¹H-NMR (300 MHz, CDCl₃, δ): 0.84 (3H, t, J=7.3 Hz), 1.10 (9H, s), 1.21 (3H, d, J=7.0 Hz), 1.30 (3H, s), 1.39-1.90 (8H, m), 2.11-2.39 (4H, m), 3.03 (1H, dd, J=13.6, 9.5 Hz), 3.27-3.38 (1H, m), 3.29 (1H, dd, J=13.6, 6.6 Hz), 3.85-3.94 (1H, m), 4.18-4.28 (1H, m), 4.28 (1H, q, J=7.0 Hz), 4.67-4.72 (1H, m), 5.23 (1H, ddd, J=10.3, 9.5, 6.6 Hz), 5.91 (1H, s), 6.62 (1H, d, J=15.8 Hz), 6.87 (1H, dt, J=15.8, 7.0 Hz), 7.11 (1H, d, J=10.3 Hz), 7.30-7.48 (9H, m), 7.51 (2H, d, J=8.4 Hz), 7.56-7.68 (5H, m), 7.85 (1H, ddd, J=8.1, 4.0, 2.2 Hz), 8.58 (1H, dd, J=4.8, 1.5 Hz), 8.82 (1H, d, J=2.2 Hz);

MASS (ES+): m/e 856.41 (M+1).

### EXAMPLE 179

Compound E179 was obtained from the Compound E178 in a manner similar to Example 3.

¹H-NMR (300 MHz, CDCl₃, δ): 0.84 (3H, t, J=7.3 Hz), 1.10 (9H, s), 1.16-1.33 (2H, m), 1.25 (3H, d, J=7.0 Hz), 1.29 (3H, s), 1.37-1.88 (8H, m), 2.12-2.38 (4H, m), 2.48-2.55 (2H, m), 2.99-3.08 (1H, m), 3.24-3.37 (2H, m), 3.84-3.94 (1H, m), 4.15-4.23 (1H, m), 4.26 (1H, q, J=7.0 Hz), 4.67-4.72 (1H, m), 5.16-5.28 (1H, m), 5.90 (1H, s), 7.06 (1H, d, J=10.3 Hz), 7.32-7.48 (9H, m), 7.51 (2H, d, J=8.4 Hz), 7.58-7.67 (5H, m), 7.85 (1H, ddd, J=8.1, 4.8, 2.2 Hz), 8.57 (1H, dd, J=4.8, 1.5 Hz), 8.81-8.83 (1H, m);

MASS (ES+): m/e 858.48 (M+1).

### EXAMPLE 180

Compound E180 was obtained from the Compound E179 in a manner similar to Example 6.

¹H-NMR (300 MHz, CDCl₃, δ): 0.84 (3H, t, J=7.3 Hz), 1.19-1.42 (2H, m), 1.29 (3H, s), 1.38 (3H, d, J=7.0 Hz), 1.53-1.92 (8H, m), 2.07-2.58 (6H, m), 3.04 (1H, dd, J=13.6, 6.2 Hz), 3.21-3.39 (1H, m), 3.29 (1H, dd, J=13.6, 9.2 Hz), 3.57 (1H, brs), 3.83-3.95 (1H, m), 4.15-4.29 (1H, m), 4.67-4.74 (1H, m), 5.23 (1H, ddd, J=10.3, 9.2, 6.2 Hz), 5.95 (1H, s), 7.10 (1H, d, J=10.3 Hz), 7.35 (2H, d, J=8.4 Hz), 7.36 (1H, d, J=8.1 Hz), 7.51 (1H, d, J=8.4 Hz), 7.62 (1H, d, J=10.3 Hz), 7.86 (1H, ddd, J=8.1, 4.0, 1.8 Hz), 8.58 (1H, d, J=4.0 Hz), 8.83 (1H, s);

MASS (ES+): m/e 620.48 (M+1).

### EXAMPLE 181

Compound E181 was obtained from the Compound (333) in a manner similar to Example 1.

¹H-NMR (300 MHz, CDCl₃, δ): 0.83 (3H, t, J=7.3 Hz), 1.10 (9H, s), 1.21 (3H, d, J=7.0 Hz), 1.28 (3H, s), 1.39-1.52 (2H, m), 1.54-1.72 (4H, m), 1.74-1.92 (2H, m), 2.09-2.38 (4H, m), 2.93 (1H, dd, J=13.6, 6.2 Hz), 3.17 (1H, dd, J=13.6, 9.2 Hz), 3.27-3.37 (1H, m), 3.81-3.90 (1H, m), 4.17-4.29 (1H, m), 4.27 (1H, q, J=7.0 Hz), 4.66-4.71 (1H, m), 5.07-5.17 (1H, m), 5.88 (1H, s), 6.61 (1H, d, J=15.7 Hz), 6.89 (1H, dt, J=15.7, 6.6 Hz), 7.04 (1H, d, J=10.6 Hz), 7.07 (1H, dd, J=8.4, 2.6 Hz), 7.30-7.49 (8H, m), 7.53-7.74 (5H, m);

MASS (ES+): m/e 847.51 (M+1).

### EXAMPLE 182

Compound E182 was obtained from the Compound E181 in a manner similar to Example 3.

¹H-NMR (300 MHz, CDCl₃, δ): 0.83 (3H, t, J=7.3 Hz), 1.10 (9H, s), 1.15-1.33 (2H, m), 1.18 (3H, d, J=6.6 Hz), 1.28 (3H, s), 1.38-1.65 (4H, m), 1.71-1.92 (2H, m), 2.09-2.39 (4H, m), 2.51 (2H, t, J=7.3 Hz), 2.93 (1H, dd, J=13.5, 6.6 Hz), 3.17 (1H, dd, J=13.5, 9.2 Hz), 3.27-3.38 (1H, m), 3.79-3.89 (1H, m), 4.14-4.25 (1H, m), 4.19 (1H, q, J=6.6 Hz), 4.65-4.72 (1H, m), 5.12 (1H, dd, J=9.9, 9.2, 6.6 Hz), 5.87 (1H, s), 6.98 (1H, d, J=9.9 Hz), 7.07 (1H, dd, J=8.4, 2.2 Hz), 7.32-7.49 (8H, m), 7.58-7.69 (5H, m);

MASS (ES+): m/e 849.53 (M+1).

### EXAMPLE 183

Compound E183 was obtained from the Compound E182 in a manner similar to Example 6.

¹H-NMR (300 MHz, CDCl₃, δ): 0.83 (3H, t, J=7.3 Hz), 1.17-1.40 (2H, m), 1.28 (3H, s), 1.38 (2H, d, J=7.0 Hz), 1.52-1.72 (4H, m), 1.73-1.93 (2H, m), 2.07-2.57 (6H, m), 2.93 (1H, dd, J=13.9, 6.6 Hz), 3.17 (1H, dd, J=13.9, 9.2 Hz), 3.26-3.37 (1H, m), 3.55 (1H, d, J=4.4 Hz), 3.78-3.89 (1H, m), 4.13-4.29 (2H, m), 4.64-4.72 (1H, m), 5.06-5.17 (1H, m), 5.88 (1H, s), 7.01 (1H, d, J=10.3 Hz), 7.07 (1H, dd, J=8.1, 2.2 Hz), 7.33 (1H, d, J=2.2 Hz), 7.35 (1H, d, J=8.1 Hz), 7.60 (1H, d, J=10.3 Hz);

MASS (ES+): m/e 611.35 (M+1).

### EXAMPLE 184

Compound E184 was obtained from the Compound (341) in a manner similar to Example 1.

¹H-NMR (300 MHz, CDCl₃, δ): 0.78 (3H, t, J=7.3 Hz), 1.10 (9H, s), 1.23 (3H, d, J=7.0 Hz), 1.28 (3H, s), 1.38-1.94 (6H, m), 2.05-2.37 (4H, m), 3.00-3.10 (1H, m), 3.50 (1H, dd, J=14.3, 6.6 Hz), 3.64 (1H, dd, J=14.3, 9.2 Hz), 3.70-3.80 (1H, m), 4.17-4.29 (1H, m), 4.27 (1H, q, J=6.6 Hz), 4.62-4.69 (1H, m), 5.43 (1H, ddd, J=9.9, 9.2, 6.6 Hz), 5.82 (1H, s), 6.62 (1H, d, J=15.8 Hz), 6.87 (1H, dt, J=15.8, 6.6 Hz), 7.14 (1H, d, J=10.3 Hz), 7.31-7.52 (9H, m), 7.53-7.69 (6H, m), 7.69-7.77 (1H, m), 7.85 (1H, d, J=8.4 Hz), 8.12 (1H, d, J=8.4 Hz);

MASS (ES+): m/e 829.28 (M+1).

### EXAMPLE 185

Compound E185 was obtained from the Compound E185 in a manner similar to Example 3.

¹H-NMR (300 MHz, CDCl₃, δ): 0.78 (3H, t, J=7.3 Hz), 1.10 (9H, s), 1.14-1.33 (2H, m), 1.19 (3H, d, J=6.6 Hz), 1.27 (3H, s), 1.38-1.87 (8H, m), 2.06-2.21 (2H, m), 2.23-2.36 (2H, m), 2.52 (2H, t, J=7.7 Hz), 3.01-3.11 (1H, m), 3.50 (1H, dd, J=14.3, 6.6 Hz), 3.64 (1H, dd, J=14.3, 8.8 Hz), 3.70-3.79 (1H, m), 4.14-4.27 (1H, m), 4.19 (1H, q, J=6.6 Hz), 4.64-4.69 (1H, m), 5.38-5.48 (1H, m), 5.82 (1H, s), 7.09 (1H, d, J=11.0 Hz), 7.34-7.53 (9H, m), 7.53-7.78 (7H, m), 7.85 (1H, d, J=8.8 Hz), 8.13 (1H, d, J=8.8 Hz);

MASS (ES+): m/e 831.29 (M+1).

### EXAMPLE 186

Compound E186 was obtained from the Compound E185 in a manner similar to Example 6.

¹H-NMR (300 MHz, CDCl₃, δ): 0.79 (3H, t, J=7.3 Hz), 1.19-1.41 (2H, m), 1.28 (3H, s), 1.39 (3H, d, J=7.0 Hz), 1.55-1.76 (7H, m), 1.76-1.91 (1H, m), 2.04-2.22 (2H, m), 2.23-2.40 (2H, m), 2.41-2.58 (2H, m), 3.05 (1H, dt, J=10.3, 7.3 Hz), 3.50 (1H, dd, J=14.2, 6.6 Hz), 3.56 (1H, d, J=4.4 Hz), 3.64 (1H, dd, J=14.3, 9.2 Hz), 3.69-3.79 (1H, m), 4.15-4.29 (1H, m), 4.23 (1H, q, J=7.0 Hz), 4.63-4.70 (1H, m), 5.43 (1H, ddd, J=10.3, 9.2, 6.6 Hz), 5.86 (1H, s), 7.12 (1H, d, J=10.3 Hz), 7.36-7.40 (2H, m), 7.49 (1H, ddd, J=8.1, 7.0, 1.1 Hz), 7.57 (1H, ddd, J=8.1, 6.6, 1.5 Hz), 7.66 (1H, d, J=10.3 Hz), 7.70-7.77 (1H, m), 7.85 (1H, dd, J=8.1, 1.5 Hz), 8.12 (1H, d, J=8.1 Hz);

MASS (ES+): m/e 593.35 (M+1).

### EXAMPLE 187

Compound E187 was obtained from the Compound (347) in a manner similar to Example 1.

¹H-NMR (300 MHz, CDCl₃, δ): 0.83 (3H, t, J=7.3 Hz), 1.09 (9H, s), 1.22 (3H, d, J=7.0 Hz), 1.28 (3H, s), 1.35-1.92 (14H, m), 2.09-2.39 (4H, m), 2.98 (1H, dd, J=13.2, 5.5 Hz), 3.18-3.42 (4H, m), 3.58-3.77 (2H, m), 3.79-3.93 (1H, m), 4.15-4.27 (1H, m), 4.28 (1H, q, J=7.0 Hz), 4.66 (1H, brd, J=5.9 Hz), 5.12-5.27 (1H, m), 5.91 (1H, s), 6.61 (1H, d, J=15.8 Hz), 6.86 (1H, dt, J=15.8, 6.6 Hz), 7.10 (1H, d, J=10.3 Hz), 7.22-7.49 (11H, m), 7.53-7.73 (4H, m);

MASS (ES+): m/e 890.48 (M+1).

### EXAMPLE 188

Compound E188 was obtained from the Compound E187 in a manner similar to Example 3.

¹H-NMR (300 MHz, CDCl₃, δ): 0.83 (3H, t, J=7.3 Hz), 1.10 (9H, s), 1.15-1.33 (6H, m), 1.19 (3H, d, J=6.6 Hz), 1.29 (3H, s), 1.38-1.92 (10H, m), 2.06-2.41 (4H, m), 2.47-2.56 (2H, m), 2.98 (1H, dd, J=13.2, 5.5 Hz), 3.21-3.40 (4H, m), 3.59-3.77 (2H, m), 3.80-3.92 (1H, m), 4.10-4.26 (2H, m), 4.66 (1H, brd, J=5.9 Hz), 5.19 (1H, dt, J=9.5, 6.2 Hz), 5.91 (1H, s), 7.04 (1H, d, J=10.3 Hz), 7.22-7.49 (10H, m), 7.57-7.70 (5H, m);

MASS (ES+): m/e 892.42 (M+1).

### EXAMPLE 189

Compound E189 was obtained from the Compound E188 in a manner similar to Example 6.

¹H-NMR (300 MHz, CDCl₃, δ): 0.84 (3H, t, J=7.3 Hz), 1.20-1.93 (16H, m), 1.29 (3H, s), 1.38 (3H, d, J=7.3 Hz), 2.06-2.60 (6H, m), 2.98 (1H, dd, J=13.2, 5.9 Hz), 3.19-3.42 (4H, m), 3.56 (1H, brd, J=4.0 Hz), 3.59-3.77 (2H, m), 3.80-3.92 (1H, m), 4.14-4.30 (2H, m), 4.66 (1H, brd, J=6.2 Hz), 5.19 (1H, dt, J=9.9, 6.2 Hz), 5.97 (1H, s), 7.08 (1H, d, J=10.3 Hz), 7.23-7.35 (4H, m), 7.60 (1H, d, J=10.3 Hz);

MASS (ES+): m/e 654.57 (M+1).

### EXAMPLE 190

Compound E190 was obtained from the Compound (350) in a manner similar to Example 1.

¹H-NMR (300 MHz, CDCl₃, δ): 0.84 (3H, t, J=7.3 Hz), 1.09 (9H, s), 1.23 (3H, d, J=6.6 Hz), 1.29 (3H, s), 1.37-1.93 (6H, m), 2.08-2.40 (6H, m), 2.99-3.11 (1H, m), 3.23-3.37 (2H, m), 3.81-3.93 (1H, m), 4.17-4.33 (2H, m), 4.68 (1H, brd, J=6.6 Hz), 5.22 (1H, dt, J=9.5, 6.6 Hz), 5.92 (1H, s), 6.62 (1H, d, J=15.8 Hz), 6.87 (1H, dt, J=15.8, 7.0 Hz), 7.02-7.50 (11H, m), 7.56-7.71 (8H, m), 7.74-7.84 (3H, m);

MASS (ES+): m/e 898.39 (M+1).

### EXAMPLE 191

Compound E191 was obtained from the Compound E190 in a manner similar to Example 3.

¹H-NMR (300 MHz, CDCl₃, δ): 0.84 (3H, t, J=7.3 Hz), 1.04-1.33 (4H, m), 1.10 (9H, s), 1.21 (3H, d, J=6.9 Hz), 1.29 (3H, s), 1.38-1.91 (6H, m), 2.03-2.41 (4H, m), 2.51 (2H, dt, J=7.0, 2.2 Hz), 2.99-3.12 (1H, m), 3.22-3.36 (2H, m), 3.80-3.92 (1H, m), 4.14-4.31 (2H, m), 4.68 (1H, brd, J=5.9 Hz), 5.21 (1H, dt, J=9.5, 7.0 Hz), 5.95 (1H, s), 7.03 (1H, d, J=10.3 Hz), 7.15 (1H, t, J=7.3 Hz), 7.31-7.51 (9H, m), 7.57-7.72 (8H, m), 7.75-7.83 (3H, m);

MASS (ES+): m/e 900.47 (M+1).

### EXAMPLE 192

Compound E192 was obtained from the Compound E191 in a manner similar to Example 6.

¹H-NMR (300 MHz, CDCl₃, δ): 0.84 (3H, t, J=7.3 Hz), 1.16-1.93 (10H, m), 1.29 (3H, s), 1.39 (3H, d, J=7.0 Hz), 2.04-2.59 (6H, m), 3.05 (1H, dd, J=13.6, 6.2 Hz), 3.23-3.37 (2H, m), 3.56 (1H, d, J=4.8 Hz), 3.80-3.89 (1H, m), 4.15-4.30 (2H, m), 4.68 (1H, brd, J=7.0 Hz), 5.21 (1H, dt, J=10.3, 6.6 Hz), 5.96 (1H, s), 7.06 (1H, d, J=10.6 Hz), 7.15 (1H, t, J=7.3 Hz), 7.32-7.43 (4H, m), 7.57-7.68 (3H, m), 7.74-7.85 (3H, m);

MASS (ES+): m/e 662.53 (M+1).

### EXAMPLE 193

Compound E193 was obtained from the Compound (358) in a manner similar to Example 1.

¹H-NMR (300 MHz, CDCl₃, δ): 0.88 (3H, t, J=7.3 Hz), 0.91 (3H, d, J=6.6 Hz), 0.99 (3H, d, J=6.6 Hz), 1.09 (9H, s), 1.22 (3H, d, J=7.0 Hz), 1.31 (3H, s), 1.37-1.71 (4H, m), 1.74-2.02 (3H, m), 2.11-2.44 (6H, m), 3.47-3.60 (1H, m), 3.83-3.96 (1H, m), 4.11-4.29 (1H, m), 4.48 (1H, t, J=10.6 Hz), 4.75 (1H, brd, J=6.3 Hz), 5.83 (1H, s), 6.61 (1H, d, J=15.5 Hz), 6.86 (1H, dt, J=15.5, 7.0 Hz), 7.17 (1H, d, J=10.3 Hz), 7.30-7.47 (7H, m), 7.56-7.69 (4H, m);

MASS (ES+): m/e 731.57 (M+1).

### EXAMPLE 194

Compound E194 was obtained from the Compound E193 in a manner similar to Example 3.

¹H-NMR (300 MHz, CDCl₃, δ): 0.88 (3H, t, J=7.3 Hz), 0.91 (3H, d, J=6.6 Hz), 0.98 (3H, d, J=6.6 Hz), 1.10 (9H, s), 1.15-1.65 (7H, m), 1.18 (3H, d, J=6.6 Hz), 1.30 (3H, s), 1.70-2.01 (4H, m), 2.11-2.44 (4H, m), 2.46-2.54 (2H, m), 3.47-3.59 (1H, m), 3.81-3.96 (1H, m), 4.09-4.24 (2H, m), 4.47 (1H, t, J=10.3 Hz), 4.75 (1H, brd, J=7.3 Hz), 5.83 (1H, s), 7.10 (1H, d, J=10.3 Hz), 7.32-7.48 (7H, m), 7.56-7.67 (4H, m);

MASS (ES+): m/e 733.65 (M+1).

### EXAMPLE 195

Compound E195 was obtained from the Compound E194 in a manner similar to Example 6.

¹H-NMR (300 MHz, CDCl₃, δ): 0.88 (3H, t, J=7.4 Hz), 0.90 (3H, d, J=6.6 Hz), 0.99 (3H, d, J=7.0 Hz), 1.21-1.40 (4H, m), 1.30 (3H, s), 1.38 (3H, d, J=7.0 Hz), 1.52-1.70 (3H, m), 1.70-2.00 (3H, m), 2.12-2.58 (7H, m), 3.47-3.57 (1H, m), 3.56 (1H, d, J=4.4 Hz), 3.83-3.95 (1H, m), 4.13-4.29 (2H, m), 4.48 (1H, t, J=10.3 Hz), 4.75 (1H, dd, J=7.7, 1.8 Hz), 5.85 (1H, s), 7.14 (1H, d, J=10.3 Hz), 7.38 (1H, d, J=9.9 Hz);

MASS (ES+): m/e 495.49 (M+1).

### EXAMPLE 196

Compound E196 was obtained from the Compound (367) in a manner similar to Example 1.

¹H-NMR (300 MHz, CDCl₃, δ): 0.84 (3H, t, J=7.7 Hz), 1.09 (9H, s), 1.22 (3H, d, J=7.0 Hz), 1.29 (3H, s), 1.37-1.93 (6H, m), 2.10-2.38 (6H, m), 2.94 (1H, dd, J=13.6, 6.2 Hz), 3.16-3.34 (2H, m), 3.80-3.92 (1H, m), 3.87 (3H, s), 4.17-4.32 (2H, m), 4.64-4.71 (1H, m), 5.17 (1H, dt, J=9.5, 5.9 Hz), 5.91 (1H, brs), 6.62 (1H, d, J=15.8 Hz), 6.77 (1H, dd, J=8.1, 1.8 Hz), 6.82 (1H, d, J=1.8 Hz), 6.88 (1H, dd, J=15.8, 6.6 Hz), 7.09 (1H, d, J=10.6 Hz), 7.25 (1H, d, J=8.1 Hz), 7.31-7.51 (6H, m), 7.54-7.69 (5H, m);

MASS (ES+): m/e 843.39 (M+1).

### EXAMPLE 197

Compound E197 was obtained from the Compound E196 in a manner similar to Example 3.

¹H-NMR (300 MHz, CDCl₃, δ): 0.84 (3H, t, J=7.3 Hz), 1.11 (9H, s), 1.15-1.35 (10H, m), 1.19 (3H, d, J=6.6 Hz), 1.29 (3H, s), 2.02-2.41 (4H, m), 2.45-2.57 (2H, m), 2.94 (1H, dd, J=13.5, 6.1 Hz), 3.22 (1H, dd, J=13.5, 9.5 Hz), 3.23-3.35 (1H, m), 3.79-3.92 (1H, m), 3.88 (3H, s), 4.10-4.28 (2H, m), 4.68 (1H, brd, J=6.2 Hz), 5.11-5.23 (1H, m), 5.89 (1H, brs), 6.78 (1H, dd, J=8.1, 1.8 Hz), 6.83 (1H, brs), 7.03 (1H, d, J=9.9 Hz), 7.26 (1H, d, J=8.1 Hz), 7.32-7.49 (6H, m), 7.58-7.69 (5H, m);

MASS (ES+): m/e 845.40 (M+1).

### EXAMPLE 198

Compound E198 was obtained from the Compound E197 in a manner similar to Example 6.

¹H-NMR (300 MHz, CDCl₃, δ): 0.84 (3H, t, J=7.3 Hz), 1.18-1.41 (7H, m), 1.29 (3H, s), 1.38 (3H, d, J=7.0 Hz), 1.70-1.92 (3H, m), 2.04-2.59 (6H, m), 2.93 (1H, dd, J=13.6, 6.6 Hz), 3.21 (1H, dd, J=13.6, 9.9 Hz), 3.23-3.34 (1H, m), 3.55 (1H, d, J=4.8 Hz), 3.79-3.92 (1H, m), 3.87 (3H, s), 4.13-4.30 (2H, m), 4.68 (1H, brd, J=5.9 Hz), 5.16 (1H, dt, J=9.5, 5.5 Hz), 5.90 (1H, brs), 6.77 (1H, dd, J=8.1, 1.8 Hz), 6.81 (1H, brs), 7.06 (1H, d, J=10.3 Hz), 7.25 (1H, d, J=8.1 Hz), 7.59 (1H, d, J=10.3 Hz);

MASS (ES+): m/e 607.27 (M+1).

### EXAMPLE 199

Compound E199 was obtained from the Compound (374) in a manner similar to Example 1.

¹H-NMR (300 MHz, CDCl₃, δ): 0.83 (3H, t, J=7.3 Hz), 1.09 (9H, s), 1.23 (3H, d, J=6.6 Hz), 1.29 (3H, s), 1.37-1.92 (6H, m), 2.09-2.41 (6H, m), 2.97 (1H, dd, J=13.6, 6.2 Hz), 3.23 (1H, dd, J=13.6, 9.5 Hz), 3.25-3.36 (1H, m), 3.80-3.94 (1H, m), 4.16-4.32 (2H, m), 4.69 (1H, dd, J=8.1, 2.2 Hz), 5.16 (1H, dt, J=9.5, 6.6 Hz), 5.85 (1H, s), 6.62 (1H, d, J=15.8 Hz), 6.80-6.98 (3H, m), 7.01 (1H, d, J=7.7 Hz), 7.09 (1H, d, J=9.9 Hz), 7.19-7.29 (1H, m), 7.31-7.47 (6H, m), 7.53-7.69 (5H, m);

MASS (ES+): m/e 797.30 (M+1).

### EXAMPLE 200

Compound E200 was obtained from the Compound E199 in a manner similar to Example 3.

¹H-NMR (300 MHz, CDCl₃, δ): 0.83 (3H, t, J=7.3 Hz), 0.99-1.91 (10H, m), 1.10 (9H, s), 1.19 (3H, d, J=7.0 Hz), 1.28 (3H, s), 2.06-2.40 (4H, m), 2.45-2.55 (2H, m), 2.96 (1H, dd, J=13.6, 6.6 Hz), 3.23 (1H, dd, J=13.6, 9.2 Hz), 3.25-3.36 (1H, m), 3.79-3.91 (1H, m), 4.12-4.25 (2H, m), 4.68 (1H, brd, J=8.0 Hz), 5.16 (1H, dt, J=10.3, 6.2 Hz), 5.84 (1H, s), 6.85-7.07 (4H, m), 7.19-7.29 (1H, m), 7.31-7.49 (6H, m), 7.55-7.68 (5H, m);

MASS (ES+): m/e 799.31 (M+1).

### EXAMPLE 201

Compound E201 was obtained from the Compound E200 in a manner similar to Example 6.

¹H-NMR (300 MHz, CDCl₃, δ): 0.83 (3H, t, J=7.3 Hz), 1.20-1.41 (5H, m), 1.29 (3H, s), 1.38 (3H, d, [symbol] =7.0 Hz), 1.52-1.70 (2H, m), 1.71-1.91 (3H, m), 2.08-2.58 (6H, m), 2.97 (1H, dd, J=13.6, 6.2 Hz), 3.22 (1H, dd, J=13.6, 9.2 Hz), 3.26-3.36 (1H, m), 3.56 (1H, d, J=4.8 Hz), 3.81-3.91 (1H, m), 4.15-4.29 (2H, m), 4.69 (1H, dd, J=7.7, 2.2 Hz), 5.16 (1H, dt, J=9.6, 6.6 Hz), 5.86 (1H, s), 6.86-6.98 (2H, m), 7.01 (1H, d, J=7.7 Hz), 7.06 (1H, d, J=10.3 Hz), 7.19-7.30 (1H, m), 7.58 (1H, d, J=10.3 Hz);

MASS (ES+): m/e 561.36 (M+1).

### EXAMPLE 202

Compound E202 was obtained in a manner similar to Example 1.

¹H-NMR (300 MHz, CDCl₃, δ): 0.82 (3H, t, J=7.3 Hz), 1.12 (9H, s), 1.28 (3H, s), 1.31-1.64 (5H, m), 1.68-1.87 (2H, m), 2.07-2.39 (5H, m), 2.96 (1H, dd, J=13.6, 6.6 Hz), 3.18-3.32 (2H, m), 3.80-3.93 (1H, m), 4.12-4.24 (1H, m), 4.66 (1H, brd, J=7.3 Hz), 5.13-5.24 (1H, m), 5.16 (1H, s), 5.78 (1H, s), 6.54 (1H, d, J=15.8 Hz), 6.76 (1H, dt, J=15.8, 6.6 Hz), 7.10 (1H, d, J=10.3 Hz), 7.19-7.57 (19H, m), 7.59-7.66 (2H, m);

MASS (ES+): m/e 841.20 (M+1).

### EXAMPLE 203

Compound E203 was obtained from the Compound E202 in a manner similar to Example 3.

¹H-NMR (300 MHz, CDCl₃, δ): 0.81 (3H, t, J=7.3 Hz), 0.93-1.35 (6H, m), 1.13 (9H, s), 1.27 (3H, s), 1.38-1.54 (1H, m), 1.60-1.86 (3H, m), 2.07-2.47 (6H, m), 2.96 (1H, dd, J=13.6, 6.6 Hz), 3.18-3.33 (2H, m), 3.79-3.90 (1H, m), 4.05-4.16 (1H, m), 4.65 (1H, brd, J=8.1 Hz), 5.09 (1H, s), 5.12-5.23 (1H, m), 5.78 (1H, s), 7.02 (1H, d, J=10.3 Hz), 7.15-7.48 (18H, m), 7.55 (1H, d, J=10.3 Hz), 7.62-7.68 (2H, m);

MASS (ES+): m/e 843.19 (M+1).

### EXAMPLE 204

Compound E204 was obtained from Compound E203 in a manner similar to Example 6.

¹H-NMR (300 MHz, CDCl₃, δ): 0.82 (3H, t, J=7.0 Hz), 1.07-1.90 (10H, m), 1.27 (3H, s), 2.06-2.44 (6H, m), 2.96 (1H, dd, J=13.6, 5.9 Hz), 3.17-3.33 (2H, m), 3.79-3.91 (1H, m), 4.08-4.21 (1H, m), 4.66 (1H, brd, J=7.0 Hz), 5.07 (1H, s), 5.11-5.24 (1H, m), 5.85 (1H, s), 7.08 (1H, d, J=10.3 Hz), 7.16-7.66 (11H, m);

MASS (ES+): m/e 605.36 (M+1).

### EXAMPLE 205

The Compound E138 (147 mg) was reacted with benzyl 2,2,2-trichloroacetimidate (200 mg) in dichloromethane (3 ml) in the presence of the catalytic amount of trifluoromethanol (7.93 mg) under ice-cooling for 1 hour. The temperature of the reaction mixture was raised to ambient temperature and the mixture was stirred for 16 hours. The reaction was quenched with saturated aqueous sodium bicarbonate solution (2 ml) under ice-cooling. The reaction mixture was extracted with ethyl acetate, washed with saturated aqueous sodium bicarbonate solution (20 ml×2) and saturated brine (20 ml), and dried over sodium sulfate. The crude product was purified by reverse phase preparative chromatography and lyophilized from t-butanol to give the objective Compound E205.

¹H-NMR (300 MHz, CDCl₃, δ): 0.83 (3H, t, J=7.7 Hz), 0.88 (3H, d, J=6.6 Hz), 1.21-1.90 (9H, m), 1.28 (3H, s), 1.33 (3H, d, J=7.0 Hz), 2.07-2.43 (3H, m), 2.43-2.71 (3H, m), 2.73 (1H, t, J=8.1 Hz), 2.95 (1H, dd, J=13.6, 5.9 Hz), 3.24 (1H, dd, J=13.6, 9.5 Hz), 3.92 (1H, q, J=7.0 Hz), 4.06 (1H, dd, J=9.5, 7.3 Hz), 4.19 (1H, dt, J=10.3, 7.7 Hz), 4.49 (1H, d, J=11.7 Hz), 4.55 (1H, d, J=11.7 Hz), 4.67 (1H, dd, J=8.1, 2.2 Hz), 5.16 (1H, dt, J=10.3, 5.9 Hz), 5.79 (1H, s), 7.15 (1H, d, J=10.3 Hz), 7.17-7.41 (10H, m), 7.54 (1H, d, J=10.3 Hz);

MASS (ES+): m/e 647.39 (M+1).

### EXAMPLE 206

The Compound E138 (190.7 mg) was reacted with 3,4-dihydro-2H-pyrane (86.4 mg) in dichloromethane (3 ml) in the presence of pyridinium p-toluenesulfonate under ambient temperature for 20 hours. The reaction was quenched with saturated aqueous sodium bicarbonate solution (2 ml). The reaction mixture was extracted with ethyl acetate (50 ml), washed with saturated aqueous sodium bicarbonate solution (20 ml×2) and saturated brine (20 ml), and dried over sodium sulfate. The mixture was purified by preparative thin layer chromatography (eluting with ethyl acetate/hexane=2/1) and lyophilized from t-butanol to give the objective Compound E206.

¹H-NMR (300 MHz, CDCl₃, δ): 0.84 (3H, t, J=7.7 Hz), 0.88 (3H, d, J=6.6 Hz), 1.24-1.95 (15H, m), 1.28 (3H, s), 1.36 (3H, d, J=7.0 Hz), 2.07-2.23 (1H, m), 2.25-2.77 (6H, m), 2.95 (1H, dd, J=13.6, 5.9 Hz), 3.24 (1H, dd, J=13.6, 9.5 Hz), 3.38-3.56 (1H, m), 3.77-3.93 (1H, m), 4.02-4.13 (1H, m), 4.13-4.25 (1H, m), 4.28 (1H, q, J=7.0 Hz), 4.56 (0.5H, dd, J=4.4, 2.9 Hz), 4.61 (0.5H, dd, J=5.1, 2.9 Hz), 4.67 (1H, dd, J=8.1, 1.8 Hz), 5.16 (1H, dt, J=10.3, 6.2 Hz), 5.80 (1H, s), 7.10-7.32 (6H, m), 7.51-7.59 (1H, m);

MASS (ES+): m/e 557.39 (M+1).

### EXAMPLE 207

The Compound E138 (100 mg) was mixed with (2-methoxyethoxy)methyl chloride (44.8 mg) in dichloromethane (2 ml) in the presence of ethyldiisopropylamine (0.156 ml) and the catalytic amount of tetrabutylammonium iodide, and the mixture was refluxed at 100° C. for 36 hours. The reaction mixture was cooled to the ambient temperature and the solvent was removed by evaporation. The residue was extracted with ethyl acetate, and the extract was washed with 1N hydrochloric acid, saturated aqueous sodium bocarbonate and saturated brine and dried over sodium sulfate. The mixture was purified by flush chromatography and lyophilized from ethyl acetate to give the objective Compound E207.

¹H-NMR (300 MHz, CDCl₃, δ): 0.84 (3H, t, J=7.3 Hz), 0.88 (3H, d, J=6.2 Hz), 1.22-1.90 (8H, m), 1.28 (3H, s), 1.32 (3H, d, J=7.0 Hz), 2.08-2.78 (8H, m), 2.95 (1H, dd, J=13.6, 5.9 Hz), 3.24 (1H, dd, J=13.6, 9.9 Hz), 3.39 (3H, s), 3.50-3.60 (2H, m), 3.68-3.76 (2H, m), 4.01-4.25 (3H, m), 4.67 (1H, dd, J=8.4, 2.6 Hz), 4.73 (1H, d, J=7.0 Hz), 4.79 (1H, d, J=7.0 Hz), 5.10-5.22 (1H, m), 5.82 (1H, s), 7.15 (1H, d, J=10.3 Hz), 7.18-7.33 (5H, m), 7.55 (1H, d, J=10.3 Hz);

MASS (ES−): m/e 643.36(M−1).

### EXAMPLE 208

Compound E208 was obtained from the Compound (386) in a manner similar to Example 1.

¹H-NMR (300 MHz, CDCl₃, δ): 0.81 (3H, t, J=7.5 Hz), 1.09 (3×3H, s), 1.22 (3H, d, J=6.5 Hz), 1.29 (3H, s), 1.38-1.52 (2H, m), 1.56-1.93 (4H, m), 2.02-2.42 (6H, m), 3.01 (1H, dd, J=13.5, 6.5 Hz), 3.22 (1H, dd, J=13.5, 9 Hz), 3.34 (1H, m), 3.86 (1H, m), 4.23 (1H, m), 4.27 (1H, q, J=6.5 Hz), 4.68 (1H, brd, J=8 Hz), 5.22 (1H, m), 5.83 (1H, s), 6.63 (1H, d, J=15.5 Hz), 6.87 (1H, dt, J=15.5, 7 Hz), 7.02 (1H, d, J=10 Hz), 7.17 (2×1H, brd, J=5.5 Hz), 7.31-7.49 (6H, m), 7.56-7.72 (5H, m), 8.51 (2×1H, brd, J=5.5 Hz);

MASS (ES+): m/e 780.29.

### EXAMPLE 209

Compound E209 was obtained from the Compound E208 in a manner similar to Example 3.

¹H-NMR (300 MHz, CDCl₃, δ): 0.81 (3H, t, J=7.5 Hz), 1.10 (3×3H, s), 1.16-1.34 (4H, m), 1.18 (3H, d, J=6.5 Hz), 1.28 (3H, s), 1.38-1.68 (3H, m), 1.72-1.92 (3H, m), 2.02-2.40 (4H, m), 2.51 (2H, m), 3.01 (1H, dd, J=13.5, 6.5 Hz), 3.21 (1H, dd, J=13.5, 9 Hz), 3.34 (1H, m), 3.85 (1H, m), 4.13-4.26 (2H, m), 4.68 (1H, brd, J=8 Hz), 5.21 (1H, m), 5.84 (1H, s), 6.96 (1H, d, J=10 Hz), 7.17 (2×1H, d, J=6 Hz), 7.32-7.49 (6H, m), 7.57-7.72 (5H, m), 8.51 (2×1H, d, J=6 Hz);

MASS (ES+): m/e 782.38.

### EXAMPLE 210

Compound E210 was obtained from the Compound E209 in a manner similar to Example 6.

¹H-NMR (300 MHz, CDCl₃, δ): 0.82 (3H, t, J=7.3 Hz), 1.20-1.45 (4H, m), 1.29 (3H, s), 1.38 (3H, d, J=7 Hz), 1.54-1.92 (6H, m), 2.06-2.56 (6H, m), 3.01 (1H, dd, J=13.5, 7 Hz), 3.21 (1H, dd, J=13.5, 8.5 Hz), 3.34 (1H, m), 3.56 (1H, br), 3.86 (1H, m), 4.15-4.30 (2H, m), 4.69 (1H, brd, J=8 Hz), 5.21 (1H, ddd, J=10.5, 8.5, 7 Hz), 5.85 (1H, s), 6.99 (1H, d, J=10 Hz), 7.17 (2×1H, d, J=6 Hz), 7.63 (1H, d, J=10.5 Hz), 8.51 (2×1H, d, J=6 Hz);

MASS (ES+): m/e 543.38;

[α]_{D}²²=−113.7° (c=0.20, CHCl₃).

### EXAMPLE 211

Compound E211 was obtained from the Compound (390) in a manner similar to Example 1.

¹H-NMR (300 MHz, CDCl₃, δ): 0.84 (3H, t, J=7.3 Hz), 1.09 (3×3H, s), 1.22 (3H, d, J=7 Hz), 1.38-1.51 (3H, m), 1.56-1.91 (4H, m), 2.08-2.40 (6H, m), 2.89 (1H, dd, J=13.5, 6 Hz), 3.18 (1H, dd, J=13.5, 9.5 Hz), 3.26 (1H, m), 3.86 (1H, m), 4.21 (1H, dt, J=10.5, 7.7 Hz), 4.27 (1H, q, J=7 Hz), 4.50 (1H, ddd, J=5, 1.5, 1.5 Hz), 4.66 (1H, dd, J=8, 2 Hz), 5.13 (1H, ddd, J=10, 9.5, 6 Hz), 5.27 (1H, ddt, J=10.5, 1.5, 1.5 Hz), 5.40 (1H, ddt, J=17.3, 1.5, 1.5 Hz), 5.79 (1H, s), 6.04 (1H, ddt, J=17.3, 10.5, 5 Hz), 6.62 (1H, brd, J=15, 7 Hz), 6.82 (2×1H, d, J=8.5 Hz), 6.86 (1H, dt, J=15.7, 7 Hz), 7.13 (1H, d, J=10.5 Hz), 7.13 (2×1H, d, J=8.5 Hz), 7.30-7.48 (6H, m), 7.50 (1H, d, J=10 Hz), 7.56-7.69 (4H, m);

MASS (ES−): m/e 836.08.

### EXAMPLE 212

Compound E212 was obtained from the Compound E211 in a manner similar to Example 3.

¹H-NMR (300 MHz, CDCl₃, δ): 0.84 (3H, t, J=7.3 Hz), 1.02 (3H, t, J=7.4 Hz), 1.10 (3×3H, s), 1.18 (3H, d, J=7 Hz), 1.21-1.32 (4H, m), 1.28 (3H, s), 1.38-1.64 (3H, m), 1.68-1.85 (5H, m), 2.07-2.40 (4H, m), 2.51 (2H, m), 2.88 (1H, dd, J=13.5, 6 Hz), 3.18 (1H, dd, J=13.5, 10 Hz), 3.26 (1H, m), 3.85 (1H, m), 3.88 (2H, t, J=6.6 Hz), 4.13-4.23 (2H, m), 4.66 (1H, dd, J=8, 2.5 Hz), 5.13 (1H, ddd, J=10.2, 10, 6 Hz), 5.79 (1H, s), 6.80 (2×1H, d, J=8.5 Hz), 7.08 (1H, d, J=10.3 Hz), 7.12 (2×1H, d, J=8.5 Hz), 7.33-7.48 (6H, m), 7.54 (1H, d, J=10.2 Hz), 7.58-7.67 (4H, m);

MASS (ES+): m/e 839.32.

### EXAMPLE 213

Compound E213 was obtained from the Compound E212 in a manner similar to Example 6.

¹H-NMR (300 MHz, CDCl₃, δ): 0.84 (3H, t, J=7.3 Hz), 1.02 (3H, t, J=7.4 Hz), 1.22-1.40 (4H, m), 1.28 (3H, s), 1.38 (3H, d, J=7.3 Hz), 1.52-1.70 (3H, m), 1.71-1.90 (5H, m), 2.06-2.58 (6H, m), 2.88 (1H, dd, J=13.5, 6 Hz), 3.17 (1H, dd, J=13.5, 10 Hz), 3.26 (1H, m), 3.55 (1H, d, J=4.7 Hz), 3.86 (1H, m), 3.87 (2H, t, J=6.6 Hz), 4.13-4.29 (2H, m), 4.66 (1H, m), 5.13 (1H, ddd, J=10, 10, 6 Hz), 5.81 (1H, s), 6.80 (2×1H, d, J=8.5 Hz), 7.11 (1H, d, J=10 Hz), 7.12 (2×1H, d, J=8.5 Hz), 7.52 (1H, d, J=10 Hz);

MASS (ES+): m/e 601.44

[α]_{D}²²=−121.0° (c=0.23, CHCl₃).

### EXAMPLE 214

Compound E214 was obtained from the Compound (393) in a manner similar to Example 1.

¹H-NMR (300 MHz, CDCl₃, δ): 0.84 (3H, t, J=7.3 Hz), 1.22 (3H, d, J=7 Hz), 1.29 (3H, s), 1.31 (2×3H, d, J=6 Hz), 1.38-1.53 (2H, m), 1.54-1.89 (4H, m), 2.08-2.39 (6H, m), 2.88 (1H, dd, J=13.5, 6 Hz), 3.17 (1H, dd, J=13.5, 9.5 Hz), 3.26 (1H, m), 3.86 (1H, m), 4.21 (1H, dt, J=10, 7.5 Hz), 4.27 (1H, q, J=7 Hz), 4.49 (1H, qq, J=6, 6 Hz), 4.67 (1H, dd, J=8, 2 Hz), 5.14 (1H, ddd, J=10, 9.5, 6 Hz), 5.80 (1H, s), 6.61 (1H, d, J=16 Hz), 6.79 (2×1H, d, J=8.5 Hz), 6.86 (1H, dt, J=16, 7 Hz), 7.12 (2×1H, d, J=8.5 Hz), 7.14 (1H, d, J=10 Hz), 7.30-7.47 (6H, m), 7.50 (1H, d, J=10 Hz), 7.55-7.68 (4H, m);

MASS (ES+): m/e 837.53.

### EXAMPLE 215

Compound E215 was obtained from the Compound E214 in a manner similar to Example 3.

¹H-NMR (300 MHz, CDCl₃, δ): 0.84 (3H, t, J=7.3 Hz), 1.10 (3×3H, s), 1.18 (3H, d, J=7 Hz), 1.20-1.33 (4H, m), 1.28 (3H, s), 1.31 (2×3H, d, J=6 Hz), 1.38-1.51 (2H, m), 1.55-1.62 (1H, m), 1.70-1.87 (3H, m), 2.08-2.24 (2H, m), 2.25-2.39 (2H, m), 2.51 (2H, m), 2.88 (1H, dd, J=13.5, 6 Hz), 3.17 (1H, dd, J=13.5, 10 Hz), 3.26 (1H, m), 3.85 (1H, m), 4.18 (1H, m), 4.18 (1H, q, J=7 Hz), 4.49 (1H, qq, J=6, 6 Hz), 4.66 (1H, dd, J=8, 2.5 Hz), 5.13 (1H, ddd, J=10, 10, 6 Hz), 5.80 (1H, s), 6.89 (2×1H, d, J=8.8 Hz), 7.08 (1H, d, J=10 Hz), 7.12 (2×1H, d, J=8.8 Hz), 7.33-7.48 (6H, m), 7.54 (1H, d, J=10 Hz), 7.58-7.68 (4H, m);

MASS (ES+): m/e 839.58.

### EXAMPLE 216

Compound E216 was obtained from the Compound E215 in a manner similar to Example 6.

¹H-NMR (300 MHz, CDCl₃, δ): 0.84 (3H, t, J=7.3 Hz), 1.22-1.40 (4H, m), 1.28 (3H, s), 1.31 (2×3H, d, J=6 Hz), 1.38 (3H, d, J=7 Hz), 1.54-1.90 (6H, m), 2.08-2.58 (6H, m), 2.88 (1H, dd, J=13.5, 6 Hz), 3.17 (1H, dd, J=13.5, 9.5 Hz), 3.26 (1H, m), 3.56 (1H, d, J=4.5 Hz), 3.86 (1H, m), 4.14-4.29 (2H, m), 4.49 (1H, qq, J=6, 6 Hz), 4.67 (1H, dd, J=8, 2 Hz), 5.13 (1H, ddd, J=10, 9.5, 6 Hz), 5.81 (1H, s), 6.79 (2×1H, d, J=8.7 Hz), 7.12 (1H, d, J=10 Hz), 7.12 (2×1H, d, J=8.7 Hz), 7.52 (1H, d, J=10 Hz);

MASS (ES+): m/e 601.39;

[α]_{D}²³=−121.4° (c=0.25, CHCl₃).

### EXAMPLE 217

Compound E217 was obtained from the Compound (397) in a manner similar to Example 1.

¹H-NMR (300 MHz, CDCl₃, δ): 0.84 (3H, t, J=7.3 Hz), 0.96 (3H, t, J=7.3 Hz), 1.09 (3×3H, s), 1.22 (3H, d, J=6.7 Hz), 1.28 (3H, s), 1.38-1.91 (10H, m), 2.08-2.40 (6H, m), 2.88 (1H, dd, J=13.5, 6 Hz), 3.18 (1H, dd, J=13.5, 10 Hz), 3.86 (1H, m), 3.92 (2H, t, J=6.5 Hz), 4.21 (1H, dt, J=10, 7.7 Hz), 4.27 (1H, q, J=6.7 Hz), 4.66 (1H, dd, J=8, 2.5 Hz), 5.13 (1H, ddd, J=10, 10, 6 Hz), 5.79 (1H, s), 6.61 (1H, d, J=15.8 Hz), 6.80 (2×1H, d, J=8.5 Hz), 6.86 (1H, dt, J=15.8, 7 Hz), 7.12 (2×1H, d, J=8.5 Hz), 7.31-7.47 (6H, m), 7.50 (1H, d, J=10 Hz), 7.56-7.69 (4H, m);

MASS (ES+): m/e 851.37.

### EXAMPLE 218

Compound E218 was obtained from the Compound E217 in a manner similar to Example 3.

¹H-NMR (300 MHz, CDCl₃, δ): 0.84 (3H, t, J=7.3 Hz), 0.96 (3H, t, J=7.3 Hz), 1.10 (3×3H, s), 1.18 (3H, d, J=6.5 Hz), 1.20-1.32 (4H, m), 1.28 (3H, s), 1.39-1.62 (6H, m), 1.68-1.87 (4H, m), 2.08-2.40 (6H, m), 2.51 (2H, m), 2.88 (1H, dd, J=13.5, 6 Hz), 3.18 (1H, dd, J=13.5, 10 Hz), 3.26 (1H, m), 3.85 (1H, m), 3.92 (2H, t, J=6.5 Hz), 4.10-4.23 (2H, m), 4.66 (1H, dd, J=8, 2 Hz), 5.13 (1H, ddd, J=10, 10, 6 Hz), 5.80 (1H, s), 6.80 (2×1H, d, J=8.5 Hz), 7.08 (1H, d, J=10.5 Hz), 7.13 (2×1H, d, J=8.5 Hz), 7.33-7.48 (6H, m), 7.54 (1H, d, J=10 Hz), 7.58-7.68 (4H, m);

MASS (ES+): m/e 853.43.

### EXAMPLE 219

Compound E219 was obtained from the Compound E218 in a manner similar to Example 6.

¹H-NMR (300 MHz, CDCl₃, δ): 0.84 (3H, t, J=7.3 Hz), 0.96 (3H, t, J=7.3 Hz), 1.21-1.89 (14H, m), 1.28 (3H, s), 1.38 (3H, d, J=7.3 Hz), 2.07-2.57 (6H, m), 2.88 (1H, dd, J=13.5, 6 Hz), 3.17 (1H, dd, J=13.5, 10 Hz), 3.26 (1H, m), 3.55 (1H, d, J=5 Hz), 3.86 (1H, m), 3.92 (2H, t, J=6.5 Hz), 4.13-4.29 (2H, m), 4.67 (1H, dd, J=8, 2 Hz), 5.13 (1H, ddd, J=10, 10, 6 Hz), 5.84 (1H, s), 6.80 (2×1H, d, J=8.3 Hz), 7.12 (2×1H, d, J=8.3 Hz), 7.12 (1H, d, J=10 Hz), 7.52 (1H, d, J=10 Hz);

MASS (ES+): m/e 615.44.

### EXAMPLE 220

Compound E220 was obtained from the Compound (406) in a manner similar to Example 1.

¹H-NMR (300 MHz, CDCl₃, δ): 0.95 (3H, t, J=7.4 Hz), 1.09 (3×3H, s), 1.23 (3H, d, J=6.5 Hz), 1.39-1.53 (2H, m), 1.58-1.90 (6H, m), 1.74 (3H, s), 2.10-2.38 (4H, m), 2.95 (1H, dd, J=13.5, 6 Hz), 3.20 (1H, m), 3.27 (1H, dd, J=13.5, 10 Hz), 3.88 (1H, m), 4.22 (1H, m), 4.27 (1H, q, J=6.5 Hz), 4.67 (1H, dd, J=8, 2 Hz), 5.16 (1H, ddd, J=10, 10, 6 Hz), 5.85 (1H, s), 6.61 (1H, d, J=16 Hz), 6.87 (1H, dt, J=16, 7 Hz), 7.15 (1H, d, J=10 Hz), 7.18-7.49 (12H, m), 7.56-7.69 (4H, m);

MASS (ES+): m/e 779.37.

### EXAMPLE 221

Compound E221 was obtained from the Compound E220 in a manner similar to Example 3.

¹H-NMR (300 MHz, CDCl₃, δ): 0.95 (3H, t, J=7.3 Hz), 1.10 (3×3H, s), 1.19 (3H, d, J=7 Hz), 1.21-1.32 (4H, m), 1.40-1.52 (2H, m), 1.54-1.86 (6H, m), 1.73 (3H, s), 2.17 (1H, m), 2.31 (1H, m), 2.51 (2H, m), 2.95 (1H, dd, J=13.5, 5.5 Hz), 3.20 (1H, m), 3.28 (1H, dd, J=13.5, 10 Hz), 3.87 (1H, m), 4.12-4.24 (2H, m), 4.65 (1H, dd, J=8, 2 Hz), 5.16 (1H, ddd, J=10, 10, 5.5 Hz), 5.86 (1H, s), 7.09 (1H, d, J=10 Hz), 7.17-7.32 (5H, m), 7.33-7.51 (7H, m), 7.58-7.68 (4H, m);

MASS (ES+): m/e 803.38.

### EXAMPLE 222

Compound E222 was obtained from the Compound E221 in a manner similar to Example 6.

¹H-NMR (300 MHz, CDCl₃, δ): 0.95 (3H, t, J=7.4 Hz), 1.24-1.41 (4H, m), 1.38 (3H, d, J=7 Hz), 1.58-1.88 (8H, m), 1.73 (3H, s), 2.15 (1H, m), 2.27-2.58 (3H, m), 2.95 (1H, dd, J=13.5, 5.5 Hz), 3.20 (1H, m), 3.27 (1H, dd, J=13.5, 10 Hz), 3.55 (1H, d, J=4.7 Hz), 3.86 (1H, m), 4.20 (1H, dt, J=10, 7.5 Hz), 4.22 (1H, q, J=7 Hz), 4.65 (1H, dd, J=8, 2 Hz), 5.15 (1H, ddd, J=10, 10, 5.5 Hz), 5.86 (1H, s), 7.12 (1H, d, J=10 Hz), 7.17-7.32 (5H, m), 7.44 (1H, d, J=10 Hz);

MASS (ES+): m/e 543.38;

[α]_{D}²³=−106.8° (c=0.23, CHCl₃).

### EXAMPLE 223

Compound E223 was obtained from the Compound (409) in a manner similar to Example 1.

¹H-NMR (300 MHz, CDCl₃, δ): 0.84 (3H, t, J=7.3 Hz), 1.09 (3×3H, s), 1.22 (3H, d, J=6.6 Hz), 1.28 (3H, s), 1.38-1.51 (2H, m), 1.53-1.94 (12H, m), 2.08-2.39 (6H, m), 2.88 (1H, dd, J=13.5, 5.8 Hz), 3.17 (1H, dd, J=13.5, 9.9 Hz), 3.26 (1H, m), 3.86 (1H, m), 4.21 (1H, dt, J=10.1, 7.7 Hz), 4.27 (1H, q, J=6.6 Hz), 4.63-4.74 (2H, m), 5.13 (1H, ddd, J=10.2, 9.9, 5.8 Hz), 5.83 (1H, s), 6.61 (1H, d, J=15.6 Hz), 6.78 (2×1H, d, J=8.8 Hz), 6.86 (1H, dt, J=15.6, 6.8 Hz), 7.11 (2×1H, d, J=8.8 Hz), 7.13 (1H, d, J=10.1 Hz), 7.31-7.48 (6H, m), 7.50 (1H, d, J=10.2 Hz), 7.56-7.69 (4H, m);

MASS (ES+): m/e 863.22.

### EXAMPLE 224

Compound E224 was obtained from the Compound E223 in a manner similar to Example 3.

¹H-NMR (300 MHz, CDCl₃, δ): 0.84 (3H, t, J=7.3 Hz), 1.10 (3×3H, s), 1.15-1.32 (4H, m), 1.18 (3H, d, J=6.7 Hz), 1.28 (3H, s), 1.39-1.67 (6H, m), 1.68-1.95 (8H, m), 2.08-2.40 (4H, m), 2.51 (2H, m), 2.88 (1 Hz dd, J=13.5, 6 Hz), 3.17 (1H, dd, J=13.5, 10 Hz), 3.26 (1H, m), 3.85 (1H, m), 4.13-4.24 (2H, m), 4.63-4.74 (2H, m), 5.13 (1H, ddd, J=10, 10, 6 Hz), 5.82 (1H, s), 6.77 (2×1H, d, J=8.5 Hz), 7.08 (1H, d, J=10 Hz), 7.11 (2×1H, d, J=8.5 Hz), 7.32-7.48 (6H, m), 7.54 (1H, d, J=10 Hz), 7.58-7.69 (4H, m);

MASS (ES+): m/e 865.88.

### EXAMPLE 225

Compound E225 was obtained from the Compound E224 in a manner similar to Example 6.

¹H-NMR (300 MHz, CDCl₃, δ): 0.84 (3H, t, J=7.3 Hz), 1.22-1.40 (4H, m), 1.28 (3H, s), 1.38 (3H, d, J=7 Hz), 1.52-1.70 (6H, m), 1.71-1.95 (8H, m), 2.08-2.57 (6H, m), 2.87 (1H, dd, J=13.5, 6 Hz), 3.17 (1H, dd, J=13.5, 10 Hz), 3.26 (1H, m), 3.55 (1H, d, J=4.5 Hz), 3.86 (1H, m), 4.13-4.29 (2H, m), 4.63-4.74 (2H, m), 5.13 (1H, ddd, J=10, 10, 6 Hz), 5.81 (1H, s), 6.77 (2×1H, d, J=8.7 Hz), 7.11 (2×1H, d, J=8.7 Hz), 7.12 (1H, d, J=10 Hz), 7.51 (1H, d, J=10 Hz);

MASS (ES+): m/e 627.10.

### EXAMPLE 226

Compound E226 was obtained from the Compound (412) in a manner similar to Example 1.

¹H-NMR (300 MHz, CDCl₃, δ): 0.84 (3H, t, J=7.3 Hz), 1.10 (3×3H, s), 1.22 (3H, d, J=6.5 Hz), 1.28 (3H, s), 1.38-1.52 (2H, m), 1.56-1.92 (4H, m), 2.08-2.39 (6H, m), 2.90 (1H, dd, J=14, 6 Hz), 3.18 (1H, dd, J=14, 9.5 Hz), 3.26 (1H, m), 3.80 (3H, s), 3.86 (1H, m), 4.15-4.31 (2H, m), 4.60 (2H, s), 4.67 (1H, dd, J=8, 2.5 Hz), 5.13 (1H, ddd, J=10, 9.5, 6 Hz), 5.83 (1H, s), 6.62 (1H, d, J=8.5 Hz), 6.85 (1H, m), 7.12 (1H, d, J=10 Hz), 7.15 (2×1H, d, J=8.5 Hz), 7.31-7.49 (6H, m), 7.52 (1H, d, J=10 Hz), 7.57-7.69 (4H, m);

MASS (ES+): m/e 867.27.

### EXAMPLE 227

Compound E227 was obtained from the Compound E226 in a manner similar to Example 3.

¹H-NMR (300 MHz, CDCl₃, δ): 0.83 (3H, t, J=7.3 Hz), 1.10 (3×3H, s), 1.16-1.32 (4H, m), 1.18 (3H, d, J=6.6 Hz), 1.28 (3H, s), 1.40-1.51 (2H, m), 1.59 (1H, m), 1.69-1.87 (3H, m), 2.08-2.39 (4H, m), 2.51 (2H, m), 2.89 (1H, dd, J=13.5, 6 Hz), 3.18 (1H, dd, J=13.5, 10 Hz), 3.26 (1H, m), 3.80 (3H, s), 3.85 (1H, m), 4.18 (1H, m), 4.25 (1H, q, J=6.6 Hz), 4.60 (2H, s), 4.67 (1H, m), 5.13 (1H, ddd, J=10, 10, 6 Hz), 5.83 (1H, s), 6.81 (2×1H, d, J=8.8 Hz), 7.06 (1H, d, J=10 Hz), 7.15 (2×1H, d, J=10 Hz), 7.33-7.49 (6H, m), 7.55 (1H, d, J=10 Hz), 7.59-7.70 (4H, m);

MASS (ES+): m/e 869.20.

### EXAMPLE 228

Compound E228 was obtained from the Compound E227 in a manner similar to Example 6.

¹H-NMR (300 MHz, CDCl₃, δ): 0.84 (3H, t, J=7.3 Hz), 1.22-1.44 (4H, m), 1.28 (3H, s), 1.38 (3H, d, J=7 Hz), 1.54-1.70 (3H, m), 1.72-1.90 (3H, m), 2.08-2.58 (6H, m), 2.89 (1H, dd, J=13.5, 6 Hz), 3.18 (1H, dd, J=13.5, 9.5 Hz), 3.26 (1H, m), 3.57 (1H, d, J=4.5 Hz), 3.80 (3H, s), 3.85 (1H, m), 4.14-4.29 (2H, m), 4.60 (2H, s), 4.67 (1H, m), 5.13 (1H, ddd, J=10, 9.5, 6 Hz), 5.86 (1H, s), 6.82 (2×1H, d, J=8.5 Hz), 7.10 (1H, d, J=10 Hz), 7.15 (2×1H, d, J=8.5 Hz), 7.53 (1H, d, J=10 Hz);

MASS (ES+): m/e 631.39.

### EXAMPLE 229

The Compound E227 (155 mg) was hydrolyzed with 1N aqueous sodium hydroxide (0.357 ml) in methanol (4 ml) under ambient temperature for 1 hour. The reaction mixture was neutralized with 1N hydrochloric acid and the solvent was removed by evaporation. The residue was partitioned between ethyl acetate and saturated brine, and the ethyl acetate layer was dried over sodium sulfate and evaporated. The residue was purified by thin layer chromatography (eluting with methanol/CHCl₃=1/5) to give the objective Compound E229 as a white foam.

¹H-NMR (300 MHz, CDCl₃, δ): 0.82 (3H, t, J=7.4 Hz), 1.10 (3×3H, s), 1.14-1.32 (4H, m), 1.18 (3H, d, J=7 Hz), 1.27 (3H, s), 1.36-1.86 (6H, m), 2.02-2.56 (6H, m), 2.84 (1H, dd, J=13.5, 5.5 Hz), 3.08-3.28 (2H, m), 3.81 (1H, m), 4.17 (1H, m), 4.18 (1H, q, J=7 Hz), 4.54 (2H, s), 4.63 (1H, m), 5.10 (1H, m), 5.95 (1H, s), 6.80 (2×1H, d, J=8.5 Hz), 7.10 (2×1H, d, J=8.5 Hz), 7.15 (1H, d, J=10 Hz), 7.32-7.47 (6H, m), 7.55-7.67 (5H, m);

MASS (ES−): m/e 853.39.

### EXAMPLE 230

Compound E230 was obtained from the Compound E229 in a manner similar to Example 6.

¹H-NMR (300 MHz, CDCl₃, δ): 0.84 (3H, t, J=7.3 Hz), 1.22-1.40 (4H, m), 1.28 (3H, s), 1.38 (3H, d, J=7 Hz), 1.54-1.88 (6H, m), 2.06-2.57 (6H, m), 2.89 (1H, dd, J=13.5, 6 Hz), 3.18 (1H, dd, J=13.5, 9.5 Hz), 3.26 (1H, m), 3.84 (1H, m), 4.19 (1H, m), 4.24 (1H, q, J=7 Hz), 4.60 (1H, s), 4.67 (1H, m), 5.12 (1H, ddd, J=10, 9.5, 6 Hz), 5.97 (1H, s), 6.84 (2×1H, d, J=8.5 Hz), 7.12 (1H, d, J=10 Hz), 7.15 (2×1H, d, J=8.5 Hz), 7.56 (1H, d, J=10 Hz);

MASS (ES−): m/e 615.46.

### EXAMPLE 231

Compound E231 was obtained from the Compound (415) in a manner similar to Example 1.

¹H-NMR (300 MHz, CDCl₃, δ): 0.84 (3H, t, J=7.3 Hz), 1.09 (3×3H, s), 1.22 (3H, d, J=7 Hz), 1.29 (3H, s), 1.38-1.51 (2H, m), 1.55-1.91 (4H, m), 2.08-2.39 (6H, m), 2.95 (1H, dd, J=13.5, 6 Hz), 3.23 (1H, dd, J=13.5, 10 Hz), 3.28 (1H, m), 3.87 (1H, m), 4.21 (1H, dt, J=10.2, 7.7 Hz), 4.27 (1H, q, J=7 Hz), 4.67 (1H, m), 5.18 (1H, ddd, J=10, 10, 6 Hz), 5.21 (1H, dd, J=11.8, 1 Hz), 5.71 (1H, dd, J=17.6, 1 Hz), 5.88 (1H, s), 6.62 (1H, d, J=15.8 Hz), 6.67 (1H, dd, J=17.6, 11.8 Hz), 6.87 (1H, dt, J=15.8, 7 Hz), 7.13 (1H, d, J=10.2 Hz), 7.19 (2×1H, d, J=8 Hz), 7.30-7.48 (8H, m), 7.55 (1H, d, J=10 Hz), 7.57-7.68 (4H, m);

MASS (ES+): m/e 805.62.

### EXAMPLE 232

Compound E232 was obtained from the Compound E231 in a manner similar to Example 3.

¹H-NMR (300 MHz, CDCl₃, δ): 0.83 (3H, t, J=7.5 Hz), 1.10 (3×3H, s), 1.15-1.32 (4H, m), 1.18 (3H, d, J=7 Hz), 1.20 (3H, t, J=7.5 Hz), 1.28 (3H, s), 1.38-1.64 (3H, m), 1.70-1.87 (3H, m), 2.08-2.39 (4H, m), 2.51 (2H, m), 2.60 (2H, q, J=7.5 Hz), 2.92 (1H, dd, J=13.5, 6 Hz), 3.21 (−1H, dd, J=13.5, 9.5 Hz), 3.28 (1H, m), 3.86 (1H, m), 4.18 (1H, m), 4.18 (1H, q, J=7 Hz), 4.68 (1H, m), 5.17 (1H, ddd, J=10, 9.5, 6 Hz), 5.88 (1H, s), 7.09 (1H, d, J=10 Hz), 7.10 (2×1H, d, J=8.5 Hz), 7.14 (2×1H, d, J=8.5 Hz), 7.33-7.48 (6H, m), 7.56 (1H, d, J=10 Hz), 7.58-7.68 (4H, m);

MASS (ES+): m/e 809.60.

### EXAMPLE 233

Compound E233 was obtained from the Compound E232 in a manner similar to Example 6.

¹H-NMR (300 MHz, CDCl₃, δ): 0.83 (3H, t, J=7.3 Hz), 1.20 (3H, t, J=7.7 Hz), 1.24-1.40 (4H, m), 1.28 (3H, s), 1.38 (3H, d, J=7 Hz), 1.52-1.90 (6H, m), 2.08-2.55 (6H, m), 2.60 (2H, t, J=7.7 Hz), 2.93 (1H, dd, J=13.5, 6 Hz), 3.20 (1H, dd, J=13.5, 9.5 Hz), 3.28 (1H, m), 3.56 (1H, d, J=4.5 Hz), 3.87 (1H, m), 4.14-4.28 (2H, m), 4.68 (1H, dd, J=8, 2 Hz), 5.17 (1H, ddd, J=10, 9.5, 6 Hz), 5.90 (1H, s), 7.10 (2×1H, d, J=8.5 Hz), 7.12-7.17 (3H, m), 7.54 (1H, d, J=10 Hz);

MASS (ES+): m/e 571.58;

[α]_{D}²⁵=−119.3° (c=0.24, CHCl₃).

### EXAMPLE 234

Compound E234 was obtained from the Compound (418) in a manner similar to Example 1.

¹H-NMR (300 MHz, CDCl₃, δ): 0.83 (3H, t, J=7 Hz), 1.09 (3×3H, s), 1.23 (3H, d, J=7 Hz), 1.28 (3H, s), 1.36-1.88 (6H, m), 2.08-2.38 (6H, m), 2.89 (1H, dd, J=13.5, 6 Hz), 3.18 (1H, dd, J=13.5, 9.5 Hz), 3.26 (1H, m), 4.21 (1H, m), 4.27 (1H, q, J=7 Hz), 4.67 (1H, m), 5.13 (1H, m), 5.17 (2H, s), 5.88 (1H, s), 6.62 (1H, brd, J=16 Hz), 6.87 (1H, dt, J=16, 7 Hz), 6.90 (2×1H, d, J=8.7 Hz), 7.14 (1H, d, J=10 Hz), 7.14 (2×1H, d, J=8.7 Hz), 7.23 (1H, m), 7.30-7.75 (13H, m), 8.59 (1H, m);

MASS (ES+): m/e 886.46.

### EXAMPLE 235

Compound E235 was obtained from the Compound E234 in a manner similar to Example 3.

¹H-NMR (300 MHz, CDCl₃, δ): 0.83 (3H, t, J=7.3 Hz), 1.10 (3×3H, s), 1.16-1.32 (4H, m), 1.18 (3H, d, J=7 Hz), 1.28 (3H, s), 1.38-1.88 (6H, m), 2.07-2.40 (4H, m), 2.51 (2H, m), 2.89 (1H, dd, J=13.5, 6 Hz), 3.18 (1H, dd, J=13.5, 9.5 Hz), 3.26 (1H, m), 3.85 (1H, m), 4.12-4.24 (2H, m), 4.67 (1H, m), 5.13 (1H, ddd, J=10, 9.5, 6 Hz), 5.17 (2H, s), 5.83 (1H, s), 6.90 (2×1H, d, J=8.5 Hz), 7.08 (1H, d, J=10 Hz), 7.15 (2×1H, d, J=8.5 Hz), 7.22 (1H, dd, J=7.5, 5 Hz), 7.33-7.48 (6H, m), 7.50 (1H, d, J=7.5 Hz), 7.55 (1H, d, J=10 Hz), 7.59-7.67 (4H, m), 7.70 (1H, ddd, J=7.5, 7.5, 1.5 Hz), 8.59 (1H, brd, J=5 Hz);

MASS (ES+): m/e 888.43.

### EXAMPLE 236

Compound E236 was obtained from the Compound E235 in a manner similar to Example 6.

¹H-NMR (300 MHz, CDCl₃, δ): 0.83 (3H, t, J=7.3 Hz), 1.20-1.42 (4H, m), 1.28 (3H, s), 1.38 (3H, d, J=7 Hz), 1.52-1.90 (6H, m), 2.06-2.58 (6H, m), 2.89 (1H, dd, J=13.5, 6 Hz), 3.18 (1H, dd, J=13.5, 9.5 Hz), 3.26 (1H, m), 3.57 (1H, br), 3.85 (1H, m), 4.13-4.29 (2H, m), 4.67 (1H, m), 5.13 (1H, m), 5.17 (2H, s), 5.93 (1H, s), 6.90 (2×1H, d, J=8.6 Hz), 7.12 (1H, d, J=10 Hz), 7.14 (2×1H, d, J=8.6 Hz), 7.23 (1H, m), 7.47-7.58 (2H, m), 7.71 (1H, dd, J=7.5, 7.5 Hz), 8.59 (1H, brd, J=4 Hz);

MASS (ES+): m/e 650.55;

[α]_{D}²⁵=−89.0° (c=0.41, CHCl₃).

### EXAMPLE 237

Compound E237 was obtained from the Compound (422) in a manner similar to Example 1.

¹H-NMR (300 MHz, CDCl₃, δ): 0.84 (3H, t, J=7.3 Hz), 1.09 (3×3H, s), 1.23 (3H, d, J=6.6 Hz), 1.29 (3H, s), 1.38-1.52 (2H, m), 1.57-1.92 (4H, m), 2.08-2.41 (6H, m), 2.13 (3H, s), 2.96 (1H, dd, J=13.5, 6 Hz), 3.24 (1H, dd, J=13.5, 9.5 Hz), 3.30 (1H, m), 3.88 (1H, m), 4.22 (1H, m), 4.27 (1H, q, J=6.6 Hz), 4.68 (1H, m), 5.05 (1H, brs), 5.19 (1H, ddd, J=10.3, 9.5, 6 Hz), 5.35 (1H, s), 5.91 (1H, s), 6.62 (1H, d, J=16 Hz), 6.87 (1H, dt, J=16, 7 Hz), 7.14 (1H, d, J=10.5 Hz), 7.19 (2×1H, d, J=8 Hz), 7.31-7.48 (8H, m), 7.55 (1H, d, J=10.3 Hz), 7.55-7.70 (4H, m);

MASS (ES+): m/e 819.44.

### EXAMPLE 238

Compound E238 was obtained from the Compound E237 in a manner similar to Example 3.

¹H-NMR (300 MHz, CDCl₃, δ): 0.82 (3H, t, J=7.3 Hz), 1.10 (3×3H, s), 1.16-1.32 (4H, m), 1.18 (3H, d, J=6.6 Hz), 1.21 (2×3H, d, J=7 Hz), 1.28 (3H, s), 1.38-1.65 (3H, m), 1.68-1.88 (3H, m), 2.08-2.40 (4H, m), 2.51 (2H, m), 2.86 (1H, qq, J=7, 7 Hz), 2.93 (1H, dd, J=13.8, 6.3 Hz), 3.21 (1H, dd, J=13.8, 9.5 Hz), 3.29 (1H, m), 3.86 (1H, m), 4.19 (1H, m), 4.19 (1H, q, J=6.6 Hz), 4.68 (1H, dd, J=8, 2 Hz), 5.18 (1H, ddd, J=10.3, 9.5, 6.3 Hz), 5.90 (1H, s), 7.05-7.18 (4H, m), 7.09 (1H, d, J=10.2 Hz), 7.33-7.48 (6H, m), 7.56 (1H, d, J=10.3 Hz), 7.59-7.67 (4H, m);

MASS (ES+): m/e 823.51.

### EXAMPLE 239

Compound E239 was obtained from the Compound E238 in a manner similar to Example 6.

¹H-NMR (300 MHz, CDCl₃, δ): 0.82 (3H, t, J=7.3 Hz), 1.21 (2×3H, d, J=7 Hz), 1.24-1.42 (4H, m), 1.54-1.90 (6H, m), 2.08-2.59 (6H, m), 2.85 (1H, qq, J=7, 7 Hz), 2.93 (1H, dd, J=14, 6 Hz), 3.20 (1H, dd, J=14, 10 Hz), 3.55 (1H, d, J=5 Hz), 3.87 (1H, m), 4.14-4.29 (2H, m), 4.68 (1H, dd, J=8, 2 Hz), 5.18 (1H, ddd, J=10.3, 10, 6 Hz), 5.85 (1H, s), 7.05-7.20 (5H, m), 7.53 (1H, d, J=10.3 Hz);

MASS (ES+): m/e 585.46;

[α]_{D}²⁵=−124.5° (c=0.27, CHCl₃).

### EXAMPLE 240

Compound E240 was obtained from the Compound (426) in a manner similar to Example 1.

¹H-NMR (300 MHz, CDCl₃, δ): 0.08 (2×3H, s), 0.84 (3H, t, J=7.3 Hz), 0.93 (3×3H, s), 1.09 (3×3H, s), 1.24 (3H, d, J=6.8 Hz), 1.29 (3H, s), 1.38-1.51 (2H, m), 1.54-1.91 (4H, m), 2.08-2.40 (6H, m), 2.94 (1H, dd, J=13.5, 6 Hz), 3.23 (1H, dd, J=13.5, 9.5 Hz), 3.26 (1H, m), 3.86 (1H, m), 4.21 (1H, dt, J=10.2, 7.7 Hz), 4.27 (1H, q, J=6.8 Hz), 4.66 (1H, dd, J=8, 2 Hz), 4.69 (1H, s), 5.18 (1H, ddd, J=10.2, 9.5, 6 Hz), 5.86 (1H, s), 6.62 (1H, d, J=15.8 Hz), 6.87 (1H, dt, J=15.8, 7 Hz), 7.14 (1H, d, J=10.2 Hz), 7.19 (2×1H, d, J=8.5 Hz), 7.23 (2×1H, d, J=8.5 Hz), 7.31-7.48 (6H, m), 7.53 (1H, d, J=10.2 Hz), 7.56-7.69 (4H, m);

MASS (ES+): m/e 923.66.

### EXAMPLE 241

Compound E241 was obtained from the Compound E240 in a manner similar to Example 3.

¹H-NMR (300 MHz, CDCl₃, δ): 0.83 (3H, t, J=7.3 Hz), 1.10 (3×3H, s), 1.19 (3H, d, J=6.6 Hz), 1.20-1.32 (4H, m), 1.28 (3H, s), 1.36-1.88 (6H, m), 2.07-2.40 (4H, m), 2.51 (2H, m), 2.96 (1H, dd, J=14, 6 Hz), 3.23 (1H, dd, J=14, 9.5 Hz), 3.28 (1H, m), 3.86 (1H, m), 4.12-4.25 (2H, m), 4.65 (2H, s), 4.67 (1H, m), 5.18 (1H, m), 5.92 (1H, s), 7.07 (1H, d, J=10.3 Hz), 7.23 (2×1H, d, J=8 Hz), 7.28 (2×1H, d, J=8 Hz), 7.33-7.49 (6H, m), 7.56-7.70 (5H, m);

MASS (ES+): m/e 811.55.

### EXAMPLE 242

Compound E242 was obtained from the Compound E241 in a manner similar to Example 6.

¹H-NMR (300 MHz, CDCl₃, δ): 0.84 (3H, t, J=7.3 Hz), 1.22-1.42 (4H, m), 1.28 (3H, s), 1.38 (3H, d, J=7 Hz), 1.54-1.90 (6H, m), 2.06-2.57 (6H, m), 2.96 (1H, dd, J=13.5, 6.2 Hz), 3.23 (1H, dd, J=13.5, 9.5 Hz), 3.28 (1H, m), 3.56 (1H, d, J=4.5 Hz), 3.86 (1H, m), 4.14-4.28 (2H, m), 4.66 (2H, s), 4.68 (1H, m), 5.18 (1H, ddd, J=10.3, 9.5, 6.2 Hz), 5.92 (1H, s), 7.10 (1H, d, J=10 Hz), 7.23 (2×1H, d, J=8 Hz), 7.28 (2×1H, d, J=8 Hz), 7.57 (1H, d, J=10.3 Hz);

MASS (ES+): m/e 573.57;

[α]_{D}²⁵=−117.8° (c=0.25, CHCl₃).

### EXAMPLE 243

Compound E243 was obtained from the Compound (438) in a manner similar to Example 1.

¹H-NMR (300 MHz, CDCl₃, δ): 0.70-0.84 (6H, m), 0.96-1.96 (12H, m), 1.09 (3×3H, s), 1.24 (3H, d, J=7 Hz), 1.38 (3H, t, J=7 Hz), 2.23 (2H, m), 2.46 (1H, m), 2.68 (1H, m), 2.80 (1H, dd, J=13.5, 6 Hz), 3.18 (1H, dd, J=13.5, 9.5 Hz), 3.97 (2H, q, J=7 Hz), 4.28 (1H, q, J=7 Hz), 4.42-4.63 (4H, m), 4.82 (1H, m), 5.81-5.94 (2H, br), 6.14 (1H, d, J=9.5 Hz), 6.61 (1H, d, J=16 Hz), 6.77 (2×1H, d, J=9 Hz), 6.85 (1H, dt, J=16, 7 Hz), 7.10 (2×1H, d, J=9 Hz), 7.30-7.48 (6H, m), 7.51-7.74 (4H, m);

MASS (ES+): m/e 851.54.

### EXAMPLE 244

Compound E244 was obtained from the Compound E243 in a manner similar to Example 3.

¹H-NMR (300 MHz, CDCl₃, δ): 0.74 (3H, m), 0.79 (3H, d, J=7 Hz), 1.04-1.96 (16H, m), 1.10 (3×3H, s), 1.18 (3H, d, J=7 Hz), 1.38 (3H, d, J=7 Hz), 2.41-2.55 (3H, m), 2.71 (1H, m), 2.80 (1H, dd, J=13.5, 6 Hz), 3.18 (1H, dd, J=13.5, 9.5 Hz), 3.97 (1H, q, J=7 Hz), 4.18 (1H, q, J=7 Hz), 4.41-4.63 (4H, m), 4.83 (1H, m), 5.80-5.98 (2H, m), 6.17 (1H, d, J=11 Hz), 6.76 (2×1H, d, J=8.5 Hz), 7.09 (2×1H, d, J=8.5 Hz), 7.33-7.48 (6H, m), 7.58-7.68 (4H, m);

MASS (ES+): m/e 853.57.

### EXAMPLE 245

Compound E245 was obtained from the Compound E244 in a manner similar to Example 6.

¹H-NMR (300 MHz, CDCl₃, δ): 0.68-0.80 (6H, m), 0.79 (3H, d, J=6.5 Hz), 1.11 (1H, m), 1.21-1.98 (15H, m), 1.38 (3H, d, J=7 Hz), 1.39 (3H, t, J=7 Hz), 2.34-2.58 (3H, m), 2.71 (1H, m), 2.80 (1H, dd, J=13.5, 6 Hz), 3.18 (1H, dd, J=13.5, 9.5 Hz), 3.56 (1H, d, J=5 Hz), 3.97 (2H, q, J=7 Hz), 4.23 (1H, m), 4.42-4.63 (4H, m), 4.84 (1H, m), 5.93-6.05 (2H, m), 6.20 (1H, d, J=10.5 Hz), 6.76 (2×1H, d, J=8.5 Hz), 7.09 (2×1H, d, J=8.5 Hz);

MASS (ES+): m/e 615.62;

[α]_{D}²⁵=−117.8° (c=0.20, CHCl₃).

### EXAMPLE 246

Compound E246 was obtained from the Compound (444) in a manner similar to Example 1.

¹H-NMR (300 MHz, CDCl₃, δ): 1.10 (9H, s), 1.23 (3H, d, J=7 Hz), 1.28 (3H, d, J=7 Hz), 1.40-1.53 (2H, m), 1.61-1.91 (4H, m), 2.12-2.38 (4H, m), 2.93 (1H, dd J=14, 6 Hz), 3.16 (1H, dt, J=10, 7 Hz), 3.22 (1H, dd, J=14, 10 Hz), 3.91 (1H, dt, J=10, 4 Hz), 4.23-4.35 (1H, m), 4.327 (1H, q, J=7 Hz), 4.51-4.68 (2H, m), 5.12 (1H, dt, J=6, 10 Hz), 6.10 (1H, d, J=10 Hz), 6.53 (1H, d, J=10 Hz), 6.61 (1H, d, J=15 Hz), 6.87 (1H, dt, J=15, 8 Hz), 7.14-7.48 (12H, m), 7.57-7.70 (4H, m); MASS: m/z 751.28 (M+H)⁺.

### EXAMPLE 247

Compound E247 was obtained from the Compound E246 in a manner similar to Example 3.

¹H-NMR (300 MHz, CDCl₃, δ): 1.10 (9H, s), 1.15-1.35 (2H, m), 1.18 (3H, d, J=7 Hz), 1.28 (3H, d, J=7 Hz), 1.37-1.50 (2H, m), 1.55-1.90 (6H, m), 2.14-2.41 (2H, m), 2.51 (2H, t, J=7 Hz), 2.93 (1H, dd J=14, 6 Hz), 3.17 (1H, dt, J=10, 7 Hz), 3.22 (1H, dd, J=14, 10 Hz), 3.90 (1H, dt, J=10, 4 Hz), 4.18 (1H, q, J=7 Hz), 4.25 (1H, J=10, 7 Hz), 4.52-4.68 (2H, m), 5.12 (1H, dt, J=6, 10 Hz), 6.09 (1H, d, J=10 Hz), 6.55 (1H, d, J=10 Hz), 7.11 (1H, d J=10 Hz), 7.18-7.33 (5H, m), 7.33-7.50 (6H, m), 7.59-7.74 (4H, m).

### EXAMPLE 248

Compound E248 was obtained from the Compound E247 in a manner similar to Example 6.

¹H-NMR (300 MHz, CDCl₃, δ): 1.22-1.42 (4H, m), 1.28 (3H, d, J=7 Hz), 1.38 (3H, d, J=7 Hz), 1.53-1.91 (6H, m), 2.10-2.59 (2H, m), 2.47 (2H, dt, J=13, 7 Hz), 2.93 (1H, dd J=14, 6 Hz), 3.16 (1H, dt, J=10, 7 Hz), 3.21 (1H, dd, J=14, 10 Hz), 3.57 (1H, d, J=5 Hz), 3.90 (1H, dt, J=10, 4 Hz), 4.19-4.33 (2H, m), 4.51-4.69 (2H, m), 5.11 (1H, dt, J=6, 10 Hz), 6.15 (1H, d, J=10 Hz), 6.55 (1H, d, J=10 Hz), 7.15 (1H, d, J=10 Hz), 7.18-7.36 (5H, m).

### EXAMPLE 249

Compound E249 was obtained from the Compound (461) in a manner similar to Example 1.

¹H-NMR (300 MHz, CDCl₃, δ): 1.09 (9H, s), 1.22 (3H, d, J=7.0 Hz), 1.38-1.73 (8H, m), 1.74 (3H, s), 1.95-2.30 (2H, m), 2.89-3.00 (1H, m), 2.95 (1H, d, J=13.6 Hz), 3.08-3.30 (2H, m), 3.16 (1H, d, J=13.6 Hz), 3.69-3.83 (1H, m), 4.06-4.21 (1H, m), 4.27 (1H, q, J=7.0 Hz), 4.57-4.66 (1H, m), 5.08-5.20 (1H, m), 6.08 (1H, s), 6.57 (1H, d, J=15.4 Hz), 6.84 (1H, dt, J=15.4, 7.0 Hz), 7.05 (1H, d, J=10.6 Hz), 7.16-7.47 (17H, m), 7.59 (1H, d, J=7.7 Hz), 7.59 (1H, d, J=8.1 Hz), 7.65 (1H, d, J=7.7 Hz), 7.65 (1H, d, J=8.1 Hz);

MASS (ES+): m/e 841.23 (M+1).

### EXAMPLE 250

Compound E250 was obtained from the Compound E249 in a manner similar to Example 3.

¹H-NMR (300 MHz, CDCl₃, δ): 1.10 (9H, s), 1.13-1.83 (10H, m), 1.18 (3H, d, J=6.6 Hz), 1.73 (3H, s), 2.00-2.16 (1H, m), 2.19-2.31 (1H, m), 2.43-2.53 (2H, m), 2.87-3.00 (1H, m), 2.94 (1H, d, J=13.5 Hz), 3.10-3.34 (2H, m), 3.15 (1H, d, J=13.5 Hz), 3.71-3.81 (1H, m), 4.06-4.19 (1H, m), 4.18 (1H, q, J=6.6 Hz), 4.58-4.66 (1H, m), 5.09-5.19 (1H, m), 6.05 (1H, s), 6.99 (1H, d, J=10.3 Hz), 7.17-7.48 (17H, m), 7.61 (2H, d, J=8.1 Hz), 7.64 (2H, d, J=8.1 Hz);

MASS (ES+): m/e 843.28 (M+1).

### EXAMPLE 251

Compound E251 was obtained from the Compound E250 in a manner similar to Example 6.

¹H-NMR (300 MHz, CDCl₃, δ): 1.14-1.83 (10H, m), 1.38 (3H, d, J=7.0 Hz), 1.73 (3H, s), 1.97-2.15 (1H, m), 2.16-2.31 (1H, m), 2.34-2.56 (2H, m), 2.89-3.00 (1H, m), 2.94 (1H, d, J=13.9 Hz), 3.08-3.30 (2H, m), 3.15 (1H, d, J=13.9 Hz), 3.55 (1H, d, J=4.4 Hz), 3.71-3.82 (1H, m), 4.07-4.28 (2H, m), 4.58-4.67 (1H, m), 5.07-5.21 (1H, m), 6.04 (1H, s), 7.01 (1H, d, J=9.5 Hz), 7.16-7.43 (10H, m), 7.38 (1H, d, J=10.3 Hz);

MASS (ES+): m/e 605.37 (M+1).

### EXAMPLE 252

Compounds E23 (main product) and E252 (by-product) were obtained from the Compound E22 in a manner similar to Example 6.

¹H-NMR (300 MHz, CDCl₃, δ): (for Compound E23) 0.81 (3H, t, J=7.3 Hz), 1.22-1.41 (2H, m), 1.28 (3H, s), 1.38 (3H, d, J=7.3 Hz), 1.54-1.92 (8H, m), 2.06-2.57 (6H, m), 3.06 (1H, dd, J=13.9, 7.0 Hz), 3.24-3.36 (1H, m), 3.26 (1H, dd, J=13.9, 8.8 Hz), 3.55 (1H, d, J=4.8 Hz), 3.79-3.90 (2H, m), 4.15-4.29 (2H, m), 4.65-4.72 (1H, m), 5.18 (1H, ddd, J=10.3, 8.8, 7.0 Hz), 5.89 (1H, s), 6.99 (1H, d, J=10.3 Hz), 7.58 (2H, d, J=8.4 Hz), 7.35 (2H, d, J=8.4 Hz), 7.64 (1H, d, J=10.3 Hz);

MASS (ES+): m/e 568.42 (M+1).

¹H-NMR (300 MHz, CDCl₃, δ): (for Compound E252) 0.83 (3H, t, J=7.3 Hz), 1.22-1.41 (2H, m), 1.28 (3H, s), 1.38 (3H, t, J=7.0 Hz), 1.50-1.96 (8H, m), 2.08-2.40 (4H, m), 2.47 (2H, dt, J=12.5, 7.3 Hz), 3.03 (1H, dd, J=13.5, 6.2 Hz), 3.22-3.33 (1H, m), 3.25 (1H, dd, J=13.5, 9.2 Hz), 3.80-3.89 (1H, m), 3.90 (1H, s), 4.17-4.30 (1H, m), 4.24 (1H, q, J=7.0 Hz), 4.64-4.70 (1H, m), 5.19 (1H, ddd, J=10.3, 9.2, 6.2 Hz), 5.85 (1H, s), 7.06 (1H, d, J=10.3 Hz), 7.31 (2H, d, J=8.4 Hz), 7.60 (1H, d, J=10.3 Hz), 7.95 (2H, d, J=8.4 Hz);

MASS (ES+): m/e 601.46 (M+1).

### EXAMPLE 253

Compounds E252 (20 mg) was dissolved in methanol (0.3 ml) and the mixture was stirred at ambient temperature. To the mixture was added a 40% solution of N-methylamino metanol in methanol and the mixture was stirred under ambient temperature for 4 hours. The solvent and the residual agents were removed by evaporation, and the residue was purified by preparative chromatography (eluting with ethyl acetate/methanol=9/1) to give the objective Compound E253.

¹H-NMR (300 MHz, CDCl₃, δ): 0.83 (3H, t, J=7.3 Hz), 1.20-1.41 (2H, m), 1.28 (3H, s), 1.38 (3H, d, J=7.3 Hz), 1.49-1.89 (8H, m), 1.98-2.40 (4H, m), 2.47 (2H, dt, J=11.7, 7.3 Hz), 3.00 (1H, dd, J=13.5, 6.2 Hz), 3.21-3.32 (1H, m), 3.27 (1H, dd, J=13.5, 9.5 Hz), 3.53-3.59 (1H, m), 3.79-3.90 (1H, m), 4.14-4.29 (2H, m), 4.63-4.69 (1H, m), 5.18 (1H, ddd, J=10.3, 9.5, 6.2 Hz), 5.90 (1H, s), 6.05-6.14 (1H, m), 7.06 (1H, d, J=10.3 Hz), 7.30 (2H, d, J=8.4 Hz), 7.60 (1H, d, J=10.3 Hz), 7.67 (2H, d, J=8.4 Hz);

MASS (ES+): m/e 600.55 (M+1).

### EXAMPLE 254

Compound E254 was obtained from the Compound (453) in a manner similar to Example 1.

¹H-NMR (300 MHz, CDCl₃, δ): 0.81 (3H, t, J=7.7 Hz), 1.09 (9H, s), 1.22 (3H, d, J=7.0 Hz), 1.28 (3H, s), 1.38-1.90 (8H, m), 2.09-2.42 (4H, m), 3.04 (1H, dd, J=13.6, 6.2 Hz), 3.22-3.38 (1H, m), 3.31 (1H, dd, J=13.6, 9.9 Hz), 3.81-3.93 (1H, m), 4.17-4.33 (1H, m), 4.27 (1H, q, J=7.0 Hz), 4.65-4.72 (1H, m), 5.13-5.26 (1H, m), 5.83 (1H, s), 6.62 (1H, d, J=15.4 Hz), 6.88 (1H, dt, J=15.4, 7.0 Hz), 7.04 (1H, d, J=10.3 Hz), 7.30-7.48 (7H, m), 7.51-7.76 (8H, m);

MASS (ES+): m/e 847.18 (M+1).

### EXAMPLE 255

Compound E255 was obtained from the Compound E254 in a manner similar to Example 3.

¹H-NMR (300 MHz, CDCl₃, δ): 0.82 (3H, t, J=7.3 Hz), 1.11 (9H, s), 1.18 (3H, d, J=6.6 Hz), 1.28 (3H, s), 1.38-1.90 (8H, m), 2.09-2.41 (4H, m), 2.46-2.57 (2H, m), 3.04 (1H, dd, J=13.6, 6.6 Hz), 3.22-3.36 (1H, m), 3.28 (1H, dd, J=13.6, 9.9 Hz), 3.80-3.92 (1H, m), 4.10-4.27 (1H, m), 4.19 (1H, q, J=6.6 Hz), 4.65-4.72 (1H, m), 5.13-5.27 (1H, m), 5.82 (1H, s), 7.00 (1H, d, J=10.3 Hz), 7.32-7.49 (7H, m), 7.54 (2H, d, J=8.4 Hz), 7.58-7.69 (5H, m), 7.69-7.76 (1H, m);

MASS (ES+): m/e 849.25 (M+1).

### EXAMPLE 256

Compound E256 was obtained from the Compound E255 in a manner similar to Example 6.

¹H-NMR (300 MHz, CDCl₃, δ): 0.82 (3H, s), 1.17-1.43 (2H, m), 1.28 (3H, s), 1.38 (3H, d, J=7.3 Hz), 1.48-1.92 (8H, m), 2.06-2.59 (6H, m), 3.04 (1H, dd, J=13.9, 6.6 Hz), 3.22-3.37 (1H, m), 3.28 (1H, dd, J=13.9, 9.5 Hz), 3.57 (1H, d, J=4.8 Hz), 3.80-3.93 (1H, m), 4.15-4.30 (2H, m), 4.63-4.74 (1H, m), 5.13-5.28 (1H, m), 5.86 (1H, s), 7.03 (1H, d, J=9.9 Hz), 7.35 (2H, d, J=8.1 Hz), 7.54 (2H, d, J=8.1 Hz), 7.62 (1H, d, J=10.3 Hz);

MASS (ES+): m/e 611.30 (M+1);

[α]_{D}²⁵=−98.9° (c=0.475).

### EXAMPLE 257

Compound E257 was obtained from the Compound (469) in a manner similar to Example 1.

¹H-NMR (300 MHz, CDCl₃, δ): 1.10 (9H, s), 1.17 (3H, s), 1.24 (3H, d, J=7.0 Hz), 1.41-1.99 (6H, m), 2.11-2.39 (4H, m), 3.09 (1H, dd, J=13.6, 6.6 Hz), 3.28-3.39 (1H, m), 3.29 (1H, dd, J=13.6, 8.8 Hz), 3.42 (1H, d, J=13.6 Hz), 3.60 (1H, d, J=13.6 Hz), 3.82-3.92 (1H, m), 4.16-4.25 (1H, m), 4.29 (1H, q, J=7.0 Hz), 4.66-4.73 (1H, m), 5.21-5.34 (1H, m), 5.91 (1H, s), 6.63 (1H, d, J=15.7 Hz), 6.89 (1H, dt, J=15.7, 6.6 Hz), 6.97-7.04 (2H, m), 7.13-7.21 (4H, m), 7.22-7.48 (11H, m), 7.57-7.69 (4H, m), 7.84 (1H, d, J=10.3 Hz);

MASS (ES+): m/e 841.21 (M+1).

### EXAMPLE 258

Compound E258 was obtained from the Compound E257 in a manner similar to Example 3.

¹H-NMR (300 MHz, CDCl₃, δ): 1.10 (9H, s), 1.17 (3H, s), 1.19 (3H, d, J=7.0 Hz), 1.41-1.69 (6H, m), 1.71-1.95 (4H, m), 2.09-2.40 (2H, m), 2.53 (2H, t, J=7.3 Hz), 3.08 (1H, dd, J=13.9, 7.0 Hz), 3.23-3.36 (1H, m), 3.29 (1H, dd, J=13.9, 9.1 Hz), 3.36 (1H, d, J=13.9 Hz), 3.64 (1H, d, J=13.9 Hz), 3.78-3.91 (1H, m), 4.12-4.23 (1H, m), 4.20 (1H, q, J=7.0 Hz), 4.64-4.73 (1H, m), 5.21-5.32 (1H, m), 5.86 (1H, s), 6.97-7.06 (2H, m), 7.11 (1H, d, J=10.3 Hz), 7.15-7.47 (14H, m), 7.56-7.68 (4H, m, J=10.3 Hz), 7.86 (1H, d, J=10.3 Hz);

MASS (ES+): m/e 843.18 (M+1).

### EXAMPLE 259

Compound E259 was obtained from the Compound E258 in a manner similar to Example 6.

¹H-NMR (300 MHz, CDCl₃, δ): 1.17 (3H, s), 1.29-1.42 (3H, m), 1.39 (1H, d, J=7.3 Hz), 1.51-1.73 (4H, m), 1.73-1.98 (3H, m), 2.08-2.60 (4H, m), 3.09 (1H, dd, J=13.9, 7.0 Hz), 3.27-3.39 (1H, m), 3.29 (1H, dd, J=13.9, 8.8 Hz), 3.37 (1H, d, J=13.5 Hz), 3.56 (1H, d, J=4.0 Hz), 3.65 (1H, d, J=13.5 Hz), 3.81-3.92 (1H, m), 4.14-4.29 (2H, m), 4.67-4.74 (1H, m), 5.21-5.33 (1H, m), 5.87 (1H, s), 6.99-7.06 (2H, m), 7.14-7.35 (8H, m), 7.14 (1H, d, J=10.6 Hz), 7.85 (1H, d, J=10.3 Hz);

MASS (ES+): m/e 605.38 (M+1);

[α]_{D}²⁶=−148.2° (c=0.55).

### EXAMPLE 260

Compound E260 was obtained from the Compound (105) in a manner similar to Example 1.

¹H-NMR (300 MHz, CDCl₃, δ): 0.81 (3H, t, J=7.3 Hz), 1.27 (3H, s), 1.30-1.55 (1H, m), 1.41 (6H, s), 1.56-1.92 (6H, m), 2.24-2.38 (6H, m), 2.02 (1H, s), 2.07-2.38 (6H, m), 2.96 (1H, dd, J=13.6, 2.2 Hz), 3.23 (1H, dd, J=13.6, 9.2 Hz), 3.20-3.32 (1H, m), 3.80-3.90 (1H, m), 4.20 (1H, ddd, J=15.4, 7.7, 7.7 Hz), 4.34 (2H, s), 4.63-4.69 (1H, brd, J=5.5 Hz), 5.18 (1H, ddd, J=17.6, 11.0, 7.7 Hz), 5.87 (1H, s), 6.79 (1H, d, J=15.4 Hz), 7.02 (1H, ddd, J=15.4, 6.6, 6.6 hz), 7.12 (1H, d, J=10.3 Hz);

MASS (ES+): m/e 667.3 (M+Na).

### EXAMPLE 261

Compound E261 was obtained from the Compound E260 in a manner similar to Example 3.

1H-NMR (300 MHz, CDCl₃, δ): 1.28 (3H, t, J=7.3 Hz), 1.73 (3H, s), 1.65-1.87 (5H, m), 1.82 (6H, s), 2.00-2.34 (6H, m), 2.52-2.70 (2H, m), 2.70-2.85 (2H, m), 2.99 (2H, t, J=7.3 Hz), 3.41 (1H, dd, J=13.6, 6.2 Hz), 3.61-3.78 (2H, m), 4.23-4.39 (1H, m), 4.66 (1H, ddd, J=17.6, 7.7 Hz), 5.12 (1H, brd, J=6.2 Hz), 5.63 (1H, ddd, J=17.2, 14.3, 7.3 Hz), 6.40 (1H, s), 7.53-7.89 (6H, m, J=8 Hz), 8.02 (1H, brd, J=10.3 Hz);

MASS (ES+): m/e 557.3 (M+1).

### EXAMPLE 262

Compound E262 was obtained from the Compound (105) in a manner similar to Example 1.

¹H-NMR (300 MHz, CDCl₃, δ): 0.83 (3H, t, J=7.3 Hz), 1.10 (3H, t, J=7.3 Hz), 1.28 (3H, s), 1.37-1.56 (2H, m), 1.56-1.94 (4H, m), 2.00-2.40 (6H, m), 2.56 (2H, q, J=7.3 Hz), 2.96 (1H, dd, J=13.6, 6.2 Hz), 3.16-3.33 (2H, m), 3.80-3.93 (1H, m), 4.23 (1H, ddd, J=15.8, 7.7, 7.7 Hz), 4.68 (1H, brd, J=5.9 Hz), 5.19 (1H, ddd, J=16.1, 9.9, 6.2 Hz), 5.92 (1H, s), 6.10 (1H, d, J=16.1 Hz), 6.79 (1H, dt, J=15.8, 7.0 Hz), 7.10-7.33 (6H, m), 7.54 (1H, brd, J=10.3 Hz);

MASS (ES+): m/e 525.7 (M+1).

### EXAMPLE 263

Compound E263 was obtained from the Compound (374) in a manner similar to Example 1.

¹H-NMR (300 MHz, CDCl₃, δ): 0.83 (3H, t, J=7.3 Hz), 1.28 (3H, s), 1.43-1.58 (2H, m), 1.59-1.91 (3H, m), 2.10-2.37 (4H, m), 2.96 (1H, dd, J=13.6, 6.2 Hz), 3.16-3.37 (2H, m), 3.85 (1H, ddd, J=10.3, 10.3, 5.1 Hz), 4.25 (1H, ddd, J=10.3, 7.7, 7.7 Hz), 4.69 (1H, brd, J=5.5 Hz), 4.95 (2H, d, J=47.6 Hz), 5.17 (1H, ddd, J=16.5, 9.5, 6.5 Hz), 6.07 (1H, s), 6.35 (1H, brd, J=15.8 Hz), 6.83-7.08 (4H, m), 7.10-7.31 (5H, m), 7.58 (1H, d, J=9.9 Hz);

MASS (ES+): m/e 547.3 (M+1).

### EXAMPLE 264

Compound E264 was obtained from the Compound E263 in a manner similar to Example 3.

¹H-NMR (300 MHz, CDCl₃, δ): 0.83 (3H, t, J=7.3 Hz), 1.21-1.42 (4H, m), 1.28 (3H, s), 1.53-1.70 (2H, m), 1.70-1.92 (4H, m), 2.08-2.39 (4H, m), 2.54 (2H, ddd, J=7.3, 7.3, 2.6 Hz), 2.96 (1H, dd, J=13.5, 6.2 Hz), 3.15-3.36 (2H, m), 3.86 (1H, ddd, J=10.2, 8.4, 4.8 Hz), 4.22 (1H, ddd, J=10.2, 7.7, 7.7 Hz), 4.69 (1H, brd, J=5.9 Hz), 4.80 (2H, d, J=47.6 Hz), 5.16 (1H, ddd, J=9.2, 9.2, 6.2 Hz), 6.00 (1H, s), 6.86-7.13 (5H, m), 7.19-7.28 (1H, m), 7.61 (1H, d, J=10.3 Hz);

MASS (ES+): m/e 549.4 (M+1).

### EXAMPLE 265

Compound E265 was obtained from the Compound (374) in a manner similar to Example 1.

¹H-NMR (300 MHz, CDCl₃, δ): 0.83 (3H, t, J=7.3 Hz), 1.21-1.40 (4H, m), 1.28 (3H, s), 1.47-1.68 (3H, m), 1.69-1.90 (3H, m), 2.10-2.46 (4H, m), 2.13 (3H, s), 2.54 (2H, ddd, J=7.3, 7.3, 2.9 Hz), 2.96 (1H, dd, J=13.5, 6.6 Hz), 3.15-3.35 (2H, m), 3.85 (1H, ddd, J=10.3, 10.3, 5.1 Hz), 4.22 (1H, ddd, J=10.3, 10.3, 8.1 Hz), 4.69 (1H, brd, J=6.6 Hz), 5.16 (1H, ddd, J=9.5, 9.5, 6.6 Hz), 6.09 (1H, s), 6.85-7.15 (4H, m), 7.18-7.28 (1H, m), 7.62 (1H, d, J=10.3 Hz);

MASS (ES+): m/e 531.4 (M+1).

### EXAMPLE 266

Compound E266 was obtained from the Compound (477) in a manner similar to Example 1.

¹H-NMR (300 MHz, CDCl₃, δ): 0.83 (1H, t, J=7.3 Hz), 1.09 (4H, s), 1.10 (5H, s), 1.22 (3H, d, J=6.6 Hz), 1.29 (3H, s), 1.38-19.2 (8H, m), 2.04-2.38 (6H, m), 2.93 (1H, dd, J=13.9, 6.6 Hz), 3.1.8 (1H, dd, J=13.9, 9.2 Hz), 3.30 (1H, dt, J=10.3, 7.3 Hz), 3.80-3.90 (1H, m), 4.17-4.31 (2H, m), 4.67-4.71 (1H, m), 5.12 (1H, ddd, J=9.8, 9.2, 6.6 Hz), 5.95 (1H, s), 6.61 (1H, d, J=15.5 Hz), 6.8 (1H, dt, J=15.5, 6.6 Hz), 6.91-6.98 (1H, m), 7.01-7.12 (3H, m), 7.31-7.49 (6H, m), 7.55-7.70 (5H, m);

MASS (ES+): m/e 815.46 (M+1).

### EXAMPLE 267

Compound E267 was obtained from the Compound E266 in a manner similar to Example 3.

¹H-NMR (300 MHz, CDCl₃, δ): 0.83 (3H, t, J=7.3 Hz), 1.10 (9H, s), 1.18 (3H, d, J=7.0 Hz), 1.29 (3H, s), 1.36-1.91 (10H, m), 2.08-2.38 (4H, m), 2.51 (2H, dt, J=7.0, 2.6 Hz), 2.92 (1H, dd, J=13.5, 6.2 Hz), 3.18 (1H, dd, J=13.5, 9.5 Hz), 3.30 (1H, dt, J=10.3, 8.0 Hz), 3.80-3.89 (1H, m), 4.14-4.27 (1H, m), 4.19 (1H, q, J=7.0 Hz), 4.66-4.72 (1H, m), 5.11 (1H, ddd, J=9.5, 9.2, 6.6 Hz), 5.82 (1H, s), 6.91-6.99 (1H, m), 7.00-7.11 (3H, m), 7.33-7.48 (6H, m), 7.58-7.67 (5H, m);

MASS (ES+): m/e 817.45 (M+1).

### EXAMPLE 268

Compound E268 was obtained from the Compound E267 in a manner similar to Example 6.

¹H-NMR (300 MHz, CDCl₃, δ): 0.83 (3H, t, J=7.3 Hz), 1.23-1.41 (5H, m), 1.29 (3H, s), 1.52-1.97 (8H, m), 2.06-2.58 (6H, m), 2.92 (1H, dd, J=13.6, 6.6 Hz), 3.18 (1H, dd, J=13.6, 9.2 Hz), 3.31 (1H, dt, J=10.3, 7.3 Hz), 3.59 (1H, d, J=4.0 Hz), 3.80-3.90 (1H, m), 4.16-4.29 (2H, m), 4.65-4.73 (1H, m), 5.12 (1H, ddd, J=9.9, 9.5, 6.6 Hz), 5.92 (1H, s), 6.91-6.99 (1H, m), 7.00-7.12 (3H, m), 7.60 (1H, d, J=10.3 Hz);

MASS (ES+): m/e 579.55 (M+1).

### EXAMPLE 269

Compound E269 was obtained from the Compound (483) in a manner similar to Example 1.

¹H-NMR (300 MHz, CDCl₃, δ): 0.83 (3H, t, J=7.3 Hz), 1.09 (3×3H, s), 1.18-1.92 (12H, m), 1.22 (3H, d, J=7 Hz), 1.28 (3H, s), 2.08-2.40 (6H, m), 2.58 (2H, m), 2.86-2.98 (3H, m), 3.21 (1H, dd, J=13.5, 9.5 Hz), 3.23-3.37 (3H, m), 3.55 (2H, m), 3.88 (1H, m), 4.21 (1H, m), 4.27 (1H, q, J=7 Hz), 4.68 (1H, dd, J=7.5, 2 Hz), 5.17 (1H, m), 5.89 (1H, s), 6.62 (1H, d, J=15.8 Hz), 6.87 (1H, dt, J=15.8, 7 Hz), 7.08-7.18 (5H, m), 7.31-7.48 (6H, m), 7.53 (1H, d, J=10 Hz), 7.56-7.69 (4H, m);

MASS (ES+): m/e 918.56.

### EXAMPLE 270

Compound E270 was obtained from the Compound E269 in a manner similar to Example 3.

¹H-NMR (300 MHz, CDCl₃, δ): 0.83 (3H, t, J=7.3 Hz), 1.10 (3×3H, s), 1.18 (3H, d, J=7 Hz), 1.18-1.32 (4H, m), 1.28 (3H, s), 1.39-1.89 (12H, m), 2.08-2.40 (4H, m), 2.46-2.62 (4H, m), 2.86-2.98 (3H, m), 3.20 (1H, dd, J=13.5, 9.5 Hz), 3.22-3.37 (3H, m), 3.55 (2H, m), 3.86 (1H, m), 4.18 (1H, m), 4.18 (1H, q, J=7 Hz), 4.68 (1H, m), 5.16 (1H, m), 5.89 (1H, s), 7.08 (1H, d, J=10.3 Hz), 7.08-7.18 (4H, m), 7.32-7.48 (6H, m), 7.57 (1H, d, J=10.3 Hz), 7.57-7.66 (4H, m);

MASS (ES−): m/e 954.65 (M+Cl).

### EXAMPLE 271

Compound E271 was obtained from the Compound E270 in a manner similar to Example 6.

¹H-NMR (300 MHz, CDCl₃, δ): 0.84 (3H, t, J=7.3 Hz), 1.20-1.40 (4H, m), 1.28 (3H, s), 1.38 (3H, d, J=7 Hz), 1.42-1.89 (12H, m), 2.07-2.62 (8H, m), 2.83-2.97 (3H, m), 3.20 (1H, dd, J=13.5, 9.5 Hz), 3.23-3.37 (3H, m), 3.50-3.59 (3H, m), 3.87 (1H, m), 4.14-4.28 (2H, m), 4.68 (1H, m), 5.16 (1H, m), 5.91 (1H, s), 7.07-7.19 (5H, m), 7.55 (1H, d, J=10 Hz);

MASS (ES+): m/e 682.57.

### EXAMPLE 272

Compound E272 was obtained from the Compound (486) in a manner similar to Example 1.

¹H-NMR (300 MHz, CDCl₃, δ): 0.82 (3H, t, J=7.3 Hz), 1.09 (3×3H, s), 1.22 (3H, t, J=7 Hz), 1.27 (3H, s), 1.38-1.51 (2H, m), 1.56-1.91 (4H, m), 2.07-2.38 (6H, m), 2.63 (2H, t, J=7.5 Hz), 2.94 (1H, dd, J=14, 6.5 Hz), 3.01 (2H, t, J=7.5 Hz), 3.20 (1H, dd, J=14, 9.5 Hz), 3.25 (1H, m), 3.85 (1H, m), 4.21 (1H, m), 4.27 (1H, q, J=7 Hz), 4.66 (1H, m), 5.15 (1H, ddd, J=10, 9.5, 6.5 Hz), 5.89 (1H, s), 6.61 (1H, d, J=16 Hz), 6.86 (1H, dt, J=16, 7 Hz), 7.01-7.21 (7H, m), 7.26-7.49 (8H, m), 7.53 (1H, d, J=10 Hz), 7.57-7.70 (6H, m);

MASS (ES+): m/e 926.49.

### EXAMPLE 273

Compound E273 was obtained from the Compound E272 in a manner similar to Example 3.

¹H-NMR (300 MHz, CDCl₃, δ): 0.82 (3H, t, J=7.4 Hz), 1.10 (3×3H, s), 1.16-1.32 (4H, m), 1.18 (3H, d, J=7 Hz), 1.27 (3H, s), 1.38-1.64 (3H, m), 1.67-1.85 (3H, m), 2.08-2.38 (4H, m), 2.51 (2H, m), 2.63 (2H, t, J=7.3 Hz), 2.94 (1H, dd, J=13.5, 6 Hz), 3.01 (2H, t, J=7.3 Hz), 3.20 (1H, dd, J=13.5, 9.5 Hz), 3.26 (1H, m), 3.84 (1H, m), 4.08-4.24 (2H, m), 4.66 (1H, m), 5.15 (1H, m), 5.88 (1H, s), 7.00-7.20 (7H, m), 7.25-7.50 (10H, m), 7.54-7.70 (5H, m);

MASS (ES+): m/e 928.42.

### EXAMPLE 274

Compound E274 was obtained from the Compound E273 in a manner similar to Example 6.

¹H-NMR (300 MHz, CDCl₃, δ): 0.82 (3H, t, J=7.3 Hz), 1.20-1.41 (4H, m), 1.28 (3H, s), 1.38 (3H, d, J=7 Hz), 1.52-1.88 (6H, m), 2.05-2.57 (6H, m), 2.63 (2H, t, J=7.5 Hz), 2.93 (1H, dd, J=14, 6 Hz), 3.01 (2H, t, J=7.5 Hz), 3.20 (1H, dd, J=14, 9.5 Hz), 3.26 (1H, m), 3.56 (1H, brd, J=5 Hz), 3.84 (1H, m), 4.14-4.28 (2H, m), 4.66 (1H, dd, J=8, 2 Hz), 5.15 (1H, ddd, J=10.3, 9.5, 6 Hz), 5.91 (1H, s), 7.02-7.20 (7H, m), 7.30 (2×1H, dd, J=7.5, 7.5 Hz), 7.43 (2×1H, d, J=7.5 Hz), 7.55 (1H, d, J=10.3 Hz);

MASS (ES+): m/e 690.49;

[α]_{D}²⁵=−104.0° (c=0.21, CHCl₃).

### EXAMPLE 275

Compound E275 was obtained from the Compound (498) in a manner similar to Example 1.

¹H-NMR (300 MHz, CDCl₃, δ): 1.09 (3×3H, s), 1.16-1.86 (8H, m), 1.23 (3H, d, J=7 Hz), 1.90-2.27 (4H, m), 3.01 (1H, m), 3.08 (1H, dd, J=14, 7 Hz), 3.23 (1H, dd, J=13.5, 6 Hz), 3.26 (1H, dd, J=14, 8 Hz), 3.62 (1H, dd, J=13.5, 10.5 Hz), 3.74 (1H, m), 3.96 (1H, m), 4.17 (1H, m), 4.28 (1H, q, J=7 Hz), 5.01 (1H, m), 5.36 (1H, m), 6.45 (1H, d, J=6 Hz), 6.46 (1H, d, J=10 Hz), 6.59 (1H, d, J=15.7, 7 Hz), 6.82 (1H, dt, J=15.7, 7 Hz), 7.06-7.12 (2H, m), 7.15-7.50 (15H, m), 7.56-7.69 (4H, m);

MASS (ES+): m/e 841.34.

### EXAMPLE 276

Compound E276 was obtained from the Compound E275 in a manner similar to Example 3.

¹H-NMR (300 MHz, CDCl₃, δ): 1.10 (3×3H, s), 1.12-1.29 (4H, m), 1.29 (3H, d, J=7 Hz), 1.36-1.81 (8H, m), 1.91-2.18 (2H, m), 2.50 (2H, m), 3.01 (1H, m), 3.08 (1H, dd, J=14, 7 Hz), 3.15-3.30 (2H, m), 3.58-3.77 (2H, m), 3.94 (1H, m), 4.11-4.23 (2H, m), 5.01 (1H, m), 5.35 (1H, m), 6.42 (1H, d, J=6.5 Hz), 6.43 (1H, d, J=9.5 Hz), 7.05-7.13 (2H, m), 7.15-7.49 (15H, m), 7.57-7.67 (4H, m);

MASS (ES+): m/e 843.58.

### EXAMPLE 277

Compound E277 was obtained from the Compound E276 in a manner similar to Example 6.

¹H-NMR (300 MHz, CDCl₃, δ): 1.14-1.40 (6H, m), 1.38 (3H, d, J=7 Hz), 1.44-1.85 (6H, m), 1.97 (1H, m), 2.09 (1H, m), 2.33-2.56 (2H, m), 3.01 (1H, m), 3.08 (1H, dd, J=14, 7 Hz), 3.21 (1H, dd, J=13, 6 Hz), 3.25 (1H, dd, J=14, 8 Hz), 3.58 (1H, d, J=4.5 Hz), 3.64 (1H, dd, J=13, 11 Hz), 3.73 (1H, m), 3.95 (1H, m), 4.10-4.29 (2H, m), 5.02 (1H, m), 5.35 (1H, m), 6.41-6.52 (2H, m), 7.06-7.14 (8H, m), 7.16-7.34 (8H, m), 7.44 (1, d, J=10 Hz);

MASS (ES+): m/e 605.36.

### EXAMPLE 278

Compound E278 was obtained from the Compound (498) in a manner similar to Example 1.

¹H-NMR (300 MHz, CDCl₃, δ): 1.10 (3×3H, s), 1.24 (3H, d, J=7 Hz), 1.29-1.86 (8H, m), 1.90-2.27 (4H, m), 3.01 (1H, m), 3.08 (1H, dd, J=14, 7 Hz), 3.18-3.31 (2H, m), 3.61 (1H, dd, J=13, 10.5 Hz), 3.72 (1H, m), 3.96 (1H, m), 4.16 (1H, m), 4.28 (1H, q, J=7 Hz), 5.01 (1H, m), 5.36 (1H, m), 6.40 (1H, d, J=5 Hz), 6.46 (1H, d, J=10 Hz), 6.60 (1H, d, J=15.8 Hz), 6.82 (1H, dt, J=15.8, 7 Hz), 7.05-7.12 (2H, m), 7.16-7.48 (15H, m), 7.56-7.70 (4H, m);

MASS (ES−): m/e 839.43.

### EXAMPLE 279

Compound E279 was obtained from the Compound E278 in a manner similar to Example 3.

¹H-NMR (300 MHz, CDCl₃, δ): 1.10 (3×3H, s), 1.14-1.29 (4H, m), 1.19 (3H, d, J=7 Hz), 1.37-1.63 (6H, m), 1.67-1.82 (2H, m), 2.50 (2H, m), 3.02 (2H, m), 3.08 (1H, dd, J=13.5, 7 Hz), 3.21 (1H, dd, J=13.5, 4.5 Hz), 3.26 (1H, dd, J=13.5, 8 Hz), 3.64 (1H, dd, J=13.5, 10.5 Hz), 3.72 (1H, ddd, J=8, 5, 4.5 Hz), 3.95 (1H, m), 4.16 (1H, m), 4.19 (1H, q, J=7 Hz), 5.01 (1H, m), 5.36 (1H, m), 6.39 (1H, d, J=5 Hz), 6.42 (1H, d, J=10.5 Hz), 7.07-7.13 (2H, m), 7.16-7.50 (15H, m), 7.58-7.69 (4H, m);

MASS (ES+): m/e 843.41.

### EXAMPLE 280

Compound E280 was obtained from the Compound E279 in a manner similar to Example 6.

¹H-NMR (300 MHz, CDCl₃, δ): 1.12-1.42 (6H, m), 1.38 (3H, d, J=7 Hz), 1.44-1.85 (6H, m), 1.97 (1H, m), 2.08 (1H, m), 2.32-2.56 (2H, m), 3.01 (1H, m), 3.08 (1H, dd, J=14, 7 Hz), 3.21 (1H, dd, J=13, 6 Hz), 3.25 (1H, dd, J=14, 8 Hz), 3.58 (1H, d, J=5 Hz), 3.64 (1H, dd, J=13, 11 Hz), 3.73 (1H, ddd, J=11, 6, 6 Hz), 3.95 (1H, m), 4.10-4.28 (2H, m), 5.02 (1H, m), 5.36 (1H, ddd, J=10.5, 8, 7 Hz), 6.47 (1H, d, J=10 Hz), 6.53 (1H, d, J=6 Hz), 7.07-7.15 (2H, m), 7.17-7.35 (8H, m), 7.45 (1H, d, J=10.5 Hz);

MASS (ES+): m/e 605.28.

### EXAMPLE 281

Compound E281 was obtained from the Compound (507) in a manner similar to Example 1.

¹H-NMR (300 MHz, CDCl₃, δ): 1.10 (3×3H, s), 1.16-1.28 (2H, m), 1.24 (3H, d, J=7 Hz), 1.30-1.44 (2H, m), 1.70-1.91 (2H, m), 2.10-2.36 (4H, m), 3.03 (1H, dd, J=13.5, 6.3 Hz), 3.16-3.38 (3H, m), 3.60-3.80 (2H, m), 3.87 (1H, m), 4.16 (1H, m), 4.27 (1H, m), 4.67 (1H, m), 5.17 (1H, m), 6.37 (1H, d, J=5.5 Hz), 6.60 (1H, d, J=15.7 Hz), 6.84 (1H, dt, J=15.7, 7 Hz), 7.09-7.54 (18H, m), 7.56-7.74 (4H, m);

MASS (ES+): m/e 828.12.

### EXAMPLE 282

Compound E282 was obtained from the Compound E281 in a manner similar to Example 3.

¹H-NMR (300 MHz, CDCl₃, δ): 1.10 (3×3H, s), 1.15-1.28 (4H, m), 1.19 (3H, d, J=6.5 Hz), 1.38-1.64 (3H, m), 1.69-1.86 (3H, m), 2.13-2.36 (2H, m), 2.50 (2H, m), 3.02 (1H, dd, J=13.5, 6.3 Hz), 3.18-3.35 (3H, m), 3.62-3.79 (2H, m), 3.86 (1H, m), 4.13 (1H, m), 4.19 (1H, q, J=6.5 Hz), 4.68 (1H, m), 5.16 (1H, m), 6.33 (1H, d, J=6 Hz), 7.07 (1H, d, J=10 Hz), 7.10-7.16 (2H, m), 7.19-7.49 (14H, m), 7.53 (1H, d, J=10 Hz), 7.58-7.70 (4H, m);

MASS (ES+): m/e 829.77.

### EXAMPLE 283

Compound E283 was obtained from the Compound E282 in a manner similar to Example 6.

¹H-NMR (300 MHz, CDCl₃, δ): 1.14-1.41 (4H, m), 1.38 (3H, d, J=7 Hz), 1.47-1.89 (6H, m), 2.06-2.56 (4H, m), 3.02 (1H, dd, J=13.5, 6.3 Hz), 3.22 (1H, m), 3.27 (1H, dd, J=13.5, 9.5 Hz), 3.31 (1H, dd, J=13, 6 Hz), 3.59 (1H, d, J=4.8 Hz), 3.67 (1H, dd, J=13, 10.5 Hz), 3.74 (1H, ddd, J=10.5, 6, 5.5 Hz), 3.86 (1H, m), 4.16 (1H, m), 4.24 (1H, dq, J=7, 4.8 Hz), 4.68 (1H, m), 5.16 (1H, ddd, J=10.3, 9.5, 6.3 Hz), 6.49 (1H, d, J=5.5 Hz), 7.08-7.34 (11H, m), 7.53 (1H, d, J=10.3 Hz);

MASS (ES+): m/e 591.37.

### EXAMPLE 284

Compound E284 was obtained from the Compound (507) in a manner similar to Example 1.

¹H-NMR (300 MHz, CDCl₃, δ): 1.10 (3×3H, s), 1.34-1.45 (2H, m), 1.52-1.91 (4H, m), 2.12-2.34 (4H, m), 3.00 (1H, dd, J=13.5, 6 Hz), 3.19-3.36 (3H, m), 3.59-3.79 (2H, m), 3.86 (1H, m), 4.16 (1H, m), 4.33 (2H, s), 4.67 (1H, m), 5.16 (1H, m), 6.34 (1H, d, J=5.8 Hz), 6.41 (1H, d, J=15.8 Hz), 6.86 (1H, dt, J=15.8, 7 Hz), 7.09-7.54 (18H, m), 7.62-7.74 (4H, m);

MASS (ES+): m/e 813.66.

### EXAMPLE 285

Compound E285 was obtained from the Compound E284 in a manner similar to Example 3.

¹H-NMR (300 MHz, CDCl₃, δ): 1.10 (3×3H, s), 1.16-1.32 (4H, m), 1.46-1.88 (6H, m), 2.11-2.38 (2H, m), 2.47 (3H, t, J=7.3 Hz), 3.02 (1H, dd, J=14, 6 Hz), 3.20-3.35 (3H, m), 3.62-3.80 (2H, m), 3.86 (1H, m), 4.14 (1H, m), 4.17 (2H, s), 4.67 (1H, m), 5.14 (1H, m), 6.36 (1H, d, J=5.5 Hz), 7.08 (1H, d, J=10.5 Hz), 7.08-7.49 (16H, m), 7.54 (1H, d, J=10.5 Hz), 7.61-7.71 (4H, m);

MASS (ES+): m/e 815.51.

### EXAMPLE 286

Compound E286 was obtained from the Compound E285 in a manner similar to Example 6.

¹H-NMR (300 MHz, CDCl₃, δ): 1.16-1.38 (4H, m), 1.48-1.90 (6H, m), 2.10-2.43 (4H, m), 3.02 (1H, dd, J=13.5, 6 Hz), 3.15 (1H, t, J=4 Hz), 3.22 (1H, m), 3.27 (1H, dd, J=13.5, 9.5 Hz), 3.31 (1H, dd, J=12.5, 5.5 Hz), 3.67 (1H, dd, J=12.5, 10 Hz), 3.74 (1H, ddd, J=10, 5.5, 5 Hz), 3.86 (1H, m), 4.15 (1H, m), 4.24 (2H, d, J=4 Hz), 4.68 (1H, m), 5.16 (1H, ddd, J=10.5, 9.5, 6 Hz), 6.44 (1H, d, J=5 Hz), 7.06-7.35 (11H, m), 7.52 (1H, d, J=10.5 Hz);

MASS (ES+): m/e 577.38.

### EXAMPLE 287

Compound E287 was obtained from the Compound (517) in a manner similar to Example 1.

¹H-NMR (300 MHz, CDCl₃, δ): 1.09 (3×3H, s), 1.16-1.85 (4H, m), 1.23 (3H, d, J=7 Hz), 1.38 (3H, t, J=7 Hz), 1.90-2.26 (4H, m), 2.92-3.30 (4H, m), 3.53 (1H, dd, J=14, 10 Hz), 3.68 (1H, m), 3.95 (1H, m), 3.96 (2H, q, J=7 Hz), 4.18 (1H, m), 4.28 (1H, q, J=7 Hz), 5.01 (1H, m), 5.36 (1H, m), 6.46 (1H, d, J=10 Hz), 6.47 (1H, d, J=5 Hz), 6.61 (1H, d, J=16 Hz), 6.74 (2×1H, d, J=8.5 Hz), 6.83 (1H, dt, J=16, 7 Hz), 6.97 (2×1H, d, J=8.5 Hz), 7.19-7.48 (12H, m), 7.55-7.70 (4H, m);

MASS (ES+): m/e 885.32.

### EXAMPLE 288

Compound E288 was obtained from the Compound E287 in a manner similar to Example 3.

¹H-NMR (300 MHz, CDCl₃, δ): 1.10 (3×3H, s), 1.14-1.24 (4H, m), 1.19 (3H, d, J=7 Hz), 1.34-1.63 (6H, m), 1.38 (3H, t, J=7 Hz), 1.67-1.80 (2H, m), 1.97 (1H, m), 2.10 (1H, m), 2.51 (2H, m), 3.02 (1H, m), 3.08 (1H, dd, J=14, 7.5 Hz), 3.16 (1H, dd, J=13.5, 6.5 Hz), 3.25 (1H, dd, J=14, 8 Hz), 3.56 (1H, dd, J=13.5, 6.5 Hz), 3.67 (1H, ddd, J=10.5, 7, 6.5 Hz), 3.95 (1H, m), 3.98 (2H, q, J=7 Hz), 4.16 (1H, m), 4.19 (1H, q, J=7 Hz), 5.02 (1H, m), 5.35 (1H, m), 6.39 (1H, d, J=7 Hz), 6.43 (1H, d, J=10.5 Hz), 6.76 (2×1H, d, J=8.5 Hz), 6.99 (2×1H, d, J=8.5 Hz), 7.20-7.50 (12H, m), 7.59-7.68 (4H, m);

MASS (ES+): m/e 887.32.

### EXAMPLE 289

Compound E289 was obtained from the Compound E288 in a manner similar to Example 6.

¹H-NMR (300 MHz, CDCl₃, δ): 1.14-1.42 (4H, m), 1.38 (3H, d, J=7 Hz), 1.39 (3H, t, J=7 Hz), 1.44-1.84 (8H, m), 1.90-2.18 (2H, m), 2.45 (2H, m), 3.01 (1H, m), 3.07 (1H, dd, J=14, 7.5 Hz), 3.15 (1H, dd, J=13.5, 6 Hz), 3.25 (1H, dd, J=14, 8 Hz), 3.56 (1H, m), 3.57 (1H, d, J=4.5 Hz), 3.68 (1H, m), 3.95 (1H, m), 3.98 (2H, q, J=7 Hz), 4.16 (1H, m), 4.22 (1H, dq, J=7, 4.5 Hz), 5.01 (1H, m), 5.35 (1H, ddd, J=1.0, 8, 7.5 Hz), 6.42 (1H, d, J=6.5 Hz), 6.45 (1H, d, J=10 Hz);

MASS (ES+): m/e 649.28.

### EXAMPLE 290

Compound E290 was obtained from the Compound (529) in a manner similar to Example 1.

¹H-NMR (300 MHz, CDCl₃, δ): 1.09 (3×3H, s), 1.16-1.86 (8H, m), 1.23 (3H, d, J=7 Hz), 1.38 (3H, t, J=7 Hz), 1.40 (3H, t, J=7 Hz), 1.91-2.25 (4H, m), 2.92-3.06 (2H, m), 3.12-3.25 (2H, m), 3.54 (1H, dd, J=14, 10 Hz), 3.68 (1H, m), 3.97 (1H, m), 4.18 (1H, m), 4.28 (1H, q, J=7 Hz), 5.01 (1H, m), 5.30 (1H, m), 6.34 (1H, d, J=6 Hz), 6.45 (1H, d, J=10 Hz), 6.62 (1H, d, J=15.7 Hz), 6.75 (2×1H, d, J=8.5 Hz), 6.82 (2×1H, d, J=8.5 Hz), 6.83 (1H, m), 6.98 (2×1H, d, J=8.5 Hz), 7.15 (2×1H, d, J=8.5 Hz), 7.30-7.50 (7H, m), 7.56-7.74 (4H, m);

MASS (ES+): m/e 929.47.

### EXAMPLE 291

Compound E291 was obtained from the Compound E290 in a manner similar to Example 3.

¹H-NMR (300 MHz, CDCl₃, δ): 1.10 (3×3H, s), 1.14-1.64 (10H, m), 1.19 (3H, d, J=7 Hz), 1.38 (3H, t, J=7 Hz), 1.40 (3H, t, J=7 Hz), 1.66-1.81 (2H, m), 1.92-2.15 (2H, m), 2.50 (2H, m), 2.94-3.07 (2H, m), 3.12-3.23 (2H, m), 3.55 (1H, dd, J=13, 10.5 Hz), 3.67 (1H, m), 3.94 (1H, m), 3.98 (2H, q, J=7 Hz), 4.00 (2H, q, J=7 Hz), 4.15 (1H, m), 4.19 (1H, q, J=7 Hz), 5.01 (1H, m), 5.30 (1H, m), 6.32 (1H, d, J=6 Hz), 6.41 (1H, d, J=10 Hz), 6.76 (2×1H, d, J=8.5 Hz), 6.82 (2×1H, d, J=8.5 Hz), 6.99 (2×1H, d, J=8.5 Hz), 7.15 (2×1H, d, J=8.5 Hz), 7.32-7.48 (7H, m), 7.58-7.69 (4H, m);

MASS (ES+): m/e 931.60.

### EXAMPLE 292

Compound E292 was obtained from the Compound E291 in a manner similar to Example 6.

¹H-NMR (300 MHz, CDCl₃, δ): 1.14-1.65 (10H, m), 1.38 (3H, d, J=7 Hz), 1.39 (3H, t, J=7 Hz), 1.40 (3H, t, J=7 Hz), 1.67-1.84 (2H, m), 1.91-2.14 (2H, m), 2.33-2.54 (2H, m), 2.92-3.06 (2H, m), 3.10-3.23 (2H, m), 3.56 (1H, dd, J=12, 10.5 Hz), 3.57 (1H, d, J=5 Hz), 3.67 (1H, m), 3.94 (1H, m), 3.98 (2H, q, J=7 Hz), 4.00 (2H, q, J=7 Hz), 4.16 (1H, m), 4.22 (1H, dq, J=7, 5 Hz), 5.01 (1H, m), 5.30 (1H, m), 6.33 (1H, d, J=6 Hz), 6.44 (1H, d, J=10.5 Hz), 6.76 (2×1H, d, J=8.7 Hz), 6.82 (2×1H, d, J=8.7 Hz), 6.99 (2×1H, d, J=8.7 Hz), 7.15 (2×1H, d, J=8.7 Hz), 7.37 (1H, d, J=10 Hz);

MASS (ES+): m/e 693.48.

### EXAMPLE 293

Compound E293 was obtained from the Compound (527) in a manner similar to Example 1.

¹H-NMR (300 MHz, CDCl₃, δ): 1.10 (3×3H, s), 1.20-2.26 (12H, m), 1.37 (3H, t, J=7 Hz), 1.40 (3H, t, J=7 Hz), 2.93-3.06 (2H, m), 3.12-3.24 (2H, m), 3.53 (1H, dd, J=13.5, 10.5 Hz), 3.67 (1H, m), 3.95 (1H, m), 3.96 (2H, q, J=7 Hz), 4.00 (2H, q, J=7 Hz), 4.16 (1H, m), 4.33 (2H, s), 5.00 (1H, m), 5.30 (1H, m), 6.33 (1H, d, J=6 Hz), 6.44 (1H, d, J=16 Hz), 6.45 (1H, d, J=10 Hz), 6.74 (2×1H, d, J=8.7 Hz), 6.82 (2×1H, d, J=8.7 Hz), 6.85 (1H, dt, J=16, 7 Hz), 6.97 (2×1H, d, J=8.7 Hz), 7.15 (2×1H, d, J=8.7 Hz), 7.32-7.48 (7H, m), 7.61-7.73 (4H, m);

MASS (ES+): m/e 915.52.

### EXAMPLE 294

Compound E294 was obtained from the Compound E293 in a manner similar to Example 3.

¹H-NMR (300 MHz, CDCl₃, δ): 1.10 (3×3H, s), 1.15-1.30 (4H, m), 1.38 (3H, t, J=7 Hz), 1.40 (3H, t, J=7 Hz), 1.44-1.65 (6H, m), 1.67-1.80 (2H, m), 1.92-2.14 (2H, m), 2.47 (2H, t, J=7.3 Hz), 2.93-3.07 (2H, m), 3.11-3.23 (2H, m), 3.55 (1H, dd, J=13.5, 10.8 Hz), 3.66 (1H, m), 3.94 (1H, m), 3.97 (2H, q, J=7 Hz), 4.00 (2H, q, J=7 Hz), 4.15 (1H, m), 4.17 (2H, s), 5.01 (1H, m), 5.29 (1H, m), 6.32 (1H, d, J=5.8 Hz), 6.42 (1H, d, J=10.5 Hz), 6.76 (2×1H, d, J=8.8 Hz), 6.82 (2×1H, d, J=8.8 Hz), 6.99 (2×1H, d, J=8.8 Hz), 7.15 (2×1H, d, J=8.8 Hz), 7.34-7.49 (7H, m), 7.61-7.69 (4H, m);

MASS (ES+): m/e 917.56.

### EXAMPLE 295

Compound E295 was obtained from the Compound E294 in a manner similar to Example 6.

¹H-NMR (300 MHz, CDCl₃, δ): 1.06-2.30 (14H, m), 1.39 (3H, t, J=7 Hz), 1.40 (3H, t, J=7 Hz), 2.38 (2H, t, J=7 Hz), 2.90-3.07 (2H, m), 3.09-3.28 (2H, m), 3.54 (1H, dd, J=13, 10 Hz), 3.69 (1H, m), 3.94 (1H, m), 3.98 (2H, q, J=7 Hz), 4.00 (2H, q, J=7 Hz), 4.18 (1H, m), 4.23 (2H, s), 5.02 (1H, m), 5.29 (1H, m), 6.42-6.52 (1H, m), 6.76 (2×1H, d, J=8.5 Hz), 6.82 (2×1H, d, J=8.5 Hz), 7.00 (2×1H, d, J=8.5 Hz), 7.15 (2×1H, d, J=8.5 Hz), 7.39 (2×1H, d, J=10 Hz);

MASS (ES+): m/e 679.40.

### EXAMPLE 296

Compound E296 was obtained from the Compound (527) in a manner similar to Example 1.

¹H-NMR (300 MHz, CDCl₃, δ): 0.80 (3H, t, J=7.5 Hz), 1.11 (3×3H, S), 1.20-1.82 (10H, m), 1.38 (3H, t, J=7 Hz), 1.40 (3H, t, J=7 Hz), 1.91-2.22 (4H, m), 2.94-3.06 (2H, m), 3.13-3.24 (2H, m), 3.53 (1H, dd, J=13.5, 10.5 Hz), 3.68 (1H, m), 3.95 (1H, m), 3.97 (2H, q, J=7 Hz), 4.00 (2H, q, J=7 Hz), 4.10-4.22 (2H, m), 5.01 (1H, m), 5.30 (1H, m), 6.33 (1H, d, J=6 Hz), 6.44 (1H, d, J=10.5 Hz), 6.55 (1H, d, J=16 Hz), 6.74 (1H, m), 6.75 (2×1H, d, J=8.5 Hz), 6.82 (2×1H, d, J=8.5 Hz), 6.98 (2×1H, d, J=8.5 Hz), 7.15 (2×1H, d, J=8.5 Hz), 7.29-7.48 (7H, m), 7.56-7.68 (4H, m);

MASS (ES+): m/e 943.60.

### EXAMPLE 297

Compound E297 was obtained from the Compound E296 in a manner similar to Example 3.

¹H-NMR (300 MHz, CDCl₃, δ): 0.81 (3H, t, J=7.5 Hz), 1.05-1.78 (14H, m), 1.11 (3×3H, s), 1.38 (3H, t, J=7 Hz), 1.40 (3H, t, J=7 Hz), 1.90-2.14 (2H, m), 2.38 (2H, m), 2.94-3.07 (2H, m), 3.11-3.23 (2H, m), 3.55 (1H, dd, J=13, 10 Hz), 3.66 (1H, m), 3.93 (1H, m), 3.97 (2H, q, J=7 Hz), 4.00 (2H, q, J=7 Hz), 4.06-4.18 (2H, m), 5.01 (1H, m), 5.29 (1H, m), 6.28 (1H, d, J=5.5 Hz), 6.40 (1H, d, J=10 Hz), 6.76 (2×1H, d, J=8.5 Hz), 6.82 (2×1H, d, J=8.5 Hz), 6.98 (2×1H, d, J=8.5 Hz), 7.15 (2×1H, d, J=8.5 Hz), 7.31-7.47 (7H, m), 7.57-7.67 (4H, m);

MASS (ES+): m/e 945.55.

### EXAMPLE 298

Compound E298 was obtained from the Compound E297 in a manner similar to Example 6.

¹H-NMR (300 MHz, CDCl₃, δ): 0.94 (3H, t, J=7.3 Hz), 1.14-1.43 (6H, m), 1.39 (3H, t, J=7 Hz), 1.40 (3H, t, J=7 Hz), 1.46-2.16 (8H, m), 2.43 (2H, m), 2.92-3.06 (2H, m), 3.16 (1H, dd, J=13, 6 Hz), 3.18 (1H, dd, J=13.5, 7.5 Hz), 3.52 (1H, d, J=5 Hz), 3.55 (1H, m), 3.67 (1H, m), 3.94 (1H, m), 3.98 (2H, q, J=7 Hz), 4.00 (2H, q, J=7 Hz), 4.10-4.22 (2H, m), 5.01 (1H, m), 5.29 (1H, m), 6.40 (1H, d, J=5.5 Hz), 6.45 (1H, d, J=10 Hz), 6.76 (2×1H, d, J=8.7 Hz), 6.81 (2×1H, d, J=8.7 Hz), 6.99 (2×1H, d, J=8.7 Hz), 7.15 (2×1H, d, J=8.7 Hz), 7.38 (1H, d, J=10.5 Hz);

MASS (ES+): m/e 707.42.

### EXAMPLE 299

Compound E299 was obtained from the Compound (537) in a manner similar to Example 1.

¹H-NMR (300 MHz, CDCl₃, δ): 1.09 (3×3H, s), 1.22 (3H, d, J=6.5 Hz), 1.35-1.50 (2H, m), 1.39 (3H, t, J=7 Hz), 1.58-1.88 (4H, m), 2.11-2.39 (4H, m), 2.76 (1H, dd, J=14, 7 Hz), 2.87 (1H, dd, J=13.5, 5.5 Hz), 3.02-3.24 (3H, m), 3.95 (1H, m), 3.99 (2H, q, J=7 Hz), 4.25 (1H, m), 4.26 (1H, q, J=6.5 Hz), 4.61 (1H, dd, J=8, 2 Hz), 4.68 (1H, m), 5.06 (1H, m), 6.33 (1H, d, J=10 Hz), 6.45 (1H, d, J=10.5 Hz), 6.59 (1H, d, J=16 Hz), 6.80 (2×1H, d, J=8.7 Hz), 6.84 (1H, dt, J=16, 7 Hz), 7.11 (2×1H, d, J=8.7 Hz), 7.15-7.48 (12H, m), 7.56-7.70 (4H, m);

MASS (ES+): m/e 871.38.

### EXAMPLE 300

Compound E300 was obtained from the Compound E299 in a manner similar to Example 3.

¹H-NMR (300 MHz, CDCl₃, δ): 1.10 (3×3H, s), 1.14-1.28 (4H, m), 1.18 (3H, d, J=7 Hz), 1.33-1.50 (2H, m), 1.39 (3H, t, J=7 Hz), 1.54-1.82 (4H, m), 2.20 (1H, m), 2.32 (1H, m), 2.49 (2H, m), 2.76 (1H, dd, J=14, 7 Hz), 2.86 (1H, dd, J=13.5, 5 Hz), 3.02-3.24 (3H, m), 3.94 (1H, m), 3.99 (2H, q, J=7 Hz), 4.18 (1H, q, J=7 Hz), 4.24 (1H, m), 4.61 (1H, dd, J=8, 2.5 Hz), 4.68 (1H, m), 5.06 (1H, m), 6.31 (1H, d, J=10 Hz), 6.47 (1H, d, J=10.5 Hz), 6.80 (2×1H, d, J=9 Hz), 6.98-7.30 (8H, m), 7.32-7.48 (6H, m), 7.58-7.68 (4H, m);

MASS (ES+): m/e 873.47.

### EXAMPLE 301

Compound E301 was obtained from the Compound E300 in a manner similar to Example 6.

¹H-NMR (300 MHz, CDCl₃, δ): 1.19-1.35 (4H, m), 1.38 (3H, d, J=7.3 Hz), 1.40 (3H, d, J=7 Hz), 1.52-1.88 (6H, m), 2.12-2.56 (4H, m), 2.77 (1H, dd, J=14, 7 Hz), 2.87 (1H, dd, J=13, 5 Hz), 3.01-3.24 (3H, m), 3.56 (1H, br), 3.94 (1H, m), 4.00 (2H, q, J=7 Hz), 4.17-4.30 (2H, m), 4.61 (1H, dd, J=8, 3 Hz), 4.68 (1H, m), 5.06 (1H, m), 6.36 (1H, d, J=10 Hz), 6.48 (1H, d, J=10.5 Hz), 6.80 (2×1H, d, J=8.5 Hz), 7.11 (2×1H, d, J=8.5 Hz), 7.13-7.32 (6H, m);

MASS (ES+): m/e 635.

### EXAMPLE 302

Compound E302 was obtained from the Compound (545) in a manner similar to Example 1.

¹H-NMR (300 MHz, CDCl₃, δ): 1.09 (3×3H, s), 1.22 (3H, d, J=7 Hz), 1.34-1.51 (2H, m), 1.57-1.88 (4H, m), 2.10-2.39 (4H, m), 2.84 (1H, dd, J=14, 7 Hz), 2.88 (1H, dd, J=13, 5 Hz), 3.08 (1H, m), 3.19 (1H, dd, J=13, 10.5 Hz), 3.22 (1H, dd, J=14, 8 Hz), 3.94 (1H, m), 4.26 (1H, m), 4.27 (1H, q, J=7 Hz), 4.61 (1H, dd, J=8, 2.5 Hz), 4.75 (1H, ddd, J=10, 8, 7 Hz), 5.07 (1H, ddd, J=10.5, 10.5, 5 Hz), 6.37 (1H, d, J=10 Hz), 6.48 (1H, d, J=10.5 Hz), 6.58 (1H, d, J=16 Hz), 6.84 (1H, dt, J=16, 7 Hz), 7.14-7.48 (17H, m), 7.55-7.69 (4H, m);

MASS (ES+): m/e 827.56.

### EXAMPLE 303

Compound E303 was obtained from the Compound E302 in a manner similar to Example 3.

¹H-NMR (300 MHz, CDCl₃, δ): 1.10 (3×3H, s), 1.15-1.30 (4H, m), 1.18 (3H, d, J=7 Hz), 1.36-1.50 (2H, m), 1.52-1.84 (4H, m), 2.12-2.40 (2H, m), 2.48 (2H, m), 2.84 (1H, dd, J=14, 7 Hz), 2.87 (1H, dd, J=13.5, 5 Hz), 3.09 (1H, m), 3.19 (1H, dd, J=13.5, 10 Hz), 3.22 (1H, dd, J=14, 8 Hz), 3.94 (1H, m), 4.18 (1H, q, J=7 Hz), 4.24 (1H, m), 4.61 (1H, dd, J=8, 2 Hz), 4.74 (1H, ddd, J=10, 8, 7 Hz), 5.06 (1H, ddd, J=10.5, 10, 5 Hz), 6.35 (1H, d, J=10 Hz), 6.49 (1H, d, J=10.5 Hz), 7.12 (1H, d, J=10.5 Hz), 7.12-7.32 (10H, m), 7.32-7.48 (6H, m), 7.58-7.68 (4H, m);

MASS (ES+): m/e 829.

### EXAMPLE 304

Compound E304 was obtained from the Compound E303 in a manner similar to Example 6.

¹H-NMR (300 MHz, CDCl₃, δ): 1.18-1.34 (4H, m), 1.38 (3H, d, J=7.3 Hz), 1.52-1.87 (6H, m), 2.12-2.55 (4H, m), 2.85 (1H, dd, J=14, 7.5 Hz), 2.87 (1H, dd, J=13.5, 5 Hz), 3.08 (1H, m), 3.18 (1H, dd, J=13.5, 10.5 Hz), 3.21 (1H, dd, J=14, 8 Hz), 3.56 (1H, d, J=4.8 Hz), 3.94 (1H, m), 4.17-4.30 (2H, m), 4.62 (1H, dd, J=8, 2.5 Hz), 4.74 (1H, ddd, J=10, 8, 7.5 Hz), 5.06 (1H, ddd, J=10.5, 10.5, 5 Hz), 6.40 (1H, d, J=10 Hz), 7.12-7.32 (11H, m);

MASS (ES+): m/e 591.

### EXAMPLE 305

Compound E305 was obtained from the Compound (550) in a manner similar to Example 1.

¹H-NMR (300 MHz, CDCl₃, δ): 0.74 (3H, m), 0.79 (3H, d, J=6 Hz), 1.19-1.97 (12H, m), 1.23 (3H, d, J=7 Hz), 2.23 (2H, m), 2.46 (1H, m), 2.69 (1H, m), 2.88 (1H, dd, J=14, 6 Hz), 3.25 (1H, dd, J=14, 9 Hz), 4.27 (1H, q, J=7 Hz), 4.43-4.70 (4H, m), 4.81 (1H, m), 5.81-5.95 (2H, br), 6.16 (1H, d, J=10 Hz), 6.61 (1H, d, J=16 Hz), 6.85 (1H, dt, J=16, 7 Hz), 7.15-7.29 (5H, m), 7.30-7.48 (6H, m), 7.56-7.69 (4H, m);

MASS (ES+): m/e 807.51.

### EXAMPLE 306

Compound E306 was obtained from the Compound E305 in a manner similar to Example 3.

¹H-NMR (300 MHz, CDCl₃, δ): 0.73 (3H, m), 0.78 (3H, d, J=6.5 Hz), 1.10 (3×3H, s), 1.16-1.94 (16H, m), 1.18 (3H, d, J=6.8 Hz), 2.40-2.53 (3H, m), 2.70 (1H, m), 2.87 (1H, dd, J=13.8, 6.3 Hz), 3.25 (1H, dd, J=13.8, 9.8 Hz), 4.19 (1H, q, J=6.8 Hz), 4.40-4.69 (4H, m), 4.80 (1H, m), 5.82-5.93 (2H, m), 6.17 (1H, d, J=11 Hz), 7.15-7.28 (5H, m), 7.32-7.48 (6H, m), 7.58-7.68 (4H, m);

MASS (ES+): m/e 809.60.

### EXAMPLE 307

Compound E307 was obtained from the Compound E306 in a manner similar to Example 6.

¹H-NMR (300 MHz, CDCl₃, δ): 0.72 (3H, m), 0.78 (3H, d, J=7 Hz), 1.09 (1H, m), 1.18-1.94 (15H, m), 1.38 (3H, d, J=7.3 Hz), 2.34-2.58 (3H, m), 2.72 (1H, m), 2.88 (1H, dd, J=13.5, 6.3 Hz), 3.25 (1H, dd, J=13.5, 9.5 Hz), 3.56 (1H, d, J=4 Hz), 4.23 (1H, m), 4.44-4.70 (4H, m), 4.84 (1H, m), 5.98-6.11 (2H, br), 6.24 (1H, d, J=11 Hz), 7.14-7.29 (5H, m);

MASS (ES+): m/e 571.60.

### EXAMPLE 308

Compound E308 was obtained from the Compound (78) in a manner similar to Example 1.

¹H-NMR (300 MHz, CDCl₃, δ): 0.83 (3H, t, J=7.3 Hz), 1.09 (3×3H, s), 1.22 (3H, d, J=7 Hz), 1.28 (3H, s), 1.38-1.53 (2H, m), 1.61 (1H, m), 1.71-1.91 (3H, m), 2.09-2.40 (6H, m), 2.90 (1H, dd, J=13.5, 5.5 Hz), 3.18 (1H, dd, J=13.5, 9.5 Hz), 3.26 (1H, m), 3.86 (1H, m), 4.21 (1H, m), 4.27 (1H, q, J=7 Hz), 4.67 (1H, m), 5.03 (2H, s), 5.14 (1H, ddd, J=10, 9.5, 5.5 Hz), 5.80 (1H, s), 6.62 (1H, d, J=16 Hz), 6.87 (1H, m), 6.89 (2×1H, d, J=8.8 Hz), 7.13 (1H, d, J=10 Hz), 7.14 (2×1H, d, J=8.8 Hz), 7.30-7.48 (11H, m), 7.50 (1H, d, J=10 Hz), 7.56-7.70 (4H, m);

MASS (ES+): m/e 885.20.

### EXAMPLE 309

Compound E309 was obtained from the Compound E308 in a manner similar to Example 3.

¹H-NMR (300 MHz, CDCl₃, δ): 0.83 (3H, t, J=7.3 Hz), 1.10 (3×3H, s), 1.16-1.36 (4H, m), 1.18 (3H, d, J=7 Hz), 1.28 (3H, s), 1.40-1.52 (2H, m), 1.61 (1H, m), 1.70-1.89 (3H, m), 2.06-2.40 (4H, m), 2.51 (2H, m), 2.89 (1H, dd, J=13.5, 5.5 Hz), 3.18 (1H, dd, J=13.5, 10 Hz), 3.26 (1H, m), 3.85 (1H, m), 4.18 (1H, q, J=7 Hz), 4.18 (1H, m), 4.67 (1H, m), 5.03 (2H, s), 5.13 (1H, ddd, J=10, 10, 5.5 Hz), 5.81 (1H, s), 6.89 (2×1H, d, J=8.5 Hz), 7.08 (1H, d, J=10.3 Hz), 7.15 (2×1H, d, J=8.5 Hz), 7.30-7.49 (11H, m), 7.55 (1H, d, J=10 Hz), 7.59-7.70 (4H, m);

MASS (ES+): m/e 887.31.

### EXAMPLE 310

Compound E310 was obtained from the Compound E309 in a manner similar to Example 6.

¹H-NMR (300 MHz, CDCl₃, δ): 0.84 (3H, t, J=7.5 Hz), 1.24-1.44 (4H, m), 1.28 (3H, s), 1.38 (3H, d, J=7 Hz), 1.54-1.70 (3H, m), 1.71-1.90 (3H, m), 2.07-2.58 (6H, m), 2.89 (1H, dd, J=13.5, 6.5 Hz), 3.18 (1H, dd, J=13.5, 10 Hz), 3.26 (1H, m), 3.55 (1H, d, J=4.5 Hz), 3.86 (1H, m), 4.14-4.28 (2H, m), 4.67 (1H, m), 5.03 (1H, S), 5.13 (1H, ddd, J=10, 10, 6.5 Hz), 5.79 (1H, s), 6.89 (2×1H, d, J=8.5 Hz), 7.11 (1H, d, J=10 Hz), 7.15 (2×1H, d, J=8.5 Hz), 7.25-7.47 (5H, m), 7.52 (1H, d, J=10 Hz);

MASS (ES+): m/e 649.36.

### EXAMPLE 311

The Compound E9 (320 mg) was dissolved in tetrahydrofuran (4 ml). Cold hydrogen fluoride-pyridine (1 ml) was added to the mixture and the mixture was stirred at ambient temperature for about 3 hours. The reaction mixture was neutralized with saturated aqueous sodium hydrogen bicarbonate and then with 1N aqueous sodium hydroxide. The mixture was extracted with ethyl acetate. The ethyl acetate layer was washed with saturated brine and dried over sodium sulfate, and the solvent was removed by evaporation. The residue was purified by thin layer chromatography (eluting with ethyl acetate) and lyophilized from t-butanol to give the objective Compound E311 as a white amorphous.

¹H-NMR (300 MHz, CDCl₃, δ): 0.85 (3H, t, J=7.5 Hz), 1.29 (3H, s), 1.38 (3H, d, J=7 Hz), 1.42-1.92 (6H, m), 2.08-2.41 (6H, m), 2.89 (1H, dd, J=13.5, 6 Hz), 3.18 (1H, dd, J=13.5, 9.5 Hz), 3.26 (1H, m), 3.65 (1H, d, J=5 Hz), 3.77 (1H, s), 3.86 (1H, m), 4.22 (1H, dt, J=10, 7.5 Hz), 4.44 (1H, dq, J=7.5, 5 Hz), 4.67 (1H, m), 5.14 (1H, ddd, J=10, 9.5, 6 Hz), 5.90 (1H, s), 6.25 (1H, brd, J=16 Hz), 6.81 (2×1H, d, J=8.5 Hz), 7.01 (1H, dt, J=16, 7 Hz), 7.14 (2×1H, d, J=8.5 Hz), 7.18 (1H, d, J=10 Hz), 7.48 (1H, d, J=10 Hz);

MASS (ES+): m/e 571.35;

[α]_{D}³⁰=−104.1° (c=0.32, CHCl₃).

### EXAMPLE 312

Compound E312 was obtained from the Compound E26 in a manner similar to Preparation 78.

¹H-NMR (300 MHz, CDCl₃, δ): 0.84 (3H, t, J=7.3 Hz), 1.22-1.42 (4H, m), 1.28 (3H, s), 1.39 (3H, t, J=7 Hz), 1.51-1.89 (6H, m), 2.07-2.38 (4H, m), 2.34 (3H, s), 2.73 (2H, t, J=7.3 Hz), 2.88 (1H, dd, J=13.5, 6 Hz), 3.17 (1H, dd, J=13.5, 10 Hz), 3.25 (1H, m), 3.85 (1H, m), 3.99 (2H, q, J=7 Hz) 4.19 (1H, dt, J=10, 7.5 Hz), 4.66 (1H, m), 5.13 (1H, ddd, J=10, 10, 6 Hz), 5.80 (1H, s), 6.80 (2×1H, d, J=8.5 Hz), 7.09 (1H, d, J=10 Hz), 7.13 (2×1H, d, J=8.5 Hz), 7.52 (1H, d, J=10 Hz);

MASS (ES+): m/e 585.45;

[α]_{D}²²=−111.3° (c=0.23, CHCl₃).

### EXAMPLE 313

Compound E313 was obtained from the Compound E5 in a manner similar to Preparation (387).

¹H-NMR (300 MHz, CDCl₃, δ): 0.84 (3H, t, J=7.2 Hz), 1.10 (3×3H, s), 1.16-1.32 (4H, m), 1.18 (3H, d, J=7 Hz), 1.28 (3H, s), 1.38-1.62 (3H, m), 1.70-1.86 (3H, m), 2.08-2.39 (4H, m), 2.51 (2H, m), 2.89 (1H, dd, J=13.5, 6 Hz), 3.18 (1H, dd, J=13.5, 9.5 Hz), 3.26 (1H, m), 3.85 (1H, m), 4.12-4.24 (2H, m), 4.49 (2H, ddd, J=5, 1.5, 1.5 Hz), 4.67 (1H, m), 5.13 (1H, ddd, J=10.3, 9.5, 6 Hz), 5.27 (1H, ddt, J=10.3, 1.5, 1.5 Hz), 5.40 (1H, ddt, J=17.2, 1.5, 1.5 Hz), 5.83 (1H, s), 6.04 (1H, ddt, J=17.2, 10.3, 5 Hz), 6.82 (2×1H, d, J=8.6 Hz), 7.08 (1H, d, J=10.2 Hz), 7.14 (2×1H, d, J=8.6 Hz), 7.32-7.48 (6H, m), 7.55 (1H, d, J=10.3 Hz), 7.58-7.67 (4H, m);

MASS (ES+): m/e 837.50.

### EXAMPLE 314

Compound E314 was obtained from the Compound E313 in a manner similar to Example 6.

¹H-NMR (300 MHz, CDCl₃, δ): 0.84 (3H, t, J=7.5 Hz), 1.20-1.41 (4H, m), 1.29 (3H, s), 1.38 (3H, d, J=7 Hz), 1.52-1.70 (3H, m), 1.71-1.89 (3H, m), 2.07-2.58 (6H, m), 2.89 (1H, dd, J=13.5, 6 Hz), 3.18 (1H, dd, J=13.5, 9.5 Hz), 3.26 (1H, m), 3.55 (1H, d, J=4.5 Hz), 3.85 (1H, m), 4.13-4.29 (2H, m), 4.50 (2H, d, J=5.5 Hz), 4.67 (1H, dd, J=8, 2 Hz), 5.13 (1H, ddd, J=10, 9.5, 6 Hz), 5.27 (1H, dd, J=10, 1.5 Hz), 5.40 (1H, dd, J=17, 1.5 Hz), 5.84 (1H, s), 6.04 (1H, ddt, J=17, 10, 5.5 Hz), 6.83 (2×1H, d, J=8.5 Hz), 7.11 (1H, d, J=10 Hz), 7.13 (2×1H, d, J=8.5 Hz), 7.52 (1H, d, J=10 Hz);

MASS (ES+): m/e 599.53;

[α]_{D}²⁵=−110.4° (c=0.24, CHCl₃).

The compounds obtained in the above-mentioned Preparations and Examples are listed in the following Tables 2-1 to 2-109.

TABLE 2
Table 2-1
Compound (1)

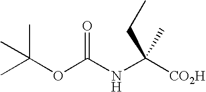

Compound (5)

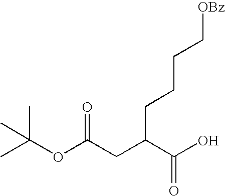

Compound (12)

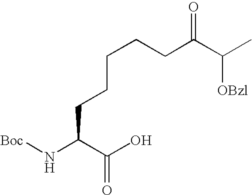

Compound (13)

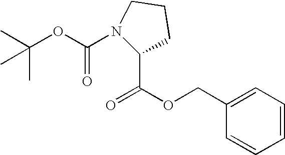

Compound (14)

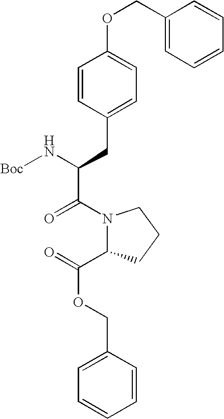

Compound (15)

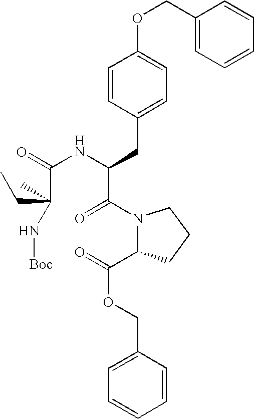

Compound (16)

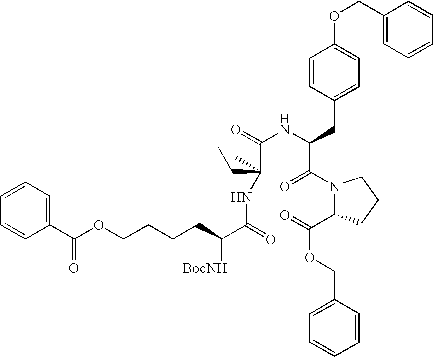

Compound (17)

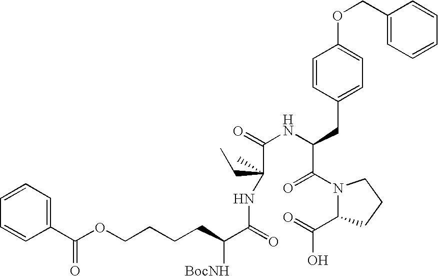

TABLE 2-2
Compound (18)

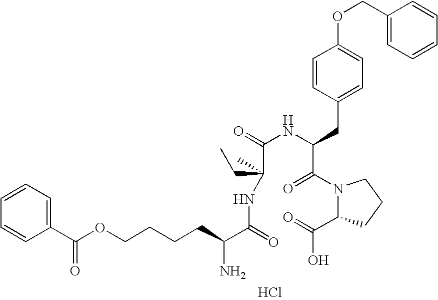

Compound (19)

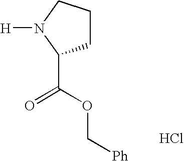

Compound (20)

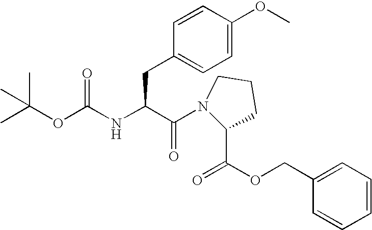

Compound (21)

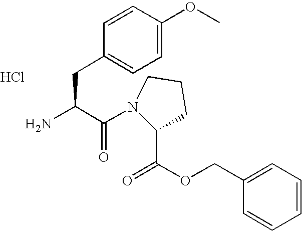

Compound (22)

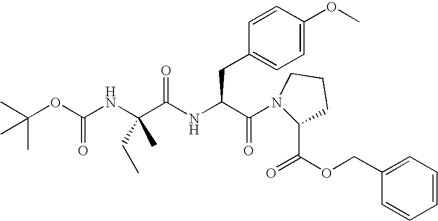

Compound (23)

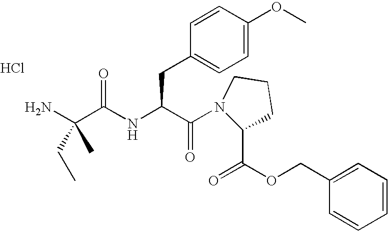

Compound (24)

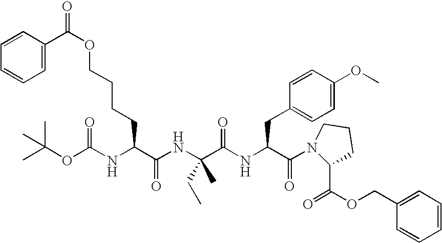

Compound (25)

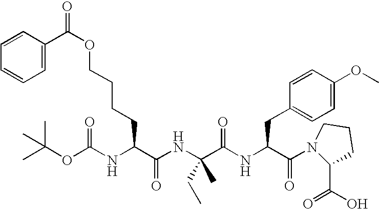

TABLE 2-3
Compound (26)

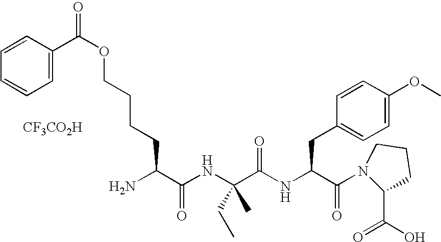

Compound (27)

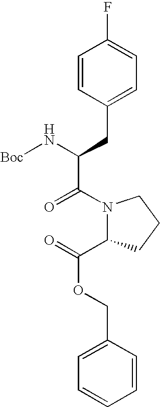

Compound (28)

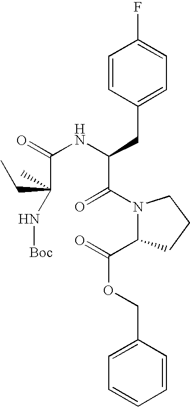

Compound (29)

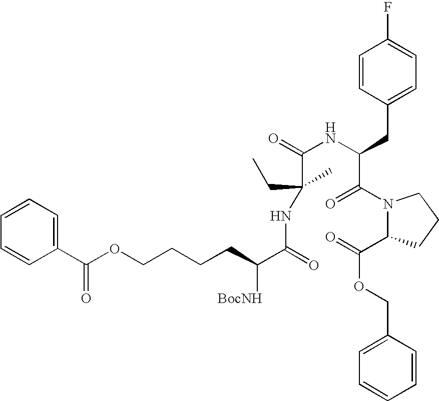

Compound (30)

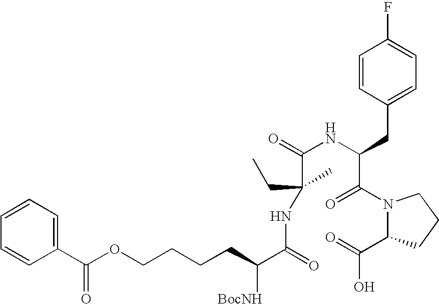

Compound (31)

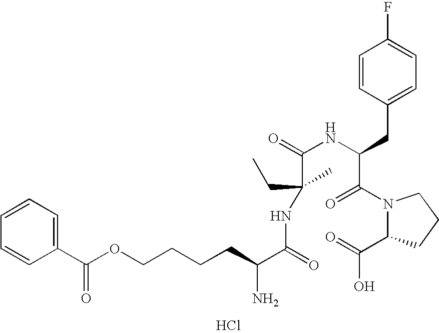

Compound (32)

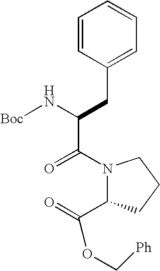

Compound (33)

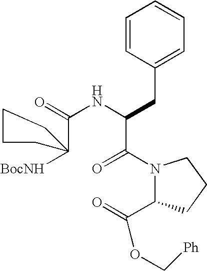

TABLE 2-4
Compound (34)

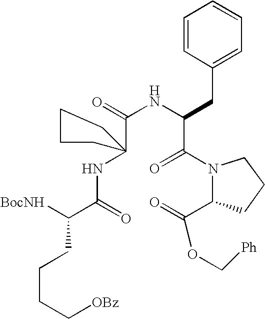

Compound (35)

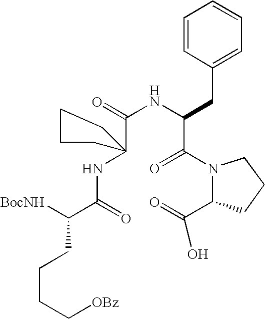

Compound (36)

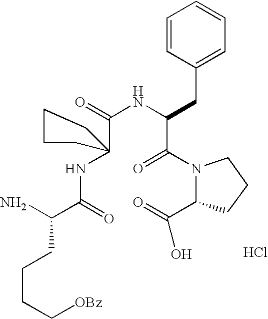

Compound (37)

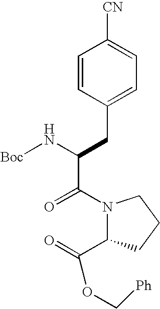

Compound (38)

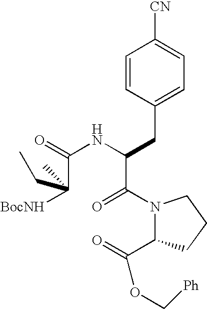

Compound (39)

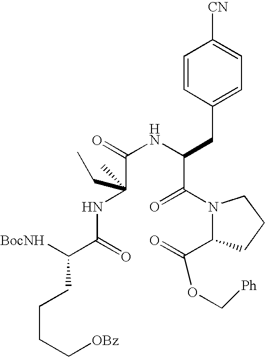

Compound (40)

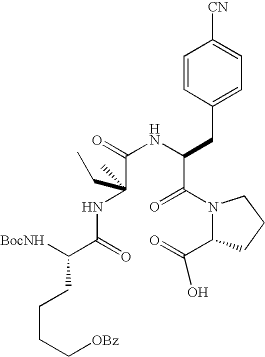

Compound (41)

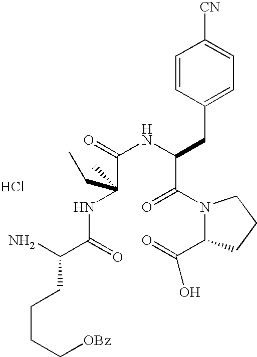

TABLE 2-5
Compound (42)

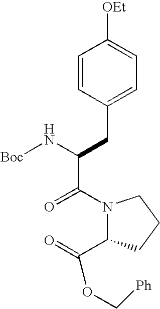

Compound (43)

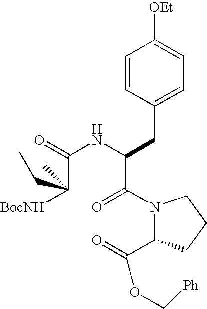

Compound (44)

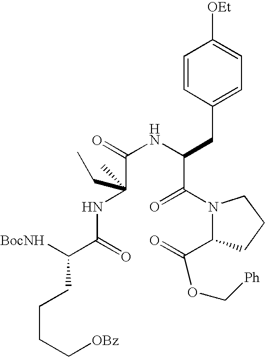

Compound (45)

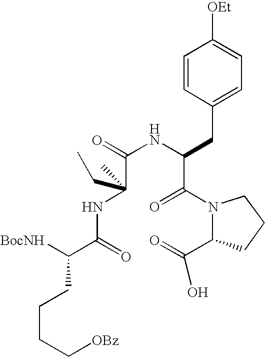

Compound (46)

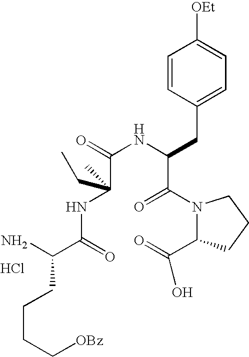

Compound (47)

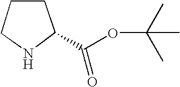

Compound (48)

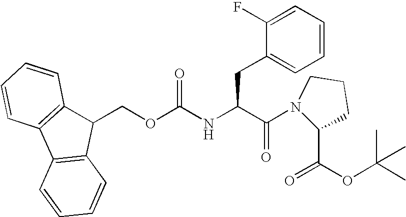

Compound (49)

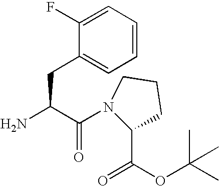

TABLE 2-6
Compound (50)

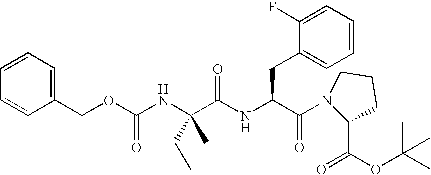

Compound (51)

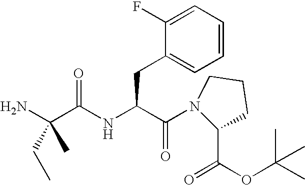

Compound (52)

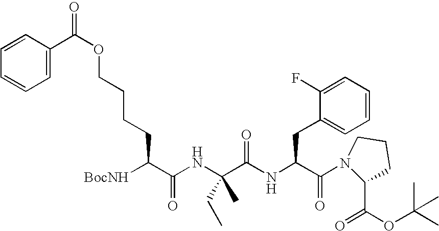

Compound (53)

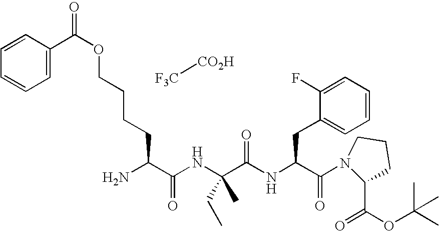

Compound (54)

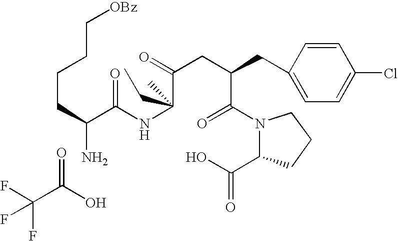

Compound (55)

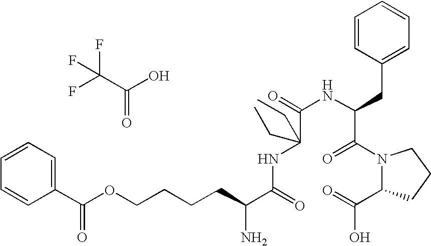

Compound (56)

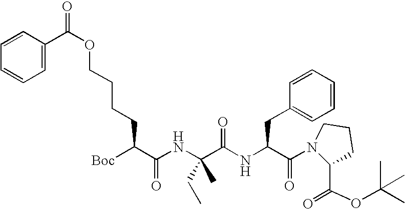

Compound (57)

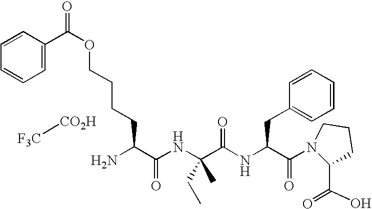

TABLE 2-7
Compound (58)

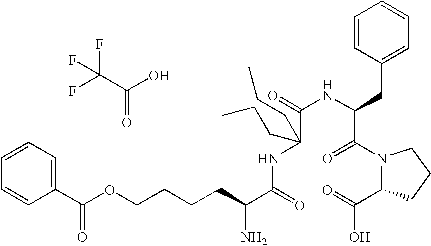

Compound (59)

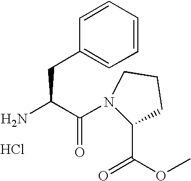

Compound (60)

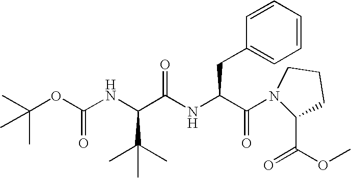

Compound (61)

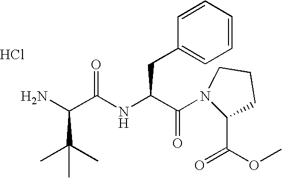

Compound (62)

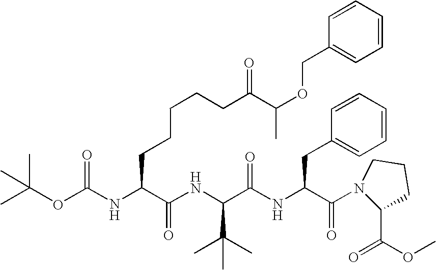

Compound (63)

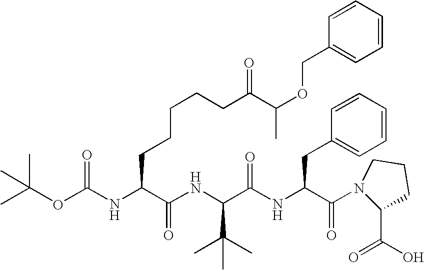

Compound (64)

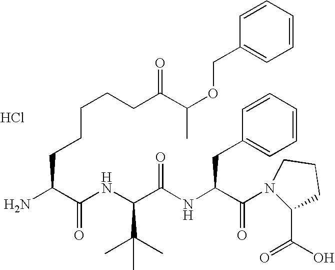

Compound (65)

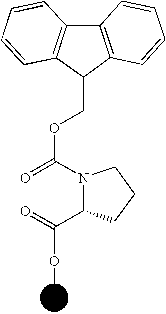

TABLE 2-8
Compound (66)

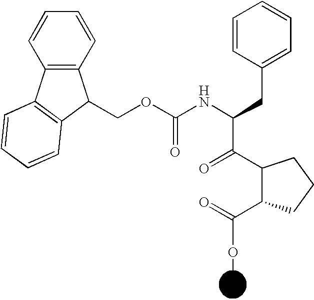

Compound (67)

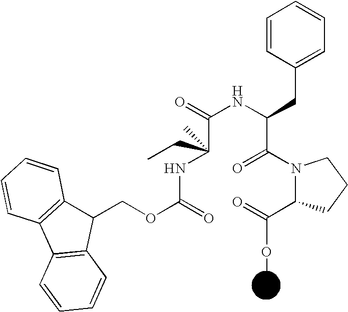

Compound (68)

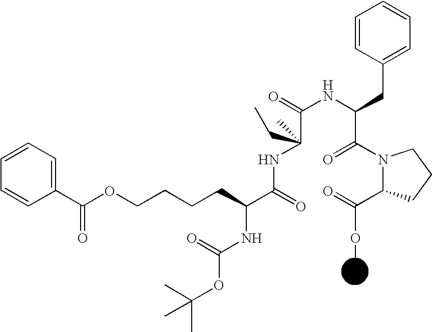

Compound (69)

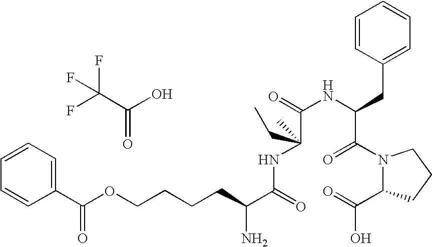

Compound (70)

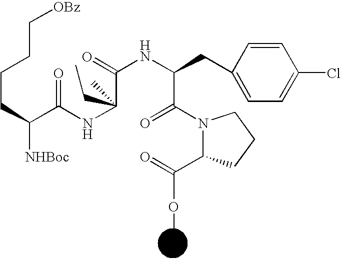

Compound (71)

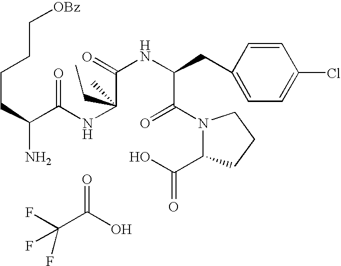

Compound (72)

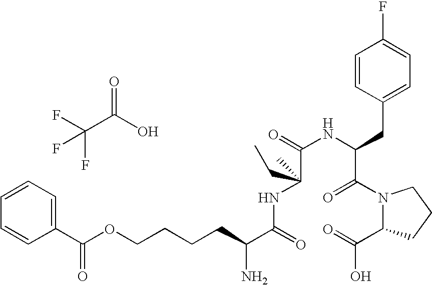

Compound (73)

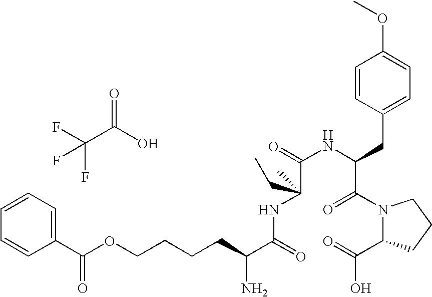

TABLE 2-9
Compound (74)

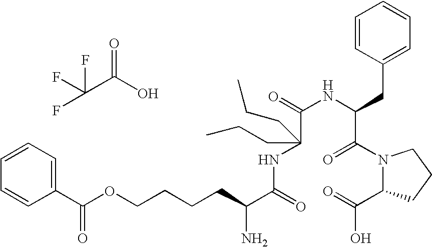

Compound (75)

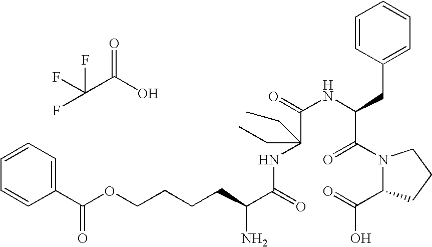

Compound (76)

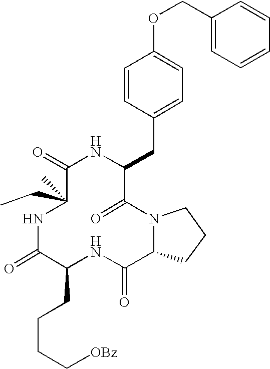

Compound (77)

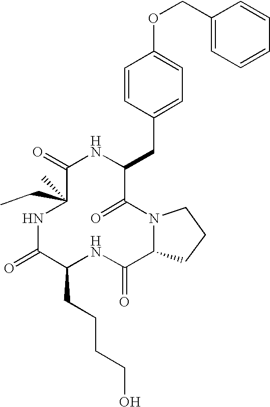

Compound (78)

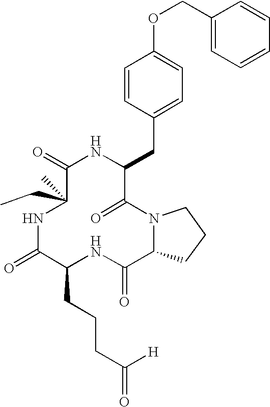

Compound (79)

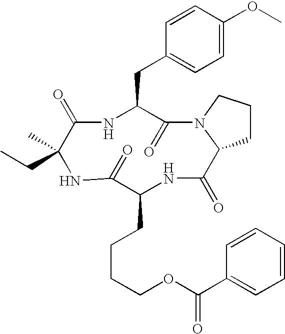

Compound (80)

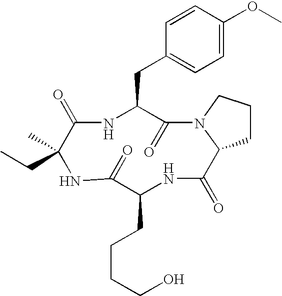

Compound (81)

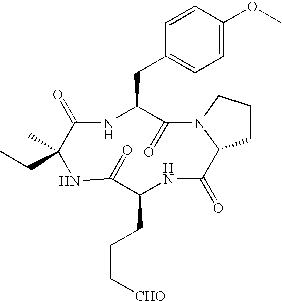

TABLE 2-10
Compound (82)

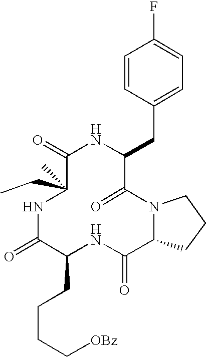

Compound (83)

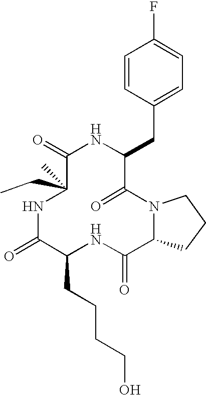

Compound (84)

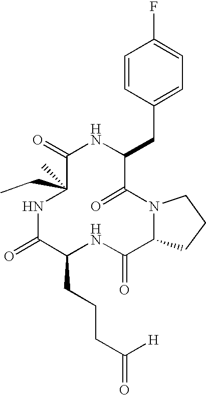

Compound (85)

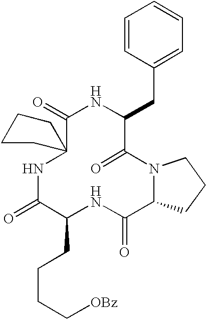

Compound (86)

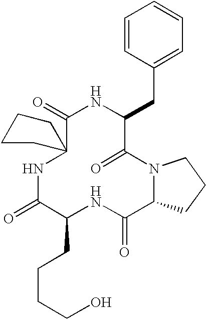

Compound (87)

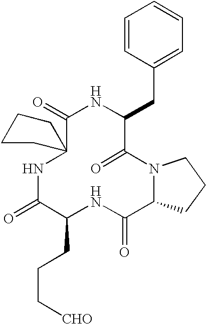

Compound (88)

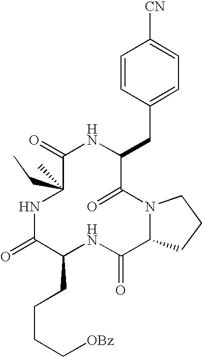

Compound (89)

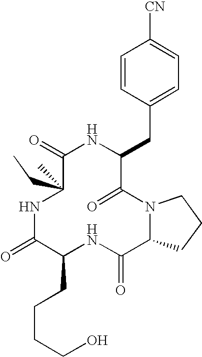

TABLE 2-11
Compound (90)

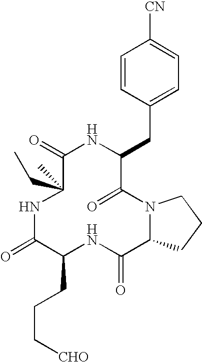

Compound (91)

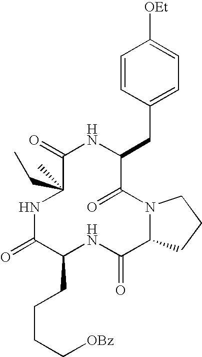

Compound (92)

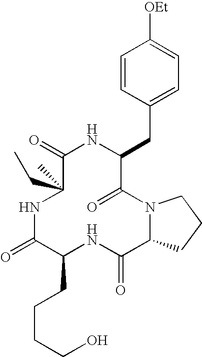

Compound (93)

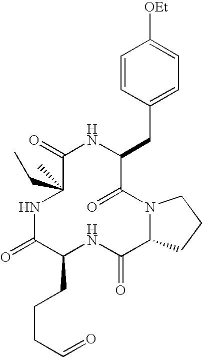

Compound (94)

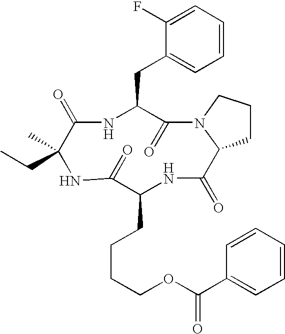

Compound (95)

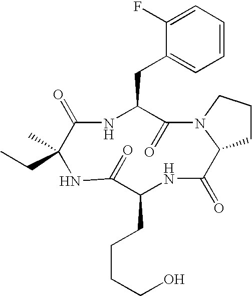

Compound (96)

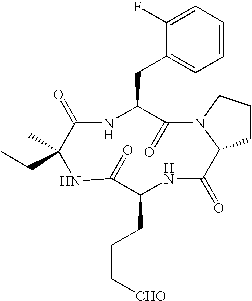

Compound (97)

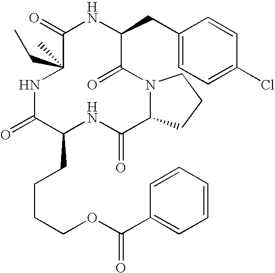

TABLE 2-12
Compound (98)

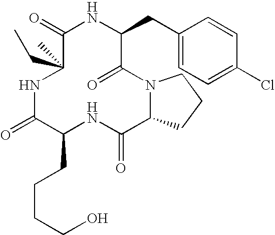

Compound (99)

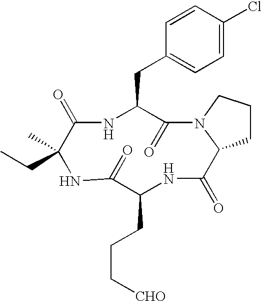

Compound (100)

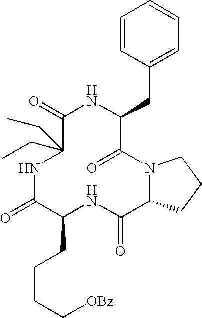

Compound (101)

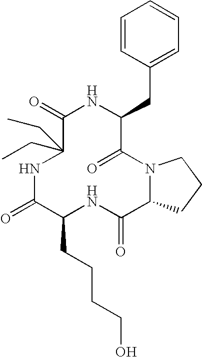

Compound (102)

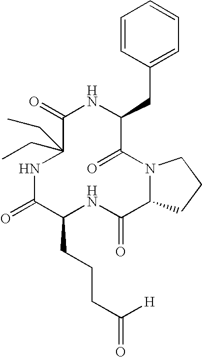

Compound (103)

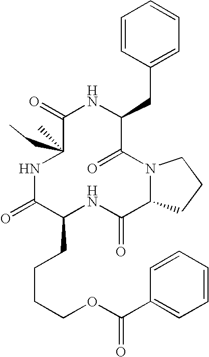

Compound (104)

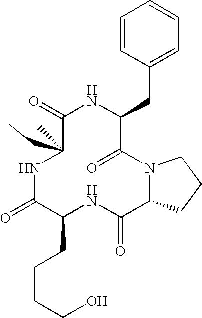

Compound (105)

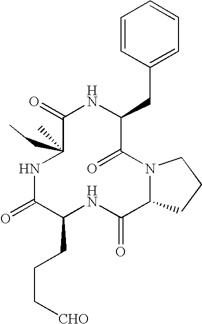

TABLE 2-13
Compound (106)

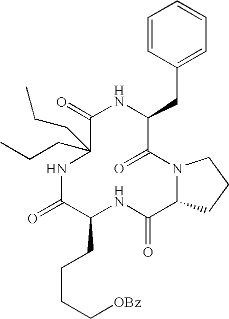

Compound (107)

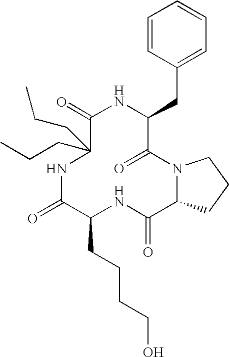

Compound (108)

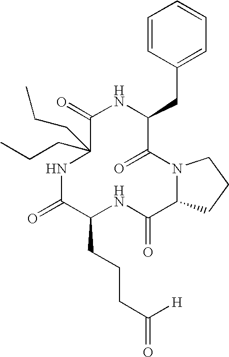

Compound (109)

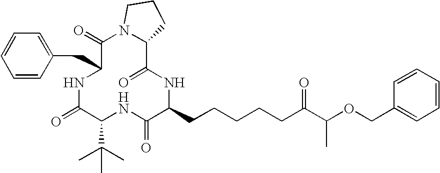

TABLE 2-14
Compound (110)

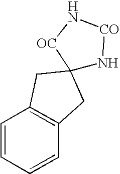

Compound (111)

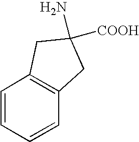

Compound (112)

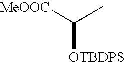

Compound (113)

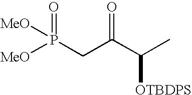

Compound (114)

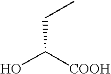

Compound (115)

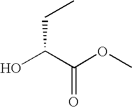

Compound (116)

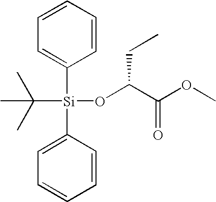

Compound (117)

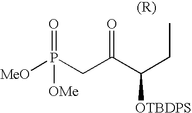

TABLE 2-15
Compound (118)

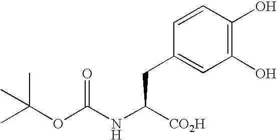

Compound (119)

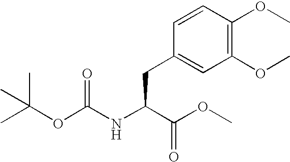

Compound (120)

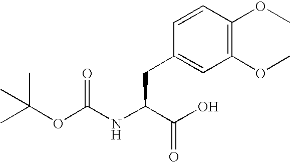

Compound (121)

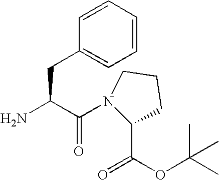

Compound (122)

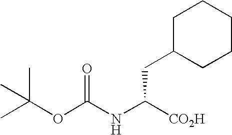

Compound (123)

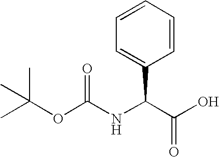

Compound (124)

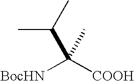

Compound (125)

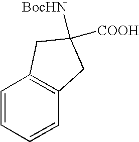

TABLE 2-16
Compound (126)

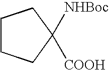

Compound (127)

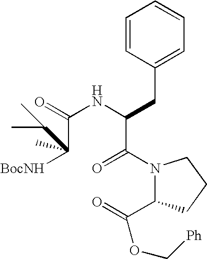

Compound (128)

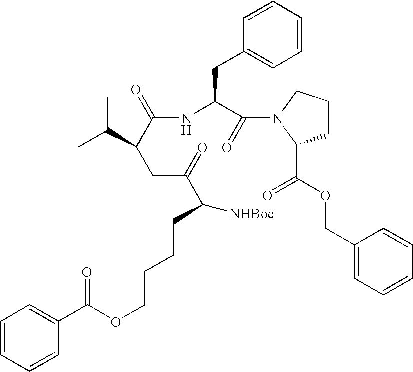

Compound (129)

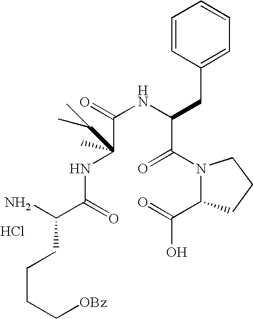

Compound (130)

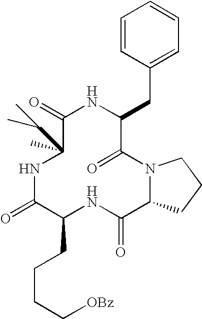

Compound (131)

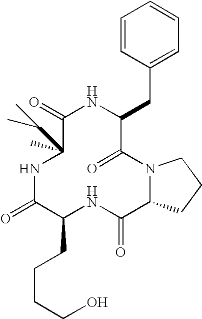

Compound (132)

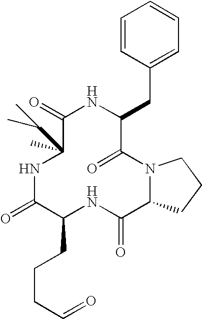

Compound (133)

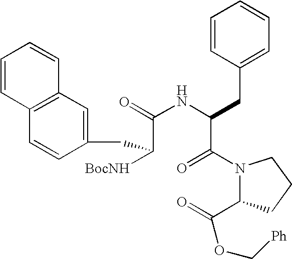

TABLE 2-17
Compound (134)

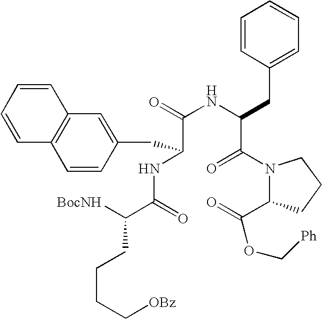

Compound (135)

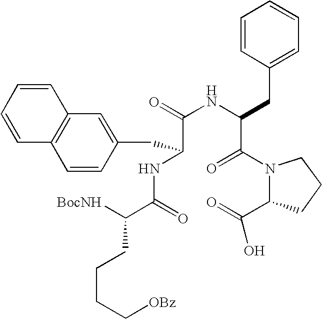

Compound (136)

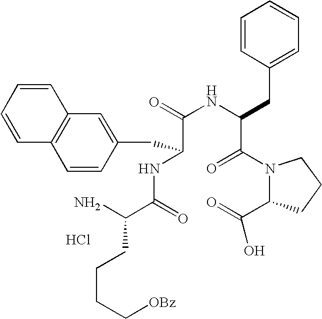

Compound (137)

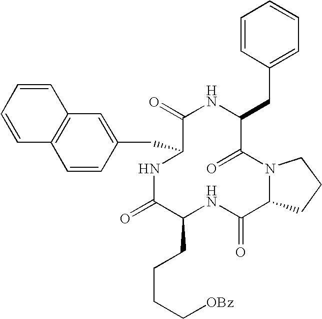

Compound (138)

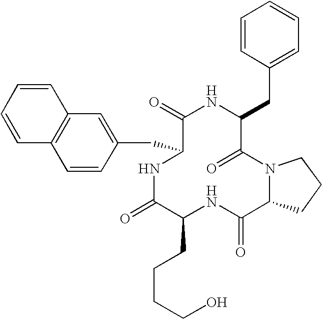

Compound (139)

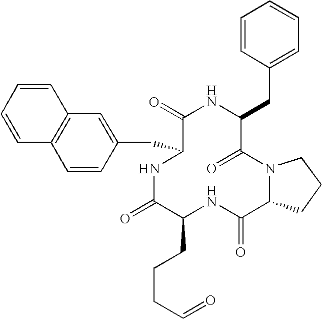

Compound (140)

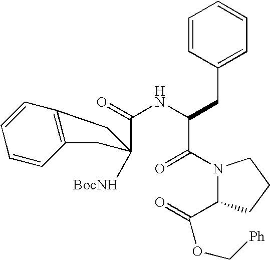

Compound (141)

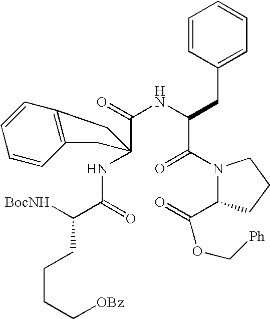

TABLE 2-18
Compound (142)

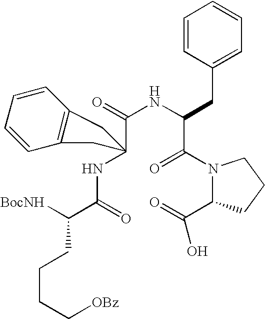

Compound (143)

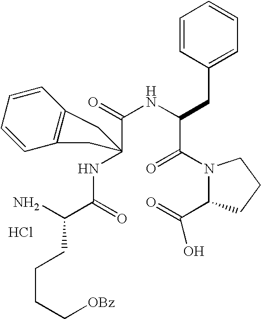

Compound (144)

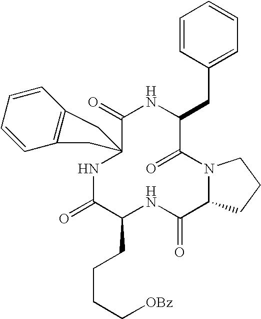

Compound (145)

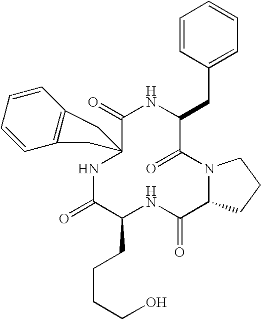

Compound (146)

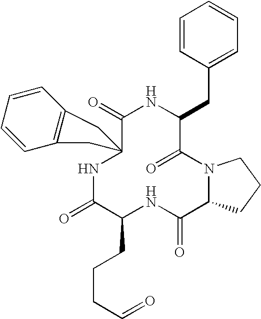

Compound (147)

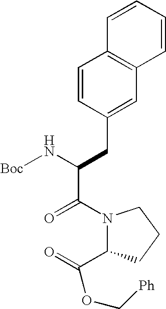

Compound (148)

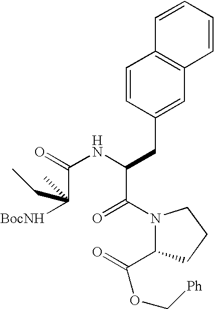

Compound (149)

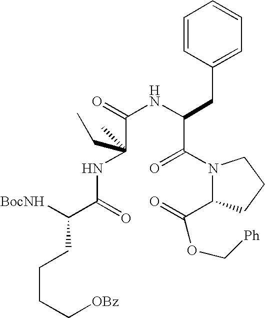

TABLE 2-19
Compound (150)

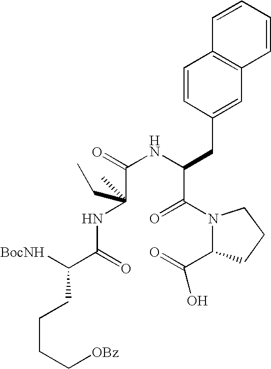

Compound (151)

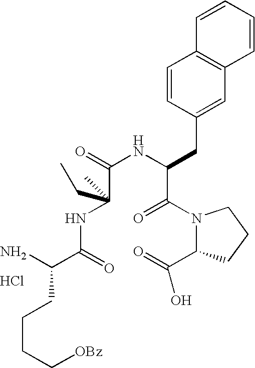

Compound (152)

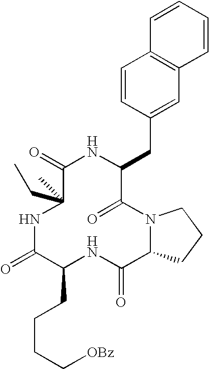

Compound (153)

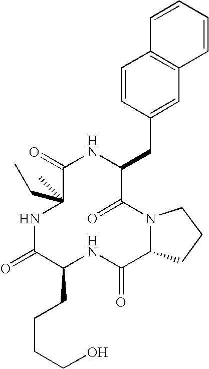

Compound (154)

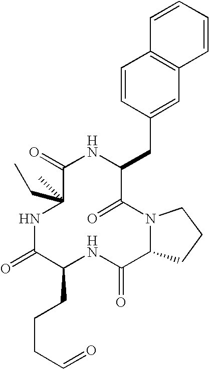

Compound (155)

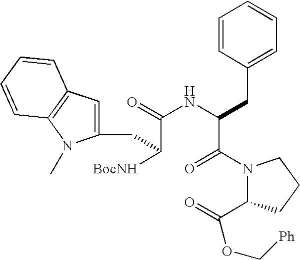

Compound (156)

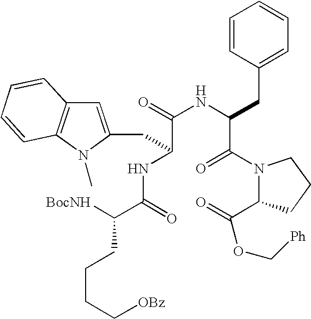

Compound (157)

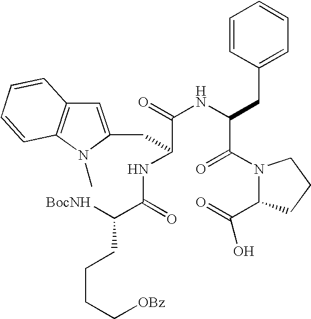

TABLE 2-20
Compound (158)

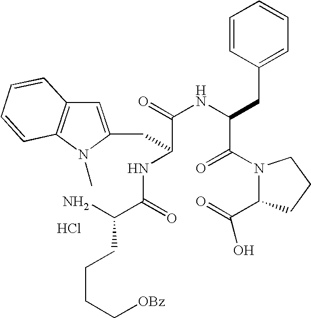

Compound (159)

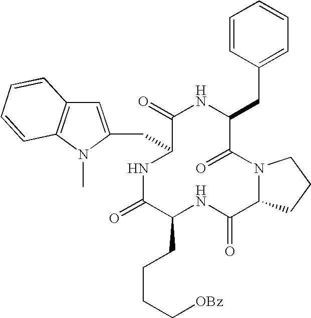

Compound (160)

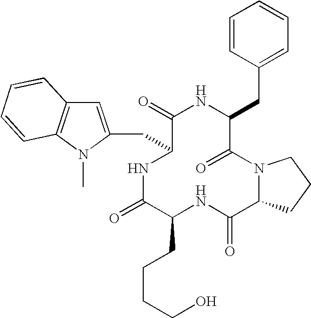

Compound (161)

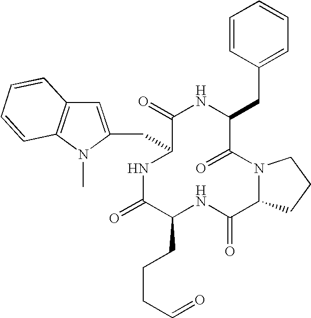

Compound (162)

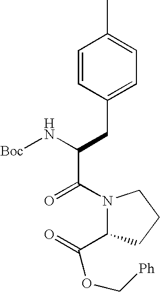

Compound (163)

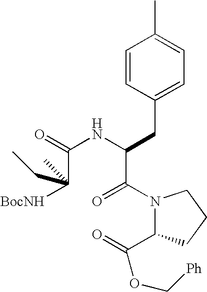

Compound (164)

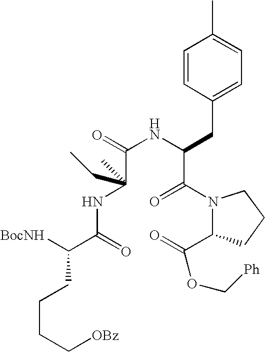

Compound (165)

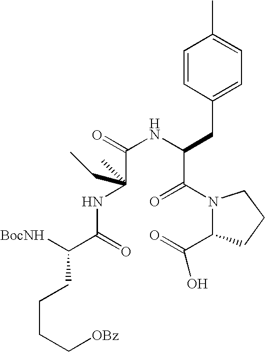

TABLE 2-21

| | |
|---|---|
| Compound (166) | Compound (167) |
| 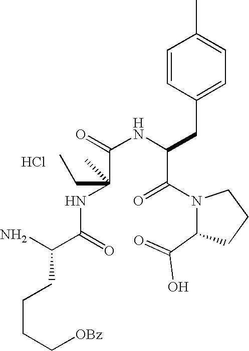 | 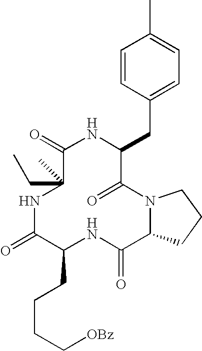 |
| Compound (168) | Compound (169) |
| 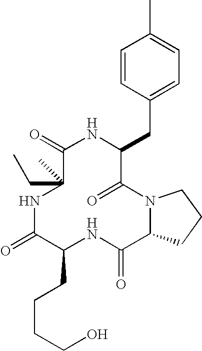 | 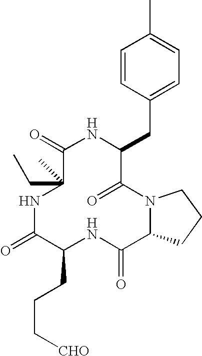 |
| Compound (170) | Compound (171) |
| 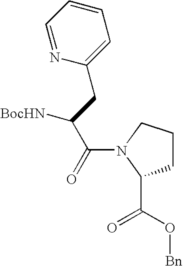 | 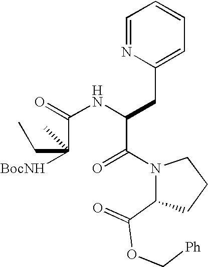 |
| Compound (172) | Compound (173) |
| 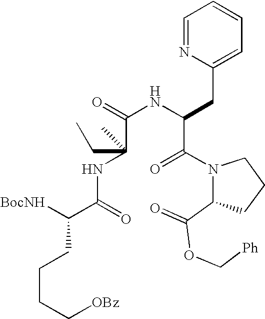 | 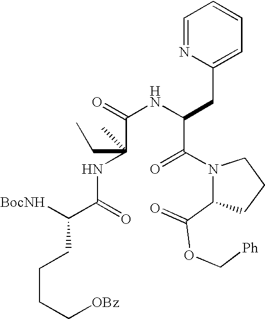 |

TABLE 2-22

| | |
|---|---|
| Compound (174) | Compound (175) |
| 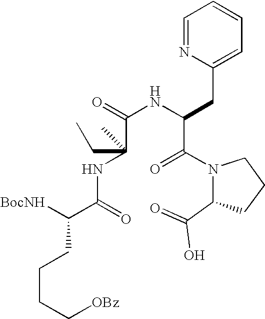 | 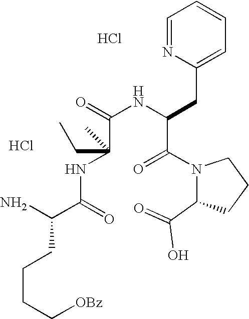 |
| Compound (176) | Compound (177) |
| 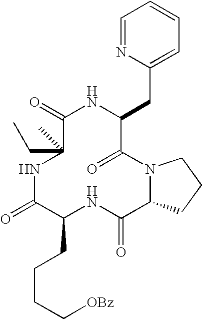 | 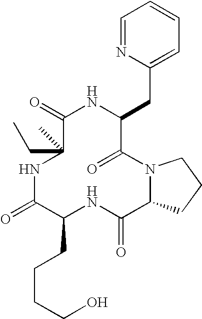 |
| Compound (178) | Compound (179) |
| 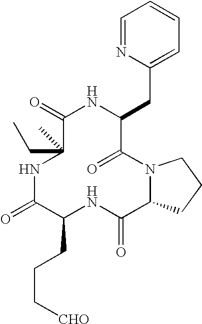 | 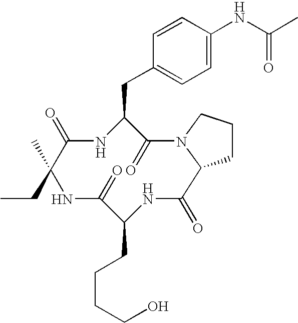 |
| Compound (180) | Compound (181) |
| 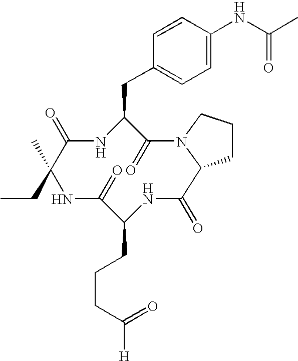 | 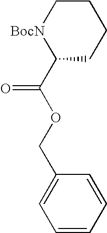 |

TABLE 2-23

| | |
|---|---|
| Compound (182) | Compound (183) |
| 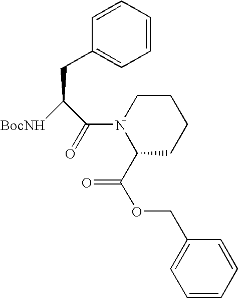 | 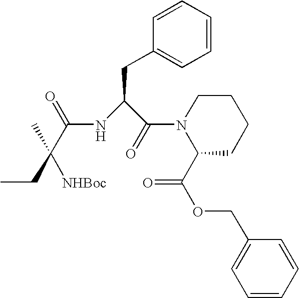 |
| Compound (184) | Compound (185) |
| 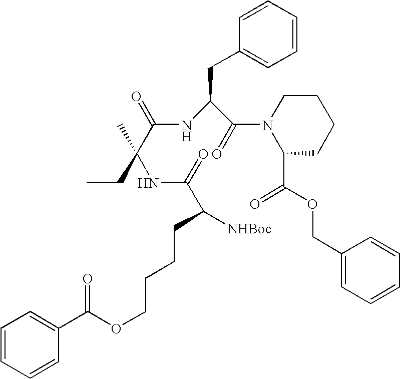 | 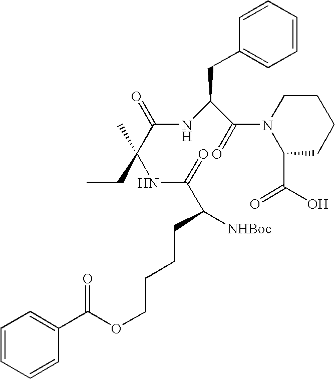 |
| Compound (186) | Compound (187) |
| 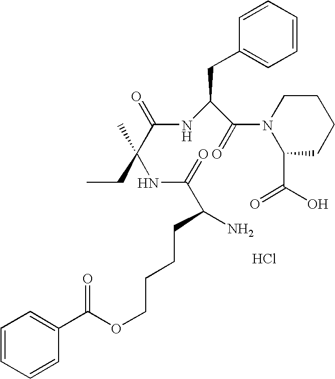 | 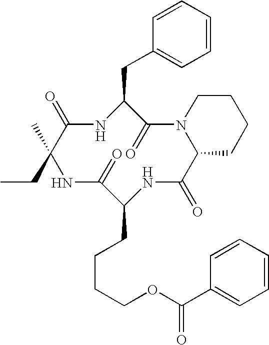 |
| Compound (188) | Compound (189) |
| 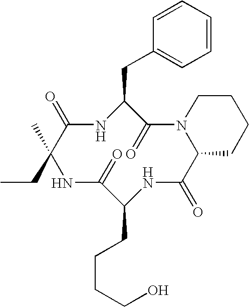 | 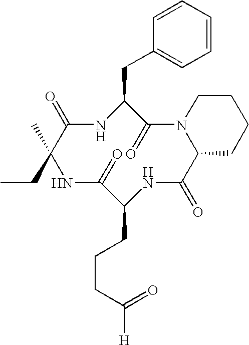 |

TABLE 2-24

| | |
|---|---|
| Compound (190) | Compound (191) |
| 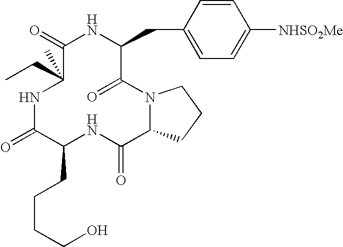 | 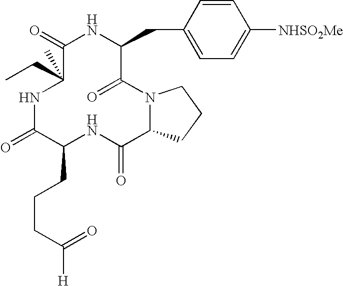 |
| Compound (192) | Compound (193) |
| 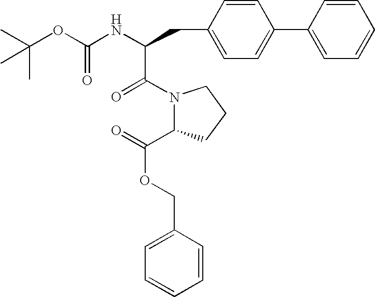 | 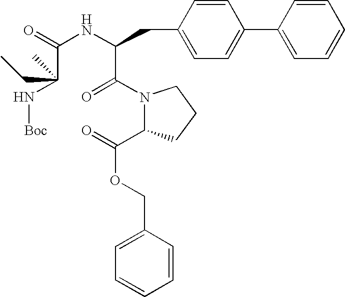 |
| Compound (194) | Compound (195) |
| 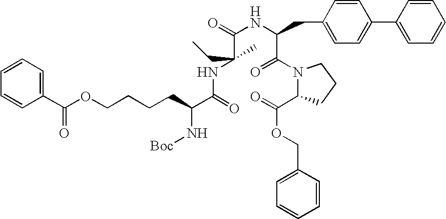 | 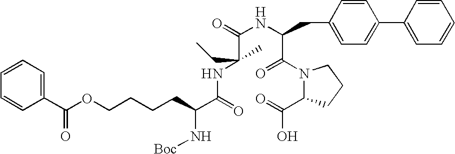 |
| Compound (196) | Compound (197) |
| 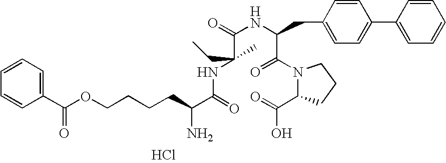 | 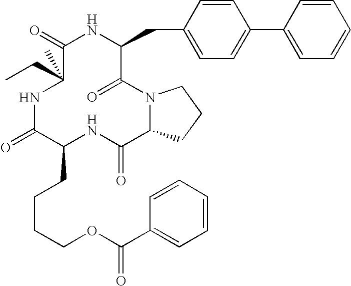 |

TABLE 2-25

| | |
|---|---|
| Compound (198) | Compound (199) |
| 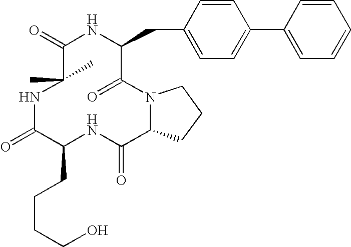 | 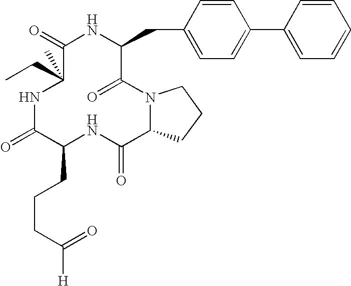 |
| Compound (200) | Compound (201) |
| 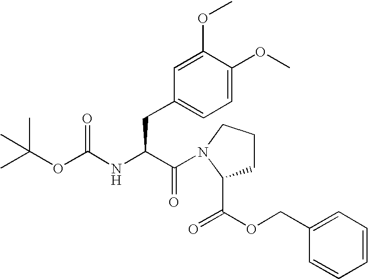 | 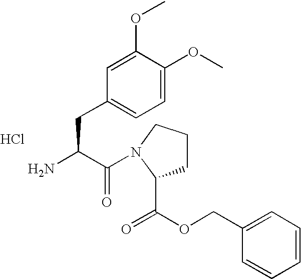 |
| Compound (202) | Compound (203) |
| 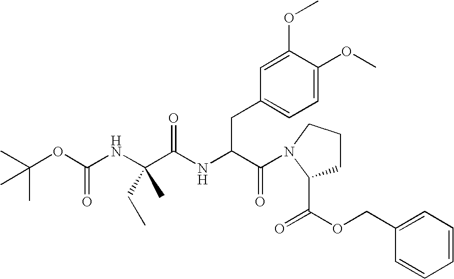 | 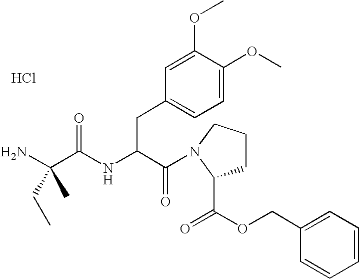 |
| Compound (204) | Compound (205) |
| 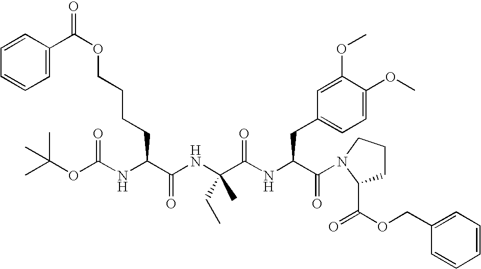 | 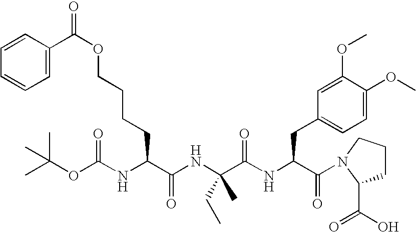 |

TABLE 2-26

| | |
|---|---|
| Compound (206) | Compound (207) |
| 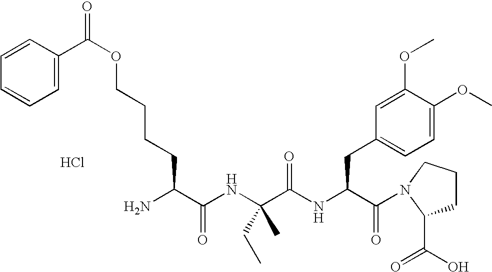 | 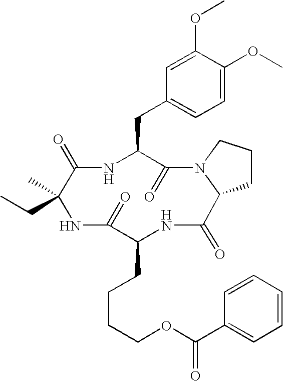 |
| Compound (208) | Compound (209) |
| 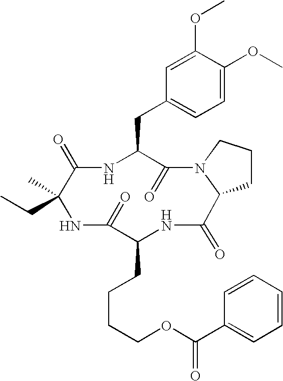 | 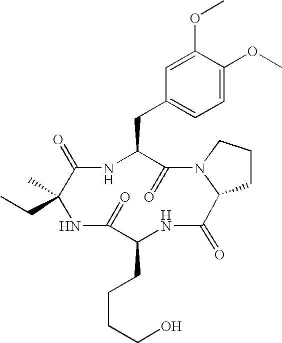 |
| Compound (210) | Compound (211) |
| 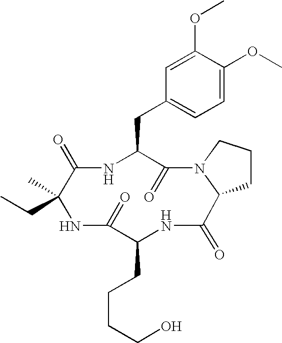 | 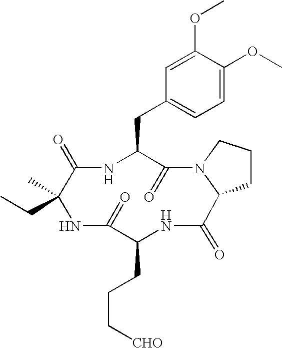 |
| Compound (212) | Compound (213) |
| 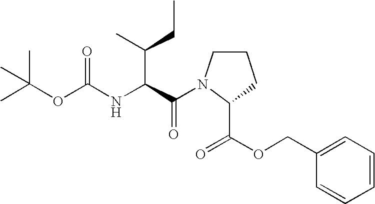 | 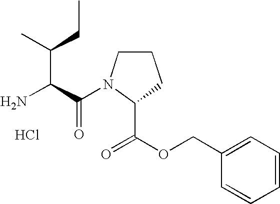 |

TABLE 2-27

| | |
|---|---|
| Compound (214) | Compound (215) |
| 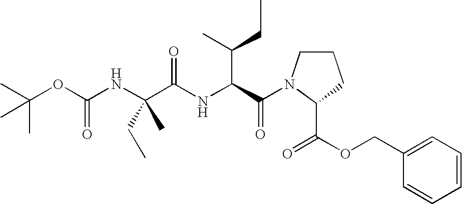 | 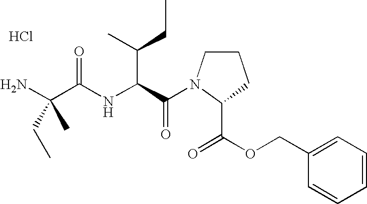 |
| Compound (216) | Compound (217) |
| 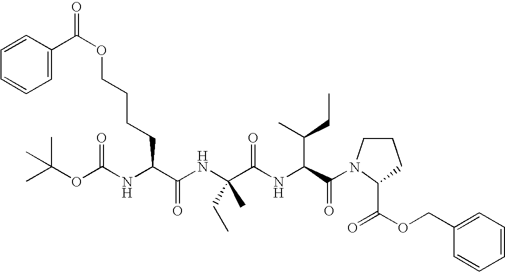 | 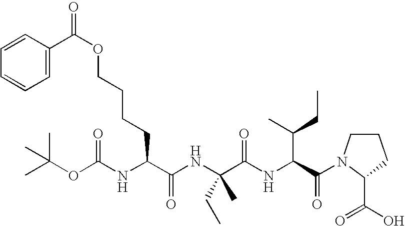 |
| Compound (218) | Compound (219) |
| 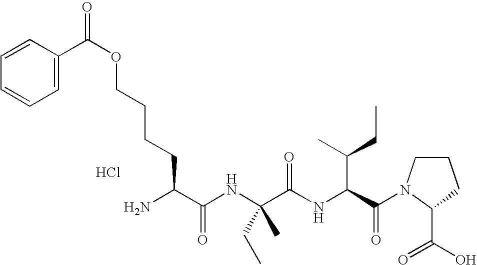 | get,0054 |
| Compound (220) | Compound (221) |
| 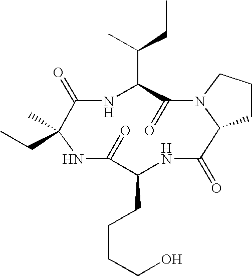 | 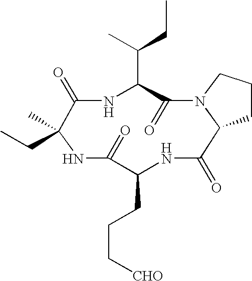 |

TABLE 2-28

| | |
|---|---|
| Compound (222) | Compound (223) |
| 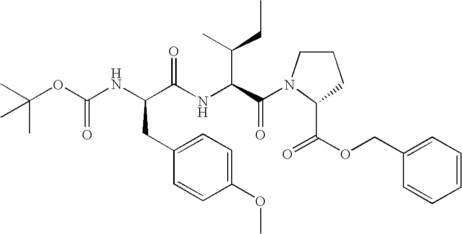 | 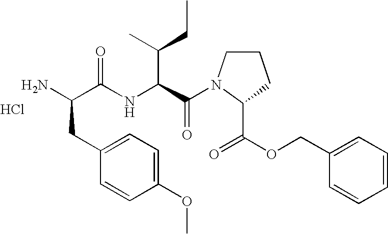 |
| Compound (224) | Compound (225) |
| 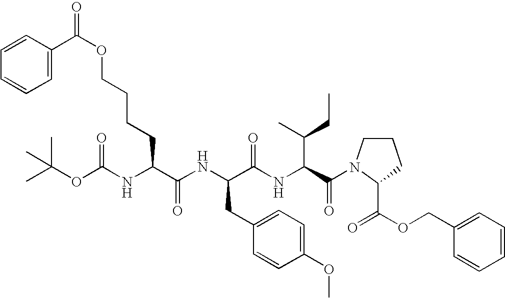 | 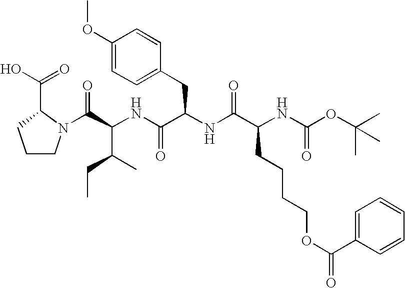 |
| Compound (226) | Compound (227) |
| 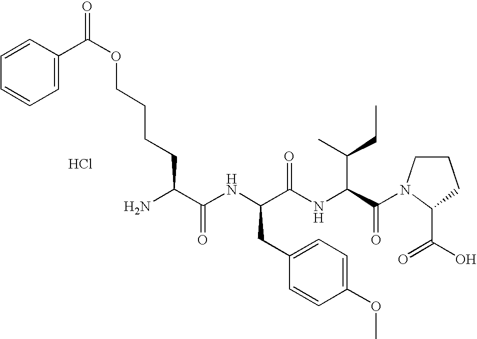 | 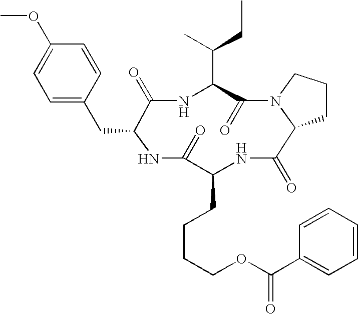 |
| Compound (228) | Compound (229) |
| 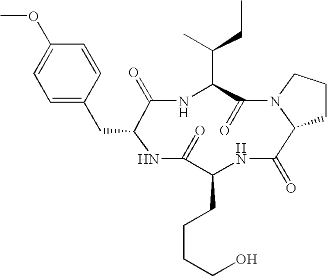 | 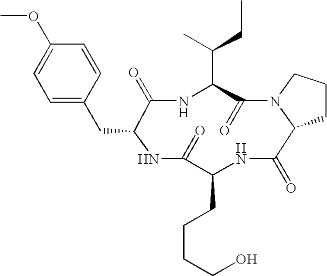 |

TABLE 2-29

| | |
|---|---|
| Compound (230) | Compound (231) |
| 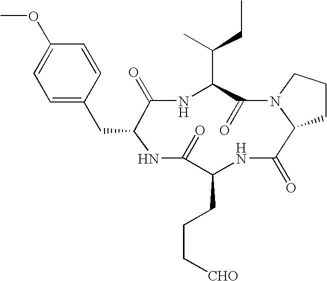 | 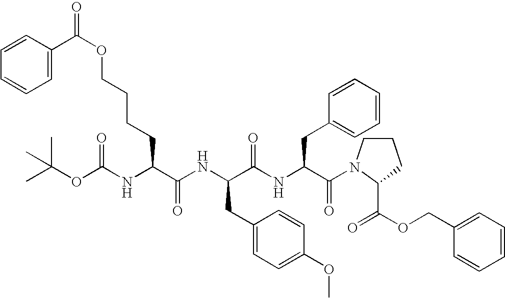 |
| Compound (232) | Compound (233) |
| 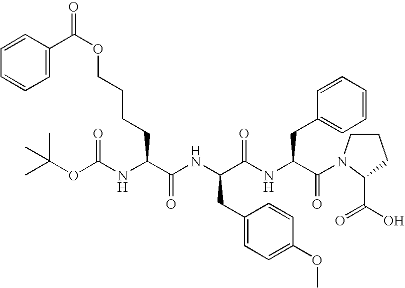 | 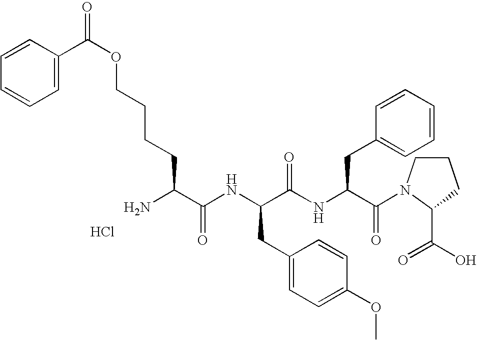 |
| Compound (234) | Compound (235) |
| 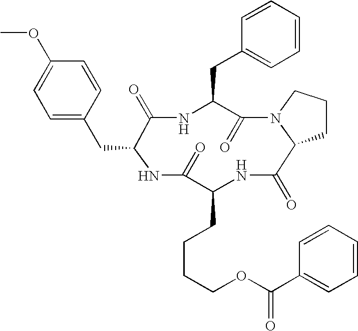 | 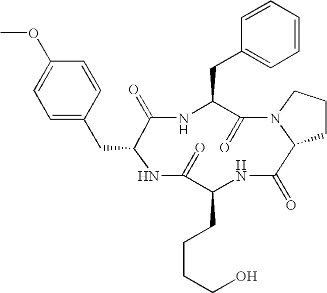 |
| Compound (236) | Compound (237) |
| 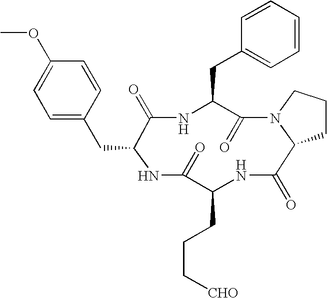 | 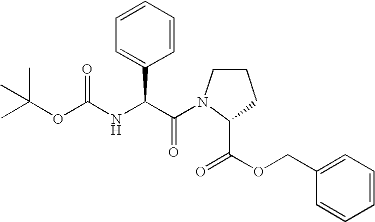 |

TABLE 2-30

| | |
|---|---|
| Compound (238) | Compound (239) |
| 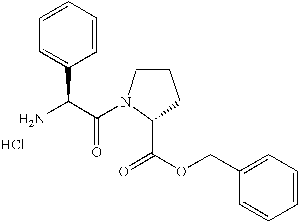 | 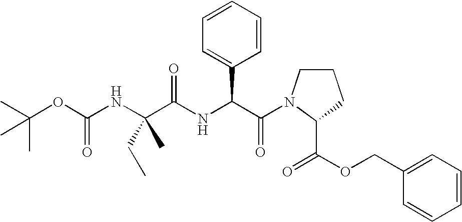 |
| Compound (240) | Compound (241) |
| 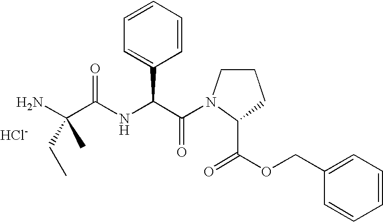 | 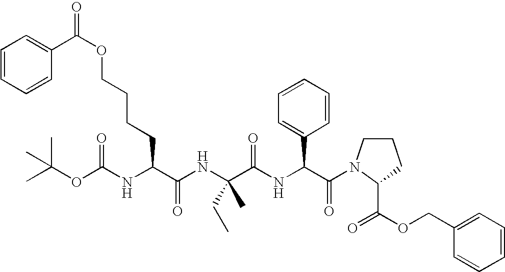 |
| Compound (242) | Compound (243) |
| 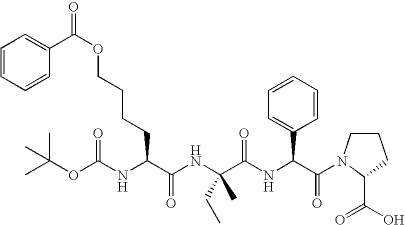 | 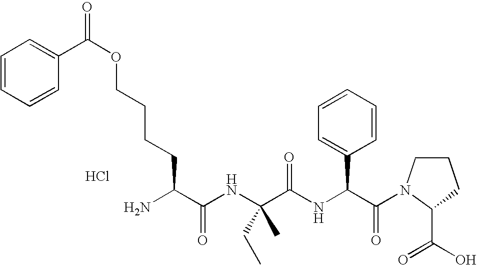 |
| Compound (244) | Compound (245) |
| 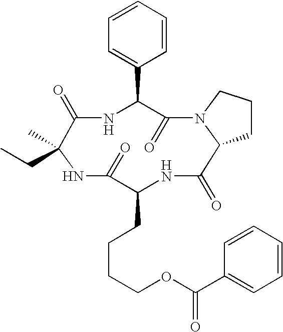 | 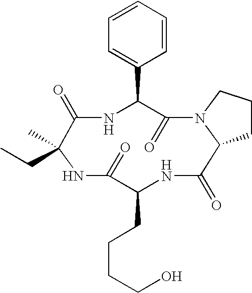 |

TABLE 2-31
Compound (246)

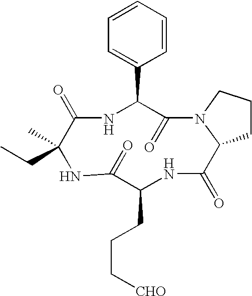

Compound (247)

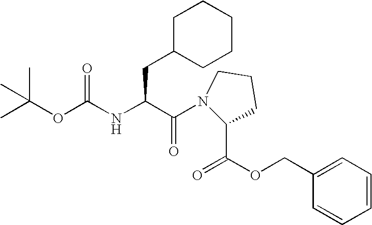

Compound (248)

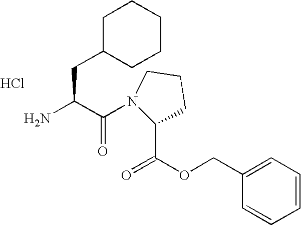

Compound (249)

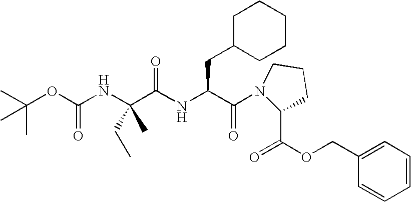

Compound (250)

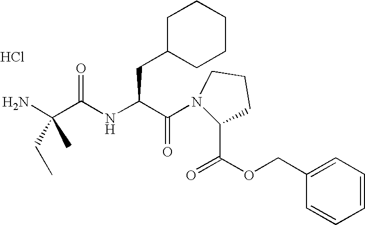

Compound (251)

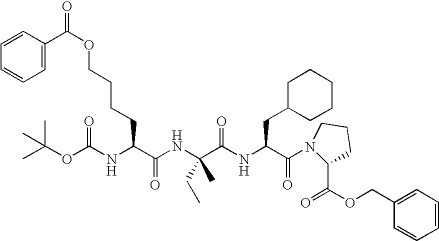

Compound (252)

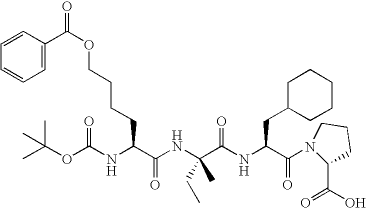

Compound (253)

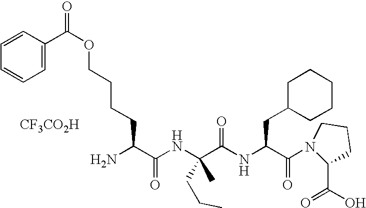

TABLE 2-32
Compound (254)

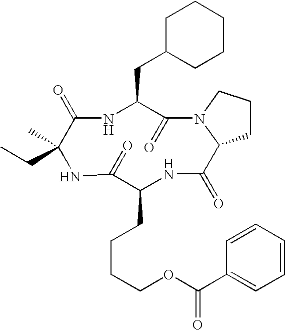

Compound (255)

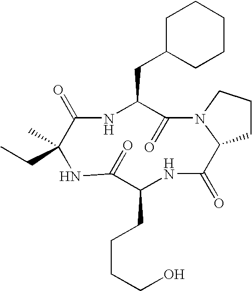

Compound (256)

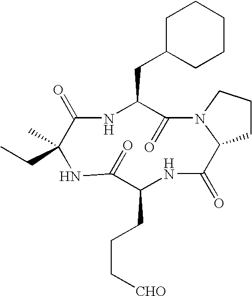

Compound (257)

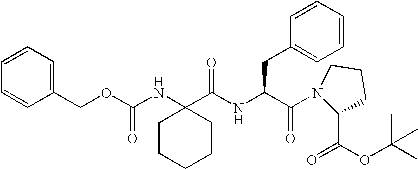

Compound (258)

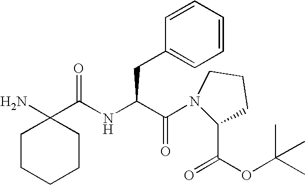

Compound (259)

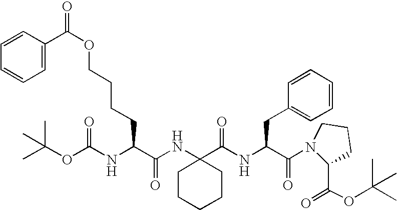

Compound (260)

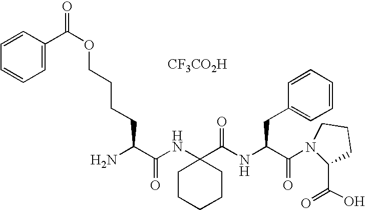

Compound (261)

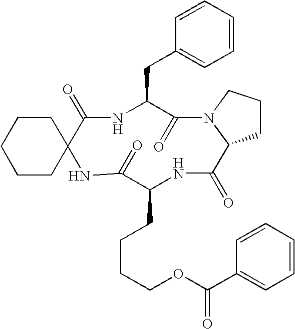

TABLE 2-33
Compound (262)

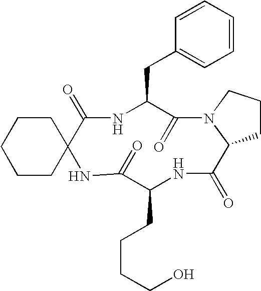

Compound (263)

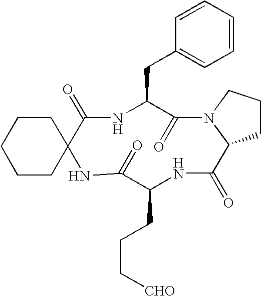

Compound (264)

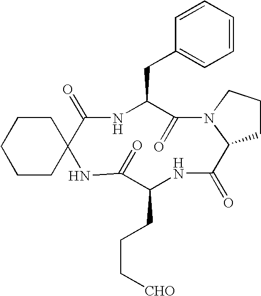

Compound (265)

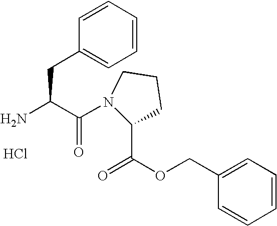

Compound (266)

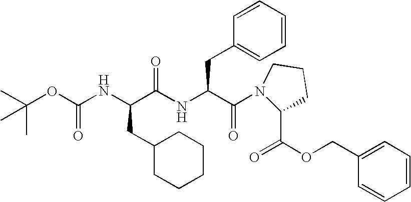

Compound (267)

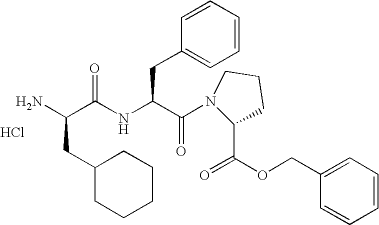

Compound (268)

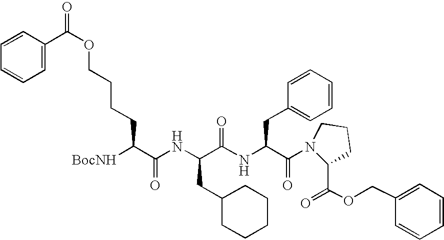

Compound (269)

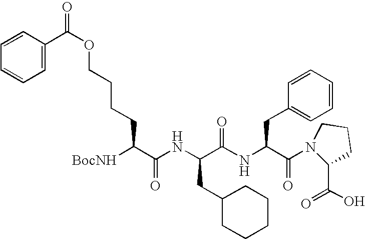

TABLE 2-34
Compound (270)

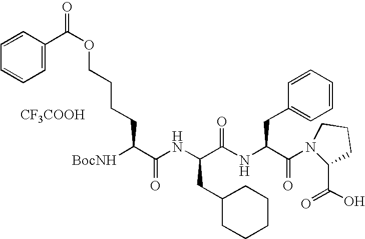

Compound (271)

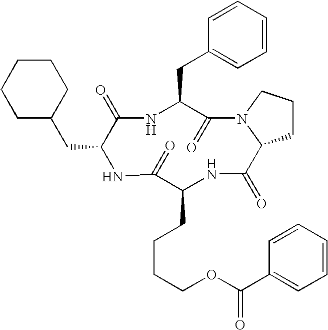

Compound (272)

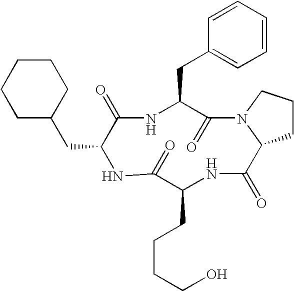

Compound (273)

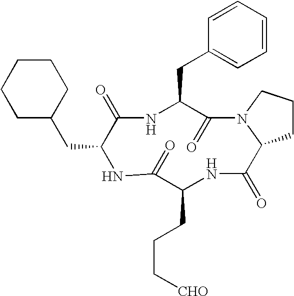

Compound (274)

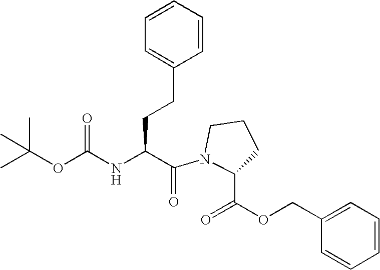

Compound (275)

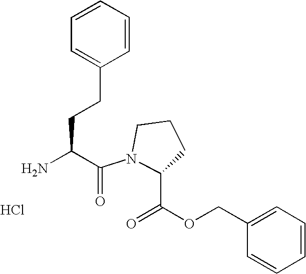

Compound (276)

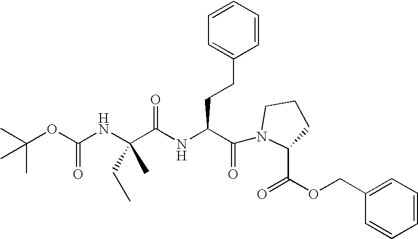

Compound (277)

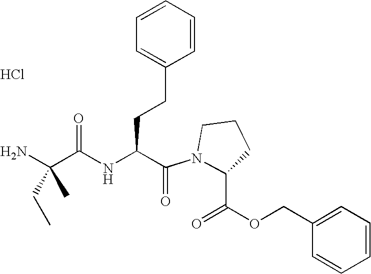

TABLE 2-35
Compound (278)

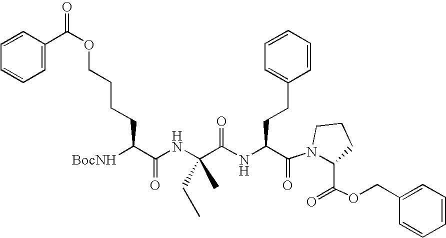

Compound (279)

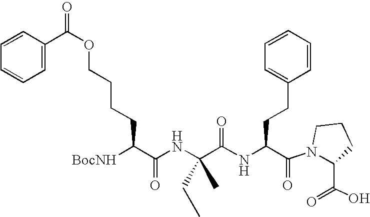

Compound (280)

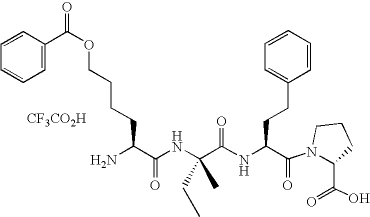

Compound (281)

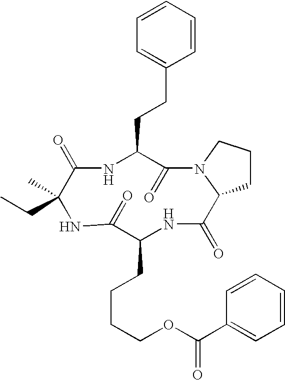

Compound (282)

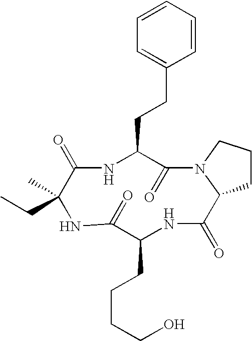

Compound (283)

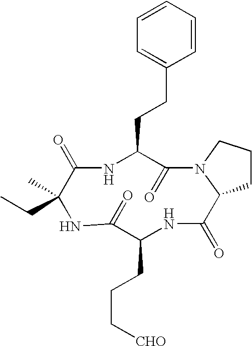

Compound (284)

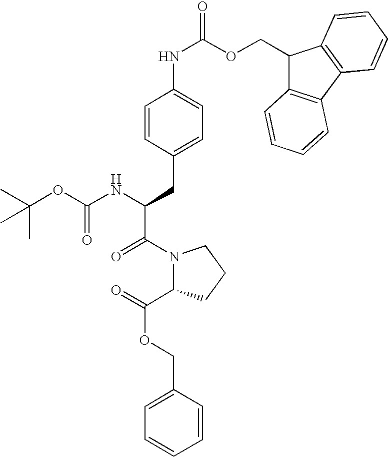

Compound (285)

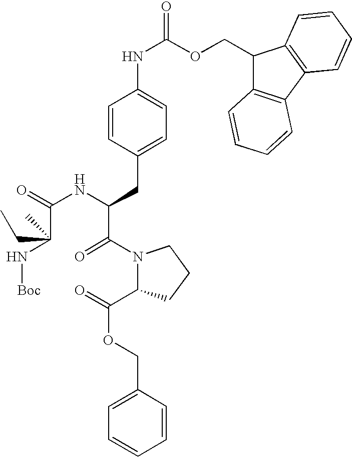

TABLE 2-36
Compound (286)

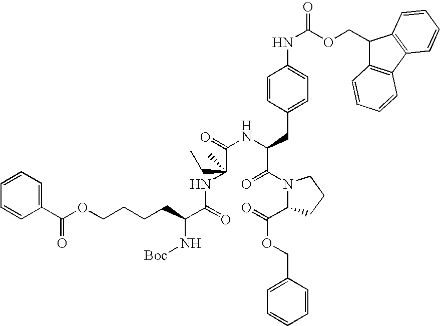

Compound (287)

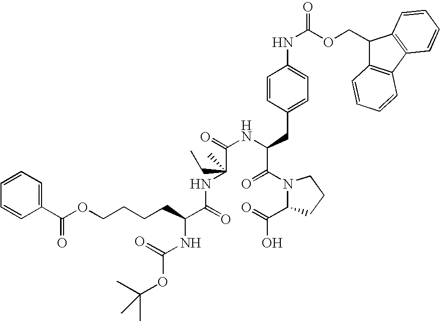

Compound (288)

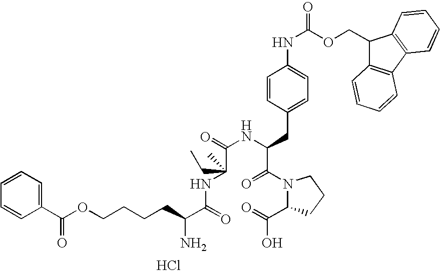

Compound (289)

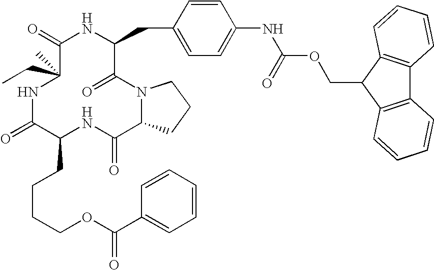

Compound (290)

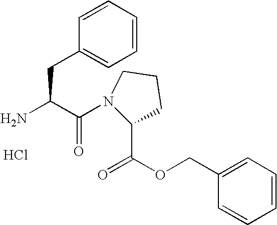

Compound (291)

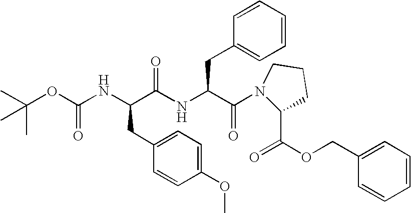

Compound (292)

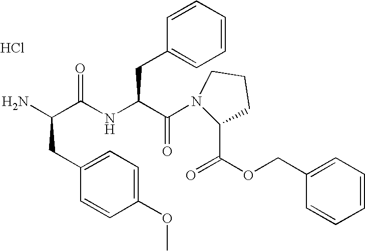

Compound (293)

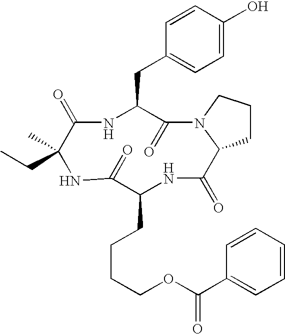

TABLE 2-37
Compound (294)

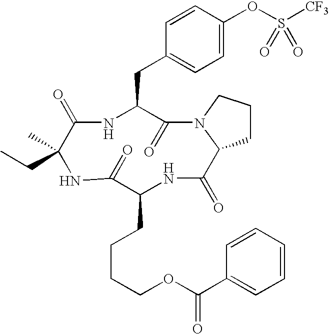

Compound (295)

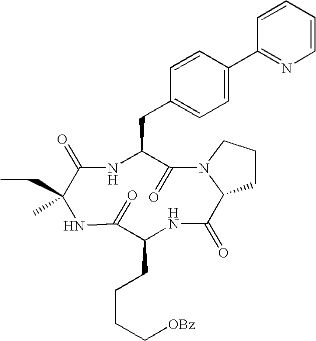

Compound (296)

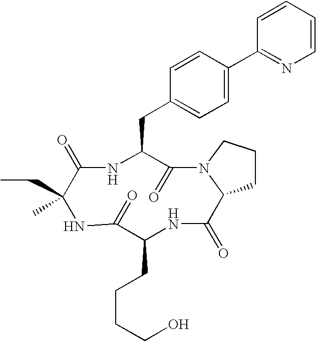

Compound (297)

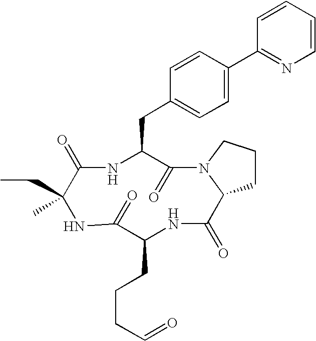

Compound (298)

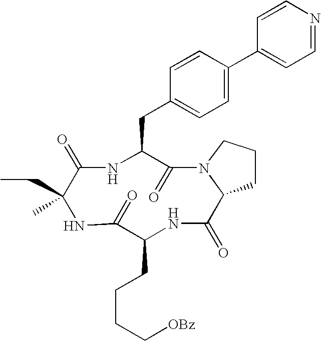

Compound (299)

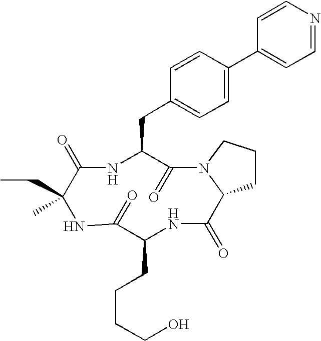

Compound (300)

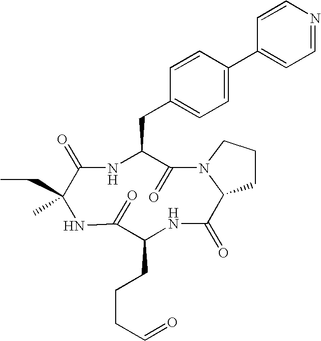

Compound (301)

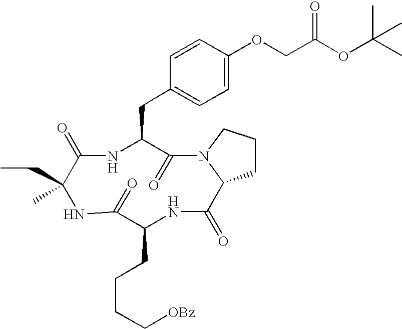

TABLE 2-38
Compound (302)

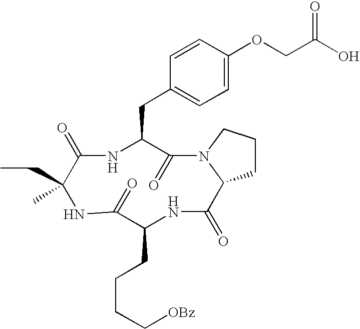

Compound (303)

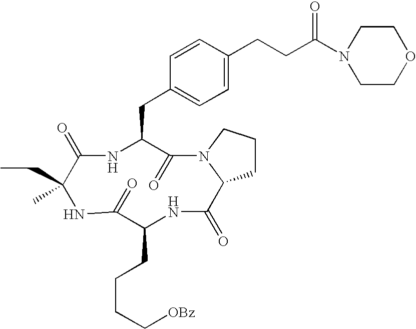

Compound (304)

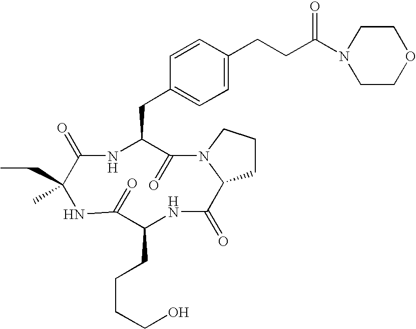

Compound (305)

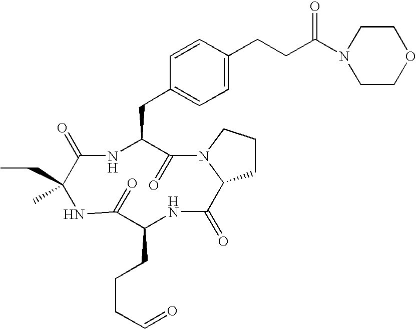

Compound (306)

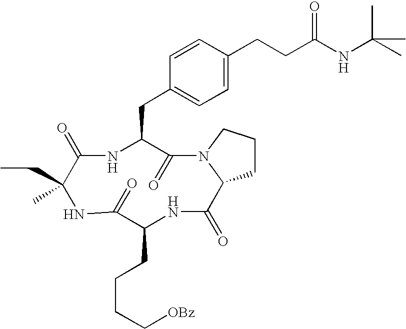

Compound (307)

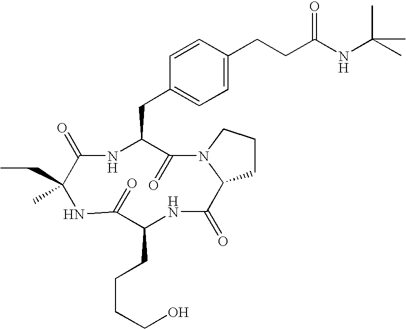

Compound (308)

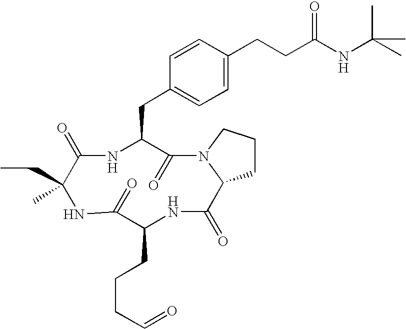

Compound (309)

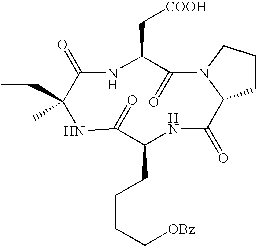

TABLE 2-39
Compound (310)

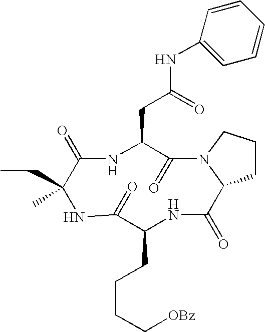

Compound (311)

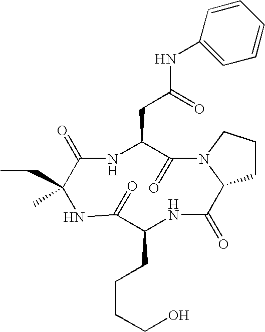

Compound (312)

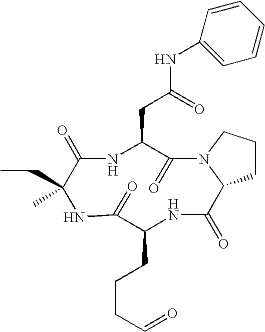

Compound (313)

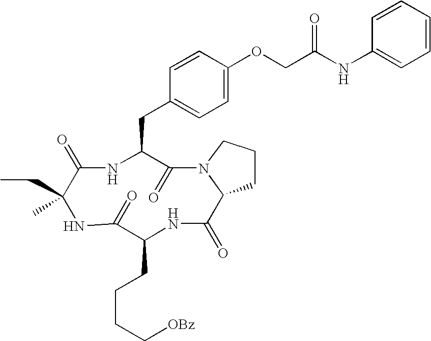

Compound (314)

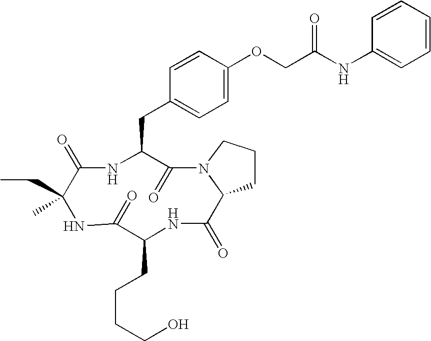

Compound (315)

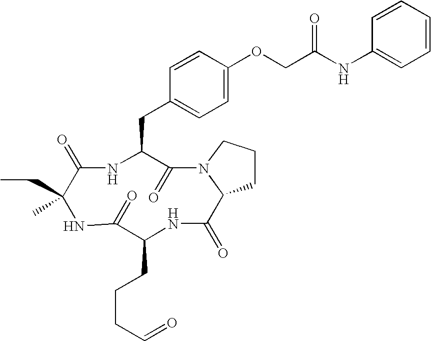

Compound (316)

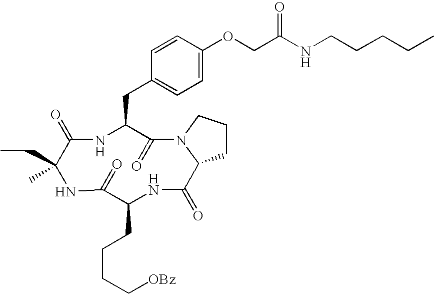

Compound (317)

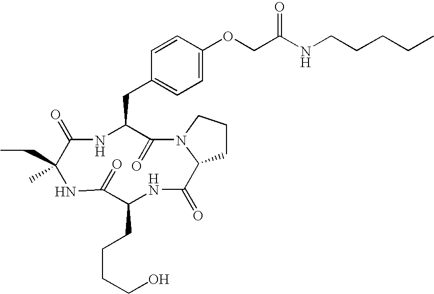

TABLE 2-40
Compound (318)

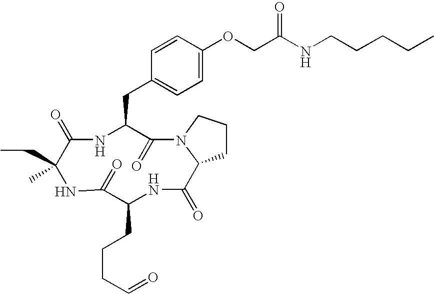

Compound (319)

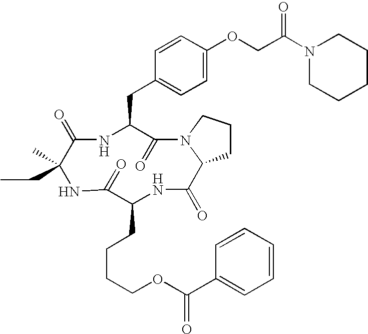

Compound (320)

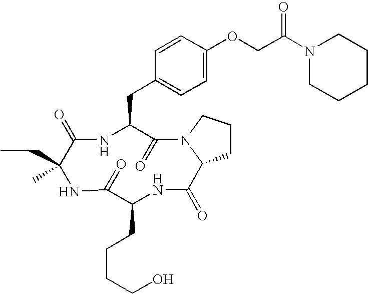

Compound (321)

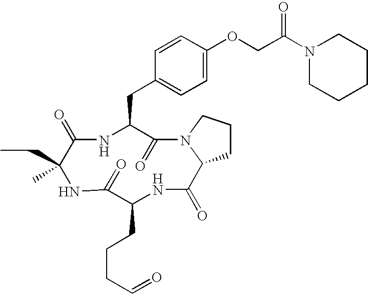

Compound (322)

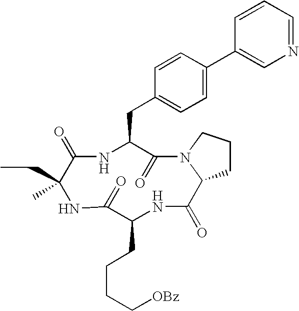

Compound (323)

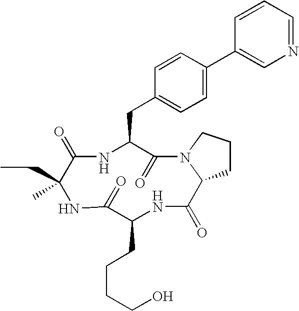

Compound (324)

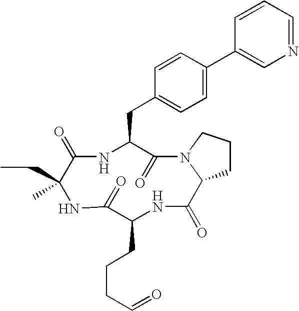

Compound (325)

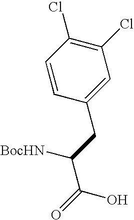

TABLE 2-41

| | |
|---|---|
| Compound (326) | Compound (327) |
| 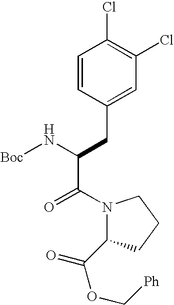 | 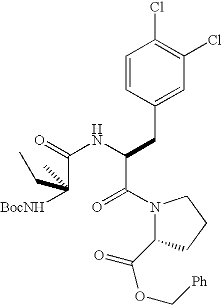 |
| Compound (328) | Compound (329) |
| 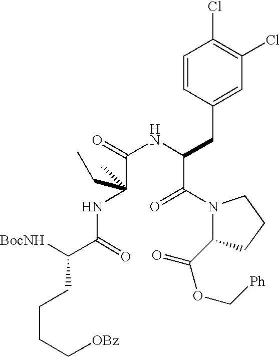 | 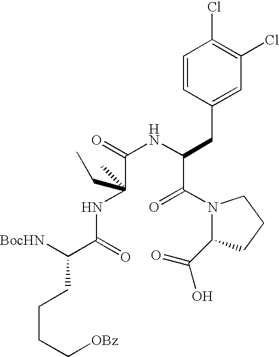 |
| Compound (330) | Compound (331) |
| 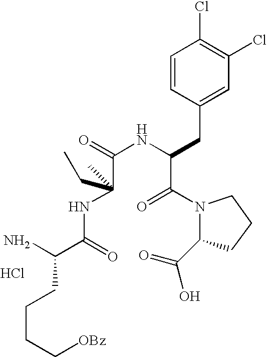 | 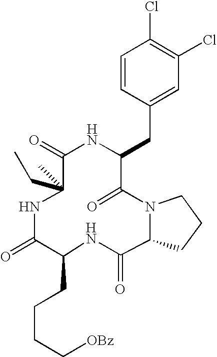 |
| Compound (332) | Compound (333) |
| 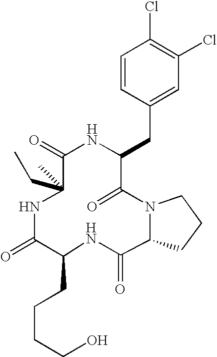 | 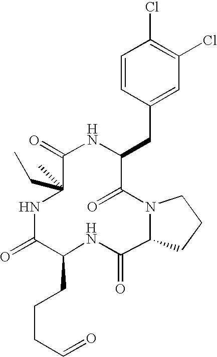 |

TABLE 2-42

| | |
|---|---|
| Compound (334) | Compound (335) |
| 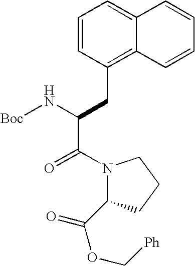 | 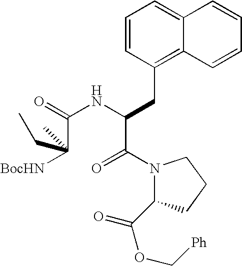 |
| Compound (336) | Compound (337) |
| 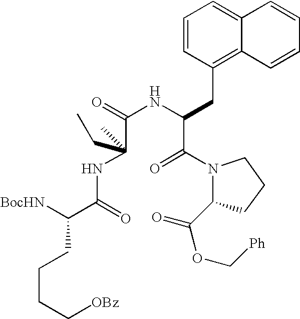 | 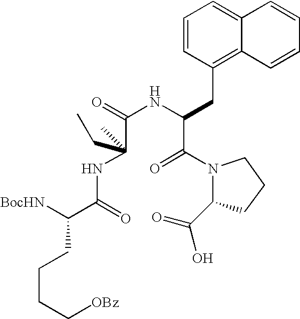 |
| Compound (338) | Compound (339) |
| 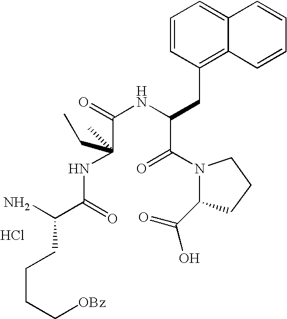 | 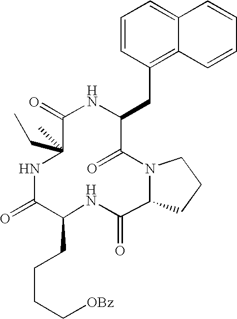 |
| Compound (340) | Compound (341) |
| 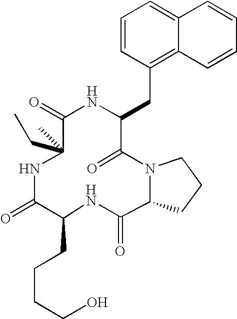 | 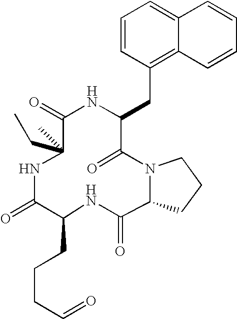 |

TABLE 2-43

| | |
|---|---|
| Compound (342) | Compound (343) |
| 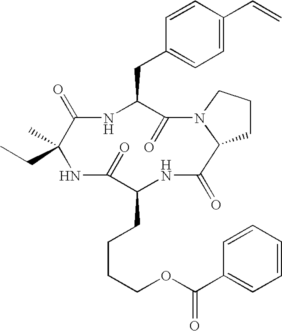 | 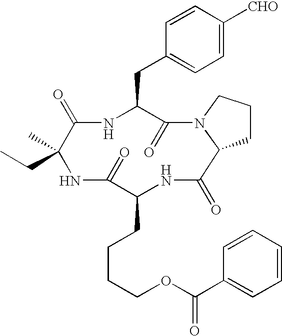 |
| Compound (344) | Compound (345) |
| 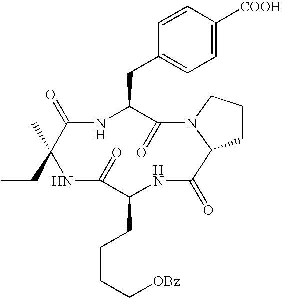 | 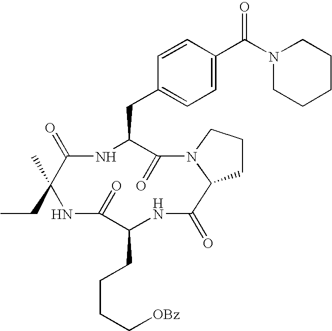 |
| Compound (346) | Compound (347) |
| 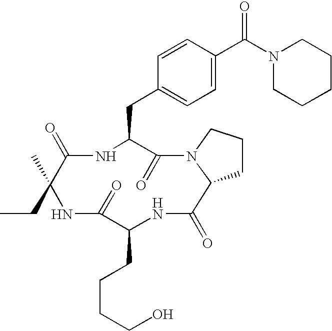 | 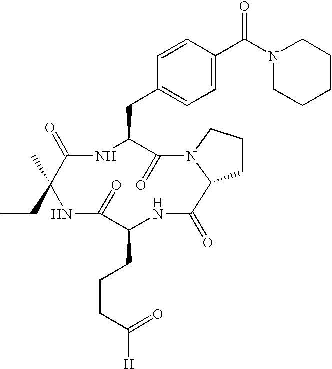 |
| Compound (348) | Compound (349) |
| 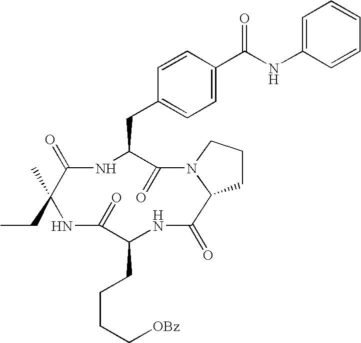 | 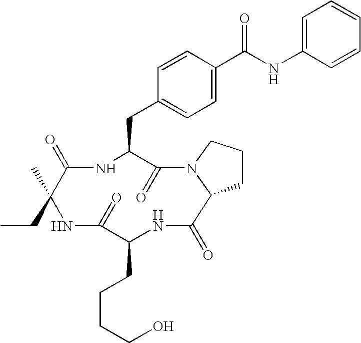 |

TABLE 2-44

| | |
|---|---|
| Compound (350) | Compound (351) |
| 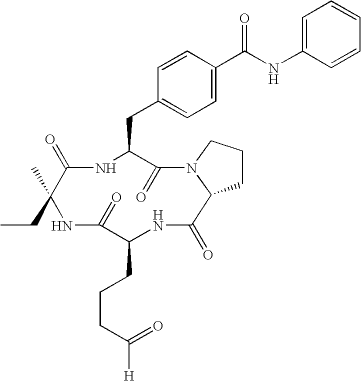 | 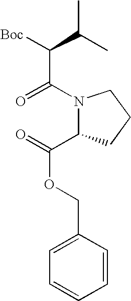 |
| Compound (352) | Compound (353) |
| 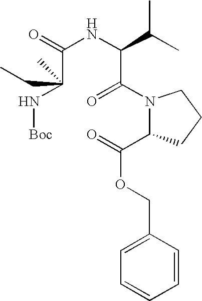 | 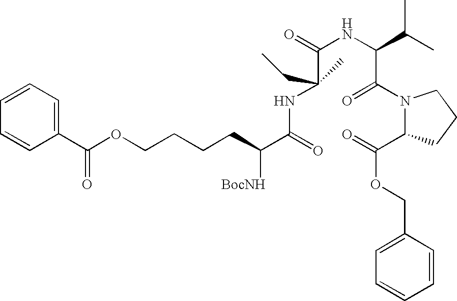 |
| Compound (354) | Compound (355) |
| 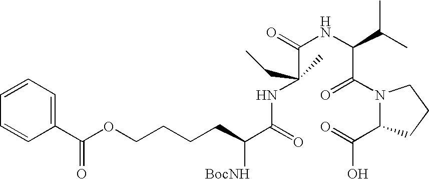 | 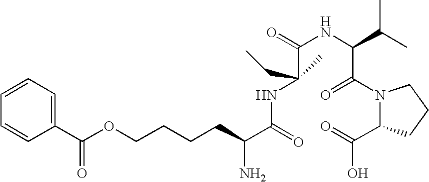 |
| Compound (356) | Compound (357) |
| 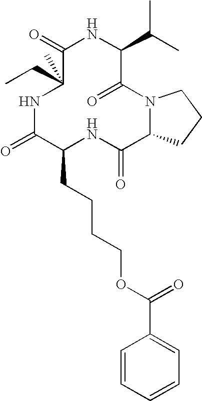 | 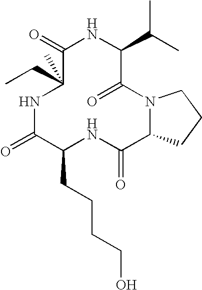 |

TABLE 2-45

| | |
|---|---|
| Compound (358) | Compound (359) |
| 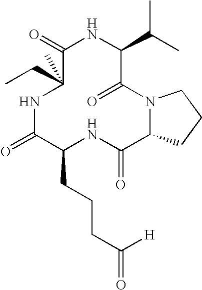 | 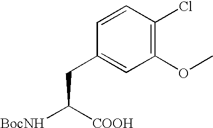 |
| Compound (360) | Compound (361) |
| 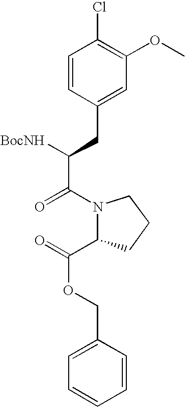 | 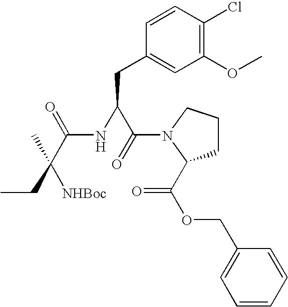 |
| Compound (362) | Compound (363) |
| 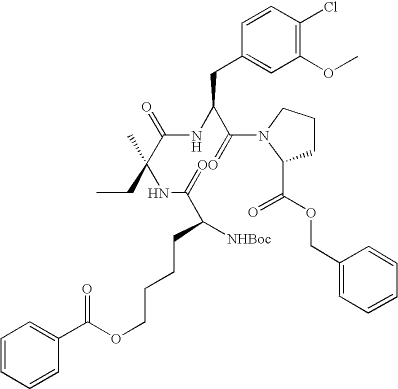 | 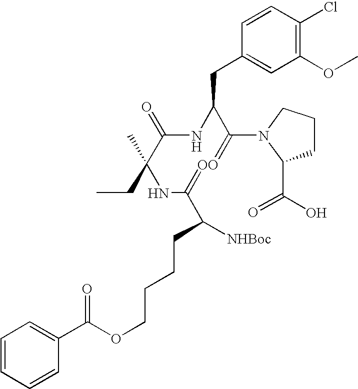 |
| Compound (364) | Compound (365) |
| 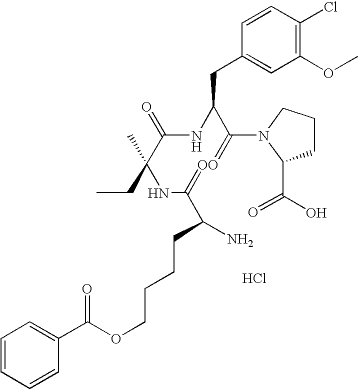 | 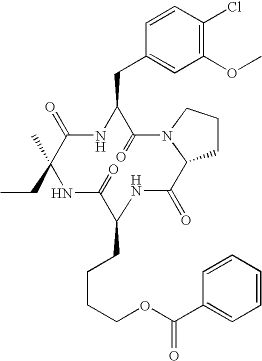 |

TABLE 2-46

| | |
|---|---|
| Compound (366) | Compound (367) |
| 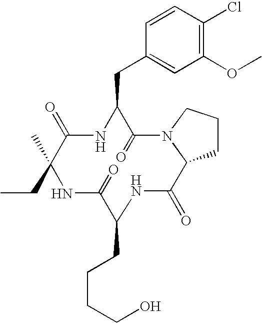 | 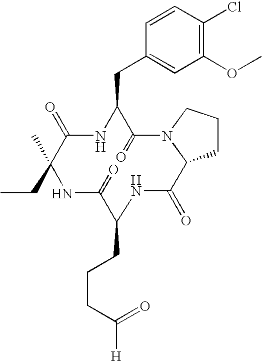 |
| Compound (368) | Compound (369) |
| 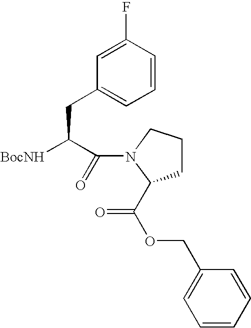 | 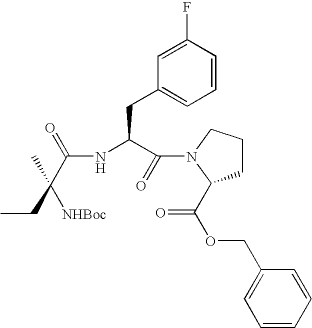 |
| Compound (370) | Compound (371) |
| 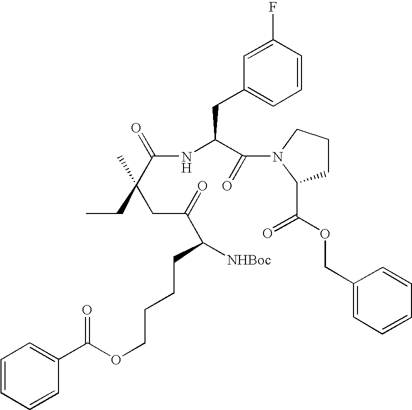 | 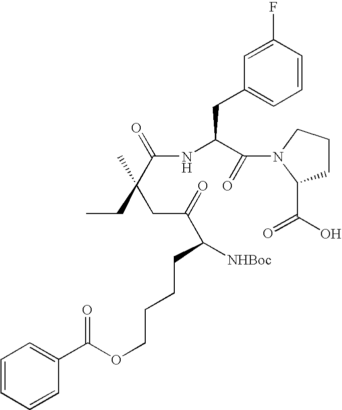 |
| Compound (372) | Compound (373) |
| 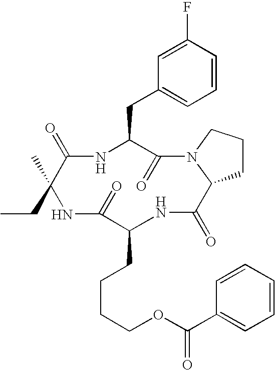 | 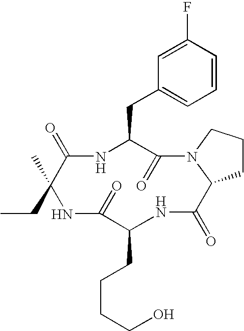 |

TABLE 2-47

| | |
|---|---|
| Compound (374) | Compound (375) |
| 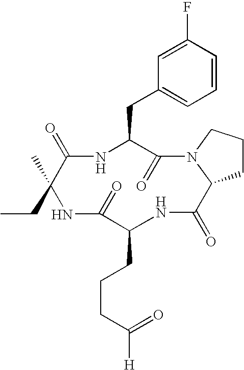 | 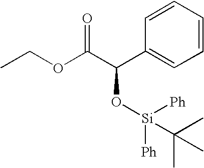 |
| Compound (376) | Compound (377) |
| 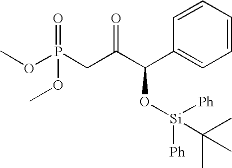 | 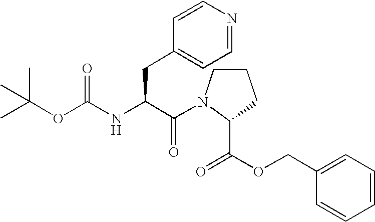 |
| Compound (378) | Compound (379) |
| 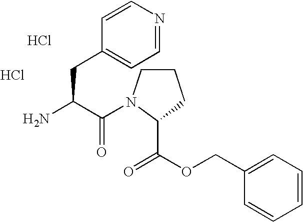 | 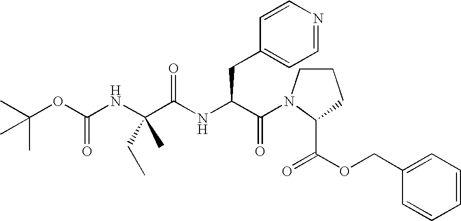 |
| Compound (380) | Compound (381) |
| 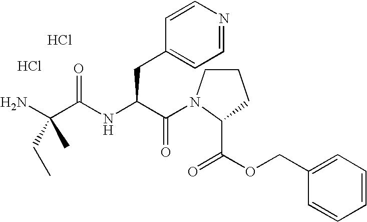 | 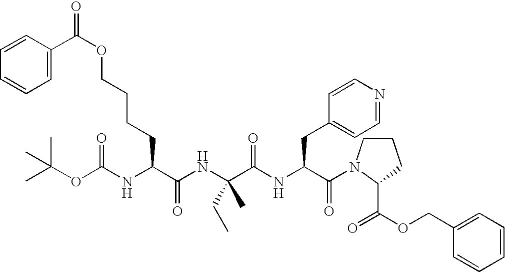 |

TABLE 2-48

| | |
|---|---|
| Compound (382) | Compound (383) |
| 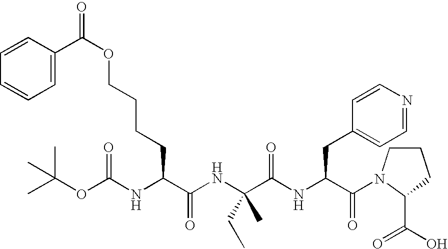 | 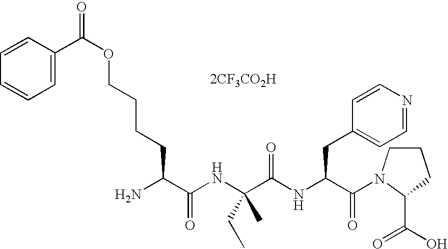 |
| Compound (384) | Compound (385) |
| 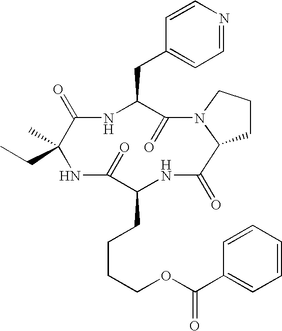 | 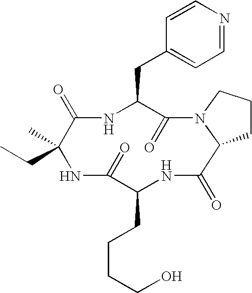 |
| Compound (386) | Compound (387) |
| 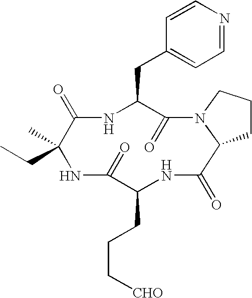 | 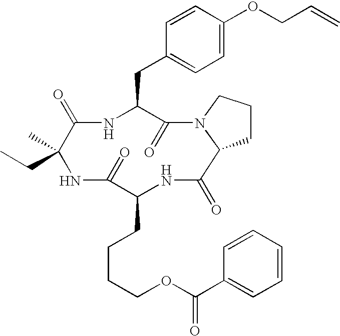 |
| Compound (388) | Compound (389) |
| 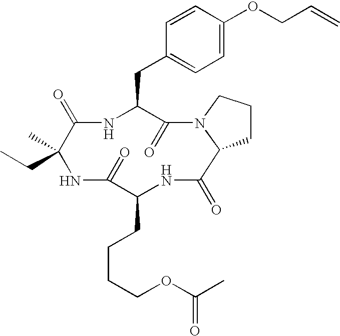 | 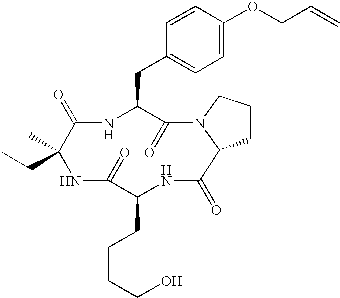 |

TABLE 2-49

| | |
|---|---|
| Compound (390) | Compound (391) |
| 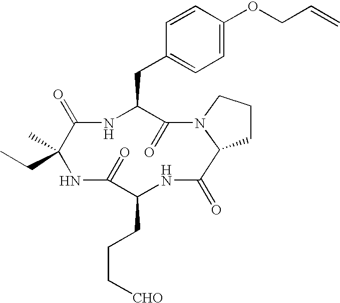 | 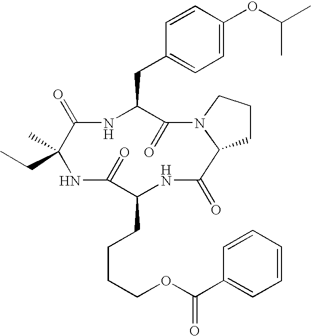 |
| Compound (392) | Compound (393) |
| 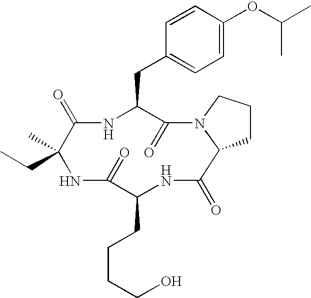 | 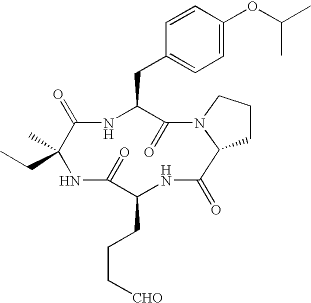 |
| Compound (394) | Compound (395) |
| 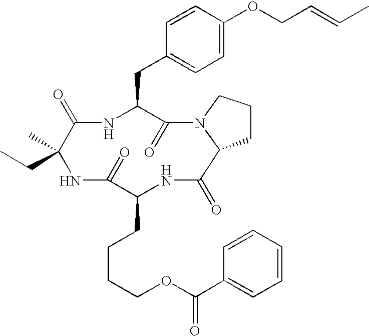 | 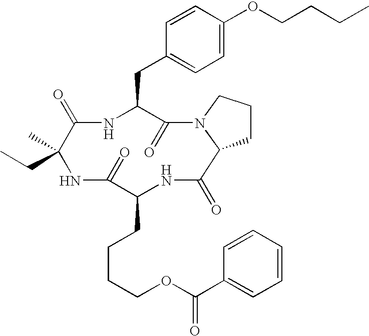 |
| Compound (396) | Compound (397) |
| 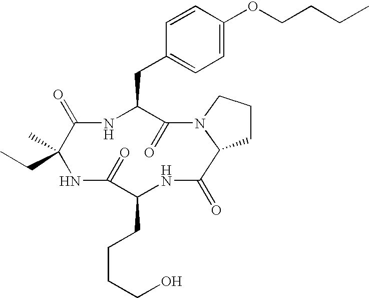 | 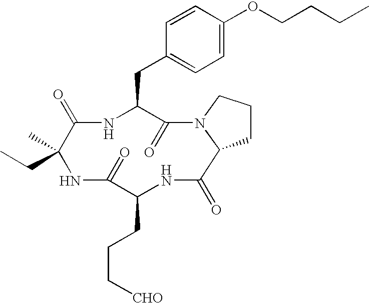 |

TABLE 2-50

| | |
|---|---|
| Compound (398) | Compound (399) |
| 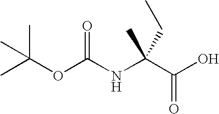 | 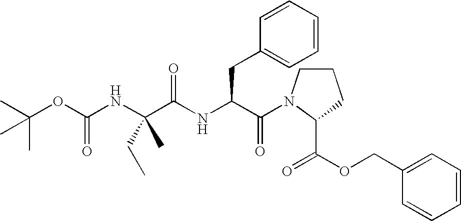 |
| Compound (400) | Compound (401) |
| 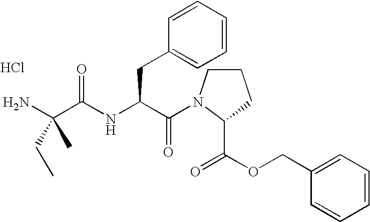 | 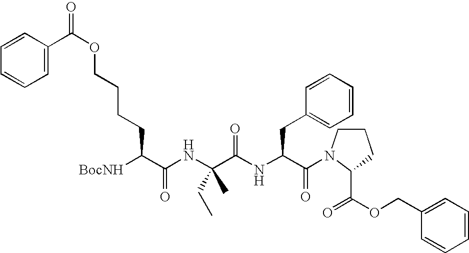 |
| Compound (402) | Compound (403) |
| 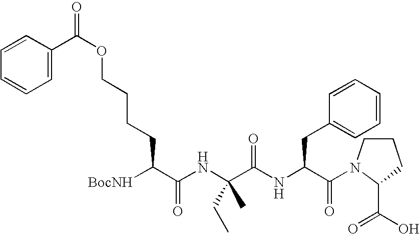 | 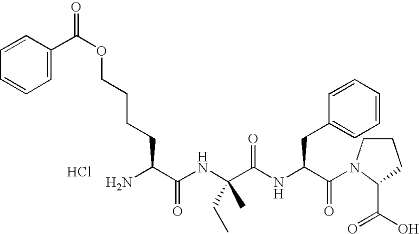 |
| Compound (404) | Compound (405) |
| 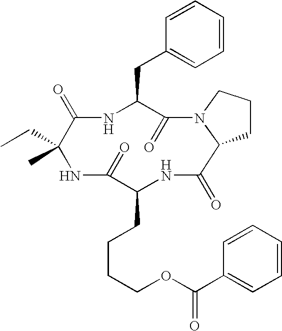 | 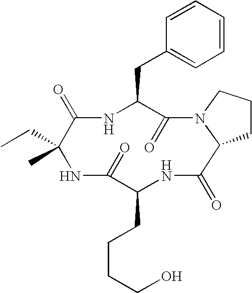 |

TABLE 2-51

| | |
|---|---|
| Compound (406) | Compound (407) |
| 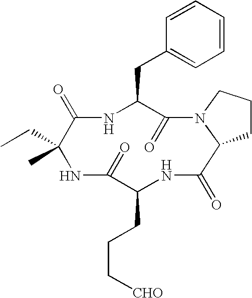 | 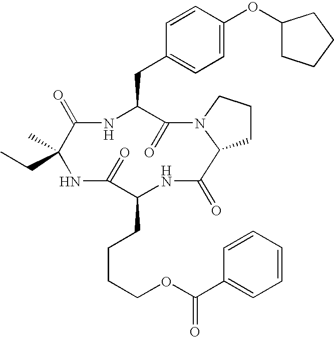 |
| Compound (408) | Compound (409) |
| 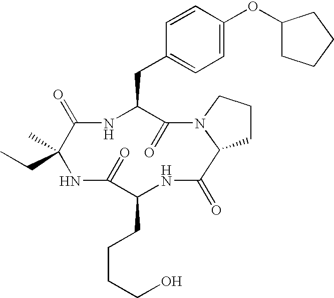 | 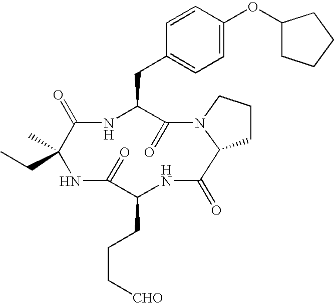 |
| Compound (410) | Compound (411) |
| 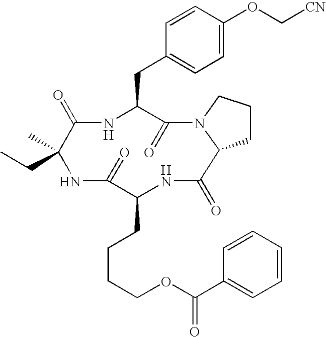 | 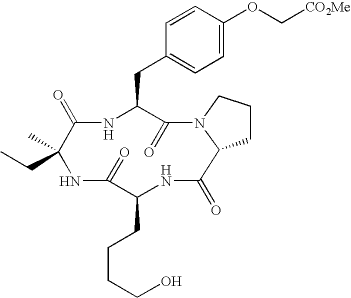 |
| Compound (412) | Compound (413) |
| 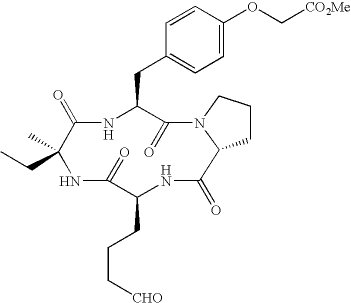 | 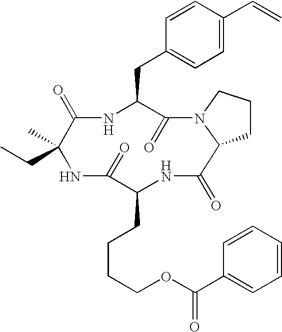 |

TABLE 2-52

| | |
|---|---|
| Compound (414) | Compound (415) |
| 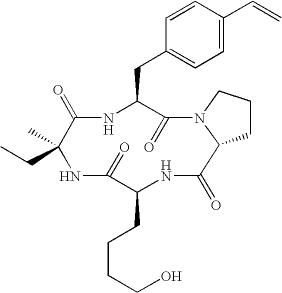 | 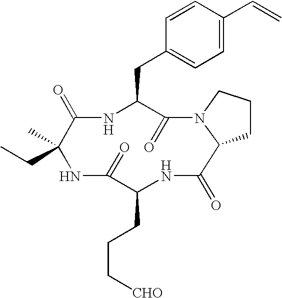 |
| Compound (416) | Compound (417) |
| 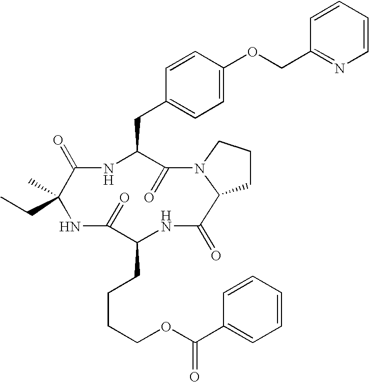 | 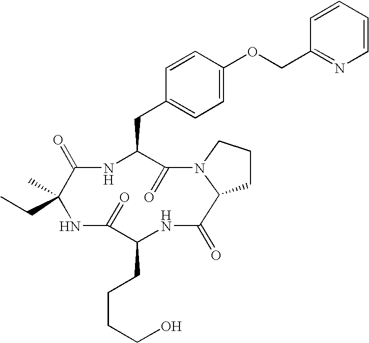 |
| Compound (418) | Compound (419) |
| 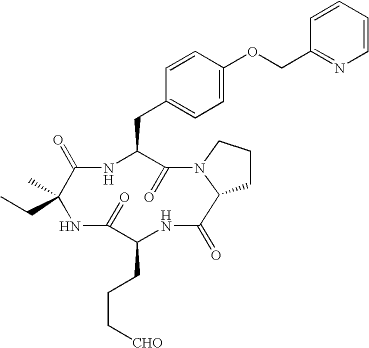 | 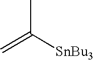 |
| Compound (420) | Compound (421) |
| 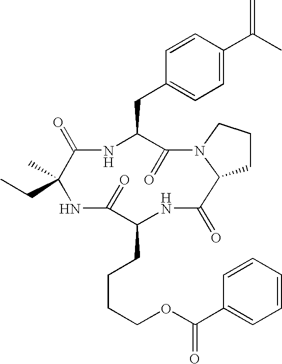 | 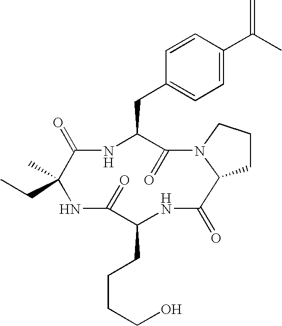 |

TABLE 2-53

| | |
|---|---|
| Compound (422) | Compound (423) |
| 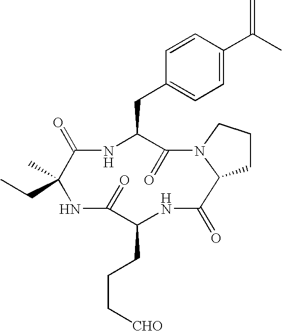 | 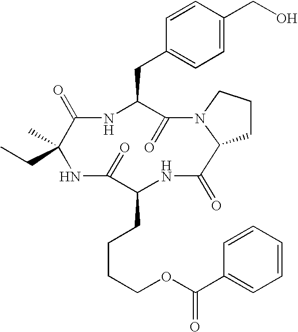 |
| Compound (424) | Compound (425) |
| 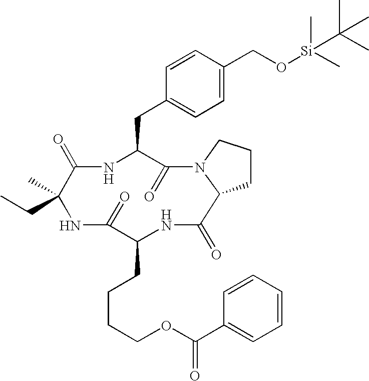 | 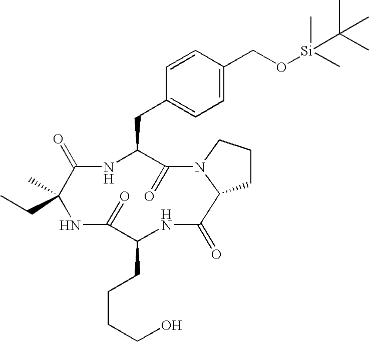 |
| Compound (426) | Compound (427) |
| 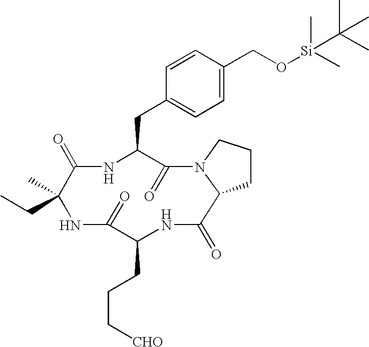 | 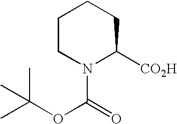 |
| Compound (428) | Compound (429) |
| 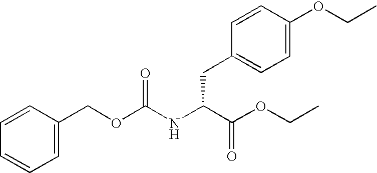 | 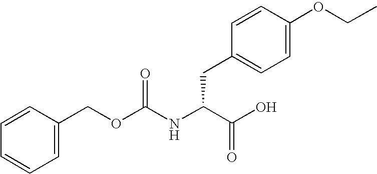 |

TABLE 2-54

| | |
|---|---|
| Compound (430) | Compound (431) |
| 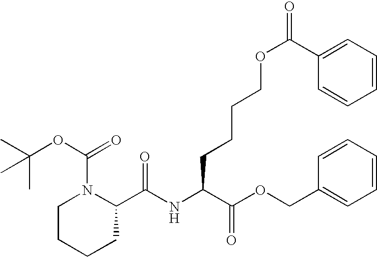 | 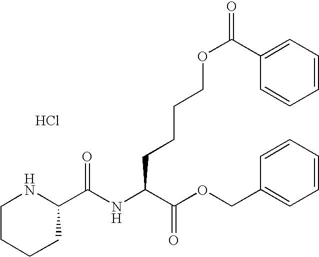 |
| Compound (432) | Compound (433) |
| 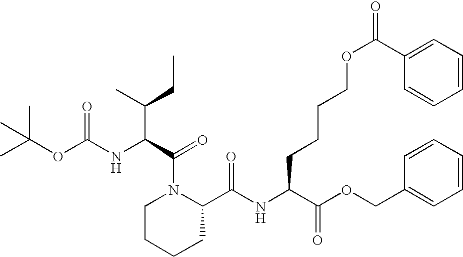 | 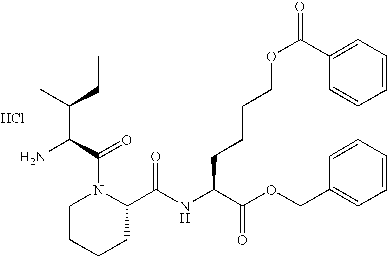 |
| Compound (434) | Compound (435) |
| 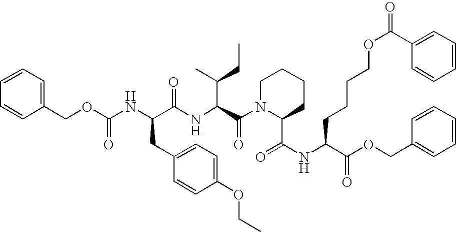 | 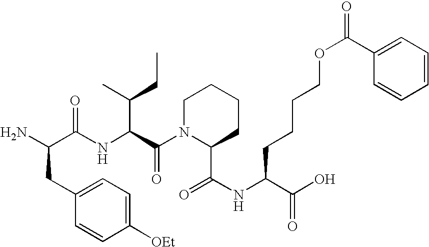 |
| Compound (436) | Compound (437) |
| 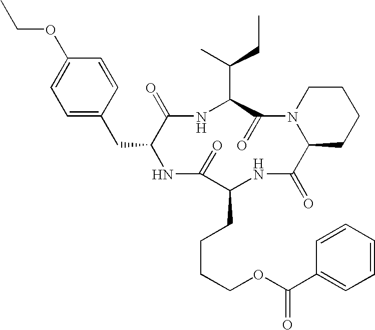 | 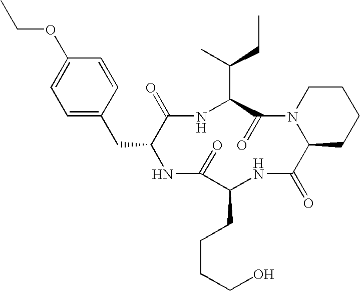 |

TABLE 2-55

| | |
|---|---|
| Compound (438) | Compound (439) |
| 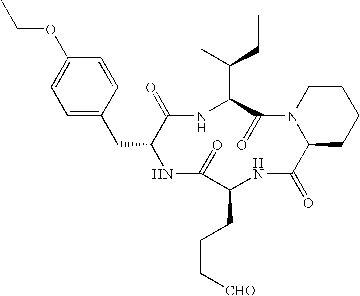 | 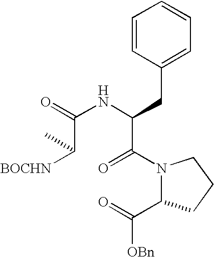 |
| Compound (440) | Compound (441) |
| 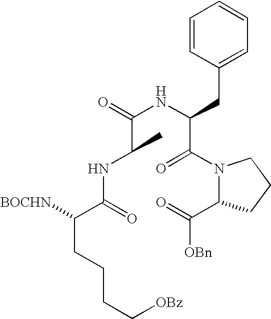 | 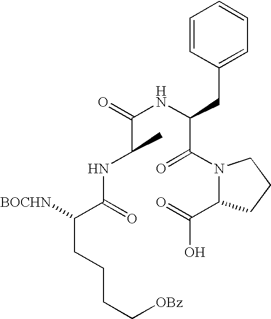 |
| Compound (442) | Compound (443) |
| 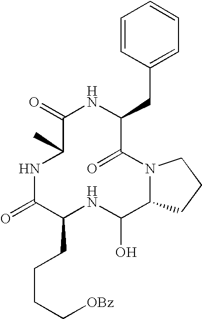 | 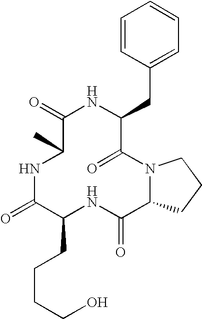 |
| Compound (444) | Compound (445) |
| 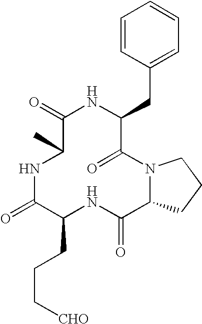 | 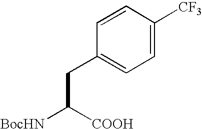 |

TABLE 2-56

| | |
|---|---|
| Compound (446) | Compound (447) |
| 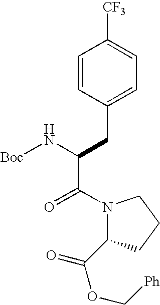 | 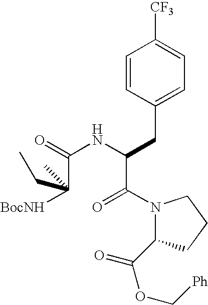 |
| Compound (448) | Compound (449) |
| 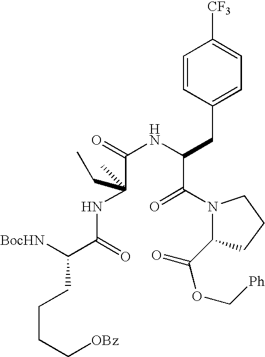 | 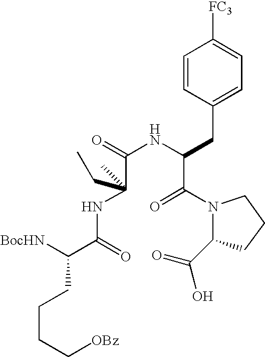 |
| Compound (450) | Compound (451) |
| 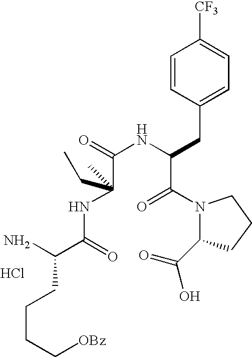 | 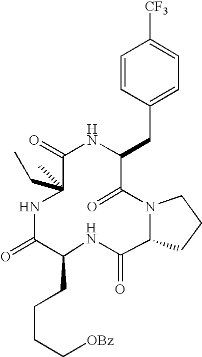 |
| Compound (452) | Compound (453) |
| 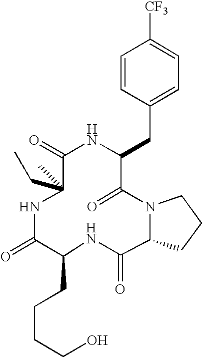 | 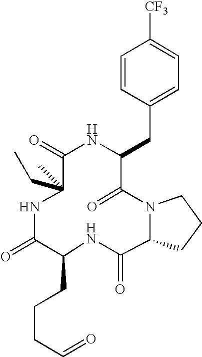 |

TABLE 2-57

| | |
|---|---|
| Compound (454) | Compound (455) |
| 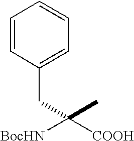 | 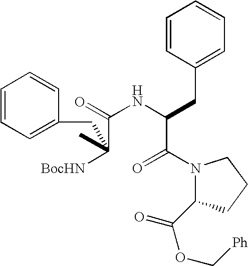 |
| Compound (456) | Compound (457) |
| 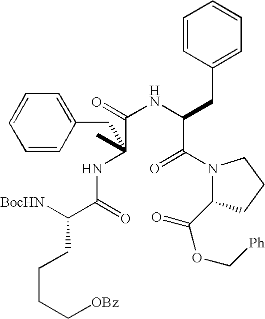 | 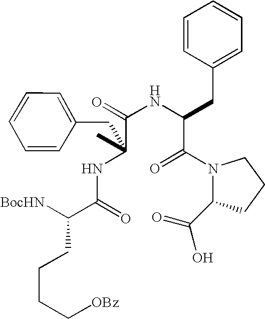 |
| Compound (458) | Compound (459) |
| 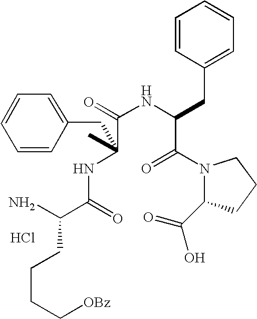 | 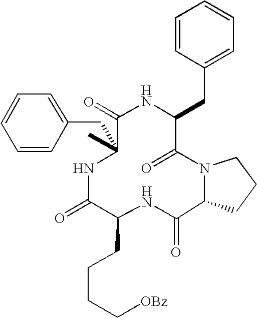 |
| Compound (460) | Compound (461) |
| 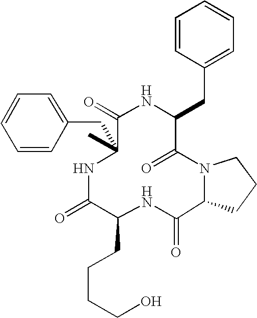 | 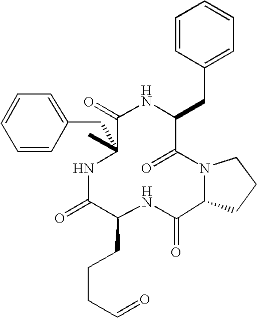 |

TABLE 2-58

| | |
|---|---|
| Compound (462) | Compound (463) |
| 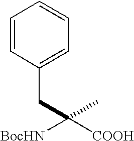 | 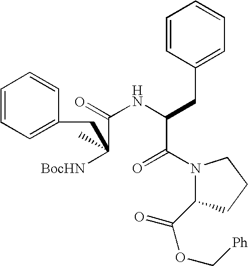 |
| Compound (464) | Compound (465) |
| 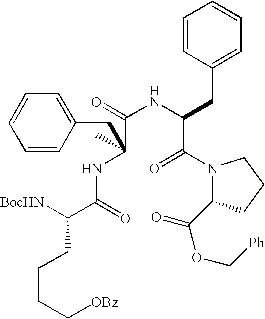 | 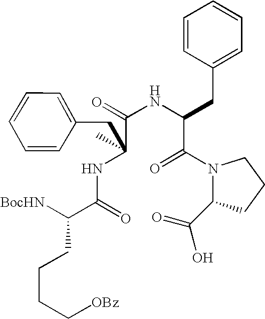 |
| Compound (466) | Compound (467) |
| 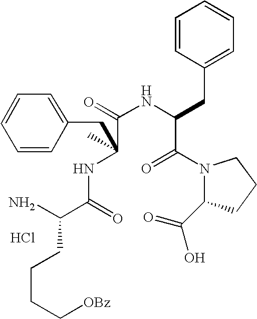 | 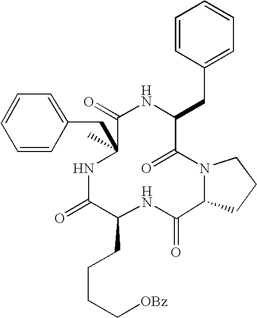 |
| Compound (468) | Compound (469) |
| 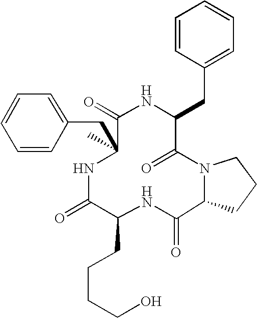 | 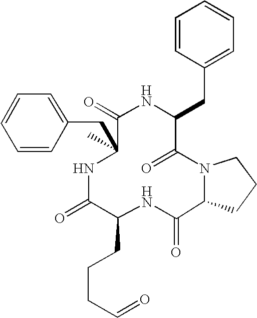 |

TABLE 2-59

| | |
|---|---|
| Compound (470) | Compound (471) |
| 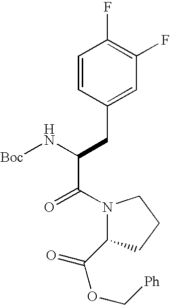 | 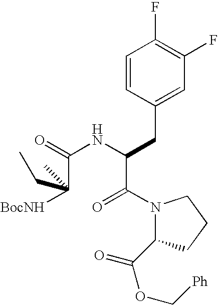 |
| Compound (472) | Compound (473) |
| 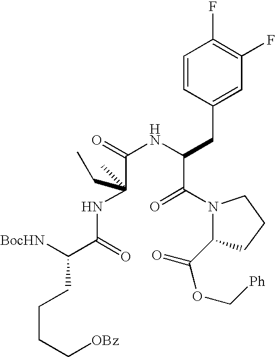 | 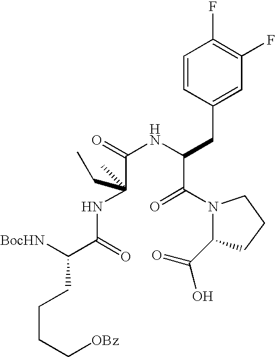 |
| Compound (474) | Compound (475) |
| 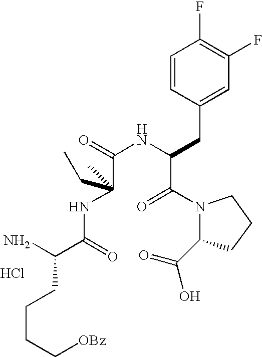 | 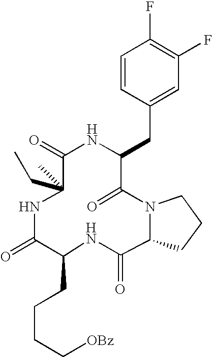 |
| Compound (476) | Compound (477) |
| 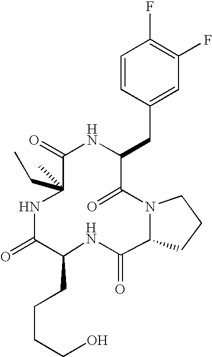 | 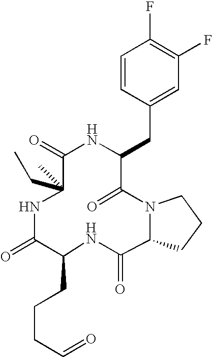 |

TABLE 2-60

| | |
|---|---|
| Compound (478) | Compound (479) |
| 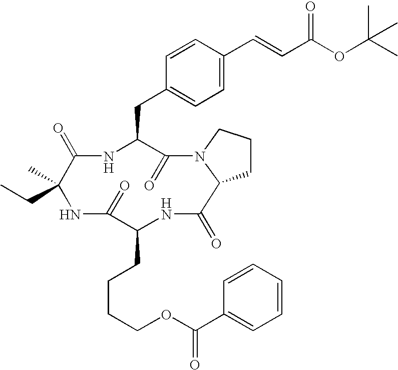 | 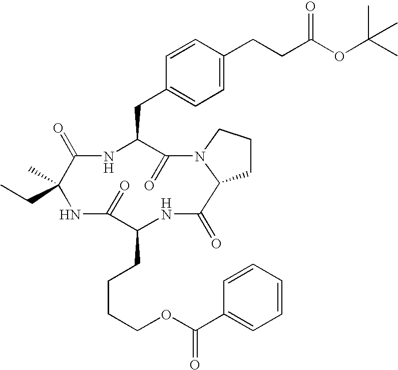 |
| Compound (480) | Compound (481) |
| 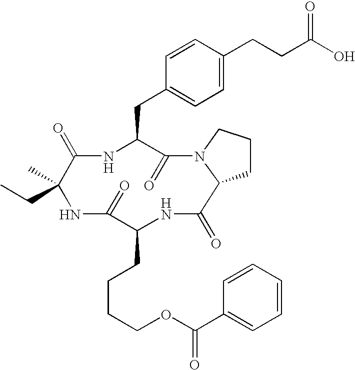 | 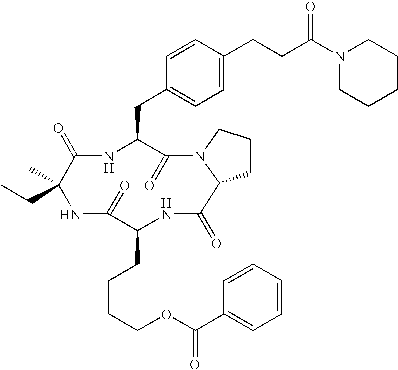 |
| Compound (482) | Compound (483) |
| 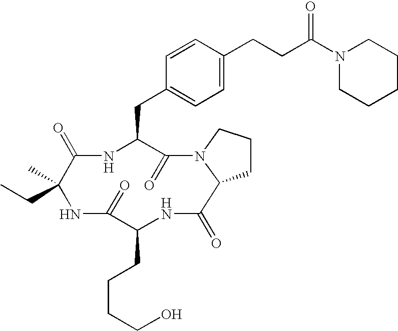 | 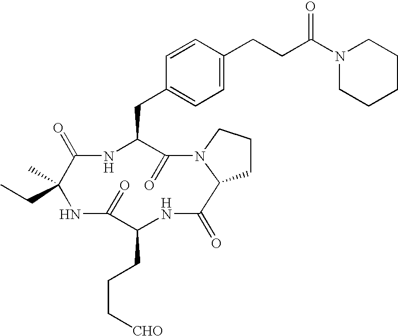 |
| Compound (484) | Compound (485) |
| 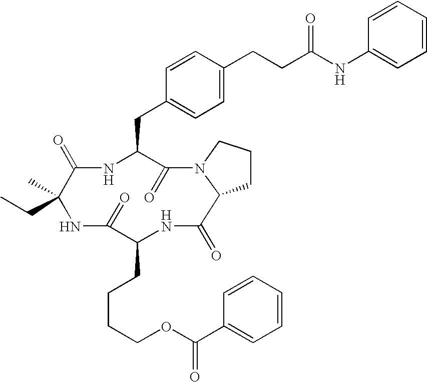 | 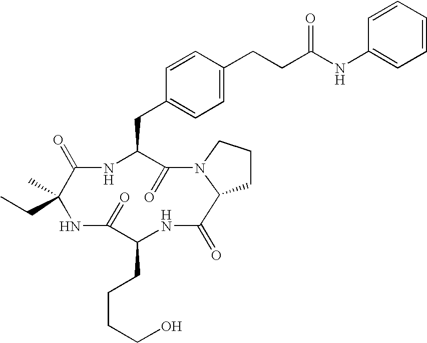 |

TABLE 2-61

| | |
|---|---|
| Compound (486) | Compound (487) |
| 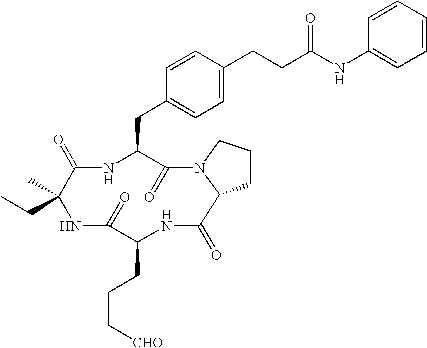 | 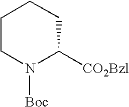 |
| Compound (488) | Compound (489) |
| 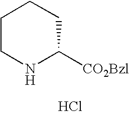 | 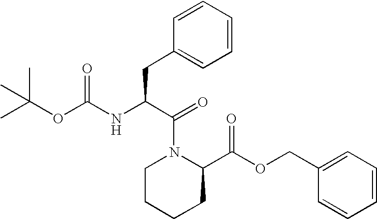 |
| Compound (490) | Compound (491) |
| 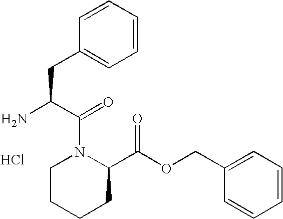 | 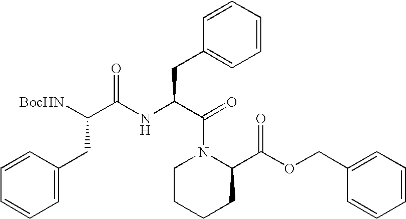 |
| Compound (492) | Compound (493) |
| 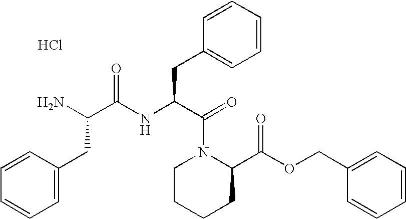 | 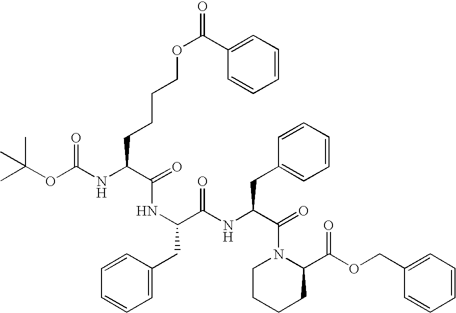 |

TABLE 2-62

| | |
|---|---|
| Compound (494) | Compound (495) |
| 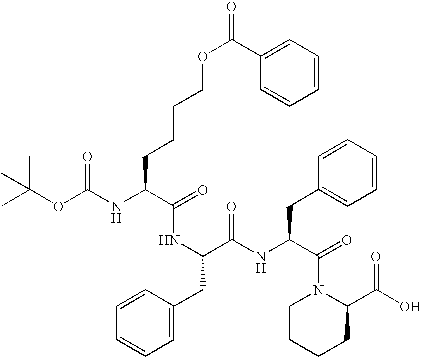 | 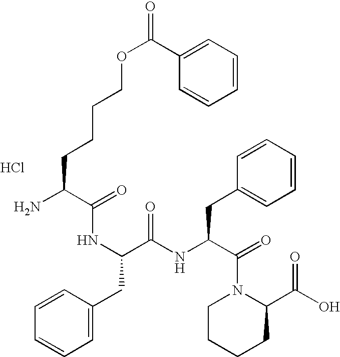 |
| Compound (496) | Compound (497) |
| 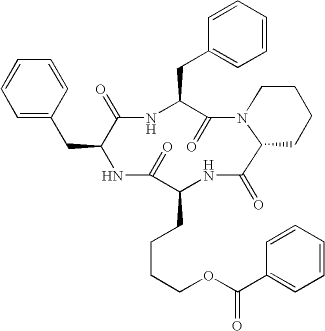 | 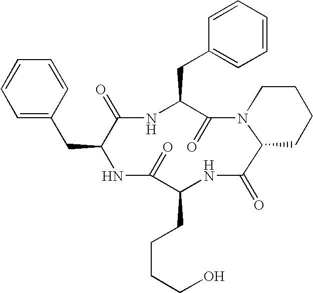 |
| Compound (498) | Compound (499) |
| 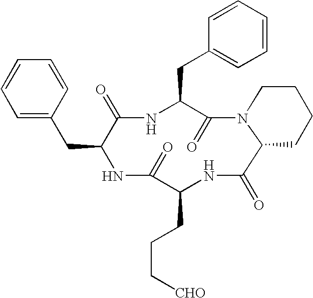 | 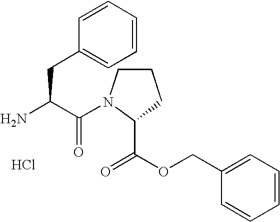 |
| Compound (500) | Compound (501) |
| 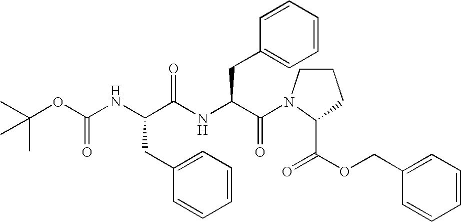 | 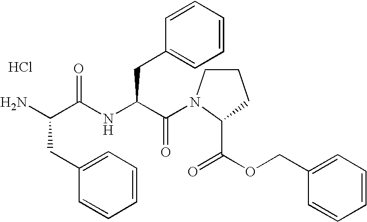 |

TABLE 2-63

| | |
|---|---|
| Compound (502) | Compound (503) |
| 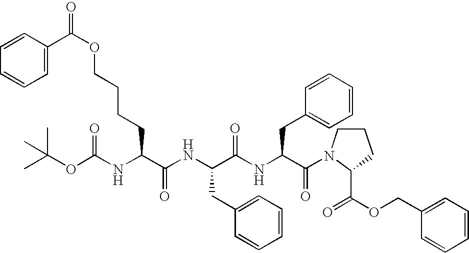 | 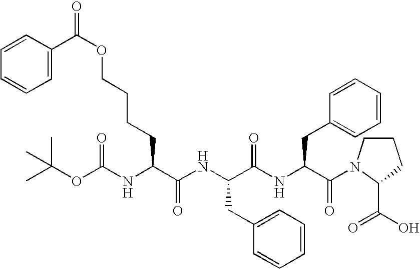 |
| Compound (504) | Compound (505) |
| 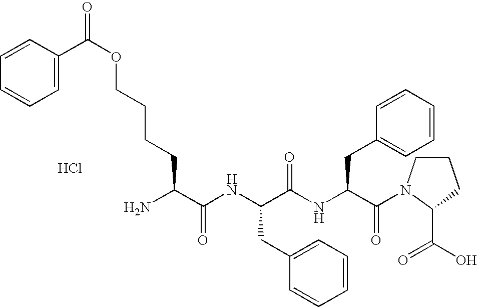 | 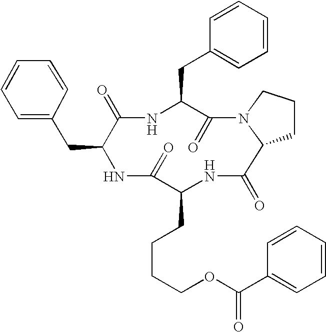 |
| Compound (506) | Compound (507) |
| 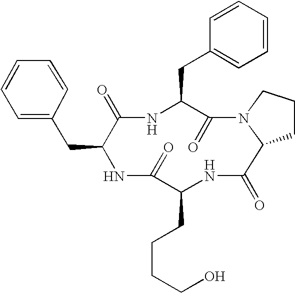 | 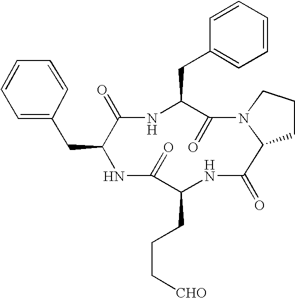 |
| Compound (508) | Compound (509) |
| 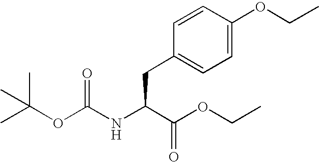 | 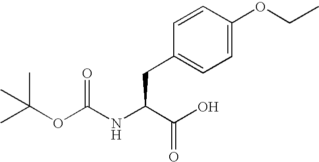 |

TABLE 2-64

| | |
|---|---|
| Compound (510) | Compound (511) |
| 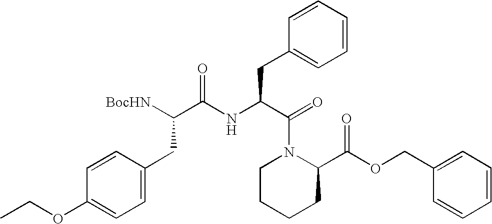 | 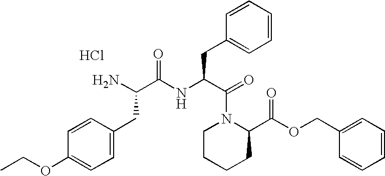 |
| Compound (512) | Compound (513) |
| 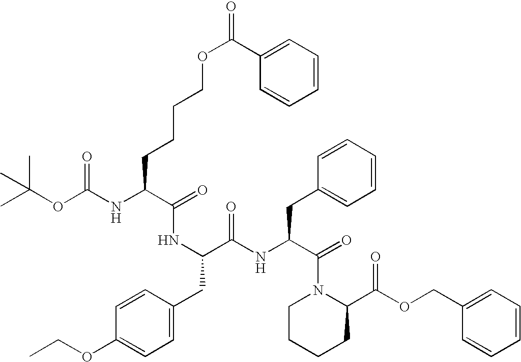 | 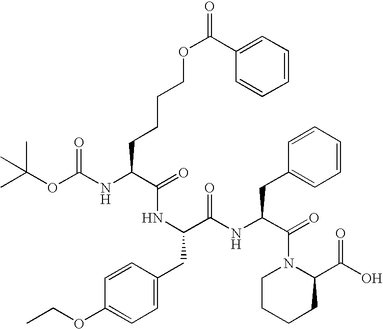 |
| Compound (514) | Compound (515) |
| 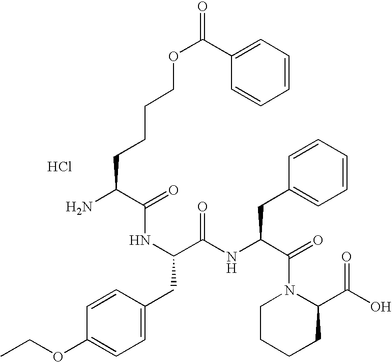 | 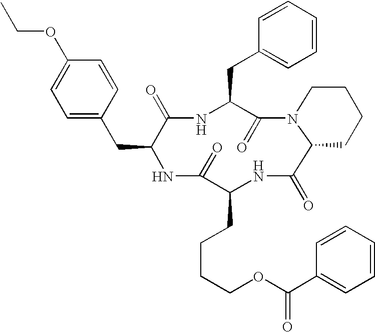 |
| Compound (516) | Compound (517) |
| 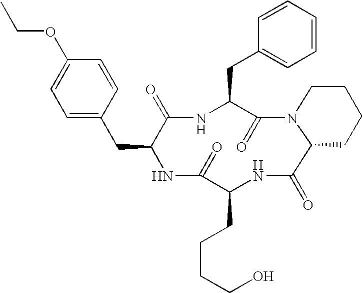 | 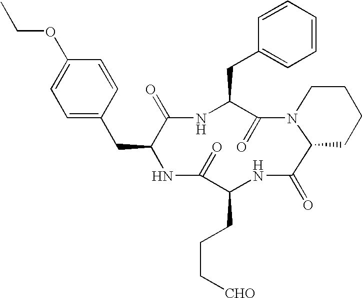 |

TABLE 2-65

| | |
|---|---|
| Compound (518) | Compound (519) |
| 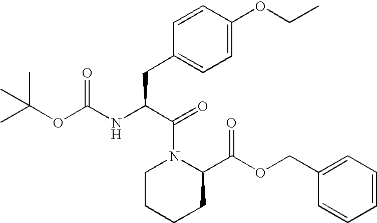 | 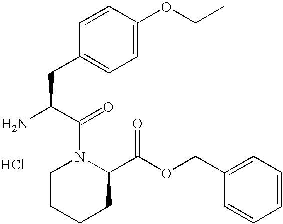 |
| Compound (520) | Compound (521) |
| 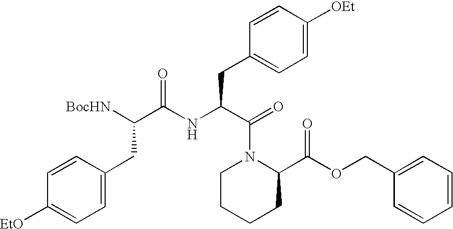 | 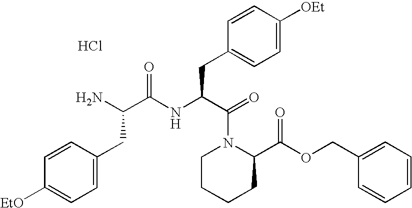 |
| Compound (522) | Compound (523) |
| 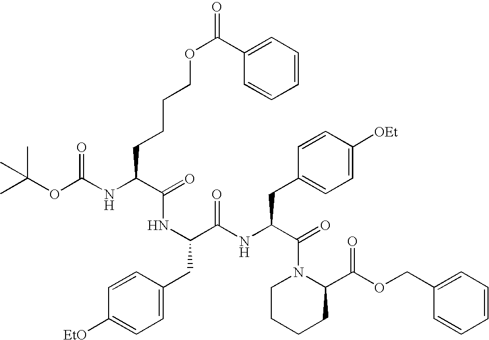 | 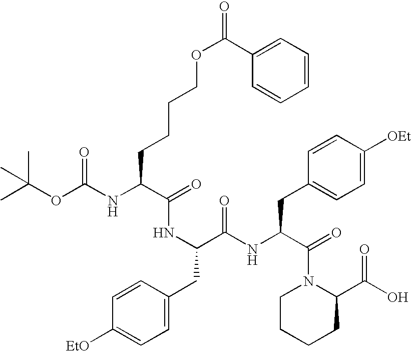 |
| Compound (524) | Compound (525) |
| 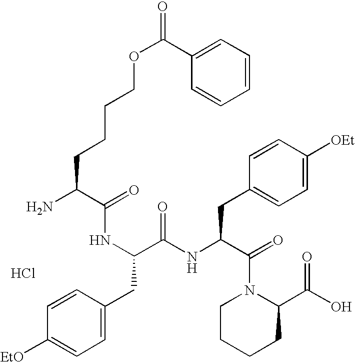 | 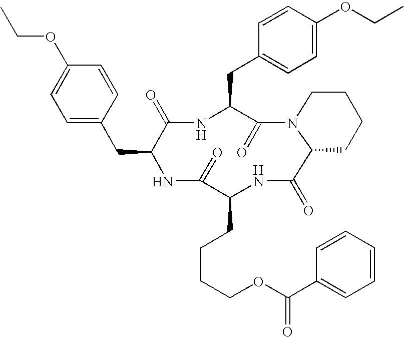 |

TABLE 2-66

| | |
|---|---|
| Compound (526) | Compound (527) |
| 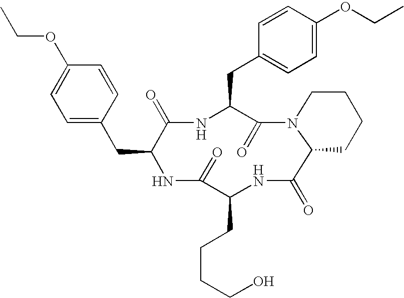 | 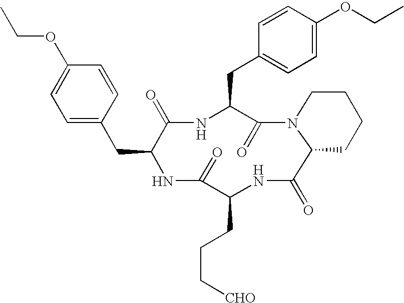 |
| Compound (528) | Compound (529) |
| 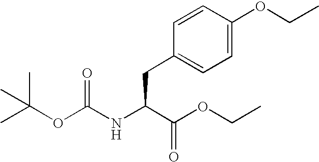 | 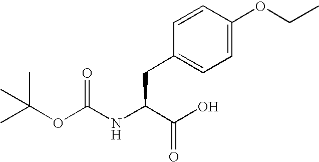 |
| Compound (530) | Compound (531) |
| 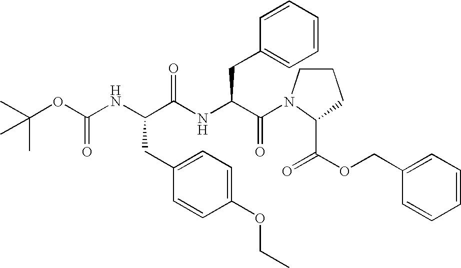 | 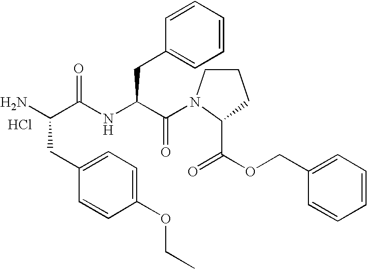 |
| Compound (532) | Compound (533) |
| 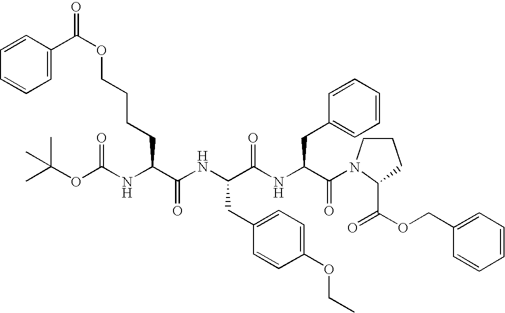 | 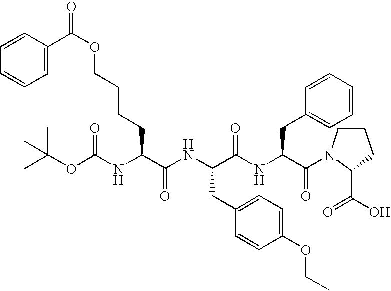 |

TABLE 2-67

| | |
|---|---|
| Compound (534) | Compound (535) |
| 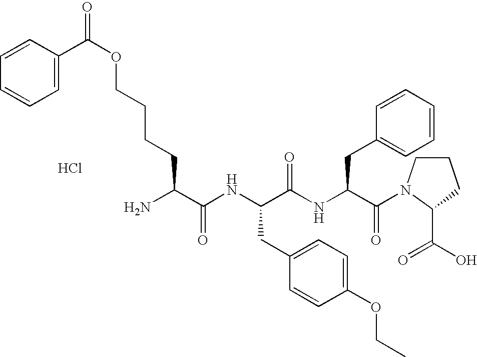 | 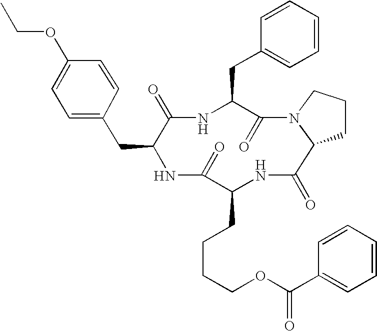 |
| Compound (536) | Compound (537) |
| 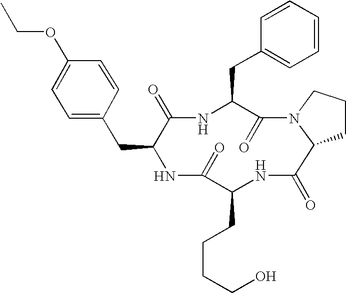 | 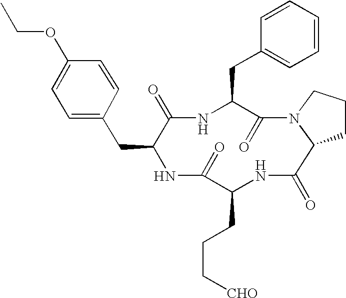 |
| Compound (538) | Compound (539) |
| 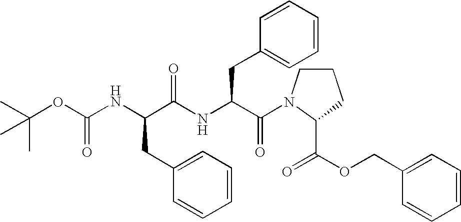 | 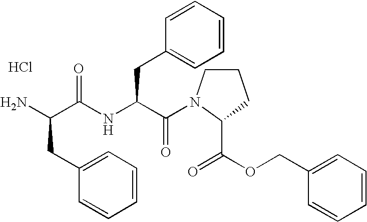 |
| Compound (540) | Compound (541) |
| 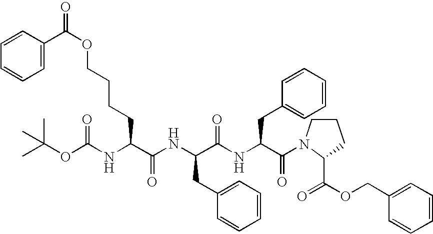 | 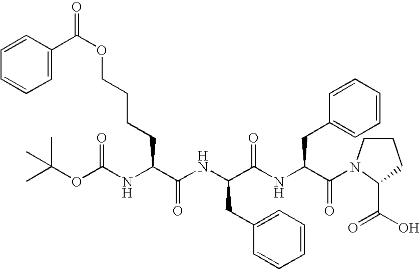 |

TABLE 2-68

| | |
|---|---|
| Compound (542) | Compound (543) |
| 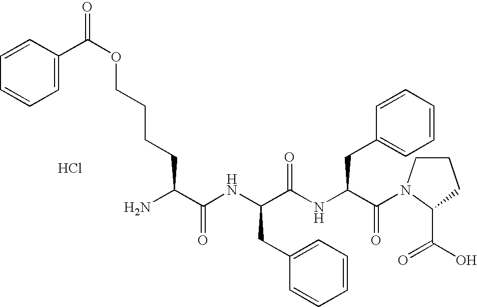 | 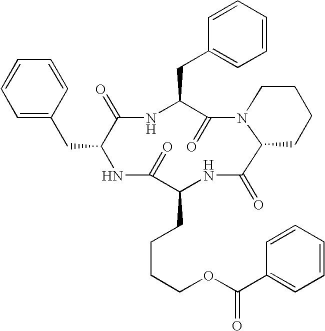 |
| Compound (544) | Compound (545) |
| 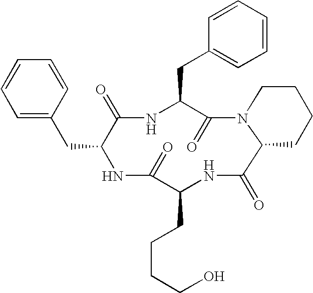 | 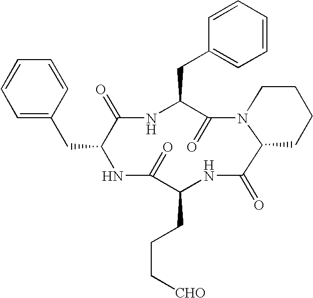 |
| Compound (546) | Compound (547) |
| 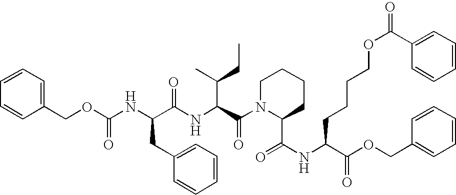 | 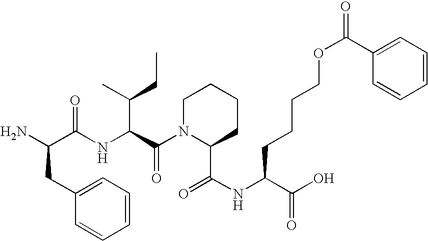 |
| Compound (548) | Compound (549) |
| 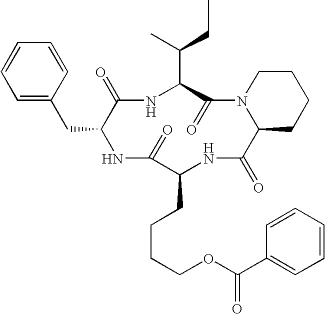 | 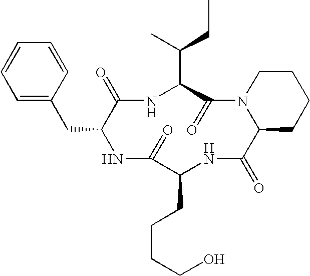 |

TABLE 2-69

| | |
|---|---|
| Compound (550) | Compound (551) |
| 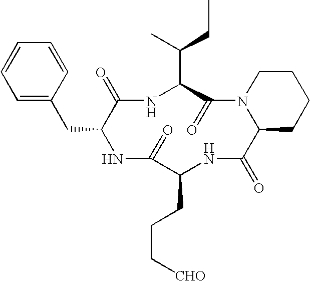 | 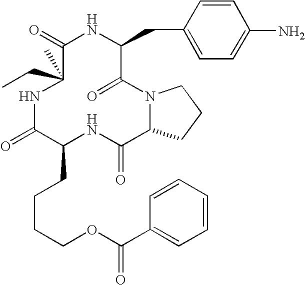 |
| Compound (552) | Compound (553) |
| 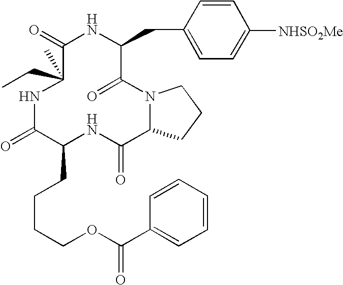 | 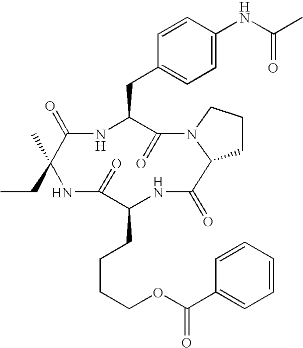 |

TABLE 2-70

| | |
|---|---|
| Compound E1 | Compound E2 |
| 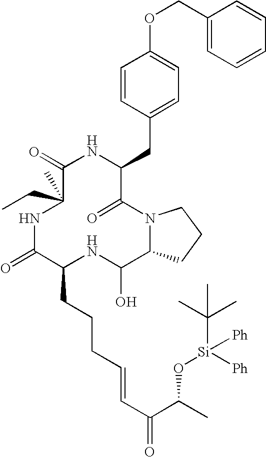 | 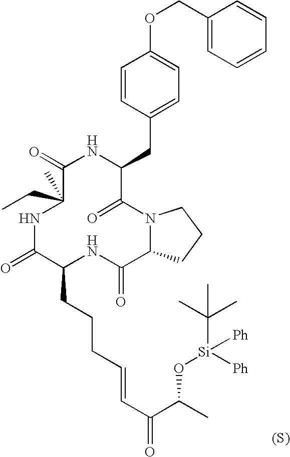 |
| Compound E3 | Compound E4 |
| 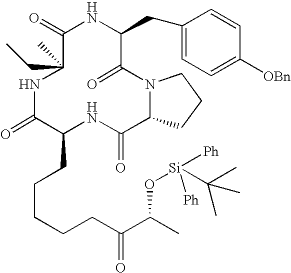 | 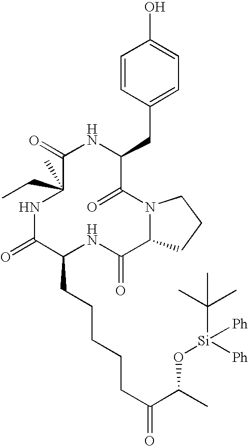 |
| Compound E5 | Compound E6 |
| 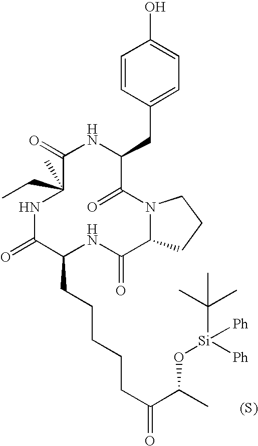 | 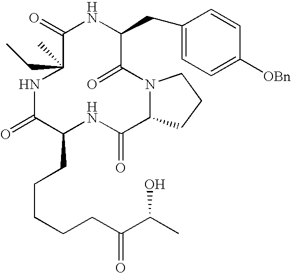 |
| Compound E7 | Compound E8 |
| 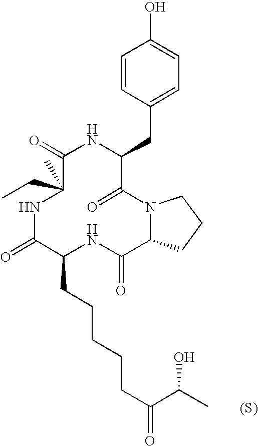 | 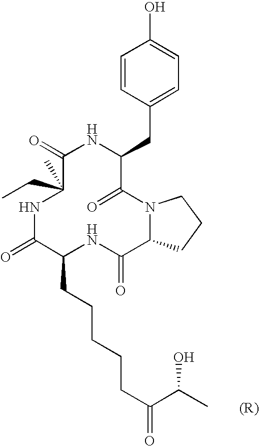 |

TABLE 2-71

| | |
|---|---|
| Compound E9 | Compound E10 |
| 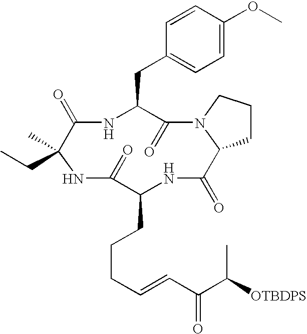 | 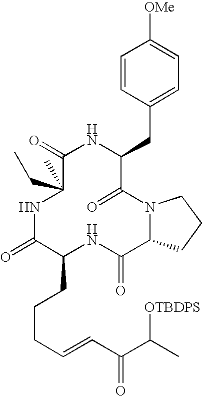 |
| Compound E11 | Compound E12 |
| 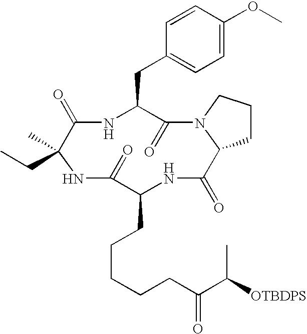 | 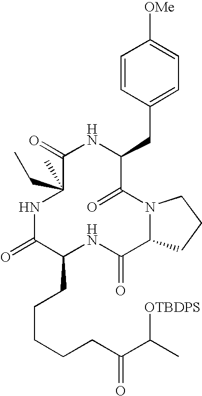 |
| Compound E13 | Compound E14 |
| 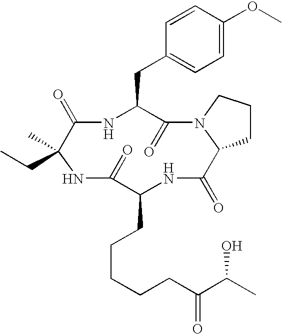 | 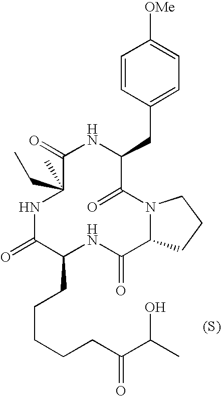 |
| Compound E15 | Compound E16 |
| 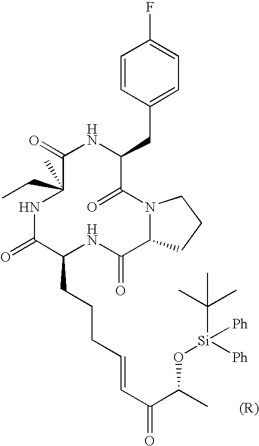 | 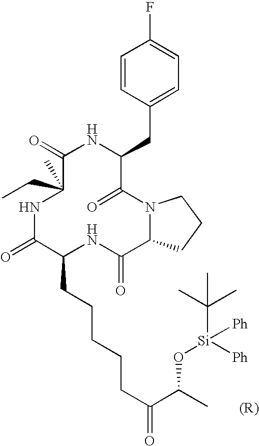 |

TABLE 2-72

| | |
|---|---|
| Compound E17 | Compound E18 |
| 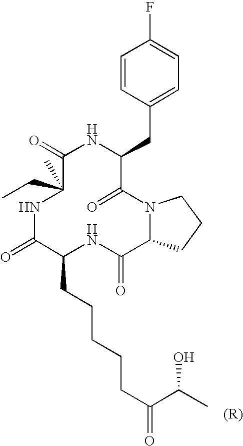 | 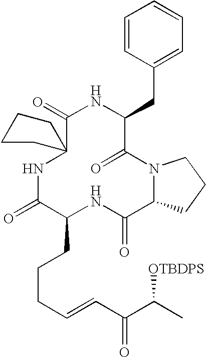 |
| Compound E19 | Compound E20 |
| 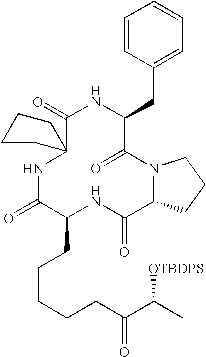 | 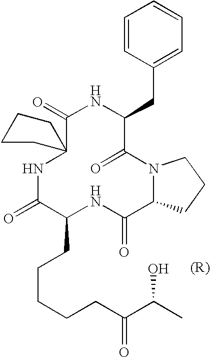 |
| Compound E21 | Compound E22 |
| 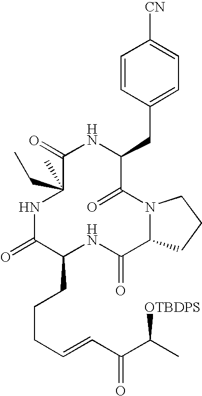 | 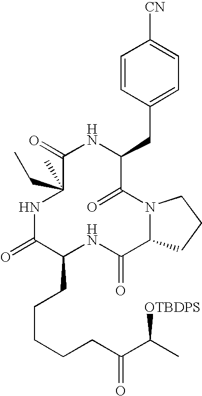 |
| Compound E23 | Compound E24 |
| 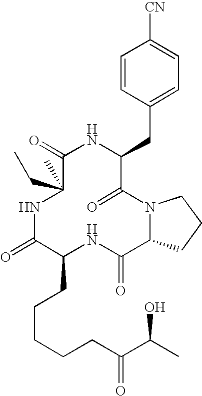 | 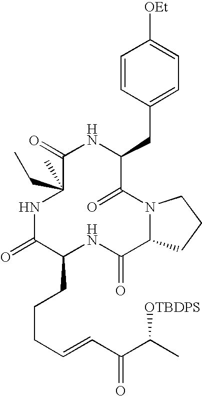 |

TABLE 2-73

| | |
|---|---|
| Compound E25 | Compound E26 |
| 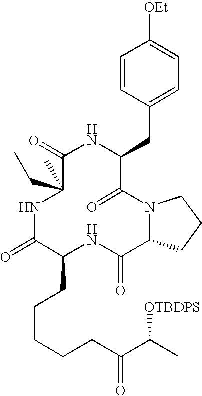 | 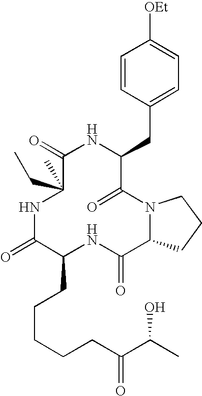 |
| Compound E27 | Compound E28 |
| 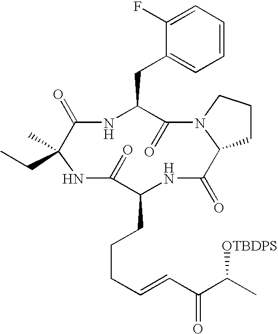 | 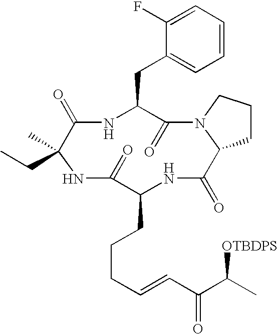 |
| Compound E29 | Compound E30 |
| 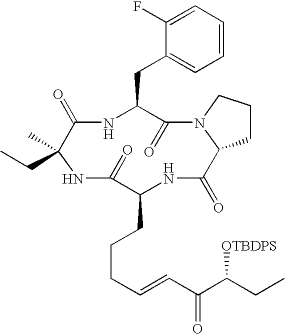 | 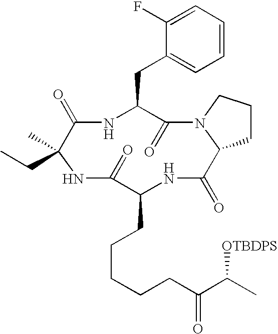 |
| Compound E31 | Compound E32 |
| 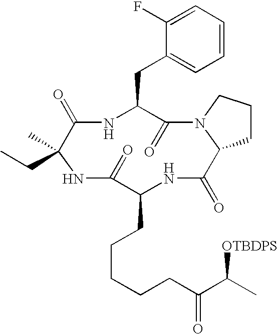 | 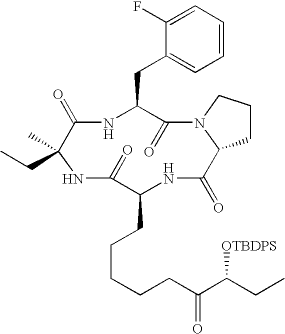 |

TABLE 2-74

| | |
|---|---|
| Compound E33 | Compound E34 |
| 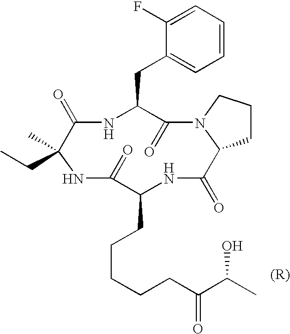 | 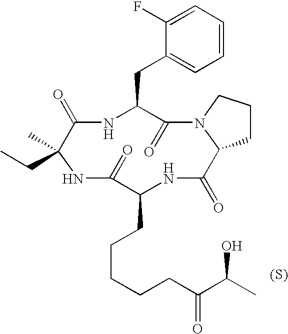 |
| Compound E35 | Compound E36 |
| 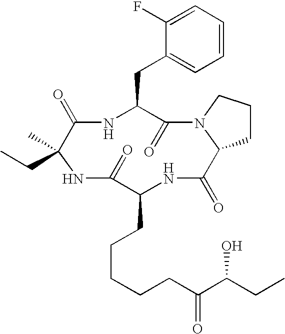 | 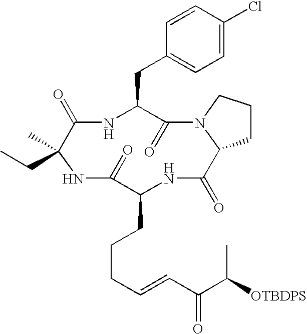 |
| Compound E37 | Compound E38 |
| 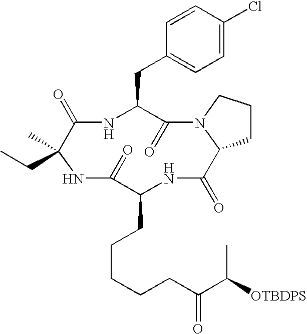 | 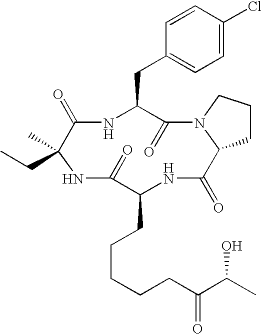 |
| Compound E39 | Compound E40 |
| 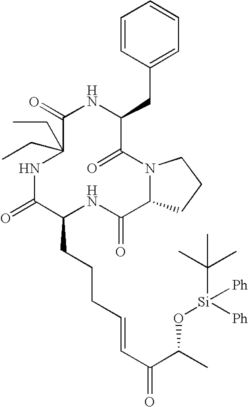 | 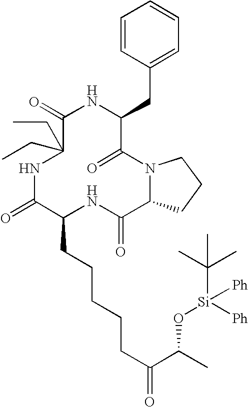 |

TABLE 2-75

| | |
|---|---|
| Compound E41 | Compound E42 |
| 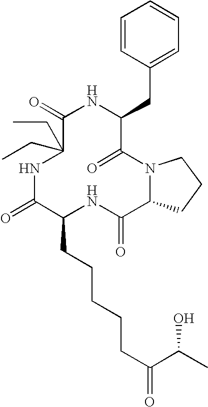 | 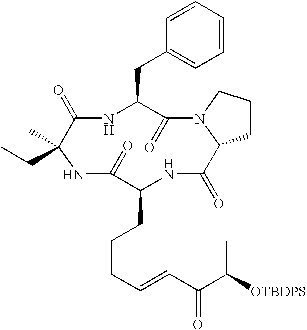 |
| Compound E43 | Compound E44 |
| 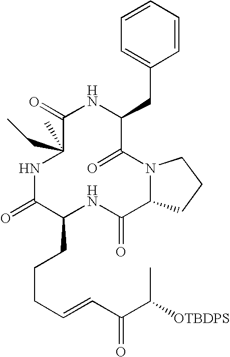 | 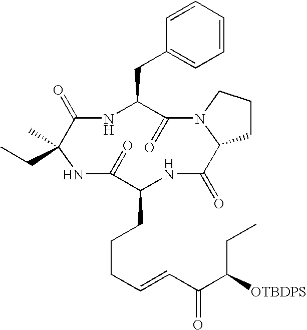 |
| Compound E45 | Compound E46 |
| 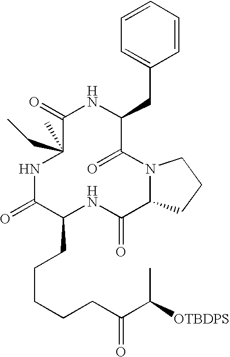 | 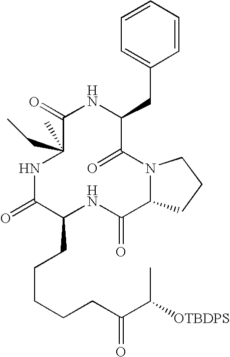 |
| Compound E47 | Compound E48 |
| 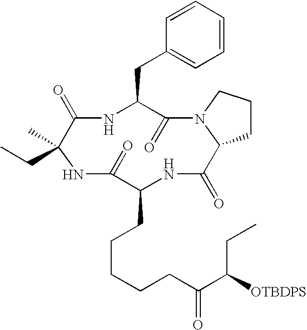 | 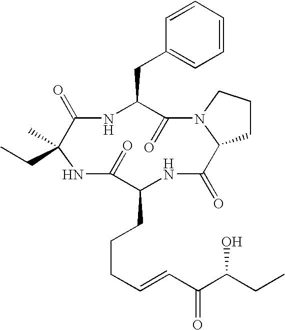 |

TABLE 2-76

| | |
|---|---|
| Compound E49 | Compound E50 |
| 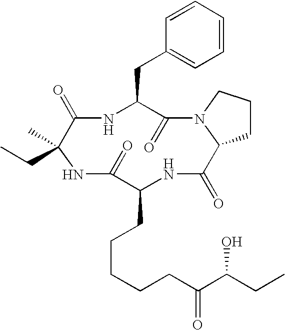 | 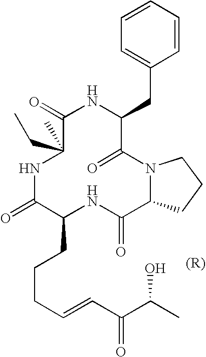 |
| Compound E51 | Compound E52 |
| 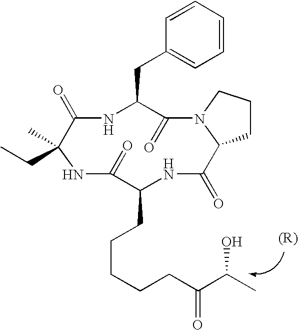 | 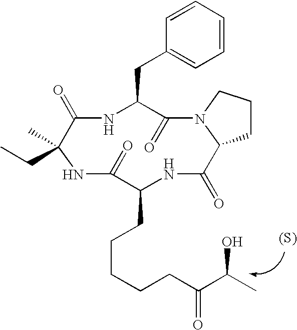 |
| Compound E53 | Compound E54 |
| 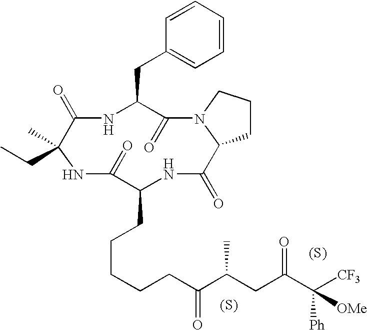 | 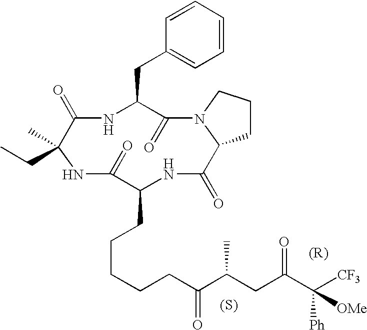 |
| Compound E55 | Compound E56 |
| 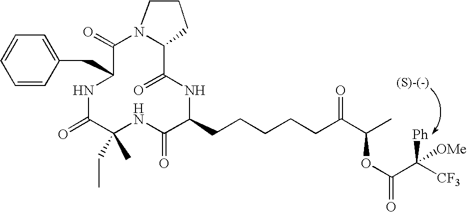 | 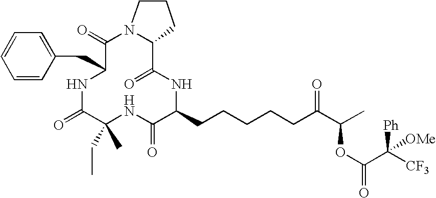 |

TABLE 2-77

| | |
|---|---|
| Compound E57 | Compound E58 |
| 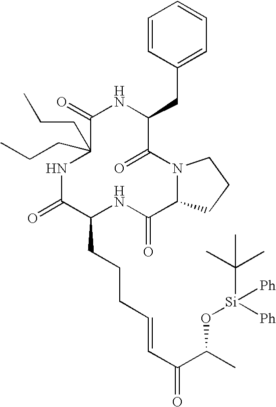 | 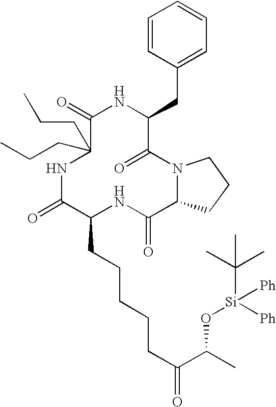 |
| Compound E59 | Compound E60 |
| 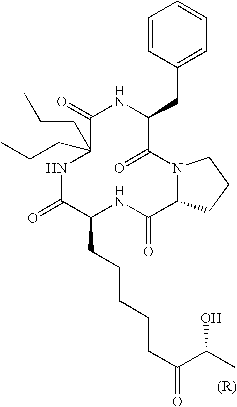 | 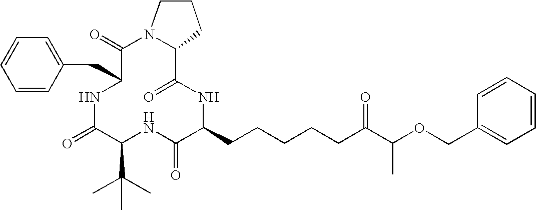 |
| Compound E61 | Compound E62 |
| 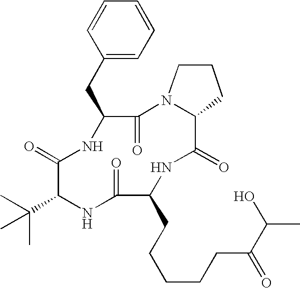 | 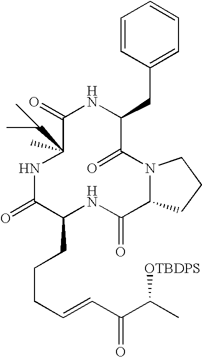 |
| Compound E63 | Compound E64 |
| 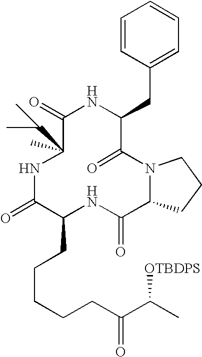 | 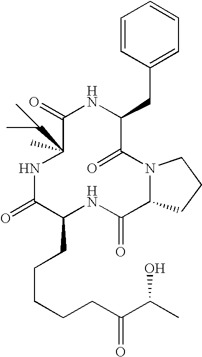 |

TABLE 2-78

| | |
|---|---|
| Compound E65 | Compound E66 |
| 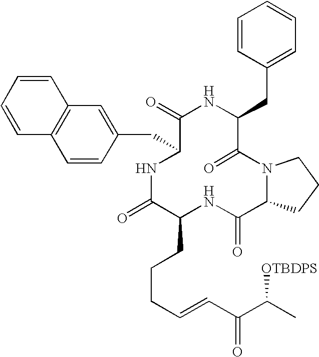 | 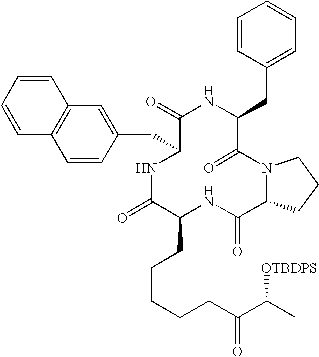 |
| Compound E67 | Compound E68 |
| 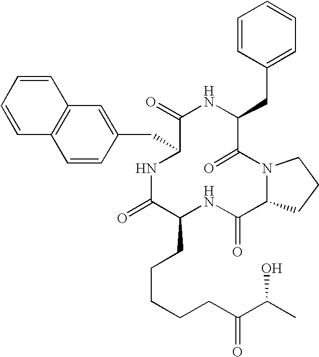 | 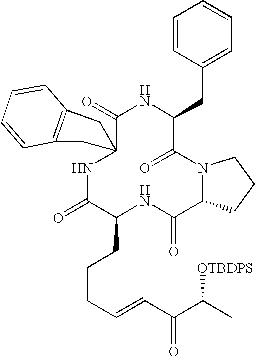 |
| Compound E69 | Compound E70 |
| 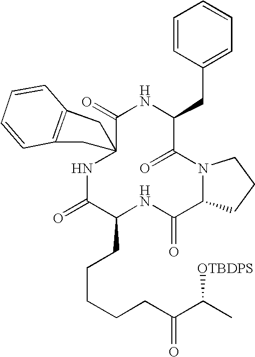 | 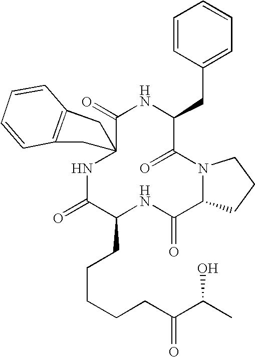 |
| Compound E71 | Compound E72 |
| 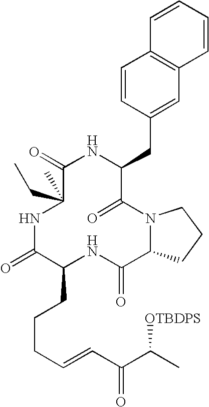 | 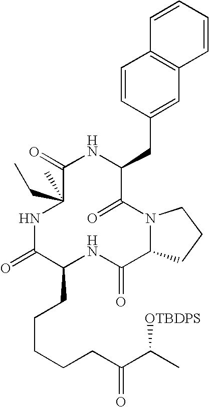 |

TABLE 2-79

| | |
|---|---|
| Compound E73 | Compound E74 |
| 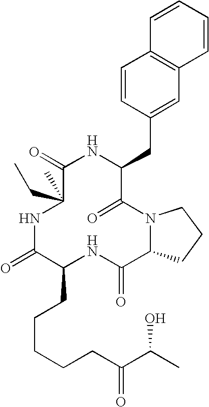 | 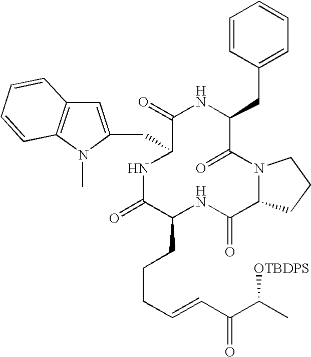 |
| Compound E75 | Compound E76 |
| 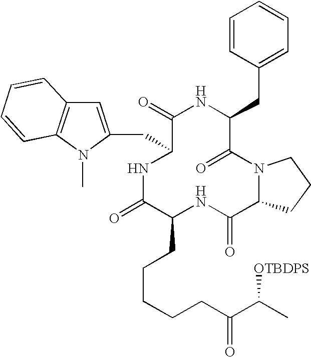 | 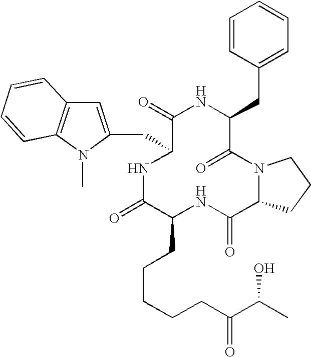 |
| Compound E77 | Compound E78 |
| 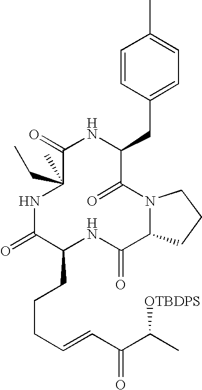 | 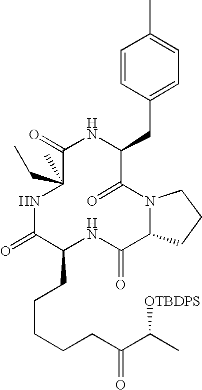 |
| Compound E79 | Compound E80 |
| 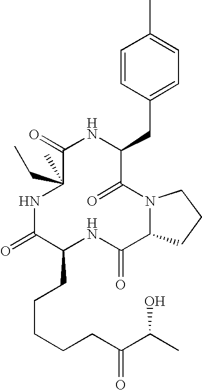 | 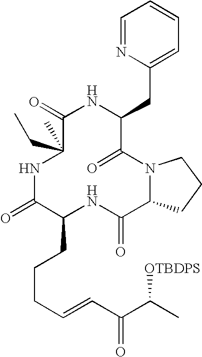 |

TABLE 2-80

| | |
|---|---|
| Compound E81 | Compound E82 |
| 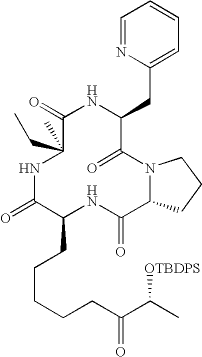 | 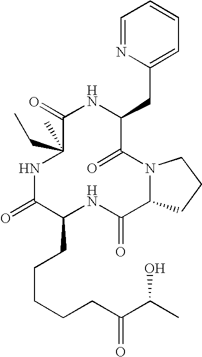 |
| Compound E83 | Compound E84 |
| 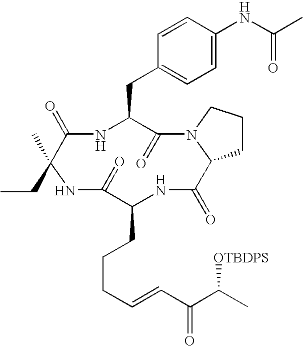 | 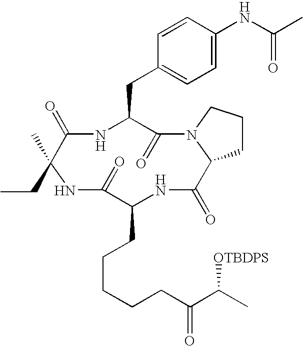 |
| Compound E85 | Compound E86 |
| 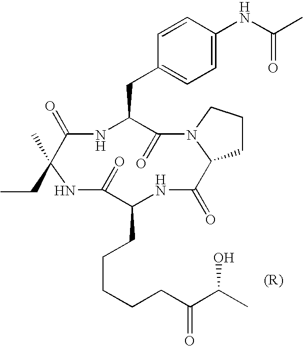 | 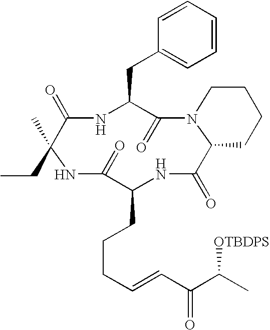 |
| Compound E87 | Compound E88 |
| 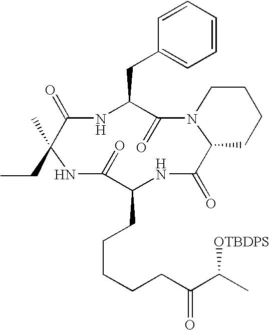 | 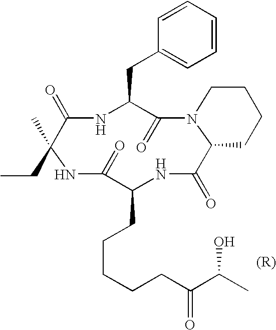 |

TABLE 2-81

| | |
|---|---|
| Compound E89 | Compound E90 |
| 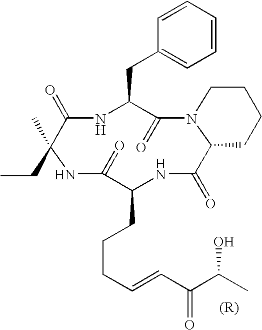 | 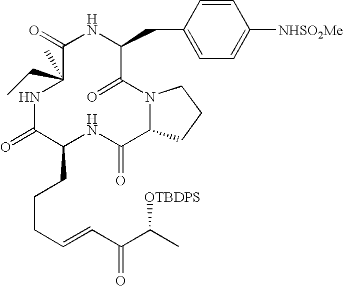 |
| Compound E91 | Compound E92 |
| 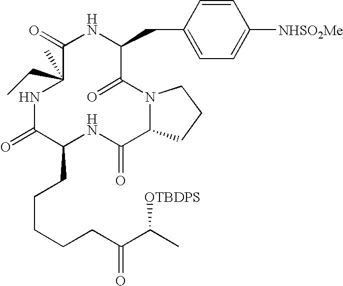 | 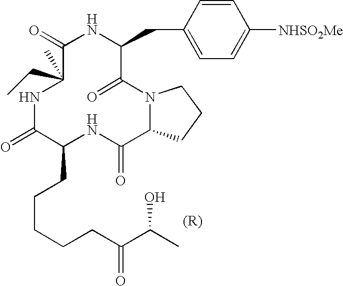 |
| Compound E93 | Compound E94 |
| 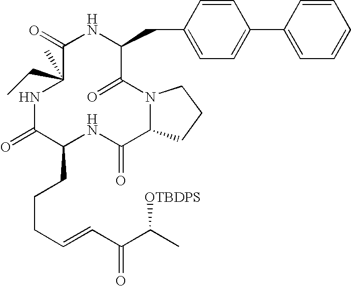 | 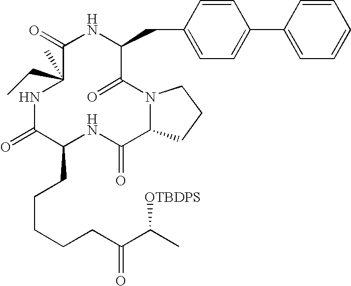 |
| Compound E95 | Compound E96 |
| 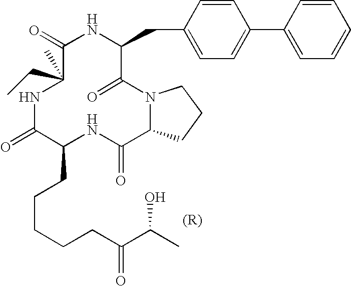 | 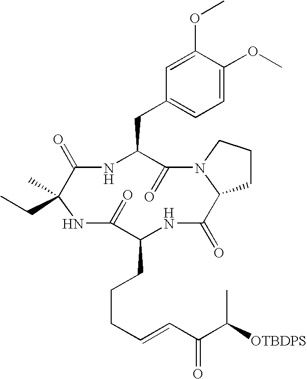 |

TABLE 2-82

| | |
|---|---|
| Compound E97 | Compound E98 |
| 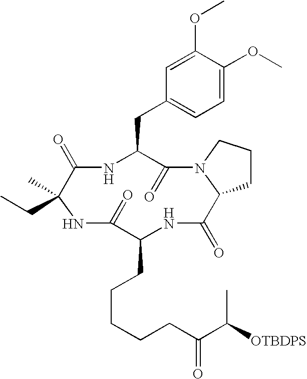 | 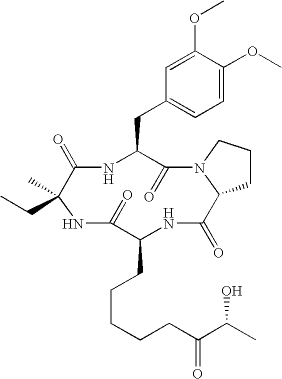 |
| Compound E99 | Compound E100 |
| 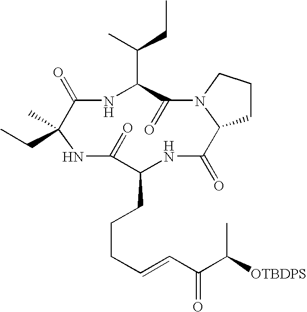 | 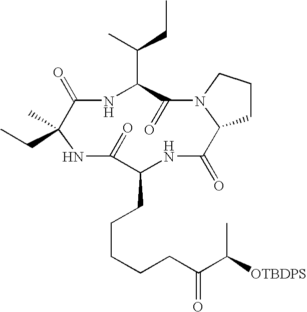 |
| Compound E101 | Compound E102 |
| 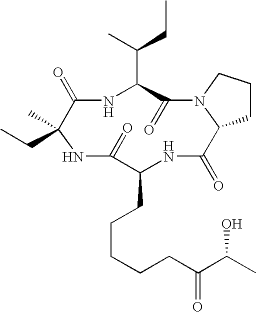 | 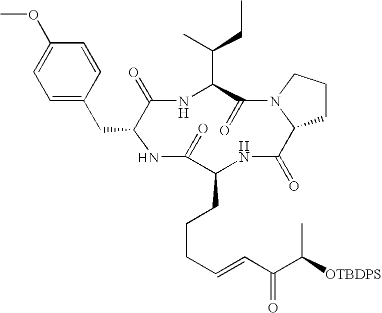 |
| Compound E103 | Compound E104 |
| 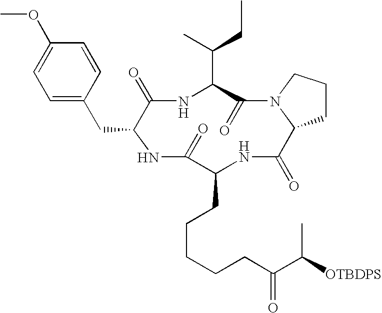 | 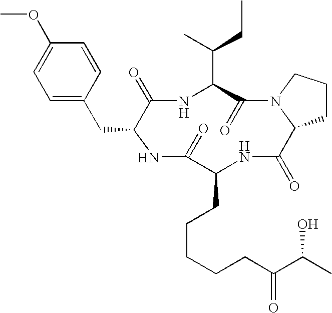 |

TABLE 2-83

| | |
|---|---|
| Compound E105 | Compound E106 |
| 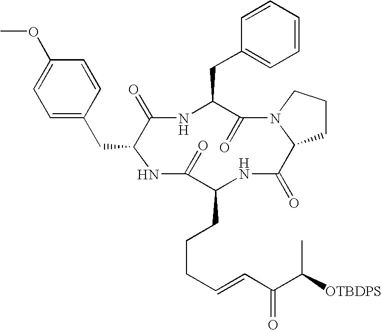 | 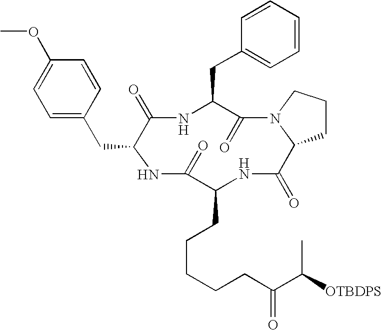 |
| Compound E107 | Compound E108 |
| 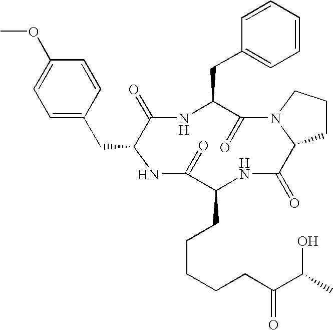 | 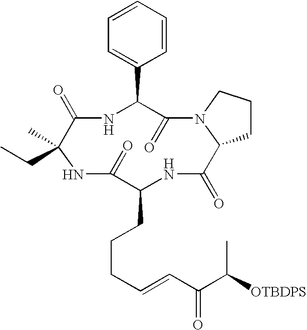 |
| Compound E109 | Compound E110 |
| 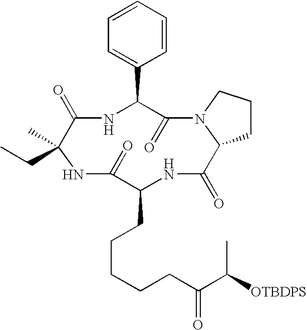 | 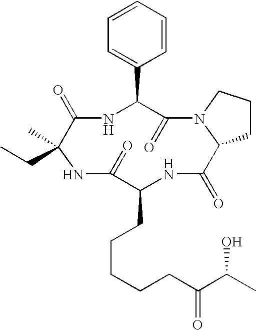 |
| Compound E111 | Compound E112 |
| 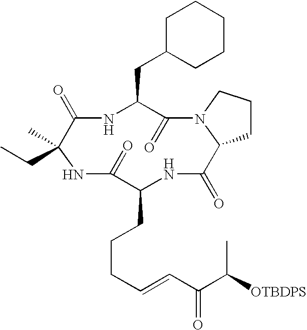 | 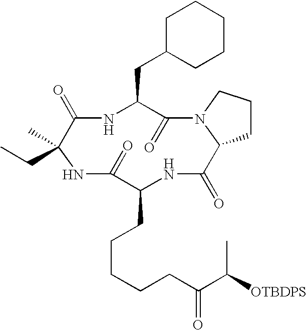 |

TABLE 2-84

| | |
|---|---|
| Compound E113 | Compound E114 |
| 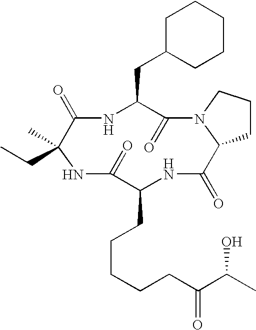 | 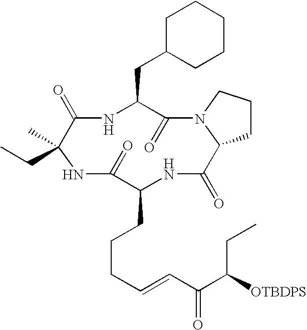 |
| Compound E115 | Compound E116 |
| 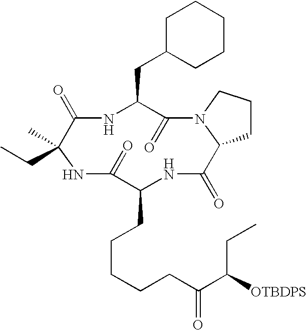 | 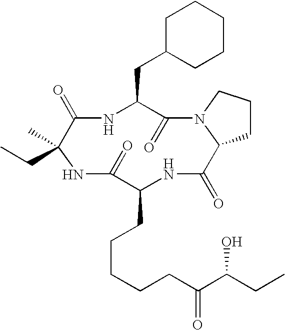 |
| Compound E117 | Compound E118 |
| 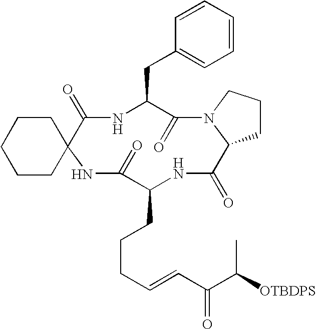 | 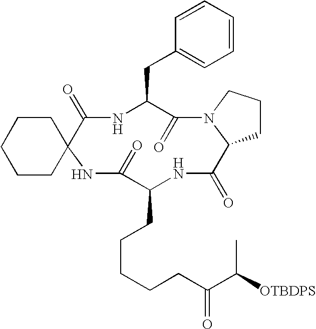 |
| Compound E119 | Compound E120 |
| 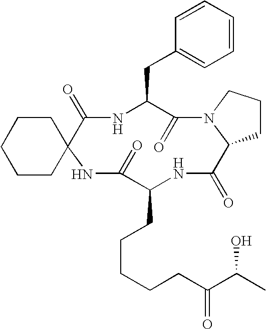 | 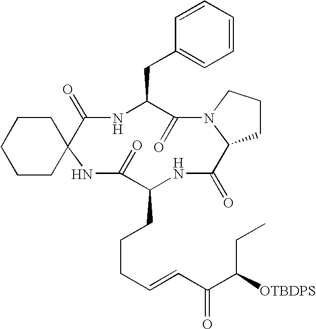 |

TABLE 2-85

| | |
|---|---|
| Compound E121 | Compound E122 |
| 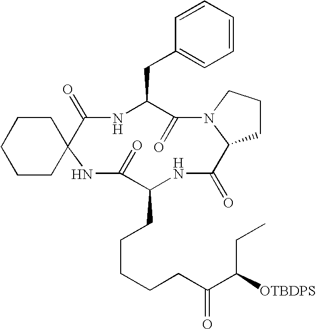 | 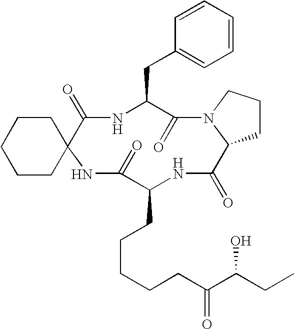 |
| Compound E123 | Compound E124 |
| 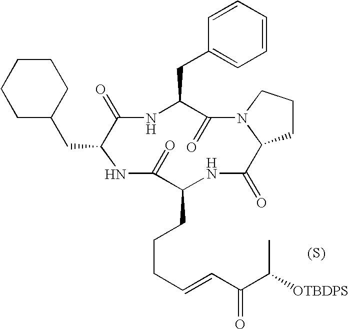 | 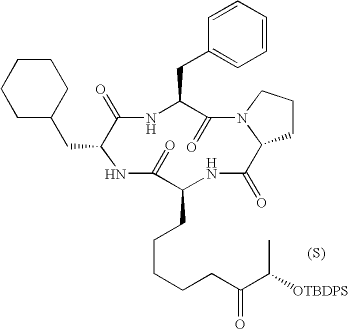 |
| Compound E125 | Compound E126 |
| 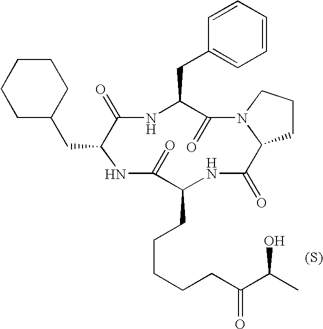 | 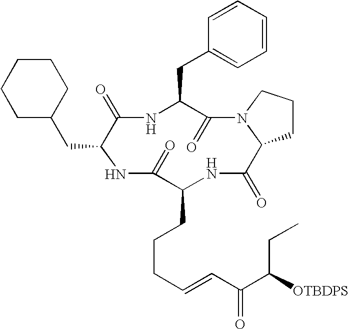 |
| Compound E127 | Compound E128 |
| 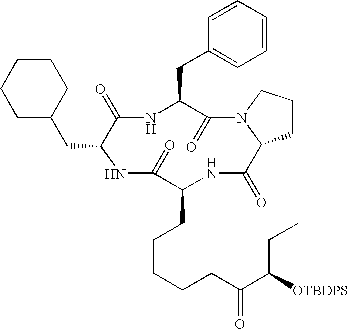 | 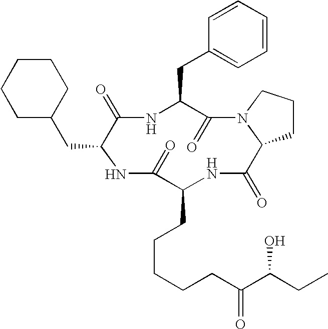 |

TABLE 2-86

| | |
|---|---|
| Compound E129 | Compound E130 |
| 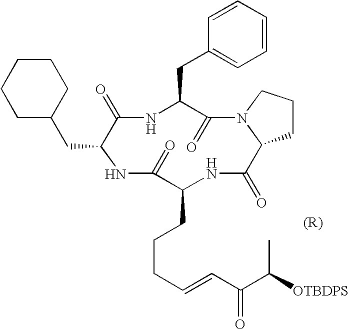 | 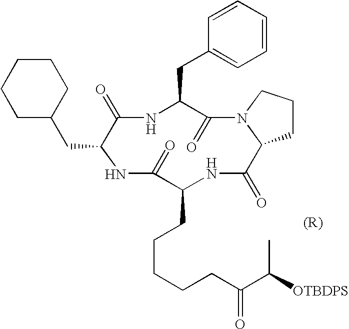 |
| Compound E131 | Compound E132 |
| 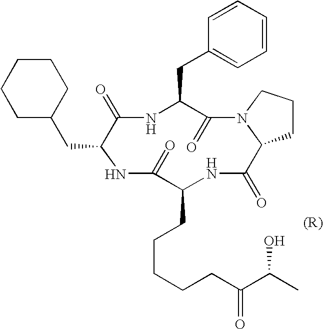 | 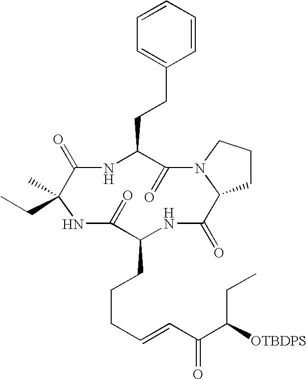 |
| Compound E133 | Compound E134 |
| 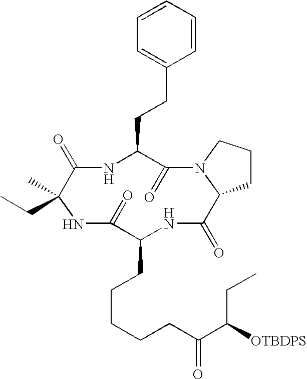 | 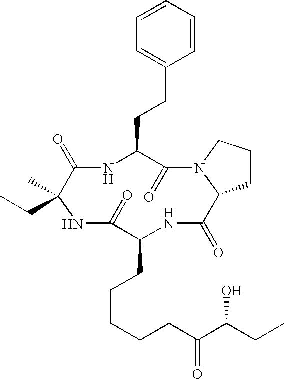 |
| Compound E135 | Compound E136 |
| 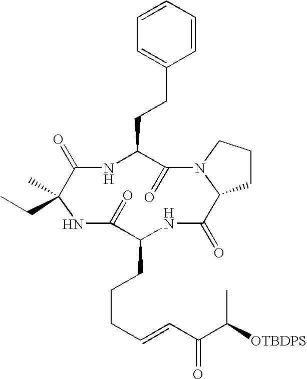 | 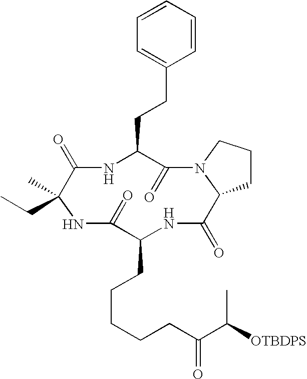 |

TABLE 2-87

| | |
|---|---|
| Compound E137 | Compound E138 |
| 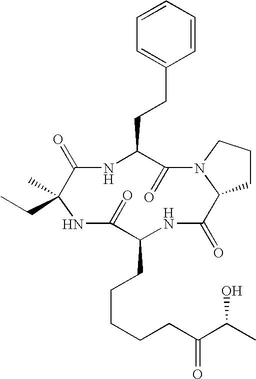 | 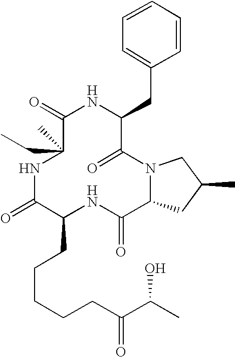 |
| Compound E139 | Compound E140 |
| 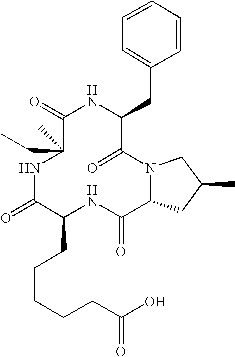 | 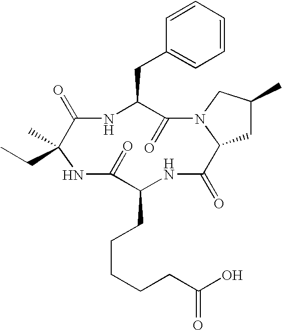 |
| Compound E141 | Compound E142 |
| 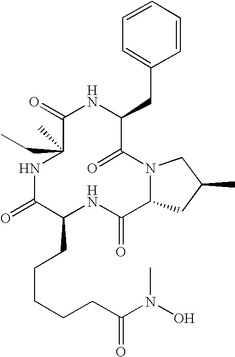 | 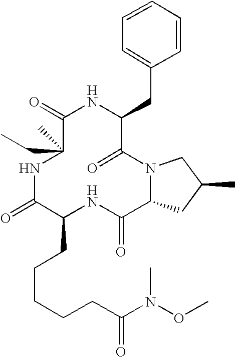 |
| Compound E143 | Compound E144 |
| 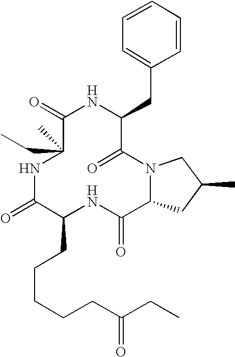 | 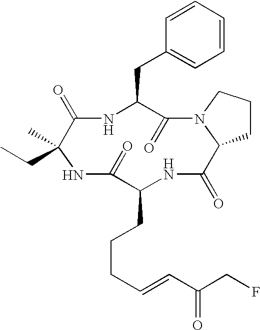 |

TABLE 2-88

| | |
|---|---|
| Compound E145 | Compound E146 |
| 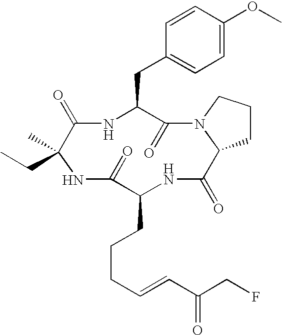 | 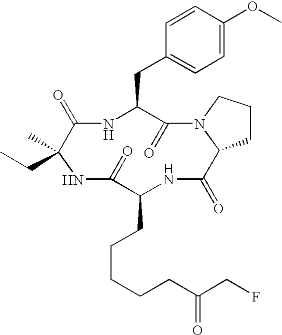 |
| Compound E147 | Compound E148 |
| 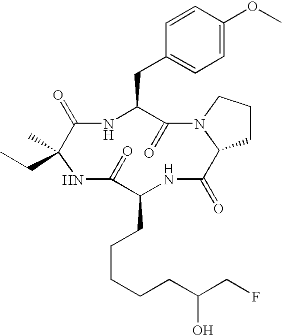 | 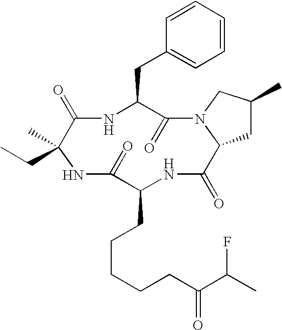 |
| Compound E149 | |
| 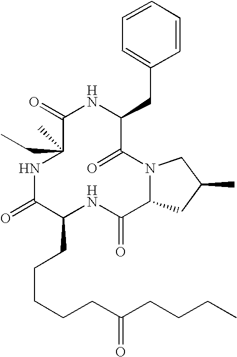 | |

TABLE 2-89

| | |
|---|---|
| Compound E150 | Compound E151 |
| 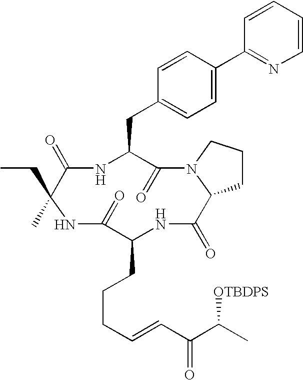 | 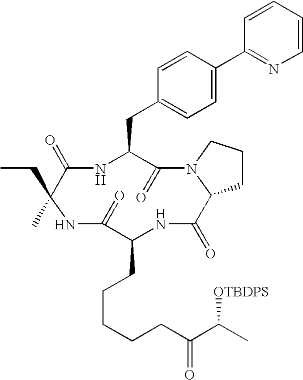 |
| Compound E152 | Compound E153 |
| 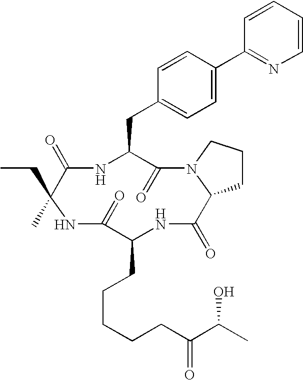 | 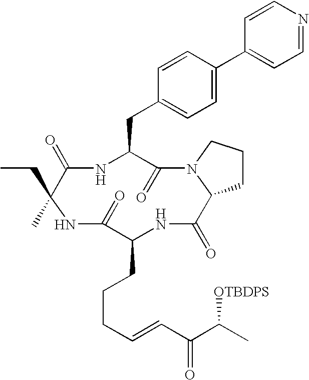 |
| Compound E154 | Compound E155 |
| 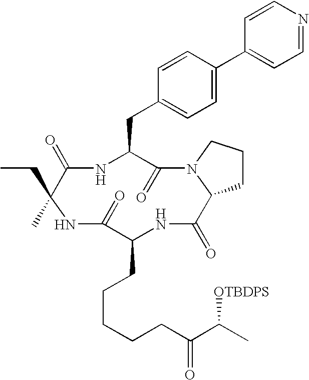 | 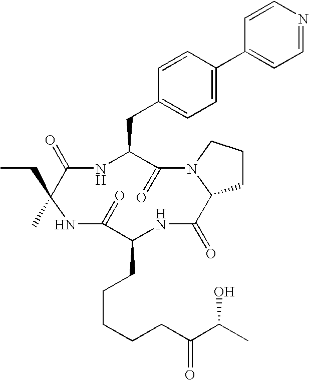 |
| Compound E156 | Compound E157 |
| 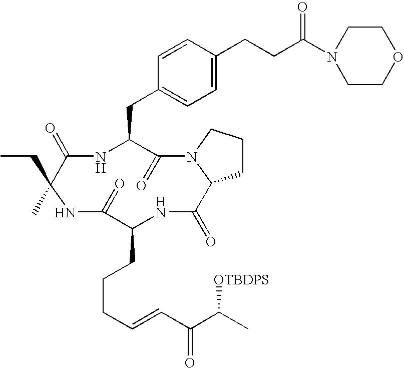 | 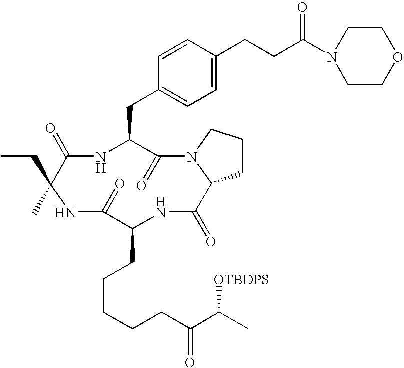 |

TABLE 2-90

| | |
|---|---|
| Compound E158 | Compound E159 |
| 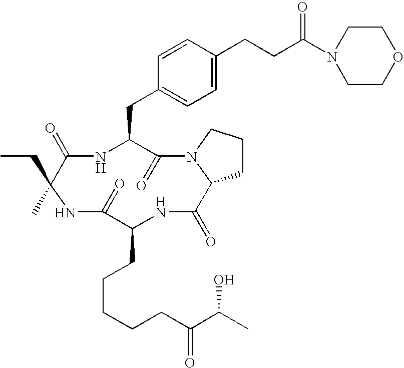 | 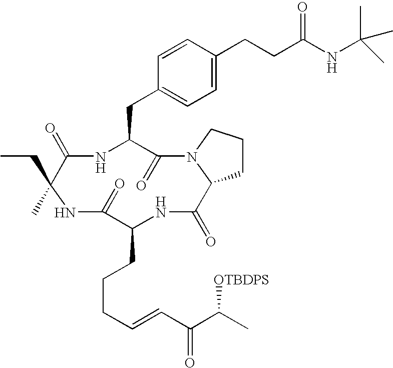 |
| Compound E160 | Compound E161 |
| 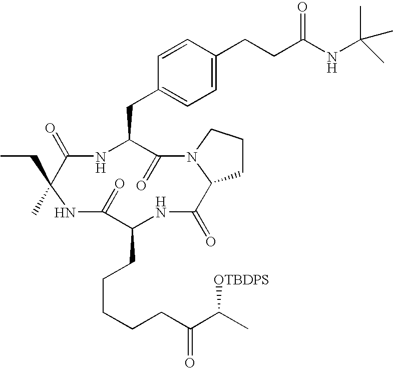 | 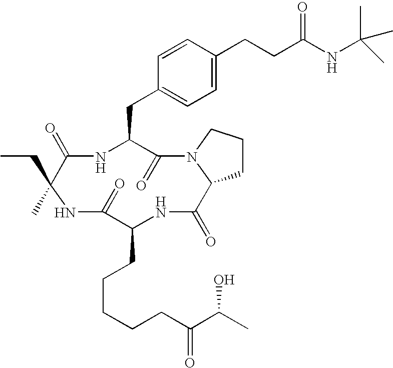 |
| Compound E162 | Compound E163 |
| 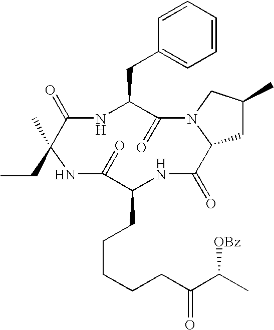 | 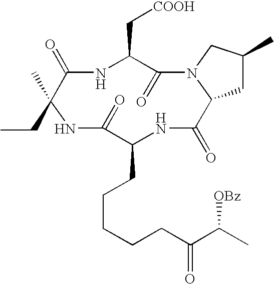 |
| Compound E164 | Compound E165 |
| 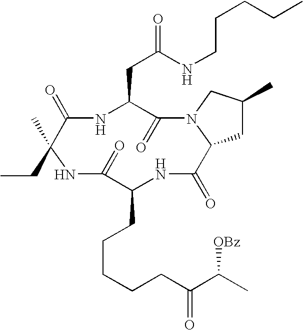 | 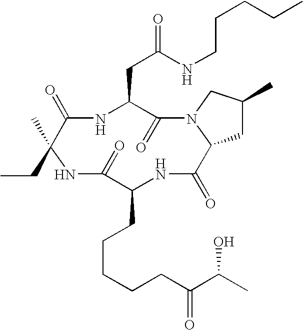 |

TABLE 2-91

| | |
|---|---|
| Compound E166 | Compound E167 |
| 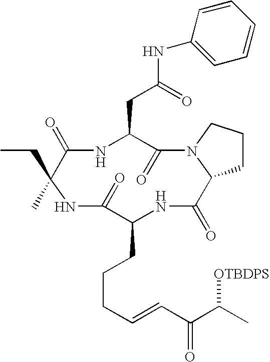 | 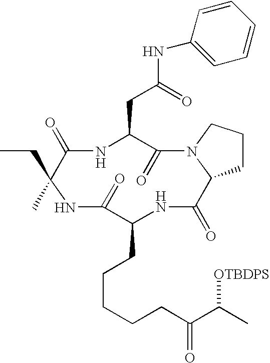 |
| Compound E168 | Compound E169 |
| 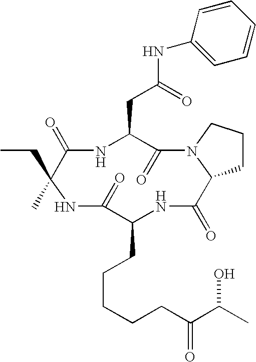 | 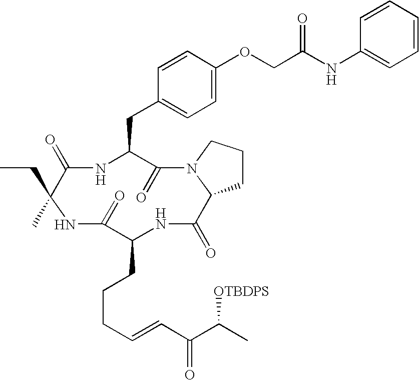 |
| Compound E170 | Compound E171 |
| 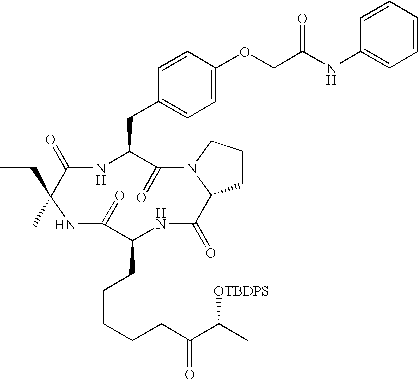 | 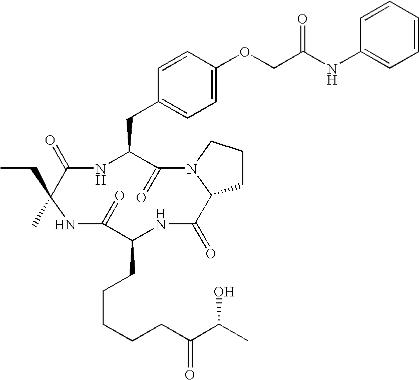 |
| Compound E172 | Compound E173 |
| 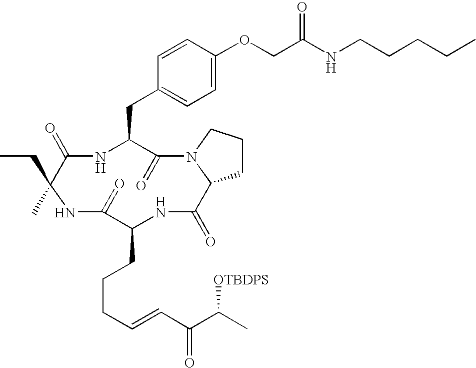 | 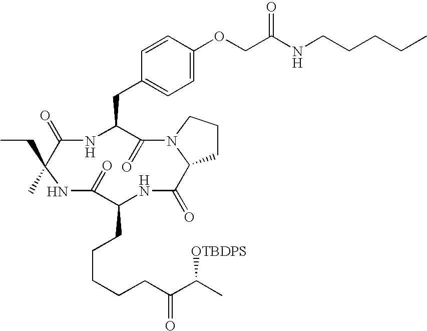 |

TABLE 2-92

| | |
|---|---|
| Compound E174 | Compound E175 |
| 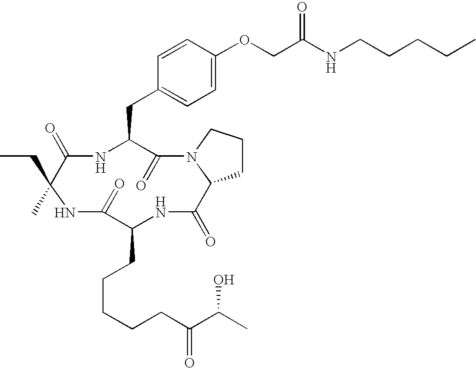 | 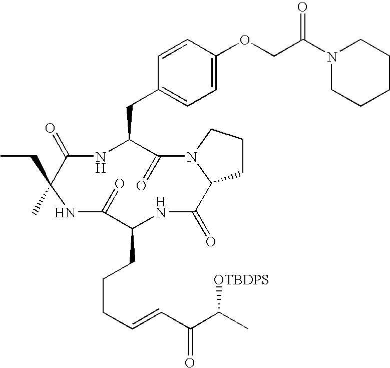 |
| Compound E176 | Compound E177 |
| 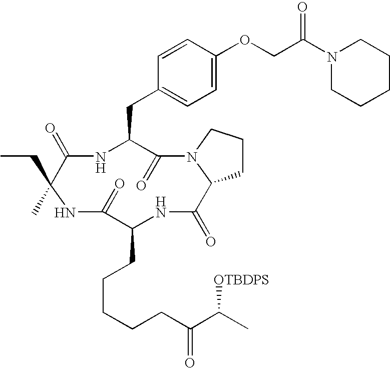 | 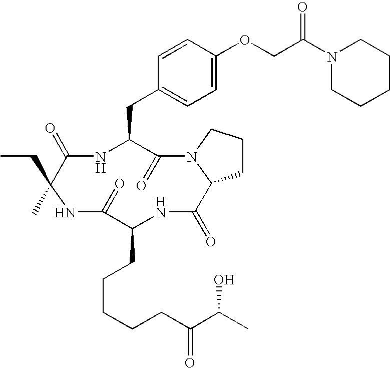 |
| Compound E178 | Compound E179 |
| 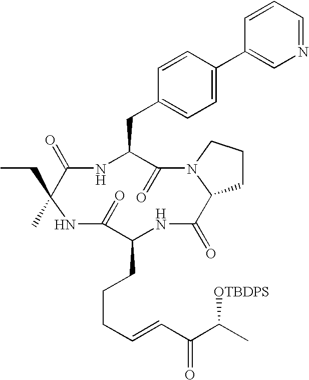 | 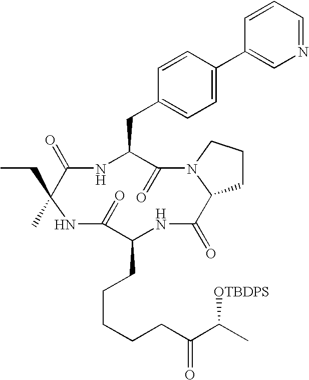 |
| Compound E180 | Compound E181 |
| 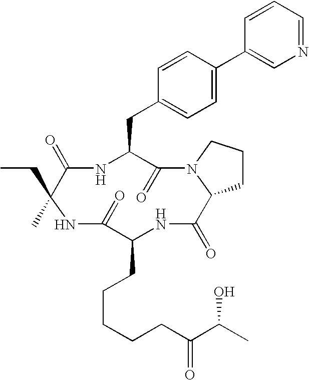 | 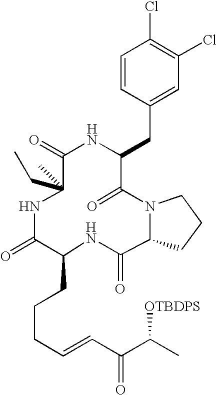 |

TABLE 2-93

| | |
|---|---|
| Compound E182 | Compound E183 |
| 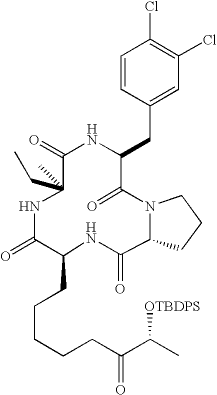 | 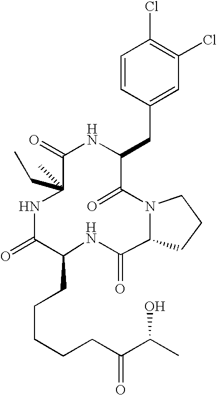 |
| Compound E184 | Compound E185 |
| 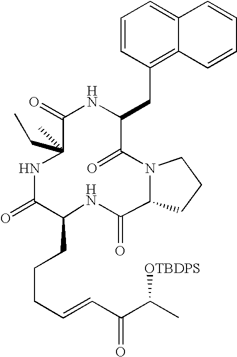 | 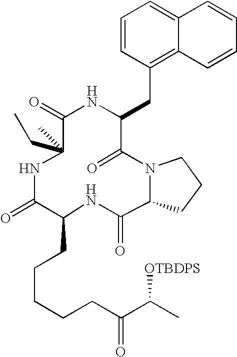 |
| Compound E186 | Compound E187 |
| 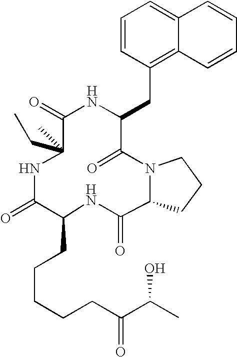 | 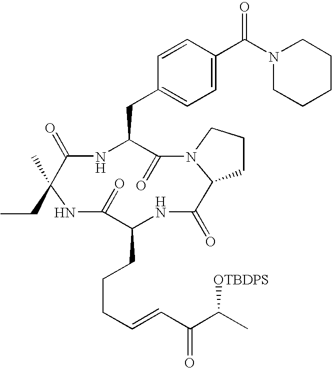 |
| Compound E188 | Compound E189 |
| 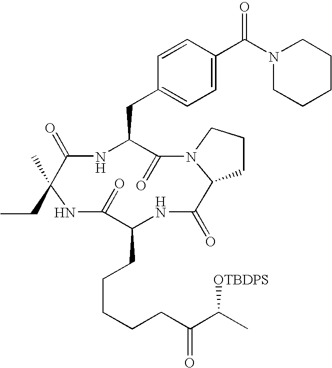 | 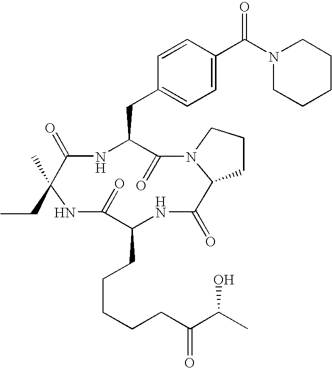 |

TABLE 2-94

| | |
|---|---|
| Compound E190 | Compound E191 |
| 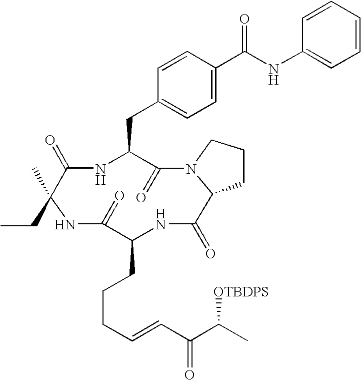 | 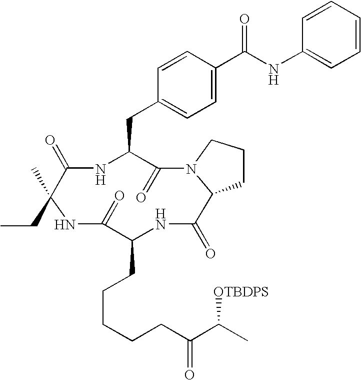 |
| Compound E192 | Compound E193 |
| 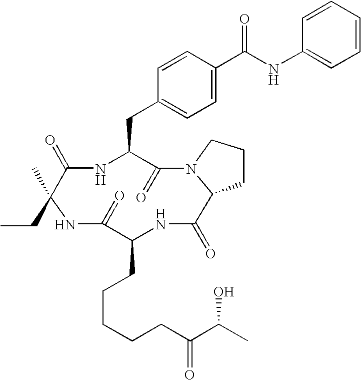 | 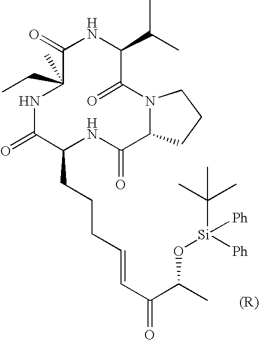 |
| Compound E194 | Compound E195 |
| 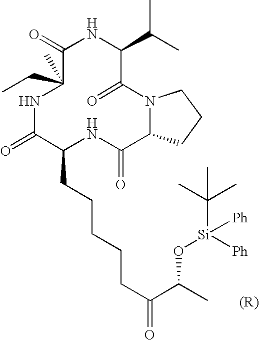 | 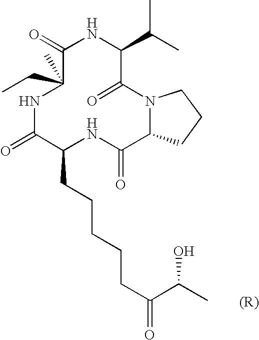 |
| Compound E196 | Compound E197 |
| 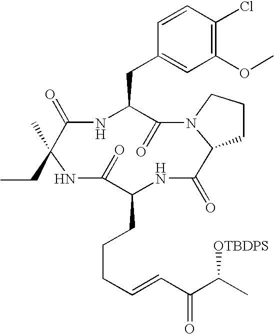 | 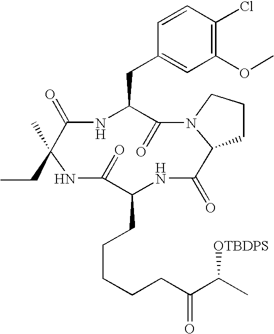 |

TABLE 2-95

| | |
|---|---|
| Compound E198 | Compound E199 |
| 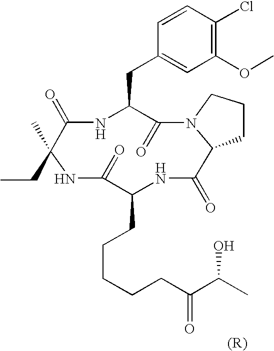 | 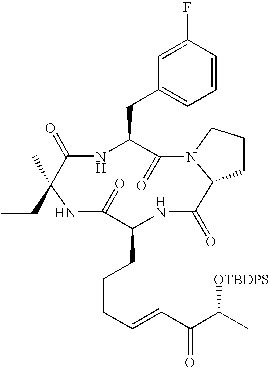 |
| Compound E200 | Compound E201 |
| 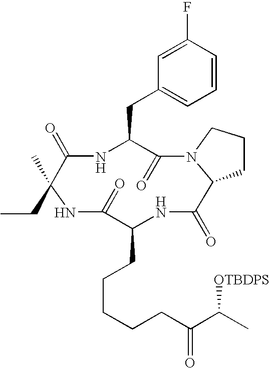 | 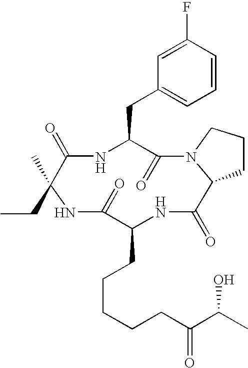 |
| Compound E202 | Compound E203 |
| 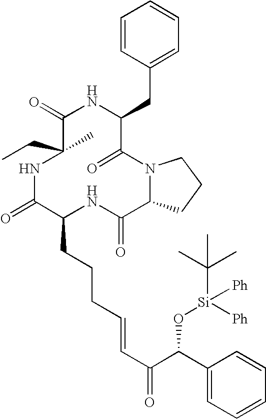 | 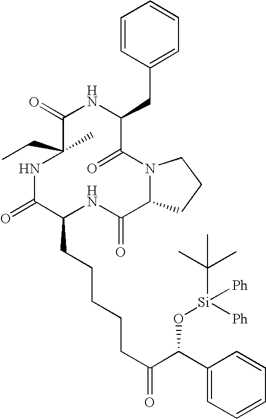 |
| Compound E204 | Compound E205 |
| 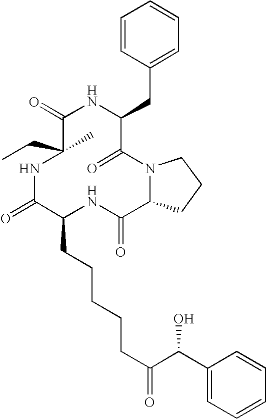 | 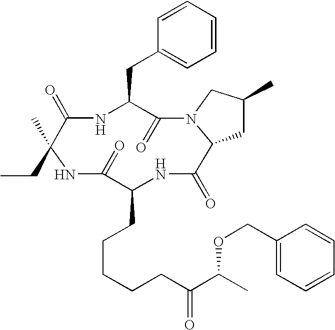 |

TABLE 2-96

| | |
|---|---|
| Compound E206 | Compound E207 |
| 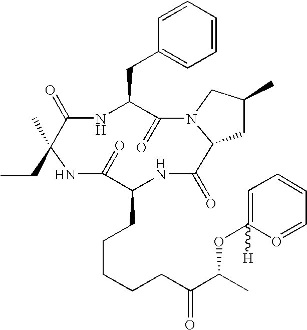 | 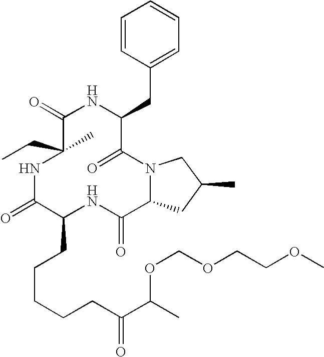 |
| Compound E208 | Compound E209 |
| 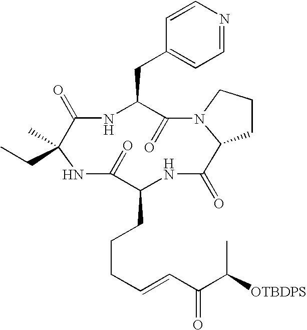 | 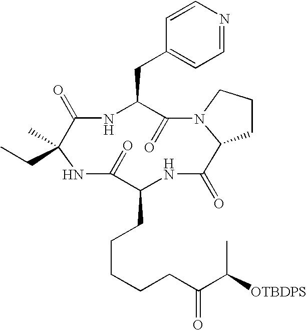 |
| Compound E210 | Compound E211 |
| 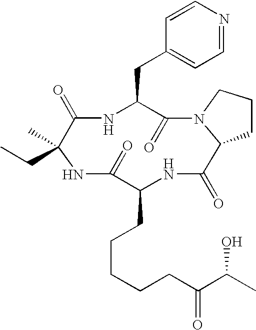 | 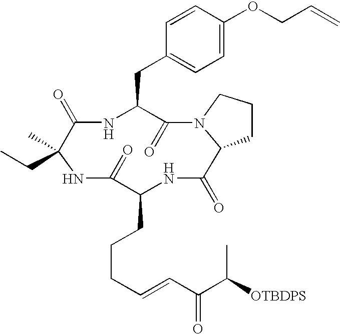 |
| Compound E212 | Compound E213 |
| 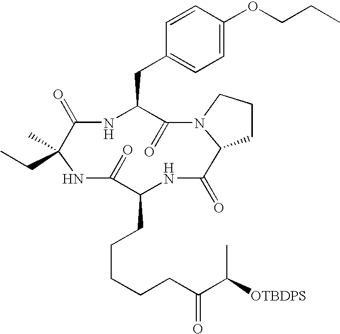 | 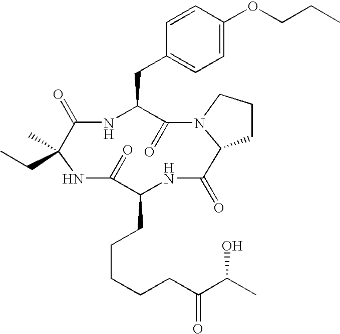 |

TABLE 2-97

| | |
|---|---|
| Compound E214 | Compound E215 |
| 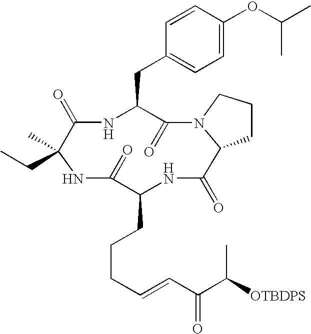 | 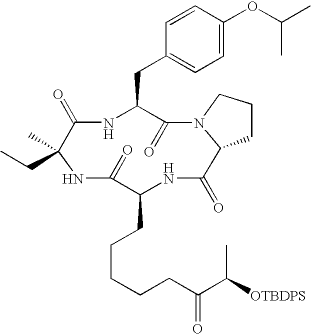 |
| Compound E216 | Compound E217 |
| 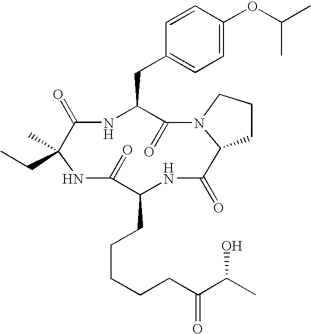 | 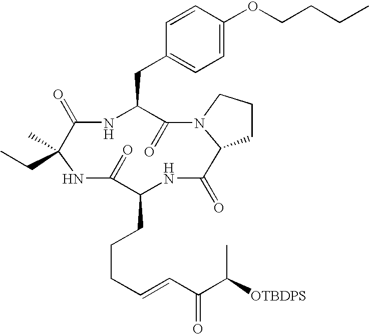 |
| Compound E218 | Compound E219 |
| 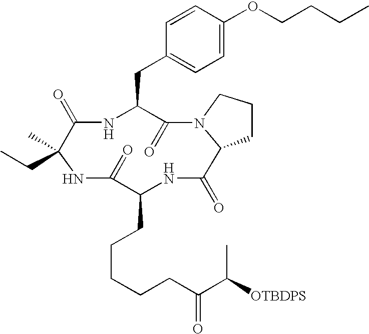 | 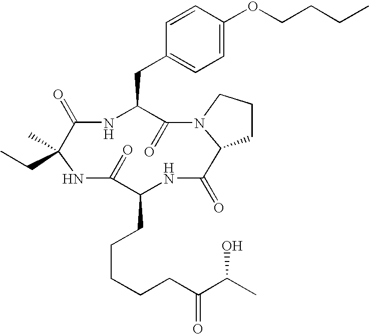 |
| Compound E220 | Compound E221 |
| 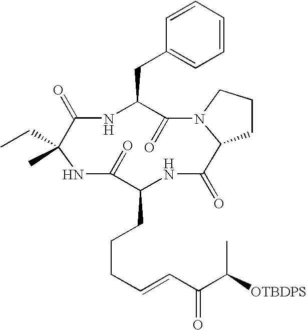 | 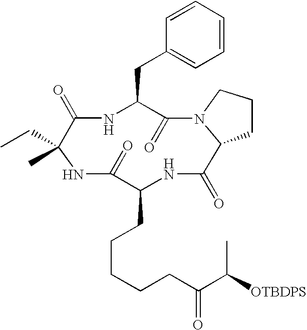 |

TABLE 2-98

| | |
|---|---|
| Compound E222 | Compound E223 |
| 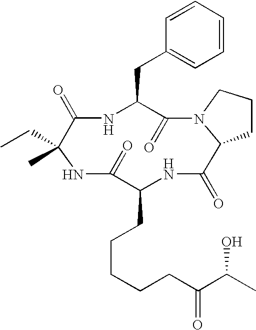 | 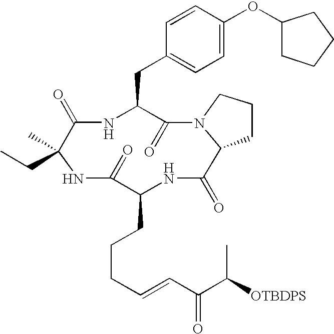 |
| Compound E224 | Compound E225 |
| 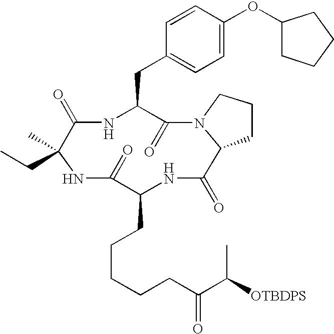 | 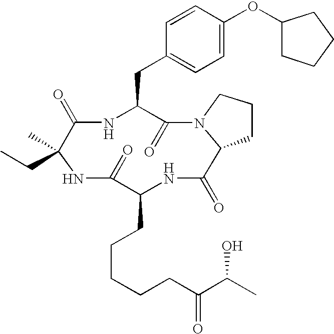 |
| Compound E226 | Compound E227 |
| 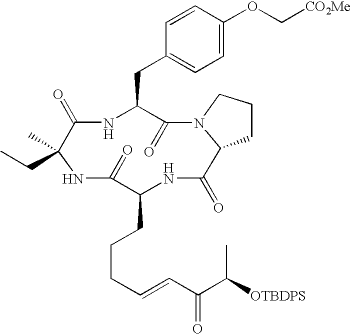 | 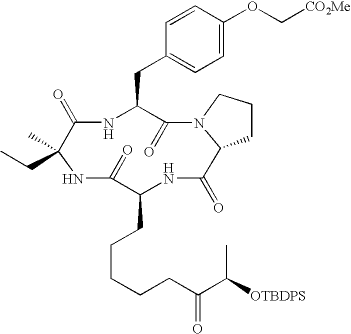 |
| Compound E228 | Compound E229 |
| 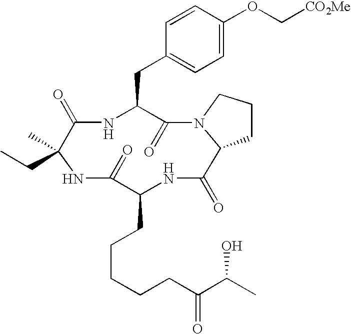 | 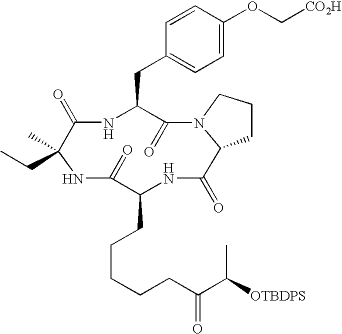 |

TABLE 2-99

| | |
|---|---|
| Compound E230 | Compound E231 |
| 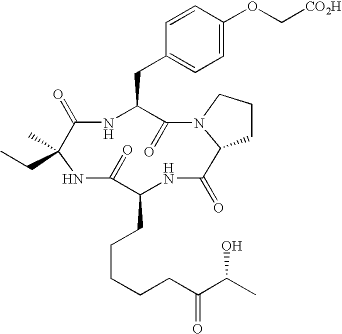 | 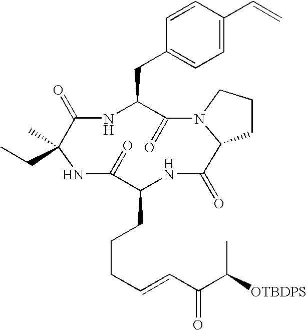 |
| Compound E232 | Compound E233 |
| 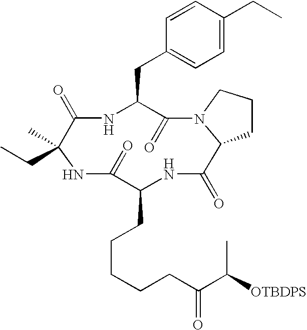 | 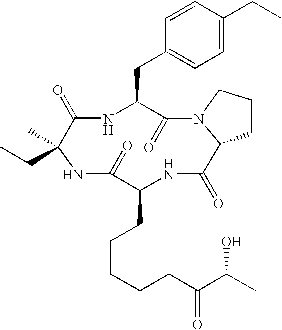 |
| Compound E234 | Compound E235 |
| 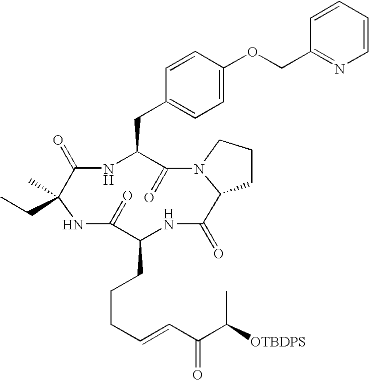 | 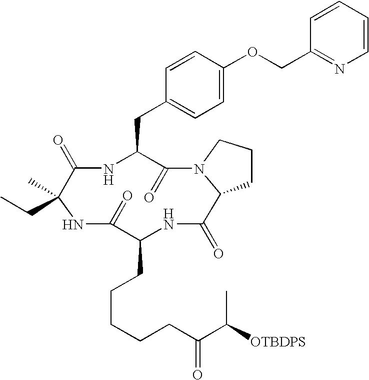 |
| Compound E236 | Compound E237 |
| 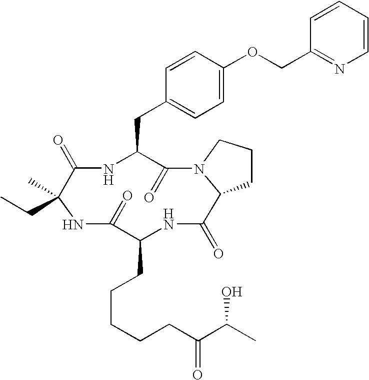 | 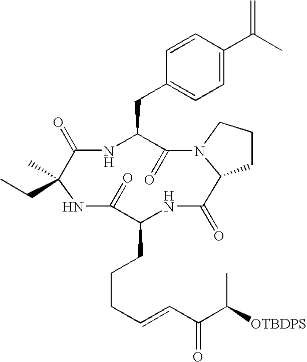 |

TABLE 2-100

| | |
|---|---|
| Compound E238 | Compound E239 |
| 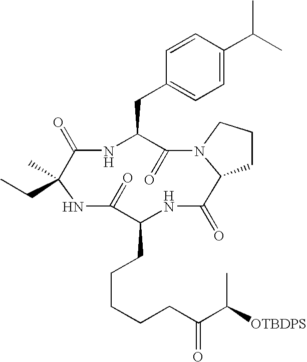 | 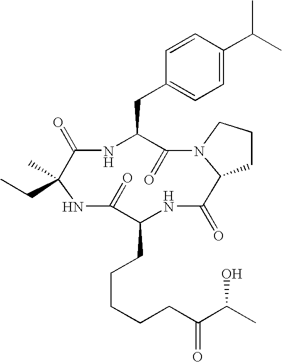 |
| Compound E240 | Compound E241 |
| 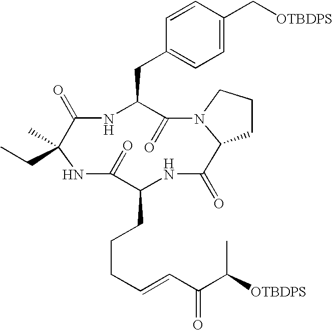 | 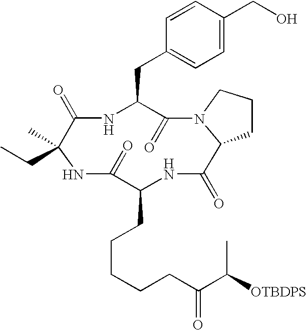 |
| Compound E242 | Compound E243 |
| 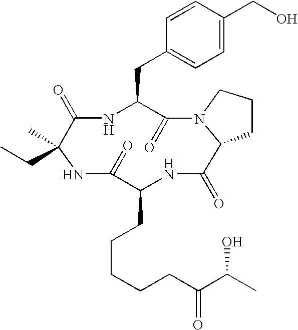 | 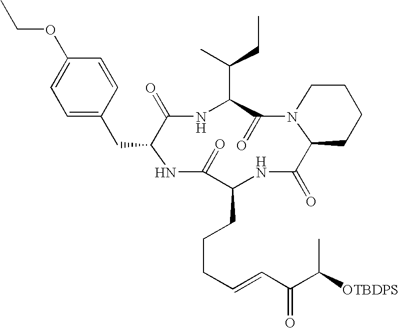 |
| Compound E244 | Compound E245 |
| 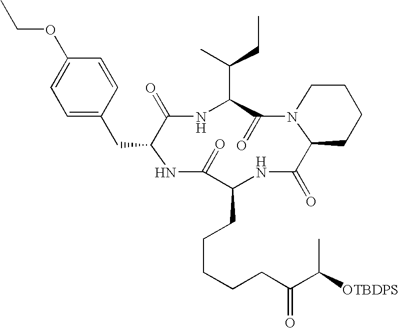 | 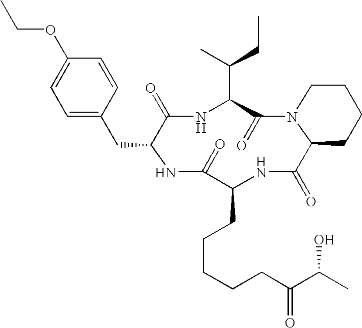 |

TABLE 2-101

| | |
|---|---|
| Compound E246 | Compound E247 |
| 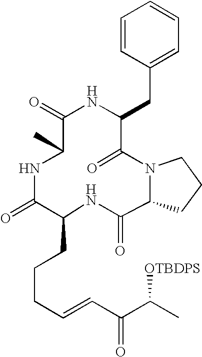 | 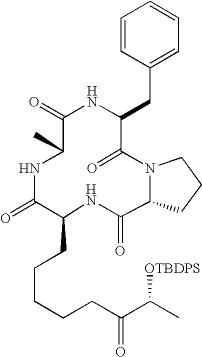 |
| Compound E248 | Compound E249 |
| 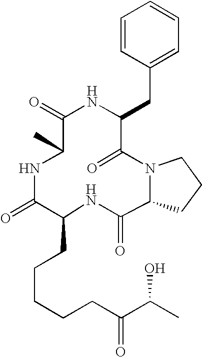 | 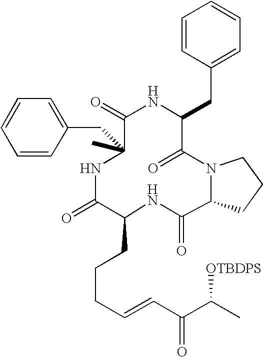 |
| Compound E250 | Compound E251 |
| 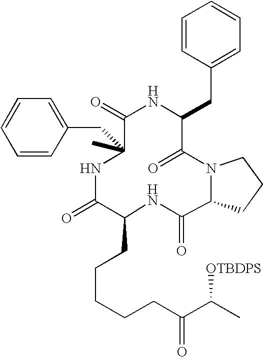 | 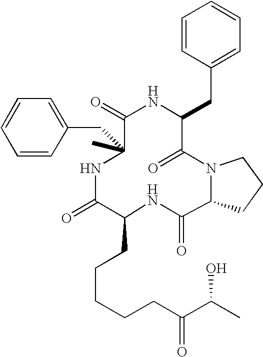 |
| Compound E252 | Compound E253 |
| 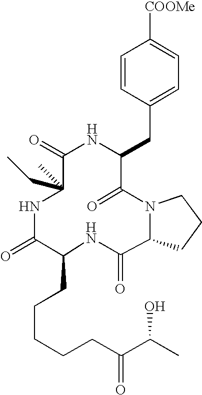 | 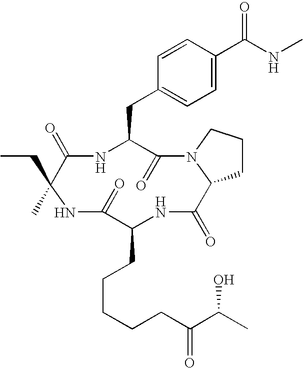 |

TABLE 2-102

| | |
|---|---|
| Compound E254 | Compound E255 |
| 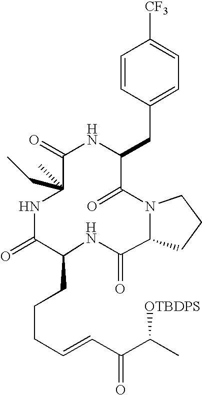 | 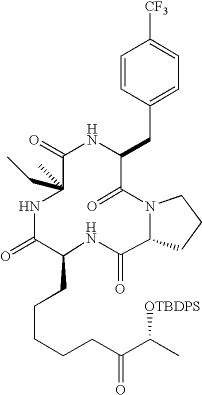 |
| Compound E256 | Compound E257 |
| 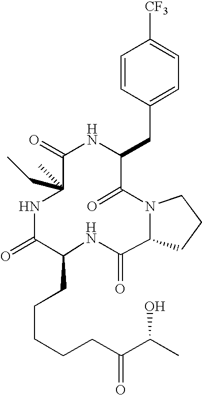 | 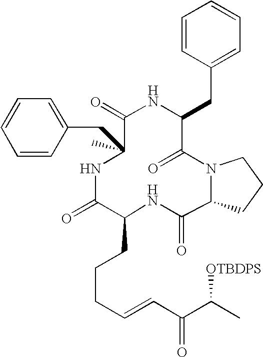 |
| Compound E258 | Compound E259 |
| 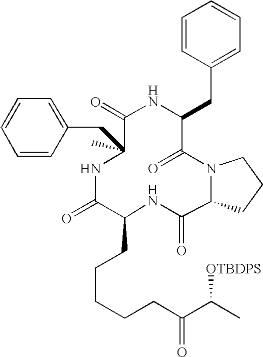 | 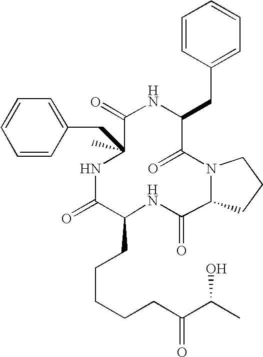 |
| Compound E260 | Compound E261 |
| 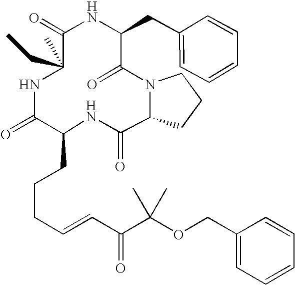 | 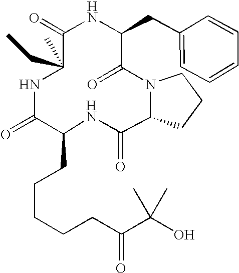 |

TABLE 2-103

| | |
|---|---|
| Compound E262 | Compound E263 |
| 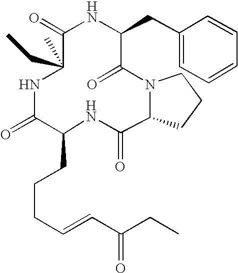 | 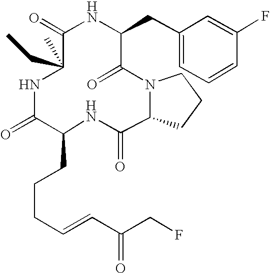 |
| Compound E264 | Compound E265 |
| 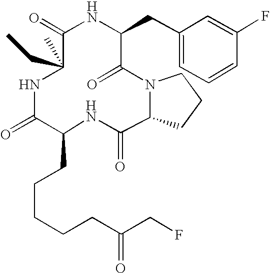 | 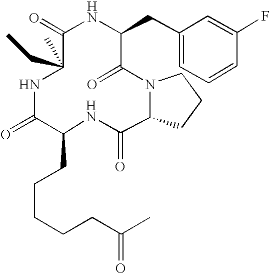 |
| Compound E266 | Compound E267 |
| 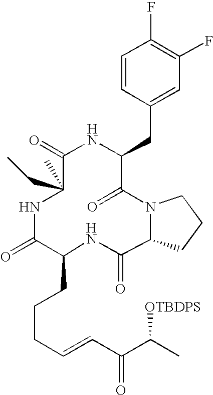 | 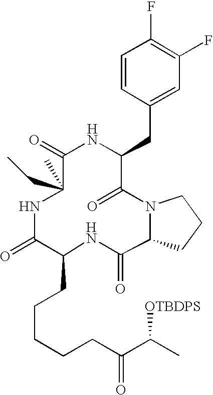 |
| Compound E268 | Compound E269 |
| 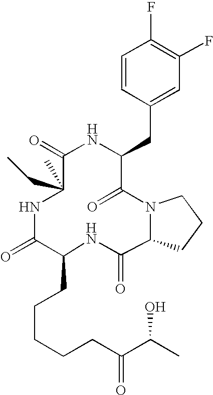 | 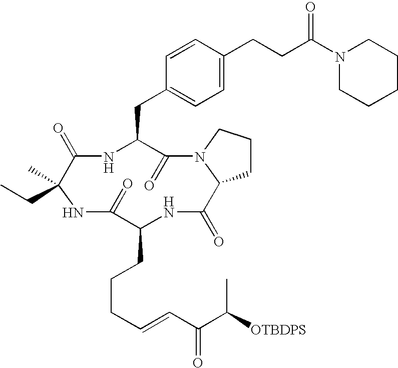 |

TABLE 2-104

| | |
|---|---|
| Compound E270 | Compound E271 |
| 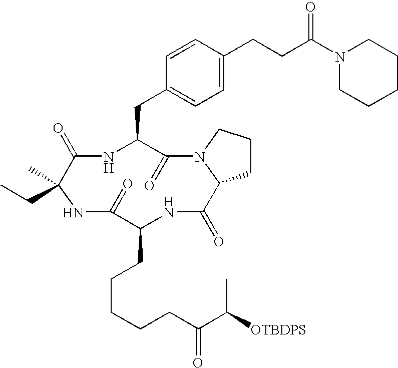 | 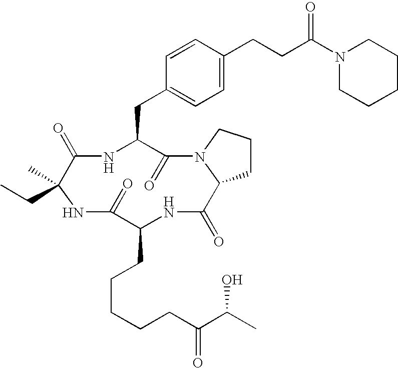 |
| Compound E272 | Compound E273 |
| 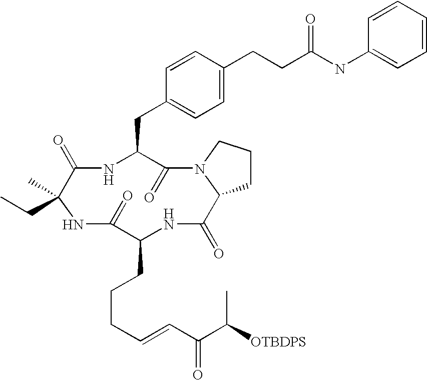 | 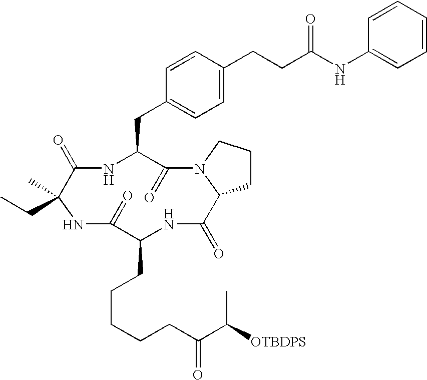 |
| Compound E274 | Compound E275 |
| 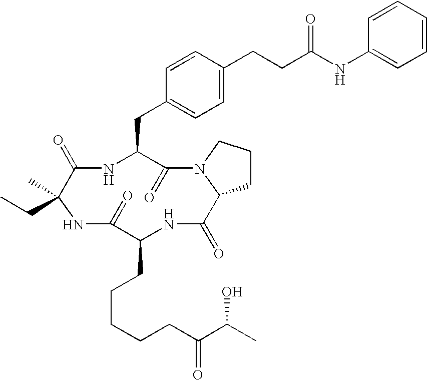 | 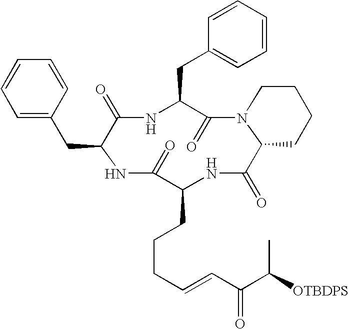 |
| Compound E276 | Compound E277 |
| 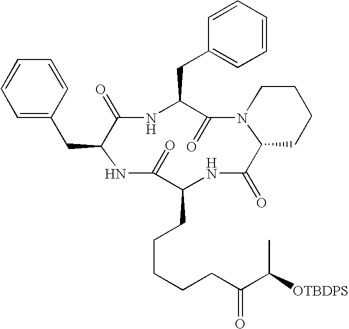 | 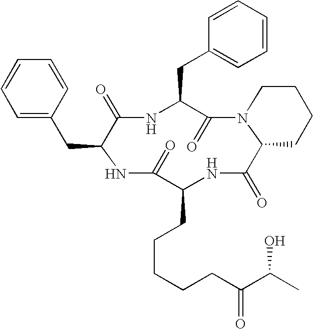 |

TABLE 2-105

| | |
|---|---|
| Compound E278 | Compound E279 |
| 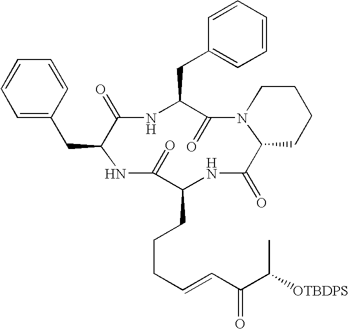 | 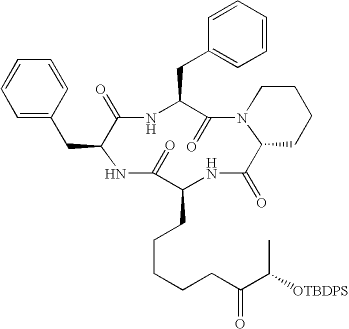 |
| Compound E280 | Compound E281 |
| 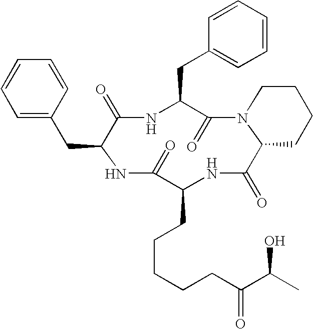 | 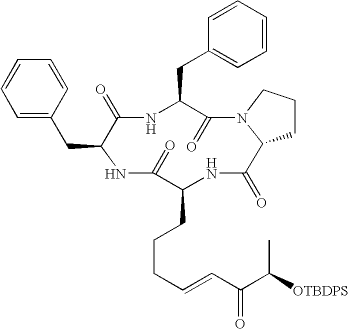 |
| Compound E282 | Compound E283 |
| 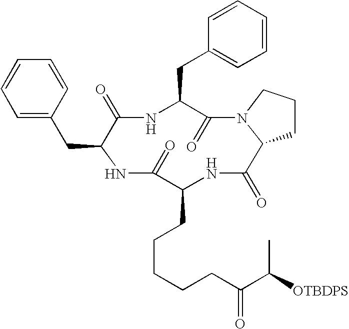 | 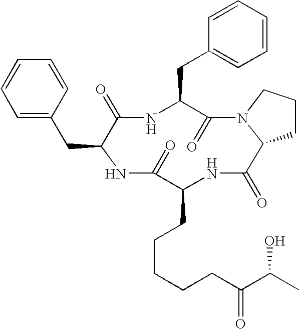 |
| Compound E284 | Compound E285 |
| 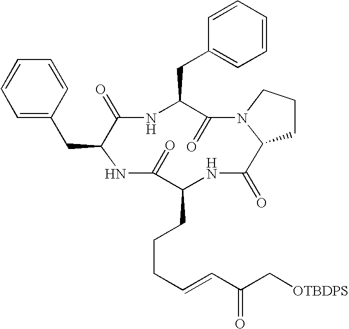 | 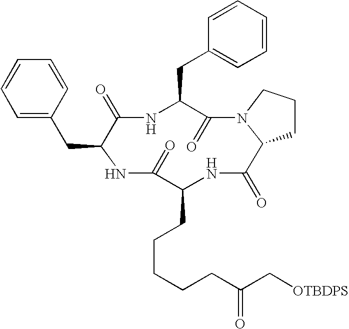 |

TABLE 2-106

| | |
|---|---|
| Compound E286 | Compound E287 |
| 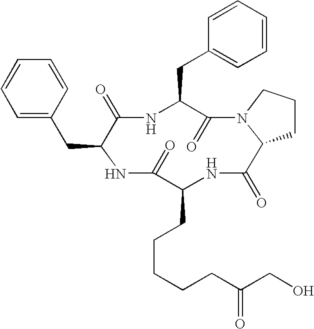 | 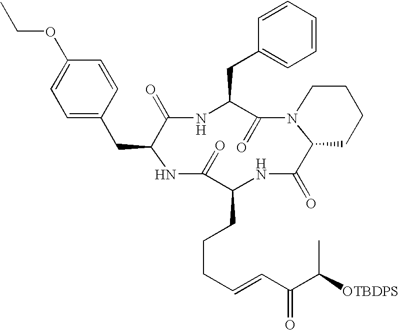 |
| Compound E288 | Compound E289 |
| 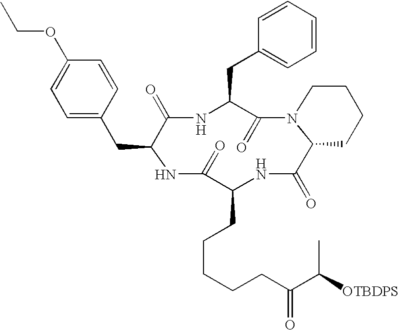 | 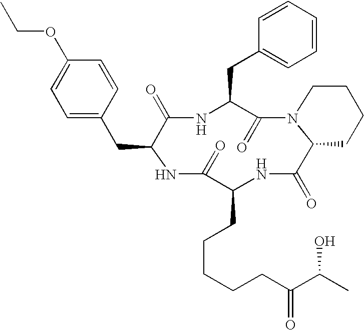 |
| Compound E290 | Compound E291 |
| 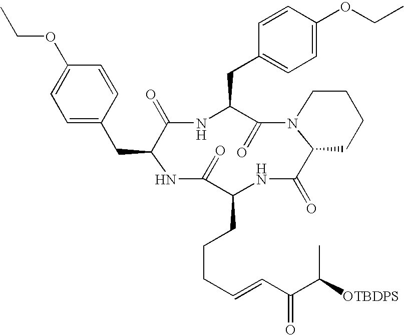 | 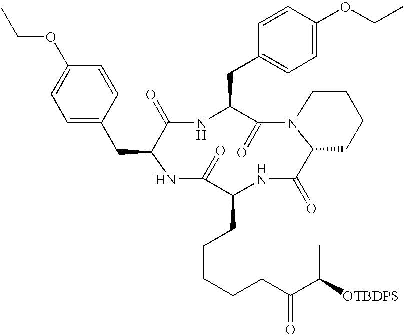 |
| Compound E292 | Compound E293 |
| 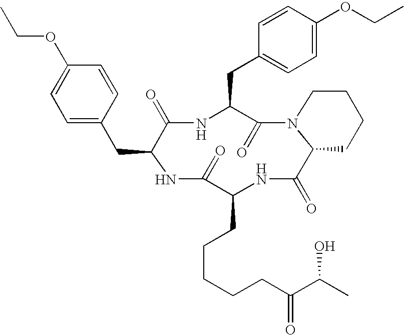 | 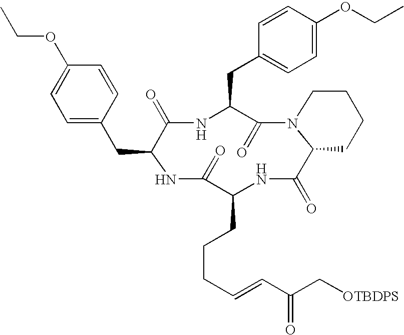 |

TABLE 2-107

| | |
|---|---|
| Compound E294 | Compound E295 |
| 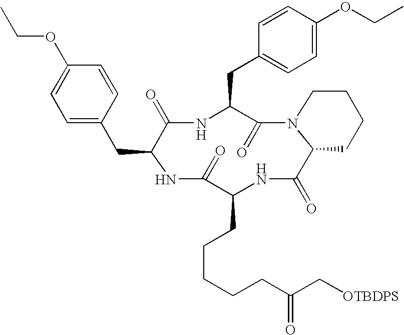 | 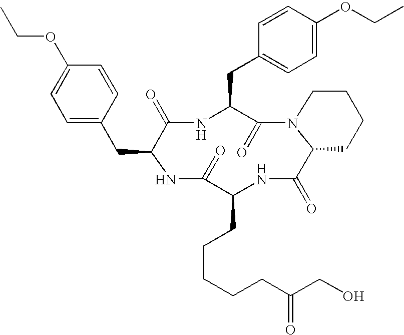 |
| Compound E296 | Compound E297 |
| 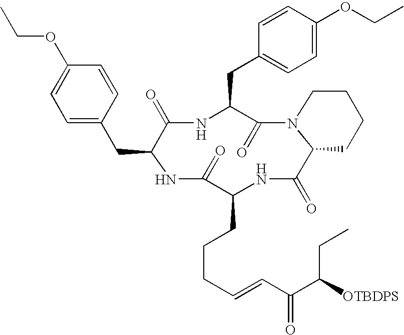 | 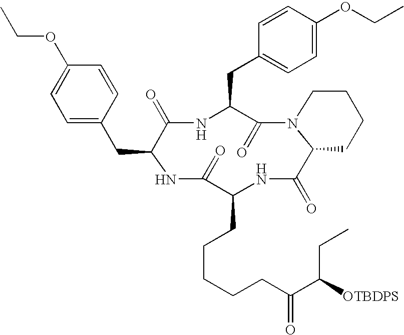 |
| Compound E298 | Compound E299 |
| 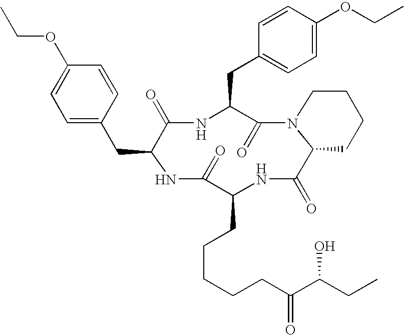 | 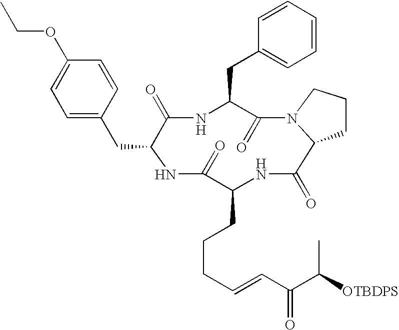 |
| Compound E300 | Compound E301 |
| 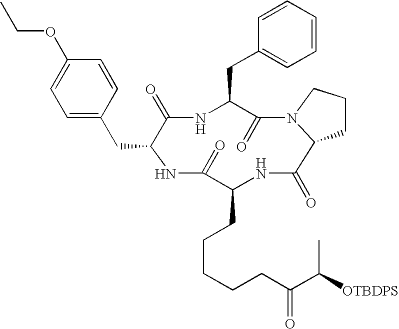 | 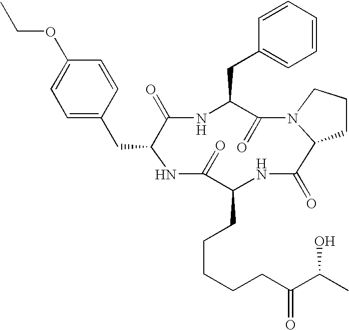 |

TABLE 2-108

| | |
|---|---|
| Compound E302 | Compound E303 |
| 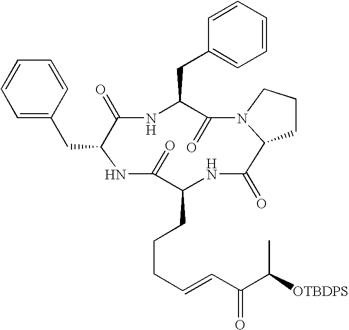 | 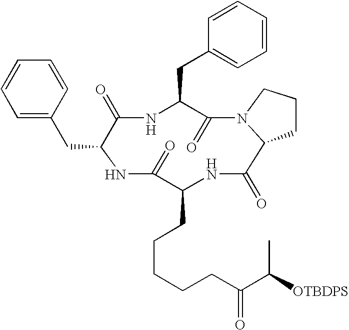 |
| Compound E304 | Compound E305 |
| 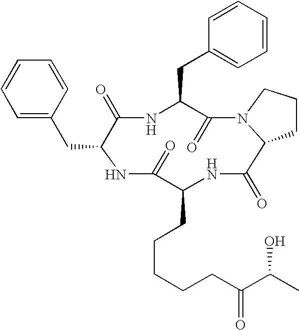 | 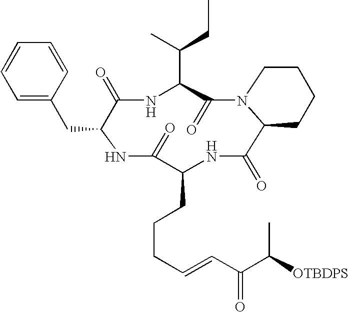 |
| Compound E306 | Compound E307 |
| 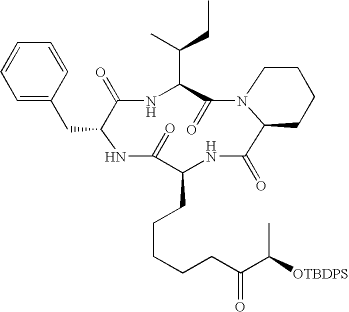 | 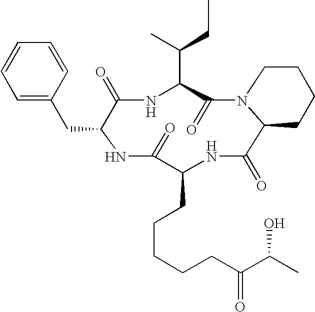 |
| Compound E308 | Compound E309 |
| 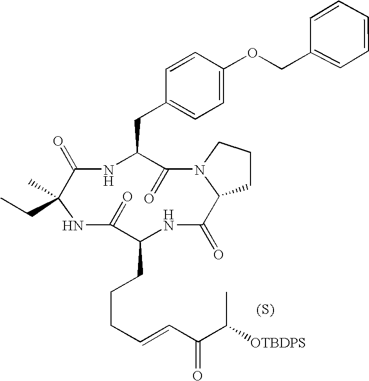 | 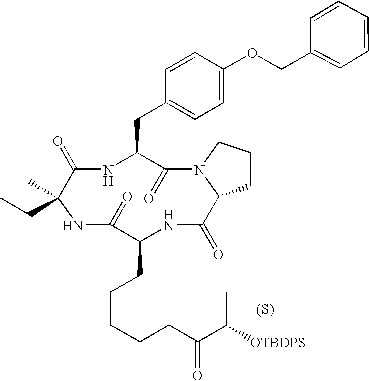 |

TABLE 2-109

| | |
|---|---|
| Compound E310 | Compound E311 |
| 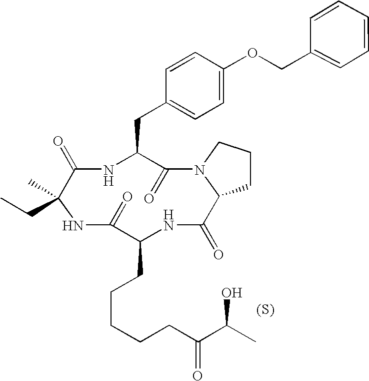 | 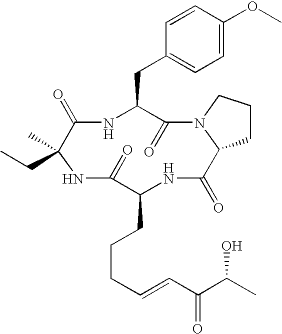 |
| Compound E312 | Compound E313 |
| 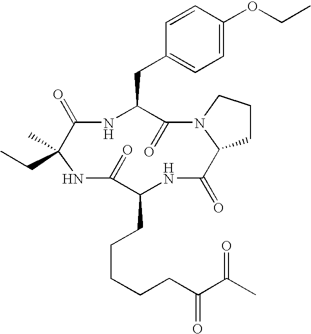 | 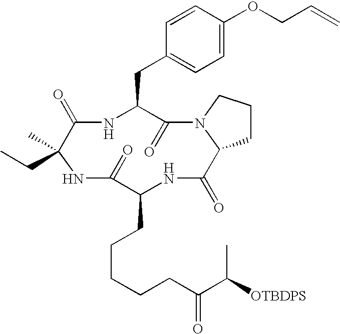 |
| Compound E314 | |
| 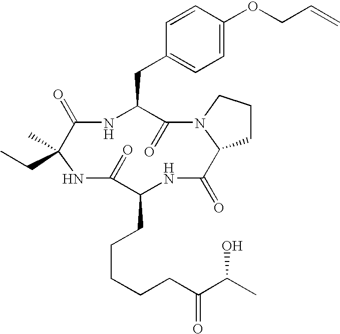 | |

1. A cyclic tetrapeptide compound of the formula (I): 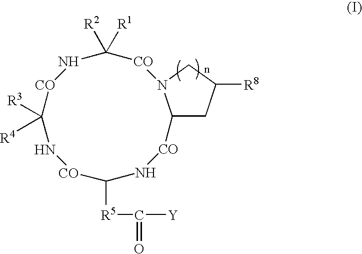
wherein
R¹ is hydrogen,
R² is lower alkyl, aryl, ar(lower)alkyl optionally substituted with one or more suitable substituent(s), heterocyclic(lower)alkyl, cyclo(lower)alkyl(lower)alkyl, lower alkylcarbamoyl(lower)alkyl, or arylcarbamoyl(lower)alkyl,
R³ and R⁴ are each independently hydrogen, lower alkyl, ar(lower)alkyl optionally substituted with one or more suitable substituent(s), heterocyclic(lower)alkyl optionally substituted with one or more suitable substituent(s) or cyclo(lower)alkyl(lower)alkyl, or
R³ and R⁴ are linked together to form lower alkylene or condensed ring, or one of R³ and R⁴ is linked to the adjacent nitrogen atom to form a ring, R⁵ is lower alkylene or lower alkenylene,
Y is 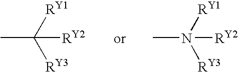
[wherein R^{Y1} is hydrogen, halogen or optionally protected hydroxy,
R^{Y2} is hydrogen, halogen, lower alkyl or phenyl, and
R^{Y3} is hydrogen or lower alkyl],
R⁸ is hydrogen or lower alkyl, and
n is an integer of 1 or 2,
providing that,
when R³ is methyl, R⁴ is methyl or ethyl, R⁵ is pentylene, R⁸ is hydrogen, n is 1, R^{Y1} is optionally substituted hydroxy, R^{Y2} is methyl and R^{Y3} is hydrogen, then R² is not unsubstituted benzyl,
or a salt thereof.

2. The cyclic tetrapeptide compound of claim 1, wherein
R² is phenylcarbamoyl(lower)alkyl; lower alkylcarbamoyl(lower)alkyl; or phenyl(lower)alkyl optionally substituted with one or more suitable substituent(s) selected from the group consisting of lower alkyl, halo (lower)alkyl, lower alkoxy, ar(lower)alkoxy, cyano, hydroxy, halogen, amino, lower alkanoylamino, lower alkylsulfonylamino, aryl, cyclo(lower)alkyloxy, carboxy(lower)alkoxy, heterocyclic(lower)alkoxy; lower alkenyloxy, hydroxy(lower)alkyl, arylcarbamoyl, heterocycliccarbonyl, lower(alkyl)carbamoyl(lower)alkoxy, arylcarbamoyl(lower)alkoxy, lower (alkyl) carbamoyl(lower)alkyl, heterocyclic group, lower alkoxycarbonyl, lower alkoxycarbonyl(lower)alkoxy, lower alkylcarbamoyl, heterocycliccarbonyl(lower)alkyl, heterocycliccarbonyl(lower)alkoxy, aryl(lower)alkoxy and phenylcarbamoyl(lower)alkyl,
R³ is hydrogen or lower alkyl,
R⁴ is lower alkyl or phenyl(lower)alkyl substituted with lower alkoxy,
R⁵ is lower alkylene, 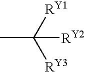
[wherein R^{Y1} is hydrogen or hydroxy, R^{Y2} is halogen or lower alkyl and R^{Y3} is hydrogen] and
R⁸ is hydrogen or lower alkyl.

3. The cyclic tetrapeptide compound of claim 2, wherein
R² is phenyl (lower) alkyl substituted with a substituent selected from the group consisting of lower alkyl, halo(lower)alkyl, lower alkoxy, phenyl (lower) alkyloxy, cyano, hydroxy, halogen, amino, lower alkanoylamino, (lower)alkylsulfonylamino, phenyl, cyclo(lower)alkyloxy, carboxy(lower)alkyloxy, pyridyl(lower)alkyloxy, lower alkenyloxy, hydroxy(lower)alkyl, phenylcarbamoyl, piperidinocarbonyl, lower (alkyl) carbamoyl(lower)alkoxy, phenylcarbamoyl(lower)alkoxy, lower (alkyl)carbamoyl(lower)alkyl, pyridyl, lower alkoxycarbonyl, lower alkoxycarbonyl(lower)alkoxy, lower alkylcarbamoyl, morpholinocarbonyl(lower)alkyl, piperidinocarbonyl(lower)alkoxy, phenyl (lower) alkoxy and phenylcarbamoyl(lower)alkyl,
R³ is lower alkyl,
R⁴ is lower alkyl, and
R⁵ is lower alkylene.

4. A pharmaceutical composition containing the cyclic tetrapeptide compound of any of claims 1 to 3 as an active ingredient, in association with a pharmaceutically acceptable, substantially non-toxic carrier or excipient.

5. The cyclic tetrapeptide compound of any of claims 1 to 3 for use as a medicament.

6. A histone deacetylase inhibitor comprising a cyclic tetrapeptide compound of the formula (I): 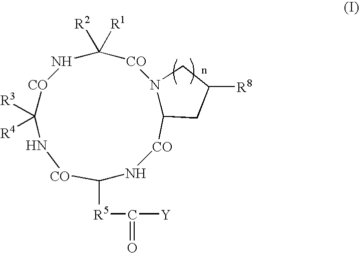
wherein
R¹ is hydrogen,
R² is lower alkyl, aryl, ar(lower)alkyl optionally substituted with one or more suitable substituent(s), heterocyclic(lower)alkyl, cyclo(lower)alkyl (lower)alkyl, lower alkylcarbamoyl(lower)alkyl or arylcarbamoyl(lower)alkyl,
R³ and R⁴ are each independently hydrogen, lower alkyl, ar (lower)alkyl a optionally substituted with one or more suitable substituent(s), heterocyclic(lower)alkyl optionally substituted with one or more suitable substituent(s) or cyclo(lower)alkyl(lower)alkyl, or
R³ and R⁴ are linked together to form lower alkylene or condensed ring, or one of R³ and R⁴ is linked to the adjacent nitrogen atom to form a ring, R⁵ is lower alkylene or lower alkenylene,
Y is. 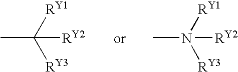
[wherein R^{Y1} is hydrogen, halogen, optionally protected hydroxy R^{Y2} is hydrogen, halogen, lower alkyl or phenyl, and
R^{Y3} is hydrogen or lower alkyl],
R⁸ is hydrogen or lower alkyl, and n is an integer of 1 or 2,
providing that,
when R³ is methyl, R⁴ is methyl or ethyl, R⁵ is pentylene, R^{Y1} is optionally substituted hydroxy, R^{Y2} is methyl and R^{Y3} is hydrogen, then R² is not unsubstituted benzyl,
or a salt thereof.

7. A method for inhibiting histone deacetylase, comprising using a cyclic tetrapeptide compound (I) of claim 6.

8. Use of a cyclic tetrapeptide compound (I) of claim 6 for the manufacture of a medicament for inhibiting histone deacetylase.

9. A pharmaceutical composition for treating or preventing inflammatory disorders, diabetes, diabetic complications, homozygous thalassemia, fibrosis, cirrhosis, acute promyelocytic leukaemia (APL), organ transplant rejections, autoimmune diseases, protozoal infections or tumors, which comprises, as an active ingredient, a cyclic tetrapeptide compound of the formula (I): 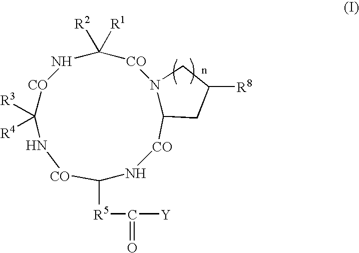
wherein
R¹ is hydrogen,
R² is lower alkyl, aryl, ar(lower)alkyl optionally substituted with one or more suitable substituent(s), heterocyclic(lower)alkyl, cyclo(lower)alkyl(lower)alkyl, lower alkylcarbamoyl(lower)alkyl or arylcarbamoyl(lower)alkyl,
R³ and R⁴ are each independently hydrogen, lower alkyl, ar(lower)alkyl optionally substituted with one or more suitable substituent(s), heterocyclic(lower)alkyl optionally substituted with one or more suitable substituent(s) or cyclo(lower)alkyl(lower)alkyl, or
R³ and R⁴ are linked together to form lower alkylene or condensed ring, or one of R³ and R⁴ is linked to the adjacent nitrogen atom to form a ring, R⁵ is lower alkylene or lower alkenylene, 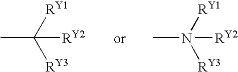
Y is
[wherein R^{Y1} is hydrogen, halogen, optionally protected hydroxy R^{Y2} is hydrogen, halogen, lower alkyl or phenyl, and
R^{Y3} is hydrogen or lower alkyl],
R⁸ is hydrogen or lower alkyl, and
n is an integer of 1 or 2, providing that,
when R³ is methyl, R⁴ is methyl or ethyl, R⁵ is pentylene, R⁸ is hydrogen, n is 1, R^{Y1} is optionally substituted hydroxy, R^{Y2} is methyl and R^{Y3} is hydrogen, then R² is not unsubstituted benzyl,
or a salt thereof.

10. A method for treating or preventing inflammatory disorders, diabetes, diabetic complications, homozygous thalassemia, fibrosis, cirrhosis, acute promyelocytic leukaemia (APL), organ transplant rejections, autoimmune diseases, protozoal infections or tumors, which comprises administering an effective amount of the cyclic tetrapeptide compound (I) of claim 1 to a human being or an animal.

11. Use of the cyclic tetrapeptide compound (I) of claim 1 for the manufacture of a medicament for treating or preventing inflammatory disorders, diabetes, diabetic complications, homozygous thalassemia, fibrosis, cirrhosis, acute promyelocytic leukaemia (APL), organ transplant rejections, autoimmune diseases, protozoal infections or tumors.

12. A commercial package comprising the pharmaceutical composition of claim 9 and a written matter associated therewith, the written matter stating that the pharmaceutical composition may or should be used for treating or preventing inflammatory disorders, diabetes, diabetic complications, homozygous thalassemia, fibrosis, cirrhosis, acute promyelocytic leukaemia (APL), organ transplant rejections, autoimmune diseases, protozoal infections or tumors.

13. A cyclic tetrapeptide compound of the formula (I′): 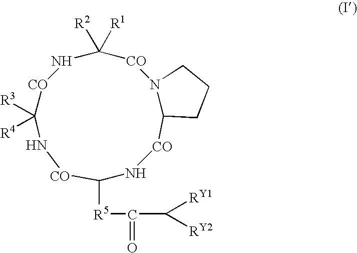
wherein
R¹ is hydrogen,
R² is ar(lower)alkyl optionally substituted with one or more suitable substituent(s),
R³ and R⁴ are each hydrogen or lower alkyl, or
R³ and R⁴ are linked together to form lower alkylene,
R⁵ is lower alkylene or lower alkenylene,
R^{Y1} is optionally protected hydroxy, and
R^{Y2} is lower alkyl,
providing that, when R³ is methyl, R⁴ is methyl or ethyl, R⁵ is pentylene, R^{Y1} is optionally substituted hydroxy and R^{Y2} is methyl, then R² is not unsubstituted benzyl,
or a salt thereof.

14. The cyclic tetrapeptide compound of claim 13, wherein R² is phenyl (lower) alkyl optionally substituted with one or more suitable substituent(s) selected from the group consisting of lower alkoxy, ar(lower)alkyloxy, cyano, hydroxy and halogen,
R³ and R⁴ are each lower alkyl, and
R⁵ is lower alkylene.

15. A pharmaceutical composition containing the cyclic tetrapeptide compound of claim 13 or 14 as an active ingredient, in association with a pharmaceutically acceptable, substantially non-toxic carrier or excipient.

16. The cyclic tetrapeptide compound of claim 13 or 14 for use as a medicament.

17. A histone deacetylase inhibitor comprising a cyclic tetrapeptide compound of the formula (I′): 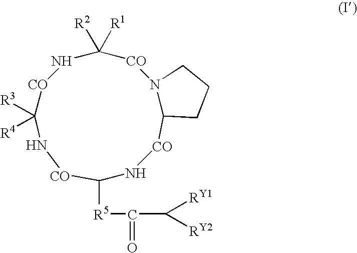
wherein
R¹ is hydrogen,
R² is ar(lower)alkyl optionally substituted with one or more suitable substituent(s),
R³ and R⁴ are each hydrogen or lower alkyl, or
R³ and R⁴ are linked together to form lower alkylene,
R⁵ is lower alkylene or lower alkenylene,
R^{Y1} is optionally protected hydroxy, and
R^{Y2} is lower alkyl,
providing that,
when R³ is methyl, R⁴ is methyl or ethyl, R⁵ is pentylene, R^{Y1} is optionally substituted hydroxy and R^{Y2} is methyl, then R² is not unsubstituted benzyl,
or a salt thereof.

18. Method for inhibiting histone deacetylase, comprising using a cyclic tetrapeptide compound (I′) of claim 17.

19. Use of a cyclic tetrapeptide compound (I′) of claim 17 for the manufacture of a medicament for inhibiting histone deacetylase.

20. A pharmaceutical composition for treating or preventing inflammatory disorders, diabetes, diabetic complications, homozygous thalassemia, fibrosis, cirrhosis, acute promyelocytic leukaemia (APL), organ transplant rejections, autoimmune diseases, protozoal infections or tumors, which comprises, as an active ingredient, a cyclic tetrapeptide compound of the formula (I′): 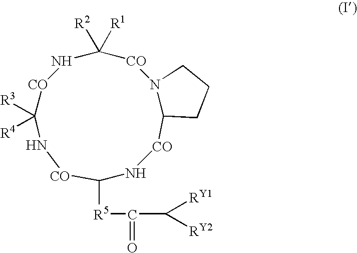
wherein
R¹ is hydrogen,
R² is ar(lower)alkyl optionally substituted with one or more suitable substituent(s),
R³ and R⁴ are each hydrogen or lower alkyl, or
R³ and R⁴ are linked together to form lower alkylene, R⁵ is lower alkylene or lower alkenylene,
R^{Y1} is optionally protected hydroxy, and
R^{Y2} is lower alkyl,
providing that,
when R³ is methyl, R⁴ is methyl or ethyl, R⁵ is pentylene, R^{Y1} is optionally substituted hydroxy and R^{Y2} is methyl, then R² is not unsubstituted benzyl, or a salt thereof.

21. A method for treating or preventing inflammatory disorders, diabetes, diabetic complications, homozygous thalassemia, fibrosis, cirrhosis, acute promyelocytic leukaemia (APL), organ transplant rejections, autoimmune diseases, protozoal infections or tumors, which comprises administering an effective amount of the cyclic tetrapeptide compound (I′) of claim 13 to a human being or an animal.

22. Use of the cyclic tetrapeptide compound (I′) of claim 13 for the manufacture of a medicament for treating or preventing inflammatory disorders, diabetes, diabetic complications, homozygous thalassemia, fibrosis, cirrhosis, acute promyelocytic leukaemia (APL), organ transplant rejections, autoimmune diseases, protozoal infections or tumors.

23. A commercial package comprising the pharmaceutical composition of claim 20 and a written matter associated therewith, the written matter stating that the pharmaceutical composition may or should be used for treating or preventing inflammatory disorders, diabetes, diabetic complications, homozygous thalassemia, fibrosis; cirrhosis, acute promyelocytic leukaemia (APL), organ transplant rejections, autoimmune diseases, protozoal infections or tumors.
